(19) United States
(12) Patent Application Publication
(10) Pub. No.: US 2006/0069132 A1
(43) Pub. Date: Mar. 30, 2006

(54) Azolecarboxamide herbicides

(76) Inventors: Gregory Russell Armel, Bear, DE (US); Mark S. Casini, Newark, DE (US); Dominic Ming-Tak Chan, Wilmington, DE (US); Balreddy Kamireddy, Hockessin, DE (US); Hyeong Baik Kim, Newark, DE (US); Kanu Maganbhai Patel, Wilmington, DE (US); Paula Louise Sharpe, Middletown, DE (US); Thomas Martin Stevenson, Newark, DE (US); Ming Xu, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/096,104

(22) Filed: Mar. 31, 2005

Related U.S. Application Data
(63) Continuation-in-part of application No. PCT/US04/10711, filed on Apr. 7, 2004.

(51) Int. Cl. A61K 31/4196; A61K 31/4192; A61K 31/415; C07D 249/04

(52) U.S. Cl. 514/359; 514/383; 514/400; 514/406; 548/255; 548/269.4; 548/377.1

(57) ABSTRACT

Compounds of Formula I, and their N-oxides and agriculturally suitable salts, are disclosed which are useful for controlling undesired vegetation 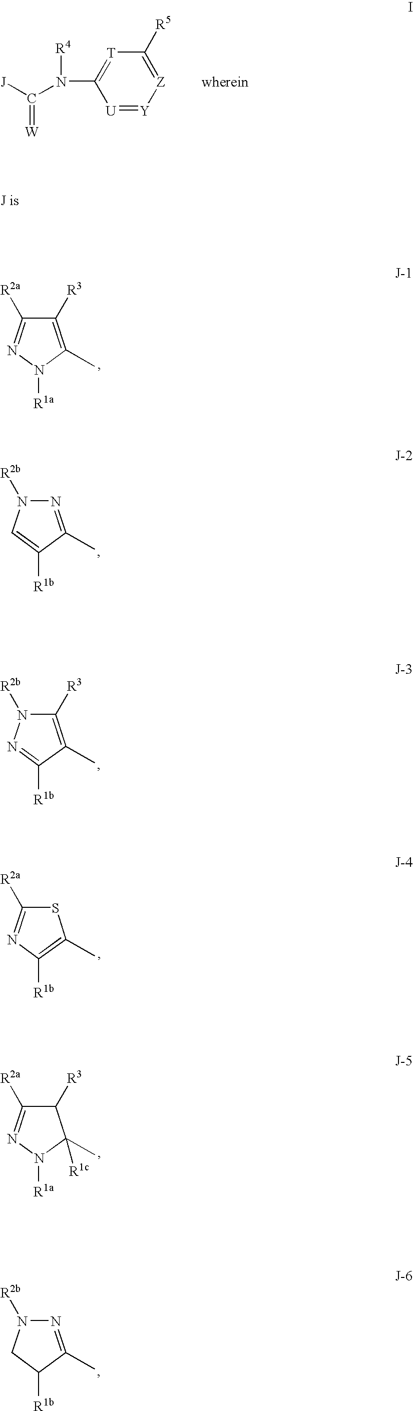 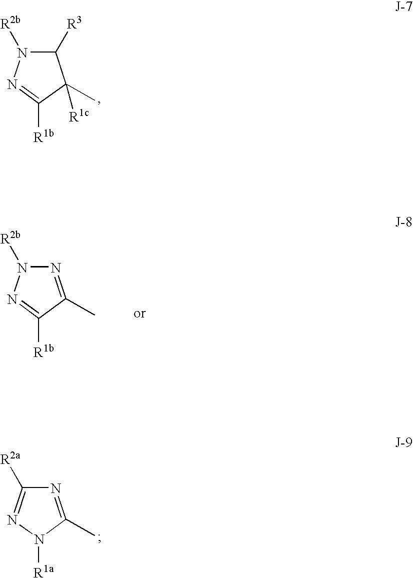
and R^{1a}, R^{1b}, R^{1c}, R^{2a}, R^{2b}, R³, R⁴, R⁵, T, U, W, Y and Z are as defined in the disclosure.
   Also disclosed are compositions containing the compounds of Formula I and a method for controlling undesired vegetation which involves contacting the vegetation or its environment with an effective amount of a compound of Formula I. Also disclosed are mixtures and compositions comprising a herbicidally effective amount of a compound of Formula Iz 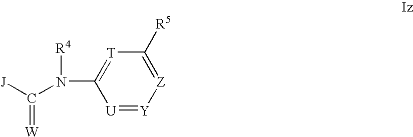
wherein J, R^{1a}, R^{1b}, R^{1c}, R^{2a}, R^{2b}, R³, R⁴, R⁵, T, U, W, Y and Z are as defined in the disclosure; and an effective amount of another herbicide or herbicide safener. Also disclosed is a method for selectively controlling undesired vegetation in a crop that involves contacting the locus of a crop with an effective amount of a compound of Formula Iz and a effective amount of a safener.

### FIELD OF THE INVENTION

This invention relates to certain azolecarboxamides their N-oxides, agriculturally suitable salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, corn (maize), potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds which are more effective, less costly, less toxic, environmentally safer or have different modes of action.

J. J. Parlow, D. A. Mischke and S. S. Woodard, J. Org. Chem. 1997, 62, 5908-5919 and J. J. Parlow, J. Heterocyclic Chem. 1998, 35, 1493-1499 disclose certain pyrazole-carbonylaminobenzene-and pyridinecarboxamides as herbicides. The present Applicants have discovered azolecarboxamides not disclosed by these two publications and which have significantly improved herbicidal utility. Additionally the present Applicants have discovered more efficacious or selective herbicidal compositions and improved methods of weed control from combination of azolecarboxamides with other herbicides and/or herbicide safeners.

### SUMMARY OF THE INVENTION

This invention is directed to a compound of Formula I including all geometric and stereoisomers, N-oxides or agriculturally suitable salts thereof, agricultural compositions containing them and their use as herbicides: 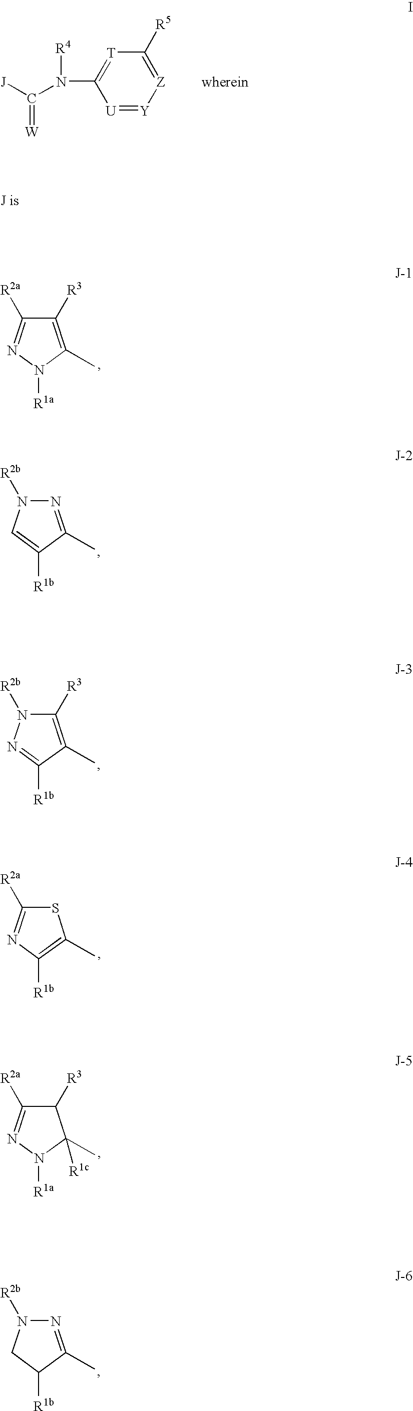 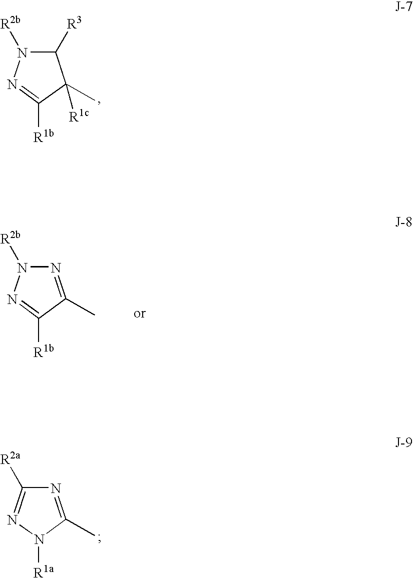
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or
R^{2a} or R^{2b} is taken together with R³ as —C(R^{26a})(R^{26b})—(Y¹)ₛ—(CH₂)ₜ—(Y²)ₙ— or —C(R^{26a})(R^{26b})—(Y¹)ₙ—CH═CH—(Y²)_{w}— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹, C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H, F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C₁-C₄ haloalkyl, C₂-C₄ alkenyl, C₃-C₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1; provided that
(a) when J is J-1, R^{1a} is CH₃ and R⁵ is C(W¹)NR¹⁰R¹¹, C(O)OR¹², COR¹³, OR¹⁶ or S(O)ₘR¹⁷, then at least one of T, U, Y and Z is N or C—F;
(b) when J is J-1, R^{1a} is CH₃, R⁵ is C(W¹)NR¹⁰R¹¹, C(O)OR¹², COR¹³, OR¹⁶ or S(O)ₘR¹⁷ and T is N, then at least one of U, Y and Z is N or C—F;
(c) when J is J-1 and R^{2a} is CF₃, then R⁵ is C(W¹)NR¹⁰R¹¹ or C(NOR¹⁴)R¹⁵, or R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
(d) when J is J-3 and R^{1b} is CF₃, then R⁵ is C(W¹)NR¹⁰R¹¹ or C(NOR¹⁴)R¹⁵, or R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
(e) when J is J-1 or 3-4 and R⁵ is OR¹⁶, then R^{2a} is other than CH₃;
(f) when J is J-3 and R⁵ is OR¹⁶ then R^{2b} is other than CH₃;
(g) when R⁵ is C(W¹)NR¹⁰R¹¹ or C(NOR¹⁴)R¹⁵, then R⁹ is other than alkoxy or alkylthio;
(h) when R⁵ is C(W¹)NR¹⁰R¹¹, then R⁶ is other than alkyl or alkoxy;
(i) when R⁵ is COR¹³, then R^{1a} or R^{1b} is selected from the radicals of the group consisting of C₁-C₃ alkyl, C₁-C₃ fluoroalkyl, C₂-C₃ alkenyl, C₂-C₃ fluoroalkenyl, C₂C₃ alkynyl or C₂-C₃ fluoroalkynyl, each radical unbranched and connected through a terminal end carbon atom to the azole ring;
(j) when R⁵ and R⁶ are taken together as —C(W¹)N(R¹⁰)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(k) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(l) when J is J-2 or J-6, then R⁷ and R⁹ are H;
(m) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(n) when R^{1a} is CH₃ and R⁵ is C(NOR¹⁴)R¹⁵, then R⁷ is other than alkyl;
(O) when T is N, then Z is CR⁹;
(p) when T is N and R⁷ is alkoxy, then R¹¹ is H;
(q) when R⁷ and R⁹ are F, and one of R¹⁰ and R¹¹ is H, then the other of R¹⁰ and R¹¹ is other than H;
(r) when Z is N and one of R¹⁰ and R¹¹ is H, then the other of R¹⁰ and R¹¹ is other than trifluoroethyl;
(s) when J is J-8 and R^{2b} is C₅-C₆ cycloalkyl, then R⁵ is C(O)NR¹⁰R¹¹;
(t) when J is J-8 and R⁷ is other than H, then R^{2b} is tert-butyl and R⁵ is C(O)NR¹⁰R¹¹; and
(u) when J is J-8, then R¹¹ is other than 1-ethylpropyl.

More particularly, this invention pertains to a compound of Formula I, including all geometric and stereoisomers, N-oxides or agriculturally suitable salts thereof. This invention also relates to a herbicidal composition comprising a herbicidally effective amount of a compound of Formula I and at least one of a surfactant, a solid diluent or a liquid diluent. This invention further relates to a method for controlling the growth of undesired vegetation comprising contacting the vegetation or its environment with a herbicidally effective amount of a compound of Formula I (e.g., as a composition described herein). This invention also relates to a method for selectively controlling the growth of undesired vegetation in a crop comprising contacting the locus of the crop with a herbicidally effective amount of a compound of Formula I and an antidotally effective amount of a safener.

The present invention also relates to a herbicidal mixture comprising a herbicidally effective amount of a compound of Formula Iz including all geometric and stereoisomers, N-oxides and agriculturally suitable salts thereof 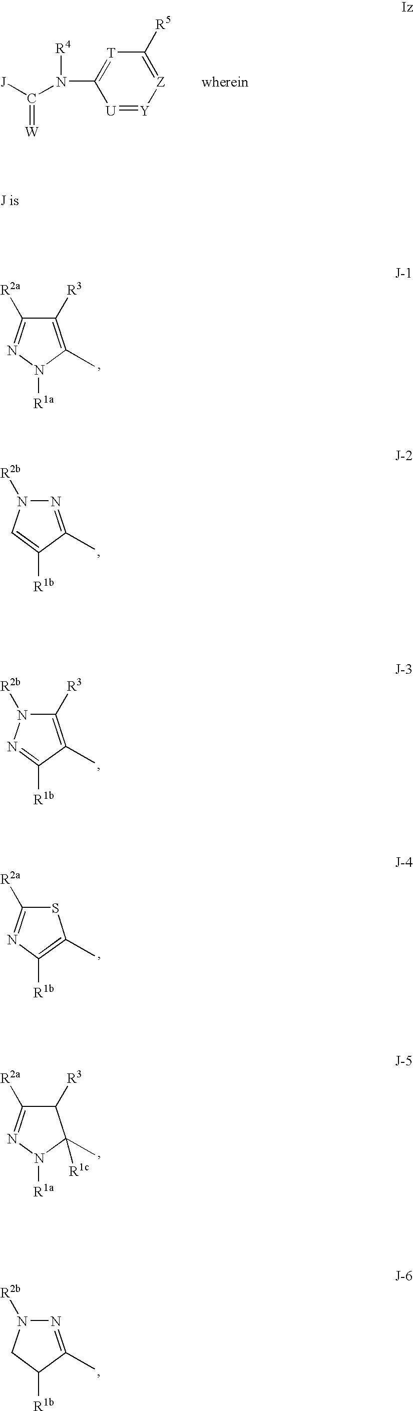 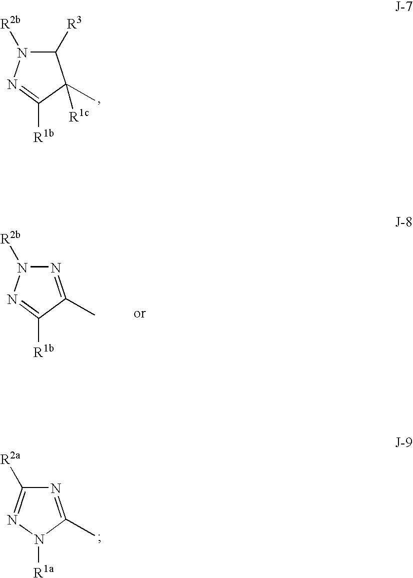
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl; R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or R^{2a} or R^{2b} is taken together with R³ as —C(R^{26a})(R^{26b})—(Y¹)ₛ—(CH₂)ₜ—(Y²)ᵤ— or —C(R^{26a})(R^{26b})—(Y¹)ₙ—CH═CH—(Y²)_{w}— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹, C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H, F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C₁-C₄ haloalkyl, C₂-C₄ alkenyl, C₃-C₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1; provided that
(a) when R⁵ is C(W¹)NR¹⁰R¹¹ or C(NOR¹⁴)R¹⁵, then R⁹ is other than alkoxy or alkylthio;
(b) when R⁵ is C(W¹)NR¹⁰R¹¹, then R⁶ is other than alkyl or alkoxy;
(c) when R⁵ is COR¹³, then R^{1a} or R^{1b} is selected from the radicals of the group consisting of C₁-C₃ alkyl, C₁c₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring;
(d) when R⁵ and R⁶ are taken together as —C(W¹)N(R¹⁰)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(e) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(f) when J is J-2 or J-6, then R⁷ and R⁹ are H;
(g) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(h) when R^{1a} is CH₃ and R⁵ is C(NOR¹⁴)R¹⁵, then R⁷ is other than alkyl;
(i) when T is N, then Z is CR⁹;
(j) when T is N and R⁷ is alkoxy, then R¹¹ is H;
(k) when R⁷ and R⁹ are F, and one of R¹⁰ and R¹¹ is H, then the other of R¹⁰ and R¹¹ is other than H;
(l) when Z is N and one of R¹⁰ and R¹¹ is H, then the other of R¹⁰ and R¹¹ is other than trifluoroethyl;
(m) when J is J-8 and R^{2b} is C₅-C₆ cycloalkyl, then R⁵ is C(O)NR¹⁰R¹¹;
(n) when J is J-8 and R⁷ is other than H, then R^{2b} is tert-butyl and R⁵ is C(O)NR¹⁰R¹¹; and
(o) when J is J-8, then R¹¹ is other than 1-ethylpropyl;
   and an effective amount of at least one additional active ingredient selected from the group consisting of an other herbicide and a herbicide safener. The present invention further relates to a herbicidal composition comprising said herbicidal mixture and at least one of a surfactant, a solid diluent or a liquid diluent.

The present invention also relates to a method for controlling the growth of undesired vegetation comprising contacting the vegetation or its environment with a herbicidally effective amount of a compound of Formula Iz and effective amount of at least one additional active ingredient selected from the group consisting of an other herbicide and a herbicide safener (e.g., in the form of the aforedescribed herbicidal mixture or herbicidal composition). A particular aspect of the present invention relates to a method for selectively controlling the growth of undesired vegetation in a crop comprising contacting the locus of a crop with an effective amount of a compound of Formula Iz and an antidotally effective amount of a herbicide safener (e.g., safener applied as a seed treatment).

### DETAILS OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components of the invention. This is done merely for convenience and to give a general sense of the invention. This description should be read to include one or at least one and the singular also includes the plural unless it is obvious that it is meant otherwise.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. The term “1-2 alkyl” indicates that one or two of the available positions for that substituent may be alkyl which are independently selected. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy and pentoxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkylthio” includes branched or straight-chain alkylthio moieties such as methylthio, ethylthio, and the different propylthio, butylthio and pentylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl isomers. “Alkenylthio”, “alkenylsulfinyl”, “alkenylsulfonyl”, “alkynylthio”, “alkynylsulfinyl”, “alkynylsulfonyl”, and the like, are defined analogously to the above examples. “Cycloalkyl” includes, for example, cyclopropyl, cyclobutyl, cyclopentyl, and cyclohexyl. The term “cycloalkoxy” includes the same groups linked through an oxygen atom such as cyclopropyloxy and cyclobutyloxy. Examples of “cycloalkylalkyl” include cyclopropylmethyl, cyclopentylethyl, and other cycloalkyl moieties bonded to straight-chain or branched alkyl groups. “Cycloalkylalkoxy” includes cyclopropylmethoxy. “Alkylcycloalkyl” denotes alkyl substitution on a cycloalkyl moiety. Examples include 4-methylcyclohexyl and 3-ethylcyclopentyl. The term “carbocyclic ring” denotes a ring wherein the atoms forming the ring backbone and selected only from carbon. “Saturated carbocyclic” refers to a ring having a backbone consisting of carbon atoms linked to one another by single bonds; unless otherwise specified, the remaining carbon valences are occupied by hydrogen atoms. The term “aromatic ring system” denotes fully unsaturated carbocycles and heterocycles in which the polycyclic ring system is aromatic. Aromatic indicates that each of ring atoms is essentially in the same plane and has a p-orbital perpendicular to the ring plane, and in which (4n+2)π electrons, where n is 0 or a positive integer, are associated with the ring to comply with Huckel's rule. The term “aromatic carbocyclic ring system” includes fully aromatic carbocycles and carbocycles in which at least one ring of a polycyclic ring system is aromatic. The term “nonaromatic carbocyclic ring system” denotes fully saturated carbocycles as well as partially or fully unsaturated carbocycles wherein none of the rings in the ring system are aromatic. The terms “aromatic heterocyclic ring system” and “heteroaromatic ring” include fully aromatic heterocycles and heterocycles in which at least one ring of a polycyclic ring system is aromatic. The term “nonaromatic heterocyclic ring system” denotes fully saturated heterocycles as well as partially or fully unsaturated heterocycles wherein none of the rings in the ring system are aromatic. The heterocyclic ring systems can be attached through any available carbon or nitrogen by replacement of a hydrogen on said carbon or nitrogen. One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. Synthetic methods for the preparation of N-oxides of heterocycles are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethydioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

The term “halogen”, either alone or in compound words such as “haloalkyl”, includes fluorine, chlorine, bromine or iodine. The term “1-2 halogen” indicates that one or two of the available positions for that substituent may be halogen which are independently selected. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂. Similarly, “fluoroalkyl”, “fluoroalkenyl” and “fluoroalkynyl” may be partially or fully substituted with fluorine atoms.

The total number of carbon atoms in a substituent group is indicated by the “Cᵢ-Cⱼ” prefix where i and j are numbers from 1 to 6. For example, C₁-C₃ alkyl designates methyl through propyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. Examples of “alkylcarbonyl” include C(O)CH₃, C(O)CH₂CH₂CH₃ and C(O)CH(CH₃)₂. Examples of “alkoxycarbonyl” include CH₃C(═O), CH₃CH₂C(═O), CH₃CH₂CH₂C(═O), (CH₃)₂CHOC(═O) and the different butoxy- or pentoxycarbonyl isomers.

When a compound is substituted with a substituent bearing a subscript that indicates the number of said substituents can exceed 1, said substituents (when they exceed 1) are independently selected from the group of defined substituents. Further, when the subscript indicates a range, e.g. (R)ᵢ₋ⱼ, then the number of substituents may be selected from the integers between i and j inclusive.

When a group contains a substituent which can be hydrogen, for example R⁶ or R¹⁰, then, when this substituent is taken as hydrogen, it is recognized that this is equivalent to said group being unsubstituted. When a position on a group is said to be “not substituted” or “unsubstituted”, then hydrogen atoms are attached to take up any free valency.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the present invention comprises compounds selected from Formula I, N-oxides and agriculturally suitable salts thereof. The compounds of the invention may be present as a mixture of stereoisomers, individual stereoisomers, or as an optically active form.

The agriculturally suitable salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. The agriculturally suitable salts of the compounds of the invention also include those formed with strong bases (e.g., hydrides or hydroxides of sodium, potassium or lithium). One skilled in the art recognizes that because in the environment and under physiological conditions salts of the compounds of the invention are in equilibrium with their corresponding nonsalt forms, agriculturally suitable salts share the biological utility of the nonsalt forms.

Embodiments of the present invention include:
Embodiment 1. A compound of Formula I wherein when J is J-1 and R^{1a} is CH₃ then at least one of T and U is N or C—F.
Embodiment 2. A compound of Embodiment 1 wherein when J is J-1 and R^{1a} is CH₃ then at least one of T and U is C—F.
Embodiment 3. A compound of Formula I wherein when J is J-1, R^{1a} is CH₃ and T is N then U is N or C—F.
Embodiment 4. A compound of Embodiment 3 wherein when J is J-1, R^{1a} is CH₃ and T is N then U is C—F.
Embodiment 5. A compound of Formula I wherein at most one of T, U, Y and Z is N.
Embodiment 6. A compound of Formula I wherein W is O.
Embodiment 7. A compound of Formula I wherein J is J-1, J-2, J-3, J-4, J-5, J-8 or J-9.
Embodiment 8. A compound of Formula I wherein J is J-1, J-2, J-3, J-4, J-5 or J-8.
Embodiment 9. A compound of Formula I wherein J is J-1, J-3, J-5, J-8 or J-9.
Embodiment 10. A compound of Formula I wherein J is J-1, J-3, J-5 or J-8.
Embodiment 11. A compound of Formula I wherein J is J-1, J-3, J-8 or J-9.
Embodiment 12. A compound of Formula I wherein J is J-1, J-3 or J-5.
Embodiment 13. A compound of Formula I wherein J is J-1 or J-3.
Embodiment 14. A compound of Formula I wherein R^{1a} is C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl.
Embodiment 15. A compound of Formula I wherein R^{1a} or R^{1b} is selected from the radicals in the group consisting of C₁-C₃ alkyl, C₁-C₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring.
Embodiment 16. A compound of Embodiment 15 wherein R^{1a} is CH₃, CH₂CH₃, CH₂CH₂F, CH₂CHF₂, CH₂CF₃ or CH═CH₂.
Embodiment 17. A compound of Embodiment 16 wherein R^{1a} is CH₂CH₃, CH₂CH₂F, CH₂CHF₂, CH₂CF₃ or CH═CH₂.
Embodiment 18. A compound of Embodiment 17 wherein R^{1a} is CH₂CH₃ or CH₂CF₃.
Embodiment 19. A compound of Embodiment 15 wherein R^{1b} is CH₃, CH₂CH₃, CH₂CH₂F, CH₂CHF₂, CH₂CF₃ or CH═CH₂.
Embodiment 20. A compound of Embodiment 19 wherein R^{1b} is CH₂CH₃, CH₂CH₂F, CH₂CHF₂, CH₂CF₃ or CH═CH₂.
Embodiment 21. A compound of Embodiment 20 wherein R^{1b} is CH₂CH₃ or CH₂CF₃.
Embodiment 22. A compound of Embodiment 21 wherein R^{1b} is CH₃, CH₂CH₃ or CH₂CF₃.
Embodiment 23. A compound of Formula I wherein R^{2a} or R^{2b} is tert-butyl, isopropyl or cyclopropyl.
Embodiment 24. A compound of Embodiment 23 wherein R^{2a} or R^{2b} is tert-butyl or isopropyl.
Embodiment 25. A compound of Formula I wherein Y¹ and Y² are independently CH₂ or O;
Embodiment 26. A compound of Embodiment 25 wherein the sum of s, t and u is 2 and the sum of v and w is 0;
Embodiment 27. A compound of Embodiment 25 wherein R^{26a} is C₁-C₂ alkyl.
Embodiment 28. A compound of Embodiment 27 wherein R^{26a} and R^{26b} are CH₃.
Embodiment 29. A compound of Formula I wherein R³ is H.
Embodiment 30. A compound of Formula I wherein R⁴ is H.
Embodiment 31. A compound of Formula I wherein the carbon atom of R¹² linking to oxygen is bonded to at least one hydrogen atom.
Embodiment 32. A compound of Formula I wherein R⁵ is CONR¹⁰R¹¹ or C(O)OR¹²;
R¹⁰ is H or C₁-C₄ alkyl; R¹¹ is C₁-C₄ alkyl; or R¹⁰ and R¹¹ are taken together as —CH₂CH═CH₂CH—; and R¹² is C₁-C₃ alkyl.
Embodiment 33. A compound of Embodiment 32 wherein R⁵ is CONR¹⁰R¹¹; R¹⁰ is H or C₁-C₂ alkyl; and R¹¹ is C₁-C₃ alkyl; or R¹⁰ and R¹¹ are taken together as —CH₂CH═CH₂CH—.
Embodiment 34. A compound of Formula I wherein R⁵ is CONR¹⁰R¹¹ or C(O)OR¹².
Embodiment 35. A compound of Formula I wherein R⁵ is CONR¹⁰R¹¹.
Embodiment 36. A compound of Formula I wherein R¹⁰ is C₁-C₄ alkyl and R^{1l} is H, C₁-C₄ alkyl, C₁-C₄ alkylcarbonyl or C₁-C₄ alkoxycarbonyl; or R¹⁰ and R¹¹ are taken together as —CH₂CH═CH₂CH—.
Embodiment 37. A compound of Formula I wherein R¹⁰ is C₁-C₃ alkyl and R¹¹ is H, C₁-C₂ alkyl, C₁-C₃ alkylcarbonyl or C₁-C₃ alkoxycarbonyl; or R¹⁰ and R¹¹ are taken together as —CH₂CH═CH₂CH—.
Embodiment 38. A compound of Formula I wherein R¹⁰ is C₁-C₄ alkyl and R^{1l} is H or C₁-C₄ alkyl; or R¹⁰ and R¹¹ are taken together as —CH₂CH═CH₂CH—.
Embodiment 39. A compound of Formula I wherein R¹⁰ is C₁-C₃ alkyl and R¹¹ is H or C₁-C₂ alkyl; or R¹⁰ and R¹¹ are taken together as —CH₂CH═CH₂CH—.
Embodiment 40. A compound of Formula I wherein R⁵ is C(O)OR¹².
Embodiment 41. A compound of Formula I wherein R¹² is C₁-C₃ alkyl.
Embodiment 42. A compound of Formula I wherein R⁶ is H or F.
Embodiment 43. A compound of Formula I wherein R⁷ is H or F.
Embodiment 44. A compound of Formula I wherein R⁸ and R⁹ are H or F.
Embodiment 45. A compound of Formula I wherein T is C—F or N.
Embodiment 46. A compound of Formula I wherein U is C—F or N.
Embodiment 47. A compound of Formula I wherein J is J-1.
Embodiment 48. A compound of Formula I wherein J is J-2.
Embodiment 49. A compound of Formula I wherein J is J-3.
Embodiment 50. A compound of Formula I wherein J is J-4.
Embodiment 51. A compound of Formula I wherein J is J-5.
Embodiment 52. A compound of Formula I wherein J is J-6.
Embodiment 53. A compound of Formula I wherein J is J-7.
Embodiment 54. A compound of Formula I wherein J is J-8.
Embodiment 55. A compound of Formula I wherein J is J-9.
Embodiment 56. A compound of Formula I wherein when J is J-1, then R^{1a} is other than CH₃.
Embodiment 57. A compound of Formula I wherein when J is J-1, then R^{1a} is CH₂CH₃, CH₂CH₂F, CH₂CHF₂, CH₂CF₃ or CH═CH₂.
Embodiment 58. A compound of Formula I wherein when J is J-1, then R^{1a} is CH₂CH₃ or CH₂CF₃.
Embodiment 59. A compound of Formula I wherein when J is J-5, then R^{1a} is CH₂CH₃, CH₂CH₂F, CH₂CHF₂, CH₂CF₃ or CH═CH₂.
Embodiment 60. A compound of Formula I wherein when J is J-5, then R^{1a} is CH₂CH₃ or CH₂CF₃.
Embodiment 61. A compound of Formula I wherein when J is J-1, then R^{2a} is other than CF₃.
Embodiment 62. A compound of Formula I wherein when J is J-1, then R^{2a} is other than haloalkyl.
Embodiment 63. A compound of Formula I wherein when J is J-3, then R^{1b} is other than CF₃.
Embodiment 64. A compound of Formula I wherein when J is J-3, then R^{1b} is other than fluoroalkyl.
Embodiment 65. A compound of Formula I wherein when J is J-1 and R⁵ is OR¹⁶, then R^{2a} is other than C₁-C₂ alkyl;
Embodiment 66. A compound of Formula I wherein when J is J-1, then R⁵ is other than OR¹⁶.
Embodiment 67. A compound of Formula I wherein when J is J-3 and R⁵ is OR¹⁶, then R^{2b} is other than C₁-C₂ alkyl;
Embodiment 68. A compound of Formula I wherein when J is J-3, then R⁵ is other than OR¹⁶.
Embodiment 69. A compound of Formula I wherein when J is J-4 and R⁵ is OR¹⁶, then R^{2a} is other than C₁-C₂ alkyl;
Embodiment 70. A compound of Formula I wherein when J is J-4, then R⁵ is other than OR¹⁶.
Embodiment 71. A compound of Formula I wherein when J is J-4 and R⁵ is OR¹⁶, then R^{1b} is other than 1-fluoroethyl.
Embodiment 72. A compound of Formula I wherein when J is J-4, then R^{1b} is other than 1-fluoroethyl.
Embodiment 73. A compound of Formula I wherein when J is J-4 and R⁵ is OR¹⁶, then R^{1b} is CH₂CH₃, CH₂CH₂F, CH₂CHF₂, CH₂CF₃ or CH═CH₂.
Embodiment 74. A compound of Formula I wherein when J is J-4 and R⁵ is OR¹⁶, then R^{1b} is CH₂CH₃ or CH₂CF₃.
Embodiment 75. A compound of Formula I wherein R⁵ is other than OR¹⁶.
Embodiment 76. A compound of Formula I wherein R⁹ is other than fluoroalkyl.
Embodiment 77. A compound of Formula I wherein when J is J-3, then R⁵ is other than —CN.
Embodiment 78. A compound of Formula I wherein when J is J-4, then R⁵ is other than —CN.
Embodiment 79. A compound of Formula I wherein R⁵ is other than —CN.
Embodiment 80. A compound of Formula I wherein when J is J-3 and R⁵ is —CN, then R⁹ is other than alkoxy.
Embodiment 81. A compound of Formula I wherein when J is J-3 then R⁹ is other than alkoxy.
Embodiment 82. A compound of Formula I wherein R⁹ is other than alkoxy.
Embodiment 83. A compound of Formula I wherein R⁹ is H.
Embodiment 84. A compound of Formula I wherein Z is CR⁹.
Embodiment 85. A compound of Formula I wherein R¹¹ is other than 1-ethylpropyl.
Embodiment 86. A compound of Formula I wherein when J is J-9, R⁵ is C(W¹)NR¹⁰R¹¹, C(O)OR¹² or OR¹⁶.
Embodiment 87. A compound of Formula I wherein when J is J-1, then R^{2a} is other than haloalkenyl.
Embodiment 88. A compound of Formula I wherein when J is J-1, then R^{2a} is other than haloalkynyl.
Embodiment 89. A compound of Formula I wherein when J is J-1, then R^{2a} is other than halocycloalkyl.
Embodiment 90. A compound of Formula I wherein when J is J-1, then R^{2a} is tert-butyl, isopropyl, cyclopropyl or 1-methylcyclopropyl
Embodiment 91. A compound of Formula I wherein R^{2a} or R^{2b} is tert-butyl, isopropyl, cyclopropyl or 1-methylcyclopropyl.

Of note is a compound of Formula I wherein J is J-1 and R^{1a} is H, which is particularly useful as a synthetic intermediate.

Combinations of Embodiments 1-91 are illustrated by:
Embodiment A. A compound of Formula I wherein J is J-1, J-2, J-3, J-4, J-5, J-8 or J-9.
Embodiment B. A compound of Embodiment A wherein R^{1a} is CH₂CH₃, CH₂CH₂F, CH₂CHF₂, CH₂CF₃ or CH═CH₂; R^{1b} is CH₃, CH₂CH₃, CH₂CH₂F, CH₂CHF₂, CH₂CF₃ or CH═CH₂; R^{2a} or R^{2b} is tert-butyl, isopropyl or cyclopropyl; R³ is H; R⁴ is H; and W is O.
Embodiment C. A compound of Embodiment B wherein at most one of T, U, Y and Z is N.
Embodiment D. A compound of Embodiment C wherein R⁵ is CONR¹⁰R¹¹ or C(O)OR¹²; R¹⁰ is H or C₁-C₄ alkyl; R¹¹ is C₁-C₄ alkyl; or R¹⁰ and R¹¹ are taken together as —CH₂CH═CH₂CH—; and R¹² is C₁-C₃ alkyl.
Embodiment E. A compound of Embodiment D wherein R⁶ is H or F and R⁷ is H or F.
Embodiment F. A compound of Embodiment E wherein J is J-1, J-3, J-5 or J-8.
Embodiment G. A compound of Embodiment F wherein R⁵ is CONR¹⁰R¹¹; R¹⁰ is H or C₁-C₂ alkyl; and R¹¹ is C₁-C₃ alkyl; or R¹⁰ and R¹¹ are taken together as —CH₂CH═CH₂CH—.
Embodiment H. A compound of Embodiment G wherein R^{2a} is tert-butyl or isopropyl; and R⁸ and R⁹ are H or F.
Embodiment I. A compound of Formula I wherein at most one of T, U, Y and Z is N; R^{1a} or R^{1b} is selected from the radicals in the group consisting of C₁-C₃ alkyl, C₁-C₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring; R⁶ is H or F; R⁷ is H or F; R⁴ is H; R^{26a} is C₁-C₂ alkyl; W is O; Y¹ and Y² are independently CH₂ or O; the sum of s, t and u is 2; and the sum of v and w is 0.
Embodiment J. A compound of Embodiment I wherein R^{1a} is CH₂CH₃, CH₂CH₂F, CH₂CHF₂, CH₂CF₃ or CH═CH₂; R^{1b} is CH₃, CH₂CH₃, CH₂CH₂F, CH₂CHF₂, CH₂CF₃ or CH═CH₂; R^{2a} or R^{2b} is tert-butyl, isopropyl or cyclopropyl; and R³ is H.
Embodiment K. A compound of Embodiment J wherein R⁵ is CONR¹⁰R¹¹ or C(O)OR¹²; R¹⁰ is H or C₁-C₄ alkyl; R¹¹ is C₁-C₄ alkyl; or R¹⁰ and R¹¹ are taken together as —CH₂CH═CH₂CH—; and R¹² is C₁-C₃ alkyl.
Embodiment L. A compound of Embodiment K wherein R^{2a} or R^{2b} is tert-butyl or isopropyl.
Embodiment M. A compound of Embodiment L wherein J is J-1, J-2, J-3, J-4, J-5, J-8 or J-9.
Embodiment N. A compound of Embodiment M wherein R⁵ is CONR¹⁰R¹¹; R¹⁰ is H or C₁-C₂ alkyl; and R¹¹ is C₁-C₃ alkyl; or R¹⁰ and R¹¹ are taken together as —CH₂CH═CH₂CH—.
Embodiment O. A compound of Embodiment N wherein R⁸ and R⁹ are H or F.
Embodiment P. A compound of Embodiment 0 wherein J is J-1, J-3, J-5 or J-8.

Specific embodiments include the following compounds of Embodiment I:
3-(1,1-dimethylethyl)-1-ethyl-N-[5-[(ethylamino)carbonyl)-2-fluorophenyl]-1H-pyrazole-5-carboxamide;
N-[5-[(dimethylamino)carbonyl]-2-fluorophenyl]-3-(1,1-dimethylethyl)-1-ethyl-1H-pyrazole-5-carboxamide;
2-[[(3-(1,1-dimethylethyl)-1-ethyl-1H-pyrazol-5-yl]carbonyl]amino]-N,N-dimethyl-4-pyridinecarboxamide;
2-[[[3-(1,1-dimethylethyl)-1-ethyl-1H-pyrazol-5-yl]carbonyl]amino]-N-ethyl-4-pyridinecarboxamide;
N-[5-[(dimethylamino)carbonyl]-2-fluorophenyl]-1-ethyl-3-(1-methylethyl)-1H-pyrazole-5-carboxamide;
N-[5-[(dimethylamino)carbonyl]-2-fluorophenyl]-1-(1,1-dimethylethyl)-3-ethyl-1H-pyrazole-4-carboxamide;
3-(1, 1-dimethylethyl)-1-(2-fluoroethyl)-N-[3-[(1E)-1-(hydroxyimino)ethyl]phenyl]-1H-pyrazole-5-carboxamide;
3-(1,1-dimethylethyl)-1-ethyl-N-[5-[(ethylmethylamino)carbonyl]-2-fluorophenyl]-1H-pyrazole-5-carboxamide;
3-(1,1-dimethylethyl)-1-ethyl-N-[3-[(ethylamino)carbonyl]-4-fluorophenyl]-1H-pyrazole-5-carboxamide;
N-[5-[(2,5-dihydro-1H-pyrrol-1-yl)carbonyl]-2-fluorophenyl]-3-(1,1-dimethylethyl)-1-ethyl-1H-pyrazole-5-carboxamide;
3-(1,1-dimethylethyl)-1-ethyl-N-[3-(trifluoromethoxy)phenyl]-1H-pyrazole-5-carboxamide;
N-[3-[(dimethylamino)carbonyl]phenyl]-2-(1,1-dimethylethyl)-5-methyl-2H-1,2,3-triazole-4-carboxamide; and
N-[3-[(dimethylamino)carbonyl]phenyl]-2-(1,1-dimethylethyl)-5-ethyl-2H-1,2,3-triazole-4-carboxamide.

Of note are compounds of Formulae I or Iz wherein J is J-1, J-2, J-3 or J-4 wherein R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkyl-alkyl. Also of note are compounds of Formula I or Iz wherein J is J-1, J-2, J-3, J-4, J-5, J-6 or J-7. Also of note are compounds of Formula I or Iz wherein J is J-1, J-2, J-3, J-4, J-5, J-6, J-7 or J-8. Also of note are compounds of Formula I or Iz wherein R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵; R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl; R³ is H, F or C₁-C₂ alkyl; and R⁵ is C(W¹)NR¹⁰R¹¹, C(O)OR¹², COR¹³, C(NOR¹⁴)R¹⁵, —CN, OR¹⁶, S(O)ₘR¹⁷ or S(O)₂NR¹⁸R¹⁹. Also of note are compounds of Formula I or Iz wherein R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl.

Also noteworthy as embodiments are herbicidal compositions of the present invention comprising the compounds of embodiments described above.

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above.

This invention also relates to a method for selectively controlling the growth of undesired vegetation in a crop comprising contacting the locus of the crop with a herbicidally effective amount of the compounds of the invention and an antidotally effective amount of a safener. Of note as embodiments relating to methods of use are those involving the compounds of embodiments described above.

The compounds of Formulae I and Iz can be prepared by one or more of the following methods and variations as described in Schemes 1 through 22 and accompanying text. Formula I is a subgenus of Formula Iz; Formulae I and Iz share the same substituent group definitions, but the scope of Formula Iz is not constrained by provisos (a) and (b) of Formula I. The definitions of J, W, R^{1a}, R^{1b}, R^{1c}, R^{2a}, R^{2b}, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R^{26a}, R^{26b}, R²⁷, R^{28a} R^{28b}, W, W¹, T, U, Y, Z, m, n, s and v in the compounds of Formulae I through Ig, Iz and 1 through 63 below are as defined above in the Summary of the Invention unless otherwise indicated. Compounds of Formulae Ia through Ig are various subsets of the compounds of Formulae I and Iz, compounds of Formula 2a and 2b are subsets of the compounds of Formula 2, and compounds of Formulae 17a through 17l are subsets of the compounds of Formula 17.

Compounds of Formula Ia (Formula I or Iz wherein W is O) can be prepared by coupling the appropriately substituted azole acyl chloride of Formula 1 with the appropriately substituted amino compound of Formula 2 as shown in Scheme 1. 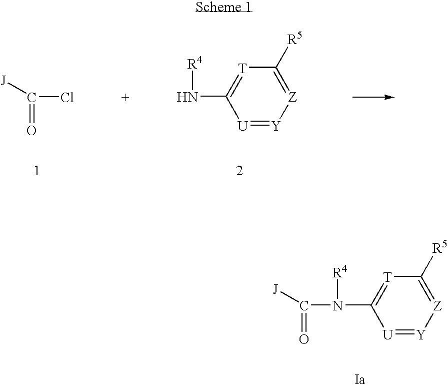
The reaction is carried out in an anhydrous aprotic solvent such as dichloromethane or tetrahydrofuran, preferably in the presence of a base such as triethylamine, pyridine, 4-(dimethylamino)pyridine or N,N-diisopropylethylamine, at a temperature typically between room temperature and 70° C. to provide the amide of Formula Ia. When R⁴ is alkylcarbonyl or alkoxycarbonyl, a strong base such as sodium hydroxide and phase transfer conditions such as those described by M. J. Haddadin et al., Heterocycles 1984, 22, 773 may be advantageous. The reaction of Scheme 1 is illustrated in Step F of Example 1, Step C of Example 4, Step D of Example 7, Step D of Example 8, Step C of Example 12, Step B of Example 13, Step D of Example 14, Step C of Example 15, Step C of Example 16, Step D of Example 19, and Step E of Example 25, which follow.

Alternatively, compounds of Formula Ia can be prepared by coupling the appropriately substituted azole carboxylic acid of Formula 3 with appropriately substituted amino compound of Formula 2 as shown in Scheme 2. 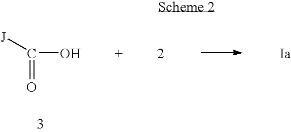
This reaction is carried out in the presence of a dehydrating coupling reagent such as dicyclohexyl carbodiimide, 1-(3-dimethylaminopropyl)-3-ethylcarbodiimide, 1-propane-phosphonic acid cyclic anhydride or carbonyl diimidazole in the presence of a base such as triethylamine, pyridine, 4-(dimethylamino)pyridine or N,N-diisopropylethylamine in an anhydrous aprotic solvent such as dichloromethane or tetrahydrofuran at a temperature typically between room temperature and 70° C. The method of Scheme 2 is illustrated in Step D of Example 10, Step C of Example 17, Example 18, Step B of Example 20 and Step E of Example 22.

As a further method, an ester of a carboxylic acid of Formula 3 (identified as Formula 17 below) can be condensed with a substituted amino compound of Formula 2 to provide the compound of Formula Ia by heating in a high-boiling inert solvent such as α,α,α-trifluorotoluene. This method is illustrated in Step C of Example 30.

As shown in Scheme 3, compounds of Formula Ib (Formula I or Iz wherein W is S) can be prepared from corresponding compounds of Formula Ia by treatment with a thionating reagent such as P₂S₅ (see for example, E. Klingsberg et al., J. Am. Chem. Soc. 1951, 72, 4988; E. C. Taylor Jr. et al., J. Am. Chem. Soc. 1953, 75, 1904; R. Crossley et al., J. Chem. Soc. Perkin Trans. 1 1976, 977; J. Voss et al., Justus Liebigs Ann. Chem. 1968, 716, 209) or Lawesson's Reagent (2,5-bis(4-methoxyphenyl)-1,3-dithia-2,4-diphosphetane-2,4-disulfide; see, for example, S. Prabhakar et al. Synthesis, 1984 (10), 829).

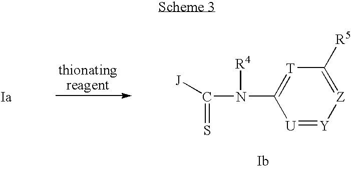

Alternatively, compounds of Formula Ib can be directly prepared from the corresponding carboxylic acid of Formula 3 and amino compound of Formula 2 by treatment with (EtO)₂P(S)SH according to the general procedure of N. Borthakur et al., Tetrahedron Lett. 1995, 36(37), 6745. Also, compounds of Formula Ia or Ib wherein R⁴ is alkyl, alkylcarbonyl, alkoxycarbonyl, alkoxyalkyl or alkylthioalkyl can be prepared from the corresponding compounds of Formula Ia or Ib wherein R⁴ is H by treatment with the appropriate alkylating or acylating reagents in the presence of base using methods well known in the art.

Acyl chlorides of Formula 1 can be prepared from the carboxylic acids of Formula 3 by using methods well known in the art such as treatment with oxalyl chloride and catalytic N,N-dimethylformamide in dichloromethane or treatment with thionyl chloride. This preparation is illustrated in Step E of Example 1, Step C of Example 12, Step B of Example 13, Step D of Example 14, and Step E of Example 25.

In some instances compounds of Formula I (or Iz) can be prepared from other compounds of Formula I (or Iz). For example, a compound of Formula Ic wherein R³⁰ is NR¹⁰R¹¹ or OR¹² (Formula I or Iz wherein R⁵ is C(O)NR¹⁰R¹¹ or C(O)OR¹²) can be prepared from the corresponding carboxylic acid of Formula 4, which is in turn prepared from a compound of Formula Ic wherein R³⁰ is OR¹² as shown in Scheme 4. 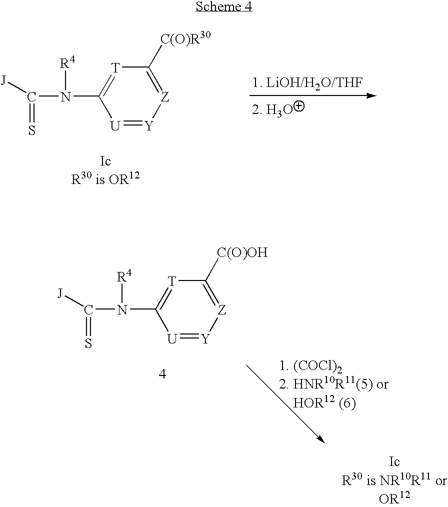
In this method, the ester compound of Formula Ic wherein R³⁰ is OR¹² is converted to the corresponding carboxylic acid of Formula 4 by general procedures well known in the art such as by treatment with aqueous lithium hydroxide in tetrahydrofuran, followed by acidification. The carboxylic acid of Formula 4 is then converted to the corresponding carboxamide of Formula Ic wherein R³⁰ is NR¹⁰R¹² or ester of Formula Ic wherein R³⁰ is OR¹² by amidation or esterification procedures well known in the art. One procedure illustrated in Scheme 4 involves conversion of the carboxylic acid of Formula 4 to an intermediate carbonyl chloride by treatment with oxalyl chloride preferably in the presence of N,N-dimethylformamide and an inert solvent such as dichloromethane, and then contacting the intermediate carbonyl chloride with the appropriate amine of Formula 5 or alcohol of Formula 6 to prepare the carboxamide or ester, respectively. As an alternative to preparing the intermediate carbonyl chloride, a dehydrating coupling reagent can be used analogous to the method of Scheme 2. The method of Scheme 4 is illustrated in Examples 2, 3, 5, 6 and 9, Steps A and B of Example 11, and Example 23.

In other instances compounds of Formula I (or Iz) can be prepared from compounds structurally related to Formula I (or Iz). For example, as shown in Scheme 5, compounds of Formula Id can be prepared from corresponding compounds of Formula 7 by treatment with the corresponding sulfonating reagent of Formula 8 wherein X¹ is a leaving group such as halogen or OS(O)₂R²⁷. For reason of cost, X¹ is preferably Cl. 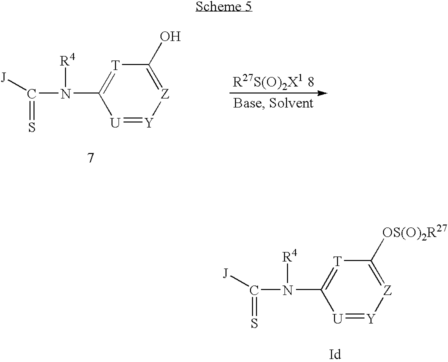
The reaction is conducted in the presence of a base such as pyridine, triethylamine or 4-(dimethylamino)pyridine in solvents such as dichloromethane or tetrahydrofuran at temperatures typically between 0 and 70° C. under an inert atmosphere. Compounds of Formula 7 can be prepared by methods analogous to Schemes 1 and 2, starting with the appropriate amino compound analogous to Formula 2 wherein R⁵ is replaced by a hydroxy group. Although the hydroxy group can be converted to a protecting group before the reaction with the compound of Formulae 1 or 3 and then deprotected to give the compound of Formula 7, such protection is generally unnecessary, because the amino group is more reactive than the hydroxy group.

As shown in Scheme 6, compounds of Formula Ie can be prepared from corresponding compounds of Formula 7 by treatment with the corresponding phosphorating reagent of Formula 9 wherein X² is a leaving group such as halogen. For reason of cost, X² is preferably Cl. 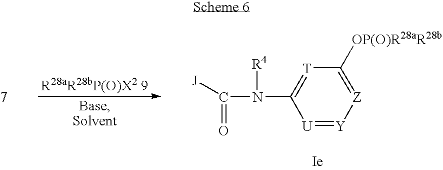
The reaction is conducted in the presence of a base such as pyridine, triethylamine or 4-(dimethylamino)pyridine in solvents such as dichloromethane or tetrahydrofuran at temperatures typically between 0 and 70° C. under an inert atmosphere.

Compounds of Formula I (or Iz) can also be prepared from other compounds of Formula I (or Iz) wherein substituents on the J groups are introduced or elaborated. For example, halogens can be attached using electrophilic addition reactions. Example 21 illustrates the addition of fluorine as R³ wherein J of Formula I (or Iz) is J-1.

Carboxylic acids of Formula 3 can be prepared from corresponding esters of Formula: 17 wherein R³¹ is a carbon-based radical such as alkyl (e.g., methyl, ethyl), benzyl, etc. as shown in Scheme 7. 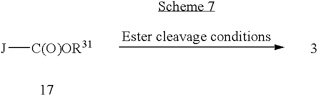
A wide range of ester cleavage conditions known in the art can be used for this method. Particularly suitable are conditions involving treatment with hydroxide, such as aqueous sodium hydroxide or aqueous lithium hydroxide in tetrahydrofuran, followed by acidification, typically with a strong mineral acid such as hydrochloric or sulfuric acid. For cleaving esters of Formula 17 wherein R³¹ is benzyl, hydrogenation over palladium catalyst according to general procedures known in the art can be particularly advantageous. The method of Scheme 7 is illustrated in Step D of Example 1, Step B of Example 12, Step A of Example 13, Step C of Example 14, and Step D of Example 22, and Step D of Example 25.

Carboxylic esters of Formula 17a (Formula 17 wherein J is J-1 and R³¹ is ethyl) can be prepared according to the general method described by J. J. Parlow et al., J. Org. Chem. 1997, 62, 5908-5919 and modifications thereof as discussed for Scheme 8. 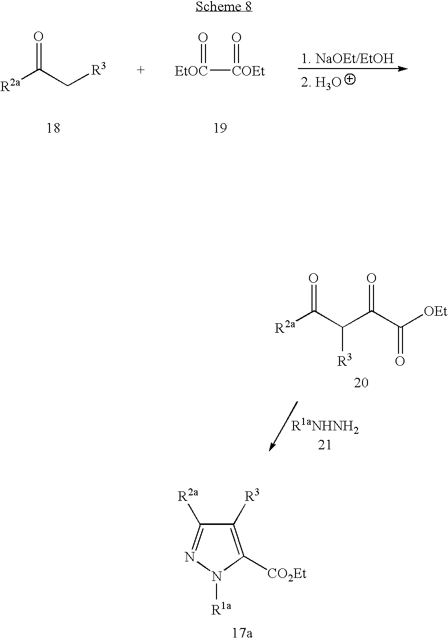
This method involves base-induced condensation of a ketone of Formula 18 with diethyl oxalate (19) to give a tricarbonyl compound of Formula 20, which is condensed with a hydrazine of Formula 21 to prepare the pyrazolecarboxylate of Formula 17a. The condensation of the tricarbonyl compound of Formula 20 with the hydrazine of Formula 21 is typically conducted in an alcohol, ester or carbonate diester solvent. The hydrazine of Formula 21 can be in the form of a salt. As a modification of the general method of Scheme 8, when R³ is H, the diketoester of Formula 20 can be alkylated or fluorinated to provide the corresponding diketoester of Formula 20 wherein R³ is alkyl or fluorine. The method of Scheme 8 is illustrated in Steps A and B of Example 1 and Steps A and B of Example 25.

As another modification of general method of Scheme 8, when R^{1a} is H, the pyrazolecarboxylate of Formula 17a can be alkylated with the appropriate alkylating agent in the presence of a base and solvent to give a pyrazolecarboxylate of Formula 17a wherein R^{1a} is alkyl, fluoroalkyl, alkenyl, fluoroalkenyl, alkynyl or fluoroalkynyl. Appropriate alkylating agents are typically of the formula R^{1a}X (22) wherein X is a nucleophilic reaction leaving group (e.g., bromide, iodide, mesylate (OS(O)₂CH₃), triflate (OS(O)₂CF₃), tosylate (OS(O)₂Ph-4-CH₃), etc.). Typical bases include potassium tert-butoxide, potassium carbonate, sodium hydride and potassium hydroxide. Typical solvents include N,N-dimethylformamide, acetonitrile and tetrahydrofuran. A particularly useful combination of base and solvent is potassium carbonate in acetonitrile. Alkylation isomers can be separated by common methods such as chromatography and crystallization. This modification is illustrated in Step C of Example 1 and Step C of Example 25.

Also, some of the R^{1a} groups can be converted to others on compounds of Formula 17a. For example, when R^{1a} is 2-hydroxyethyl, treatment with DAST (diethylaminosulfur trifluoride) typically gives a mixture of 2-fluoroethyl and vinyl for R^{1a}. The product compounds of Formula 17a wherein R^{1a} is 2-fluoroethyl and vinyl can then be separated by methods known in the art such as chromatography on silica gel and crystallization.

Compounds of Formula 18 are commercially available or can be prepared by methods well known in the art. For example, compounds of Formula 18 wherein R^{2a} is —CR²⁰(OR²¹)(OR²²) can be prepared according to the general procedure described by B. Tellegen, Recl. Trav. Chim. Pays-Bas 1938, 57, 133-141. Alternate approaches to construct R^{2a} using variations of the process of Scheme 8 are feasible. For example, a compound of Formula 17a wherein R^{2a} is a 1,1-dimethyl-2-haloethyl group can be prepared by first including R^{2a} in Formula 18 as a 1,1-dimethyl-2-hydroxyethyl group protected as a tetrahydropyranyl ether (e.g., prepared from dihydropyran and pyridinyl p-tosylate (PPTS) using the general procedure of M. Miyashita et al., J. Org. Chem. 1977, 142(23), 3772-3774), and then after preparation of the pyrazole ring according to the process of Scheme 8, deprotecting using PPTS to give the corresponding alcohol, which can then be converted to the mesylate using methanesulfonyl chloride and base, which is then displaced using an appropriate inorganic halide salt in N,N-dimethylformamide according to the general methods disclosed by P. Sulmon et al., Organic Preparations and Procedures Int. 1989, 21(1), 91-104 and European Patent EP-25,948-B1. Similarly, substituents can be completed after conducting the processes of other Schemes described herein as an alternative to including the substituents in final form in the starting materials for the processes.

Carboxylic esters of Formula 17b (Formula 17 wherein J is J-2 and R³¹ is ethyl) and Formula 17c (Formula 17 wherein J is J-3 and R³¹ is ethyl) wherein R^{1b} is alkyl, fluoroalkyl, alkenyl, fluoroalkenyl, alkynyl or fluoroalkynyl can be prepared from sydnones of Formula 23 and alkynes of Formula 24 according to the general method of J. Heterocycl. Chem. 1993, 30, 365-371 and J. Heterocycl. Chem. 1996, 33, 719-726 as depicted in Scheme 9. (One skilled in the art recognizes that to prepare 17b without a substituent at the pyrazole 5-position as specified for Formula 17b (J-2), the R³ radical in the sydnone of Formula 23 must be hydrogen.) 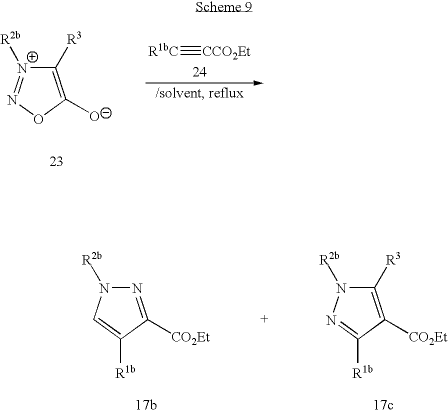
In this method, sydnones of Formula 23 are heated with alkynes of Formula 24 in higher boiling solvents (e.g., xylenes, toluene, dioxane, ethylene glycol) for typically 12-72 hours. The isomers 17b and 17c then can be separated by the usual methods such as column chromatography and distillation. The sydnones of Formula 23 can be prepared using the general methods described in J. Heterocycl. Chem. 1993, 30, 365-371, J. Heterocycl. Chem. 1996, 33, 719-726 and the references cited therein. The method of Scheme 9 is illustrated in Step A of Example 12 and Step A of Example 14.

Carboxylic esters of Formula 17d (Formula 17 wherein J is J-3 but R^{2c} can be H as well as R^{2b}; R³ is H and R³¹ is ethyl) wherein R^{1b} is alkyl, fluoroalkyl, alkenyl, fluoroalkenyl, alkynyl or fluoroalkynyl can also be prepared according to the method depicted in Scheme 10 wherein R³² is NMe₂ or OEt when (MeO)₂CHNMe₂ or HC(OEt)₃, respectively, is used to prepare intermediate 26. 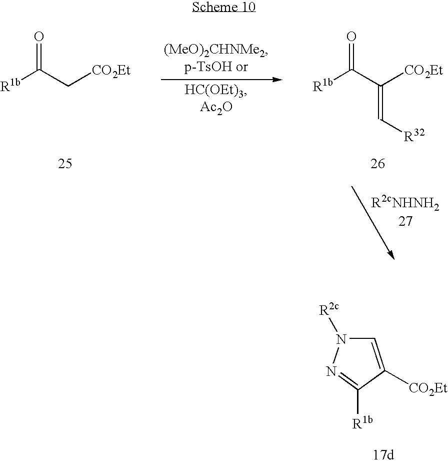
In this method the intermediate of Formula 26 is prepared from the ketoester of Formula 25 according to the general procedures published in J. Heterocycl. Chem., 1987, 24, 693-695. The starting ketoesters of Formula 25 can, in turn, be prepared according to the general procedures of J. Org. Chem. 1997, 62, 5908-5919. The condensation of the ketoester of Formula 26 with the hydrazine of Formula 27 is typically conducted in an alcohol, ester or carbonate diester solvent. The hydrazine of Formula 27 can be in the form of a salt.

When R^{2c} is H, the pyrazolecarboxylate of Formula 17d can be alkylated with the appropriate alkylating agent in the presence of a base and solvent to give a pyrazolecarboxylate of Formula 17d wherein R^{2c} is R^{2b}. Appropriate alkylating agents are typically of the formula R^{2b}X (28) wherein X is a nucleophilic reaction leaving group (e.g., bromide, iodide, mesylate (OS(O)₂CH₃), triflate (OS(O)₂CF₃), tosylate (OS(O)₂Ph-4-CH₃), etc.). Typical bases include potassium tert-butoxide, potassium carbonate, sodium hydride and potassium hydroxide. Typical solvents include N,N-dimethylformamide, acetonitrile and tetrahydrofuran. Alkylation isomers can be separated by common methods such as chromatography and crystallization.

Compounds of Formula 17b (i.e. pyrazole isomer J-2) can also be prepared using methods or slight modification thereof taught in: J. Heterocycl. Chem. 1999, 36(1), 217-220, Agric. Biol. Chem. 1984, 48(1), 45-50, Bull. Soc. Chim. Fr. 1978, (7-8, Pt. 2), 401-14, Khim. Geterotsikl. Soedin. 1968, 4(4), 685-94, European Patent Application Publication EP 419917 and Spanish Patent ES 493459 (1981). Compounds of Formula 17c (i.e. pyrazole isomer J-3) can also be prepared using methods or slight modification thereof taught in: J. Heterocycl. Chem. 1991, 28(6), 1545-7, J. Heterocycl. Chem. 1987, 24(6), 1669-75, J. Chem. Res., Synop. 1986, (5), 166-7, Aust. J. Chem. 1983, 36(1), 135-47, Japanese Patent Application Publications JP01061463, JP01106866, JP01061463 and JP 04021671, and Japanese Patents JP 2000212166 and JP 2000044541.

As shown in Scheme 11, pyrazoles of Formulae 17b and 17c (wherein R^{1b} is halogen) can be prepared from corresponding pyrazoles of Formula 17e (Formula 7 wherein J is J-2 but R^{1b} is H; and R³¹ is ethyl) and Formula 17f (Formula 17 wherein J is J-3 but R^{1b} is H; and R³¹ is ethyl), respectively. 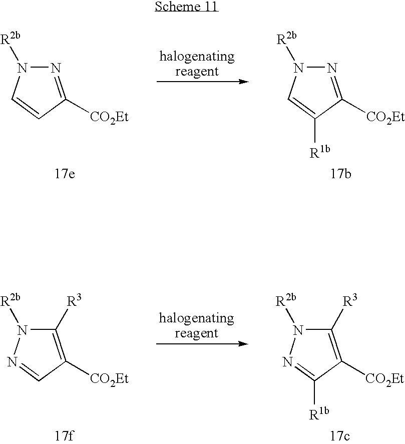
One variation of method of Scheme 11 involves heating a compound of Formula 17e or 17f with N-chloro- or N-bromosuccinimide in an organic solvent such as N,N-dimethyl-formamide, at temperatures between 30 and 110° C., preferably at about 60° C. Alternatively, bromine or chlorine can be added at or below room temperature to a compound of Formula 17e or 17f in a halocarbon solvent such as dichloromethane, trichloromethane or tetrachloromethane to give the corresponding compound of Formula 17b or 17c, respectively. The method of Scheme 11 is illustrated in Step B of Example 14.

Pyrazoles of Formula 17b and 17c wherein R^{1b} is halogen can also be prepared using the general methods taught in: Bulletin of the Korean Chemical Society 1998, 19(7), 725-726, Izv. Akad. Nauk SSSR, Ser. Khim. 1981, (6), 1342-8, Izv. Akad. Nauk SSSR, Ser. Khim. 1980, (5), 1071-7, J. Heterocycl. Chem. 1997, 34(2), 537-540, J. Heterocycl. Chem. 1991, 28(8), 1849-52, J. Fluorine Chem. 1988, 39(3), 435-40, U.S. Pat. No. 5,201,938, German Patent Application Publication DE 19632945-A1, and Japanese Patent Application Publications JP 10114750, JP 06056793, JP 05339242, JP 05043553, JP 03133961 and JP 01029364.

Thiazolecarboxylates of Formula 17 g (Formula 17 wherein J is J-4) can be prepared as illustrated in Scheme 12. 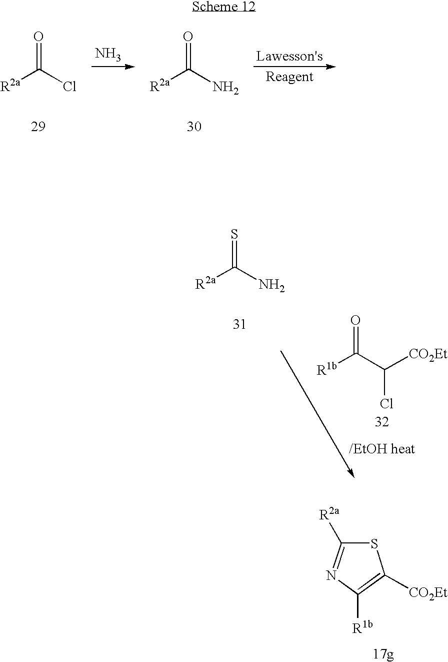
This method starts with an acyl chloride of Formula 29, which can be prepared by a variety of general methods known in the art; many acyl chlorides of Formula 29 are commercially available. The acyl chloride of Formula 29 is treated with an ammonia solution to prepare the carboxamide of Formula 30, which is in turn treated with a thionating reagent such as Lawesson's Reagent (2,4-bis(methoxyphenyl)-1,3-dithia-2,4-diphosphetane-2,4-disulfide) to prepare the thioamide of Formula 31. The thioamide of Formula 31 is then reacted with the chloro compound of Formula 32 to provide the thiazolecarboxylate of Formula 17 g.

Carboxylic esters of Formula 17h (Formula 17 wherein J is J-5) can be prepared by the general method shown in Scheme 13. 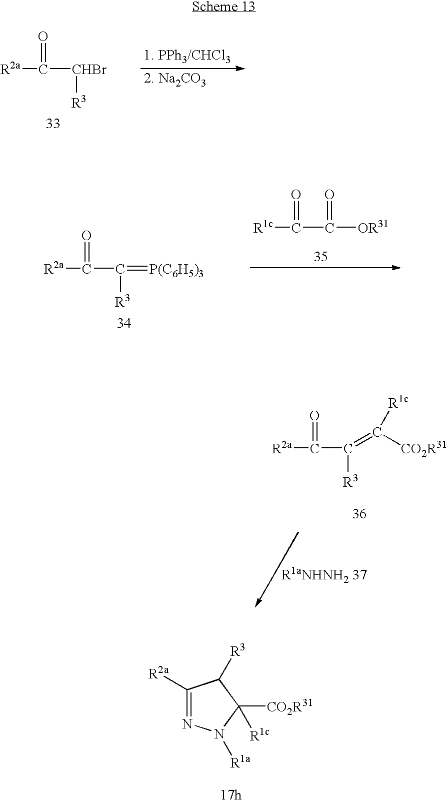
In this method, an alpha-bromo ketone of Formula 33 is converted to a Wittig reagent of Formula 34 and then condensed with a 2-oxocarboxylic acid ester of Formula 35 to provide a 4-oxo-2-pentenoic ester of Formula 36 according to the general procedure of P. F. Schuda et al., Synthesis 1987 (12), 1055-7. The 4-oxo-2-pentenoic ester of Formula 36 is then condensed with a hydrazine of Formula 37 to form the carboxylic ester of Formula 17h according to the general procedures of G. Westphal & H. H. Stroh, Liebigs Ann. Chem. 1968, 716, 160-163 and R. C. Moreau & P. Loiseau, Annales Pharmaceutiques Francaises 1978, 36 (1-2), 67-75. This method is illustrated by Steps A through C of Example 22.

Carboxylic esters of Formula 17i (Formula 17 wherein J is J-6 and R³¹ is ethyl) wherein R^{1d} is H, alkyl, fluoroalkyl, alkenyl, fluoroalkenyl, alkynyl or fluoroalkynyl can be prepared from sydnones of Formula 23 and alkenes of Formula 38 according to the general methods described in Z. Obshch. Khim. 1962, 32(5), 1446-1451 as depicted in Scheme 14.

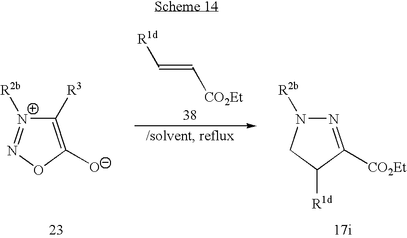

In this method, sydnones of Formula 23 are heated with alkenes of Formula 38 in higher boiling solvents (e.g., xylenes, toluene, dioxane, ethylene glycol) for typically 12-72 hours. The isomer 17i can then be isolated by the usual methods such as column chromatography and distillation.

The ester of Formula 17i can then be converted to the corresponding carboxylic acid as described for Scheme 7 and coupled to form the compound of Formula Ia as described for Schemes 1 and 2. Most R^{1b} substituents can be introduced as R^{1d} in the method of Scheme 14, but halogen cannot. Halogen as well as other R^{1b} substituents can be introduced in the method shown in Scheme 15. 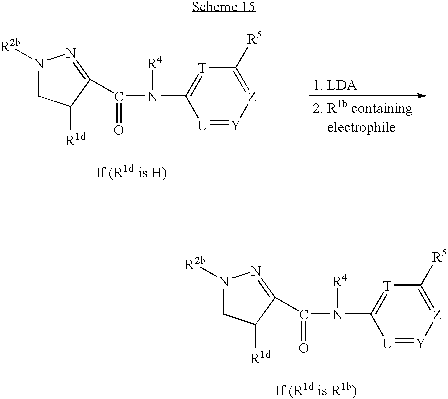
In this method, the compound of Formula If wherein R^{1d} is R^{1b} is prepared from the compound of Formula If wherein R^{1d} is H. The compound of Formula If wherein R^{1d} is H is then deprotonated using a strong base such as lithium diisopropylamide (LDA) and then reacted with an electrophile introducing R^{1b}. This general method is discussed by T. M. Stevenson et al., “1-Arylpyrazoline-3-carboxanilides” in Synthesis and Chemistry of Agrochemicals IV (D. R. Baker et al., Eds., American Chemical Society, Washington, D.C., 1995) Chapter 26, pp. 291-299. For halogenation, the electrophile can be elemental halogen (e.g., Cl₂, Br₂) or a halogen derivative such as N-bromosuccinimide or N-chlorosuccinimide. When R^{1b} is alkyl, fluoroalkyl, alkenyl, fluoroalkenyl, alkynyl or fluoroalkynyl the electrophile is typically of the formula R^{1b}X (39) wherein X is a nucleophilic reaction leaving group as already described for the compound of Formula 22 in connection with the modified method of Scheme 8.

Carboxylic esters of Formula 17j (Formula 17 wherein J is J-7) wherein R^{l}C is H can be prepared by the general method shown in Scheme 16. 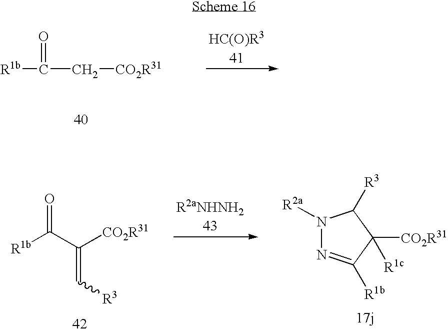
In this method, a 3-oxo-carboxylic acid ester of Formula 40 is condensed with an aldehyde of Formula 41 to provide an unsaturated ester of Formula 42, which is condensed with a hydrazine of Formula 43 to provide the carboxylic ester of Formula 17j according to the general procedure of P. S. Engel et al., J. Am. Chem. Soc. 1997, 119 (26), 6059-6065.

The ester of Formula 17j can then be converted to the corresponding carboxylic acid as described for Scheme 7 and coupled to form the compound of Formula Ia as described for Schemes 1 and 2. Alternatively as shown in Scheme 17, the coupling can be conducted first to prepare the amide of Formula 44, which is then condensed with the aldehyde of Formula 41 to prepare the unsaturated amide of Formula 45, which is condensed with the hydrazine of Formula 43 to prepare the compound of Formula Ig wherein R^{lc} is H. 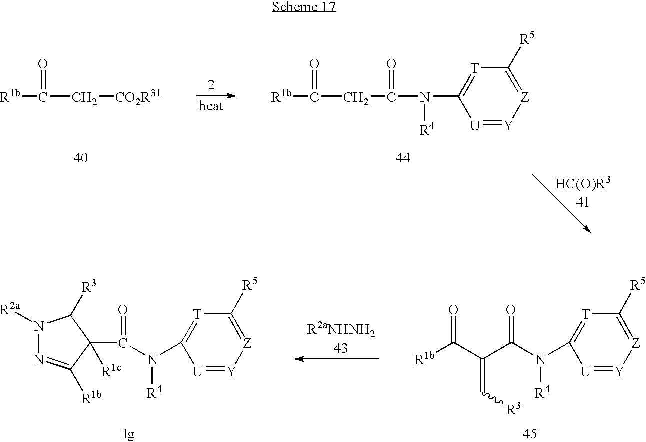
The method of Scheme 17 is illustrated in Steps A and B of Example 24.

Carboxylic esters of Formula 17k (Formula 17 wherein J is J-8) can be prepared by the general method shown in Scheme 18. 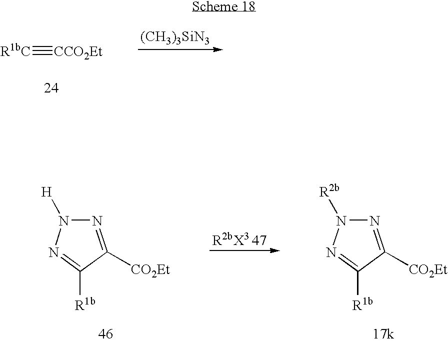
In this method, an alkynecarboxylic acid ester of Formula 24 is heated with an excess of azidotrimethylsilane at a temperature of about 100-110° C. under an inert atmosphere. The reaction is worked up by treating the cooled reaction mixture with excess methanol to consume remaining trimethylsilyl azide and desilylate the azide adduct. Evaporation leaves the 1,2,3-triazole of Formula 46. These conditions are described by R. S. Klein et al., J. Heterocycl. Chem. 1976, 13, 589-592 and illustrated by Step A of Example 26. The triazole of Formula 46 is then converted to the triazole of Formula 17k by alkylation with R^{2b}X³ (47) wherein X³ is a nucleophilic reaction leaving group such as Cl, Br, I, sulfonates such as p-toluenesulfonate, methanesulfonate or trifluoromethanesulfonate, or sulfates such as —OSO₂OR^{2b}. Preferably, X³ is a strong leaving groups such as I. The reaction is conducted in the presence of a base such as potassium carbonate in a polar aprotic solvent such as acetonitrile at a temperature commonly between 40 and 80° C., typically about 50-60° C. Filtration to remove solid byproducts and evaporation of the solvent leaves a crude product containing the triazole of Formula 17k typically together with other alkylation regioisomers. The triazole of Formula 17k can be isolated and purified by the usual methods known to those skilled in the art such as chromatography and crystallization. This method is illustrated by Step B of Example 26.

When R^{2b} is a tertiary alkyl group such as tert-butyl, alkylation with R^{2b}X³ may give low yields. Compounds of Formula 17k wherein R^{2b} is a tertiary alkyl group can be satisfactorily prepared from compounds of Formula 46 by reaction with the appropriate alcohol R^{2b}OH (48) in trifluoroacetic acid solution in the presence of concentrated sulfuric acid according to the general procedure of J. W. Tilley et al., J. Med. Chem. 1991, 34(3), 1125-1134. This method is illustrated by Step A of Example 28.

Scheme 19 describes another method for preparing carboxylic ester intermediates of Formula 17k (Formula 17 wherein J is J-8).

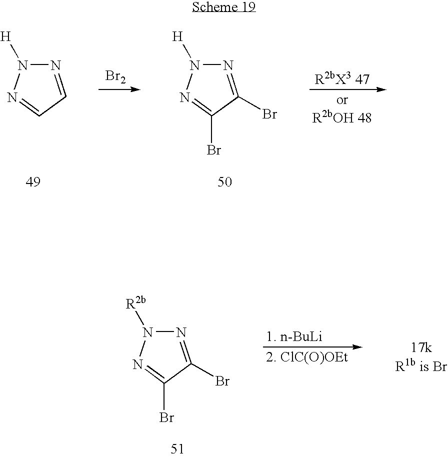

According to the method of B. Iddon and M. Nicholas, J. Chem. Soc., Perkin Trans. 1 1996, 1341-1347, bromine is added to an aqueous solution of 1,2,3-triazole (49), and 4,5-dibromo-1,2,3-triazole (50) is collected by filtration. This is then alkylated with R^{2b} using either an alkylating agent of Formula 47 or an alcohol of Formula 48 to provide the compound of Formula 51 using methods analogous to those already described for conversion of Formula 46 to Formula 17k in Scheme 18. Following the general method of B. Iddon and M. Nicholas, J. Chem. Soc., Perkin Trans. 1 1996, 1341-1347, the compound of Formula 51 is lithiated using n-butyllithium in an ether solvent such as ethyl ether or tetrahydrofuran at −70 to −100° C., optionally magnesium bromide is added, followed by ethyl chloroformate to give the compound of Formula 17k where R^{1b} is Br. Lithiation using n-butyllithium in tetrahydrofuran at −78° C., followed by addition of ethyl chloroformate works well. The compound of Formula 17k where R^{1b} is Br is useful for preparing compounds of Formula I where J is J-8 and R^{1b} is Br. Furthermore, Br can be replaced by other R^{1b} groups such as vinyl by a variety of coupling methods known in the art. For example, the bromine can be replaced by a 1-alkenyl group through mediation of a palladium catalyst in the Heck Reaction (for reviews, see R. A. Abramovitch et al., Tetrahedron 1988, 44(11), 3039-3071; W. Cabri and I. Candiani, Synthesis 1995, 28(1), 2-7; and R. F. Heck, “Palladium-catalyzed Vinylation of Organic Halides”, Chapter 2 in Organic Reactions, Vol. 27, Wiley: New York, 1982, pp. 345-390). The Heck Reaction is compatible with some fluoroalkenes, such as 3,3,3-trifluoropropene; see G. Meazza et al., Pestic. Sci. 1992, 35, 137-144. Furthermore, compounds of Formula 17k where R^{1b} is Br can be reacted with alkenyl- and alkynyl-stannanes to afford alkenyl and alkynyl groups, respectively, as R^{1b} by use of the Stille Reaction, as reviewed by V. Farina et al., “The Stille Reaction”, Chapter 1 in Organic Reactions, Vol. 50, Wiley: New York, 1997, pp. 1-652.

Although ethyl esters are shown for the compounds of Formulae 24, 46 and 17k, one skilled in the art recognizes that corresponding esters wherein ethyl is replaced by other carbon-based radicals, e.g., R³¹, can be used as well for this method. Also known in the art are other methods to prepare 1,2,3-triazole rings, such as those described in PCT Patent Publication WO 02/096258.

Carboxylic esters of Formula 171 (Formula 17 wherein J is J-9) can be prepared by the general method shown in Scheme 20. 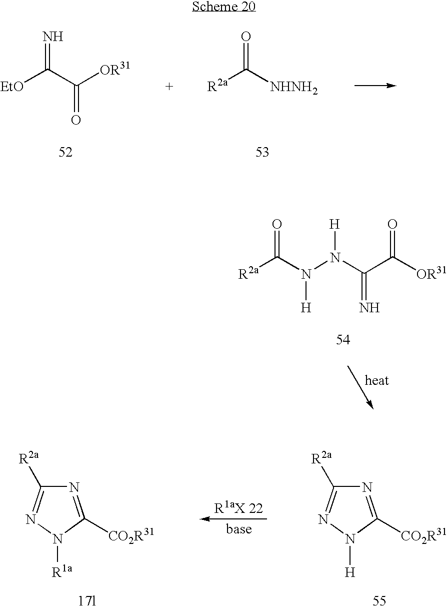
In this method, a iminoacetate of Formula 52 is reacted with a carboxylic acid hydrazide of Formula 53 in a suitable solvent such as dichloromethane to give the adduct of Formula 54. Although the reaction can be conducted a higher temperatures, it typically proceeds at a useful rate even at room temperature. The compound of Formula 54 is then cyclized to give the triazole of Formula 55 by heating to a sufficiently high temperature, typically around 200° C. Although the reaction can be conducted using a high boiling solvent, most conveniently it is done in the absence of solvent. The triazole of Formula 55 is then alkylated using R^{1a}X (22) in the presence of a base and solvent, analogous to the alkylation of pyrazoles already described as a modification of the method of Scheme 8. This method is further illustrated by Steps A and B of Example 30 below.

Amino compounds of Formula 2 can be prepared by a wide variety of methods available to the synthetic organic chemist. Many of these methods involve converting one substituent to another on the aromatic ring. For example, the amino function of Formula 2a (Formula 2 wherein R⁴ is H, T is CR⁶, U is CR⁷, Y is CR⁸ and Z is CR⁹) can be obtained by reduction of the nitro compound of Formula 60 as shown in Scheme 21. 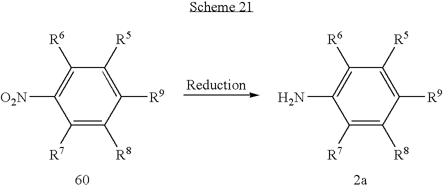
The nitro compound of Formula 60 can be reduced to the aniline of Formula 2a by a variety of reducing agents known in the art, such as iron in acetic acid, tin(II) chloride or hydrogenation over a palladium or platinum sulfide catalyst. The nitro function of Formula 60 can be added by well known nitration reactions. The method of Scheme 19 is illustrated in Step B of Example 4, Step C of Example 7, Step B of Example 16 and Step B of Example 17. Many compounds of Formula 60 are commercially available. When T, U and/or Z are N, the aryl ring of Formula 2 is activated to nucleophilic substitution facilitating introduction of amino by displacement of leaving groups such as halogen.

As another example of conversion of one substituent to another, compounds of Formula 2b (Formula 2 wherein R⁴ is H and R⁵ is CO₂R¹²) wherein T is CR⁶ or N; U is CR⁷ or N; Y is CR⁸ or N; Z is CR⁹ or N; R⁶, R⁷, R⁸ and R⁹ are each independently H or F; and R¹² is C₁-C₅ alkyl, C₂-C₅ haloalkyl, C₃-C₅ alkenyl, C₃-C₅ haloalkenyl, C₃-C₅ alkynyl, C₃-C₅ cycloalkyl or C₄-C₅ cycloalkylalkyl can be prepared as shown in Scheme 22. 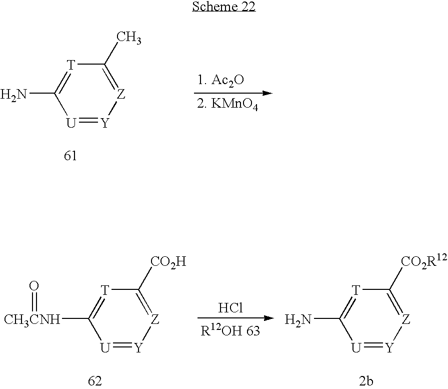
In the method of Scheme 22, the amino function of a compound of Formula 61 is protected as the acetamide by treatment with acetic anhydride. Treatment with potassium permanganate then oxidizes the aromatic methyl radical to a carboxylic acid function to provide the compound of Formula 62. The compound of Formula 62 is then treated with strong acid, such as hydrochloric acid and alcohol of Formula 63 to form the ester group and deprotect the amino radical. This method works particularly well for short aliphatic alcohols (e.g., R¹² is Me or Et). The method of Scheme 20 is illustrated in Steps A through C of Example 8 and Steps A through C of Example 10. Other synthetic approaches to prepare compounds of Formula 2b are also feasible, as is illustrated by Steps A through C of Example 19. Compounds of Formula 2b wherein R¹² is methyl or ethyl can be coupled to form compounds of Formula Ia wherein R⁴ is H and R⁵ is CO₂R¹² according to the methods of Schemes 1 and 2, and then R¹² converted to other radicals or CO₂R¹² converted to other groups such C(O)NR¹⁰R¹¹ according to the method of Scheme 4 and other methods known to those skilled in the art. This conversion is illustrated by Example 20.

As still another example of conversion of one substituent to another, amides of Formula 2 wherein R⁵ is C(O)NR¹⁰R¹¹ can be converted to thioamides of Formula 2 wherein R⁵ is C(S)NR¹⁰R¹¹ using the thionating reagents already described for the method of Scheme 3.

It is recognized that some reagents and reaction conditions described above for preparing compounds of Formula I or Iz may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, T. W. Greene, P. G. M. Wuts, Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula I or Iz. One skilled in the art will also recognize that it may be necessary to perform a combination of the steps illustrated in the above schemes in an order other than that implied by the particular sequence presented to prepare the compounds of Formula I or Iz.

One skilled in the art will also recognize that compounds of Formula I (or Iz) and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; ¹⁹F NMR spectra are reported in ppm relative to CF₃CCl₃; “s” means singlet, “d” means doublet, “t” means triplet, “q” means quartet, “m” means multiplet, “dd” means doublet of doublets, “dt” means doublet of triplets, “dq” means doublet of quartets, “br s” means broad singlet, “br d” means broad doublet.

### EXAMPLE 1

### Preparation of ethyl 3-[[[3-(1,1-dimethylethyl)-1-ethyl-1H-pyrazol-5-yl]carbonyl]-amino]benzoate (Compound 80)

### Step A: Preparation of ethyl 2-hydroxy-5,5-dimethyl-4-oxo-2-hexenoate

To a solution of sodium ethoxide in ethanol (250 mL, 21% by weight in ethanol, 670 mmol) was added dropwise a solution of diethyl oxalate (45.2 mL, 332.5 mmol) and pinacolone (alternatively named 3,3-dimethyl-2-butanone) (41.7 mL) in ethanol (300 mL) at room temperature under nitrogen atmosphere. The reaction mixture was stirred at room temperature overnight, concentrated to its half volume and poured into ice. Concentrated hydrochloric acid was added to lower the pH to approximately 4, and then the mixture was extracted with ethyl acetate. The extracts were dried over magnesium sulfate and concentrated to give the title compound as an oil (60 g, yield 90%).

### Step B: Preparation of ethyl 5-(1,1-dimethylethyl)-1H-pyrazole-3-carboxylate

To a solution of ethyl 2-hydroxy-5,5-dimethyl-4-oxo-2-hexenoate (i.e. the product of Step A) (45.3 g, 226 mmol) in ethanol (200 mL) and acetic acid (2 mL) was added hydrazine monohydrate (12.1 mL, 249 mmol) dropwise under nitrogen atmosphere at room temperature. The reaction mixture was stirred at room temperature overnight and concentrated to give 40.8 g of the title compound.

¹H NMR (CDCl₃) δ 6.7 (s, 1H), 6.60 (br s, 1H), 4.40 (q, 2H), 1.40 (t, 3H), 1.32 (s, 9H).

### Step C: Preparation of ethyl 3-(1,1-dimethylethyl)-1-ethyl-1H-pyrazole-5-carboxylate

To a solution of ethyl 5-(1,1-dimethylethyl)-1H-pyrazole-3-carboxylate (i.e. the product of Step B) (20.0 g, 102 mmol) in anhydrous NN-dimethylformamide (DMF) (100 mL) was added sequentially potassium carbonate (28.2 g, 204 mmol) and iodoethane (11.4 mL, 143 mmol) at room temperature. After stirring at room temperature in an inert atmosphere for 6 h, the reaction mixture was diluted with ethyl acetate (400 mL) and washed with water (2×50 mL). The organic phase was separated, dried and concentrated. The residue was purified by chromatography on silica gel to give the desired isomer (i.e. the title compound) as a white solid (13.8 g, 64% yield) and a minor isomer (2.1 g, 10% yield).

¹H NMR (CDCl₃) δ 6.7 (s, 1H), 4.55 (q, 2H), 4.32 (q, 2H), 1.40 (m, 6H), 1.32 (s, 9H).

¹H NMR (CDCl₃) (minor isomer) δ 6.7 (s, 1H), 4.20 (q, 2H), 4.30 (q, 2H), 1.36 (m, 6H), 1.32 (s, 9H).

### Step D: Preparation of 3-(1,1-dimethylethyl)-1-ethyl-1H-pyrazole-5-carboxylic acid

A solution of ethyl 3-(1,1-dimethylethyl)-1-ethyl-1H-pyrazole-5-carboxylate (i.e. the product of Step C) (6.9 g, 30.8 mmol) in ethanol (200 mL) was stirred with an aqueous solution of sodium hydroxide (10%, 19 mL) at room temperature for 6 h. The mixture was then concentrated and acidified with 1 N hydrochloric acid. The precipitated solids were filtered and dried to give 6 g of the title acid as a white solid.

¹H NMR (CDCl₃) δ 10.00 (s, 1H), 6.80 (s, 1H), 4.60 (q, 2H), 1.40 (t, 3H), 1.32 (s, 9H).

### Step E: Preparation of 3-(1,1-dimethylethyl)-1-ethyl-1H-pyrazole-5-carbonyl chloride

A solution of 3-(1,1-dimethylethyl)-1-ethyl-1H-pyrazole-5-carboxylic acid (i.e. the product of Step D) (1.2 g, 6.11 mmol) and oxalyl chloride (2 mL) in dichloromethane (30 mL) in the presence of anhydrous DMF (0.1 mL) was stirred under nitrogen atmosphere at room temperature for 4 h. The reaction mixture was then concentrated to yield the title acid chloride as a liquid.

### Step F: Preparation of ethyl 3-[[[3-(1,1-dimethylethyl)-1-ethyl-1H-pyrazol-5-yl]-carbonyl]amino]benzoate

A solution of 3-(1,1-dimethylethyl)-1-ethyl-1H-pyrazole-5-carbonyl chloride (i.e. the product of Step E) (1.3 g) in dichloromethane (30 mL) was added to a solution of ethyl 3-aminobenzoate (1.21 g) in dichloromethane (10 mL) in the presence of triethylamine (2 mL) and 4-(dimethylamino)pyridine (DMAP) (0.1 g). After stirring at room temperature overnight the reaction mixture was diluted with dichloromethane (50 mL) and washed with 1 N hydrochloric acid. The organic phase was separated, dried (MgSO₄) and concentrated. The residue was purified by chromatography on silica gel to give the title compound (1.7 g, 81% yield), a compound of present invention, as a solid.

¹H NMR (CDCl₃) δ 8.01 (m, 2H), 7.80 (d, 1H), 7.42 (t, 1H), 6.53 (s, 1H), 4.57 (q, 2H), 4.38 (q, 2H), 1.38 (m, 6H), 1.34 (s, 9H).

### EXAMPLE 2

### Preparation of 2-fluoroethyl 3-[[[3-(1,1-dimethylethyl)-1-ethyl-1H-pyrazol-5-yl]-carbonyl]amino]benzoate (Compound 82)

### Step A: Preparation of 3-[[[3-(1,1-dimethylethyl)-1-ethyl-1H-pyrazol-5-yl]carbonyl]-amino]benzoic acid

A solution of ethyl 3-[[[3-(1,1-dimethylethyl)-1-ethyl-1H-pyrazol-5-yl]carbonyl]-amino]benzoate (i.e. the product of Example 1, Step F) (4.8 g, 14 mmol) in methanol (30 mL) was stirred with an aqueous solution of sodium hydroxide (10%, 17 mL) at room temperature for 6 h. The reaction mixture was then concentrated and acidified with 1 N hydrochloric acid. The precipitated solids were filtered and dried to give the title acid as a white solid (4.3 g).

¹H NMR (CDCl₃) δ 10.6 (s, 1H), 8.38 (s, 1H), 8.00 (d, 1H), 7.62 (d, 1H), 7.40 (t, 1H), 4.47 (q, 2H), 1.34 (t, 3H), 1.20 (s, 9H).

### Step B: Preparation of 3-[[[3-(1,1-dimethylethyl)-1-ethyl-1H-pyrazol-5-yl]carbonyl]-amino]benzoyl chloride

A solution of the 3-[[[3-(1,1-dimethylethyl)-1-ethyl-1H-pyrazol-5-yl]carbonyl]amino]-benzoic acid (i.e. the product of Step A) (1.2 g, 3.80 mmol), oxalyl chloride (1.72 mL) and anhydrous DMF (0.1 mL) in dichloromethane (10 mL) was stirred under nitrogen atmosphere at room temperature for 4 h. The reaction mixture was then concentrated to yield the title acid chloride.

### Step C: Preparation of 2-fluoroethyl 3-[[[3-(1,1-dimethylethyl)-1-ethyl-1H-pyrazol-5-yl]carbonyl]amino]benzoate

To a solution of the 3-[[[3-(1,1-dimethylethyl)-1-ethyl-1H-pyrazol-5-yl]carbonyl]-amino]benzoyl chloride (i.e. the product of Step B) (0.2 g) in dichloromethane (5 mL) was added a solution of 2-fluoroethanol (0.1 mL), triethylamine (0.2 mL) and DMAP (20 mg) under nitrogen atmosphere at room temperature. After stirring at room temperature for 6 h, the reaction mixture was diluted with dichloromethane (15 mL) and washed with 1 N hydrochloric acid (5 mL). The organic phase was dried and concentrated. The residue was purified by chromatography on silica gel to give the title compound (155 mg), a compound of present invention.

¹H NMR (CDCl₃) δ 8.05 (m, 1H), 7.88 (d, 1H), 7.70 (br s, 1H, NH), 7.42 (t, 1H), 6.50 (s, 1H), 4.60 (m, 6H), 1.42 (t, 3H), 1.34 (s, 9H).

### EXAMPLE 3

### Preparation of 3-(1,1-dimethylethyl)-1-ethyl-N-[3-[[(2,2,2-trifluoroethyl)amino]carbonyl]-phenyl]-1H-pyrazole-5-carboxamide (Compound 43)

To a solution of 3-[[[3-(1,1-dimethylethyl)-1-ethyl-1H-pyrazol-5-yl]carbonyl]amino]-benzoyl chloride (i.e. the product of Example 2, Step B) (0.2 g) in dichloromethane (5 mL) was added sequentially 2,2,2-trifluoroethylamine (0.1 mL), triethylamine (0.2 mL) and DMAP (20 mg) at room temperature. After stirring at room temperature for 6 h, the reaction mixture was diluted with dichloromethane (15 mL) and washed with hydrochloric acid (1 N, 5 mL). The organic phase was separated, dried and concentrated. The residue was purified by chromatography on silica gel to give the title compound (155 mg), a compound of present invention.

¹H NMR (CDCl₃) δ 7.44 (m, 3H), 7.12 (dd, 1H), 6.76 (s, 1H), 6.42 (s, 1H, NH), 4.60 (q, 2H), 4.12 (m, 2H), 1.42 (t, 3H), 1.38 (s, 9H).

### EXAMPLE 4

### Preparation of ethyl 3-[[[3-(1,1-dimethylethyl)-1-ethyl-1H-pyrazol-5-yl]carbonyl]amino-4-fluorobenzoate (Compound 70)

### Step A: Preparation of ethyl 4-fluoro-3-nitrobenzoate

A mixture of 4-fluoro-3-nitrobenzoic acid (10 g, 54 mmol), diethyl sulfate (8.5 mL) and potassium carbonate (10 g) in anhydrous acetone (120 mL) was heated to reflux for 6 h. The reaction mixture was then filtered, and the filtrate was concentrated. The residue was purified by chromatography on silica gel to give the title compound (11.2 g) as a yellow oil.

¹H NMR (CDCl₃) δ 8.64 (dd, 1H), 8.32 (m, 1H), 7.38 (t, 1H), 4.44 (q, 2H), 1.40 (t, 3H).

### Step B: Preparation of ethyl 3-amino-4-fluorobenzoate

A solution of ethyl 4-fluoro-3-nitrobenzoate (the product of Step A) (5.7 g, 26.7 mmol) in acetic acid (50 mL) and ethyl acetate (60 mL) was added dropwise over 20 minutes to a suspension of iron powder (6.0 g) in acetic acid (5% wt, 30 mL) at 80° C. After the addition, the reaction mixture was stirred at 80° C. for additional 20 minutes. The mixture was then cooled to room temperature. Solids were removed by filteration through Celite® diatomaceous filter aid, and the filtrate was concentrated. The residue was diluted with ethyl acetate (100 mL) and washed sequentially with water (25 mL) and aqueous sodium bicarbonate solution (5%, 25 mL). The organic layer was dried and concentrated to give the title compound (4.5 g).

¹H NMR (CDCl₃) δ 7.60 (dd, 1H), 7.42 (m, 1H), 7.08 (t, 1H), 4.34 (q, 2H), 3.90 (br s, 2H), 1.34 (t, 3H).

### Step C: Preparation of ethyl 3-[[[3-(1,1-dimethylethyl)-1-ethyl-1H-pyrazol-5-yl]-carbonyl]amino-4-fluorobenzoate

A solution of 3-(1,1-dimethylethyl)-1-ethyl-1H-pyrazole-5-carbonyl chloride (i.e. the product of Example 1, Step E) (4.7 g) in dichloromethane (40 mL) was added to a solution of ethyl 3-amino-4-fluorobenzoate (i.e. the product of Step B) (4.46 g, 24.3 mmol) and N,N-diisopropylethylamine (8.5 mL) in dichloromethane (10 mL). After stirring at room temperature overnight, the reaction mixture was diluted with dichloromethane (100 mL) and washed with 1 N hydrochloric acid. The organic phase was separated, dried and concentrated. The residue was purified by chromatography on silica gel to give the title compound (6.6 g), a compound of the present invention.

¹H NMR (CDCl₃) δ 8.14 (m, 1H), 8.00 (dd, 1H), 7.26 (s, 1H), 6.26 (s, 1H), 4.34 (m, 4H), 1.41 (m, 6H), 1.20 (s, 9H).

### EXAMPLE 5

### Preparation of 3-(1,1-dimethylethyl)-1-ethyl-N-[5-[(ethylamino)carbonyl]-2-fluorophenyl]-1H-pyrazole-5-carboxamide (Compound 2)

### Step A: Preparation of 3-[[[3-(1,1-dimethylethyl)-1-ethyl-1H-pyrazol-5-yl]carbonyl]-amino]-4-fluorobenzoic acid

A solution of ethyl 3-[[[3-(1,1-dimethylethyl)-1-ethyl-1H-pyrazol-5-yl]carbonyl]-amino-4-fluorobenzoate (i.e. the product of Example 4, Step C) (6.6 g, 18.3 mmol) in methanol (40 mL) and aqueous sodium hydroxide (10%, 17 mL) was stirred at room temperature for 6 h. The reaction mixture was then concentrated and acidified with 1 N hydrochloric acid. The precipitated solids were filtered and dried to give 5.3 g of the title acid as a white solid.

¹H NMR (DMSO-d₆) δ 10.54 (s, 1H), 8.22 (dd, 1H), 7.86 (m, 1H), 7.40 (t, 1H), 6.89 (s, 1H), 4.44 (q, 2H), 1.32 (t, 3H), 1.30 (s, 9H).

### Step B: Preparation of 3-(1,1-dimethylethyl)-1-ethyl-N-[5-[(ethylamino)carbonyl]-2-fluorophenyl]-1H-pyrazole-5-carboxamide

To a solution of 3-[[[3-(1,1-dimethylethyl)-1-ethyl-1H-pyrazol-5-yl]carbonyl]amino]-4-fluorobenzoic acid (i.e. the product of Step A) (200 mg) in dichloromethane (5 mL) was added sequentially 1-propanephosphonic acid cyclic anhydride (50% in ethyl acetate, 4 mL), ethylamine (0.3 mL) and DMAP (0.2 g) at room temperature. After stirring at room temperature overnight, the reaction mixture was diluted with dichloromethane (10 mL) and washed with 1 N hydrochloric acid (5 mL). The organic phase was separated, dried and concentrated. The residue was purified by chromatography on silica gel to give the title compound, a compound of present invention, as white solid, m.p. 188.5° C.

### EXAMPLE 6

### Alternate Preparation of 3-(1,1-dimethylethyl)-1-ethyl-N-[5-[(ethylamino)carbonyl]-2-fluorophenyl]-1H-pyrazole-5-carboxamide (Compound 2)

To a solution of 3-[[[3-(1,1-dimethylethyl)-1-ethyl-1H-pyrazol-5-yl]carbonyl]amino]-4-fluorobenzoic acid (i.e. the product of Example 5, Step A) (200 mg) in dichloromethane (5 mL) was added oxalyl chloride (0.5 mL) and anhydrous DMF (0.1 mL). After stirring at room temperature for 2 h, the reaction mixture was concentrated under reduced pressure. To a solution of the residue (200 mg) in dichloromethane (5 mL) at room temperature was added sequentially ethylamine (0.3 mL), triethylamine (0.5 ml) and DMAP (0.1 g). After stirring at room temperature for 6 h, the reaction mixture was diluted with dichloromethane (10 mL) and washed with hydrochloric acid (1 N, 5 mL). The organic phase was separated, dried and concentrated. The residue was purified by chromatography on silica gel to give the title compound, a compound of present invention, as white solid, m.p. 188.5° C.

### EXAMPLE 7

### Preparation of N-[5-[(dimethylamino)carbonyl]-2-fluorophenyl]-3-(1,1-dimethylethyl)-1-ethyl-1H-pyrazole-5-carboxamide (Compound 6)

### Step A: Preparation of 4-fluoro-3-nitrobenzoyl chloride

A solution of 4-fluoro-3-nitrobenzoic acid (13 g; 70 mmol), oxalyl chloride (8.5 mL) and DMF (0.5 mL) in anhydrous dichloromethane (200 mL) was stirred at room temperature under nitrogen atmosphere for 2 h. The reaction mixture was then concentrated to remove the solvent, and the crude title compound was used for the next reaction without further purification (13 g).

### Step B: Preparation of 4-fluoro-N,N-dimethyl-3-nitrobenzamide

To a solution of 4-fluoro-3-nitrobenzoyl chloride (i.e. the product of Step A) (4.1 g) in dichloromethane (50 mL) was added dimethylamine hydrochloride (2.13 g) and N,N-diisopropylethylamine (4 mL) at room temperature. After stirring at room temperature for 6 h, the reaction mixture was diluted with dichloromethane (100 mL) and washed with 1 N hydrochloric acid (15 mL). The organic phase was separated, dried and concentrated. The residue was purified by chromatography on silica gel to give the title compound (3.4 g) as white solid.

### Step C: Preparation of 3-amino-4-fluoro-NN-dimethylbenzamide

A solution of 4-fluoro-N,N-dimethyl-3-nitrobenzamide (i.e. the product of Step B) (1.8 g, 8.5 mmol) in acetic acid (9 mL) and ethyl acetate (10 mL) was added dropwise over 20 minutes to a suspension of iron powder (1.5 g) in acetic acid (5%, 5 mL) at 80° C. After the addition, the reaction mixture was stirred at 80° C. for additional 20 minutes. The mixture was then cooled to room temperature. Solids were removed by filteration through Celite® diatomaceous filter aid, and the filtrate was concentrated. The residue was diluted with ethyl acetate (50 mL) and washed sequentially with water (10 mL) and aqueous sodium bicarbonate solution (5%, 15 mL). The organic layer was dried and concentrated to give the title compound (1.1 g).

### Step D: Preparation of N-[5-[(dimethylamino)carbonyl]-2-fluorophenyl]-3-(1,1-dimethylethyl)-1-ethyl-1H-pyrazole-5-carboxamide

A solution of 3-(1,1-dimethylethyl)-1-ethyl-1H-pyrazole-5-carbonyl chloride (i.e. the product of Example 1, Step E) (1.2 g) in dichloromethane (10 mL) was added to a solution of 3-amino-4-fluoro-N,N-dimethylbenzamide (i.e. the product of Step C) (1.1 g) and N,N-diisopropylethylamine (2.5 mL) in dichloromethane (5 mL). After stirring at room temperature for 6 h, the reaction mixture was diluted with dichloromethane (20 mL) and washed with 1 N hydrochloric acid. The organic phase was separated, dried and concentrated. The residue was purified by chromatography on silica gel to give the title compound (1.8 g), a compound of present invention.

¹H NMR (CDCl₃) δ 8.40 (dd, 1H), 8.02 (br s, 1H, NH), 7.22 (m, 2H), 6.54 (s, 1H), 4.58 (q, 2H), 3.10 (s, 3H), 3.03 (s, 3H), 1.44 (t, 3H), 1.34 (s, 9H).

### EXAMPLE 8

### Preparation of methyl 6-[[[3-(1,1-dimethylethyl)-1-ethyl-1H-pyrazol-5-yl]carbonyl]amino]-2-pyridinecarboxylate (Compound 143)

### Step A: Preparation of N-(6-methyl-2-pyridinyl)acetamide

A solution of 2-amino-6-picoline (20 g, 185 mmol) and acetic anhydride (35 mL) in anhydrous tetrahydrofuran (THF) (150 mL) was heated at reflux for 10 h. The reaction mixture was then cooled to room temperature and concentrated to leave a thick oily residue. The residue was dissolved in dichloromethane (400 mL) and washed sequentially with hydrochloric acid (1 N, 50 mL) and water (50 mL). The organic phase was dried and concentrated to give the title compound as a white solid (27.6 g, 99% yield).

¹H NMR (CDCl₃) δ 8.02 (d, 1H), 8.00 (br s, 1H, NH), 7.61 (t, 1H), 6.90 (d, 1H), 2.44 (s, 3H), 2.20 (s, 3H).

### Step B: Preparation of 6-(acetylamino)-2-pyridinecarboxylic acid

To a suspension of N-(6-methyl-2-pyridinyl)acetamide (i.e. the product of Step A) (27 g, 184 mmol) in water (250 mL) at 90° C. was added potassium permanganate (29.1 g, 184 mmol) in small portions. After the addition, the mixture was heated to 90° C. for 6 h. The mixture was then cooled and filtered through a pad of Celite® diatomaceous filter aid. The filtrate was concentrated to half of its volume and acidified with concentrated hydrochloric acid. The precipitated solids were isolated by filtration and dried to give 20 g of the title compound.

### Step C: Preparation of methyl 6-amino-2-pyridinecarboxylate

Hydrogen chloride gas was bubbled through a suspension of 6-(acetylamino)-2-pyridinecarboxylic acid (i.e. the product of Step B) (20 g) in methanol (100 mL) for 1 h. The reaction mixture was then heated to reflux overnight. Concentration followed by purification on silica gel column provided the title compound (12 g).

¹H NMR (CDCl₃) δ 7.52 (m, 2H), 6.69 (d, 1H), 4.80 (br s, 2H, NH2), 3.96 (s, 3H).

### Step D: Preparation of methyl 6-[[[3-(1,1-dimethylethyl)-1-ethyl-1H-pyrazol-5-yl]-carbonyl]amino]-2-pyridinecarboxylate

To a solution of 3-(1,1-dimethylethyl)-1-ethyl-1H-pyrazole-5-carbonyl chloride (i.e. the product of Example 1, Step E) (1.2 g) in dichloromethane (10 mL) was added sequentially a solution of methyl 6-amino-2-pyridinecarboxylate (i.e. the product of Step C) (1.03 g) in dichloromethane (5 mL) followed by triethylamine (2 mL) and then DMAP (0.1 g). After stirring at room temperature for 6 h, the reaction mixture was diluted with dichloromethane (20 mL) and washed with 1 N hydrochloric acid. The organic phase was separated, dried and concentrated. The residue was purified by chromatography on silica gel to give the title compound (1.24 g), a compound of the present invention.

¹H NMR (CDCl₃) δ 8.72 (s, 1H, NH), 8.42 (m, 1H), 7.82 (d, 2H), 6.64 (s, 1H), 4.48 (q, 2H), 4.01 (s, 3H), 1.45 (t, 3H), 1.31 (s, 9H).

### EXAMPLE 9

### Preparation of 6-[[[3-(1,1-dimethylethyl)-1-ethyl-1H-pyrazol-5-yl]carbonyl]amino]-2-pyridinecarboxamide (Compound 162)

### Step A: Preparation of 6-[[[3-(1,1-dimethylethyl)-1-ethyl-1H-pyrazol-5-yl]carbonyl]-amino-2-pyridinecarboxylic acid

A solution of methyl 6-[[[3-(1,1-dimethylethyl)-1-ethyl-1H-pyrazol-5-yl]carbonyl]-amino]-2-pyridinecarboxylate (i.e. the product of Example 8, Step D) (1.02 g, 3.09 mmol) in methanol (50 mL) was stirred at room temperature with an aqueous solution of sodium hydroxide (10 wt %, 2 mL) for 6 h. The reaction mixture was then concentrated and acidified with 1 N hydrochloric acid. The precipitated solids were isolated by filtration and dried to give the title acid as a white solid (0.9 g).

¹H NMR (DMSO-d₆) δ 8.28 (d, 1H), 8.00 (t, 1H), 7.82 (d, 2H), 6.60 (s, 1H), 4.40 (q, 2H), 1.45 (t, 3H), 1.31 (s, 9H).

### Step B: Preparation of 6-[[[3-(1,1-dimethylethyl)-1-ethyl-1H-pyrazol-5-yl]carbonyl]-amino]-2-pyridinecarboxamide

A procedure analogous to that of Example 6 was used to convert 6-[[[3-(1,1-dimethylethyl)-1-ethyl-1H-pyrazol-5-yl]carbonyl]amino-2-pyridine-carboxylic acid (520 mg) (i.e. the product of Step A) and dimethylamine (0.5 mL, 2.0 M in THF) to the title compound, a compound of present invention.

¹H NMR (CDCl₃) δ 8.46 (s, 1H, NH), 8.38 (d, 1H), 7.80 (t, 1H), 7.32 (dd, 1H), 6.55 (s, 1H), 4.60 (q, 2H), 3.14 (s, 3H), 3.02 (s, 3H), 1.43 (t, 3H), 1.30 (s, 9H).

### EXAMPLE 10

### Preparation of methyl 2-[[[3-(1,1-dimethylethyl)-1-ethyl-1H-pyrazol-5-yl]carbonyl]amino]-4-pyridinecarboxylate (Compound 151)

### Step A: Preparation of N-(4-methyl-2-pyridinyl)acetamide

A solution of 2-amino-4-picoline (25 g, 231 mmol) in acetic anhydride (150 mL) was heated to reflux for 10 h. The reaction mixture was then cooled to room temperature and concentrated to give a thick oily residue. The residue was dissolved in dichloromethane (400 mL) and washed sequentially with 1 N hydrochloric acid (50 mL) and water (50 mL). The organic phase was dried and concentrated to give the title compound as a white solid (30 g).

### Step B: Preparation of 4-(acetylamino)-2-pyridinecarboxylic acid

A procedure analogous to that of Example 8, Step B was used to convert N-(4-methyl-2-pyridinyl)acetamide (10 g) (i.e. the product of Step A) to the title acid, which was obtained as a solid (3.4 g).

### Step C: Preparation of methyl 4-amino-2-pyridinecarboxylate

A procedure analogous to that of Example 8, Step C was used to convert 4-(acetylamino)-2-pyridinecarboxylic acid (i.e. the product of Step B) (3.4 g) to the title compound (0.92 g).

¹H NMR (CDCl₃) δ 8.2 (d, 1H), 7.17 (d, 1H), 7.06 (s, 1H), 4.59 (br s, 2H, NH2), 3.92 (s, 3H).

### Step D: Preparation of methyl 2-[[[3-(1,1-dimethylethyl)-1-ethyl-1H-pyrazol-5-yl]-carbonyl]amino]-4-pyridinecarboxylate

A procedure analogous to that of Example 5, Step B was used to convert 3-(1,1-dimethylethyl)-1-ethyl-1H-pyrazole-5-carboxylic acid (i.e. the product of Example 1, Step D) (1.0 g) and methyl 4-amino-2-pyridinecarboxylate (i.e. the product of Step C) (0.78 g) to the title compound (0.85 g), a compound of present invention.

¹H NMR (CDCl₃) δ 8.60 (d, 1H), 7.92 (s, 1H, NH), 7.80 (d, 1H), 6.28 (s, 1H), 4.38 (q, 2H), 3.96 (s, 3H), 1.45 (t, 3H), 1.21 (s, 9H).

### EXAMPLE 11

### Preparation of 2-[[[3-(1,1-dimethylethyl)-1-ethyl-1H-pyrazol-5-yl]carbonyl]amino]-N,N-dimethyl-4-pyridinecarboxamide (Compound 156)

### Step A: Preparation of 2-[[[3-(1,1-dimethylethyl)-1-ethyl-1H-pyrazol-5-yl]carbonyl]-amino-4-pyridinecarboxylic acid

A procedure analogous to that of Example 9, Step A was used to convert methyl 2-[[[3-(1,1-dimethylethyl)-1-ethyl-1H-pyrazol-5-yl]carbonyl]amino]-4-pyridinecarboxylate (i.e. the compound of Example 10, Step D) (1.02 g, 3.09 mmol) to the title acid as a white solid (0.9 g).

¹H NMR (DMSO-d₆) δ 10.84 (s, 1H) 8.64 (s, 1H), 8.52 (d, 1H), 7.60 (d, 1H), 7/18 (s, 1H), 4.40 (q, 2H), 1.32 (t, 3H), 1.23 (s, 9H).

### Step B: Preparation of 2-[[[3-(1,1-dimethylethyl)-1-ethyl-1H-pyrazol-5-yl]carbonyl]-amino]-N,N-dimethyl-4-pyridinecarboxamide

A procedure analogous to that of Example 5, Step B was used to convert 2-[[[3-(1, 1-dimethylethyl)-1-ethyl-1H-pyrazol-5-yl]carbonyl]amino-4-pyridinecarboxylic acid (i.e. the compound of Step A) (200 mg) and dimethylamine to the title compound (110 mg), a compound of present invention.

¹H NMR (CDCl₃) δ 8.68 (s, 1H, NH), 8.40 (d, 2H), 7.04 (d, 1H), 6.61 (s, 1H), 4.58 (q, 2H), 3.18 (s, 3H), 3.00 (s, 3H), 1.42 (t, 3H), 1.3.1 (s, 9H).

### EXAMPLE 12

### Preparation of N-[3-[(diethylamino)carbonyl]phenyl]-1-(1,1-dimethylethyl)-4-ethyl-1H-pyrazole-3-carboxamide (Compound 168)

### Step A: Preparation of ethyl 1-(1,1-dimethylethyl)-3-ethyl-1H-pyrazole-4-carboxylate and ethyl 1-(1,1-dimethylethyl)-4-ethyl-1H-pyrazole-3-carboxylate

Ethyl 2-pentynoate (5.32 g, 42.2 mmol) was added to a solution of 3-(1,1-dimethylethyl)sydnone (6 g, 42.2 mmol) in xylenes (75 mL) under a nitrogen atmosphere. The reaction mixture was heated to reflux for three days and cooled to room temperature. The resulting white solids were removed by filtration using xylenes for rinsing. The filtrate was concentrated to leave a liquid, which was applied to a silica gel flash column (eluted with hexanes followed by 5:95 ethyl acetate-hexanes) to give the two title isomeric products as oils. Ethyl 1-(1,1-dimethylethyl)-3-ethyl-1H-pyrazole-4-carboxylate (3.62 g) was the major isomer. Ethyl 1-(1,1-dimethylethyl)-4-ethyl-1H-pyrazole-3-carboxylate (0.78 g) was the minor isomer.

¹H NMR (CDCl₃) δ major isomer: 7.92 (s, 1H), 4.2 (q, 2H), 2.88 (q, 2H), 1.57 (s, 9H), 1.3 (t, 3H), 1.2 (t, 3H); minor isomer: 7.34 (s, 1H), 4.4 (q, 2H), 2.7 (q, 2H), 1.6 (s, 9H), 1.39 (t, 3H), 1.20 (t, 3H).

### Step B: Preparation of 1-(1,1-dimethylethyl)-4-ethyl-1H-pyrazole-3-carboxylic acid

A solution of ethyl 1-(1,1-dimethylethyl)-4-ethyl-1H-pyrazole-3-carboxylate (i.e. minor isomer product of Step A) (0.75 g, 3.34 mmol) in ethanol (13 mL) was stirred at room temperature with aqueous sodium hydroxide (3 M, 6.7 mL, 20.0 mmol) for 2 days. The reaction mixture was then concentrated, and the pH of the resulting residue was adjusted to 2 with 1 N hydrochloric acid. The aqueous layer was extracted with diethyl ether (3×). The combined organic extracts were washed with brine solution, dried (Na₂SO₄) and concentrated to leave an oil (0.82 g). The oil solidified on standing, and the resulting solids were isolated using filtration and rinsed with hexanes to yield the title acid as a solid (0.53 g).

¹H NMR (CDCl₃) δ 7.38 (s, 1H), 2.77 (q, 2H), 1.6 (s, 9H), 1.21 (t, 3H).

### Step C: Preparation of N-[3-[(diethylamino)carbonyl]phenyl]-1-(1,1-dimethylethyl)-4-ethyl-1H-pyrazole-3-carboxamide

A solution of 1-(1,1-dimethylethyl)-4-ethyl-1H-pyrazole-3-carboxylic acid (i.e. product of Step B) (0.1 g, 0.51 mmol) in thionyl chloride (5 mL) was heated at reflux for about four hours. The reaction mixture was concentrated to yield the corresponding acid chloride as a liquid. The acid chloride was added to a solution of 3-amino-N,N-diethylbenzamide (0.117 g, 0.611 mmol) and triethylamine (107 μL, 0.764 mmol) in dichloromethane (2 mL). After stirring at room temperature overnight, the reaction mixture was concentrated and the resulting residue was partitioned between dichloromethane and 1 N hydrochloric acid. The dichloromethane layer was washed sequentially with 1 N hydrochloric acid and brine, dried and concentrated to give an oil. The oil was purified by column chromatography on silica gel to give the title compound (60 mg), a compound of present invention.

¹H NMR (CDCl₃) δ 8.86 (br s, 1H), 7.8 (d, 1H), 7.6 (s, 1H), 7.36 (t, 1H), 7.08 (d, 1H), 3.5 (m, 2H), 3.3 (m, 2H), 2.87 (q, 2H), 1.6 (s, 9H), 1.3 (m, 3H), 1.24 (t, 3H), 1.1 (m, 3H).

### EXAMPLE 13

### Preparation of N-[3-[(diethylamino)carbonyl]phenyl]-1-(1,1-dimethylethyl)-3-ethyl-1H-pyrazole-4-carboxamide (Compound 182)

### Step A: Preparation of 1-(1,1-dimethylethyl)-3-ethyl-1H-pyrazole-4-carboxylic acid

A procedure analogous to that of Example 12, Step B was used to convert ethyl 1-(1,1-dimethylethyl)-3-ethyl-1H-pyrazole-4-carboxylate (i.e. the major isomer product of Example 12, Step A) (1.76 g, 7.76 mmol) to the title acid (1.08 g).

¹H NMR (CDCl₃) δ 8.0 (s, 1H), 2.9 (q, 2H), 1.58 (s, 9H), 1.26 (t, 3H).

### Step B: Preparation of N-[3-[(diethylamino)carbonyl]phenyl]-1-(1,1-dimethylethyl)-3-ethyl-1H-pyrazole-4-carboxamide

A procedure analogous to that of Example 12, Step C was used to convert 1-(1,1-dimethylethyl)-3-ethyl-1H-pyrazole-4-carboxylic acid (i.e. the product of Step A) (100 mg) and 3-amino-N,N-diethylbenzamide (0.117 g, 0.611 mmol) to the title compound (91 mg), a compound of present invention.

¹H NMR (CDCl₃) δ 8.0 (s, 1H), 7.7 (br s, 1H), 7.6 (d, 1H), 7.5 (s, 1H), 7.3 (t, 1H), 7.0 (d, 1H), 3.5 (m, 2H), 3.3 (m, 2H), 2.9 (q, 2H), 1.6 (s, 9H), 1.32 (t, 3H), 1.3 (m, 3H), 1.1 (m, 3H).

### EXAMPLE 14

### Preparation of 4-bromo-1-(1,1-dimethylethyl)-N-[3-[(ethylamino)carbonyl]phenyl]-1H-pyrazole-3-carboxamide (Compound 221)

### Step A: Preparation of ethyl 1-(1,1-dimethylethyl)-1H-pyrazole-3-carboxylate

Ethyl propiolate (6.9 g, 70.3 mmol) was added to a solution of 3-(1,1-dimethylethyl)-sydnone (65 g, 35.2 mmol) in toluene (60 mL) under a nitrogen atmosphere. The reaction mixture was heated to reflux for two days and cooled to room temperature. The resulting white solid was removed by filtration using hexanes for rinsing. The filtrate was concentrated to leave a liquid, which was applied to a silica gel flash column (eluted with 100% hexanes followed by 10:90 ethyl acetate-hexanes) to give the title product (2.61 g) as a major isomer.

¹H NMR (CDCl₃) δ 7.5 (s, 1H), 6.7 (m, 1H), 4.4 (q, 2H), 1.63 (s, 9H), 1.39 (t, 3H).

### Step B: Preparation of ethyl 4-bromo-1-(1,1-dimethylethyl)-1H-pyrazole-3-carboxylate

To a solution of ethyl 1-(1,1-dimethylethyl)-1H-pyrazole-3-carboxylate (i.e. the product of Step A) (0.1 g, 0.509 mmol) in DMF (3.0 mL) at room temperature was added N-bromosuccinimide (0.90 mg, 0.509 mmol). After heating to 60° C. for 4 h, the reaction mixture was cooled to room temperature and partitioned between water and diethyl ether (3×50 mL). The organic extracts were washed with water (4×) and brine, dried (Na₂SO₄) and then concentrated to give the title product as an oil (0.126 mg).

¹H NMR (CDCl₃) δ 7.6 (s, 1H), 4.4 (q, 2H), 1.6 (s, 9H), 1.4 (t, 3H).

### Step C: Preparation of ethyl 4-bromo-1-(1,1-dimethylethyl)-1H-pyrazole-3-carboxylic acid

A procedure analogous to that of Example 12, Step B was used to hydrolyze ethyl 4-bromo-1-(1,1-dimethylethyl)-1H-pyrazole-3-carboxylate (i.e. the product of Step B) (0.61 g, 2.18 mmol) to give the title acid (0.4 g) as a solid.

¹H NMR (CDCl₃) δ 7.6 (s, 1H), 1.6 (s, 9H).

### Step D: Preparation of 4-bromo-1-(1,1-dimethylethyl)-N-[3-[(ethylamino)carbonyl]-phenyl]-1H-pyrazole-3-carboxamide

A procedure analogous to that of Example 12, Step C was used to convert ethyl 4-bromo-1-(1,1-dimethylethyl)-1H-pyrazole-3-carboxylic acid (i.e. product of Step C) (100 mg, 0.405 mmol) and 3-amino-N-ethylbenzamide (70 mg, 0.425 mmol) to the title compound (72 mg), a compound of present invention.

¹H NMR (CDCl₃) δ 8.82 (br s, 1H), 8.18 (s, 1H), 7.81 (d, 1H), 7.6 (s, 1H), 7.56 (d, 1H), 7.42 (t, 1H), 6.27 (br s, 1H), 3.5 (m, 2H), 1.6 (s, 9H), 1.27 (t, 3H).

### EXAMPLE 15

### Preparation of N-(2,3-dihydro-2-methyl-1-oxo-1H-isoindol-4-yl)-3-(1,1-dimethylethyl)-1-ethyl-1H-pyrazole-5-carboxamide (Compound 55)

### Step A: Preparation of 2,3-dihydro-2-methyl-4-nitro-1H-isoindol-1-one

To a solution of methyl 2-(bromomethyl))-3-nitrobenzoate (2.97 g, 10.8 mmol) prepared according to P. Japtap et al. (PCT Application Publication WO 01/77075 A2) in methanol (6 mL) was added a solution of methylamine in methanol (2.0 M, 20 mL). After stirring at room temperature for 3 h, the methanol was evaporated in vacuum and the residue was washed with ether and water to give the title compound as a white solid (0.98 g, 50% yield).

¹H NMR (CDCl₃): δ 8.39 (d, 1H), 8.18 (d, 1H), 7.70 (t, 1H), 7.27 (s, 1H), 4.87 (s, 2H), 3.27 (t, 3H).

### Step B: Preparation of 4-amino-2,3-dihydro-2-methyl-1H-isoindol-1-one

A slurry of 2,3-dihydro-2-methyl-4-nitro-1H-isoindol-1-one (i.e. product of Step A) (0.97 g, 5.1 mmol) and 10% palladium on carbon (0.24 g) in ethyl acetate (35 mL) was hydrogenated at 45 psi (310 kPa) at room temperature for 5.5 h. The mixture was then filtered through a pad of Celite® diatomaceous filter aid, and the Celite® was extracted with ethyl acetate. The filtrate was concentrated under vacuum to give the title compound (0.81 g, 97% yield).

¹H NMR (CDCl₃) δ 7.29 (m, 2H), 6.80 (d, 1H), 4.21 (s, 2H), 3.20 (s, 3H).

### Step C: Preparation of N-(2,3-dihydro-2-methyl-1-oxo-1H-isoindol-4-yl)-3-(1,1-dimethylethyl)-1-ethyl-1H-pyrazole-5-carboxamide

4-Amino-2,3-dihydro-2-methyl-1H-isoindol-1-one (i.e. the product of Step B) (0.15 g, 0.9 mmol) and triethylamine (0.187 g) was dissolved in dichloromethane (4 mL). 3-(1,1-Dimethylethyl)-1-ethyl-1H-pyrazole-5-carbonyl chloride (i.e. the product of Example 1, Step E) (0.318 g) was added to the reaction mixture, which was then stirred at room temperature for 2 days. Ethyl acetate (20 mL) and water (2 mL) were added, and the reaction mixture was passed through a Varian Chem Elut filter containing diatomaceous filter aid. The solvent was removed under vacuum, and the residue was triturated with 30% ethyl acetate in hexane to give the title compound, a compound of the invention, as a white solid (0.19 g, 55% yield).

¹H NMR (CDCl₃) δ 7.74 (d, 1H), 7.72 (s, 1H), 7.58 (d, 1H), 7.55 (t, 1H), 6.52 (s, 1H), 4.57 (q, 2H), 4.43 (s, 2H), 3.20 (s, 3H), 1.45 (t, 3H), 1.34 (s, 9H).

### EXAMPLE 16

### Preparation of N-[5-[(dimethylamino)carbonyl]-2-fluorophenyl]-1-(1,1-dimethylethyl)-3-ethyl-1H-pyrazole-4-carboxamide (Compound 222)

### Step A: Preparation of 4-fluoro-N,N-dimethyl-3-nitrobenzamide

4-Fluoro-3-nitrobenzoic acid (5 g, 27.0 mmol) was heated at reflux in thionyl chloride (20 mL) for 4 h. The reaction mixture was concentrated, diluted with dichloromethane and then reconcentrated to provide the acid chloride as a liquid. The acid chloride was then diluted with dichloromethane (50 mL). Half of the acid chloride solution was placed in a round-bottom flask and further diluted with dichloromethane to give a total volume of 50 mL. The acid chloride solution was cooled to 0° C. Triethylamine (3.0 g, 29.7 mmol) was added to the reaction mixture, and then a solution of 40% aqueous solution of dimethylamine (1.52 g, 13.5 mmol) in dichloromethane (20 mL) was added dropwise to the reaction mixture at such a rate that the temperature of the reaction mixture did not exceed 5° C. The cooled reaction mixture was stirred for 15 minutes more, and then hydrochloric acid (1 N) was added. The layers were separated, and the organic layer was washed with water, saturated aqueous sodium bicarbonate solution and brine, and then dried over sodium sulfate and concentrated to give the title compound (1.87 g).

¹H NMR (CDCl₃) δ 8.1 (m, 1H), 7.7-7.8 (m, 1H), 7.3-7.4 (m, 1H,), 3.1 (s, 3H), 3.0 (s, 3H).

¹⁹F NMR (CDCl₃) δ−115.5.

### Step B: Preparation of 3-amino-4-fluoro-N,N-dimethylbenzamide

4-Fluoro-N,N-dimethyl-3-nitrobenzamide (i.e. the product of Step A) (1.76 g, 8.29 mmol) was dissolved in acetic acid (22 mL). The reaction mixture was heated to 85° C., and then iron powder (1.39 g) was added in portions. After the addition was complete, the reaction mixture was stirred at 85° C. for an additional 20 minutes. The mixture was then cooled to room temperature and concentrated. Solids were removed by filtration through Celite® diatomaceous filter aid, using ethyl acetate and water for rinsing. The layers in the filtrate were separated. The organic layer was washed sequentially with water, aqueous saturated sodium bicarbonate solution and brine, and then dried over sodium sulfate and concentrated to give the title compound (1.5 g).

¹H NMR (CDCl₃): δ 6.9-7.0 (m, 1H), 6.8 (m, 1H), 6.69-7.78 (m, 1H), 3.8 (br s, 2H, NH₂), 3.0 (s, 3H), 2.9 (s, 3H).

### Step C: Preparation of N-[5-[(dimethylamino)carbonyl]-2-fluorophenyl]-1-(1,1-dimethylethyl)-3-ethyl-1H-pyrazole-4-carboxamide

A procedure analogous to that of Example 12, Step C was used to convert 1-(1,1-dimethylethyl)-3-ethyl-1H-pyrazole-4-carboxylic acid (i.e. the product of Example 13, Step A) (100 mg) and 3-amino-4-fluoro-N,N-dimethylbenzamide (92 mg, 0.509 mmol) (i.e. the product of Step B of Example 16) to the title compound (117 mg), a compound of present invention.

¹H NMR (CDCl₃) δ 8.5 (m, 1H), 7.9 (s, 1H), 7.7 (br s, 1H, NH), 7.1-7.2 (m, 2H), 3.1 (s, 3H), 3.0 (s, 3H), 2.9 (q, 2H), 1.6 (s, 9H), 1.3 (t, 3H).

¹⁹F NMR (CDCl₃) δ−130.2.

### EXAMPLE 17

### Preparation of 1-(1,1-dimethylethyl)-3-ethyl-N-[3-[(ethylamino)carbonyl]-4-fluorophenyl]-1H-pyrazole-4-carboxamide (Compound 241)

### Step A: Preparation of N-ethyl-2-fluoro-5-nitrobenzamide A procedure analogous to that of Example 16, Step A was used to convert 2-fluoro-5-nitrobenzoic acid (5 g, 27.0 mmol) and ethylamine (2 M in THF, 10 mL, 19.8 mmol) to give the title compound (1.6 g).

¹H NMR (CDCl₃) δ 8.9-9.0 (m, 1H), 8.3-8.4 (m, 1H), 7.2-7.3 (m, 1H), 6.6 (t, 1H), 3.5 (q, 2H), 1.2 (t, 3H).

¹⁹F NMR (CDCl₃) δ−105.2.

### Step B: Preparation of 5-amino-N-ethyl-2-fluorobenzamide

A procedure analogous to that of Example 16, Step B was used to convert N-ethyl-2-fluoro-5-nitrobenzamide (0.78 g, 3.68 mmol) and iron powder (0.62 g, 11.0 mmol) of acetic acid (10 mL) to give the title compound (0.62 g, oil).

¹H NMR (CDCl₃): δ 7.3 (m, 1H), 6.8-6.9 (m, 1H), 6.6-6.7 (m, 2H), 3.5 (q, 2H), 1.2 (t, 3H).

### Step C: Preparation of 1-(1,1-dimethylethyl)-3-ethyl-N-[3-[(ethylamino)carbonyl]-4-fluorophenyl]-1H-pyrazole-4-carboxamide

To a solution of 1-(1,1-dimethylethyl)-3-ethyl-1H-pyrazole-4-carboxylic acid (i.e. the product of Example 13, Step A) (150 mg, 0.76 mmol) in dichloromethane (4 mL) was added sequentially 1-propanephosphonic acid cyclic anhydride (50% in ethyl acetate, 365 mg, 1.14 mmol), 4-(dimethylamino)pyridine (140 mg, 1.14 mmol), 5-amino-N-ethyl-2-fluoro-benzamide (i.e. the product of Step B) (146 mg, 0.80 mmol) at room temperature. After stirring at room temperature overnight, the reaction mixture was diluted with 1 N hydrochloric acid (3.5 mL) and then filtered thru an Extube™ (tube containing diatomaceous earth marketed by Varian, Inc., 24201 Frampton Avenue, Harbor City, Calif. 90710 USA), which was rinsed well with dichloromethane. The filtrate was concentrated to leave the crude product as an oil. The crude product was purified by chromatography on silica gel to give an oil. Trituration with diethyl ethyl and hexanes provided the title product, a compound of the present invention, as a white solid, m.p. 168-169° C.

¹H NMR (CDCl₃) δ 8.2-8.3 (m, 1H), 7.9 (s, 1H), 7.8 (m, 1H), 7.7 (br s, 1H, NH), 7.1-7.2 (m, 1H), 6.7 (t, 1H), 3.5 (q, 2H), 2.9 (q, 2H), 1.59 (s, 9H), 1.3 (t, 3H), 1.2 (t, 3H).

¹⁹F NMR (CDCl₃) δ−120.2.

### EXAMPLE 18

### Preparation of N-[3-[(dimethylamino)carbonyl]-4-fluorophenyl]-3-(1,1-dimethylethyl)-1-ethyl-1H-pyrazole-5-carboxamide (Compound 236)

A procedure analogous to that of Example 17, Step C was used to convert 3-(1,1-dimethylethyl)-1-ethyl-1H-pyrazole-5-carboxylic acid (i.e. the product of Example 1, Step D) (200 mg, 1.0 mmol), 1-propanephosphonic acid cyclic anhydride (50% in ethyl acetate, 490 mg 1.5 mmol), 4-(dimethylamino)pyridine (187 mg, 1.5 mmol) and 5-amino-N,N-dimethyl-2-fluorobenzamide (195 mg, 1.0 mmol) in dichloromethane (4 mL) to the title product, a compound of the present invention, m.p. 93-95° C.

¹H NMR (CDCl₃) δ 8.7 (br s, 1H, NH), 7.7-7.8 (m, 1H), 7.4 (m, 1H), 6.9-7.0 (m, 1H), 6.6 (s, 1H), 4.5 (q, 2H), 3.1 (s, 3H), 2.9 (s, 3H), 1.42 (t, 3H), 1.4 (s, 9H).

¹⁹F NMR (CDCl₃) δ−121.0.

### EXAMPLE 19

### Preparation of ethyl 5-[[[3-(1,1-dimethylethyl)-1-ethyl-1H-pyrazol-5-yl]carbonyl]amino-3-pyridinecarboxylate (Compound 255)

### Step A: Preparation of 5-bromo-3-pyridinecarboxylic acid

Thionyl chloride (96.74 g, 58.9 mL, 0.813 mol) was added to 3-pyridinecarboxylic acid (also named nicotinic acid) (20 g, 0.163 mol) and heated at reflux (˜80° C.) for 3 h. The thionyl chloride was then distilled off under reduced pressure. The resulting acid chloride was cooled to 0 to −5° C., and bromine (13 mL, 0.163 mol) was added. The reaction mixture was heated at 155° C. for 8-10 h, then cooled to room temperature and quenched with ice-cold water (200 mL) added dropwise, causing a white solid to form. The solid was collected using filtration and dried to provide the title compound (31.5 g, 94% yield).

### Step B: Preparation of 5-amino-3-pyridinecarboxylic acid

To a mixture of 5-bromo-3-pyridinecarboxylic acid (i.e. the product of Step A) (25 g, 0.124 mol) in aqueous ammonia (67.32 mL) was added copper sulphate pentahydrate (8.41 g), and the reaction mixture heated in an autoclave at 120° C. for 16 h. Progress of the reaction was monitored by thin layer chromatography, using ninhydrin to visualize the product. The reaction mixture was washed with saturated solution of sodium sulfide to remove copper ions and was then acidified to a pH of about 4-5 using concentrated hydrochloric acid, causing a solid to separate as the acidified mixture cooled. The solid was collected using filtration and dried to provide the title compound (12.9 g, 74% yield).

### Step C: Preparation of methyl 5-amino-3-pyridinecarboxylate

Over 30 minutes hydrogen chloride gas was bubbled through dry methanol (60 mL) cooled to 0-5° C. Then 5-amino-3-pyridinecarboxylic acid (i.e. the product of Step B) (6.0 g, 43 mmol) was added, and the reaction mixture was heated at 75° C. for 3 h. The reaction mixture was concentrated, the residue was poured into cold water (30 mL), and the pH of the resulting mixture was increased to 4-5 by adding sodium bicarbonate. The mixture was then extracted with ethyl acetate, and the ethyl acetate extract was washed with water and brine, and then dried (Na₂SO₄) and concentrated. The residue was triturated with ethyl acetate-petroleum ether to yield the title compound (4.2 g, 63% yield).

¹H NMR (CDCl₃) δ 8.63 (s, 1H, ArH), 8.25 (s, 1H, ArH), 7.57 (s, 1H, ArH), 3.93 (s, 3H, CH₃), 3.87 (br s, 2H, NH₂).

### Step D: Preparation of ethyl 5-[[[3-(1,1-dimethylethyl)-1-ethyl-1H-pyrazol-5-yl]carbonyl]amino-3-pyridinecarboxylate

To a solution of 3-(1,1-dimethylethyl)-1-ethyl-1H-pyrazole-5-carboxylic acid (i.e. the product of Example 1, Step D) (4.00 g, 2.84 mmol) in dry dichloromethane (35 mL) was added oxalyl chloride (3.88 g, 2.47 mL, 30.6 mmol) followed by a few drops of N,N-dimethylformamide. The resulting solution was stirred and heated to 45° C. for 2.5 h. The dichloromethane solvent and excess oxalyl chloride were removed by distillation under reduced pressure. The resulting residue was diluted with dichloromethane (20 mL) and added to a mixture of methyl 5-amino-3-pyridinecarboxylate (i.e. the product of Step C) (2.98 g, 24.4 mmol) and triethylamine (4.12 g, 5.67 mL, 42.8 mmol) in dichloromethane (20 mL) at 0° C. The reaction mixture was gradually warmed to room temperature and then heated at 45° C. for 12 h. The dichloromethane solvent was removed by distillation under reduced pressure, and the residue was quenched with ice water and extracted with dichloromethane (3×30 mL). The combined organic extracts were then washed with water and brine. The solution was dried over sodium sulfate and filtered, and the solvent was removed to give the crude product. The crude product was purified by column chromatography (60-120 mesh silica gel, 20% ethyl acetate-petroleum ether) to provide the title product (5.1 g, 78% yield), a compound of the present invention.

¹H NMR (CDCl₃) δ 9.0 (m, 2H, ArH), 8.71 (s, 1H, ArH), 8.01 (s, 1H, ArH), 6.58 (s, 1H, ArH), 4.58 (q, J=7.2 Hz, 2H, CH₂), 3.98 (s, 3H, CH₃), 1.46 (t, J=7.2 Hz, 3H, CH₃), 1.34 (s, 9H, 3 CH₃).

### EXAMPLE 20

### Preparation of 5-[[[3-(1,1-dimethylethyl)-1-ethyl-1H-pyrazol-5-yl]carbonyl]amino]-N,N-diethyl-3-pyridinecarboxamide (Compound 261)

### Step A: Preparation of 5-[[[3-(1,1-dimethylethyl)-1-ethyl-1H-pyrazol-5-yl]carbonyl]amino]-3-pyridinecarboxylic acid

To a solution of ethyl 5-[[[3-(1,1-dimethylethyl)-1-ethyl-1H-pyrazol-5-yl]carbonyl]-amino-3-pyridinecarboxylate (i.e. the product of Example 19, Step D) (3.11 g, 9.77 mmol) in tetrahydrofuran (20 mL) was added a solution of lithium hydroxide (0.938 g, 39 mmol) in water (10 mL). The reaction mixture was stirred at room temperature for 24 h. The solvent was then evaporated under reduced pressure, and the residue was diluted with water, acidified with hydrochloric acid (1.5 N) to a pH of about 4-5 and extracted with ethyl acetate (2×15 mL). The combined organic extracts were washed with cold water and brine, and then dried (Na₂SO₄). The solvent was removed by evaporation to leave the title compound (2.4 g, 92% yield).

¹H NMR (DMSO-d₆) δ 13.50 (br s, 1H, OH), 10.49 (s, 1H, NH), 9.10 (s, 1H, ArH), 8.80 (s, 1H, ArH), 8.71 (s, 1H, ArH), 7.00 (s, 1H, ArH), 4.46 (q, J=6.78 Hz, 2H, CH₂), 1.15-1.35 (m, 12H, 4 CH₃).

### Step B: Preparation of 5-[[[3-(1,1-dimethylethyl)-1-ethyl-1H-pyrazol-5-yl]carbonyl]amino]-N,N-diethyl-3-pyridinecarboxamide

To a solution of 5-[[[3-(1,1-dimethylethyl)-1-ethyl-1H-pyrazol-5-yl]carbonyl]amino]-3-pyridinecarboxylic acid (i.e. the product of Step A) (250 mg, 0.793 mmol) in dichloromethane (5 mL) at room temperature under nitrogen atmosphere was added sequentially 1-propanephosphonic acid cyclic anhydride (50% in ethyl acetate, 2 mL, 3.4 mmol), diethylamine (0.5 mL, 5 mmol) and 4-(dimethylamino)pyridine (0.1 g, 0.8 mmol). The reaction mixture was stirred at room temperature for 6 h and then diluted with additional dichloromethane (10 mL) and washed with hydrochloric acid (1 N, 5 mL). The organic phase was separated, dried and concentrated, and the residue was purified using flash chromatography to provide the title product, a compound of the present invention, as a solid (256 mg, 84% yield).

¹H NMR (CDCl₃) δ 8.70 (s, 1H,), 8.42 (s, 1H), 8.18 (m, 1H,), 6.58 (s, 1H), 4.57 (q, 2H), 3.60 (m, 4H), 1.46 (t, 3H), 1.34 (s, 9H), 1.26 (m, 6H).

### EXAMPLE 21

### Preparation of N-[5-[(dimethylamino)carbonyl]-2-fluorophenyl]-3-(1,1-dimethylethyl)-1-ethyl-4-fluoro-1H-pyrazole-5-carboxamide (Compound 276)

A solution of N-[5-[(dimethylamino)carbonyl]-2-fluorophenyl]-3-(1,1-dimethylethyl)-1-ethyl-1H-pyrazole-5-carboxamide (i.e. the product of Example 7, Step D) (0.32 g, 0.88 mmol) and SELECTFLUOR™ fluorinating reagent (1-(chloromethyl-4-fluoro-1,4-diazonia-bicyclo[2.2.2]octane bis(tetrafluoroborate)) (0.72 g, 1.97 mmol) in acetonitrile (10 mL) was heated to reflux for 5 h. The mixture was cooled to room temperature and concentrated, and the residue was diluted with equal volumes of water and dichloromethane. The organic layer was separated and concentrated. The residue was purified by flash column chromatography on silica gel to provide the title product, a compound of the present invention, as a white solid (0.14 g, 42% yield).

¹H NMR (CDCl₃) δ 8.5 (d, 1H), 8.3 (d, 1H), 7.2 (br s, 1H), 7.17 (m, 1H), 4.55 (q, 2H), 3.1 (d, 6H), 1.43 (t, 3H), 1.37 (s, 9H).

### EXAMPLE 22

### Preparation of ethyl 3-[[[3-(1,1-dimethylethyl)-1-ethyl-4,5-dihydro-1H-pyrazol-5-yl]carbonyl]amino]-4-fluorobenzoate (Compound 267)

### Step A: Preparation of 3,3-dimethyl-1-(triphenylphosphoranylidene)-2-butanone

To a solution of triphenylphosphine (10.74 g, 40.9 mmol) in chloroform (25 mL) was added dropwise 1-bromo-3,3-dimethyl-2-butanone (7.33 g, 40.9 mmol). The cloudy solution was stirred at room temperature overnight. The solvent was removed in vacuo to give a white solid, which was then stirred overnight with saturated aqueous sodium bicarbonate (200 mL) at room temperature. The white solid was then collected by filtration and dried in a vacuum oven to a constant weight of the title compound (13.7 g).

¹H NMR (CDCl₃): δ 7.8-7.3 (m, 15H), 3.80 (d, 1H), 1.20 (s, 9H).

### Step B: Preparation of butyl (2E)-5,5-dimethyl-4-oxo-2-hexenoate

A slurry of the 3,3-dimethyl-1-(triphenylphosphoranylidene)-2-butanone (i.e. the product of Step A) (12.5 g, 34.5 mmol) and butyl oxoacetate (4.5 g, 34.5 mmol) in toluene (200 mL) was stirred for 3 days at room temperature. The toluene solvent was removed in vacuo to leave an orange solid as crude product, which was then purified by column chromatography (10% ethyl acetate in hexane) to provide the title compound (5 g) as the trans isomer.

¹H NMR (CDCl₃) δ 7.51 (d, 1H), 6.77 (d, 1H), 4.21 (t, 2H), 1.68 (m, 2H), 1.41 (m, 2H), 1.20 (s, 9H), 0.95 (t, 3H).

### Step C: Preparation of butyl 3-(1,1-dimethylethyl)-1-ethyl-4,5-dihydro-1H-pyrazole-5-carboxylate

A slurry of butyl (2E)-5,5-dimethyl-4-oxo-2-hexenoate (i.e. the product of Step B) (6.5 g, 30.9 mmol), ethylhydrazine ethanedioate (1:1) (5.6 g, 37.1 mmol), and N,N-diisopropylethylamine (5.2 g, 40.2 mmol) in methanol (65 mL) was stirred for 5 days at room temperature. The solvent was removed in vacuo, and the residue was purified by column chromatography (3-13% ethyl acetate in hexane) to give the title compound (4.8 g).

¹H NMR (CDCl₃) δ 4.18 (m, 2H), 3.62 (dd, 1H), 3.06 (m, 2H), 2.95 (m, 2H), 1.62 (m, 2H), 1.39 (m, 2H), 1.18 (t, 3H), 1.15 (s, 9H), 0.94 (t, 3H).

### Step D: Preparation of 3-(1,1-dimethylethyl)-1-ethyl-4,5-dihydro-1H-pyrazole-5-carboxylic acid

Butyl 3-(1,1-dimethylethyl)-1-ethyl-4,5-dihydro-1H-pyrazole-5-carboxylate (i.e. the product of Step C) (1.8 g, 7.1 mmol) was dissolved in ethanol (20 mL), and aqueous sodium hydroxide (10%, 5.7 g) was added. The solution was stirred overnight at room temperature. Most of the ethanol solvent was removed in vacuo, and then the pH of the residual solution was adjusted to 2 using hydrochloric acid (1 N). The cloudy mixture was extracted with ethyl acetate (2×). The combined organic extracts were dried (MgSO₄), and the solvent was removed in vacuo to provide the title compound (0.64 g).

¹H NMR (CDCl₃) δ 3.75 (dd, 1H), 3.24 (m, 1H), 3.05 (m, 1H), 2.92 (dd, 1H), 1.18 (t, 3H), 1.16 (s, 9H).

### Step E: Preparation of ethyl 3-[[[3-(1,1-dimethylethyl)-1-ethyl-4,5-dihydro-1H-pyrazol-5-yl]carbonyl]amino]-4-fluorobenzoate

To a stirred solution of 3-(1,1-dimethylethyl)-1-ethyl-4,5-dihydro-1H-pyrazole-5-carboxylic acid (i.e. the product of Step D) (0.8 g, 4.1 mmol) in dichloromethane (5 mL) was added 1-propanephosphonic acid cyclic anhydride (50 wt % solution in ethyl acetate, 3.9 g, 6.14 mmol) followed by 4-(dimethylamino)pyridine (0.75 g, 6.14 mmol). After stirring for 1 h, ethyl 3-amino-4-fluorobenzoate (0.68 g, 3.7 mmol) was added, and the resulting solution was stirred at room temperature overnight. The solvent was removed in vacuo and partitioned between water (50 mL) and ethyl acetate (100 mL). The aqueous layer was extracted with ethyl acetate (30 mL). The organic layer was washed with aqueous saturated sodium bicarbonate (50 mL) and water (50 mL), and dried (MgSO₄). The solvent was removed in vacuo to provide the title product, a compound of the present invention, as an oil (1.36 g).

¹H NMR (CDCl₃) δ 9.3 (s, 1H), 8.95 (d, 1H), 7.80 (m, 1H), 7.16 (t, 1H), 3.70 (t, 1H), 3.33 (dd, 1H), 3.18 (dq, 1H), 2.95 (dq, 1H), 2.83 (dd, 1H), 1.39 (t, 3H), 1.21 (t, 3H), 1.17 (s, 9H).

### EXAMPLE 23

### Preparation of N-[5-[(dimethylamino)carbonyl]-2-fluorophenyl]-3-(1,1-dimethylethyl)-1-ethyl-4,5-dihydro-1H-pyrazole-5-carboxamide (Compound 268)

To a solution of ethyl 3-[[[3-(1,1-dimethylethyl)-1-ethyl-4,5-dihydro-1H-pyrazol-5-yl]carbonyl]amino]-4-fluorobenzoate (i.e. the product of Example 22) (1.0 g, 2.7 mmol) in ethanol (10 mL) was added aqueous sodium hydroxide (10%, 2.2 g). The solution was stirred overnight at room temperature and then concentrated in vacuo. The pH of the solution was adjusted to 2 using hydrochloric acid (1 N). Most of the water was removed in vacuo, and then the cloudy solution was extracted with ethyl acetate. The solvent was removed in vacuo from the organic extract to provide the acid in crude form (0.64 g), which was then dissolved in dichloromethane (20 mL), and oxalyl chloride (0.31 g) and N,N-dimethylformamide (one drop) were added. The resulting solution was stirred at room temperature overnight. The solvent was removed in vacuo, and more dichloromethane was added, and the solvent was again removed in vacuo. This process was repeated once more to provide the acid chloride in crude form (0.61 g). The acid chloride (0.3 g) was combined with a tetrahydrofuran solution of dimethylamine (2 M, 5 mL), and the reaction mixture was stirred overnight at room temperature. The solvent was removed in vacuo, and the residue was diluted with ethyl acetate and washed with water. The ethyl acetate solution was dried (MgSO₄) and evaporated to leave the title product, a compound of the present invention, as an oil (0.20 g).

¹H NMR (CDCl₃) δ 9.3 (s, 1H), 8.42 (d, 1H), 7.15 (m, 2H), 4.12 (m, 1H), 3.70 (t, 1H), 3.30 (dd, 1H), 3.15 (m, 1H), 3.02 (br d, 6H), 2.95 (m, 1H), 2.80 (dd, 1H), 1.34-1.20 (m, 3H), 1.17 (s, 9H).

### EXAMPLE 24

### Preparation of 1-(1,1-dimethylethyl)-3-ethyl-N-[5-[(ethylamino)carbonyl]-2-fluorophenyl]-4,5-dihydro-1H-pyrazole-4-carboxamide (Compound 336)

### Step A: Preparation of 3-[(1,3-dioxopentyl)amino]-N-ethyl-4-fluorobenzamide

A solution of 3-amino-N-ethyl-4-fluorobenzamide (0.50 g, 1.8 mmol) and methyl 3-oxopentanoate (alternatively named methyl propionylacetate; 1.50 g, 11.5 mmol) was heated at 73-80° C. for 60 h. Upon cooling to room temperature, an off-white solid precipitated out; this was collected by filtration and washed successively with hexane and diethyl ether. The solid was dried under vacuum to give the title compound (0.42 g).

¹H NMR (CDCl₃) δ 9.65 (br s, 1H), 8.64 (dd, 1H), 7.63 (m, 1H), 7.17 (dd, 1H), 6.15 (br s, 1H), 3.61 (s, 2H), 3.48 (q, 2H), 2.62 (q, 2H), 1.24 (t, 3H), 1.14 (t, 3H).

### Step B: Preparation of 1-(1,1-dimethylethyl)-3-ethyl-N-[5-[(ethylamino)carbonyl]-2-fluorophenyl]-4,5-dihydro-1H-pyrazole-4-carboxamide

A solution of 3-[(1,3-dioxopentyl)amino]-N-ethyl-4-fluorobenzamide (i.e. the product of Step A) (0.36 g, 1.3 mmol) in methanol (2 mL) was added dropwise to a slurry of sodium acetate (0.165 g) and aqueous formaldehyde (37%, 0.145 g) over 2 minutes. The resulting yellowish solution was stirred at room temperature for 3 h and then partitioned between ethyl acetate (40 mL) and water (10 mL). The organic layer was washed with water (10 mL) and dried (MgSO₄), and the solvent was removed in vacuo to leave a gummy solid. This was then stirred with a mixture of tert-butylhydrazine hydrochloride (0.177 g) and anhydrous sodium carbonate (0.148 g) in methanol (3 mL) at room temperature for 20 h. Then the solvent was removed using a rotary evaporator, and the residue was purified by column chromatography (ethyl acetate—hexane) to provide 0.17 g of the title compound, a compound of the present invention, as a solid (0.17 g).

¹H NMR (CDCl₃) δ 9.42 (br s, 1H), 8.63 (dd, 1H), 7.61 (m, 1H), 7.13 (dd, 1H), 6.25 (br s, 1H), 3.64 (dd, 1H), 3.54 (dd, 1H), 3.46 (m, 2H), 3.25 (t, 1H), 2.41 (m, 2H), 1.23 (s, 9H), 1.23 (t, 3H), 1.17 (t, 3H).

### EXAMPLE 25

### Preparation of N-[5-[(dimethylamino)carbonyl]-2-fluorophenyl]-2-ethyl-2,4,5,6-tetrahydro-6,6-dimethyl-3-cyclopentapyrazolecarboxamide (Compound 352)

### Step A: Preparation of ethyl 2-hydroxy-3,3-dimethyl-α-oxo-1-cyclopentene-1-acetate 2,2-Dimethylcyclopentanone (9.6 g) was added to a mixture of diethyl oxalate (11.6 mL), 21% solution of sodium ethoxide in ethanol (11.6 mL) and ethanol (20 mL). The mixture was stirred at room temperature for 18 h. The mixture was then poured onto ice-cold water (200 mL) and acidified to pH 4-5 using acetic acid and extracted with diethyl ether (3×50 mL). The organic extracts were washed with water (3×50 mL) and dried (MgSO₄) and concentrated to provide the title compound as an oil (17.58 g).

¹H NMR (CDCl₃) δ 12.9 (br s, 1H), 4.36 (q, 2H), 2.9 (t, 2H), 1.83 (t, 2H), 1.4 (t, 3H), 1.13 (s, 6H).

### Step B: Preparation of tautomeric mixture of ethyl 2,4,5,6-tetrahydro-6,6-dimethyl-3-cyclopyrazolecarboxylate and ethyl 1,4,5,6-tetrahydro-6,6-dimethyl-3-cyclopentapyrazolecarboxylate

Hydrazine hydrate (2.5 mL) was added dropwise to ethyl 2-hydroxy-3,3-dimethyl-α-oxo-1-cyclopentene-1-acetate (i.e. the product of Step A) (10 g) dissolved in acetic acid (25 mL) at room temperature, and the mixture was stirred for a further 2 h. The reaction mixture was poured onto ice water (200 mL) and extracted with ethyl acetate (4×50 mL), dried (MgSO₄) and concentrated to provide a yellow solid residue. The residue was chromatographed on silica gel using 6:4 hexanes-ethyl acetate as eluant to provide the title tautomeric mixture as an orange solid (7.8 g)

¹H NMR (CDCl₃) δ 4.36 (q, 2H), 2.8 (t, 2H), 2.29 (t, 2H), 1.4-1.2 (m, 9H).

### Step C: Preparation of ethyl 2-ethyl-2,4,5,6-tetrahydro-6,6-dimethyl-3-cyclopentapyrazolecarboxylate and ethyl 1-ethyl-1,4,5,6-tetrahydro-6,6-dimethyl-3-cyclopentapyrazolecarboxylate

To a solution of a tautomeric mixture of ethyl 2,4,5,6-tetrahydro-6,6-dimethyl-3-cyclopyrazolecarboxylate and ethyl 1,4,5,6-tetrahydro-6,6-dimethyl-3-cyclopentapyrazole-carboxylate (i.e. the product of Step B) (7.69 g) in N,N-dimethylfommamide (50 mL), potassium carbonate (7.71 g) and tetrabutylammonium bromide (100 mg) were added. Ethyl iodide (4.44 mL) was added at once, and the mixture was stirred at room temperature for 18 h. The mixture was poured into water (200 mL) and extracted with diethyl ether (3×100 mL). The organic phase was washed with water (3×50 mL) and dried (MgSO₄) and concentrated to provide residue containing mixture of ethyl 2-ethyl-2,4,5,6-tetrahydro-6,6-dimethyl-3-cyclopentapyrazolecarboxylate and ethyl 1-ethyl-1,4,5,6-tetrahydro-6,6-dimethyl-3-cyclopentapyrazolecarboxylate. The residue was chromatographed on silica gel using as eluant hexanes-ethyl acetate (9:1, 8:2, 7:3 and 1:1); the earlier fractions contained ethyl 2-ethyl-2,4,5,6-tetrahydro-6,6-dimethyl-3-cyclopentapyrazolecarboxylate. The fractions were combined and concentrated to provide ethyl 2-ethyl-2,4,5,6-tetrahydro-6,6-dimethyl-3-cyclopentapyrazolecarboxylate (3.7 g). The later fractions contained ethyl 1-ethyl-1,4,5,6-tetrahydro-6,6-dimethyl-3-cyclopentapyrazolecarboxylate. These fractions were combined and concentrated to provide 1-ethyl-1,4,5,6-tetrahydro-6,6-dimethyl-3-cyclopentapyrazolecarboxylate (3.5 g). Ethyl 2-ethyl-2,4,5,6-tetrahydro-6,6-dimethyl-3-cyclopentapyrazolecarboxylate:

¹H NMR (CDCl₃): δ 4.53 (q, 2H), 4.31 (q, 2H), 2.75 (t, 2H), 2.21 (t, 2H), 1.42-1.3 (m, 12H).

Ethyl 1-ethyl-1,4,5,6-tetrahydro-6,6-dimethyl-3-cyclopentapyrazolecarboxylate:

¹H NMR (CDCl₃) δ 4.37 (q, 2H), 4.13 (q, 2H), 2.73 (t, 2H), 2.36 (t, 2H), 1.49 (t, 3H), 1.39 (m, 9H).

### Step D: Preparation of 2-ethyl-2,4,5,6-tetrahydro-6,6-dimethyl-3-cyclopentapyrazolecarboxylic acid

To a solution of ethyl 2-ethyl-2,4,5,6-tetrahydro-6,6-dimethyl-3-cyclopentapyrazole-carboxylate (i.e. first eluted product of Step C) (3.63 g) in tetrahydrofuran (25 mL), aqueous sodium hydroxide (1 N, 23.1 mL) was added, and the mixture was stirred at room temperature for 18 h. Then the mixture was acidified with hydrochloric acid (6 N) and extracted with dichloromethane (3×25 mL), dried (MgSO₄) and concentrated to provide the title compound as a white solid (3.1 g).

¹H NMR (CDCl₃) δ 4.56 (q, 2H), 2.84 (m, 2H), 2.24 (m, 2H), 1.42 (t, 3H), 1.32 (s, 6H).

### Step E: Preparation of N-[5-[(dimethylamino)carbonyl]-2-fluorophenyl]-2-ethyl-2,4,5,6-tetrahydro-6,6-dimethyl-3-cyclopentapyrazolecarboxamide

2-Ethyl-2,4,5,6-tetrahydro-6,6-dimethyl-3-cyclopentapyrazolecarboxylic acid (i.e. the product of Step D) (0.6 g) was dissolved in dichloromethane (2 mL), and one drop of N,N-dimethylformamide was added, followed by oxalyl chloride (0.25 mL), and the mixture was stirred at room temperature for 1 h and concentrated. The residue was dissolved in dichloromethane (2 mL) and then added to solution of 3-amino-4-fluoro-N,N-dimethyl-benzamide (i.e. the product of Example 7, Step C) (0.6 g) and triethylamine (0.5 mL). The mixture was stirred at room temperature for 2 h and then chromatographed on a column containing silica gel (10 g), using dichloromethane as eluant to provide the title product, a compound of the present invention, as a white solid (0.5 g).

¹H NMR (CDCl₃) δ 8.6 (d, 1H), 7.8 (br s, 1H), 7.19 (d, 2H), 4.6 (q, 2H), 3.1 (d, 6H), 2.95 (t, 2H), 2.4 (t, 2H), 1.44 (t, 3H), 1.26 (s, 6H).

### EXAMPLE 26

### Preparation of ethyl 3-[[[5-ethyl-2-(1-methylethyl)-2H-1,2,3-triazol-4-yl]carbonyl]amino]benzoate (Compound 367)

### Step A: Preparation of ethyl 5-ethyl-1,2,3-triazole-4-carboxylate

Ethyl 2-pentynoate (16.6 g, 0.132 mol) and trimethylsilylazide (38.0 g, 0.333 mol) were stirred at 100-110° C. under nitrogen for 70 h. After cooling and dilution with methanol (60 mL) a white solid precipitated. After evaporation of the mixture under reduced pressure, the residue was crystallized from ethyl ether to afford the title product as a white solid (15.7 g, 0.093 mol, 70% yield).

¹H NMR (CDCl₃) δ 4.42 (q, 2H), 3.07 (q, 2H), 1.37 (t, 3H), 1.32 (t, 3H).

### Step B: Preparation of ethyl 5-ethyl-2-(1-methylethyl)-2H-1,2,3-triazole-4-carboxylate and ethyl 4-ethyl-1-(1-methylethyl)-1H-1,2,3-triazole-5-carboxylate

A mixture of ethyl 5-ethyl-1,2,3-triazole-4-carboxylate (i.e. the product of Step A) (3.84 g, 22.7 mmol), potassium carbonate (5.64 g, 40.9 mmol) and 2-iodopropane (6.95 g, 40.9 mmol) in acetonitrile (68 mL) was stirred at 50-60° C. under nitrogen for 2 h. After cooling to room temperature, the mixture was filtered through a short pad of silica gel and rinsed with ethyl acetate. The solution was concentrated and the residue was purified by column chromatography to provide ethyl 5-ethyl-2-(1-methylethyl)-2H-1,2,3-triazole-4-carboxylate (2.87 g, 13.6 mmol, 60% yield), followed by its isomer ethyl 4-ethyl-1-(1-methylethyl)-1H-1,2,3-triazole-5-carboxylate (0.96 g, 4.54 mmol, 20% yield) as white solids.

Ethyl 5-ethyl-2-(1-methylethyl)-2H-1,2,3-triazole-4-carboxylate:

¹H NMR (CDCl₃) δ 4.82 (m, 1H), 4.42 (q, 2H), 2.95 (q, 2H), 1.58 (d, 6H), 1.41 (t, 3H), 1.28 (t, 3H).

Ethyl 4-ethyl-1-(1-methylethyl)-1H-1,2,3-triazole-5-carboxylate:

¹H NMR (CDCl₃) δ 5.42 (m, 1H), 4.42 (q, 2H), 2.94 (q, 2H), 1.58 (d, 6H), 1.39 (t, 3H), 1.28 (t, 3H).

### Step C: Preparation of ethyl 3-[[[5-ethyl-2-(1-methylethyl)-2H-1,2,3-triazol-4-yl]carbonyl]amino]benzoate

To a stirred solution of ethyl 5-ethyl-2-(1-methylethyl)-2H-1,2,3-triazole-4-carboxylate (i.e. the first eluted product of Step B) (1.119 g, 5.64 mmol) in tetrahydrofuran (15 mL) was added a solution of lithium hydroxide (0.54 g, 22.56 mmol) in water (15 mL). The mixture was stirred at room temperature overnight, and then partitioned between ether and water. The aqueous layer was acidified with hydrochloric acid (6 N) to pH 1-2 and extracted with ethyl acetate, dried (Na₂SO₄) and concentrated to provide the carboxylic acid intermediate as a white solid (0.94 g, 5.08 mmol, 90% yield). To a stirred solution of the carboxylic acid intermediate (0.78 g, 4.22 mmol) in dichloromethane (25 mL) was added oxalyl chloride (1.61 g, 12.7 mmol) dropwise at room temperature. After stirring the reaction mixture for 10 minutes, N,N-dimethylformamide (two drops) was added. The mixture was stirred for an additional 1.5 h and then concentrated to provide the acid chloride intermediate as a pale yellow oil. To a stirred solution of ethyl 3-aminobenzoate (0.70 g, 4.22 mmol), N,N-diisopropylethylamine (1.09 g, 8.44 mmol) in dichloromethane (15 mL) was added a solution of the acid chloride intermediate in dichloromethane (5 mL). The reaction mixture was stirred at room temperature for 2 h and then concentrated. The residue was chromatographed to afford the title product, a compound of the present invention, as a white solid (1.36 g, 4.10 mmol, 97% yield).

¹H NMR (CDCl₃) δ 8.62 (br s, 1H), 8.14 (d, 1H), 8.10 (s, 1H), 7.81 (d, 1H), 7.43 (t, 1H), 4.80 (m, 1H), 4.40 (q, 2H), 3.04 (q, 2H), 1.61 (d, 6H), 1.41 (t, 3H), 1.32 (t, 3H).

### EXAMPLE 27

### Preparation of N-[3-[(dimethylamino)carbonyl)phenyl]-5-ethyl-2-(1-methylethyl)-2H-1,2,3-triazole-4-carboxamide (Compound 358)

To a stirred solution of ethyl 3-[[[5-ethyl-2-(1-methylethyl)-2H-1,2,3-triazol-4-yl]carbonyl]amino]benzoate (i.e. the product of Example 26, Step C) (1.34 g, 4.04 mmol) in tetrahydrofuran (15 mL) was added a solution of lithium hydroxide (0.48 g, 20.2 mmol) in water (15 mL). The mixture was stirred at room temperature overnight, then partitioned between ether and water. The aqueous layer was acidified with hydrochloric acid (6 N) to pH 1-2 and extracted with ethyl acetate, dried (Na₂SO₄) and concentrated to provide the carboxylic acid intermediate as a white solid (1.10 g, 3.62 mmol, 90% yield). A mixture of the carboxylic acid intermediate (130 mg, 0.43 mmol), 4-(dimethylamino)pyridine (78 mg, 0.64 mmol), 1-propanephosphonic acid cyclic anhydride (50 wt % in EtOAc, 423 mg, 0.66 mmol), and dimethylamine (2.0 M in THF, 0.66 mL, 1.32 mmol) in dichloromethane (3 mL) was stirred at room temperature overnight. The mixture was concentrated and the residue was purified by column chromatography to afford the title product, a compound of the present invention, as a white solid (130 mg, 0.40 mmol, 92% yield).

¹H NMR (CDCl₃) δ 8.62 (br s, 1H), 7.77 (s, 1H), 7.71 (d, 1H), 7.39 (t, 1H), 7.18 (d, 1H), 4.80 (m, 1H), 2.98-3.10 (m, 8H), 1.60 (d, 6H), 1.32 (t, 3H).

### EXAMPLE 28

### Preparation of ethyl 3-[[[2-(1,1-dimethylethyl)-5-ethyl-2H-1,2,3-triazol-4-yl]carbonyl]amino]benzoate (Compound 360)

### Step A: Preparation of ethyl 2-(1,1-dimethylethyl)-5-ethyl-2H-1,2,3-triazole-4-carboxylate and ethyl 1-(1,1-dimethylethyl)-4-ethyl-1H-1,2,3-triazole-5-carboxylate

To a stirred solution of 5-ethyl-1,2,3-triazole-4-carboxylic acid ethyl ester (i.e. product of Step A of Example 26) (1.05 g, 6.25 mmol) and tert-butyl alcohol (0.93 g, 12.5 mmol) in trifluoroacetic acid (6 mL) was added concentrated sulfuric acid (0.61 g, 6.25 mmol). After stirring at room temperature for 14 h, the reaction mixture was partitioned between ethyl acetate and water. The organic layer was washed with water, saturated aqueous sodium carbonate and brine, and then dried (Na₂SO₄). After concentration, the residue was purified by column chromatography to afford ethyl 2-(1,1-dimethylethyl)-5-ethyl-2H-1,2,3-triazole-4-carboxylate (0.74 g, 3.76 mmol, 64% yield), followed by its isomer ethyl 1-(1,1-dimethyl-ethyl)-4-ethyl-1H-1,2,3-triazole-5-carboxylate (0.24 g, 1.22 mmol, 21% yield) as colorless oils.

Ethyl 2-(1,1-dimethylethyl)-5-ethyl-2H-1,2,3-triazole-4-carboxylate:

¹H NMR (CDCl₃) δ 4.41 (q, 2H), 2.93 (q, 2H), 1.68 (d, 9H), 1.40 (t, 3H), 1.27 (t, 3H).

Ethyl 1-(1,1-dimethylethyl)-4-ethyl-1H-1,2,3-triazole-5-carboxylate:

¹H NMR (CDCl₃) δ 4.40 (q, 2H), 2.87 (q, 2H), 1.77 (d, 6H), 1.42 (t, 3H), 1.29 (t, 3H).

### Step B: Preparation of ethyl 3-[[[2-(1,1-dimethylethyl)-5-ethyl-2H-1,2,3-triazol-4-yl]carbonyl]amino]benzoate

The title product, a compound of the present invention, was prepared from ethyl 2-(1,1-dimethylethyl)-5-ethyl-2H-1,2,3-triazole-4-carboxylate (i.e. the first eluted product of Step A) following a procedure analogous to Step C of Example 26.

¹H NMR (CDCl₃): δ 8.62 (br s, 1H), 8.14 (d, 1H), 8.09 (s, 1H), 7.81 (d, 1H), 7.44 (t, 1H), 4.80 (m, 1H), 4.40 (q, 2H), 3.04 (q, 2H), 1.70 (s, 9H), 1.41 (t, 3H), 1.31 (t, 3H).

### EXAMPLE 29

### Preparation of 2-(1,1-dimethylethyl)-5-ethyl-N-[3-[(ethylamino)carbonyl]phenyl]-2H-1,2,3-triazole-4-carboxamide (Compound 365)

The title product, a compound of the present invention, was prepared from ethyl 3-[[[2-(1,1-dimethylethyl)-5-ethyl-2H-1,2,3-triazol-4-yl]carbonyl]amino]benzoate (i.e. the product of Step B of Example 28) following a procedure analogous to Example 27.

¹H NMR (CDCl₃) δ 8.72 (br s, 1H), 8.06 (s, 1H), 7.88 (d, 1H), 7.53 (d, 1H), 7.36 (t, 1H), 6.71 (br s, 1H), 3.47 (q, 2H), 3.02 (q, 2H), 1.68 (s, 9H), 1.31 (t, 3H), 1.23 (t, 3H).

### EXAMPLE 30

### Preparation of 3-(1,1-dimethylethyl)-1-ethyl-N-[3-[(ethylamino)carbonyl]phenyl]-1H-1,2,4-triazole-5-carboxamide (Compound 446)

### Step A: Preparation of ethyl 5-(1,1-dimethylethyl)-1H-1,2,4-triazole-3-carboxylate

A solution of ethyl ethoxyiminoacetate (15.3 g, 106 mmol) and 2,2-dimethylpropanoic acid hydrazide (11.7 g, 101 mmol) in dichloromethane (320 mL) was stirred at room temperature for 48 h. The reaction mixture was concentrated in vacuo to leave a residue (about 75 mL), which was diluted with hexane (75 mL) to form a white solid precipitate. The solid was collected, washed with hexane, and dried to give 2,2-dimethylpropanoic acid 2-(2-ethoxy-1-imino-2-oxoethyl)hydrazide (5.3 g) as an intermediate, which was heated neat at 200° C. under nitrogen for 25 minutes. The reaction mixture was then cooled, and benzene (25 mL) was added. The mixture was concentrated to provide a crude solid, which was collected by filtration and then triturated with ether to provide the title compound as a yellow powder (2.2 g).

¹H NMR (CDCl₃) δ 4.21 (q, 2H), 1.41 (s, 9H), 1.34 (t, 3H).

### Step B: Preparation of ethyl 3-(1,1-dimethylethyl)-1-ethyl-1H-1,2,4-triazole-5-carboxylate

A slurry of ethyl 5-(1,1-dimethylethyl)-1H-1,2,4-triazole-3-carboxylate (i.e. the product of Step A) (2.44 g, 12.4 mmol), iodoethane (2.52 g, 16.2 mmol) and potassium carbonate (3.43 g, 25 mmol) in N,N-dimethylformamide (anhydrous, 12 mL) was stirred for 24 h at 70° C. under nitrogen. The reaction mixture was then diluted with water (100 mL) and extracted with ethyl acetate (1×80 mL, then 2×40 mL). The combined organic layers were washed with water (80 mL) and filtered through Celite® diatomaceous filter aid. The solvent was removed in vacuo to give a crude solid. This was purified by column chromatography (ethyl acetate—hexane) to give the title compound as a solid (1.84 g).

¹H NMR (CDCl₃) δ 4.58 (q, 2H), 4.47 (q, 3H), 1.46 (t, 3H), 1.43 (t, 3H), 1.39 (s, 9H).

### Step C: Preparation of 3-(1,1-dimethylethyl)-1-ethyl-N-[3-[(ethylamino)carbonyl]-phenyl]-1H-1,2,4-triazole-5-carboxamide

A solution of ethyl 3-(1,1-dimethylethyl)-1-ethyl-1H-1,2,4-triazole-5-carboxylate (i.e. the product of Step B) (0.30 g, 1.33 mmol), 3-amino-N-ethyl-benzamide (0.22 g, 1.33 mmol) and 4-(dimethylamino)pyridine (20 mg) in α,α,α-trifluorotoluene (0.2 mL) was heated to 210° C. in a microwave reactor for 30 minutes. The solvent was removed in vacuo to leave a crude solid. This was purified by column chromatography (ethyl acetate—hexane) to provide the title product, a compound of the present invention, as a solid (90 mg).

¹H NMR (CDCl₃) δ 9.30 (br s, 1H), 8.05 (s, 1H), 7.85 (d, 1H), 7.55 (d, 1H), 7.42 (t, 1H), 6.20 (br s, 1H), 4.68 (q, 2H), 3.50 (m, 2H), 1.51 (t, 3H), 1.39 (s, 9H), 1.27 (t, 3H).

By the procedures described herein together with methods known in the art, the following compounds of Tables 1 to 16 can be prepared. The following abbreviations are used in the Tables which follow: t means tertiary, s means secondary, n means normal, i means iso, c means cyclo, Me means methyl, Et means ethyl, Pr means propyl, i-Pr means isopropyl, Bu means butyl, Ph means phenyl, OMe means methoxy, OEt means ethoxy, SMe means methylthio, SEt means ethylthio, CN means cyano, NO₂ means nitro, TMS means trimethylsilyl, S(O)Me means methylsulfinyl, and S(O)₂Me means methylsulfonyl. Furthermore, 1-pyrrolyl means —N(—(CH₂)₅—), 3-pyrrolin-1-yl means —N(—CH₂CH═CHCH₂—), and 4-morpholinyl means —N(—(CH₂)₂O(CH₂)₂—).

TABLE 1

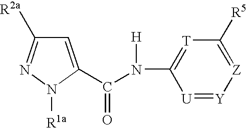

| | |
|---|---|
| R⁵ | R⁵ |

R^{1a} is Et; R^{2a} is tert-Bu T, U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et; R^{2a} is tert-Bu ; T , U and Y are CH ; Z is CF

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CHHD 2)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; U is CF ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; T is CF ; T, Y and Z are CH

| | |
|---|---|
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH≡CH₂ |
| C(O)NMe₂ | C(O)NMeCH₂CH≡CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

R^{1a} is Me ; R^{2a} is tert-Bu ; U is CF ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NMe(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Me ; R^{2a} is tert-Bu ; T, U, Y and Z are CH

| | |
|---|---|
| C(NOH)CH₃ | S(O)₂NHMe |
| C(NOH)CH₂CH₃ | S(O)₂NMe₂ |
| C(NOH)CH₂Cl | S(O)₂NHEt |
| C(NOH)CH₂Br | C(O)-(1-pyrrolyl) |
| C(NOCH₃)CH₂CH₃ | S(O)₂NEt₂ |
| C(NOH)CH₂CH₂Cl | S(O)₂NH(CH₂)₂CH₃ |
| CN | S(O)₂NH(CH₂)₃CH₃ |
| C(O)-(3-pyrrolin-1-yl) | |

R^{1a} is Et ; R^{2a} is isopropyl ; T, U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is isopropyl ; U is CF ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is cyclopropyl ; T, U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NMe |
| C(O)CH₂CH₃ | S(O)₂NMe₂ |
| C(NOH)CH₃ | S(O)₂NHEt |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)_{3CH}₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe2 |
| SCH₂CH₃ | OS(O)₂CH₃ |
| OS(O)₂CH₂Cl | OP(O)(OMe)₂ |

R^{1a} is CH₂CH₂F ; R^{2a} is tert-Bu ; T, U, Y and Z are CH

| | |
|---|---|
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

R^{1a} is CH₂CH₂F ; R^{2a} is tert-Bu ; U is CF ; T, Y and Z are CH

| | |
|---|---|
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

R^{1a} is CH₂CF₃; R^{2a} is tert-Bu ; U is CF ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is CH₂CF₃ ; R^{2a} is tert-Bu ; T, U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |

R^{1a} is CH₂CF₃ ; R^{2a} is tert-Bu ; T, U, Y and Z are CH

| | |
|---|---|
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF2H | C(O)NHMe |
| C(O)CH₂CH₂Cl | C(O)NMe₂ |
| OS(O)₂CH₂CH₃ | OP(O)Me(OMe) |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Me ; R^{2a} is tert-Bu ; U is N ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yI) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| C(NOH)CH₂Br | C(O)NH₂ |
| C(O)CF2H | C(O)NHMe |
| C(O)CH₂CH₂Cl | C(O)NMe₂ |
| OS(O)₂CH₂CH₃ | OP(O)Me(OMe) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; U is N ; T, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C═CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH↑CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; T is N ; U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; T is N ; U, Y and Z are CH

| | |
|---|---|
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |

R^{1a} is Et ; R^{2a} is tert-Bu ; T , U and Y are CH ; Z is N

| | |
|---|---|
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C57 CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

R^{1a} is CH₂CH_{3 ; R}^{2a} is Si(CH₃)₃ ; T, U, Y and Z are CH

| | |
|---|---|
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

TABLE 2

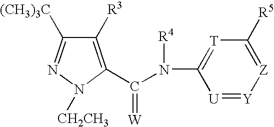

| R³ | R⁴ | W | R⁵ | T | U | Y | Z |
|---|---|---|---|---|---|---|---|
| F | H | O | C(O)NMe₂ | CH | CH | CH | CH |
| Me | H | O | C(O)NHEt | CH | CF | CH | CH |
| Et | H | O | C(O)NMe₂ | CH | N | CH | CH |
| H | Me | O | C(O)NMe₂ | CH | CH | CH | CH |
| H | Et | O | C(O)NHEt | CH | CF | CH | CH |
| H | C(O)Et | O | C(O)NMe₂ | CH | CH | CH | CH |
| H | C(O)O-n-Pr | O | C(O)NHEt | CH | N | CH | CH |
| H | CH₂OMe | O | C(O)NMe₂ | CH | CH | CH | CH |
| H | CH₂S-n-Bu | O | C(O)NHEt | CH | CF | CH | CH |
| H | H | S | C(O)NMe₂ | CH | CH | CH | CH |
| H | H | S | C(O)NHEt | CH | CF | CH | CH |
| H | H | O | C(O)NMe₂ | N | N | CH | CH |
| H | H | O | C(O)NHEt | CH | N | CH | N |
| H | H | O | C(O)NMe₂ | CH | N | N | CH |
| H | H | O | C(O)NHEt | N | N | CH | N |
| H | H | O | C(O)NMe₂ | CH | CCF₃ | CH | CH |
| H | H | O | C(O)NHEt | CH | COEt | CH | CH |
| H | H | O | C(O)NMe₂ | CH | COCF₂H | CH | CH |
| H | H | O | C(O)NHEt | CH | CMe | CH | CH |
| H | H | O | C(O)NMe₂ | CH | CEt | CH | CH |
| H | H | O | C(O)NHEt | CH | CSMe | CH | CH |
| H | H | O | C(O)NMe₂ | CH | CSEt | CH | CH |
| H | H | O | C(O)NHEt | CH | COMe | CH | CH |
| H | H | O | C(O)NMe₂ | CH | CH | CF | CH |
| H | H | O | C(O)NHEt | CH | CH | CCH3 | CH |
| H | H | O | C(O)OEt | CCF₃ | CH | CH | CH |
| H | H | O | C(O)OMe | CSMe | CH | CH | CH |
| H | H | O | C(O)OEt | COCF₂H | CH | CH | CH |
| H | H | O | C(O)OMe | CMe | CH | CH | CH |
| H | H | O | C(O)OEt | COMe | CH | CH | CH |
| H | H | O | C(O)OMe | CH | CH | CH | COMe |
| H | H | O | C(O)OEt | CH | CH | CH | COCF₂H |
| H | H | O | C(O)OMe | CH | CH | CH | CMe |
| H | H | O | C(O)OEt | CH | CH | CH | CCF3 |
| H | H | O | C(O)OMe | CH | CH | CH | CSMe |

| | | | | | | |
|---|---|---|---|---|---|---|
| H | H | O | —C(O)NHCH₂— | CH | CH | CH |
| H | H | O | —C(O)NMeCH₂— | CH | CH | CH |
| H | H | O | —C(O)NEtCH₂— | CH | CH | CH |
| H | H | O | —C(O)N(cyclo-Pr)CH₂— | CH | CH | CH |
| H | H | O | —C(O)N(i-Pr)CH₂— | CH | CH | CH |
| H | H | O | —C(O)NMeCH₂— | N | CH | CH |
| H | H | O | —C(O)NEtCH₂— | CF | CH | CH |
| H | H | O | —C(O)NMeCH₂CH₂— | CH | CH | CH |

TABLE 3

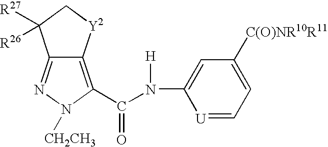

| R²⁶ | R²⁷ | Y² | U | R¹⁰ | R¹¹ |
|---|---|---|---|---|---|
| Me | Me | CH₂ | CH | Me | Me |
| Me | Me | O | CF | H | Et |
| Me | Me | S | N | Me | Me |
| Me | Me | NH | CH | H | Et |
| Me | Me | NCH₃ | CF | Me | Me |
| Me | H | CH₂ | CF | H | Et |
| Et | H | CH₂ | N | Me | Me |
| Et | Me | CH₂ | CH | H | Et |
| Et | Et | CH₂ | CF | Me | Me |
| Me | Me | CH | CH | H | Et |

TABLE 4

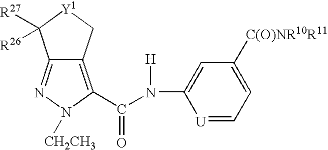

| R²⁶ | R²⁷ | Y¹ | U | R¹⁰ | R¹¹ |
|---|---|---|---|---|---|
| Me | Me | CH₂ | CH | Me | Me |
| Me | Me | O | CF | H | Et |
| Me | Me | S | N | Me | Me |
| Me | Me | NH | CH | H | Et |
| Me | Me | NCH₃ | CF | Me | Me |
| Me | H | CH₂ | CF | H | Et |
| Et | H | CH₂ | N | Me | Me |
| Et | Me | CH₂ | CH | H | Et |
| Et | Et | CH₂ | CF | Me | Me |
| Me | Me | CH | CH | H | Et |

TABLE 5

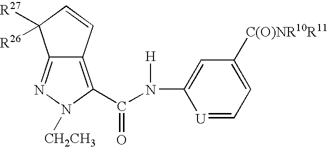

| R²⁶ | R²⁷ | U | R¹⁰ | R¹¹ |
|---|---|---|---|---|
| Me | Me | CH | Me | Me |
| Me | Me | CF | H | Et |
| Me | Me | N | Me | Me |
| Me | Me | CH | H | Et |
| Me | Me | CF | Me | Me |
| Me | H | CF | H | Et |
| Et | H | N | Me | Me |
| Et | Me | CH | H | Et |
| Et | Et | CF | Me | Me |
| Me | Me | N | H | Et |

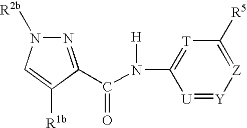

| R⁵ | R⁵ |
|---|---|
| R^{1b} is Et ; R^{2b} is tert-Bu ; T, U, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C═CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt2 |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Me R^{2b} is tert-Bu ; T, U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1b} is Et ; R^{2b} is tert-Bu ; U is N ; T, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)CH₃ |
| CO₂CH₂CH═CH₂ | S(O)(CH₂)₂CH₂Cl |
| CO₂CH₂C═CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH93CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Et ; R^{2b} is tert-Bu ; T is N ; U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂Cl |
| C(NOH)CH₃ | C(O)NMeCH₂CH₂Cl |
| C(NOH)CH₂CH₃ | C(O)NHMe(CH₂)₂CH₃ |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

TABLE 7

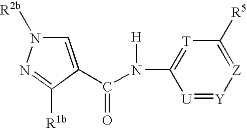

| R⁵ | R⁵ |
|---|---|
| R^{1b} is Et ; R^{2b} is tert-Bu ; T, U, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOII)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| C(NOCH₃)CH₂CH₃ | C()NEt₂ |
| R^{1b} is Et ; R^{2b} is tert-Bu ; U is CF ; T, Y and Z are CH | |
| CO₂Et | SCH₂CH₂F |
| CO₂CH₃ | S(CH₂)₂CH₃ |
| C2O₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Me ; R^{2b} is tert-Bu ; T, U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂CH≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1b} is Me ; R^{2b} is tert-Bu ; U is CF ; T, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1b} is Et ; R^{2b} is tert-Bu ; U is N ; T, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH2 |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Et ; R^{2b} is tert-Bu ; T is N ; U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C═CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

TABLE 8

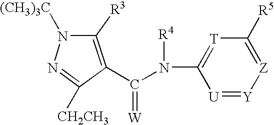

| R³ | R⁴ | W | R⁵ | T | U | Y | Z |
|---|---|---|---|---|---|---|---|
| F | H | O | C(O)NMe₂ | CH | CH | CH | CH |
| Me | H | O | C(O)NHEt | CH | CF | CH | CH |
| Et | H | O | C(O)NMe₂ | CH | N | CH | CH |
| H | Me | O | C(O)NMe₂ | CH | CH | CH | CH |
| H | C(O)Me | O | C(O)NMe₂ | CH | CH | CII | CH |
| H | C(O)OEt | O | C(O)NHEt | CH | N | CH | CH |
| H | CH₂OMe | O | C(O)NMe₂ | CH | CH | CH | CH |
| H | H | S | C(O)NMe₂ | CH | CH | CH | CH |
| H | H | S | C(O)NHEt | CH | CF | CH | CH |
| H | H | O | C(O)NMe₂ | N | N | CH | CH |
| H | H | O | C(O)NMe₂ | CH | CCF₃ | CH | CH |
| H | H | O | C(O)NHEt | CH | CMe | CH | CH |
| H | H | O | C(O)NMe₂ | CH | CH | CF | CH |
| H | H | O | C(O)NHEt | CH | CH | CCH₃ | CH |
| H | H | O | C(O)OMe | CMe | CH | CH | CH |
| H | H | O | C(O)OEt | COMe | CH | CH | CH |
| H | H | O | C(O)OMe | CH | CH | CH | COMe |
| H | H | O | C(O)OMe | CH | CH | CH | CMe |

TABLE 9

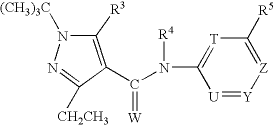

| R⁵ | R⁵ |
|---|---|
| R^{1b} is Et ; R^{2a} is tert-Bu ; T, U, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₂CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Et; R^{2a} is tert-Bu ; U is CF ; T, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C≡CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Me ; R^{2a} is tert-Bu ; T, U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂(CH₂)₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1b} is Me ; R^{2a} is tert-Bu ; U is CF; T, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂C1 |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1b} is Et ; R^{2a} is tert-Bu ; U is N ; T, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C═CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1b} is Et ; R^{2a} is tert-Bu ; T is N ; U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

TABLE 10

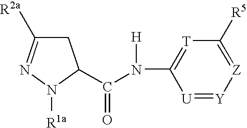

| R⁵ | R⁵ |
|---|---|
| R^{1a} is Et ; R^{2a} is tert-Bu ; T, U, Y and Z are CH | |
| CO₂Et | SCH₂CH₂F |
| CO₂CH₃ | S(CH₂)₂CH₃ |
| CO₂(CH₂)₂CH₃ | S(CH₂)₂CH₂F |
| CO₂(CH₂)₂CH₂Cl | S(CH₂)₂CH₂Cl |
| CO₂(CH₂)₂CH₂F | S(O)CH₂CH₃ |
| CO₂CH₂CH₂F | S(O)₂CH₂CH₃ |
| CO₂CH₂CH₂Cl | S(O)(CH₂)₂CH₃ |
| CO₂(CH₂)₃CH₃ | S(O)₂(CH₂)₂CH₃ |
| CO₂CH₂CH═CH₂ | S(O)(CH₂)₂CH₂Cl |
| CO₂CH₂C═CH | S(O)₂(CH₂)₂CH₂Cl |
| CO₂-cyclopropyl | S(O)CH₂CH₂F |
| CO₂-cyclopentyl | S(O)₂CH₂CH₂F |
| CO₂CH₂OCH₃ | S(O)CH₂CH₂Cl |
| CO₂CH₂SCH₃ | S(O)₂CH₂CH₂Cl |
| C(O)CH₃ | S(O)₂NHMe |
| C(O)CH₂CH₃ | S(O)₂NMe₂ |
| C(NOH)CH₃ | S(O)₂NHEt |
| C(NOH)CH₂CH₃ | S(O)₂NEt₂ |
| C(O)CH₂Cl | S(O)₂NH(CH₂)₂CH₃ |
| C(O)CH₂Br | S(O)₂NH(CH₂)₃CH₃ |
| C(NOH)CH₂Cl | C(O)NHEt |
| C(NOH)CH₂Br | C(O)NH₂ |
| C(O)CF₂H | C(O)NHMe |
| C(O)CH₂CH₂Cl | C(O)NMe₂ |
| OS(O)₂CH₂CH₃ | OP(O)Me(OMe) |
| C(NOCH₃)CH₂CH₃ | C(O)NEt₂ |
| C(NOH)CH₂CH₂Cl | C(O)NHCH₂CH₂F |
| CN | C(O)NHCH₂CH₂Cl |
| OCH₃ | C(O)NH(CH₂)₂CH₃ |
| OCH₂CH₃ | C(O)NH(CH₂)₂CH₂F |
| O(CH₂)₂CH₃ | C(O)NH(CH₂)₂CH₂Cl |
| O(CH₂)₃CH₃ | C(O)NH(CH₂)₃CH₃ |
| OCH₂CH₂F | C(O)NH(CH₂)₃CH₂F |
| OCH₂CH₂Cl | C(O)NH(CH₂)₃CH₂Cl |
| O(CH₂)₂CH₂F | C(O)NMeCH₂CH₂F |
| O(CH₂)₂CH₂Cl | C(O)NMeCH₂CH₂Cl |
| O(CH₂)₃CH₂Cl | C(O)NMeOMe |
| O(CH₂)₃CH₂F | C(O)NHCH₂C≡CH |
| O-cyclopentyl | C(O)NMeCH₂C≡CH |
| O-cyclopropyl | C(O)NHCH₂CH═CH₂ |
| OCH₂OCH₃ | C(O)NMeCH₂CH═CH₂ |
| OCH₂SCH₃ | C(O)NMeEt |
| SCH₃ | C(O)-(3-pyrrolin-1-yl) |
| S(O)CH₃ | C(O)-(4-morpholinyl) |
| S(O)₂CH₃ | C(O)NMe(CH₂)₂CH₃ |
| SCF₃ | C(O)-(1-pyrrolyl) |
| S(O)CF₃ | C(S)NHEt |
| S(O)₂CF₃ | C(S)NMe₂ |
| SCH₂CH₃ | OS(O)₂CH₃ |
| OS(O)₂CH₂Cl | OP(O)(OMe)₂ |
| R^{1a} is Et ; R^{2a} is tert-Bu ; U is CF ; T, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C≡CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C═CH |
| C(O)NH₂ | C(O)NMeCH₂C═CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1a} is Et ; R^{2a} is tert-Bu ; U is N ; T, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |
| R^{1a}is Et ; R^{2a} is tert-Bu ; T is N ; U, Y and Z are CH | |
| CO₂Et | C(O)NH(CH₂)₂CH₂F |
| CO₂CH₃ | C(O)NH(CH₂)₂CH₂Cl |
| CO₂(CH₂)₂CH₃ | C(O)NH(CH₂)₃CH₃ |
| CO₂CH₂CH═CH₂ | C(O)NH(CH₂)₃CH₂F |
| CO₂CH₂C═CH | C(O)NH(CH₂)₃CH₂Cl |
| C(NOH)CH₃ | C(O)NMeCH₂CH₂Cl |
| C(NOH)CH₂CH₃ | C(O)NMeOMe |
| C(O)NHEt | C(O)NHCH₂C≡CH |
| C(O)NH₂ | C(O)NMeCH₂C≡CH |
| C(O)NHMe | C(O)NHCH₂CH═CH₂ |
| C(O)NMe₂ | C(O)NMeCH₂CH═CH₂ |
| C(O)NEt₂ | C(O)NMeEt |
| C(O)NHCH₂CH₂Cl | C(O)-(3-pyrrolin-1-yl) |
| C(O)NH(CH₂)₂CH₃ | C(O)-(1-pyrrolyl) |

TABLE 11

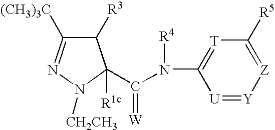

| R^{1c} | R³ | R⁴ | W | R⁵ | T | U | Y | Z |
|---|---|---|---|---|---|---|---|---|
| H | F | H | O | C(O)NMe₂ | CH | CH | CH | CH |
| H | Me | H | O | C(O)NHEt | CH | CF | CH | CH |
| H | Et | H | O | C(O)NMe₂ | CH | N | CH | CH |
| H | H | Me | O | C(O)NMe₂ | CH | CH | CH | CH |
| H | H | C(O)Me | O | C(O)NMe₂ | CH | CH | CH | CH |
| H | H | C(O)OEt | O | C(O)NHEt | CH | N | CH | CH |
| H | H | CH₂OMe | O | C(O)NMe₂ | CH | CH | CH | CH |
| H | H | H | S | C(O)NMe₂ | CH | CH | CH | CH |
| H | H | H | S | C(O)NHEt | CH | CF | CH | CH |
| H | H | H | O | C(O)NMe₂ | N | N | CH | CH |
| H | H | H | O | C(O)NMe₂ | CH | CCF₃ | CH | CH |
| H | H | H | O | C(O)NHEt | CH | CMe | CH | CH |
| H | H | H | O | C(O)NMe₂ | CH | CH | CF | CH |
| H | H | H | O | C(O)NHEt | CH | CH | CCH₃ | CH |
| H | H | H | O | C(O)OMe | CMe | CH | CH | CH |
| H | H | H | O | C(O)OEt | COMe | CH | CH | CH |
| H | H | H | O | C(O)OMe | CH | CH | CH | COMe |
| H | H | H | O | C(O)OMe | CH | CH | CH | CMe |

TABLE 12

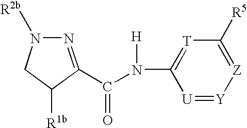

| | | | |
|---|---|---|---|
| R⁵ | R⁵ | R⁵ | R⁵ |

R^{1b} is Et; R^{2b} is tert-Bu; T, U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1b} is Et; R^{2b} is tert-Bu; U is N; T, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1b} is Et; R^{2b} is tert-Bu; T is N; U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

TABLE 13

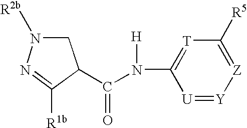

| | | | |
|---|---|---|---|
| R⁵ | R⁵ | R⁵ | R⁵ |

R^{1b} is Et; R^{2b} is tert-Bu; T, U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1b} is Et; R^{2b} is tert-Bu; U is CF; T, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1b} is Et; R^{2b} is tert-Bu; U is N; T, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1b} is Et; R^{2b} is tert-Bu; T is N; U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

TABLE 14

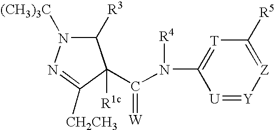

| R^{1c} | R³ | R⁴ | W | R⁵ | T | U | Y | Z |
|---|---|---|---|---|---|---|---|---|
| H | F | H | O | C(O)NMe₂ | CH | CH | CH | CH |
| H | Me | H | O | C(O)NHEt | CH | CF | CH | CH |
| H | H | Me | O | C(O)NMe₂ | CH | CH | CH | CH |
| H | H | C(O)Me | O | C(O)NMe₂ | CH | CH | CH | CH |
| H | H | H | S | C(O)NMe₂ | CH | CH | CH | CH |
| H | H | H | O | C(O)NMe₂ | N | N | CH | CH |
| H | H | H | O | C(O)NMe₂ | CH | CCF₃ | CH | CH |
| H | H | H | O | C(O)NHEt | CH | CMe | CH | CH |
| H | H | H | O | C(O)NMe₂ | CH | CH | CF | CH |
| H | H | H | O | C(O)NHEt | CH | CH | CCH₃ | CH |
| H | H | H | O | C(O)OMe | CMe | CH | CH | CH |
| H | H | H | O | C(O)OMe | CH | CH | CH | CMe |
| H | Me | H | O | C(O)-(3- | CH | CH | CH | CH |
| | | | | pyrrolin-1-yl) | | | | |
| H | H | C(O)OEt | O | C(O)- | CH | CF | CH | CH |
| | | | | (1-pyrrolyl) | | | | |

TABLE 15

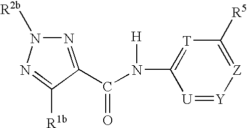

| | | | |
|---|---|---|---|
| R⁵ | R⁵ | R⁵ | R⁵ |

R^{1b} is Et; R^{2b} is tert-Bu; T, U, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1b} is Me; R^{2b} is tert-Bu; T, U, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1b} is Et; R^{2b} is tert-Bu; U is N; T, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1b} is Et; R^{2b} is tert-Bu; T is N; U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NHMe(CH₂)₂CH₃ | C(O)-(1-pyrrolyl) |

TABLE 16

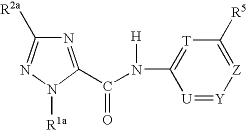

| | | | |
|---|---|---|---|
| R⁵ | R⁵ | R⁵ | R⁵ |

R^{1a} is Et; R^{2a} is tert-Bu; T, U, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1a} is Et; R^{2a} is tert-Bu; U is CF; T, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1a} is Me; R^{2a} is tert-Bu; T, U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1a} is Me; R^{2a} is tert-Bu; U is CF; T, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

R^{1a} is Et; R^{2a} is tert-Bu; U is N; T, Y and Z are CH

| | | | |
|---|---|---|---|
| CO₂Et | C(NOCH₃)CH₂CH₃ | SCH₂CH₂F | C(O)NEt₂ |
| CO₂CH₃ | C(NOH)CH₂CH₂Cl | S(CH₂)₂CH₃ | C(O)NHCH₂CH₂F |
| CO₂(CH₂)₂CH₃ | CN | S(CH₂)₂CH₂F | C(O)NHCH₂CH₂Cl |
| CO₂(CH₂)₂CH₂Cl | OCH₃ | S(CH₂)₂CH₂Cl | C(O)NH(CH₂)₂CH₃ |
| CO₂(CH₂)₂CH₂F | OCH₂CH₃ | S(O)CH₂CH₃ | C(O)NH(CH₂)₂CH₂F |
| CO₂CH₂CH₂F | O(CH₂)₂CH₃ | S(O)₂CH₂CH₃ | C(O)NH(CH₂)₂CH₂Cl |
| CO₂CH₂CH₂Cl | O(CH₂)₃CH₃ | S(O)(CH₂)₂CH₃ | C(O)NH(CH₂)₃CH₃ |
| CO₂(CH₂)₃CH₃ | OCH₂CH₂F | S(O)₂(CH₂)₂CH₃ | C(O)NH(CH₂)₃CH₂F |
| CO₂CH₂CH═CH₂ | OCH₂CH₂Cl | S(O)(CH₂)₂CH₂Cl | C(O)NH(CH₂)₃CH₂Cl |
| CO₂CH₂C≡CH | O(CH₂)₂CH₂F | S(O)₂(CH₂)₂CH₂Cl | C(O)NMeCH₂CH₂F |
| CO₂-cyclopropyl | O(CH₂)₂CH₂Cl | S(O)CH₂CH₂F | C(O)NMeCH₂CH₂Cl |
| CO₂-cyclopentyl | O(CH₂)₃CH₂Cl | S(O)₂CH₂CH₂F | C(O)NMeOMe |
| CO₂CH₂OCH₃ | O(CH₂)₃CH₂F | S(O)CH₂CH₂Cl | C(O)NHCH₂C≡CH |
| CO₂CH₂SCH₃ | O-cyclopentyl | S(O)₂CH₂CH₂Cl | C(O)NMeCH₂C≡CH |
| C(O)CH₃ | O-cyclopropyl | S(O)₂NHMe | C(O)NHCH₂CH═CH₂ |
| C(O)CH₂CH₃ | OCH₂OCH₃ | S(O)₂NMe₂ | C(O)NMeCH₂CH═CH₂ |
| C(NOH)CH₃ | OCH₂SCH₃ | S(O)₂NHEt | C(O)NMeEt |
| C(NOH)CH₂CH₃ | SCH₃ | S(O)₂NEt₂ | C(O)-(3-pyrrolin-1-yl) |
| C(O)CH₂Cl | S(O)CH₃ | S(O)₂NH(CH₂)₂CH₃ | C(O)-(4-morpholinyl) |
| C(O)CH₂Br | S(O)₂CH₃ | S(O)₂NH(CH₂)₃CH₃ | C(O)NMe(CH₂)₂CH₃ |
| C(NOH)CH₂Cl | SCF₃ | C(O)NHEt | C(O)-(1-pyrrolyl) |
| C(NOH)CH₂Br | S(O)CF₃ | C(O)NH₂ | C(S)NHEt |
| C(O)CF₂H | S(O)₂CF₃ | C(O)NHMe | C(S)NMe₂ |
| C(O)CH₂CH₂Cl | SCH₂CH₃ | C(O)NMe₂ | OS(O)₂CH₃ |
| OS(O)₂CH₂CH₃ | OS(O)₂CH₂Cl | OP(O)Me(OMe) | OP(O)(OMe)₂ |

R^{1a} is Et; R^{2a} is tert-Bu; T is N; U, Y and Z are CH

| | | | |
|---|---|---|---|
| CO₂Et | C(O)NHEt | C(O)NH(CH₂)₂CH₂F | C(O)NHCH₂C≡CH |
| CO₂CH₃ | C(O)NH₂ | C(O)NH(CH₂)₂CH₂Cl | C(O)NMeCH₂C≡CH |
| CO₂(CH₂)₂CH₃ | C(O)NHMe | C(O)NH(CH₂)₃CH₃ | C(O)NHCH₂CH═CH₂ |
| CO₂CH₂CH═CH₂ | C(O)NMe₂ | C(O)NH(CH₂)₃CH₂F | C(O)NMeCH₂CH═CH₂ |
| CO₂CH₂C≡CH | C(O)NEt₂ | C(O)NH(CH₂)₃CH₂Cl | C(O)NMeEt |
| C(NOH)CH₃ | C(O)NHCH₂CH₂Cl | C(O)NMeCH₂CH₂Cl | C(O)-(3-pyrrolin-1-yl) |
| C(NOH)CH₂CH₃ | C(O)NH(CH₂)₂CH₃ | C(O)NMeOMe | C(O)-(1-pyrrolyl) |

Formulation/Utility

Compounds of Formula I or Iz will generally be used as a formulation or composition with an agriculturally suitable carrier comprising at least one of a liquid diluent, a solid diluent or a surfactant. The formulation or composition ingredients are selected to be consistent with the physical properties of the active ingredient, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 5-90 | 0-94 | 1-15 |
| soluble Granules, Tablets and | | | |
| Powders. | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, and polyoxy-ethylene/polyoxypropylene block copolymers. Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol, benzyl and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; and Hance et al., Weed Control Handbook, 8th Ed., Blackwell Scientific Publications, Oxford, 1989.

In the following Examples, all percentages are by weight and all formulations are prepared in conventional ways. Compound numbers refer to compounds in Index Tables A-L.

### EXAMPLE A

High Strength Concentrate

| | |
|---|---|
| Compound 2 | 98.5% |
| silica aerogel | 0.5% |
| synthetic amorphous fine silica | 1.0%. |

### EXAMPLE B

Wettable Powder

| | |
|---|---|
| Compound 6 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0%. |

### EXAMPLE C

Granule

| | |
|---|---|
| Compound 156 | 10.0% |
| attapulgite granules (low volatile matter, | 90.0%. |
| 0.71/0.30 mm; U.S.S. No. 25-50 sieves) | |

### EXAMPLE D

Aqueous Suspension

| | |
|---|---|
| Compound 2 | 25.0% |
| hydrated attapulgite | 3.0% |
| crude calcium ligninsulfonate | 10.0% |
| sodium dihydrogen phosphate | 0.5% |
| water | 61.5%. |

### EXAMPLE E

Extruded Pellet

| | |
|---|---|
| Compound 6 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0%. |

Test results indicate that the compounds of the present invention are highly active preemergent and postemergent herbicides or plant growth regulants. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and/or broadleaf weeds within a crop/weed mixture. Compounds of this invention may show tolerance to important agronomic crops including, but not limited to, alfalfa, barley, cotton, wheat, rape, sugar beets, corn (maize), sorghum, soybeans, sunflower, rice, oats, peanuts, vegetables, tomato, potato and perennial plantation crops. Those skilled in the art will appreciate that not all compounds are equally effective against all weeds. Compounds of the invention are particularly useful for selective control of weeds in perennial plantation crops, transplanted rice, maize and cool-season cereal crops. Of particular note is the use of compounds of the invention for selective weed control in perennial plantation crops (also known as permanent crops) including: fruit trees such as citrus (e.g., orange, lemon, lime, grapefruit, tangerine), pome fruits (e.g., apple, pear, quince) and stone fruits (e.g., peach, nectarine, apricot, plum, cherry), nut trees (e.g., almond, hickory, pecan, walnut, cashew, chestnut, filbert, macademia, pistachio), forest trees such as hardwoods (e.g., eucalyptus, oak, maple, birch, ash) and softwoods (i.e. conifers such as fir, redwood, spruce, cedar, cypress, larch, hemlock, loblolly and other pines), banana, plantain, pineapple, hops, coffee, tea, cocoa, oilseed palm, rubber, sugarcane, grapes (e.g., Vitus vinifera, V. labrusca, V. rotundifolia), and perennial turf grasses (e.g., Kentucky bluegrass, St. Augustine grass, Kentucky fescue, Bermuda grass). Alternatively, the subject compounds are useful to modify plant growth. The formulated compounds can be applied to the soil, for example, as a treatment spray mixture, mixed with solid fertilizer or included in irrigation water.

Many of the compounds have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. One skilled in the art will recognize that the preferred combination of these selectivity factors within a compound or group of compounds can readily be determined by performing routine biological and/or biochemical assays.

As the compounds of the invention have both preemergent and postemergent herbicidal activity, to control undesired vegetation by killing or injuring the vegetation or reducing its growth, the compounds can be usefully applied by a variety of methods which can include banding, directed sprays, or broadcast applications that involve contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of Formula I or Iz is determined by a number of factors. These factors include: formulation selected, method of application, amount and type of vegetation present, growing conditions, etc. In general, a herbicidally effective amount of compounds of this invention is about 0.1 g/ha to 20 kg/ha, with a preferred range of about 1 g/ha to about 5000 g/ha and a more preferred range of about 4 to about 3000 g/ha. One skilled in the art can easily determine the herbicidally effective amount necessary for the desired level of weed control.

The compounds of Formula Iz (including Formula I) may be used in combination with other herbicides, insecticides, or fungicides, and other agricultural chemicals such as fertilizers. Other herbicides, insecticides and fungicides can include biological agents such as the herbicidal microbes Altemaria destruens, Colletotrichum gloesporiodes, Drechsiera monoceras (MTB-951) and Puccinia thlaspeos. Mixtures of compounds of Formula Iz (or I) with other herbicides can broaden the spectrum of activity against additional weed species, and suppress the proliferation of any resistant biotypes. Mixtures of compounds of Formula Iz (or I) with other herbicides can also provide greater than expected (i.e. synergistic) control of weeds and/or less than expected (i.e. safening) effect on crops. Therefore an aspect of the present invention relates to a herbicidal mixture comprising a herbicidally effective amount of a compound of Formula Iz and an effective amount of another herbicide. Of note is said herbicidal mixture wherein the compound of Formula Iz is a compound of Formula I. A mixture of one or more of the following other herbicides with a compound of Formula Iz may be particularly useful for weed control: acetochlor, acifluorfen and its sodium salt, aclonifen, acrolein (2-propenal), alachlor, alloxydim, Alternaria destruens, ametryn, amicarbazone, amidosulfuron, amitrole, ammonium sulfamate, anilofos, asulam, atrazine, azafenidin, azimsulfuron, beflubutamid, benazolin, benazolin-ethyl, benfluralin, benfuresate, bensulfuron-methyl, bensulide, bentazone, benzfendizone, benzobicyclon, benzofenap, bifenox, bilanafos, bispyribac and its sodium salt, bromacil, bromobutide, bromoxynil, bromoxynil octanoate, butachlor, butafenacil, butamifos, butralin, butroxydim butylate, cafenstrole, caloxydim (BAS 620H), carbetamide, carfentrazone-ethyl, catechin, chlomethoxyfen, chloramben, chlorbromuron, chlorflurenol-methyl, chloridazon, chlorimuron-ethyl, chlomitrofen, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, clopyralid-olamine, cloransulam-methyl, Colletotrichum gloesporiodes, cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycola mmonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethipin, dimethylarsinic acid and its sodium salt, dinitramine, dinoterb, diphenamid, diquat dibromide, dithiopyr, diuron, DNOC, Drechsiera monoceras, endothal, EPTC, esprocarb, ethalfluralin, ethametsulfuron-methyl, ethofumesate, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, fluchloralin, flufenacet, flufenpyr-ethyl, flumetsulam, flumiclorac-pentyl, flumioxazin, fluometuron, fluoroglycofen-ethyl, flupoxam, flupyrsulfuron-methyl and its sodium salt, fluridone, flurochloridone, fluroxypyr, flurtamone, fluthiacet-methyl, fomesafen, foramsulfuron, fosamine-ammonium, furilazole, glufosinate and its salts such as particularly glufosinate-ammonium, glyphosate and its salts such as particularly glyphosate-ammonium, glyphosate-isopropylammonium, glyphosate-sodium, glyphosate-potassium and glyphosate-trimesium, halosulfuron-methyl, haloxyfop-etotyl, haloxyfop-methyl, hexazinone, imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, lactofen, lenacil, linuron, maleic hydrazide, MCPA and its dimethylammonium, potassium and sodium salts, MCPA-isoctyl, MCPB and its sodium salt, MCPB-ethyl, mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, methyl [[[1-[5-[2-chloro-4-(trifluoromethyl)phenoxy]-2-nitrophenyl]-2-methoxyethylidene]amino]oxy]-acetate (AKH-7088), methyl 5-[[[[(4,6-dimethyl-2-pyrimidinyl)amino]carbonyl]amino]-sulfonyl]-1-(2-pyridinyl)-1H-pyrazole-4-carboxylate (NC-330), metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, oryzalin, oxadiargyl, oxadiazon, oxasulfuron, oxaziclomefone, oxyfluorfen, paraquat dichloride, pebulate, pendimethalin, penoxsulam, pentanochlor, pentoxazone, perfluidone, pethoxamid, phenmedipham, picloram, picloram-potassium, picolinafen, piperofos, pretilachlor, primisulfuron-methyl, prodiamine, profoxydim (BAS625H, 2-[1-[[2-(4-chlorophenoxy)propoxy]imino]butyl]-3-hydroxy-5-(tetrahydro-2H-thiopyran-3-yl)-2-cyclohexen-1-one), prometon, prometryn, propachlor, propanil, propaquizafop, propazine, propham, propisochlor, propoxycarbazone, propyzamide, prosulfocarb, prosulfuron, Puccinia thlaspeos, pyraflufen-ethyl, pyrazolynate, pyrazoxyfen, pyrazosulfuron-ethyl, pyribenzoxim, pyributicarb, pyridate, pyriftalid, pyriminobac-methyl, pyrithiobac, pyrithiobac-sodium, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tepraloxydim, terbacil, terbumeton, terbuthylazine, terbutryn, thenylchlor, thiafluamide (BAY 11390), thiazopyr, thifensulfuron-methyl, thiobencarb, tiocarbazil, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Combinations of compounds of the invention with other herbicides can result in a greater-than-additive (i.e. synergistic) effect on weeds and/or a less-than-additive effect (i.e. safening) on crops or other desirable plants. For example, combination of a sulfonylurea herbicide such as thifensulfuron-methyl and tribenuron-methyl with a compound of the invention can reduce phytotoxicity to certain crops.

In certain instances, combinations with other herbicides having a similar spectrum of control but a different mode of action will be particularly advantageous for preventing the development of resistant weeds.

Preferred for better control of undesired vegetation (e.g., lower use rate such as from synergism, broader spectrum of weeds controlled, or enhanced crop safety) or for preventing the development of resistant weeds is a herbicidal mixture of a compound of Formula Iz (including Formula I) with an other herbicide selected from the group consisting of atrazine, bromacil, diuron, hexazinone, terbacil, glyphosate (particularly glyphosate-ammonium, glyphosate-isopropylammonium, glyphosate-sodium, glyphosate-potassium, glyphosate-trimesium), glufosinate (particularly glufosinate-ammonium), rimsulfuron, metsulfuron-methyl, sulfometuron-methyl, ametryn and paraquat. Specifically preferred mixtures (wherein compound A is N-[3-[(dimethylamino)carbonyl]phenyl]-3-(1,1-dimethylethyl)-1-methyl-1H-pyrazole-5-carboxamide (Formula Iz wherein J is J-1, R^{1a} is Me, R^{2a} is t-Bu, R³ is H, W is 0, R⁴ is H, T, U, Y and Z are CH, and R⁵ is C(O)NMe₂); other compound numbers refer to compounds in Index Tables A-L) are selected from the group: compound 2 and atrazine; compound 6 and atrazine; compound 14 and atrazine; compound 115 and atrazine; compound 152 and atrazine; compound 156 and atrazine; compound 162 and atrazine; compound 193 and atrazine; compound 222 and atrazine; compound A and atrazine, compound 2 and bromacil; compound 6 and bromacil; compound 14 and bromacil; compound 115 and bromacil; compound 152 and bromacil; compound 156 and bromacil; compound 162 and bromacil; compound 193 and bromacil; compound 222 and bromacil; compound A and bromacil; compound 2 and diuron; compound 6 and diuron; compound 14 and diuron; compound 115 and diuron; compound 152 and diuron; compound 156 and diuron; compound 162 and diuron; compound 193 and diuron; compound 222 and diuron; compound A and diuron; compound 2 and hexazinone; compound 6 and hexazinone; compound 14 and hexazinone; compound 115 and hexazinone; compound 152 and hexazinone; compound 156 and hexazinone; compound 162 and hexazinone; compound 193 and hexazinone; compound 222 and hexazinone; compound A and hexazinone; compound 2 and terbacil; compound 6 and terbacil; compound 14 and terbacil; compound 115 and terbacil; compound 152 and terbacil; compound 156 and terbacil; compound 162 and terbacil; compound 193 and terbacil; compound 222 and terbacil; compound A and terbacil; compound 2 and glyphosate; compound 6 and glyphosate; compound 14 and glyphosate; compound 115 and glyphosate; compound 152 and glyphosate; compound 156 and glyphosate; compound 162 and glyphosate; compound 193 and glyphosate; compound 222 and glyphosate; compound A and glyphosate; compound 2 and glufosinate; compound 6 and glufosinate; compound 14 and glufosinate; compound 115 and glufosinate; compound 152 and glufosinate; compound 156 and glufosinate; compound 162 and glufosinate; compound 193 and glufosinate; compound 222 and glufosinate; compound A and glufosinate; compound 2 and rimsulfuron; compound 6 and rimsulfuron; compound 14 and rimsulfuron; compound 115 and rimsulfuron; compound 152 and rimsulfuron; compound 156 and rimsulfuron; compound 162 and rimsulfuron; compound 193 and rimsulfuron; compound 222 and rimsulfuron; compound A and rimsulfuron; compound 2 and metsulfuron-methyl; compound 6 and metsulfuron-methyl; compound 14 and metsulfuron-methyl; compound 115 and metsulfuron-methyl; compound 152 and metsulfuron-methyl; compound 156 and metsulfuron-methyl; compound 162 and metsulfuron-methyl; compound 193 and metsulfuron-methyl; compound 222 and metsulfuron-methyl; compound A and metsulfuron-methyl; compound 2 and sulfometuron-methyl; compound 6 and sulfometuron-methyl; compound 14 and sulfometuron-methyl; compound 115 and sulfometuron-methyl; compound 152 and sulfometuron-methyl; compound 156 and sulfometuron-methyl; compound 162 and sulfometuron-methyl; compound 193 and sulfometuron-methyl; compound 222 and sulfometuron-methyl; compound A and sulfometuron-methyl; compound 2 and ametryn; compound 6 and ametryn; compound 14 and ametryn; compound 115 and ametryn; compound 152 and ametryn; compound 156 and ametryn; compound 162 and ametryn; compound 193 and ametryn; compound 222 and ametryn; compound A and ametryn; compound 2 and paraquat; compound 6 and paraquat; compound 14 and paraquat; compound 115 and paraquat; compound 152 and paraquat; compound 156 and paraquat; compound 162 and paraquat; compound 193 and paraquat; compound 222 and paraquat; compound A and paraquat. Particularly preferred because of greater than additive (i.e. synergistic) efficacy on certain weeds are mixtures of compound 2 and diuron; compound 2 and terbacil; compound 6 and atrazine; compound 6 and diuron; compound 6 and hexazinone; and compound 6 and terbacil. Herbicidally effective amounts of compounds of Formula Iz (including Formula I) as well as herbicidally effective amounts of other herbicides can be easily determined by one skilled in the art through simple experimentation. Synergistically effective amounts of these herbicidal compounds can likewise be easily determined.

Mixtures of compound 6 with diuron and hexazinone are especially notable for their synergistic activity in controlling Urochloa species (previously classified in genus Brachiaria) such as Urochloa decumbens (Staph) R. D. Webster, which is commonly known as Surinam grass or signal grass. U. decumbens is native to central Africa, but because it grows satisfactorily on poor soils, it has been planted in other tropical and subtropical regions for use as cattle forage. Unfortunately this species has subsequently become widespread and troublesome in many crops. As reported by R. A. Pitelli et al., “Brachiaria decumbens, a major exotic invasive plant in Brazil”, Weed Science Society of America Abstracts 2003, 43, 23, this species has become a major weed in forestry, citrus, sugarcane, horse pastures and roadsides as well as soybean, maize and cotton crops. Therefore a preferred embodiment of the present invention is a method for controlling the growth of undesired vegetation comprising Urochloa decumbens (Staph) R. D. Webster comprising contacting the vegetation or its environment with herbicidally effective amounts of the compound of Formula I (or Iz) which is N-[5-[(dimethylamino)carbonyl]-2-fluorophenyl]-3-(1,1-dimethylethyl)-1-ethyl-1H-pyrazole-5-carboxamide (compound 6) and at least one other herbicide selected from the group consisting of diuron and hexazinone. In said method compound 6 is typically applied at an application rate between about 60 and 600 g/ha, preferably between about 120 and 450 g/ha, and more preferably between about 240 and 360 g/ha; diuron is typically applied between about 250 and 2500 g/ha, preferably between about 500 and 2000 g/ha, and more preferably between about 960 and 1440 g/ha; and hexazinone is typically applied between about 100 and 600 g/ha, preferably between about 200 and 450 g/ha, more preferably between about 240 and 360 g/ha. Typical use rate ratios by weight for compound 6 to diuron (compound 6: diuron) are in the range of about 1:40 to 2:1, preferably about 1:17 to 1:1, and more preferably about 1:6 to about 1:3. Typical use rate ratios by weight for compound 6 to hexazinone are in the range of about 1:10 to 6:1, preferably about 1:4 to 2:1, and more preferably about 2:3 to 3:2.

Compounds of Formula Iz (including Formula I) can also be used in combination with herbicide safeners such as benoxacor, BCS (1-bromo-4-[(chloromethyl)sulfonyl]benzene), cloquintocet-mexyl, cyometrinil, dichlormid, 2-(dichloromethyl)-2-methyl-1,3-dioxolane (MG 191), fenchlorazole-ethyl, fenclorim, flurazole, fluxofenim, furilazole, isoxadifen-ethyl, mefenpyr-ethyl, methoxyphenone ((4-methoxy-3-methylphenyl)(3-methylphenyl)-methanone), naphthalic anhydride (1,8-naphthalic anhydride) and oxabetrinil to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of Formula Iz and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of Formula Iz wherein seed from which the crop is grown is treated with an antidotally effective amount of safener. Seed treatment with 1,8-naphthalic anhydride works well in a wide variety of crops such as maize, wheat, barley and sugarbeets. Of note is said method wherein the compound of Formula Iz is a compound of Formula I. Antidotally effective amounts of safeners can be easily determined by one skilled in the art through simple experimentation.

A compound of Formula Iz can thus be applied in admixture with other herbicides and/or herbicide safeners, in binary or multiple combinations in order to achieve optimal weed control spectrum and duration of weed control, suppress proliferation of resistant biotypes, benefit from synergy against particularly troublesome weeds and/or reduce injury to crops. Therefore an aspect of the present invention relates to a herbicidal mixture comprising a herbicidally effective amount of a compound of Formula Iz, an N-oxide or an agriculturally suitable salt thereof, and an effective amount of at least one additional active ingredient selected from the group consisting of an other herbicide and a herbicide safener. Typically the herbicidal mixture is applied in the form of a herbicidal composition comprising the herbicidal mixture and at least one of a surfactant, a solid diluent or liquid diluent. Related to this herbicidal mixture and herbicidal composition is a method for controlling the growth of undesired vegetation by applying a herbicidally effective amount of said herbicidal mixture or herbicidal composition to the locus of the undesired vegetation. Of note are said herbicidal mixture, herbicidal composition and method wherein the compound of Formula Iz is a compound of Formula I. Also of note are said herbicidal mixture, herbicidal composition and method wherein the compound of Formula Iz is selected from Formula Iz wherein: J is J-1, R^{1a} is Me, R^{2a} is t-Bu, R³ is H, W is O, R⁴ is H, T, U, Y and Z are CH, and R⁵ is C(O)NMe₂; J is J-1, R^{1a} is Me, R^{2a} is t-Bu, R³ is H, W is O, R⁴ is H, T, U, Y and Z are CH, and R⁵ is C(O)NHMe; J is J-1, R^{1a} is Me, R^{2a} is t-Bu, R³ is H, W is O, R⁴ is H, T, U, Y and Z are CH, and R⁵ is C(O)NH-n-Pr; J is J-1, R^{1a} is Me, R^{2a} is t-Bu, R³ is H, W is O, R⁴ is H, T, U, Y and Z are CH, and R⁵ is C(O)NEt₂; or J is J-1, R^{1a} is Me, R^{2a} is t-Bu, R³ is H, W is O, R⁴ is H, U, Y and Z are CH, T is N, and R⁵ is C(O)NEt₂ (or the pyridine N-oxide thereof, i.e. T is N(O)).

The following Tests demonstrate the control efficacy of the compounds of this invention against specific weeds. The weed control afforded by the compounds is not limited, however, to these species. See Index Tables A-L for compound descriptions. The following abbreviations are used in the Index Tables which follow: t means tertiary, s means secondary, n means normal, i means iso, c means cyclo, Me means methyl, Et means ethyl, Pr means propyl, i-Pr means isopropyl, Bu means butyl, Ph means phenyl, OMe means methoxy, OEt means ethoxy, SMe means methylthio, SEt means ethylthio, CN means cyano, NO₂ means nitro, TMS means trimethylsilyl, S(O)Me means methylsulfinyl, and S(O)₂Me means methylsulfonyl. The abbreviation “dec” indicates that the compound appeared to decompose on melting. The abbreviation “Ex.” stands for “Example” and is followed by a number indicating in which example the compound is prepared.

INDEX TABLE A

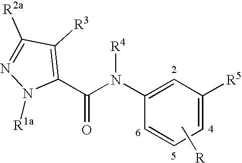

R can be one or more substituents; a dash (“-”) indicates no substituents.

| Compound | R^{1a} | R^{2a} | R³ | R⁴ | R | R⁵ | m.p. (° C.) |
|---|---|---|---|---|---|---|---|
| 1 | Et | t-Bu | H | H | 6-F | 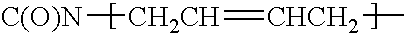 | * |
| 2 (Ex. 5, 6) | Et | t-Bu | H | H | 6-F | C(O)NHCH₂CH₃ | 188.5 |
| 3 | Et | t-Bu | H | H | 6-F | C(O)NHCH₂CH₂F | * |
| 4 | Et | t-Bu | H | H | 6-F | C(O)N(CH₂CH₃)₂ | * |
| 5 | Et | t-Bu | H | H | 6-F | C(O)NHCH₂CF₃ | * |
| 6 (Ex. 7) | Et | t-Bu | H | H | 6-F | C(O)N(CH₃)₂ | ** |
| 7 | Et | t-Bu | H | H | 6-F | C(O)NHCH₃ | 250-252 |
| 8 | Et | t-Bu | H | H | 2,4,5-tri-F | C(O)NHCH₂CH₃ | * |
| 9 | Et | t-Bu | H | H | 6-F | C(O)N(CH₃)CH₂CH═CH₂ | * |
| 10 | Et | t-Bu | H | H | 6-F | C(O)N(CH₃)CH₂C≡CH | * |
| 11 | Et | t-Bu | H | H | 6-F | C(O)NHCH₂CH₂OCH₃ | * |
| 12 | Et | t-Bu | H | H | 6-F | C(O)N(CH₃)CH₂CH(CH₃)₂ | * |
| 13 | Et | i-Pr | H | H | 6-F | C(O)NHCH₂CH₃ | * |
| 14 | Et | i-Pr | H | H | 6-F | C(O)N(CH₃)₂ | * |
| 15 | Et | i-Pr | H | H | 6-F | C(O)N(CH₂CH₃)₂ | * |
| 16 | Et | t-Bu | H | H | 6-F | C(O)N(CH₃)CH₂CH₃ | * |
| 17 | Et | t-Bu | H | H | 6-F | C(O)N(CH₃)CH₂CH₂CH₃ | * |
| 18 | Et | c-Pr | H | H | 6-F | C(O)N(CH₃)₂ | * |
| 19 | Et | c-Pr | H | H | 6-F | C(O)N(CH₂CH₃)₂ | * |
| 20 | Et | c-Pr | H | H | 6-F | C(O)NHCH₂CH₃ | * |
| 21 | Et | c-Pr | H | H | 6-F | C(O)N(CH₃)CH₂CH═CH₂ | * |
| 22 | Et | i-Bu | H | H | 6-F | C(O)N(CH₃)₂ | * |
| 23 | Et | i-Bu | H | H | 6-F | C(O)N(CH₂CH₃)₂ | * |
| 24 | Et | i-Bu | H | H | 6-F | C(O)NHCH₂CH₃ | * |
| 25 | Et | i-Bu | H | H | 6-F | C(O)N(CH₃)CH₂CH═CH₂ | * |
| 26 | Et | t-Bu | H | H | — | 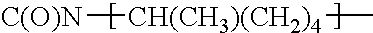 | 197-198 |
| 27 | Et | t-Bu | H | H | — | C(O)NHCH₂CH₃ | 176-179 |
| 28 | Et | t-Bu | H | H | — | C(O)N(CH₂CH₃)₂ | 153-155 |
| 29 | Et | t-Bu | H | H | — | C(O)NHCH₃ | 195-197 |
| 30 | CH₂CF₃ | t-Bu | H | H | — | C(O)NHCH₃ | 204-206 |
| 31 | CH₂CF₃ | t-Bu | H | H | — | C(O)NHCH₂CH₃ | * |
| 32 | CH₂CF₃ | t-Bu | H | H | — | C(O)N(CH₂CH₃)₂ | * |
| 33 | CH═CH₂ | t-Bu | H | H | — | C(O)NHCH₂CH₃ | * |
| 34 | CH═CH₂ | t-Bu | H | H | — | C(O)N(CH₂CH₃)₂ | * |
| 35 | CH₂CH₂F | t-Bu | H | H | — | C(O)NHCH₂CH₃ | * |
| 36 | CH₂CH₂F | t-Bu | H | H | — | C(O)N(CH₂CH₃)₂ | * |
| 37 | CH═CH₂ | t-Bu | H | H | — | C(O)NHCH₂CF₃ | * |
| 38 | CH═CH₂ | t-Bu | H | H | — | C(O)NHCH₂CH₂Cl | * |
| 39 | CH═CH₂ | t-Bu | H | H | — | C(O)NH(c-Pr) | * |
| 40 | n-Bu | t-Bu | H | H | — | C(O)N(CH₂CH₃)₂ | 154-156 |
| 41 | n-Bu | t-Bu | H | H | — | C(O)NHCH₂CH₃ | 139-141 |
| 42 | i-Bu | t-Bu | H | H | — | C(O)NHCH₂CH₃ | * |
| 43 (Ex. 3) | Et | t-Bu | H | H | — | C(O)NHCH₂CF₃ | ** |
| 44 | Et | t-Bu | H | H | — | C(O)NH(c-Pr) | * |
| 45 | Et | t-Bu | H | H | — | C(O)NHCH₂CH₂F | * |
| 46 | Et | t-Bu | H | H | — | C(O)NHCH₂CH═CH₂ | * |
| 47 | Et | t-Bu | H | H | — | C(O)NHCH₂C≡CH | * |
| 48 | Et | t-Bu | H | H | — | C(O)N(CH₃)CH₂CH₃ | * |
| 49 | Et | t-Bu | H | H | — | C(O)N(CH₃)₂ | * |
| 50 | CH₂C≡CH | t-Bu | H | H | — | C(O)NHCH₂CH₃ | * |
| 51 | Et | t-Bu | H | H | — | C(O)NH(i-Pr) | * |
| 52 | Et | t-Bu | H | H | | 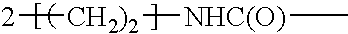 | * |
| 53 | Me | t-Bu | H | H | | 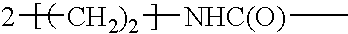 | * |
| 54 | Me | t-Bu | H | H | | 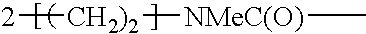 | * |
| 55 (Ex. 15) | Et | t-Bu | H | H | | 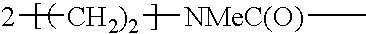 | ** |
| 56 | CH₂CH═CH₂ | t-Bu | H | H | — | C(O)NHCH₂CH₃ | * |
| 57 | n-Pr | t-Bu | H | H | — | C(O)NHCH₂CH₃ | 162-163 |
| 58 | Et | t-Bu | H | H | 4-F | C(O)NHCH₂CH₃ | * |
| 59 | Et | t-Bu | H | H | 4-F | C(O)N(CH₂CH₃)₂ | * |
| 60 | Et | t-Bu | H | H | 4-F | C(O)NHCH₂CH₂F | * |
| 61 | Et | t-Bu | H | H | — | C(O)NH(i-Bu) | * |
| 62 | Et | t-Bu | H | H | — | C(O)NHCH₂(c-Pr) | * |
| 63 | Et | t-Bu | H | H | | 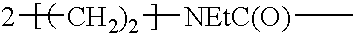 | * |
| 64 | Et | t-Bu | H | H | | 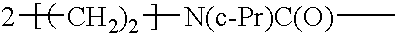 | * |
| 65 | Et | t-Bu | H | H | | 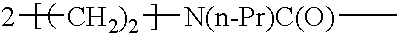 | * |
| 66 | Et | t-Bu | H | H | | 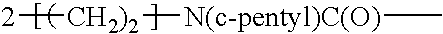 | * |
| 67 | Et | t-Bu | H | H | — | C(O)NHOCH₃ | * |
| 68 | Et | t-Bu | H | H | 4-CH₃ | C(O)NHCH₂CH₃ | * |
| 69 | Et | t-Bu | H | H | 4-CH₃ | C(O)N(CH₂CH₃)₂ | * |
| 70 (Ex. 4) | Et | t-Bu | H | H | 6-F | CO₂CH₂CH₃ | ** |
| 71 | Et | t-Bu | H | H | 6-F | CO₂(t-Bu) | * |
| 72 | Et | i-Bu | H | H | 6-F | CO₂CH₂CH₃ | * |
| 73 | Et | i-Pr | H | H | 6-F | CO₂CH₂CH₃ | * |
| 74 | Et | c-Pr | H | H | 6-F | CO₂CH₂CH₃ | * |
| 75 | Et | t-Bu | H | H | 6-F, 4-OCH₃ | CO₂CH₃ | * |
| 76 | Et | t-Bu | H | H | 6-F, 4-OCH₃ | CO₂CH₂CH₃ | * |
| 77 | Et | t-Bu | H | H | 6-F, 4-OCH₃ | CO₂(i-Pr) | * |
| 78 | CH═CH₂ | t-Bu | H | H | — | CO₂CH₂CH₃ | * |
| 79 | CH₂CH₂F | t-Bu | H | H | — | CO₂CH₂CH₃ | * |
| 80 (Ex. 1) | Et | t-Bu | H | H | — | CO₂CH₂CH₃ | ** |
| 81 | Et | t-Bu | H | H | — | CO₂CH₃ | * |
| 82 (Ex. 2) | Et | t-Bu | H | H | — | CO₂CH₂CH₂F | ** |
| 83 | Et | t-Bu | H | H | — | CO₂CH₂CF₃ | * |
| 84 | Et | t-Bu | H | H | — | CO₂CH₂C≡CH | * |
| 85 | Et | t-Bu | H | H | — | CO₂CH₂CH═CH₂ | * |
| 86 | Et | t-Bu | H | H | — | CO₂(CH₂)₃CH₃ | * |
| 87 | Et | t-Bu | H | H | 2-CH₃ | CO₂CH₃ | * |
| 88 | Et | t-Bu | H | H | 2-CH₃ | CO₂CH₂CH₃ | * |
| 89 | Et | t-Bu | H | H | 4-F | CO₂CH₂CH₃ | * |
| 90 | Et | t-Bu | H | H | 2-CH₃ | CO₂(CH₂)₂CH₃ | * |
| 91 | Et | t-Bu | H | H | 2-CH₃ | CO₂CH₂CH₂F | * |
| 92 | Et | t-Bu | H | H | 2-CH₃ | CO₂(i-Pr) | * |
| 93 | Et | t-Bu | H | H | 2-CH₃ | CO₂CH₂(c-Pr) | * |
| 94 | Et | t-Bu | H | H | 2-CH₃ | CO₂CH₂CH═CH₂ | * |
| 95 | Et | t-Bu | H | H | 4-OEt | CO₂CH₂CH₃ | * |
| 96 | Et | t-Bu | H | H | 4-OMe | CO₂CH₂CH₃ | * |
| 97 | Et | t-Bu | H | H | 2-OMe | CO₂CH₂CH₃ | * |
| 98 | Et | t-Bu | H | H | — | CO₂(t-Bu) | * |
| 99 | Et | t-Bu | H | H | 4-CH₃ | CO₂CH₂CH₃ | * |
| 100 | Et | t-Bu | H | H | 4-CH₃ | CO₂(i-Pr) | * |
| 101 | Et | t-Bu | H | H | 4-OMe | CO₂(i-Pr) | * |
| 102 | Me | t-Bu | H | H | — | C(═NOH)CH₃ | 189-191 |
| 103 | Me | CF₃ | H | H | — | C(═NOH)CH₃ | 189-191 |
| 104 | Me | t-Bu | H | H | 6-OMe | C(═NOH)CH₃ | 196-197 |
| 105 | Me | t-Bu | H | H | — | C(═NOCH₂CH₃)CH₃ | 94-95 |
| 106 | Me | t-Bu | H | H | — | C(═NOCH₃)CH₃ | 125-126 |
| 107 | Me | t-Bu | H | H | — | C(═NOH)CH₃ | * |
| 108 | Et | t-Bu | H | H | — | C(═NOH)CH₃ | 172-174 |
| 109 | Me | t-Bu | H | H | 4-F | C(═NOH)CH₃ | 165-167 |
| 110 | i-Pr | t-Bu | H | H | — | C(═NOH)CH₃ | * |
| 111 | Me | t-Bu | H | H | | 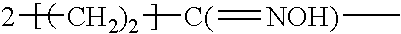 | * |
| 112 | CH₂CF₃ | t-Bu | H | H | — | C(═NOH)CH₃ | 145-147 |
| 113 | n-Pr | t-Bu | H | H | — | C(═NOH)CH₃ | 173-175 |
| 114 | CH═CH₂ | t-Bu | H | H | — | C(═NOH)CH₃ | * |
| 115 | CH₂CH₂F | t-Bu | H | H | — | C(═NOH)CH₃ | * |
| 116 | Me | t-Bu | H | H | | 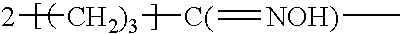 | * |
| 117 | CH₂CH═CH₂ | t-Bu | H | H | — | C(═NOH)CH₃ | * |
| 118 | Me | t-Bu | H | H | — | C(═NOH)CH₂CH₃ | 188-190 |
| 119 | n-Bu | t-Bu | H | H | — | C(═NOH)CH₃ | 161-163 |
| 120 | Me | s-Bu | H | H | — | C(═NOH)CH₃ | 139-141 |
| 121 | Me | t-Bu | H | H | — | CN | 95-99 |
| 122 | Et | t-Bu | H | H | — | C(O)CH₃ | 106-108 |
| 123 | Me | t-Bu | H | H | 4-F | C(O)CH₃ | 124-127 |
| 124 | Me | t-Bu | H | H | — | S(O)₂NH(i-Pr) | * |
| 125 | Me | t-Bu | H | H | — | S(O)₂NH(CH₂)₃CH₃ | * |
| 126 | CH₂CF₃ | t-Bu | H | H | — | C(O)CH₃ | 108-109 |
| 127 | CH═CH₂ | t-Bu | H | H | — | C(O)CH₃ | * |
| 128 | CH₂CH₂F | t-Bu | H | H | — | C(O)CH₃ | * |
| 129 | CH₂CH═CH₂ | t-Bu | H | H | — | C(O)CH₃ | * |
| 130 | Et | t-Bu | H | H | — | SCF₃ | * |
| 131 | Et | t-Bu | H | H | — | SCH₃ | * |
| 132 | CH₂CH₂F | t-Bu | H | H | — | SCF₃ | * |
| 133 | Et | t-Bu | H | H | — | S(O)₂CH₃ | * |
| 134 | Et | t-Bu | H | H | — | S(O)₂CF₃ | * |
| 135 | Et | t-Bu | H | H | — | S(O)CF₃ | * |
| 136 | Et | t-Bu | H | H | — | OCH₂CH₃ | * |
| 137 | Et | t-Bu | H | H | — | O(CH₂)₃CH₃ | * |
| 138 | Et | t-Bu | H | H | — | O(CH₂)₂CH₂F | * |
| 139 | Et | t-Bu | H | H | — | CN | * |
| 140 | Et | t-Bu | H | H | 6-F | CN | 153-154 |
| 141 | Et | t-Bu | H | C(O)Me | 4-Me | CO₂CH₂CH₃ | * |
| 236 (Ex. 18) | Et | t-Bu | H | H | 4-F | C(O)N(CH₃)₂ | ** |
| 242 | Et | t-Bu | H | H | 6-F | 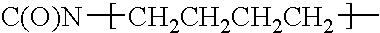 | * |
| 243 | Et | t-Bu | H | H | 6-F | 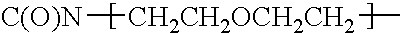 | * |
| 244 | Et | t-Bu | H | H | 6-F | C(O)N(CH₃)OCH₃ | * |
| 248 | Et | t-Bu | H | H | 4,6-di-F | CO₂(i-Pr) | * |
| 249 | Et | t-Bu | H | H | 4,6-di-F | CO₂CH₂CH₂CH₃ | * |
| 250 | Et | t-Bu | H | H | 4,6-di-F | CO₂CH₂CH₃ | * |
| 251 | Et | t-Bu | H | H | 4,6-di-F | C(O)NHCH₃ | * |
| 252 | Et | t-Bu | H | H | 4,6-di-F | C(O)NHCH₂CH₃ | * |
| 253 | Et | t-Bu | H | H | 4,6-di-F | C(O)N(CH₂CH₃)₂ | * |
| 254 | Et | t-Bu | H | H | 4,6-di-F | C(O)N(CH₃)CH₂CH₃ | * |
| 270 | Et | t-Bu | H | H | 4,6-di-F | C(O)N(CH₃)₂ | * |
| 271 | Et | t-Bu | H | H | 6-F | C(O)NH₂ | * |
| 272 | Et | t-Bu | H | H | — | OCF₃ | * |
| 273 | Et | t-Bu | H | H | — | OCH(CH₃)₂ | * |
| 275 | Et | t-Bu | F | H | 6-F | C(O)NHCH₂CH₃ | * |
| 276 (Ex. 21) | Et | t-Bu | F | H | 6-F | C(O)N(CH₃)₂ | ** |
| 285 | Et | t-Bu | H | H | — | C(O)N(CH₃)CH₂CH═CH₂ | * |
| 286 | Et | t-Bu | H | H | — | 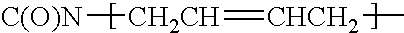 | * |
| 294 | Et | t-Bu | H | H | 6-F | C(S)N(CH₃)₂ | * |
| 295 | Et | 1-Me-c-Pr | H | H | — | CO₂CH₂CH₃ | * |
| 296 | Et | 1-Me-c-Pr | H | H | 6-F | CO₂CH₂CH₃ | * |
| 297 | Et | t-Bu | H | H | 6-OCH₃ | OCH₃ | * |
| 298 | Et | t-Bu | H | H | 4-OCH₃ | OCH₃ | * |
| 299 | Et | 1-Me-c-Pr | H | H | 6-F | C(O)N(CH₃)₂ | * |
| 300 | Et | 1-Me-c-Pr | H | H | 6-F | C(O)NHCH₂CH₃ | * |
| 301 | Et | 1-Me-c-Pr | H | H | 6-F | 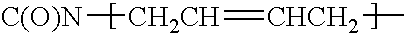 | * |
| 302 | Et | 1-Me-c-Pr | H | H | 6-F | C(O)NHCH₂C≡CH | * |
| 303 | Et | 1-Me-c-Pr | H | H | — | C(O)N(CH₃)₂ | * |
| 304 | Et | 1-Me-c-Pr | H | H | — | 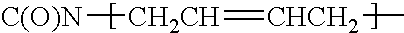 | * |
| 306 | Et | 1-Me-c-Pr | H | H | — | C(O)NHCH₂CH₃ | * |
| 311 | H | t-Bu | H | H | — | C(═NOH)CH₃ | * |
| 329 | Et | t-Bu | H | H | — | C(O)NHCH₂SCH₂CH₃ | * |
| 332 | Et | t-Bu | H | H | — | OCHF₂ | * |
| 333 | Et | t-Bu | H | H | — | OCF₂CHF₂ | * |
| 338 | Et | t-Bu | H | H | 6-F | OCF₃ | * |
| 347 | Et | t-Bu | H | H | 6-F | OCH₃ | * |
| 348 | Et | t-Bu | H | H | 6-F | OCH₂CH₃ | * |
| 349 | Et | t-Bu | H | H | 6-F | C(S)NHCH₂CH₃ | * |
| 350 | Et | t-Bu | H | H | 6-F | OCH₂CH₂F | * |
| 351 | H | t-Bu | H | H | — | C(O)NHCH₂CH₃ | * |
| 399 | Et | t-Bu | H | C(O)CH₃ | 6-F | C(O)NHCH₂CH₃ | * |
| 408 | Et | t-Bu | H | H | — | OS(O)₂CH₂CH₃ | * |
| 411 | Et | t-Bu | H | H | — | OCH₂C≡CH | * |
| 412 | Et | t-Bu | H | H | — | OCH₂CH═CH₂ | * |
| 413 | Et | t-Bu | H | H | 6-F | C(O)N(CH₂CH₃)C(O)CH₃ | * |
| 430 | Et | t-Bu | H | H | 6-F | C(O)NHCH₂CH═CH₂ | * |
| 431 | Et | t-Bu | H | H | 6-F | C(O)N(CH₂CH═CH₂)₂ | * |
| 442 | Et | t-Bu | H | H | 6-F | C(O)N(CH₂CH═CH₂)C(O)CH₃ | * |
| 443 | Et | t-Bu | H | C(O)Me | 6-F | C(O)NHCH₂CH═CH₂ | * |

* See Index Table L for ¹H NMR data.
** See synthesis example for ¹ H NMR data.

INDEX TABLE B

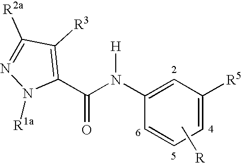

R can be one or more substituents; a dash (“-”) indicates no substituents.

| | | | | | | |
|---|---|---|---|---|---|---|
| Com- | | | | | | m.p. |
| pound | R^{1a} | R^{2a} | R³ | R | R⁵ | (° C.) |

| | | | | | |
|---|---|---|---|---|---|
| 352 | Et | 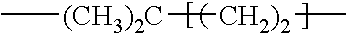 | 6-F | C(O)N(CH₃)₂ | ** |
| (Ex. 25) | | | | | |
| 353 | Et | 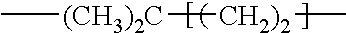 | 6-F | C(O)NHCH₂CH₃ | * |

* See Index Table L for ¹H NMR data.
** See synthesis example for ¹H NMR data.

INDEX TABLE C

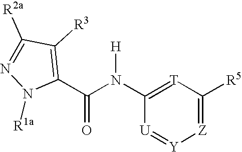

| Compound | R^{1a} | R^{2a} | R³ | T | U | Y | Z | R⁵ | m.p. (° C.) |
|---|---|---|---|---|---|---|---|---|---|
| 142 | Et | t-Bu | H | CH | N | CH | CH | 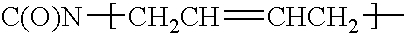 | * |
| 143 (Ex. 8) | Et | t-Bu | H | N | CH | CH | CH | CO₂CH₃ | ** |
| 144 | Et | t-Bu | H | N | CH | CH | CH | C(O)NHCH₂CH₃ | * |
| 145 | Et | t-Bu | H | N | CH | CH | CH | C(O)N(CH₂CH₃)₂ | * |
| 146 | Et | t-Bu | H | N | CH | CH | CH | C(O)NH(c-Pr) | * |
| 147 | Et | t-Bu | H | N | CH | CH | CH | C(O)NHCH₂C≡CH | * |
| 148 | Et | t-Bu | H | CH | N | CCH₃ | CH | CO₂CH₃ | * |
| 149 | Et | t-Bu | H | CH | N | CCH₃ | CH | C(O)NHCH₂CH₃ | * |
| 150 | Et | t-Bu | H | N | CH | CCH₃ | CH | C(O)NHCH₂CH₃ | * |
| 151 (Ex. 10) | Et | t-Bu | H | CH | N | CH | CH | CO₂CH₃ | ** |
| 152 | Et | t-Bu | H | CH | N | CH | CH | C(O)NHCH₂CH₃ | * |
| 153 | Et | t-Bu | H | CH | N | CH | CH | C(O)N(CH₂CH₃)₂ | * |
| 154 | Et | t-Bu | H | CH | N | CH | CH | C(O)NH(l-Bu) | * |
| 155 | Et | t-Bu | H | CH | N | CH | CH | C(O)NHCH₂(c-Pr) | * |
| 156 (Ex. 11) | Et | t-Bu | H | CH | N | CH | CH | C(O)N(CH₃)₂ | ** |
| 157 | Et | t-Bu | H | CH | N | CH | CH | CO₂CH₂CH₃ | * |
| 158 | Et | t-Bu | H | N | CH | CH | CH | C(O)N(CH₃)CH₂CN | * |
| 159 | Et | t-Bu | H | CH | N | CH | CH | C(O)N(CH₃)CH₂CH═CH₂ | * |
| 160 | Et | t-Bu | H | CH | N | CH | CH | C(O)N(CH₃)CH₂C≡CH | * |
| 161 | Et | t-Bu | H | CH | N | CH | CH | C(O)NH(CH₂)₂OCH₃ | * |
| 162 (Ex. 9) | Et | t-Bu | H | N | CH | CH | CH | C(O)N(CH₃)₂ | ** |
| 163 | Et | t-Bu | H | N | CH | CH | CH | C(O)N(CH₃)CH₂CH═CH₂ | * |
| 164 | Me | t-Bu | H | N | N | CH | CH | C(═NOH)CH₃ | 196-197 |
| 165 | Et | t-Bu | H | N | N | CH | CH | OCH₃ | * |
| 166 | Et | t-Bu | H | N | N | COCH₃ | CH | OCH₃ | * |
| 167 | Et | t-Bu | H | N | N | COCH₃ | CH | SCH₂F | * |
| 245 | Et | t-Bu | H | N | CH | CH | CH | 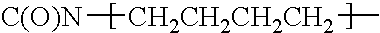 | * |
| 246 | Et | t-Bu | H | N | CH | CH | CH | C(O)N(CH₃)OCH₃ | * |
| 247 | Et | t-Bu | H | N | CH | CH | CH | 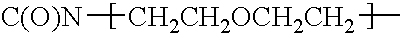 | * |
| 255 (Ex. 19) | Et | t-Bu | H | CH | CH | N | CH | CO₂CH₂CH₃ | ** |
| 256 | Et | t-Bu | H | CH | CH | CH | N | C(O)N(CH₃)₂ | * |
| 257 | Et | t-Bu | H | CH | CH | CH | N | C(O)NHCH₂CH₃ | * |
| 258 | Et | t-Bu | H | CH | CH | N | CH | C(O)N(CH₃)₂ | * |
| 259 | Et | t-Bu | H | CH | CH | N | CH | C(O)NHCH₂CH₃ | * |
| 260 | Et | t-Bu | H | CH | CH | N | CH | 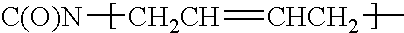 | * |
| 261 (Ex. 20) | Et | t-Bu | H | CH | CH | N | CH | C(O)N(CH₂CH₃)₂ | ** |
| 262 | Et | t-Bu | H | CH | CH | N | CH | C(O)N(CH₂CH═CH₂)₂ | * |
| 263 | Et | t-Bu | H | CH | CH | N | CH | C(O)N(CH₃)CH₂CH₃ | * |
| 264 | Et | t-Bu | H | CH | CH | N | CH | C(O)NHCH₂C≡CH | * |
| 265 | Et | t-Bu | H | N | COCH₃ | CH | CH | CO₂CH₃ | * |
| 266 | Et | t-Bu | H | N | COCH₃ | CH | CH | C(O)NHCH₂CH₃ | * |
| 274 | Et | t-Bu | F | N | CH | CH | CH | C(O)N(CH₃)₂ | * |
| 277 | Et | t-Bu | H | N | CF | CH | CH | C(O)N(CH₃)₂ | * |
| 278 | Et | t-Bu | H | N | CF | CH | CH | C(O)NHCH₂CH₃ | * |
| 279 | Et | t-Bu | H | N | CF | CH | CH | C(O)NHCH₂C≡CH | * |
| 280 | Et | t-Bu | H | N | CF | CH | CH | C(O)N(CH₃)CH₂CH₃ | * |
| 281 | Et | t-Bu | H | N | CF | CH | CH | 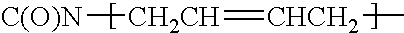 | * |
| 282 | Et | t-Bu | H | CH | CH | CH | N | 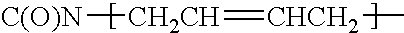 | * |
| 283 | Et | t-Bu | H | CH | CH | CH | N | C(O)NHCH₂C≡CH | * |
| 284 | Et | t-Bu | H | CH | CH | CH | N | C(O)NHCH₂CH₂F | * |
| 287 | Et | t-Bu | H | N | CH | N | CH | C(O)N(CH₃)₂ | * |
| 288 | Et | t-Bu | H | N | CH | N | CH | C(O)NHCH₂CH₃ | * |
| 289 | Et | t-Bu | H | N | CH | N | CH | C(O)NHCH₂C≡CH | * |
| 290 | Et | t-Bu | H | N | CH | N | CH | 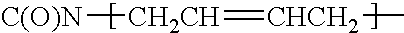 | * |
| 291 | Et | t-Bu | H | N | CH | N | CH | C(O)NHCH₂CF₃ | * |
| 312 | Me | t-Bu | H | N | CH | CH | CH | CO₂CH₃ | * |
| 313 | Me | t-Bu | H | N | CH | CH | CH | CO₂CH₂CH₃ | * |
| 314 | Me | t-Bu | H | N | CH | CH | CH | C(O)N(CH₂CH₃)₂ | * |
| 315 | Me | t-Bu | H | N | CH | CH | CH | C(O)NH(c-Pr) | * |
| 316 | Me | t-Bu | H | N | CH | CH | CH | C(O)NHCH₂C≡CH | * |
| 317 | Me | t-Bu | H | N | CH | CH | CH | CO₂CH₂C≡CH | * |
| 318 | Me | t-Bu | H | N | CH | CH | CH | C(O)NHCH₂CH═CH₂ | * |
| 334 | Et | t-Bu | H | CH | N | CH | CH | C(S)N(CH₃)₂ | 170-172 |

* See Index Table L for ¹H NMR data.
** See synthesis example for ¹H NMR data.

INDEX TABLE D

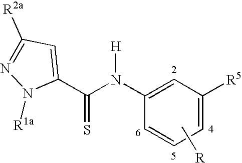

R can be one or more substituents; a dash (“-”) indicates no substituents.

| Compound | R^{1a} | R^{2a} | R | R⁵ | m.p. (° C.) |
|---|---|---|---|---|---|
| 305 | Et | t-Bu | 6-F | C(S)N(CH₃)₂ | 90-92 |
| 346 | Et | t-Bu | 6-F | CO₂CH₂CH₃ | * |

* See Index Table L for ¹H NMR data.

INDEX TABLE E

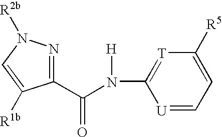

| Compound | R^{1b} | R^{2b} | T | U | R⁵ | m.p.(° C.) |
|---|---|---|---|---|---|---|
| 168 (Ex. 12) | Et | t-Bu | CH | CH | C(O)N(CH₂CH₃)₂ | ** |
| 169 | CF₃ | t-Bu | CH | CH | C(O)N(CH₂CH₃)₂ | * |
| 170 | CF₃ | t-Bu | CH | CH | C(O)NHCH₂CH₃ | * |
| 171 | CF₃ | t-Bu | CH | CH | C(═NOH)CH₃ | * |
| 218 | Cl | t-Bu | CH | CH | C(O)N(CH₂CH₃)₂ | * |
| 219 | Cl | t-Bu | CH | CH | C(O)NHCH₂CH₃ | * |
| 220 | Br | t-Bu | CH | CH | C(O)N(CH₂CH₃)₂ | * |
| 221 (Ex. 14) | Br | t-Bu | CH | CH | C(O)NHCH₂CH₃ | ** |
| 232 | Et | t-Bu | CH | N | C(O)N(CH₃)₂ | 163-165 |
| 233 | Et | t-Bu | CH | N | C(O)NHCH₂CH₃ | 141-143 |
| 234 | Et | t-Bu | CH | N | C(O)N(CH₂CH₃)₂ | 90-91 |
| 235 | Et | t-Bu | CH | N | C(O)N(CH₃)CH₂CH₃ | 103-105 |
| 330 | Et | t-Bu | N | CH | C(O)N(CH₃)₂ | 159-160 |

* See Index Table L for ¹H NMR data.
** See synthesis example for ¹H NMR data.

INDEX TABLE F

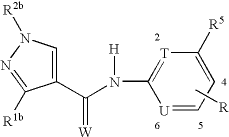

R can be one or more substituents; a dash (“—”) indicates no substituents.

| Compound | R^{1b} | R^{2b} | W | T | U | R | R⁵ | m.p.(° C.) |
|---|---|---|---|---|---|---|---|---|
| 172 | CF₃ | Et | O | CH | CH | — | C(O)NHCH₂CH₃ | * |
| 179 | Me | i-Pr | O | CH | CH | — | C(O)CH₃ | 116-118 |
| 180 | Me | i-Pr | O | CH | CH | — | C(═NOH)CH₃ | 192-193 |
| 181 | Et | t-Bu | O | CH | CH | — | C(O)NHCH₂CH₃ | 160-163 |
| 182 (Ex. 13) | Et | t-Bu | O | CH | CH | — | C(O)N(CH₂CH₃)₂ | ** |
| 183 | Me | t-Bu | O | CH | CH | — | C(O)NHCH₂CH₃ | * |
| 184 | Me | t-Bu | O | CH | CH | — | C(O)N(CH₂CH₃)₂ | * |
| 185 | n-Pr | t-Bu | O | CH | CH | — | C(O)N(CH₂CH₃)₂ | * |
| 186 | Et | t-Bu | O | CH | CH | — | CO₂CH₂CH₃ | * |
| 187 | Et | t-Bu | O | CH | CH | — | C(O)CH₃ | * |
| 188 | Et | t-Bu | O | CH | CH | — | C(═NOH)CH₃ | * |
| 189 | Me | t-Bu | O | CH | CH | — | O(iPr) | * |
| 190 | Me | t-Bu | O | CH | CH | — | OCH₂CH₃ | * |
| 191 | CF₃ | t-Bu | O | CH | CH | — | C(O)N(CH₂CH₃)₂ | * |
| 192 | CF₃ | t-Bu | O | CH | CH | — | C(O)NHCH₂CH₃ | * |
| 193 | Et | t-Bu | O | CH | CF | — | C(O)NHCH₂CH₃ | * |
| 194 | Et | t-Bu | O | CH | CF | — | C(O)NHCH₃ | * |
| 222 (Ex.16) | Et | t-Bu | O | CH | CF | — | C(O)N(CH₃)₂ | ** |
| 223 | Et | t-Bu | O | CH | CF | — | C(O)N(CH₂CH₃)₂ | 179-180 |
| 224 | Et | t-Bu | O | CH | CF | — | C(O)N(CH₃)CH₂CH₃ | 138-139 |
| 238 | Et | t-Bu | O | CH | N | — | CO₂CH₂CH₃ | * |
| 239 | Et | t-Bu | O | CH | CH | 4-F | C(O)N(CH₃)₂ | 170-171 |
| 240 | Et | t-Bu | O | CH | CH | 4-F | C(O)N(CH₂CH₃)₂ | 163-165 |
| 241 (Ex.17) | Et | t-Bu | O | CH | CH | 4-F | C(O)NHCH₂CH₃ | ** |
| 292 | Et | t-Bu | O | N | CH | — | C(O)N(CH₃)₂ | 148-149 |
| 293 | Et | t-Bu | O | CH | N | — | C(O)N(CH₃)₂ | 150-151 |
| 344 | Me | t-Bu | O | CH | CH | 6-F | C(O)N(CH₃)₂ | 201-202 |
| 345 | Me | t-Bu | O | CH | CH | 6-F | C(O)NHCH₂CH₃ | 203-204 |
| 452 | Et | t-Bu | O | CH | N | — | C(O)NHCH₂CH₃ | * |
| 453 | Et | t-Bu | S | CH | CH | 6-F | C(S)N(CH₃)₂ | 183-184 |
| 454 | Et | t-Bu | O | CH | CH | 6-F | C(S)N(CH₃)₂ | 162-164 |

* See Index Table L for ¹H NMR data.
** See synthesis example for ¹H NMR data.

INDEX TABLE G

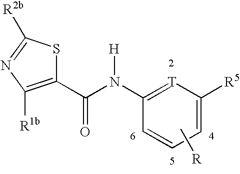

R can be one or more substituents; a dash (“—”) indicates no substituents.

| | | | | | | m.p. |
|---|---|---|---|---|---|---|
| Compound | R^{1b} | R^{2a} | T | R | R⁵ | (° C.) |
| 195 | Me | t-Bu | CH | — | C(═NOH)CH₃ | * |
| 196 | Me | t-Bu | CH | — | C(O)CH₃ | * |
| 197 | Me | t-Bu | CH | — | CO₂CH₃ | * |
| 198 | Me | t-Bu | CH | — | C(O)NHCH₂CH₃ | * |
| 199 | Me | t-Bu | CH | — | C(O)N(CH₂CH₃)₂ | * |
| 200 | Me | t-Bu | CH | — | C(O)NHCH₂CH₂F | * |
| 201 | Me | t-Bu | CH | — | C(O)NHCH₂CF₃ | * |
| 202 | Me | t-Bu | CH | — | C(O)NHCH₂CH═CH₂ | * |
| 203 | Me | t-Bu | CH | — | C(O)NHCH₂C≡CH | * |
| 204 | Et | t-Bu | CH | — | CO₂CH₂CH₃ | * |
| 205 | Et | t-Bu | CH | — | C(O)N(CH₂CH₃)₂ | * |
| 206 | Et | t-Bu | CH | — | C(O)NHCH₂CF₃ | * |
| 207 | Et | t-Bu | CH | — | C(O)NHCH₂CH═CH₂ | * |
| 209 | Et | t-Bu | CH | — | C(O)NHCH₂CH₂F | * |
| 210 | Et | t-Bu | CH | — | C(O)NHCH₂CH₃ | * |
| 211 | Et | t-Bu | CH | — | C(O)NH(I-Bu) | * |
| 212 | Et | t-Bu | CH | — | C(O)NHCH₂(c-Pr) | * |
| 213 | Et | t-Bu | N | — | C(O)NHCH₂CH₃ | * |
| 214 | Et | t-Bu | N | — | C(O)N(CH₂CH₃)₂ | * |
| 215 | Et | t-Bu | N | — | C(O)NHCH₂(c-Pr) | * |
| 216 | Et | t-Bu | N | — | C(O)NH(t-Bu) | * |
| 217 | Et | t-Bu | N | — | C(O)NHCH₂C≡CH | * |
| 225 | Et | t-Bu | CH | 6-F | C(O)N(CH₃)₂ | * |
| 226 | Et | t-Bu | CH | 6-F | C(O)NHCH₂CH₃ | * |
| 227 | Me | t-Bu | CH | — | C(O)N(CH₃)CH₂CH₃ | * |
| 228 | Et | t-Bu | CH | — | C(O)N(CH₃)₂ | * |
| 229 | Et | t-Bu | CH | — | C(O)N(CH₃)CH₂CH₃ | * |
| 230 | Et | t-Bu | CH | 6-F | C(O)N(CH₂CH₃)₂ | * |
| 231 | Et | t-Bu | CH | 6-F | C(O)N(CH₃)CH₂CH₃ | * |
| 237 | t-Bu | Et | CH | 6-F | C(O)NHCH₂CH═CH₂ | * |
| 319 | Et | t-Bu | CH | — | C(O)NHCH₂C≡CH | * |
| 326 | Me | t-Bu | CH | — | C(O)N(CH₃)₂ | * |
| 327 | t-Bu | Et | CH | 6-F | C(O)N(CH₂CH═CH₂)₂ | * |

* See Index Table L for ¹H NMR data.

INDEX TABLE H

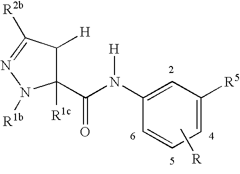

R can be one or more substituents; a dash (“—”) indicates no substituents.

| Com- | | | | | | m.p. |
|---|---|---|---|---|---|---|
| pound | R^{1a} | R^{2a} | R^{1c} | R | R⁵ | (° C.) |
| 267 | Et | t-Bu | H | 6-F | CO₂CH₂CH₃ | ** |
| (Ex. 22) | | | | | | |
| 268 | Et | t-Bu | H | 6-F | C(O)N(CH₃)₂ | ** |
| (Ex. 23) | | | | | | |
| 269 | Et | t-Bu | H | 6-F | C(O)NHCH₂CH₃ | * |
| 307 | Et | t-Bu | H | 6-F | C(O)N(CH₃)CH₂CH₃ | * |
| 308 | Et | t-Bu | H | 6-F | C(O)N(CH₂CH₃)₂ | * |
| 309 | Et | t-Bu | H | 6-F | C(O)N [symbol] CH₂CH₂CH₂CH₂ [symbol] | * |
| 310 | Et | t-Bu | H | 6-F | C(O)N [symbol] CH₂CH═CHCH₂ [symbol] | * |
| 331 | Et | t-Bu | H | 6-F | C(O)N [symbol] CH₂CH₂CH₂CH₂CH₂ [symbol] | * |

* See Index Table L for ¹H NMR data.
** See synthesis example for ¹H NMR data.

INDEX TABLE I

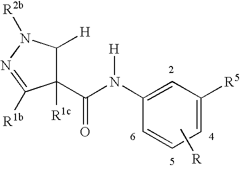

R can be one or more substituents; a dash (“—”) indicates no substituents.

| Com- | | | | | | m.p. |
|---|---|---|---|---|---|---|
| pound | R^{1b} | R^{2b} | R^{1c} | R | R⁵ | (° C.) |
| 335 | Et | t-Bu | H | 6-F | CO₂CH₂CH₃ | * |
| 336 | Et | t-Bu | H | 6-F | C(O)NHCH₂CH₃ | ** |
| (Ex. 24) | | | | | | |
| 340 | Et | t-Bu | H | 6-F | C(O)N [symbol] CH₂CH═CHCH₂ [symbol] | * |
| 341 | Et | t-Bu | H | 6-F | C(O)N [symbol] CH₂CH₂CH₂CH₂ [symbol] | * |
| 342 | Et | t-Bu | H | 6-F | C(O)N(CH₃)CH₂CH₃ | * |
| 343 | Et | t-Bu | H | 6-F | C(O)N(CH₃)₂ | * |

* See Index Table L for ¹H NMR data.
** See synthesis example for ¹H NMR data.

INDEX TABLE J

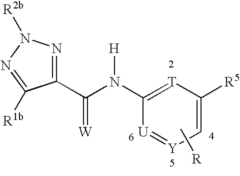

R can be one or more substituents; a dash (“—”) indicates no substituents.

| Compound | R^{1b} | R^{2b} | W | T | U | Y | R | R⁵ | m.p.(° C.) |
|---|---|---|---|---|---|---|---|---|---|
| 354 | Et | c-pentyl | O | CH | CH | CH | — | C(O)N [symbol] CH₂CH═CHCH₂ [symbol] | * |
| 355 | Et | c-pentyl | O | CH | CH | CH | — | C(O)N(CH₃)₂ | * |
| 356 | Et | c-pentyl | O | CH | CH | CH | — | C(O)NHCH₂CH₃ | * |
| 357 | Et | i-Pr | O | CH | CH | CH | — | C(O)NHCH₂CH₃ | * |
| 358 (Ex. 27) | Et | i-Pr | O | CH | CH | CH | — | C(O)N(CH₃)₂ | ** |
| 359 | Et | i-Pr | O | CH | CH | CH | — | C(O)N [symbol] CH₂CH═CHCH₂ [symbol] | * |
| 360 (Ex. 28) | Et | t-Bu | O | CH | CH | CH | — | CO₂CH₂CH₃ | ** |
| 361 | Et | t-Bu | O | CH | CH | CH | 6-F | C(O)N(CH₃)₂ | * |
| 362 | Et | t-Bu | O | CH | CH | CH | 6-F | C(O)NCH₂CH₃ | * |
| 363 | Et | t-Bu | O | CH | CH | CH | 6-F | C(O)N [symbol] CH₂CH═CHCH₂ [symbol] | * |
| 364 | Et | t-Bu | O | CH | CH | CH | — | C(O)N(CH₃)₂ | * |
| 365 (Ex. 29) | Et | t-Bu | O | CH | CH | CH | — | C(O)NCH₂CH₃ | ** |
| 366 | Et | t-Bu | O | CH | CH | CH | — | C(O)N [symbol] CH₂CH═CHCH₂ [symbol] | * |
| 367 (Ex. 26) | Et | i-Pr | O | CH | CH | CH | — | CO₂CH₂CH₃ | ** |
| 368 | Et | C(CH₃)₂CH₂CH₃ | O | CH | CH | CH | 6-F | C(O)N(CH₃)₂ | * |
| 369 | Et | C(CH₃)₂CH₂CH₃ | O | CH | CH | CH | — | C(O)N [symbol] CH₂CH═CHCH₂ [symbol] | * |
| 370 | Et | C(CH₃)₂CH₂CH₃ | O | CH | CH | CH | 6-F | C(O)NHCH₂CH₃ | * |
| 371 | Et | C(CH₃)₂CH₂CH₃ | O | CH | CH | CH | 6-F | C(O)N [symbol] CH₂CH═CHCH₂ [symbol] | * |
| 372 | Et | C(CH₃)₂CH₂CH₃ | O | CH | CH | CH | — | C(O)N(CH₃)₂ | * |
| 373 | Et | C(CH₃)₂CH₂CH₃ | O | CH | CH | CH | 6-F | C(O)OCH₂CH₃ | * |
| 374 | Et | C(CH₃)₂CH₂CH₃ | O | CH | CH | CH | — | OCF₃ | * |
| 375 | Et | C(CH₃)₂CH₂CH₃ | O | CH | CH | CH | — | C(O)OCH₂CH₃ | * |
| 376 | Et | C(CH₃)₂CH₂CH₃ | O | CH | CH | CH | — | C(O)NHCH₂CH₃ | * |
| 377 | Me | t-Bu | O | CH | CH | CH | 6-F | C(O)NHCH₂CH₃ | * |
| 378 | Me | t-Bu | O | CH | CH | CH | 6-F | C(O)N(CH₃)₂ | * |
| 379 | Et | i-Pr | O | CH | CH | CH | — | C(O)NHCH₂C≡CH | * |
| 380 | Et | t-Bu | O | CH | CH | CH | — | C(O)NHCH₂C≡CH | * |
| 381 | Et | t-Bu | O | CH | CH | CH | 6-F | C(O)N [symbol] CH₂CH₂CH₂CH₂CH₂ [symbol] | * |
| 382 | Et | i-Pr | O | CH | CH | CH | — | C(O)NHCH₂CH═CH₂ | * |
| 383 | Et | t-Bu | O | CH | CH | CH | 6-F | C(O)NHCH₂CH═CH₂ | * |
| 384 | Et | t-Bu | O | CH | CH | CH | — | C(O)N(CH₃)CH₂CH₃ | * |
| 385 | Et | i-Pr | O | CH | CH | CH | — | C(O)N [symbol] CH₂CH₂CH₂CH₂CH₂ [symbol] | * |
| 386 | Et | i-Pr | O | CH | CH | CH | — | C(O)N(CH₃)CH₂CH₃ | * |
| 387 | Et | t-Bu | O | CH | CH | CH | 6-F | C(O)N(CH₂CH═CH₂)₂ | * |
| 388 | Et | r-Bu | O | CH | CH | CH | 6-F | C(O)N(CH₃)CH₂CH₃ | * |
| 389 | Et | t-Bu | O | CH | CH | CH | 6-F | C(O)NHCH₂C≡CH | * |
| 390 | Et | i-Pr | O | CH | CH | CH | — | C(O)N(CH₂CH═CH₂)₂ | * |
| 391 | Me | t-Bu | O | CH | CH | CH | — | C(O)N(CH₃)₂ | * |
| 392 | Me | t-Bu | O | CH | CH | CH | — | C(O)NHCH₂CH₃ | * |
| 393 | Me | t-Bu | O | CH | CH | CH | — | C(O)N [symbol] CH₂CH═CHCH₂ [symbol] | * |
| 394 | Me | t-Bu | O | CH | CH | CH | — | C(O)NHCH₂C≡CH | * |
| 395 | Et | t-Bu | O | CH | CH | CH | — | C(S)N(CH₃)₂ | * |
| 396 | Et | t-Bu | S | CH | CH | CH | — | C(S)N(CH₃)₂ | * |
| 397 | Me | i-Pr | O | CH | CH | CH | — | C(O)OCH₂CH₃ | * |
| 398 | Me | i-Pr | O | CH | CH | CH | 6-F | C(O)OCH₃ | * |
| 400 | Me | i-Pr | O | CH | CH | CH | 6-F | C(O)N(CH₃)₂ | * |
| 401 | Me | i-Pr | O | CH | CH | CH | 6-F | C(O)NHCH₂CH₃ | * |
| 402 | Me | i-Pr | O | CH | CH | CH | 6-F | C(O)N [symbol] CH₂CH═CHCH₂ [symbol] | * |
| 403 | Me | i-Pr | O | CH | CH | CH | 6-F | C(O)N(CH₃)CH₂CH₃ | * |
| 404 | Me | i-Pr | O | CH | CH | CH | — | C(O)N(CH₃)₂ | * |
| 405 | Me | i-Pr | O | CH | CH | CH | — | C(O)NHCH₂CH₃ | * |
| 406 | Me | i-Pr | O | CH | CH | CH | — | C(O)N [symbol] CH₂CH═CHCH₂ [symbol] | * |
| 407 | Me | i-Pr | O | CH | CH | CH | — | C(O)N(CH₃)CH₂CH₃ | * |
| 409 | Et | i-Pr | O | CH | CH | CH | — | C(S)N(CH₃)₂ | * |
| 410 | Et | i-Pr | S | CH | CH | CH | — | C(S)(CH₃)₂ | * |
| 414 | Et | t-Bu | O | CH | CH | CH | — | C(O)NHCH₂CH═CH₂ | * |
| 415 | Et | t-Bu | O | CH | CH | CH | — | C(O)N(CH₂CH═CH₂)₂ | * |
| 416 | Et | t-Bu | O | CH | CH | CH | — | C(O)N [symbol] CH₂CH₂CH₂CH₂CH₂ [symbol] | * |
| 417 | Et | t-Bu | O | CH | CH | CH | — | C(O)NH(c-Pr) | * |
| 418 | Me | t-Bu | O | CH | CH | CH | — | C(O)N(CH₂CH₃)₂ | * |
| 419 | Me | t-Bu | O | CH | CH | CH | — | C(S)N(CH₃)₂ | * |
| 420 | Me | t-Bu | O | CH | CH | CH | — | C(S)NHCH₂CH₃ | * |
| 421 | Me | t-Bu | O | CH | N | CH | — | C(O)OCH₂CH₃ | * |
| 422 | Me | t-Bu | O | CH | CH | N | — | C(O)OCH₂CH₃ | * |
| 423 | Me | t-Bu | O | CH | N | CH | — | C(O)N(CH₃)₂ | * |
| 424 | Me | t-Bu | O | CH | N | CH | — | C(O)NHCH₂CH₃ | * |
| 425 | Me | t-Bu | O | CH | N | CH | — | C(O)N(CH₂CH₃)₂ | * |
| 426 | Me | t-Bu | O | CH | CH | N | — | C(O)N(CH₃)₂ | * |
| 427 | Me | t-Bu | O | CH | CH | N | — | C(O)NHCH₂CH₃ | * |
| 428 | Me | t-Bu | O | CH | CH | N | — | C(O)N(CH₂CH₃)₂ | * |
| 429 | Br | t-Bu | O | CH | CH | CH | — | C(O)N(CH₃)₂ | * |
| 432 | Br | t-Bu | O | CH | CH | CH | — | C(O)OCH₂CH₃ | * |
| 433 | Br | t-Bu | O | CH | CH | CH | — | C(O)N [symbol] CH₂CH═CHCH₂ [symbol] | * |
| 434 | Br | t-Bu | O | CH | CH | CH | — | C(O)NHCH₂CH₃ | * |
| 435 | Br | t-Bu | O | CH | CH | CH | — | C(O)NH(c-Pr) | * |
| 436 | Me | t-Bu | O | N | CH | CH | — | C(O)N(CH₃)₂ | * |
| 437 | Me | t-Bu | O | N | CH | CH | — | C(O)NHCH₂CH₃ | * |
| 438 | Me | t-Bu | O | N | CH | CH | — | C(O)NHCH₂CH═CH₂ | * |
| 439 | Me | t-Bu | O | N | CH | CH | — | C(O)N(CH₂CH═CH₂)₂ | * |
| 440 | Me | t-Bu | O | N | CH | CH | — | C(O)N(CH₂CH₃)₂ | * |
| 441 | Me | t-Bu | O | N | CH | CH | — | C(O)N [symbol] CH₂CH═CHCH₂ [symbol] | * |
| 444 | Me | t-Bu | O | N | CH | CH | — | C(O)N(CH₃)CH₂CH═CHCH₂ | * |
| 445 | CH═CH₂ | t-Bu | O | CH | CH | CH | — | C(O)N(CH₃)₂ | * |

* See Index Table L ¹H NMR data.
** See synthesis example ¹H NMR data.

INDEX TABLE K

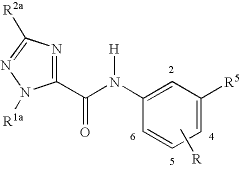

R can be one or more substituents; a dash (“—”) indicates no substituents.

| Compound | R^{1a} | R^{2a} | R | R⁵ | m.p.(° C.) |
|---|---|---|---|---|---|
| 446 (Ex. 30) | Et | t-Bu | — | C(O)NHCH₂CH₃ | ** |
| 447 | Et | t-Bu | 6-F | C(O)NHCH₂CH₃ | * |
| 448 | Et | t-Bu | — | C(O)N(CH₃)₂ | * |
| 449 | Et | t-Bu | — | OCF₃ | * |
| 450 | Et | t-Bu | 6-F | C(O)N(CH₃)₂ | * |
| 451 | Et | t-Bu | 6-F | C(O)OCH₂CH₃ | * |

* See Index Table L for ¹H NMR data.
** See synthesis example for ¹H NMR data.

INDEX TABLE L

| Compound | ¹H NMR Data (CDCl₃ solution unless indicated otherwise)^{a} |
|---|---|
| 1 | δ 8.56(dd, 1H), 8.00(br s, 1H, NH), 7.24(m, 2H), 6.54 |
| | (s, 1H), 5.92(m, 1H), 5.82(m, 1H), 4.57(q, 2H), 4.42(m, |
| | 2H), 4.20(m, 2H), 1.42(t, 3H), 1.28(s, 9H). |
| 3 | δ 8.82(m, 1H), 7.92(br s, 1H, NH), 7.62(m, 1H), 7.24(s, |
| | 1H), 6.56(br s, 1H, NH), 6.42(s, 1H), 4.56(m, 4H), 3.78 |
| | (m, 2H), 1.46(t, 3H), 1.33(s, 9H). |
| 4 | δ 8.42(m, 1H), 7.92(br s, 1H, NH), 7.24(m, 2H), 6.42(s, |
| | 1H), 4.52(q, 2H), 3.42(m, 4H), 1.46(m, 6H), 1.30(s, |
| | 9H). |
| 5 | δ 8.80(m, 1H), 7.92(br s, 1H, NH), 7.64(m, 1H), 7.24 |
| | (m, 1H), 6.56(t, 1H, NH), 6.51(s, 1H), 4.56(q, 2H), 4.06 |
| | (m, 2H), 1.46(t, 3H), 1.33(s, 9H). |
| 8 | δ 8.20(m, 1H), 8.12(s, 1H), 8.08(br s, 1H), 6.57(s, 1H), |
| | 6.30(s, 1H), 3.50(m, 2H), 1.51(t, 3H), 1.43(t, 3H), 1.33 |
| | (s, 9H), 1.25(t, 3H). |
| 9 | δ 8.40(dd, 1H), 8.14(br s, 1H, NH), 7.22(m, 2H), 6.56 |
| | (s, 1H), 5.86(m, 1H), 5.28(m, 2H), 4.58(q, 2H), 3.08(s, |
| | 3H), 1.44(t, 3H), 1.34(s, 9H). |
| 10 | δ 8.52(dd, 1H), 7.92(br s, 1H, NH), 7.22(m, 2H), 6.58 |
| | (s, 1H), 4.60(q, 2H), 4.02(m, 2H), 3.20(s, 3H), 2.40(s, |
| | 1H), 1.44(t, 3H), 1.34(s, 9H). |
| 11 | δ 8.68(dd, 1H), 7.84(br s, 1H, NH), 7.62(m, 1H), 7.24 |
| | (m, 1H), 6.60(s, 1H, NH), 6.49(s, 1H), 4.60(q, 2H), 3.62 |
| | (m, 4H), 3.39(s, 3H), 1.44(t, 3H), 1.34(s, 9H). |
| 12 | δ 8.40 (dd, 1H), 8.00 (br s, 1H, NH), 7.24 (m, 2H), 6.56 |
| | (s, 1H), 4.60 (q, 2H), 3.42 (m, 2H), 3.00 (s, 3H), 1.44 (t, |
| | 3H), 1.34 (m, 1H), 1.28 (s, 9H), 1.00 (d, 6H). |
| 13 | δ 8.62 (dd, 1H), 7.92 (br s, 1H, NH), 7.66 (m, 1H), 7.24 |
| | (m, 1H), 6.48 (s, 1H), 6.32 (br s, 1H, NH), 4.56 (q, 4H), |
| | 3.48 (q, 2H), 3.00 (m, 1H), 1.31 (t, 3H), 1.28 (t, 3H), 1.24 |
| | (d, 6H). |
| 14 | δ 8.40 (dd, 1H), 8.00 (br s, 1H, NH), 7.18 (m, 2H), 6.51 (s, |
| | 1H), 4.56 (q, 2H), 3.10 (s, 3H), 3.08 (m, 1H), 3.03 (s, 3H), |
| | 1.41 (t, 3H), 1.24 (d, 6H). |
| 15 | δ 9.00 (s, 1H, NH), 8.60 (dd, 1H), 7.12 (m, 2H), 6.55 (s, |
| | 1H), 4.16 (q, 2H), 3.42 (m, 4H), 3.03 (m, 1H), 1.41 (t, 3H), |
| | 1.24 (d, 6H). |
| 16 | δ 8.40 (br s, 1H), 8.20 (m, 1H), 7.20 (dd, 2H), 6.52 (s, |
| | 1H), 4.57 (q, 2H), 3.40 (m, 2H), 3.00 (s, 3H), 1.44 (t, 6H), |
| | 1.34 (s, 9H). |
| 17 | δ 8.38 (dd, 1H), 8.02 (br s, 1H, NH), 7.12 (m, 2H), 6.60 (s, |
| | 1H), 4.57 (q, 2H), 3.60 (t, 2H), 3.20 (t, 3H), 3.00 (s, 3H), |
| | 1.60 (m, 2H) 1.44 (t, 6H), 1.34 (s, 9H). |
| 18 | δ 8.40 (dd, 1H), 8.04 (br s, 1H, NH), 7.18 (m, 2H), 6.41 (s, |
| | 1H), 4.56 (q, 2H), 3.10 (s, 3H), 3.08 (m, 1H), 3.03 (s, 3H), |
| | 1.96 (m, 1H) 1.34 (t, 3H), 0.94 (m, 4H). |
| 19 | δ 8.34 (dd, 1H), 8.06 (br s, 1H, NH), 7.18 (m, 2H), 6.41 (s, |
| | 1H), 4.56 (q, 2H), 3.40 (m, 4H), 1.96 (m, 1H) 1.34 (t, 3H), |
| | 1.24 (m, 6H) 0.94 (m, 4H). |
| 20 | δ 8.82 (dd, 1H), 8.00 (br s, 1H, NH), 7.18 (m, 2H), 6.41 (s, |
| | 1H), 6.24 (br s, 1H, NH) 4.56 (q, 2H), 3.42 (q, 2H), 1.96 |
| | (m, 1H) 1.34 (t, 3H), 1.26 (t, 3H), 0.98 (m, 2H), 0.80 (m, |
| | 2H). |
| 21 | δ 8.36 (br s, 1H, NH), 8.20 (dd, 1H), 7.12 (m, 2H), 6.42 (s, |
| | 1H), 5.80 (m, 1H), 5.24 (m, 2H), 4.56 (q, 2H), 4.16 (m, |
| | 2H), 3.10 (s, 3H), 1.98 (m, 1H) 1.42 (t, 3H), 1.26 (t, 3H) |
| | 0.94 (m, 2H, 0.80 (m, 2H). |
| 22 | δ 9.00 (br s, 1H, NH), 8.62 (dd, 1H), 7.18 (m, 2H), 6.56 (s, |
| | 1H), 4.18 (q, 2H), 3.10 (s, 3H), 3.06 (s, 3H), 2.42 (d, 2H), |
| | 1.98 (m, 1H) 1.46 (t, 3H), 0.98 (d, 6H). |
| 23 | δ 9.00 (br s, 1H, NH), 8.60 (dd, 1H), 7.18 (m, 2H), 6.52 (s, |
| | 1H), 4.14 (q, 2H), 3.40 (m, 4H), 2.44 (d, 2H), 1.98 (m, 1H) |
| | 1.46 (t, 3H, 1.20 (t, 6H), 0.98 (d, 6H). |
| 24 | δ 9.04 (br s, 1H, NH), 8.78 (dd, 1H), 7.62 (m, 1H), 7.20 (t, |
| | 1H), 6.60 (s, 1H), 6.42 (br s, 1H, NH), 4.18 (q, 2H), 3.62 |
| | (q, 2H), 2.46 (d, 2H), 1.98 (m, 1H) 1.48 (t, 3H), 1.22 (t, |
| | 3H), 1.02 (d, 6H). |
| 25 | δ 9.00 (br s, 1H, NH), 8.60 (dd, 1H), 7.62 (m, 1H), 7.20 |
| | (m, 2H), 6.60 (s, 1H), 5.82 (m, 1H), 5.28 (m, 2H), 4.12 (q, |
| | 2H), 3.00 (s, 3H), 2.52 (d, 2H), 1.98 (m, 1H) 1.48 (t, 3H), |
| | 1.00 (d, 6H). |
| 31 | δ 8.60 (1H, NH), 8.00 (d, 1H), 7.90 (dd, 1H), 7.40 (m, |
| | 2H), 6.71 (s, 1H), 6.20 (t, 1H, NH), 5.22 (m, 2H, 3.42 (m, |
| | 2H), 1.35 (s, 9H), 1.22 (t, 3H). |
| 32 | δ 9.23 (1H, NH), 7.64 (dd, 1H), 7.30 (m, 2H), 7.23 (dd, |
| | 1H), 6.91 (s, 1H), 5.33 (m, 2H), 3.62 (q, 2H), 3.20 (q, 2H), |
| | 1.35 (s, 9H), 1.22 (t, 3H), 1.10 (t, 3H). |
| 33 | δ 9.00 (1H, NH), 8.00 (m, 2H), 7.45 (m, 2H), 6.71 (s, 1H), |
| | 6.42 (t, 1H, NH), 5.82 (d, 1H), 3.46 (q, 2H), 1.36 (t, 3H), |
| | 1.28 (s, 9H). |
| 34 | δ 9.20 (1H, NH), 8.00 (m, 1H), 7.60 (dd, 1H), 7.20 (m, |
| | 2H), 6.91 (dd, 1H), 6.87 (s, 1H), 5.82 (d, 1H), 3.56 (q, |
| | 2H), 3.20 (q, 2H), 1.35 (s, 9H), 1.22 (t, 6H). |
| 35 | δ 8.80 (s, 1H, NH), 8.00 (d, 1H), 7.90 (d, 1H), 7.42 (m, |
| | 2H), 6.70 (s, 1H), 6.40 (br s, 1H, NH), 4.82 (m, 4H), 4.18 |
| | (m, 2H), 1.31 (s, 9H), 1, 18 (t, 3H). |
| 36 | δ 9.20 (s, 1H, NH), 7.70 (dd, 1H), 7.32 (m, 2H), 6.92 (dd, |
| | 1H), 6.80 (s, 1H), 4.82 (m, 4H), 3.48 (m, 4H), 1.31 (s, 9H), |
| | 1,25 (t, 6H). |
| 37 | δ 8.20 (br s, 1H, NH), 7.86 (m, 3H), 7.45 (dd, 1H, 7.40 (t, |
| | 1H), 6.45 (s, 1H), 5.82 (d, 1H), 5.00 (d, 1H), 4.06 (m, 2H), |
| | 1.28 (s, 9H). |
| 38 | δ 8.06 (m, 3H, 7.45 (m, 2H), 6.55 (s, 1H), 5.87 (d, 1H), |
| | 5.00 (d, 1H), 3.86 (m, 4H, 1.28 (s, 9H). |
| 39 | δ 8.26 (br s, 1H, NH), 7.86 (m, 3H), 7.45 (m, 2H), 6.65 (s, |
| | 1H), 6.40 (br s, 1H, NH), 5.82 (d, 1H), 5.00 (d, 1H), 2.80 |
| | (m, 1H), 1.28 (s, 9H), 0.90 (m, 2H), 0.60 (m, 2H). |
| 44 | δ 7.44 (m, 3H), 7.12 (dd, 1H), 6.76 (s, 1H), 4.60 (q, 2H), |
| | 2.86 (m, 1H), 1.42 (t, 3H), 1.38 (s, 9H), 0.86 (m, 2H), 0.68 |
| | (m, 2H). |
| 45 | δ 7.44 (m, 3H), 7.12 (dd, 1H), 6.70 (s, 1H), 4.60 (m, 4H), |
| | 3.82 (m, 2H), 1.42 (t, 3H), 1.38 (s, 9H). |
| 46 | δ 7.42 (m, 3H), 7.10 (dd, 1H), 6.80 (s, 1H), 6.52 (s, 1H, |
| | NH), 5.92 (m, 1H), 5.20 (m, 2H), 4.60 (m, 2H), 4.02 (m, |
| | 2H), 1.42 (t, 3H), 1.38 (s, 9H). |
| 47 | δ 7.42 (m, 3H), 7.10 (dd, 1H), 6.80 (s, 1H), 6.52 (s, 1H, |
| | NH), 4.60 (m, 2H), 4.20 (m, 2H), 2.20 (m, 1H), 1.42 (t, |
| | 3H), 1.38 (s, 9H). |
| 48 | (Acetone-d₆) δ 1.18 (t, 3H), 1.3 (s, 9H), 1.4 (t, 3H), 3.0 (s, |
| | 3H), 3.4 (m, 2H), 4.5 (q, 2H), 6.9 (s, 1H), 7.1 (t, 1H), 7.4 |
| | (d, 1H), 7.8 (d, 1H), 7.9 (s, 1H), 9.5 (br s, 1H). |
| 49 | (Acetone-d₆) δ 1.29 (s, 9H), 1.39 (t, 3H), 3.0 (m, 6H, 4.5 |
| | (q, 2H), 6.8 (s, 1H), 7.1 (d, 1H), 7.4 (t, 1H), 7.8 (d, 1H), |
| | 7.9 (s, 1H), 9.5 (br s, 1H). |
| 50 | (DMSO-d₆) δ 1.13 (t, 3H), 1.29 (s, 9H), 3.3 (q, 2H), 5.31 |
| | (d, 2H), 7.0 (s, 1H), 7.4 (t, 1H), 7.59 (d, 1H), 7.89 (d, 1H), |
| | 8.16 (s, 1H), 8.47 (t, 1H), 10.3 (br s, 1H). |
| 51 | δ 7.82 (s, 1H), 7.80 (dd, 1H), 7.62 (s, 1H, NH), 7.40 (m, |
| | 2H), 6.48 (s, 1H), 6.00 (s, 1H, NH), 4.58 (q, 2H), 4.24 (m, |
| | 1H), 1.42 (t, 3H), 1.38 (s, 9H), 1.26 (d, 3H), 1.20 (d, 3H). |
| 52 | δ 8.02 (d, 1H), 7.72 (d, 1H), 7.50 (s, 1H), 7.42 (t, 1H), |
| | 6.45 (s, 1H), 6.05 (s, 1H), 4.56 (q, 2H), 3.58 (m, 2H), 2.95 |
| | (t, 2H), 1.44 (t, 3H), 1.34 (s, 9H). |
| 53 | δ 8.02 (d, 1H), 7.68 (d, 1H), 7.65 (s, 1H), 7.40 (t, 1H), |
| | 6.54 (s, 1H), 6.28 (s, 1H), 4.15 (s, 3H), 3.56 (d of t, 1H), |
| | 2.94 (t, 2H), 1.34 (s, 9H). |
| 54 | δ 7.85 (d, 1H), 7.57 (t, 1H), 7.39 (d, 1H), 6.22 (s, 1H), |
| | 4.29 (s, 1H), 3.99 (s, 3H), 3.18 (s, 2H), 1.22 (s, 9H). |
| 56 | (Acetone-d₆) δ 1.2 (t, 3H), 1.30 (s, 9H), 3.4 (q, 2H), |
| | 5.0-5.1 (m, 1H). 5.17 (dd, 2H), 6.0-6.1 (m, 1H), 6.96 (s, |
| | 1H), 7.4 (t, 1H), 7.6 (d, 1H), 7.7 (br s, 1H), 7.9 (d, 1H), 8.2 |
| | (s, 1H), 9.5 (br s, 1H). |
| 58 | δ 8.20 (m, 1H), 7.96 (m, 1H), 7.80 (br s, 1H, NH), 7.20 |
| | (m, 1H), 6.49 (s, 1H), 4.62 (q, 2H), 3.50 (q, 2H), 1.42 (t, |
| | 3H), 1.32 (s, 9H), 1.26 (t, 3H). |
| 59 | δ 9.32 (br s, 1H, NH), 7.66 (m, 1H), 7.18 (dd, 1H), 7.00 (t, |
| | 1H), 6.69 (s, 1H), 4.62 (q, 2H), 3.60 (q, 2H), 3.34 (q, 2H), |
| | 1.42 (t, 6H), 1.32 (s, 9H), 1.26 (t, 3H). |
| 60 | δ 8.30 (br s, 1H, NH), 8.16 (m, 1H), 7.90 (m, 1H), 7.20 (t, |
| | 1H), 6.59 (s, 1H), 4.62 (m, 4H), 3.80 (m, 2H), 1.42 (t, 3H), |
| | 1.32 (s, 9H). |
| 61 | δ 8.32 (s, 1H, NH), 7.86 (m, 2H), 7.58 (m, 2H), 6.58 (s, |
| | 1H), 6.30 (s, 1H, NH), 4.58 (q, 2H), 3.24 (t, 2H), 1.92 (m, |
| | 1H), 1.42 (t, 3H), 1.31 (s, 9H), 0.96 (d, 6H). |
| 62 | δ 8.38 (s, 1H, NH), 7.86 (m, 2H), 7.58 (m, 2H), 6.58 (s, |
| | 1H), 6.30 (s, 1H, NH), 4.58 (q, 2H), 3.28 (m, 2H), 1.92 (m, |
| | 1H), 1.42 (t, 3H), 1.31 (s, 9H), 1.00 (m, 1H), 0.66 (m, 2H), |
| | 0.32 (m, 2H). |
| 63 | δ 7.78 (s, 1H), 7.73 (d, 1H), 7.58 (d, 1H), 7.58 (t, 1H), |
| | 6.53 (s, 1H), 4.57 (q, 2H), 4.43 (s, 2H), 3.67 (d, 2H), 1.45 |
| | (t, 3H), 1.34 (s, 9H) 1.27 (t, 3H). |
| 64 | δ 7.72 (d, 1H), 7.69 (s, 1H), 7.57 (d, 1H), 7.58 (t, 1H), |
| | 6.53 (s, 1H), 4.57 (q, 2H), 4.37 (s, 2H), 2.94 (m, 1H), 1.45 |
| | (t, 3H), 1.34 (s, 9H) 0.97 (m, 2H). |
| 65 | δ 7.80 (s, 1H), 7.73 (d, 1H), 7.71 (d, 1H), 7.47 (t, 1H), |
| | 6.54 (s, 1H), 4.59 (q, 2H), 4.42 (s, 2H), 3.58 (t, 2H), 1.66 |
| | (m, 2H), 1.45 (t, 3H), 1.35 (s, 9H), 0.95 (t, 3H). |
| 66 | δ 7.75 (d, 1H), 7.66 (s, 1H), 7.55 (d, 1H), 7.48 (t, 1H), |
| | 6.52 (s, 1H), 4.76 (m, 1H), 4.57 (q, 2H), 4.39 (s, 2H), 2.00 |
| | (m, 2H), 2.70 (m, 6H), 1.45 (t, 3H), 1.35 (s, 9H). |
| 67 | δ 9.58 (s, 1H), 7.87 (s, 1H), 7.86 (d, 1H), 7.15 (t, 1H), 6.71 |
| | (s, 1H, 4.34 (q, 2H), 3.62 (s, 3H), 1.12 (t, 3H), 1.11 (s, |
| | 9H). |
| 68 | δ 9.24 (s, 1H), 7.57 (s, 1H), 7.55 (d, 1H, 7.04 (d, 1H, 6.70 |
| | (s, 1H), 6.70 (br s, 1H), 4.45 (q, 2H), 3.33 (q, 2H), 2.28 (s, |
| | 3H, 1.30 (t, 3H), 1.22 (t, 3H), 1.12 (s, 9H). |
| 69 | δ 8.23 (s, 1H), 7.58 (d, 1H), 7.22 (s, 1H), 7.17 (d, 1H, 6.62 |
| | (s, 1H), 4.56 (q, 2H), 3.8-3.6 (br m, 2H), 3.16 (br q, 2H), |
| | 2.20 (s, 3H), 1.43 (t, 3H), 1.33 (s, 9H), 1.25 (t, 3H), 1.06 |
| | (t, 3H). |
| 71 | δ 8.86 (dd, 1H), 7.80 (m, 1H), 7.16 (m, 1H), 6.42 (s, 1H, |
| | 4.58 (q, 2H, 1.51 (s, 9H), 1.42 (t, 3H), 1.32 (s, 9H). |
| 72 | δ 9.04 (br s, 1H, NH), 8.78 (dd, 1H), 7.62 (m, 1H), 7.20 (t, |
| | 1H), 6.60 (s, 1H), 4.18 (q, 2H, 2.46 (d, 2H), 1.98 (m, 1H) |
| | 1.48 (t, 3H), 1.02 (d, 6H). |
| 73 | δ 8.62 (dd, 1H), 7.92 (br s, 1H, NH), 7.66 (m, 1H), 7.24 |
| | (m, 1H), 6.48 (s, 1H, 4.56 (q, 4H), 4.18 (q, 2H), 3.00 (m, |
| | 1H), 1.31 (t, 3H), 1.28 (t, 3H), 1.24 (d, 6H). |
| 74 | δ 8.52 (dd, 1H), 7.80 (br s, 1H, NH), 7.62 (m, 1H), 7.28 |
| | (m, 1H), 6.48 (s, 1H,), 4.56 (q, 4H), 4.20 (q, 2H, 3.00 (m, |
| | 1H), 1.31 (t, 3H, 1.28 (t, 3H), 1.24 (d, 6H). |
| 75 | δ 8.66 (d, 1H), 7.26 (s, 1H), 6.78 (d, 1H), 6.47 (s, 1H), |
| | 4.57 (q, 2H), 3.90 (s, 3H, 3.89 (s, 3H), 1.44 (t, 3H), 1.33 |
| | (s, 9H). |
| 76 | δ 8.57 (d, 1H), 7.60 (s, 1H), 6.78 (d, 1H), 6.47 (s, 1H), |
| | 4.56 (q, 2H), 4.36 (q, 2H), 3.89 (s, 3H, 1.44 (t, 3H), 1.38 |
| | (t, 3H), 1.33 (s, 9H. |
| 77 | δ 8.55 (d, 1H), 7.57 (s, 1H), 6.78 (d, 1H), 6.47 (s, 1H), |
| | 5.22 (septet, 1H), 4.56 (q, 2H), 4.36 (q, 2H), 3.89 (s, 3H), |
| | 1.47 (t, 3H), 1.34 (d, 6H, 1.33 (s, 9H). |
| 78 | δ 8.00 (m, 4H), 7.45 (t, 1H), 6.61 (s, 1H), 5.86 (d, 1H, |
| | 4.92 (d, 1H), 4.40 (q, 2H), 1.36 (t, 3H), 1.28 (s, 9H). |
| 79 | δ 8.00 (s, 1H), 8.00 (dd, 1H), 7.90 (d, 1H), 7.42 (t, 1H, |
| | 6.54 (s, 1H), 6.40 (br s, 4.92 (m, 4H), 4.40 (q, 2H), 1.36 |
| | (t, 3H), 1.28 (s, 9H). |
| 81 | (DMSO-d₆) δ 1.29 (s, 9H), 1.3 (t, 3H), 3.88 (s, 3H), 4.4 (q, |
| | 2H), 7.0 (s, 1H), 7.5 (t, 1H), 7.7 (d, 1H), 8.0 (d, 1H), 8.4 |
| | (s, 1H), 10.3 (br s, 1H). |
| 83 | δ 8.04 (d, 1H), 7.80 (d, 1H), 7.42 (t, 1H), 6.52 (s, 1H), |
| | 4.73 (q, 2H), 4.58 (m, 2H), 1.42 (t, 3H), 1.38 (s, 9H). |
| 84 | δ 8.34 (s, 1H), 8.04 (d, 1H), 7.82 (d, 1H), 7.42 (t, 1H, 6.62 |
| | (s, 1H), 4.90 (d, 2H), 4.62 (q, 2H), 2.52 (t, 1H), 1.42 (t, |
| | 3H), 1.38 (s, 9H). |
| 85 | δ 8.24 (s, 1H), 8.06 (d, 1H), 7.82 (dd, 1H), 7.42 (t, 1H), |
| | 6.58 (s, 1H), 6.00 (m, 1H), 5.32 (m, 2H), 4.80 (d, 2H), |
| | 4.60 (q, 2H), 2.52 (t, 1H), 1.42 (t, 3H), 1.38 (s, 9H). |
| 86 | δ 8.24 (s, 1H), 8.06 (d, 1H), 7.82 (dd, 1H), 7.42 (t, 1H), |
| | 6.58 (s, 1H), 4.60 (d, 2H), 4.32 (t, 2H), 1.76 (m, 2H), 1.42 |
| | (t, 3H), 1.38 (s, 9H). |
| 87 | δ 7.90 (d, 1H), 7.72 (d, 2H), 7.55 (s, 1H), 7.30 (t, 1H), |
| | 6.50 (s, 1H), 4.56 (q, 2H), 3.91 (s, 3H), 2.52 (s, 3H, 1.44 |
| | (t, 3H), 1.34 (s, 9H. |
| 88 | δ 7.92 (d, 1H), 7.71 (d, 2H), 7.52 (s, 1H), 7.30 (t, 1H), |
| | 6.47 (s, 1H), 4.57 (q, 2H), 4.37 (q, 2H), 2.52 (s, 3H), 1.42 |
| | (t, 3H), 1.40 (t, 3H), 1.34 (s, 9H). |
| 89 | δ 8.00 (m, 2H), 7.70 (br s, 1H, NH), 7.18 (t, 1H), 6.48 (s, |
| | 1H,), 4.56 (q, 4H), 4.42 (q, 2H), 1.31 (m, 6H), 1.24 (s, |
| | 9H). |
| 90 | δ 7.93 (d, 1H), 7.73 (d, 1H), 7.53 (s, 1H), 7.30 (t, 1H), |
| | 6.47 (s, 1H), 4.57 (q, 2H), 4.27 (t, 2H), 2.53 (s, 3H), 1.80 |
| | (m, 2H), 1.44 (t, 2H), 1.35 (s, 9H), 1.04 (t, 3H). |
| 91 | δ 7.95 (d, 1H), 7.75 (d, 1H), 7.75 (s, 1H), 7.32 (t, 1H), |
| | 6.48 (s, 1H), 4.80 (m, 1H), 4.65-4.50 (m, 5H), 4.59 (q, |
| | 2H), 2.53 (s, 3H), 1.45 (t, 3H), 1.35 (s, 9H). |
| 92 | δ 7.90 (d, 1H), 7.68 (d, 1H), 7.52 (s, 1H), 7.30 (t, 1H), |
| | 6.47 (s, 1H), 5.23 (septet, 1H), 4.57 (q, 2H), 2.52 (s, 3H), |
| | 1.42 (t, 2H, 1.37 (d, 6H), 1.35 (s, 9H). |
| 93 | δ 7.92 (d, 1H), 7.73 (d, 1H), 7.54 (s, 1H), 7.31 (t, 1H), |
| | 4.57 (q, 2H), 4.15 (d, 2H), 2.53 (s, 3H), 1.44 (t, 2H), 1.35 |
| | (s, 9H), 1.29 (m, 1H), 0.64 (m, 2H), 0.36 (m, 2H). |
| 94 | δ 7.93 (d, 1H), 7.75 (d, 1H), 7.53 (s, 1H), 7.31 (t, 1H), |
| | 6.47 (s, 1H), 6.05 (dd of t, 1H), 5.42 (d, 1H), 5.25 (d, 1H), |
| | 4.82 (m, 2H), 4.57 (q, 2H), 2.53 (s, 3H), 1.44 (t, 2H), 1.35 |
| | (s, 9H). |
| 95 | δ 7.86 (d, 1H), 7.78 (d, 1H), 7.63 (s, 1H), 6.97 (d, 1H), |
| | 6.45 (s, 1H), 4.56 (q, 2H), 4.36 (q, 2H), 4.11 (q, 2H), 1.40 |
| | (m, 9H), 1.32 (s, 9H). |
| 96 | δ 7.86 (dd, 1H), 7.59 (s, 1H), 6.97 (d, 1H), 6.45 (s, 1H), |
| | 4.56 (q, 2H), 4.36 (q, 2H), 3.91 (s, 3H), 1.44 (t, 3H), 1.39 |
| | (t, 3H), 1.33 (s, 9H). |
| 97 | δ 8.60 (d, 1H), 8.43 (s, 1H), 7.62 (d, 1H), 7.20 (t, 1H), |
| | 6.44 (s, 1H), 4.60 (q, 2H), 4.42 (q, 2H), 3.94 (s, 3H), 1.46 |
| | (t, 3H), 1.42 (t, 3H), 1.34 (s, 9H). |
| 98 | δ 8.04 (d, 1H), 7.94 (br s, 1H, NH), 7.80 (m, 2H), 7.40 (t, |
| | 1H), 6.48 (s, 1H), 4.58 (q, 4H), 1.61 (s, 9H), 1.44 (t, 3H), |
| | 1.33 (s, 9H). |
| 99 | δ 7.92 (s, 1H), 7.80 (d, 1H), 7.65 (s, 1H), 7.24 (d, 1H), |
| | 6.47 (s, 1H), 4.56 (q, 2H), 4.37 (q, 2H), 2.57 (s, 3H), 1.44 |
| | (t, 3H), 1.40 (t, 3H), 1.33 (s, 9H). |
| 100 | δ 7.85 (d, 1H), 7.83 (s, 1H), 7.64 (s, 1H), 7.24 (d, 1H), |
| | 6.47 (s, 1H), 5.25 (septet, 1H), 4.56 (q, 2H), 2.57 (s, 3H), |
| | 1.44 (t, 3H), 1.38 (d, 6H), 1.33 (s, 9H). |
| 101 | δ 8.30 (s, 1H), 7.87 (d, 1H), 7.77 (s, 1H), 6.92 (d, 1H), |
| | 6.58 (s, 1H), 5.21 (septet, 1H), 4.56 (q, 2H), 3.84 (s, 3H), |
| | 1.42 (t, 3H), 1.32 (d, 6H), 1.28 (s, 9H). |
| 107 | (DMSO-d₆) δ 1.0 (s, 9H), 2.0 (s, 3H), 3.38 (s, 3H), 3.8 |
| | (s, 3H), 5.4 (s, 1H), 7.2 (d, 1H), 7.3 (t, 1H), 7.4 (s, 1H), |
| | 7.5 (d, 1H), 11.3 (s, 1H). |
| 110 | δ 8.7 (br s, 1H), 7.83-7.88 (m, 2H), 7.36-7.38 (m, 2H), |
| | 6.6 (s, 1H), 4.7 (m, 1H), 2.3 (s, 1H), 1.55 (s, 3H), 1.53 (s, |
| | 3H), 1.4 (s, 9H). |
| 111 | δ 7.96 (d, 1H), 7.55 (d, 1H), 7.47 (s, 1H), 7.34 (t, 1H), |
| | 6.45 (s, 1H), 4.16 (s, 1H), 3.04 (m, 4H), 3.18 (s, 2H), 1.34 |
| | (s, 9H). |
| 114 | δ 11.20 (s, 1H), 10.30 (s, 1H), 8.14 (s, 1H), 7.94 (m, 1H), |
| | 7.74 (m, 1H), 7.40 (m, 2H), 7.11 (s, 1H), 5.68 (d, 1H), |
| | 4.90 (d, 1H), 2.15 (s, 3H), 1.32 (s, 9H). |
| 115 | δ 8.00 (m, 2H), 7.64 (s, 1H), 7.40 (m, 2H), 6.54 (s, 1H), |
| | 4.91 (m, 4H), 2.28 (s, 3H), 1.32 (s, 9H) |
| 116 | δ 7.88 (d, 1H), 7.65 (d, 1H), 7.47 (s, 1H), 7.27 (t, 1H), |
| | 6.48 (s, 1H), 4.16 (s, 1H), 2.81 (t, 2H), 2.71 (t, 2H), 1.90 |
| | (m, 2H), 3.18 (s, 2H), 1.34 (s, 9H). |
| 117 | (DMSO-d₆) δ 11.2 (s, 1H), 10.2 (br s, 1H), 8.0 (s, 1H), |
| | 7.7 (d, 1H), 7.35-7.38 (m, 3H), 7.0 (s, 1H), 6.0 (m, 1H), |
| | 5.1 (d, 2H), 4.9 (d, 1H), 2.51 (s, 3H), 1.29 (s, 9H). |
| 124 | δ 8.04 (m, 2H), 7.60 (dd, 1H), 7.46 (t, 2H), 6.64 (s, 1H), |
| | 4.16 (s, 3H), 3.40 (m, 1H), 1.32 (s, 9H), 1.13 (d, 6H). |
| 125 | δ 8.36 (s, 1H), 8.16 (dd, 1H), 7.94 (s, 1H), 7.60 (m, 1H), |
| | 7.52 (t, 1H), 6.54 (s, 1H) 4.15 (s, 3H), 3.00 (m, 2H), 1.35 |
| | (m, 4H), 1.28 (s, 9H), 0.90 (t, 3H). |
| 127 | δ 8.14 (s, 1H), 7.94 (m, 3H), 7.64 (m, 1H), 7.44 (t, 1H), |
| | 6.61 (s, 1H), 5.88 (d, 1H), 4.93 (d, 1H), 2.65 (s, 3H), 1.32 |
| | (s, 9H). |
| 128 | δ 8.10 (m, 1H), 8.00 (m, 1H), 7.90 (br s, 1H), 7.60 (dd, |
| | 1H), 7.42 (t, 1H), 6.56 (s, 1H), 4.71 (m, 4H), 2.62 (s, 3H), |
| | 1.32 (s, 9H). |
| 129 | (DMSO-d₆) δ 10.3 (br s, 1H), 8.0 (d, 1H), 7.7 (d, 1H), 7.5 |
| | (t, 1H), 7.0 (s, 1H), 6.0 (m, 1H), 5.1 (d, 2H), 4.9 (d, 1H), |
| | 2.58 (s, 3H), 1.29 (s, 9H). |
| 130 | δ 7.82 (s, 1H), 7.80 (dd, 1H), 7.62 (s, 1H, NH), 7.40 (d, |
| | 2H), 6.48 (s, 1H, 4.58 (q, 2H, 1.42 (t, 3H), 1.38 (s, 9H). |
| 131 | δ 8.42 (s, 1H, NH), 8.20 (dd, 1H), 8.00 (s, 1H, NH), 7.74 |
| | (d, 1H), 7.40 (t, 1H), 6.68 (s, 1H), 4.58 (q, 2H), 2.92 (s, |
| | 3H), 1.42 (t, 3H), 1.38 (s, 9H). |
| 132 | δ 8.40 (s, 1H, NH), 8.20 (dd, 1H), 7.80 (s, 1H, NH), 7.74 |
| | (d, 1H), 7.40 (t, 1H), 6.68 (s, 1H), 4.40 (m, 4H), 1.38 (s, |
| | 9H). |
| 133 | δ 8.42 (s, 1H, NH), 8.20 (dd, 1H), 8.00 (s, 1H, NH), 7.74 |
| | (d, 1H), 7.40 (t, 1H), 6.68 (s, 1H), 4.58 (q, 2H), 3.22 (s, |
| | 3H), 1.42 (t, 3H), 1.38 (s, 9H). |
| 134 | δ 8.22 (s, 1H, NH), 8.20 (dd, 1H), 8.00 (s, 1H, NH), 7.74 |
| | (d, 1H), 7.40 (t, 1H), 6.68 (s, 1H), 4.58 (q, 2H), 1.42 (t, |
| | 3H), 1.38 (s, 9H). |
| 135 | δ 7.86 (s, 1H), 7.80 (dd, 1H), 7.60 (s, 1H, NH), 7.40 (d, |
| | 2H), 6.48 (s, 1H), 4.58 (q, 2H), 1.42 (t, 3H), 1.38 (s, 9H). |
| 136 | δ 7.62 (s, 1H, NH), 7.36 (m, 2H), 7.08 (dd, 1H,), 6.86 (dd, |
| | 1H), 6.60 (dd, 1H), 6.42 (s, 1H), 4.58 (q, 2H), 4.00 (q, |
| | 2H), 1.42 (m, 6H), 1.31 (s, 9H). |
| 137 | δ 7.60 (s, 1H, NH), 7.364 (m, 2H), 7.08 (dd, 1H,), 6.86 |
| | (dd, 1H), 6.60 (dd, 1H), 6.40 (s, 1H), 4.58 (q, 2H), 4.00 (t, |
| | 2H), 1.90 (m, 2H), 1.52 (m, 2H), 1.42 (t, 3H), 1.31 (s, 9H), |
| | 0.97 (s, 3H). |
| 138 | δ 7.62 (s, 1H, NH), 7.36 (m, 1H), 7.28 (m, 1H,), 7.00 (m, |
| | 1H), 6.60 (dd, 1H), 6.40 (s, 1H), 4.58 (q, 2H), 4.24 (t, 2H), |
| | 2.20 (m, 2H), 1.52 (m, 2H), 1.42 (t, 3H), 1.31 (s, 9H). |
| 139 | δ 8.01 (s, 1H), 7.80 (m, 2H), 7.22 (m, 2H), 7.20 (t, 1H), |
| | 6.52 (s, 1H), 4.56 (q, 2H), 1.43 (t, 3H), 1.33 (s, 9H). |
| 141 | δ 7.92 (s, 1H), 7.80 (d, 1H), 7.65 (s, 1H), 7.24 (d, 1H), |
| | 6.47 (s, 1H), 4.56 (q, 2H), 4.37 (q, 2H), 2.57 (s, 3H), 1.44 |
| | (t, 3H), 1.40 (t, 3H), 1.33 (s, 9H). |
| 142 | δ 8.60 (s, 1H, NH), 8.46 (s, 1H), 8.40 (d, 1H), 7.20 (d, |
| | 1H), 6.60 (s 1H), 5.90 (m, 1H), 5.80 (m, 1H), 4.54 (q, 2H), |
| | 4.40 (m, 2H), 4.20 (m, 2H), 1.42 (t, 3H), 1.31 (s, 9H). |
| 144 | δ 8.42 (d, 1H), 7.92 (m, 2H), 7.80 (s, 1H, NH), 6.60 (s, |
| | 1H), 4.60 (q, 2H), 3.40 (q, 2H), 1.40 (t, 3H), 1.31 (s, 9H). |
| 145 | δ 8.50 (s, 1H, NH), 8.22 (d, 1H), 7.80 (t, 1H), 7.30 (d, |
| | 1H), 6.60 (s, 1H), 4.60 (q, 2H), 3.56 (q, 2H), 3.32 (q, 2H), |
| | 1.43 (s, 9H), 1.30 (t, 3H), 1.15 (t, 3H). |
| 146 | δ 8.39 (d, 1H), 8.20 (s, 1H, NH), 7.92 (d, 1H), 7.84 (t, |
| | 1H), 7.80 (s, 1H), 6.60 (s, 1H), 4.60 (q, 2H), 2.96 (m, 1H), |
| | 1.40 (t, 3H), 1.31 (s, 9H), 0.88 (m, 2H), 0.68 (m, 2H). |
| 147 | δ 8.42 (m, 2H), 7.92 (m, 3H, NH), 6.60 (s, 1H), 4.60 (q, |
| | 2H), 4.20 (m, 2H), 2.22 (m, 1H), 1.46 (t, 3H), 1.31 (s, 9H). |
| 148 | δ 8.62 (s, 1H), 8.32 (s, 1H), 7.42 (s, 1H), 6.60 (s, 1H), 4.60 |
| | (q, 2H), 3.96 (s, 3H), 2.52 (s, 3H), 1.46 (t, 3H), 1.31 (s, |
| | 9H). |
| 149 | δ 8.52 (s, 1H), 8.32 (s, 1H), 7.42 (s, 1H), 6.60 (s, 1H), 6.42 |
| | (t, 1H, NH), 4.60 (q, 2H), 3.40 (q, 2H), 2.52 (s, 3H), 1.46 |
| | (t, 3H), 1.31 (s, 9H). |
| 150 | δ 8.22 (s, 1H), 8.18 (s, 1H), 7.82 (s, 1H), 7.64 (t, 1H), 6.57 |
| | (s, 1H), 4.60 (q, 2H), 3.40 (q, 2H), 2.52 (s, 3H), 1.46 (t, |
| | 3H), 1.31 (s, 9H). |
| 152 | δ 8.60 (s, 1H, NH), 8.38 (d, 2H), 7.60 (d, 1H), 6.58 (s, |
| | 1H), 6.40 (t, 1H, NH), 4.58 (q, 2H), 3.60 (q, 2H), 1.45 (t, |
| | 3H), 1.31 (s, 9H), 1.28 (t, 3H). |
| 153 | δ 8.60 (s, 1H, NH), 8.38 (d, 2H), 7.04 (d, 1H), 6.58 (s, |
| | 1H), 4.58 (q,. 2H), 3.60 (q, 2H), 3.28 (q, 2H), 1.40 (t, 3H), |
| | 1.31 (s, 9H), 1.28 (t, 3H). |
| 154 | δ 8.60 (s, 1H, NH), 8.40 (d, 2H), 7.60 (d, 1H), 6.58 (s, |
| | 1H), 6.40 (t, 1H, NH), 4.58 (q, 2H), 3.40 (t, 2H), 1.96 (m, |
| | 1H), 1.31 (s, 9H), 1.28 (t, 3H), 1.4 (d, 6H). |
| 155 | δ 8.60 (s, 1H, NH), 8.40 (d, 2H), 7.60 (d, 1H), 6.58 (s, |
| | 1H), 6.50 (t, 1H, NH), 4.58 (q, 2H), 3.36 (t, 2H), 1.90 (m, |
| | 1H), 1.31 (s, 9H), 1.30 (t, 3H), 1.28 (t, 3H), 0.62 (m, 2H), |
| | 0.36 (m, 2H). |
| 157 | δ 8.80 (s, 1H), 8.42 (d, 1H), 7.64 (d, 1H), 6.58 (s, 1H), |
| | 4.58 (q, 2H), 4.26 (q, 2H), 1.45 (m, 6H), 1.21 (s, 9H). |
| 158 | δ 8.42 (m, 2H), 7.92 (m, 3H, NH & aromatic), 6.60 (s, |
| | 1H), 4.60 (q, 2H), 4.20 (m, 2H), 3.12 (s, 3H), 2.22 (m, |
| | 1H), 1.46 (t, 3H), 1.31 (s, 9H). |
| 159 | δ 8.68 (s, 1H, NH), 8.40 (d, 2H), 7.04 (d, 1H), 6.61 (s, |
| | 1H), 5.86 (m, 1H), 5.22 (m, 2H), 4.58 (q, 2H), 4.20 (d, |
| | 1H), 3.80 (d, 1H), 3.08 (s, 3H), 1.42 (t, 3H), 1.31 (s, 9H). |
| 160 | δ 8.80 (s, 1H, NH), 8.40 (d, 1H), 7.14 (dd, 1H), 6.61 (s, |
| | 1H), 4.58 (q, 2H), 4.40 (d, 1H), 4.00 (d, 1H), 3.17 & 3.06 |
| | (2 × s, 3H), 2.20 (m, 1H), 1.42 (t, 3H), 1.31 (s, 9H). |
| 161 | δ 8.68 (s, 1H, NH), 8.52 (s, 1H), 8.40 (d, 1H), 7.46 (d, |
| | 1H), 6.80 (t, 1H, NH), 6.61 (s, 1H), 4.58 (q, 2H), 3.60 (m, |
| | 4H), 3.40 (s, 1H), 1.48 (t, 3H), 1.31 (s, 9H). |
| 163 | δ 8.46 (m, 2H), NH and 1 aromatic), 7.80 (m, 1H), 7.32 |
| | (dd, 1H), 6.55 (s, 1H), 5.82 (m, 1H), 5.34 (m, 2H), 4.60 (q, |
| | 2H), 4.20 (d, 1H), 3.92 (d, 1H), 3.09 (2 s, 3H), 1.43 (t, |
| | 3H), 1.30 (s, 9H). |
| 165 | δ 8.40 (d, 1H), 8.36 (br s, 1H), 6.58 (s, 1H), 6.48 (d, |
| | 1H), 4.60 (q, 2H), 3.99 (s, 3H), 1.45 (q, 3H), 1.30 (s, 9H). |
| 166 | δ 8.20 (br s, 1H), 6.51 (s, 1H), 5.80 (s, 1H), 4.60 (q, |
| | 2H), 3.96 (s, 6H), 1.45 (q, 3H), 1.30 (s, 9H). |
| 167 | δ 8.16 (br s, 1H), 6.56 (s, 1H), 6.02 (s, 1H), 5.00 (br s, |
| | 1H), 4.54 (q, 2H), 3.90 (s, 3H), 1.30 (s, 9H), 1.26 (t, 3H). |
| 169 | δ 8.74 (br s, 1H), 7.87 (s, 1H), 7.82 (d, 1H), 7.82 (d, 1H), |
| | 7.6 (m, 1H), 7.3 (t, 1H), 7.1 (d, 1H) 3.5 (m, 2H), 3.3 (m, |
| | 2H), 1.66 (s, 9H), 1.2 (m, 3H), 1.1 (m, 3H). |
| 170 | δ 8.83 (br s, 1H), 8.14 (s, 1H), 7.84-7.88 (m, 2H), 7.56 (d, |
| | 1H), 7.42 (t, 1H), 6.28 (br s, 1H), 3.4 (q, 2H), 1.6 (s, 9H), |
| | 1.27 (t, 3H). |
| 171 | δ 8.75 (br s, 1H), 7.86-7.89 (m, 3H), 7.36-7.39 (m, 2H), |
| | 2.3 (s, 3H), 1.66 (s, 9H). |
| 172 | δ 8.0 (s, 1H), 7.99 (s, 1H), 7.92 (br s, 1H), 7.7 (d, 1H), 7.5 |
| | (d, 1H), 7.4 (t, 1H), 6.2 (m, 1H), 4.2 (q, 2H), 3.5 (m, 2H), |
| | 1.5 (t, 3H), 1.2 (t, 3H). |
| 183 | δ 7.99 (s, 1H), 7.96 (s, 1H), 7.78 (d, 1H), 7.7 (br s, 1H), |
| | 7.47 (d, 1H) 7.4 (t, 1H), 6.2 (br s, 1H), 3.4 (q, 2H), 2.56 (s, |
| | 3H), 1.59 (s, 9H), 1.2 (t, 3H). |
| 184 | δ 8.0 (s, 1H), 7.88 (br s, 1H), 7.6 (d, 1H), 7.4 (s, 1H), 7.3 |
| | (t, 1H), 7.0 (d, 1H), 3.5 (m, 2H), 3.3 (m, 2H), 2.55 (s, 3H), |
| | 1.6 (s, 9H), 1.3 (m, 3H), 1.1 (m, 3H). |
| 185 | δ 8.0 (s, 1H), 7.9 (br s, 1H), 7.6 (d, 1H), 7.4 (s, 1H), 7.3 (t, |
| | 1H), 7.0 (d, 1H), 3.5 (m, 2H), 3.3 (m, 2H), 2.89 (t, 2H), |
| | 1.7 (q, 2H), 1.6 (s, 9H), 1.3 (m, 3H), 1.1 (m, 3H), 0.99 (t, |
| | 3H). |
| 186 | δ 8.0 (dd, 1H), 7.98 (s, 1H), 7.94 (s, 1H), 7.79 (dd, 1H), |
| | 7.55 (br s, 1H), 7.4 (t, 1H), 4.38 (q, 2H), 2.96 (q, 2H), 1.59 |
| | (s, 9H), 1.4 (t, 3H), 1.33 (t, 3H). |
| 187 | δ 8.02 (d, 1H), 8.0 (s, 1H), 7.96 (s, 1H), 7.7 (d, 1H), 7.6 |
| | (br s, 1H), 7.4 (t, 1H), 2.9 (q, 2H), 2.62 (s, 3H), 1.6 (s, |
| | 9H), 1.34 (t, 3H). |
| 188 | (DMSO-d₆) δ 11.2 (s, 1H), 9.6 (br s, 1H), 8.5 (s, 1H), 8.0 |
| | (d, 1H), 7.0-7.7 (m, 1H), 7.29-7.36 (m, 2H), 2.8 (q, 2H), |
| | 2.15 (s, 3H), 1.54 (s, 9H), 1.16 (t, 3H). |
| 189 | δ 7.9 (s, 1H), 7.34-7.36 (m, 2H), 7.2 (t, 1H), 7.0 (dd, 1H), |
| | 6.6 (dd, 1H), 4.5 (m, 1H), 2.55 (s, 3H), 1.59 (s, 9H), 1.35 |
| | (s, 3H), 1.33 (s, 3H). |
| 190 | δ 7.93 (s, 1H), 7.36-7.37 (m, 2H), 7.22 (t, 1H), 7.0 (dd, |
| | 1H), 6.6 (dd, 1H), 4.0 (q, 2H), 2.56 (s, 3H), 1.59 (s, 9H), |
| | 1.41 (t, 3H). |
| 191 | δ 8.2 (s, 1H), 7.8 (br s, 1H), 7.59-7.62 (m, 2H), 7.39 (t, |
| | 1H), 7.15 (d, 1H), 3.5 (m, 2H), 3.3 (m, 2H), 1.6 (s, 9H), |
| | 1.2 (m, 3H), 1.1 (m, 3H). |
| 192 | δ 8.18 (s, 1H), 8.0 (s, 1H), 7.8 (br s, 1H), 7.7 (d, 1H), 7.57 |
| | (d, 1H), 7.4 (t, 1H), 6.2 (br s, 1H), 3.5 (m, 1H), 1.6 (s, 9H), |
| | 1.26 (t, 3H). |
| 193 | δ 8.7 (dd, 1H), 7.94 (s, 1H), 7.65-7.7 (m, 2H), 7.15-7.22 |
| | (m, 1H), 6.2 (br s, 1H), 3.4 (m, 2H), 2.9 (q, 2H), 1.6 (s, |
| | 9H), 1.35 (t, 3H), 1.25 (t, 3H). |
| 194 | δ 8.79 (dd, 1H), 7.93 (s, 1H), 7.66-7.7 (m, 1H), 7.16-7.23 |
| | (m, 1H), 6.3 (br s, 1H), 3.0 (d, 3H), 1.6 (s, 9H), 1.35 (t, |
| | 3H). |
| 195 | δ 11.20 (s, 1H), 8.00 (s, 1H), 7.84 (d, 1H), 7.60 (s, 1H), |
| | NH), 7.46 (d, 1H), 7.40 (t, 1H), 2.73 (s, 3H), 2.16 (s, 3H), |
| | 1.45 (s, 9H). |
| 196 | δ 8.00 (s, 1H), 7.84 (d, 1H), 7.60 (s, 1H, NH), 7.46 (d, |
| | 1H), 7.40 (t, 1H), 2.73 (s, 3H), 2.24 (s, 3H), 1.45 (s, 9H). |
| 197 | δ 7.97 (s, 1H), 7.74 (d, 1H), 7.62 (s, 1H, NH), 7.46 (d, |
| | 1H), 7.40 (t, 1H), 3.40 (s, 3H), 2.73 (s, 3H), 1.45 (s, 9H). |
| 198 | δ 8.00 (s, 1H), 7.84 (d, 1H), 7.60 (s, 1H, NH), 7.46 (d, |
| | 1H), 7.40 (t, 1H), 6.26 (br s, 1H, NH), 3.50 (q, 2H), 2.73 |
| | (s, 3H), 1.45 (s, 9H), 1.20 (t, 3H). |
| 199 | δ 8.00 (s, 1H, NH), 7.64 (dd, 1H), 7.52 (d, 1H), 7.32 (t, |
| | 1H), 7.02 (dd, 1H), 3.50 (m, 2H), 3.28 (m, 2H), 2.71 (s, |
| | 3H), 1.45 (s, 9H), 1.22 (m, 6H). |
| 200 | δ 8.00 (s, 1H), 7.84 (d, 1H), 7.60 (m, 2H), NH & |
| | aromatic), 7.40 (t, 1H), 6.56 (br s, 1H, NH), 4.69 (t, 1H), |
| | 4.53 (t, 1H), 3.80 (m, 2H), 2.73 (s, 3H), 1.45 (s, 9H). |
| 201 | δ 8.00 (s, 1H), 7.84 (d, 1H), 7.60 (d, 1H, aromatic), 7.46 |
| | (s, 1H, NH), 7.36 (t, 1H), 6.46 (br s, 1H, NH), 4.18 (m, |
| | 2H), 2.73 (s, 3H), 1.45 (s, 9H). |
| 202 | δ 8.00 (s, 1H), 7.84 (d, 1H), 7.80 (s, 1H, NH), 7.60 (d, |
| | 1H), 7.36 (t, 1H), 6.46 (br s, 1H, NH), 5.88 m, 1H), 5.24 |
| | (m, 2H), 4.06 (t, 2H), 2.73 (s, 3H), 1.45 (s, 9H). |
| 203 | δ 8.00 (s, 1H), 7.84 (d, 1H), 7.80 (s, 1H, NH), 7.60 (d, |
| | 1H), 7.36 (t, 1H), 6.46 (br s, 1H, NH), 4.26 (m, 2H), 2.73 |
| | (s, 3H), 1.45 (s, 9H). |
| 204 | δ 8.00 (s, 1H), 7.84 (d, 1H), 7.60 (s, 1H, NH), 7.46 (d, |
| | 1H), 7.40 (t, 1H), 4.20 (q, 2H), 3.50 (q, 2H), 1.45 (s, 9H), |
| | 1.20 (m, 6H). |
| 205 | δ 8.20 (s, 1H, NH), 7.64 (dd, 1H), 7.52 (d, 1H), 7.32 (t, |
| | 1H), 7.02 (dd, 1H), 3.50 (m, 2H), 3.28 (m, 2H), 3.18 (q, |
| | 2H), 1.42 (s, 9H), 1.22 (m, 9H). |
| 206 | δ 8.00 (s, 1H), 7.84 (d, 1H), 7.60 (s, 1H, NH), 7.56 (d, |
| | 1H), 7.40 (t, 1H), 6.56 (br s, 1H, NH), 4.08 (m, 2H), 3.20 |
| | (q, 2H), 1.45 (s, 9H), 1.20 (t, 3H). |
| 207 | δ 8.00 (s, 1H), 7.86 (s, 1H, NH), 7.80 (dd, 1H), 7.52 (d, |
| | 1H), 7.42 (t, 1H), 6.40 (t, 1H, NH), 5.92 (m, 1H), 5.24 (m, |
| | 2H), 4.04 (m, 2H), 3.18 (q, 2H), 1.42 (s, 9H), 1.22 (t, 3H). |
| 209 | δ 8.00 (s, 1H,), 7.80 (dd, 1H), 7.66 (s, 1H, NH), 7.62 (d, |
| | 1H,), 7.42 (t, 1H), 6.60 (t, 1H, NH), 4.64 (m, 1H), 4.52 (m, |
| | 1H), 3.80 (m, 2H), 3.04 (q, 2H), 1.42 (s, 9H), 1.22 (t, 3H). |
| 210 | δ 8.00 (s, 1H,), 7.80 (dd, 1H), 7.66 (s, 1H, NH), 7.62 (d, |
| | 1H,), 7.42 (t, 1H), 6.20 (t, 1H, NH), 3.50 (q, 2H), 3.04 (q, |
| | 2H), 1.42 (s, 9H), 1.22 (t, 3H). |
| 211 | δ 8.00 (s, 1H,), 7.90 (s, 1H, NH), 7.80 (dd, 1H), 7.62 (d, |
| | 1H,), 7.42 (t, 1H), 6.40 (t, 1H, NH), 3.20 (m, 2H), 3.04 (q, |
| | 2H), 1.80 (m, 1H), 1.42 (s, 9H), 1.22 (t, 3H). |
| 212 | δ 8.00 (s, 1H,), 7.90 (s, 1H, NH), 7.80 (dd, 1H), 7.62 (d, |
| | 1H,), 7.40 (t, 1H), 6.40 (t, 1H, NH), 3.20 (m, 2H), 3.04 (q, |
| | 2H), 1.35 (s, 9H), 1.22 (t, 3H), 1.00 (m, 1H), 0.58 (m, 2H), |
| | 0.30 (m, 2H). |
| 213 | δ 8.40 (d, 1H), 8.04 (s, 1H, NH), 8.00 (d, 1H), 7.82 (t, |
| | 1H), 7.60 (t 1H, NH), 3.54 (q, 2H), 3.06 (q, 2H), 1.42 (s, |
| | 9H), 1.32 (m, 6H). |
| 214 | δ 8.34 (d, 1H), 8.04 (s, 1H, NH), 7.82 (t, 1H), 7.22 (d, |
| | 1H), 3.54 (q, 2H), 3.36 (q, 2H), 3.06 (q, 2H), 1.42 (s, 9H), |
| | 1.32 (m, 9H). |
| 215 | δ 8.40 (d, 1H), 8.04 (s, 1H, NH), 8.00 (m, 2H), 3.34 (q, |
| | 2H), 3.06 (q, 2H), 1.42 (s, 9H), 1.32 (t, 3H), 1.00 (m, 1H), |
| | 0.56 (m, 2H), 0.36 (m, 2H). |
| 216 | δ 8.40 (d, 1H), 8.04 (s, 1H, NH), 7.96 (d, 1H), 7.84 (t, |
| | 1H), 7.60 (br s, 1H, NH), 3.30 (t, 2H), 3.06 (q, 2H), 1.90 |
| | (m, 1H), 1.42 (s, 9H), 1.32 (t, 3H), 1.00 (d, 6H). |
| 217 | δ 8.40 (d, 1H), 8.04 (s, 1H, NH), 7.96 (m, 3H), NH & |
| | aromatic), 4.22 (m, 2H), 3.06 (q, 2H), 2.22 (m, 1H), 1.42 |
| | (s, 9H), 1.32 (t, 3H). |
| 218 | δ 8.8 (br s, 1H), 7.75 (d, 1H), 7.70 (s, 1H), 7.59 (s, 1H), |
| | 7.38 (t, 1H), 7.11 (d, 1H), 3.5 (m, 2H), 3.3 (m, 2H), 1.6 (s, |
| | 9H), 1.3 (m, 3H), 1.1 (m, 3H). |
| 219 | δ 8.77 (br s, 1H), 8.18 (s, 1H), 7.81 (d, 1H), 7.6 (s, 1H), |
| | 7.56 (d, 1H), 7.42 (t, 1H), 6.27 (br s, 1H), 3.5 (m, 2H), 1.6 |
| | (s, 9H), 1.26 (t, 3H). |
| 220 | δ 8.74 (br s, 1H), 7.78 (d, 1H), 7.70 (s, 1H), 7.6 (s, 1H), |
| | 7.6 (s, 1H), 7.37 (t, 1H), 7.1 (d, 1H), 3.5 (m, 2H), 3.3 (m, |
| | 2H), 1.6 (s, 9H), 1.3 (m, 3H), 1.1 (m, 3H). |
| 225 | δ 8.42 (dd, 1H), 7.74 (s, 1H), 7.19 (m, 2H), 3.12 (q, 3H), |
| | 3.03 (s, 3H), 3.00 (s, 3H), 1.37 (s, 9H), 1.30 (t, 6H). |
| 226 | δ 8.70 (dd, 1H), 7.74 (s, 1H), 7.70 (m, 1H,), 7.20 (t, 1H), |
| | 3.42 q, 2H), 3.08 (q, 2H), 1.28 (s, 9H), 1.26 (t, 3H), 1.20 |
| | (t, 3H). |
| 227 | δ 8.45 (s, 1H), 7.64 (dd, 1H), 7.58 (s, 1H), 7.36 (t, 1H), |
| | 7.08 (d, 1H), 3.20 (m, 2H), 2.99 (s, 3H), 2.70 (s, 3H), 1.44 |
| | (s, 9H), 1.05 (t, 3H). |
| 228 | δ 8.34 (s, 1H), 7.74 (d, 1H), 7.60 (s, 1H), 7.34 (t, 1H), 7.16 |
| | (s, 1H), 3.12 (q, 2H), 3.06 (s, 3H), 3.00 (s, 3H), 1.42 (s, |
| | 9H), 1.25 (t, 3H). |
| 229 | δ 8.20 (s, 1H,), 7.68 (dd, 1H), 7.60 (s, 1H,), 7.32 (t, 1H), |
| | 7.02 (dd, 1H), 3.32 (m, 2H), 3.28 (m, 2H), 3.10 (s, 3H), |
| | 1.45 (s, 9H), 1.22 (m, 6H). |
| 230 | δ 8.40 (d, 1H), 7.80 (d, 1H), 7.22 (dd, 1H), 3.50 (m, 4H), |
| | 3.18 (q, 2H), 1.42 (s, 9H), 1.22 (m, 6H). |
| 231 | δ 8.40 (d, 1H), 7.74 (s, 1H), 7.18 (m, 2H), 3.28 (m, 2H), |
| | 3.08 (q, 2H), 3.02 (s, 3H), 1.45 (s, 9H), 1.32 (t, 3H), 1.26 |
| | (t, 3H). |
| 237 | δ 8.80 (dd, 1H), 7.72 (m, 1H,), 7.20 (m, 1H), 6.36 (s, 1H), |
| | 5.86 (m, 1H,), 5.22 (m, 2H), 4.10 (m, 2H), 3.08 (q, 2H), |
| | 1.35 (s, 9H), 1.32 (t, 3H). |
| 238 | δ 1.3 (t, 3H), 1.4 (t, 3H), 1.6 (s, 9H), 3.0 (q, 2H), 4.4 (q, |
| | 2H), 7.6 (d, 1H), 8.0 (s, 1H), 8.3 (d, 1H), 8.6 (br s, 1H, |
| | NH), 8.8 (s, 1H). |
| 242 | δ 8.6 (dd, 1H), 7.95 (br s, 1H, NH), 7.3 (m, 2H), 7.2 (m, |
| | 1H), 6.48 (s, 1H), 4.6 (m, 2H), 3.63 (m, 2H), 3.49 (m, 2H), |
| | 1.95 (m, 4H), 1.44 (t, 3H), 1.34 (s, 9H). |
| 243 | δ 8.5 (dd, 1H), 7.9 (br s, 1H, NH), 7.18 (m, 2H), 6.48 (s, |
| | 1H), 4.57 (m, 2H), 3.7 (m, 8H), 1.44 (t, 3H), 1.33 (s, 9H). |
| 244 | δ 8.75 (dd, 1H), 7.8 (br s, 1H, NH), 7.45 (m, 1H), 7.2 (m, |
| | 1H), 6.49 (s, 1H), 4.59 (m, 2H), 3.63 (s, 3H), 3.36 (s, 3H), |
| | 1.42 (t, 3H), 1.26 (s, 9H). |
| 245 | δ 8.35 (d, 1H), 8.33 (br s, 1H, NH), 7.8 (t, 3H), 7.43 (d, |
| | 1H), 6.54 (s, 1H), 4.6 (q, 2H), 3.62 (m, 4H), 2.00 (m, 4H), |
| | 1.4 (t, 3H), 1.29 (s, 9H). |
| 246 | δ 8.4 (d, 1H), 8.37 (br s, 1H, NH), 7.8 (t, 1H), 7.4 (d, 1H), |
| | 6.55 (s, 1H), 4.6 (q, 2H), 3.66 (s, 3H), 3.4 (s, 3H), 1.5 (t, |
| | 3H), 1.31 (s, 9H). |
| 247 | δ 8.4 (d, 1H), 8.39 (br s, 1H, NH), 7.85 (t, 1H), 7.29 (d, |
| | 1H), 6.55 (s, 1H), 4.6 (q, 2H), 3.3-4.00 (m, 8H), 1.33 (t, |
| | 3H), 1.26 (s, 9H). |
| 248 | δ 8.88 (t, 1H), 7.65 (s, 1H), 6.98 (t, 1H), 6.48 (s, 1H), 5.22 |
| | (m, 1H), 4.57 (q, 2H), 1.46 (t, 2H), 1.40 (d, 6H), 1.33 (s, |
| | 9H). |
| 249 | δ 8.83 (t, 1H), 7.65 (s, 1H), 6.99 (t, 1H), 6.48 (s, 1H), 4.56 |
| | (q, 2H), 4.30 (t, 2H), 1.80 (m, 2H), 1.47 (t, 2H), 1.33 (s, |
| | 9H), 1.03 (t, 3H). |
| 250 | δ 8.83 (t, 1H), 7.64 (s, 1H), 6.98 (t, 1H), 6.45 (s, 1H), 4.58 |
| | (q, 2H), 4.40 (q, 2H), 1.45 (t, 3H), 1.41 (t, 2H), 1.33 (s, |
| | 9H). |
| 251 | δ 8.83 (m, 1H), 7.62 (s, 1H), 6.98 (t, 1H), 6.55 (br s, 1H), |
| | 6.45 (s, 1H), 4.56 (q, 2H), 3.03 (d, 3H), 1.43 (t, 3H), 1.33 |
| | (s, 9H). |
| 252 | δ 8.83 (m, 1H), 7.62 (s, 1H), 6.98 (t, 1H), 6.55 (br s, 1H), |
| | 6.49 (s, 1H), 4.56 (q, 2H), 3.52 (m, 2H), 1.43 (t, 3H), 1.33 |
| | (s, 9H), 1.25 (t, 3H). |
| 253 | δ 8.23 (m, 1H), 7.92 (s, 1H), 6.97 (t, 1H), 6.53 (s, 1H), |
| | 4.56 (q, 2H), 3.55 (q, 2H), 3.22 (q, 2H), 1.43 (t, 3H), 1.33 |
| | (s, 9H), 1.27 (t, 3H), 1.14 (t, 3H). |
| 254 | δ 8.23 (m, 1H), 8.02 (s, 1H), 6.97 (m, 1H), 6.53 (s, 1H), |
| | 4.54 (q, 2H), 3.50 and 3.35 (q, 2H), amide isomers), 3.09 |
| | and 2.93 (s, 3H, amide isomers), 1.43 (t, 3H), 1.33 (s, 9H), |
| | 1.25 and 1.16 (t, 3H, amide isomers). |
| 256 | δ 8.82 (s, 1H), 8.52 (d, 1H), 8.00 (m, 1H), 7.72 (s, 1H), |
| | 6.66 (s, 1H), 4.62 (q, 2H), 3.10 (s, 3H), 3.09 (s, 3H), 1.42 |
| | (t, 3H), 1.33 (s, 9H). |
| 257 | δ 8.78 (s, 1H), 8.42 (d, 1H), 8.10 (m, 1H), 7.82 (s, 1H), |
| | 7.72 (s, 1H), 6.66 (s, 1H), 4.62 (q, 2H), 3.82 (q, 2H), 1.42 |
| | (t, 3H), 1.33 (s, 9H), 1.30 (t, 3H). |
| 258 | δ 8.72 (s, 1H), 8.41 (s, 1H), 8.26 (s, 1H), 8.12 (s, 1H), |
| | 6.59 (s, 1H), 4.57 (q, 2H), 3.14 (s, 3H), 3.05 (s, 3H), 1.45 |
| | (t, 3H), 1.34 (s, 9H). |
| 259 | δ 8.90 (s, 1H), 8.78 (s, 1H), 8.50 (s, 1H), 8.04 (s, 1H), |
| | 6.56 (s, 1H), 6.20 (s, 1H), 4.58 (q, 2H), 3.52 (q, 2H), 1.48 |
| | (t, 3H), 1.32 (s, 9H), 1.26 (t, 3H). |
| 260 | δ 8.70 (s, 1H), 8.58 (s, 1H), 8.40 (s, 1H), 7.80 (s, 1H), |
| | 6.54 (s, 1H), 6.0 (m, 1H), 5.80 (m, 1H), 4.57 (q, 2H), 4.51 |
| | (m, 2H), 4.30 (m, 2H), 1.47 (t, 3H), 1.34 (s, 9H). |
| 262 | δ 10.50 (s, 1H), 9.00 (s, 1H,), 8.40 (s, 1H), 8.20 (s, 1H), |
| | 6.96 (s, 1H,), 5.92 (m, 2H), 5.30 (m, 4H), 4.32 (q, 2H), |
| | 4.12 (m, 2H), 3.80 (m, 2H), 1.32 (t, 3H), 1.30 (s, 9H). |
| 263 | δ 8.70 (s, 1H,), 8.42 (s, 1H), 8.18 (m, 1H,), 8.08 (s, 1H), |
| | 6.58 (s, 1H), 4.57 (q, 2H), 3.60 (q, 2H), 3.10 (s, 3H), 1.46 |
| | (t, 3H), 1.34 (s, 9H), 1.26 (m, 3H). |
| 264 | δ 8.68 (s, 1H), 8.41 (s, 1H), 8.26 (s, 1H), 8.12 (s, 1H), 6.59 |
| | (s, 1H), 4.57 (q, 2H), 3.86 (s, 2H), 2.12 (m, 1H), 1.45 (t, |
| | 3H), 1.34 (s, 9H). |
| 265 | δ 8.78 (d, 1H,), 8.12 (d, 1H,), 6.47 (s, 1H), 4.42 (q, 2H), |
| | 3.82 (s, 3H), 3.76 (s, 3H), 1.36 (t, 3H), 1.29 (s, 9H). |
| 266 | δ 8.80 (d, 1H,), 8.08 (s, 1H), 8.02 (d, 1H,), 6.47 (s, 1H), |
| | 4.42 (q, 2H), 4.02 (q, 2H), 1.36 (m, 6H), 1.29 (s, 9H). |
| 269 | δ 9.3 (s, 1H), 8.70 (d, 1H), 8.65 (m, 1H), 7.15 (t, 1H), 6.20 |
| | (br s, 1H), 4.12 (m, 1H), 3.70 (t, 1H), 3.50 (m, 2H), 3.30 |
| | (dd, 1H), 3.15 (m, 1H), 2.95 (m, 1H), 2.80 (dd, 1H), 1.34- |
| | 1.20 (m, 6H), 1.17 (s, 9H). |
| 270 | δ 8.37 (t, 1H), 7.90 (s, 1H), 6.93 (t, 1H), 6.52 (s, 1H), 4.55 |
| | (q, 2H), 3.12 (s, 3H), 2.97 (s, 3H), 1.43 (t, 3H), 1.33 (s, |
| | 9H). |
| 271 | δ 8.90 and 8.75 (d, 1H), amide isomers), 8.18 and 8.13 (s, |
| | 1H), amide isomers), 7.88 and 7.70 (m, 1H), 7.22 and 7.19 |
| | (t, 1H), amide isomers), 4.58 (q, 2H), 1.45 (t, 3H), 1.34 |
| | (s, 9H). |
| 272 | δ 7.82 (s, 1H), 7.60 (s, 1H), 7.45 (d, 1H), 7.36 (t, 1H), 7.00 |
| | (d, 1H), 6.50 (s, 1H), 4.55 (q, 2H), 1.43 (t, 3H), 1.32 (s, |
| | 9H). |
| 273 | δ 7.65 (s, 1H), 7.28 (s, 1H), 7.21 (t, 1H), 7.02 (d, 1H), 7.72 |
| | (d, 1H), 6.45 (s, 1H), 4.45 (m, 3H), 1.42 (t, 3H), 1.33 (d, |
| | 6H), 1.32 (s, 9H). |
| 274 | δ 8.8 (br m 1H, NH), 8.3 (d, 1H), 7.8 (m, 1H), 7.4 (d, 1H), |
| | 4.6 (q, 2H), 3.14 (s, 3H), 3.06 (s, 3H), 1.42 (t, 3H), 1,36 (s, |
| | 9H). |
| 275 | δ 8.8 (dd, 1H), 8.33 (br m, 1H, NH), 7.7 (m, 1H), 7.2 (m, |
| | 1H), 6.2 (br s, 1H), 4.6 (q, 2H), 3.5 (q, 2H), 1.37 (t, 3H), |
| | 1.29 (s, 9H), 1.24 (t, 3H). |
| 276 | δ 8.26 (dd, 1H), 8.3 (br m, 1H, NH), 7.2 (br s, 1H), 7.17 |
| | (m, 1H), 4.55 (q, 2H), 3.1 (s, 3H), 3.03 (s, 3H), 1.43 (t, |
| | 3H), 1.37 (s, 9H). |
| 277 | δ 8.78 (s, 1H), 8.52 (m, 1H), 7.60 (t, 1H), 6.52 (s, 1H), |
| | 4.59 (q, 2H), 3.42 (q, 2H), 1.37 (t, 3H), 1.34 (s, 9H), 1.28 |
| | (t, 3H). |
| 278 | δ 8.46 (dd, 1H), 8.32 (s, 1H), 7.62 (t, 1H), 6.60 (s, 1H), |
| | 4.59 (q, 2H), 4.28 (d, 2H), 2.30 (t, 1H), 1.42 (t, 3H), 1.34 |
| | (s, 9H). |
| 279 | δ 8.40 (s, 1H), 8.36 (m, 1H), 7.42 (t, 1H), 6.46 (s, 1H), |
| | 4.50 (q, 2H), 3.28 (q, 2H), 3.13 (s, 3H), 1.42 (t, 3H), 1.34 |
| | (s, 9H), 1.30 (t, 3H). |
| 280 | δ 8.40 (s, 1H), 8.38 (dd, 1H), 7.60 (t, 1H), 6.53 (s, 1H), |
| | 5.90 (m, 1H), 5.80 (m, 1H), 4.56 (q, 2H), 4.46 (m, 2H), |
| | 4.20 (m, 2H), 1.42 (t, 3H), 1.34 (s, 9H). |
| 281 | δ 9.24 (s, 1H), 8.58 (d, 1H), 8.22 (dd, 1H), 8.00 (d, 1H), |
| | 6.68 (s, 1H), 5.81 (m, 2H), 4.60 (m, 2H), 4.46 (m, 2H), |
| | 4.28 (m, 2H), 1.46 (t, 3H), 1.30 (s, 9H). |
| 282 | δ 8.56 (d, 1H), 8.32 (dd, 1H), 8.22 (t, 1H), 8.00 (d, 1H), |
| | 6.63 (s, 1H), 4.59 (q, 2H), 4.28 (d, 2H), 2.30 (t, 1H), 1.42 |
| | (t, 3H), 1.34 (s, 9H). |
| 283 | δ 8.51 (d, 1H), 8.42 (t, 1H), 8.38 (s, 1H), 8.22 (dd, 1H), |
| | 6.63 (s, 1H), 4.59 (q, 2H), 4.00 (m, 2H), 1.45 (t, 3H), 1.36 |
| | (s, 9H). |
| 284 | δ 9.00 (s, 1H), 8.48 (d, 1H), 8.40 (t, 1H), 8.36 (dd, 1H), |
| | 8.00 (s, 1H), 6.74 (s, 1H), 4.58 (m, 2H), 4.40 (t, 2H), 3.60 |
| | (m, 2H), 1.46 (t, 3H), 1.32 (s, 9H). |
| 285 | δ 8.80 (s, 1H), 7.84 (dd, 1H), 7.60 (s, 1H), 7.32 (m, 1H), |
| | 7.02 (m, 1H), 6.53 (s, 1H), 5.82 (m, 1H), 5.20 (m, 2H), |
| | 4.56 (q, 2H), 4.20 (m, 2H), 3.00 (s, 3H), 1.42 (t, 3H), 1.24 |
| | (s, 9H). |
| 286 | δ 8.40 (s, 1H), 7.68 (m, 2H), 7.40 (m, 2H), 6.53 (s, 1H), |
| | 5.80 (m, 2H), 4.56 (q, 2H), 4.40 (m, 2H), 4.30 (m, 2H), |
| | 1.42 (t, 3H), 1.24 (s, 9H). |
| 287 | δ 9.60 (s, 1H), 8.60 (s, 1H), 8.40 (s, 1H), 6.59 (s, 1H), |
| | 4.58 (m, 2H), 3.16 (s, 3H), 3.08 (s, 3H), 1.44 (t, 3H), 1.32 |
| | (s, 9H). |
| 288 | δ 9.80 (s, 1H), 9.20 (s, 1H), 9.00 (s, 1H), 8.20 (s, 1H), |
| | 6.59 (s, 1H), 4.58 (m, 2H), 3.36 (q, 2H), 1.34 (t, 3H), 1.32 |
| | (s, 9H), 1.26 (t, 3H). |
| 289 | δ 9.78 (s, 1H), 9.18 (s, 1H), 8.40 (s, 1H), 7.72 (s, 1H), 6.64 |
| | (s, 1H), 4.58 (m, 2H), 4.26 (q, 2H), 2.24 (t, 1H), 1.34 (t, |
| | 3H), 1.32 (s, 9H). |
| 290 | δ 9.68 (s, 1H), 8.80 (s, 1H), 8.20 (s, 1H), 7.72 (s, 1H), 6.56 |
| | (s, 1H), 5.88 (m, 2H), 4.56 (q, 2H), 4.48 (m, 4H), 1.34 (t, |
| | 3H), 1.32 (s, 9H). |
| 291 | δ 9.82 (s, 1H), 9.20 (s, 1H), 8.40 (s, 1H), 7.82 (t, 1H), 6.66 |
| | (s, 1H), 4.58 (m, 2H), 4.06 (m, 2H), 1.34 (t, 3H), 1.32 (s, |
| | 9H), 1.26 (t, 3H). |
| 294 | δ 8.4 (d, 1H), 7.85 (br s, 1H), 7.1 (m, 2H), 6.5 (s, 1H), 4.5 |
| | (q, 2H), 3.6 (s, 3H), 3.2 (s, 3H), 1.4 (t, 3H), 1.3 (s, 9H). |
| 295 | δ 8.40 (s, 1H), 8.16 (s, 1H), 8.02 (d, 1H), 7.80 (dd, 1H), |
| | 7.40 (t, 1H), 6.56 (s, 1H), 4.54 (q, 2H), 4.40 (m, 2H), 1.45 |
| | (s, 3H), 1.42 (m, 6H), 1.00 (m, 2H), 0.80 (m, 2H). |
| 296 | δ 9.00 (dd, 1H), 7.80 (m, 1H), 7.76 (s, 1H), 7.20 (t, 1H), |
| | 6.42 (s, 1H), 4.54 (q, 2H), 4.40 (m, 2H), 1.45 (s, 3H), 1.42 |
| | (m, 6H), 1.00 (m, 2H), 0.80 (m, 2H). |
| 297 | δ 8.40 (s, 1H), 8.20 (d, 1H), 6.80 (d, 1H), 6.60 (dd, 1H), |
| | 6.42 (s, 2H), 4.56 (q, 2H), 3.92 (s, 3H), 3.80 (s, 3H), 1.42 |
| | (t, 3H), 1.24 (s, 9H). |
| 298 | δ 7.80 (s, 1H), 7.60 (d, 1H), 7.00 (d, 1H), 6.80 (dd, 1H), |
| | 6.42 (s, 2H), 4.56 (q, 2H), 3.82 (s, 6H), 1.42 (t, 3H), 1.24 |
| | (s, 9H). |
| 299 | δ 8.40 (dd, 1H), 7.90 (s, 1H), 7.20 (m, 2H), 6.42 (s, 1H), |
| | 4.54 (q, 2H), 3.10 (s, 3H), 3.08 (s, 3H), 1.45 (s, 3H), 1.42 |
| | (m, 6H), 1.00 (m, 2H), 0.84 (m, 2H). |
| 300 | δ 8.48 (d, 1H), 8.00 (s, 1H), 7.90 (s, 1H), 7.62 (m, 2H), |
| | 6.42 (s, 1H), 4.54 (q, 2H), 3.40 (q, 2H), 1.45 (s, 3H), 1.42 |
| | (t, 3H), 1.20 (t, 3H), 1.00 (m, 2H), 0.84 (m, 2H). |
| 301 | δ 8.60 (dd, 1H), 7.90 (s, 1H), 7.20 (m, 2H), 6.42 (s, 1H), |
| | 5.84 (m, 2H), 4.51 (q, 2H), 4.30 (m, 4H), 1.45 (s, 3H), |
| | 1.42 (m, 6H), 1.00 (m, 2H), 0.84 (m, 2H). |
| 302 | δ 8.80 (m, 1H), 7.90 (s, 1H), 7.60 (m, 1H), 7.20 (m, 1H), |
| | 6.60 (s, 1H), 6.42 (s, 1H), 4.50 (q, 2H), 4.20 (m, 2H), 2.30 |
| | (m, 1H), 1.45 (s, 3H), 1.42 (m, 6H), 1.00 (m, 2H), 0.84 (m, |
| | 2H). |
| 303 | δ 8.20 (s, 1H), 7.70 (d, 1H), 7.60 (s, 2H), 7.40 (t, 1H), 7.20 |
| | (d, 1H), 6.42 (s, 1H), 4.54 (q, 2H), 3.10 (s, 3H), 3.00 (s, |
| | 3H), 1.45 (s, 3H), 1.42 (m, 6H), 1.00 (m, 2H), 0.84 (m, |
| | 2H). |
| 304 | δ 7.90 (s, 1H), 7.82 (s, 1H), 7.62 (dd, 1H), 7.40 (t, 1H), |
| | 7.30 (d, 1H), 6.42 (s, 1H), 5.80 (m, 2H), 4.54 (q, 2H), |
| | 4.40 (m, 4H), 1.45 (s, 3H), 1.42 (m, 6H), 1.00 (m, 2H), |
| | 0.84 (m, 2H). |
| 306 | δ 8.08 (s, 1H), 8.00 (s, 1H), 7.82 (dd, 1H), 7.40 (m, 2H), |
| | 6.42 (s, 1H), 6.20 (br s, 1H), 4.54 (q, 2H), 3.44 (q, 2H), |
| | 1.45 (s, 3H), 1.42 (m, 6H), 1.00 (m, 2H), 0.84 (m, 2H). |
| 307 | δ 9.32 (s, 1H), 8.58 (d, 1H), 7.15 (m, 2H), 3.68 (t, 1H), |
| | 3.57 and 3.33 (br m, 2H), 3.32 (dd, 1H), 3.18 (dq, 1H), |
| | 3.50 and 2.95 (br s, 3H), amide isomers), 2.94 (dq, 1H), |
| | 2.81 (d d, 1H), 1.23 (t, 3H), 1.17 (s, 9H). |
| 308 | δ 9.32 (s, 1H), 8.42 (d, 1H), 7.12 (d, 2H), 3.68 (t, 1H), |
| | 3.5 (br s, 2H), 3.3 (br s, 2H), 3.3 (dd, 1H), 3.18 (m, 1H), |
| | 2.95 (m, 1H), 2.80 (dd, 1H), 1.34-1.17 (m, 18H). |
| 309 | δ 9.32 (s, 1H), 8.55 (d, 1H), 7.28 (m, 1H), 7.13 (dd, 1H), |
| | 3.68 (t, 1H), 3.62 (br m, 2H), 3.48 (br m, 2H), 3.31 (dd, |
| | 1H), 3.17 (dq, 1H), 2.95 (dq, 1H), 2.81 (dd, 1H), 1.9 (m, |
| | 4H), 1.23 (t, 3H), 1.17 (s, 9H). |
| 310 | δ 9.32 (s, 1H), 8.58 (d, 1H), 7.28 (m, 1H), 7.17 (dd, 1H), |
| | 5.92 (br s, 1H), 5.75 (br s, 1H), 4.43 (br s, 2H), 4.29 (br s, |
| | 2H), 3.69 (t, 1H), 3.31 (dd, 1H), 3.17 (dq, 1H), 2.95 (dq, |
| | 1H), 2.81 (dd, 1H), 1.23 (t, 3H), 1.17 (s, 9H). |
| 311 | δ 7.7 (m, 1H), 7.6 (m, 1H), 7.33-7.36 (m, 3H), 2.2 (s, 3H), |
| | 1.3 (s, 9H). |
| 312 | δ 8.70 (s, 1H, NH), 8.42 (m, 1H), 7.82 (d, 2H), 6.64 (s, |
| | 1H), 4.12 (s, 3H), 4.01 (s, 3H), 1.31 (s, 9H). |
| 313 | δ 8.42 (d, 1H), 7.92 (m, 2H), 7.80 (s, 1H, NH), 6.60 (s, |
| | 1H), 4.04 (s, 3H), 3.40 (q, 2H), 1.31 (s, 9H). |
| 314 | δ 8.50 (s, 1H, NH), 8.22 (d, 1H), 7.80 (t, 1H), 7.30 (d, |
| | 1H), 6.60 (s, 1H), 4.04 (s, 3H), 3.56 (q, 2H), 3.32 (q, 2H), |
| | 1.43 (s, 9H), 1.30 (t, 3H), 1.15 (t, 3H). |
| 315 | δ 8.39 (d, 1H), 8.20 (s, 1H, NH), 7.92 (d, 1H), 7.84 (t, |
| | 1H), 7.80 (s, 1H), 6.60 (s, 1H), 4.02 (s, 3H), 2.96 (m, 1H), |
| | 1.31 (s, 9H), 0.88 (m, 2H), 0.68 (m, 2H). |
| 316 | δ 8.42 (m, 2H), 7.92 (m, 1H, NH), 7.80 (s, 1H), 6.60 (s, |
| | 1H), 4.12 (m, 2H), 4.00 (s, 3H), 2.22 (m, 1H), 1.31 (s, 9H). |
| 317 | δ 8.42 (m, 2H), 7.92 (m, 1H, NH), 7.80 (s, 1H), 6.60 (s, |
| | 1H), 4.12 (m, 2H), 4.00 (s, 3H), 2.22 (m, 1H), 1.31 (s, 9H). |
| 318 | δ 8.46 (m, 2H, NH and 1 aromatic), 7.80 (m, 1H), 7.32 |
| | (dd, 1H), 6.55 (s, 1H), 5.82 (m, 1H), 5.34 (m, 2H), 4.20 (d, |
| | 2H), 4.02 (s, 3H), 1.30 (s, 9H). |
| 319 | δ 8.34 (s, 1H), 7.74 (d, 1H), 7.60 (s, 1H), 7.34 (t, 1H), |
| | 7.16 (s, 1H), 4.10 (m, 2H), 3.12 (q, 2H), 2.20 (m, 1H), |
| | 1.42 (s, 9H), 1.25 (t, 3H). |
| 326 | δ 8.45 (s, 1H), 7.72 (dd, 1H), 7.60 (s, 1H), 7.32 (t, 1H), |
| | 7.08 (d, 1H), 3.05 (s, 3H), 2.99 (s, 3H), 2.70 (s, 3H), 1.44 |
| | (s, 9H). |
| 327 | δ 8.80 (dd, 1H), 7.72 (m, 1H,), 7.20 (m, 1H), 5.86 (m, |
| | 2H,), 5.22 (m, 4H), 4.10 (m, 4H), 3.08 (q, 2H), 1.35 (s, |
| | 9H), 1.32 (t, 3H). |
| 329 | δ 7.42 (m, 3H), 7.10 (dd, 1H), 6.80 (s, 1H), 6.52 (s, 1H, |
| | NH), 4.50 (q, 2H), 4.20 (t, 2H), 2.28 (t, 2H), 2.20 (q, 2H), |
| | 1.42 (m, 6H), 1.38 (s, 9H). |
| 331 | δ 9.36 (br s, 1H), 8.42 (dd, 1H), 7.27 (m, 2H), 3.68 (t, 1H), |
| | 3.68 (br s, 2H), 3.38 (br s, 2H), 3.30 (dd, 1H), 3.18 (d of q, |
| | 1H), 2.95 (d of q, 1H), 2.80 (dd, 1H), 1.58-1.70 (m, 6H), |
| | 1.25 (t, 3H), 1.17 (s, 9H). |
| 332 | δ 7.62 (br s, 1H), 7.54 (br s, 1H), 7.36 (m, 2H), 6.90 (m, |
| | 1H), 6.47 (s, 1H), 4.56 (q, 2H), 1.45 (t, 3H), 1.32 (s, 9H). |
| 333 | δ 7.70 (br s, 1H), 7.60 (br s, 1H), 7.44 (d, 1H), 7.39 (t, |
| | 1H), 7.02 (d, 1H), 6.47 (s, 1H), 4.56 (q, 2H), 1.45 (t, 3H), |
| | 1.31 (s, 9H). |
| 335 | δ 9.38 (br s, 1H), 8.93 (dd, 1H), 7.67 (m, 1H), 7.13 (dd, |
| | 1H), 4.36 (q, 2H), 3.64 (dd, 1H), 3.53 (dd, 1H), 3.23 (t, |
| | 1H), 2.41 (m, 2H), 1.38 (t, 3H), 1.25 (s, 9H), 1.17 (t, 3H). |
| 338 | δ 8.41 (m, 1H), 7.83 (br s, 1H), 7.16 (dd, 1H), 6.92 (m, |
| | 2H), 7.47 (s, 1H), 4.56 (q, 2H), 1.45 (t, 3H), 1.32 (s, 9H). |
| 340 | δ 9.38 (br s, 1H), 8.47 (dd, 1H), 7.26 (m, 1H), 7.13 (dd, |
| | 1H), 5.90 (m, 1H), 5.73 (m, 1H), 4.43 (m, 2H), 4.32 (m, |
| | 2H), 3.64 (dd, 1H), 3.53 (dd, 1H), 3.24 (t, 1H), 2.41 (m, |
| | 2H), 1.25 (s, 9H), 1.17 (t, 3H). |
| 341 | δ 9.38 (br s, 1H), 8.45 (dd, 1H), 7.26 (m, 1H), 7.13 (dd, |
| | 1H), 3.62 (m, 3H), 3.43 (m, 3H), 3.23 (t, 1H), 2.41 (m, |
| | 2H), 1.90 (m, 2H), 1.23 (s, 9H), 1.16 (t, 3H). |
| 342 | δ 9.38 (br s, 1H); 8.38 (d, 1H), 7.13 (m, 2H); 3.62 (dd, |
| | 1H); 3.58 (br s, 1H); 3.53 (dd; 1H); 3.25 (br s, 1H); 3.23 |
| | (t, 1H); 3.02, 2.98 (br s, 3H); 2.41 (m, 2H); 1.23 (s, 9H); |
| | 1.16 (t, 3H); 1.14 (t, 3H). |
| 343 | δ 9.38 (br s, 1H); 8.38 (dd, 1H), 7.13 (m, 2H); 3.62 (dd, |
| | 1H); 3.52 (d of d; 1H); 3.23 (t, 1H); 3.09 (br s, 3H), 2.99 |
| | (br s,3H; 2.41 (m, 2H; 1.23 (s, 9H); 1.16 (t, 3H). |
| 346 | δ 9.0 (br s 2H), 8.0 (m, 1H), 7.25 (m, 1H), 6.4 (s, 1H), 4.6 |
| | (q, 2H), 4.4 (q, 2H), 1.45 (t, 3H), 1.4 (t, 3H), 1.3 (s, 9H). |
| 347 | δ 8.56 (dd, 1H), 8.00 (br s, 1H, NH), 7.24 (m, 2H), 6.54 (s, |
| | 1H), 4.57 (q, 2H), 3.92 (s, 2H), 1.42 (t, 3H), 1.28 (s, 9H). |
| 348 | δ 8.14 (m, 1H), 8.00 (dd, 1H), 7.26 (s, 1H), 6.26 (s, 1H), |
| | 4.54 (q, 2H), 4.12 (q, 2H), 1.41 (m, 6H), 1.20 (s, 9H). |
| 349 | δ 8.65 (dd, 1H), 7.85 (br s, 1H), 7.65 (m, 1H), 7.6 (br s, |
| | 1H), 7.15 (dd, 1H), 6.5 (s, 1H), 4.6 (q, 2H), 3.85 (m, 2H), |
| | 1.4 (m, 3H), 1.3 (m, 3H), 1.25. |
| 350 | δ 8.56 (dd, 1H), 8.00 (br s, 1H, NH), 7.24 (m, 2H), 6.54 (s, |
| | 1H), 4.57 (q, 2H), 3.92 (m, 2H), 3.10 (m, 2H), 1.42 (t, 3H), |
| | 1.28 (s, 9H). |
| 351 | (DMSO-d₆) δ 13.1 (br s, NH), 10.0 (br s, NH), 8.4 (s, 1H), |
| | 8.2 (s, 1H), 7.9 (d, 1H), 7.5 (d, 1H), 7.3 (t, 1H), 6.5 (s, 1H) |
| | 3.2 (m, 2H), 1.3 (s, 9H), 1.1 (t, 3H). |
| 353 | δ 8.8 (m, 1H), 7.8 (m, 1H), 7.7 (m, 1H), 7.2 (m, 1H), 6.2 |
| | (br s, 1H), 4.63 (q, 2H), 3.5 (m, 2H), 2.9 (t, 2H), 2.63 (t, |
| | 2H), 1.48 (t, 3H), 1.35 (s, 6H), 1.29 (t, 3H). |
| 354 | δ 8.64 (br s. 1H), 7.91 (s, 1H), 7.71 (d, 1H), 7.40 (t, 1H), |
| | 7.28 (d, 1H), 5.90 (m, 1H), 5.76 (m, 1H), 4.96 (m, 1H), |
| | 4.45 (m, 2H), 4.28 (m, 2H), 3.03 (q, 2H), 2.20 (m, 4H), |
| | 1.93 (m, 2H), 1.74 (m, 2H), 1.31 (t, 3H). |
| 355 | δ 8.63 (br s. 1H), 7.76 (s, 1H), 7.72 (d, 1H), 7.38 (t, 1H), |
| | 7.16 (d, 1H), 4.96 (m, 1H), 3.06-3.16 (m, 8H), 2.20 (m, |
| | 4H), 1.93 (m, 2H), 1.74 (m, 2H), 1.31 (t, 3H). |
| 356 | δ 8.66 (br s. 1H), 8.06 (s, 1H), 7.83 (d, 1H), 7.54 (d, 1H), |
| | 7.39 (t, 1H), 6.41 (br s. 1H), 4.96 (m, 1H), 3.49 (m, 2H), |
| | 3.02 (q, 2H), 2.20 (m, 4H), 1.93 (m, 2H), 1.74 (m, 2H), |
| | 1.31 (t, 3H), 1.25 (t, 3H). |
| 357 | δ 8.62 (br s 1H), 8.10 (s, 1H), 7.83 (d, 1H), 7.55 (d, 1H), |
| | 7.38 (t, 1H), 6.60 (br s 1H), 4.80 (m, 1H), 3.50 (m, 2H), |
| | 3.02 (q, 2H), 1.60 (d, 6H), 1.32 (t, 3H), 1.25 (t, 3H). |
| 359 | δ 8.64 (br s 1H), 7.92 (s, 1H), 7.71 (d, 1H), 7.41 (t, 1H), |
| | 7.28 (d, 1H), 5.91 (m, 1H), 5.76 (m, 1H), 4.79 (m, 1H), |
| | 4.45 (m, 2H), 4.28 (m, 2H), 3.03 (q, 2H), 1.60 (d, 6H), |
| | 1.31 (t, 3H). |
| 361 | δ 8.88 (br s 1H), 8.58 (d, 1H), 7.18 (m, 2H), 3.40 (m, 8H), |
| | 1.70 (s, 9H), 1.32 (t, 3H). |
| 362 | δ 8.88 (br s 1H), 8.81 (dd, 1H), 7.67 (m, 1H), 7.18 (t, 1H), |
| | 6.60 (br s 1H), 3.48 (m, 2H), 3.01 (q, 2H), 1.70 (s, 9H), |
| | 1.31 (t, 3H), 1.25 (t, 3H). |
| 363 | δ 8.80 (br s 1H), 8.72 (dd, 1H), 7.30 (m, 1H), 7.19 (dd, |
| | 1H), 5.91 (m, 1H), 5.76 (m, 1H), 4.45 (m, 2H), 4.31 (m, |
| | 2H), 3.03 (q, 2H), 1.70 (s, 9H), 1.31 (t, 3H). |
| 364 | δ 8.67 (br s 1H), 7.75 (s, 1H), 7.74 (d, 1H), 7.38 (t, 1H), |
| | 7.16 (d, 1H), 3.04 (m, 8H), 1.70 (s, 9H), 1.31 (t, 3H). |
| 366 | δ 8.67 (br s 1H), 7.90 (s, 1H), 7.74 (d, 1H), 7.40 (t, 1H), |
| | 7.28 (d, 1H), 5.91 (m, 1H), 5.76 (m, 1H), 4.45 (m, 2H), |
| | 4.28 (m, 2H), 3.04 (q, 2H), 1.69 (s, 9H), 1.31 (t, 3H). |
| 368 | δ 8.88 (s, 1H), 8.60 (d, 1H), 7.19 (m, 2H), 3.05 (m, 8H), |
| | 2.01 (q, 2H), 1.68 (s, 6H), 1.31 (t, 3H), 0.76 (t, 3H). |
| 369 | δ 8.70 (s, 1H), 7.91 (s, 1H), 7.74 (d, 1H), 7.40 (t, 1H), 7.28 |
| | (d, 1H), 5.90 (m, 1H), 5.75 (m, 1H), 4.44 (br s, 2H), 4.26 |
| | (br 5, 2H), 3.03 (q, 2H), 2.00 (q, 2H), 1.66 (s, 6H), 1.31 |
| | (t, 3H), 0.74 (t, 3H). |
| 370 | δ 8.82 (s, 1H), 8.73 (d, 1H), 7.60 (m, 1H), 7.10 (dd, 1H), |
| | 6.45 (br s, 1H), 3.40 (m, 2H), 2.94 (q, 2H), 1.93 (q, 2H), |
| | 1.60 (s, 6H), 1.23 (t, 3H), 1.17 (t, 3H), 0.67 (t, 3H). |
| 371 | δ 8.80 (s, 1H), 8.72 (d, 1H), 7.60 (m, 1H), 7.40 (dd, 1H), |
| | 5.90 (m, 1H), 5.78 (m, 1H), 4.45 (br s, 2H), 4.30 (br s, |
| | 2H), 3.03 (q, 2H), 2.01 (q, 2H), 1.68 (s, 6H), 1.31 (t, 3H), |
| | 0.76 (t, 3H). |
| 372 | δ 8.68 (s, 1H), 7.76 (m, 2H), 7.38 (t, 1H), 7.16 (d, 2H), |
| | 3.05 (m, 8H), 2.00 (q, 2H), 1.66 (s, 6H), 1.31 (t, 3H), 0.74 |
| | (t, 3H). |
| 373 | δ 9.10 (dd, 1H), 8.84 (s, 1H), 7.82 (m, 1H), 7.20 (dd, 1H), |
| | 4.39 (q, 2H), 3.05 (q, 2H), 2.02 (q, 2H), 1.67 (s, 6H), 1.41 |
| | (t, 3H), 1.32 (t, 3H), 0.78 (t, 3H). |
| 374 | δ 8.62 (s, 1H), 7.74 (s, 1H), 7.54 (d, 1H), 7.35 (t, 1H), 6.98 |
| | (d, 1H), 3.04 (q, 2H), 2.01 (q, 2H), 1.67 (s, 6H), 1.31 (t, |
| | 3H), 0.74 (t, 3H). |
| 375 | δ 8.66 (s, 1H), 8.15 (d, 1H), 8.10 (s, 1H). 7.80 (d, 1H), |
| | 7.44 (t, 1H), 4.40 (q, 2H), 3.05 (q. 2H), 2.02 (q, 2H), 1.68 |
| | (s, 6H), 1.41 (t, 3H), 1.34 (t, 3H), 0.75 (t, 3H). |
| 376 | δ 8.66 (s, 1H), 8.14 (s, 1H), 7.90 (d, 1H), 7.54 (d, 1H), |
| | 7.39 (t, 1H), 6.62 (br s, 1H), 3.48 (m, 2H), 3.00 (q, 2H), |
| | 2.04 (q, 2H), 1.66 (s, 6H), 1.31 (t, 3H), 1.24 (t, 3H), 0.74 |
| | (t, 3H). |
| 377 | δ 8.90 (s, 1H), 8.80 (d, 2H), 7.68 (m, 2H), 7.26 (t, 1H), |
| | 6.28 (s, 1H), 3.42 (q 2H), 2.60 (s, 3H), 1.63 (s, 9H), 1.26 |
| | (t, 3H). |
| 378 | δ 8.80 (s, 1H), 8.60 (d, 2H), 7.28 (m, 2H), 3.10 (2 × s, 3H |
| | each), 2.60 (s, 3H), 1.63 (s, 9H). |
| 379 | δ 8.70 (s, 1H), 8.08 (s, 1H), 7.88 (d, 1H), 7.56 (d, 1H), |
| | 7.39 (t, 1H), 6.84 (br s, 1H), 4.80 (m, 1H), 4.24 (m, 2H), |
| | 3.02 (q, 2H), 2.28 (t, 1H), 1.59 (d, 6H), 1.31 (t, 3H). |
| 380 | δ 8.70 (s, 1H), 8.08 (s, 1H), 7.90 (d, 1H), 7.56 (d, 1H), |
| | 7.40 (t, 1H), 6.74 (br s, 1H), 4.25 (dd, 1H), 3.03 (q, 2H), |
| | 2.28 (t, 1H), 1.69 (s, 9H), 1.31 (t, 3H). |
| 381 | δ 8.86 (s, 1H), 8.56 (d, 1H), 7.15 (m, 2H), 3.70 (br s, 2H), |
| | 3.42 (s, br 2H), 3.03 (q, 2H), 1.69 (s, 9H), 1.60 (m, 6H), |
| | 1.31 (t, 3H). |
| 382 | δ 8.69 (s, 1H), 8.09 (s, 1H), 7.86 (d, 1H), 7.56 (d, 1H), |
| | 7.40 (t, 1H), 6.56 (br s, 1H), 5.92 (m, 1H), 5.24 (d, 1H), |
| | 5.19 (d, 1H), 4.80 (m, 1H), 4.08 (t, 2H), 3.03 (q, 2H), 1.60 |
| | (d, 6H), 1.31 (t, 3H). |
| 383 | δ 8.90 (s, 1H), 8.85 (d, 1H), 7.70 (m, 1H), 7.20 (t, 1H), |
| | 6.62 (br s, 1H), 5.92 (m, 1H), 5.24 (d, 1H), 5.19 (d, 1H), |
| | 4.07 (t, 2H), 3.02 (q, 2H), 1.70 (s, 9H), 1.31 (t, 3H). |
| 384 | δ 8.64 (s, 1H), 7.74 (d, 1H), 7.72 (s, 1H), 7.38 (t, 1H), 7.14 |
| | (d, 1H), 3.58 (m, 1H), 3.34 (m, 1H), 3.02 (m, 5H), 1.69 (s, |
| | 9H), 1.31 (t, 3H), 1.20 (m, 3H). |
| 385 | δ 8.65 (s, 1H), 7.75 (d, 1H), 7.71 (s, 1H), 7.38 (t, 1H), 7.14 |
| | (d, 1H), 4.80 (m, 1H), 3.70 (br s, 2H), 3.40 (br s, 2H), 3.03 |
| | (q, 2H), 2.40 (m, 2H), 1.50-1.54 (m, 4H), 1.60 (d, 6H), |
| | 1.31 (t, 3H). |
| 386 | δ 8.63 (s, 1H), 7.74 (s, 1H), 7.72 (d, 1H), 7.38 (t, 1H), 7.14 |
| | (d, 1H), 4.80 (m, 1H), 3.58 (m, 1H), 3.31 (m, 1H), 3.02 |
| | (m, 5H), 1.59 (d, 6H), 1.31 (t, 3H), 1.25 (m, 3H). |
| 387 | δ 8.86 (s, 1H), 8.62 (d, 1H), 7.15 (m, 2H), 5.80 (br s, 2H), |
| | 5.23 (m, 4H), 4.12 (br s, 2H), 3.88 (br s, 2H), 3.03 (q, 2H), |
| | 1.70 (s, 9H), 1.32 (t, 3H). |
| 388 | δ 8.86 (s, 1H), 8.58 (d, 1H), 7.16 (m, 2H), 3.56 (m, 1H), |
| | 3.35 (m, 1H), 3.02 (q, 4H), 1.70 (s, 9H), 1.31 (t, 3H), 1.20 |
| | (m, 3H). |
| 389 | δ 8.82 (s, 1H), 8.78 (dd, 1H), 7.62 (m, 1H), 7.12 (dd, 1H), |
| | 6.78 (br s, 1H), 4.16 (dd, 1H), 2.93 (q, 2H), 2.19 (t, 1H), |
| | 1.62 (s, 9H), 1.23 (t, 3H). |
| 390 | δ 8.64 (s, 1H), 7.79 (d, 1H), 7.73 (s, 1H), 7.36 (t, 1H), 7.17 |
| | (d, 1H), 5.80 (m, 2H), 5.22 (m, 4H), 4.79 (m, 1H), 4.33 |
| | (br s, 2H), 3.88 (br s, 2H), 3.03 (q, 2H), 1.60 (d, 6H), 1.32 |
| | (t, 3H). |
| 391 | δ 8.62 (s, 1H), 7.78 (s, 1H), 7.74 (d, 1H), 7.42 (t, 1H), 7.18 |
| | (d, 1H), 3.12 (2 × s, 3H each), 2.60 (s, 3H), 1.68 (s, 9H). |
| 392 | δ 8.68 (s, 1H), 8.04 (s, 1H), 7.84 (d, 1H), 7.52 (d, 1H), |
| | 7.40 (t, 1H), 6.40 (s, 1H), 3.52 (q, 2H), 2.60 (s, 3H), 1.68 |
| | (s, 9H), 1.23 (t, 3H). |
| 393 | δ 8.60 (s, 1H), 7.90 (s, 1H), 7.74 (d, 1H), 7.42 (t, 1H), 7.23 |
| | (d, 1H), 5.82 (m, 2H), 4.34 (m, 4H), 2.60 (s, 3H), 1.60 (s, |
| | 9H). |
| 394 | δ 8.64 (s, 1H), 8.08 (s, 1H), 7.84 (d, 1H), 7.58 (d, 1H), |
| | 7.43 (t, 1H), 6.42 (s, 1H), 4.27 (m, 2H), 2.60 (s, 3H), 2.29 |
| | (m, 1H), 1.60 (s, 9H). |
| 395 | δ 8.66 (s, 1H), 7.65 (m, 2H), 7.32 (t, 1H), 7.04 (d, 1H), |
| | 3.57 (s, 3H), 3.20 (s, 3H), 3.02 (q, 2H), 1.68 (s, 9H), 1.31 |
| | (m, 3H). |
| 396 | δ 10.18 (s, 1H), 7.80 (d, 1H), 7.72 (s, 1H), 7.42 (t, 1H), |
| | 7.24 (d, 1H), 3.61 (s, 3H), 3.27 (s, 3H), 3.22 (q, 2H), 1.69 |
| | (s, 9H), 1.32 (m, 3H). |
| 397 | δ 8.62 (s, 1H), 8.20 (s, 1H), 8.14 (d, 1H), 7.80 (d, 1H), |
| | 7.42 (t, 1H), 4.82 (m, 1H), 4.40 (q, 2H), 2.60 (s, 3H), 1.58 |
| | (d, 6H), 1.41 (t, 3H). |
| 398 | δ 9.12 (dd, 1H), 8.78 (s, 1H), 7.82 (m, 1H), 7.22 (t, 1H), |
| | 3.92 (s, 3H), 2.60 (s, 3H), 1.68 (s, 9H). |
| 399 | δ 7.72 (m, 1H), 7.60 (dd, 1H), 7.16 (t, 1H), 6.06 (s, 1H), |
| | 5.96 (s, 1H), 4.58 (q, 2H), 3.42 (q, 2H), 2.52 (s, 3H), 1.34 |
| | (t, 3H), 1.13 (s, 9H). |
| 400 | δ 8.82 (s, 1H), 8.60 (d, 1H), 7.20 (m, 2H), 4.82 (m, 1H), |
| | 3.12 (2 × s, 3H each), 2.60 (s, 3H), 1.58 (d, 6H). |
| 401 | δ 8.80 (s, 1H), 8.72 (dd, 1H), 7.72 (m, 1H), 7.22 (t, 1H), |
| | 6.43 (s, 1H), 3.48 (q, 2H), 2.60 (s, 3H), 1.68 (s, 9H), 1.26 |
| | (t, 3H). |
| 402 | δ 8.82 (s, 1H), 8.72 (d, 1H), 7.24 (m, 2H), 5.80 (m, 2H), |
| | 4.82 (m, 1H), 4.42 (m, 4H), 2.60 (s, 3H), 1.58 (d, 6H). |
| 403 | δ 8.82 (s, 1H), 8.62 (d, 1H), 7.24 (d, 2H), 4.82 (m, 1H), |
| | 3.42 (m, 2H), 3.06 (s, 3H), 2.60 (s, 3H), 1.58 (d, 6H), 1.26 |
| | (t, 3H). |
| 404 | δ 8.60 (s, 1H), 7.82 (s, 1H), 7.74 (d, 1H), 7.42 (t, 1H), 7.20 |
| | (d, 1H), 4.82 (m, 1H), 3.12 (2 × s, 3H each), 2.60 (s, 3H), |
| | 1.58 (d, 6H). |
| 405 | δ 8.68 (s, 1H), 8.12 (s, 1H), 7.84 (d, 1H), 7.52 (d, 1H), |
| | 7.40 (t, 1H), 6.34 (s, 1H), 4.82 (m, 1H), 3.52 (q, 2H), 2.60 |
| | (s, 3H), 1.58 (d, 6H), 1.23 (t, 3H). |
| 406 | δ 8.60 (s, 1H), 7.86 (s, 1H), 7.68 (d, 1H), 7.42 (t, 1H), 7.28 |
| | (d, 1H), 5.80 (m, 2H), 4.80 (m, 1H), 4.42 (m, 4H), 2.60 (s, |
| | 3H), 1.60 (d, 6H). |
| 407 | δ 8.60 (s, 1H), 7.76 (s, 1H), 7.68 (d, 1H), 7.42 (t, 1H), 7.08 |
| | (d, 1H), 4.80 (m, 1H), 3.42 (q, 2H), 3.06 (s, 3H), 2.60 (s, |
| | 3H), 1.60 (d, 6H), 1.23 (t, 3H). |
| 408 | δ 7.82 (s, 1H), 7.62 (m, 2H), 7.40 (t, 1H), 7.00 (dd, 1H), |
| | 6.53 (s, 1H), 4.56 (q, 2H), 3.22 (q, 2H), 1.46 (t, 3H), 1.42 |
| | (t, 3H). 1.38 (s, 9H). |
| 409 | δ 8.59 (s, 1H), 7.65 (m, 2H), 7.35 (t, 1H), 7.09 (d, 1H), |
| | 4.79 (m, 1H), 3.40 (s, 3H), 3.22 (s, 3H), 3.02 (q, 2H), 1.60 |
| | (d, 6H), 1.31 (t, 3H). |
| 410 | δ 10.19 (s, 1H), 7.76 (m, 2H), 7.42 (t, 1H), 7.25 (s, 1H), |
| | 4.78 (m, 1H), 3.61 (s, 3H), 3.27 (s, 3H), 3.21 (q, 2H), 1.60 |
| | (d, 6H), 1.32 (t, 3H). |
| 411 | δ 7.62 (s, 1H), 7.27 (m, 3H), 6.80 (dd, 1H), 4.72 (s, 2H), |
| | 4.56 (q, 2H), 2.53 (m, 1H), 1.38 (t, 3H), 1.36 (s, 9H). |
| 412 | δ 7.62 (s, 1H), 7.42 (m, 1H), 7.38 (t, 1H), 7.00 (dd, 1H), |
| | 6.78 (dd, 1H), 6.43 (s, 1H), 6.06 (m, 1H), 5.40 (m, 2H), |
| | 4.56 (s, 2H), 4.52 (q, 2H), 1.42 (t, 3H), 1.41 (s, 9H). |
| 413 | δ 8.60 (d, 1H), 7.82 (s, 1H), 7.46 (m, 1H), 7.26 (t, 3H), |
| | 6.52 (s, 1H), 4.58 (q, 2H), 3.82 (q, 2H), 2.26 (s, 3H), 1.34 |
| | (t, 3H), 1.13 (s, 9H). |
| 414 | δ 8.69 (s, 1H), 8.08 (s, 1H), 7.90 (d, 1H), 7.58 (d, 1H), |
| | 7.43 (t, 1H), 6.35 (s, 1H), 5.95 (m, 1H), 5.25 (m, 2H), 4.10 |
| | (m, 2H), 3.04 (q, 2H), 1.69 (s, 9H), 1.31 (t, 2H). |
| 415 | δ 8.60 (s, 1H), 7.81 (d, 1H), 7.68 (s, 1H), 7.37 (t, 1H), 7.19 |
| | (d, 1H), 5.80 (m, 2H), 5.25 (m, 4H), 4.19 (br s, 2H), 3.90 |
| | (br s, 2H), 3.04 (q, 2H), 1.69 (s, 9H), 1.31 (t, 2H). |
| 416 | δ 8.60 (s, 1H), 7.78 (d, 1H), 7.67 (s, 1H), 7.39 (t, 1H), 7.14 |
| | (d, 1H), 3.78 (m, 2H), 3.39 (m, 2H), 3.04 (q, 2H), 1.69 (s, |
| | 9H), 1.60 (m, 2H), 1.32 (t, 2H). |
| 417 | δ 8.69 (s, 1H), 8.03 (s, 1H), 7.89 (d, 1H), 7.50 (d, 1H), |
| | 7.39 (t, 1H), 6.45 (s, 1H), 3.04 (q, 2H), 2.90 (m, 1H), 1.69 |
| | (s, 9H), 1.31 (t, 3H), 0.88 (m, 2H), 0.65 (m, 2H). |
| 418 | δ 8.60 (s, 1H), 7.78 (d, 1H), 7.64 (s, 1H), 7.38 (t, 1H), 7.13 |
| | (d, 1H), 3.42 (q, 4H), 3.24 (q, 2H), 2.60 (s, 3H), 1.60 (s, |
| | 9H), 1.22 (t, 3H). |
| 419 | δ 8.60 (s, 1H), 7.66 (d, 2H), 7.60 (s, 1H), 7.35 (t, 1H), 7.07 |
| | (d, 1H), 3.59 (s, 3H), 3.22 (s, 3H), 2.60 (s, 3H), 1.68 (s, |
| | 9H). |
| 420 | δ 8.62 (s, 1H), 8.00 (s, 1H), 7.82 (m, 1H), 7.42 (d, 1H), |
| | 7.33 (t, 1H), 3.84 (q, 2H), 2.60 (s, 3H), 1.68 (s, 9H), 1.26 |
| | (t, 3H). |
| 421 | δ 9.32 (s, 1H), 8.80 (s, 1H), 8.40 (d, 1H), 7.62 (d, 1H), |
| | 4.44 (q, 2H), 2.60 (s, 3H), 1.68 (s, 9H), 1.42 (t, 3H). |
| 422 | δ 9.06 (s, 1H), 9.00 (s, 1H), 8.80 (s, 1H), 8.72 (s, 1H), 4.46 |
| | (q, 2H), 2.60 (s, 3H), 1.63 (s, 9H), 1.45 (t, 3H). |
| 423 | δ 9.32 (s, 1H), 8.39 (m, 2H), 7.08 (d, 1H), 3.15 (s, 3H), |
| | 3.00 (s, 3H), 2.60 (s, 3H), 1.68 (s, 9H). |
| 424 | δ 9.32 (s, 1H), 8.60 (s, 1H), 8.42 (d, 1H), 7.62 (d, 1H), |
| | 6.45 (s, 1H), 3.54 (q, 2H), 2.60 (s, 3H), 1.68 (s, 9H), 1.26 |
| | (t, 3H). |
| 425 | δ 9.24 (s, 1H), 8.38 (d, 1H), 8.34 (s, 1H), 7.02 (d, 1H), |
| | 3.54 (q, 2H), 3.24 (q, 2H), 2.60 (s, 3H), 1.68 (s, 9H), 1.22 |
| | (t, 3H). |
| 426 | δ 8.76 (s, 1H), 8.70 (s, 1H), 8.48 (s, 1H), 8.40 (s, 1H), 3.13 |
| | (s, 3H), 3.05 (s, 3H), 2.60 (s, 3H), 1.63 (s, 9H). |
| 427 | δ 9.06 (s, 1H), 8.90 (s, 1H), 8.88 (s, 1H), 8.60 (s, 1H), 7.02 |
| | (s, 1H), 3.48 (q, 2H), 2.60 (s, 3H), 1.63 (s, 9H), 1.15 (t, |
| | 3H). |
| 428 | δ 8.80 (s, 1H), 8.68 (s, 1H), 8.40 (s, 2H), 3.48 (q, 4H), |
| | 2.60 (s, 3H), 1.63 (s, 9H), 1.15 (t, 6H). |
| 429 | δ 8.54 (s, 1H), 7.78 (s, 1H), 7.74 (d, 1H), 7.40 (t, 1H), 7.20 |
| | (d, 1H), 3.11 (s, 3H), 3.03 (s, 3H), 1.72 (s, 9H). |
| 430 | δ 8.80 (d, 1H), 7.82 (s, 1H), 7.66 (m, 2H), 7.26 (t, 3H), |
| | 6.36 (s, 1H), 5.90 (m, 1H), 5.26 (m, 2H), 4.52 (q, 2H), |
| | 4.02 (m, 2H), 1.42 (t, 3H), 1.13 (s, 9H). |
| 431 | δ 8.42 (d, 1H), 7.82 (d, 1H), 7.26 (m, 2H), 6.52 (s, 1H), |
| | 5.90 (m, 2H), 5.26 (m, 4H), 4.52 (q, 2H), 4.02 (m, 4H), |
| | 1.42 (t, 3H), 1.13 (s, 9H). |
| 432 | δ 8.57 (s, 1H), 8.15 (d, 1H), 8.11 (s, 1H), 7.83 (d, 1H), |
| | 7.45 (t, 1H), 4.40 (q, 1H), 1.71 (s, 9H), 1.41 (t, 3H). |
| 433 | δ 8.65 (s, 1H), 7.94 (s, 1H), 7.72 (d, 1H), 7.40 (t, 1H), 7.30 |
| | (d, 1H), 5.90 (m, 1H), 5.76 (s, 1H), 4.44 (s, 2H), 4.28 (s, |
| | 2H), 1.71 (s, 9H), 1.24 (t, 3H). |
| 434 | δ 8.69 (s, 1H), 8.09 (s, 1H), 7.85 (d, 1H), 7.56 (d, 1H), |
| | 7.37 (t, 1H), 6.62 (s, 1H), 3.47 (m, 2H), 1.71 (s, 9H), 1.24 |
| | (t, 3H). |
| 435 | δ 8.67 (s, 1H), 8.06 (s, 1H), 7.84 (d, 1H), 7.54 (d, 1H), |
| | 7.36 (t, 1H), 6.73 (s, 1H), 2.88 (m, 1H), 1.71 (s, 9H), 0.82 |
| | (m, 2H), 0.65 (m, 2H). |
| 436 | δ 9.18 (br s, 1H), 8.41 (dd, 1H), 7.76 (t, 1H), 7.29 (dd, |
| | 1H) 3.15 (s, 3H), 3.04 (s, 3H), 2.60 (s, 3H), 1.68 (s, 9H). |
| 437 | δ 9.08 (br s, 1H), 8.41 (d, 1H), 7.95 (d, 1H), 7.82 (t, 1H), |
| | 7.78 (t, 1H), 3.54 (q, 2H), 2.61 (s, 3H), 1.72 (s, 9H), 1.30 |
| | (t, 3H). |
| 438 | δ 9.12 (br s, 1H), 8.46 (d, 1H), 7.97 (m, 2H), 5.92 (m, 1H), |
| | 5.40 (m, 2H), 4.13 (t, 2H), 2.60 (s, 3H), 1.72 (s, 9H). |
| 439 | δ 9.18 (br s, 1H), 8.40 (d, 1H), 7.80 (t, 1H), 7.36 (d, 1H), |
| | 5.82 (m, 2H), 5.40 (m, 4H), 4.17 (d, 2H), 3.96 (d, 2H), |
| | 2.60 (s, 3H), 1.68 (s, 9H). |
| 440 | δ 9.18 (br s, 1H), 8.40 (d, 1H), 7.85 (t, 1H), 7.22 (d, 1H), |
| | 3.58 (q, 2H), 3.31 (q, 3H), 2.60 (s, 3H), 1.70 (s, 9H), 1.30 |
| | (t, 6H). |
| 441 | δ 9.18 (br s, 1H), 8.40 (d, 1H), 7.80 (t, 1H), 7.46 (d, 1H), |
| | 5.80 (m, 2H), 4.52 (m, 4H), 2.60 (s, 3H), 1.70 (s, 9H). |
| 442 | δ 8.64 (d, 1H), 7.82 (s, 1H), 7.38 (m, 1H), 7.20 (t, 1H), |
| | 6.49 (s, 1H), 5.90 (m, 1H), 5.26 (m, 2H), 4.50 (q, 2H), |
| | 4.42 (d, 2H), 2.36 (s, 3H), 1.42 (t, 3H), 1.34 (s, 9H). |
| 443 | δ 7.74 (m, 1H), 7.62 (dd, 1H), 7.20 (t, 1H), 6.10 (s, 1H), |
| | 5.92 (s, 1H), 5.90 (m, 1H), 5.26 (m, 2H), 4.52 (q, 2H), |
| | 4.02 (m, 2H), 2.44 (s, 3H), 1.42 (t, 3H), 1.13 (s, 9H). |
| 444 | δ 9.18 (br s, 1H), 8.37 (dd, 1H), 7.80 (m, 1H), 7.29 (dd, |
| | 1H), 5.82 (m, 1H), 5.24 (m, 2H), 3.95 (d, 2H), 3.06 (s, |
| | 3H), 2.60 (s, 3H), 1.68 (s, 9H). |
| 445 | δ 8.66 (s, 1H), 7.76 (m, 2H), 7.35 (m, 2H), 7.18 (d, 1H), |
| | 6.24 (dd, 1H), 5.49 (dd, 1H), 3.11 (s, 3H), 3.02 (s, 3H), |
| | 1.71 (s, 9H). |
| 447 | δ 9.50 (br s, 1H), 8.73 (br d, 1H), 7.65 (m, 1H), 7.20 (m, |
| | 1H), 6.15 (br s, 1H), 4.68 (q, 2H), 3.50 (m, 2H), 1.51 (t, |
| | 3H), 1.39 (s, 9H), 1.27 (t, 3H). |
| 448 | δ 9.25 (br s, 1H), 7.78 (s, 1H), 7.70 (d, 1H), 7.42 (t, 1H), |
| | 7.21 (d, 1H), 6.20 (br s, 1H), 4.68 (q, 2H), 3.12 (br s, 3H), |
| | 3.01 (br s, 3H), 1.51 (t, 3H), 1.38 (s, 9H). |
| 449 | δ 9.23 (br s, 1H), 7.74 (s, 1H), 7.55 (d, 1H), 7.40 (t, 1H), |
| | 7.02 (d, 1H), 4.68 (q, 2H), 1.50 (t, 3H), 1.38 (s, 9H). |
| 450 | δ 9.45 (br s, 1H), 8.50 (d, 1H), 7.21 (m, 2H), 4.68 (q, 2H), |
| | 3.12 (br s, 3H), 3.01 (br s, 3H), 1.52 (t, 3H), 1.39 (s, 9H). |
| 451 | δ 9.45 (br s, 1H), 9.01 (dd, 1H), 7.85 (m, 1H), 7.20 (t, 1H), |
| | 4.69 (q, 2H), 4.40 (q, 2H), 1.51 (t, 3H), 1.46 (t, 3H), 1.39 |
| | (s, 9H). |
| 452 | δ 1.2 (t, 3H) 1.3 (t, 3H) 1.6 (s, 9H) 2.9 (q, 2H) 3.5 (m, 2H) |
| | 6.4 (br s, NH) 7.5 (d, 1H) 7.9 (s, 1H) 8.2 (br s, NH) 8.3 (d, |
| | 1H), 8.5 (s, 1H). |

^{a 1}H NMR data are in ppm downfield from tetramethylsilane. Couplings are designated by (s)-smglet, (d)-doublet, (t)-triplet, (q)-quartet, (m)-multiplet, (dd)-doublet of doublets, (dt)-doublet of triplets, (dq)-doublet of quartets, (br s)-broad smglet, (br d)-broad d, (br m)-broad multiplet

### Biological Examples of the Invention

Test A

Seeds of plant species selected from barnyardgrass (Echinochloa crus-galli (L.) Beauv.), downy bromegrass (Bromus tectorum L.), large crabgrass (Digitaria sanguinalis (L.) Scop.), giant foxtail (Setaria faberi Herrm.), morningglory (Ipomoea spp.), redroot pigweed (Amaranthus retroflexus L.) and velvetleaf (Abutilon theophrasti Medik.) were planted into a sandy loam soil and treated preemergence with a directed soil spray using test chemicals formulated in a non-phytotoxic solvent mixture which included a surfactant. At the same time plants selected from these species were also treated postemergence by spraying to runoff with test chemicals formulated.

Plants ranged in height from 2 to 10 cm and were in the one- to two-leaf stage for the postemergence treatment. Treated plants and untreated controls were maintained in a greenhouse for approximately ten days, after which time all treated plants were compared to untreated controls and visually evaluated for injury. Plant response ratings, summarized in Table A, are based on a 0 to 100 scale where 0 is no effect and 100 is complete control. A dash (-) response means no test results.

TABLE A
Postemergence

| | 2000 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 2 | 3 | 4 | 5 | 6 | 8 | 27 | 28 | 30 | 33 | 34 | 35 | 36 | 40 | 41 | 43 | 44 | 45 | 46 | 47 | 52 | 53 | 54 | 55 | 58 | 59 | 60 |
| Barnyardgrass | 100 | 100 | 100 | 90 | 90 | 0 | 75 | 65 | 70 | 90 | 80 | 85 | 80 | 0 | 60 | 100 | 100 | 100 | 100 | 100 | 55 | 0 | 10 | 100 | 80 | 30 | 70 |
| Crabgrass, Large | 100 | 95 | 70 | 50 | 90 | 0 | 70 | 20 | 25 | 85 | 50 | 60 | 10 | 0 | 60 | 90 | 80 | 90 | 90 | 100 | 20 | 0 | 15 | 100 | 75 | 20 | 55 |
| Foxtail, Giant | 100 | 100 | 80 | 45 | 70 | 0 | 70 | 25 | 55 | 85 | 40 | 70 | 35 | 0 | 35 | 70 | 90 | 95 | 100 | 85 | 25 | 0 | 5 | 80 | 60 | 20 | 60 |
| Morningglory | 80 | 20 | 35 | 15 | 30 | 0 | 100 | 25 | 0 | 25 | 20 | 10 | 10 | 0 | 10 | 30 | 45 | 25 | 65 | 25 | 0 | 0 | 10 | 25 | 0 | 10 | 20 |
| Pigweed | 100 | 100 | 100 | 90 | 100 | 45 | 90 | 80 | 65 | 85 | 90 | 95 | 85 | 0 | 60 | 100 | 100 | 100 | 100 | 100 | 35 | 0 | 20 | 100 | 90 | 75 | 95 |
| Velvetleaf | 75 | 60 | 45 | 20 | 95 | 5 | 45 | 25 | 20 | 40 | 30 | 40 | 20 | 0 | 25 | 30 | 65 | 65 | 75 | 40 | 20 | 0 | 5 | 40 | 40 | 20 | 20 |
| | 2000 g ai/ha | | | | | | | | | | | | | | | | | | | | | | | | | | |
| | Compounds | | | | | | | | | | | | | | | | | | | | | | | | | | |
| | 61 | 62 | 63 | 64 | 65 | 66 | 67 | 70 | 71 | 78 | 79 | 80 | 82 | 83 | 84 | 85 | 86 | 87 | 88 | 89 | 90 | 91 | 92 | 93 | 94 | 95 | 96 |
| Barnyardgrass | 90 | 100 | 90 | 75 | 75 | 0 | 25 | 30 | 0 | 40 | 40 | 90 | 20 | 20 | 10 | 25 | 20 | 55 | 100 | 20 | 20 | 0 | 0 | 25 | 35 | 55 | 80 |
| Crabgrass, Large | 45 | 60 | 80 | 60 | 60 | 0 | 20 | 30 | 0 | 35 | 55 | 90 | 30 | 25 | 20 | 30 | 25 | 60 | 95 | 25 | 25 | 20 | 10 | 15 | 30 | 60 | 70 |
| Foxtail, Giant | 85 | 85 | 80 | 55 | 55 | 0 | 20 | 20 | 0 | 60 | 40 | 90 | 20 | 20 | 10 | 30 | 20 | 50 | 60 | 20 | 20 | 0 | 0 | 10 | 15 | 50 | 80 |
| Morningglory | 20 | 20 | 15 | 10 | 10 | 0 | 20 | 30 | 0 | 25 | 25 | 50 | 10 | 10 | 10 | 25 | 35 | 20 | 40 | 15 | 10 | 0 | 0 | 10 | 20 | 15 | 15 |
| Pigweed | 95 | 100 | 100 | 95 | 70 | 10 | 85 | 75 | 0 | 90 | 85 | 100 | 50 | 55 | 45 | 70 | 95 | 35 | 100 | 30 | 40 | 20 | 0 | 30 | 30 | 100 | 100 |
| Velvetleaf | 15 | 20 | 55 | 30 | 25 | 0 | 15 | 30 | 0 | 50 | 50 | 75 | 30 | 20 | 20 | 20 | 30 | 25 | 55 | 20 | 30 | 10 | 0 | 15 | 25 | 50 | 70 |
| | 2000 g ai/ha | | | | | | | | | | | | | | | | | | | | | | | | | | |
| | Compounds | | | | | | | | | | | | | | | | | | | | | | | | | | |
| | 97 | 98 | 102 | 103 | 107 | 108 | 109 | 111 | 112 | 114 | 115 | 116 | 117 | 120 | 121 | 122 | 123 | 124 | 125 | 127 | 128 | 130 | 136 | 137 | 138 | 139 | 143 |
| Barnyardgrass | 10 | 0 | 75 | 75 | 55 | 100 | 90 | 0 | 45 | 40 | 80 | 25 | 75 | 65 | 35 | 85 | 20 | 0 | 0 | 50 | 80 | 20 | 60 | 25 | 30 | 60 | 25 |
| Crabgrass, Large | 10 | 0 | 80 | 30 | 40 | 85 | 55 | 10 | 30 | 60 | 80 | 10 | 65 | 65 | 10 | 65 | 10 | 10 | 0 | 35 | 70 | 20 | 70 | 60 | 30 | 30 | 30 |
| Foxtail, Giant | 10 | 0 | 65 | 30 | 35 | 100 | 90 | 0 | 35 | 55 | 80 | 0 | 55 | 20 | 25 | 80 | 10 | 0 | 0 | 30 | 60 | 10 | 50 | 30 | 35 | 40 | 15 |
| Morningglory | 0 | 0 | 55 | 35 | 40 | 40 | 40 | 0 | 15 | 25 | 30 | 0 | 70 | 40 | 20 | 65 | 10 | 0 | 0 | 20 | 15 | 25 | 40 | 40 | 40 | 35 | 25 |
| Pigweed | 25 | 10 | 100 | 100 | 100 | 100 | 100 | 10 | 60 | 100 | 100 | 0 | 95 | 85 | 100 | 100 | 15 | 40 | 20 | 50 | 80 | 65 | 85 | 100 | 100 | 100 | 35 |
| Velvetleaf | 10 | 5 | 20 | 45 | 0 | 100 | 45 | 0 | 35 | 20 | 40 | 0 | 65 | 30 | 15 | 70 | 10 | 10 | 0 | 0 | 35 | 20 | 75 | 70 | 60 | 40 | 15 |
| | 2000 g ai/ha | | | | | | | | | | | | | | | | | | | | | | | | | | |
| | Compounds | | | | | | | | | | | | | | | | | | | | | | | | | | |
| | 144 | 145 | 146 | 147 | 148 | 149 | 150 | 151 | 153 | 154 | 164 | 165 | 166 | 167 | 172 | 179 | 180 | 189 | 190 | 195 | 196 | 197 | 198 | 199 | 200 | 201 | 202 |
| Barnyardgrass | 100 | 100 | 75 | 95 | 25 | 65 | 65 | 10 | 100 | 90 | 10 | 0 | 10 | 0 | 0 | 10 | 30 | 10 | 0 | 50 | 30 | 0 | 80 | 80 | 35 | 20 | 80 |
| Crabgrass, Large | 90 | 85 | 55 | 90 | 10 | 75 | 50 | 10 | 20 | 30 | 10 | 0 | 0 | 5 | 0 | 10 | 35 | 10 | 15 | 25 | 0 | 10 | 65 | 50 | 25 | 20 | 65 |
| Foxtail, Giant | 100 | 60 | 40 | 90 | 15 | 65 | 25 | 10 | 90 | 20 | 0 | 0 | 20 | 0 | 0 | 10 | 20 | 0 | 0 | 0 | 0 | 5 | 65 | 65 | 20 | 10 | 50 |
| Morningglory | 35 | 30 | 0 | 30 | 10 | 60 | 10 | 20 | 85 | 40 | 5 | 5 | 10 | 10 | 10 | 30 | 15 | 10 | 25 | 10 | 0 | 0 | 75 | 70 | 45 | 30 | 75 |
| Pigweed | 100 | 100 | 100 | 100 | 15 | 90 | 75 | 70 | 100 | 100 | 0 | 5 | 25 | 0 | 60 | 10 | 95 | 45 | 20 | 25 | 0 | 5 | 75 | 80 | 60 | 55 | 90 |
| Velvetleaf | 75 | 55 | 35 | 50 | 15 | 40 | 15 | 60 | 65 | 30 | 0 | 20 | 30 | 20 | 15 | 5 | 15 | 5 | 15 | 15 | 0 | 5 | 35 | 35 | 10 | 10 | 30 |
| | 2000 g ai/ha | | | | | | | | | | | | | | | | | | | | | | | | | | |
| | Compounds | | | | | | | | | | | | | | | | | | | | | | | | | | |
| | 203 | 204 | 205 | 206 | 207 | 209 | 210 | 211 | 212 | 213 | 214 | 215 | 216 | 217 | 222 | 223 | 255 | 267 | 268 | 269 | 270 | 271 | 272 | 273 | 277 | 278 | 279 |
| Barnyardgrass | 30 | 30 | 65 | 10 | 70 | 70 | 80 | 55 | 90 | 45 | 15 | 25 | 15 | 0 | 90 | 55 | 70 | 65 | 80 | 80 | 30 | 30 | 90 | 75 | 50 | 60 | 40 |
| Crabgrass, Large | 15 | 30 | 20 | 5 | 65 | 40 | 65 | 30 | 60 | 30 | 10 | 30 | 20 | 20 | 85 | 25 | 40 | 75 | 75 | 80 | 10 | 10 | 90 | 65 | 0 | 5 | 20 |
| Foxtail, Giant | 20 | 25 | 25 | 0 | 50 | 25 | 75 | 30 | 80 | 25 | 40 | 40 | 20 | 0 | 85 | 10 | 55 | 55 | 80 | 65 | 0 | 20 | 95 | 70 | 0 | 15 | 20 |
| Morningglory | 55 | 15 | 20 | 0 | 15 | 15 | 25 | 15 | 20 | 10 | 10 | 35 | 20 | 25 | 95 | 60 | 25 | 45 | 40 | 35 | 20 | 30 | 100 | 75 | 0 | 5 | 5 |
| Pigweed | 80 | 60 | 65 | 20 | 45 | 50 | 80 | 25 | 90 | 40 | 50 | 80 | 55 | 25 | 100 | 55 | 100 | 60 | 85 | 50 | 0 | 90 | 100 | 100 | 35 | 70 | 60 |
| Velvetleaf | 10 | 60 | 30 | 20 | 25 | 20 | 65 | 25 | 35 | 25 | 20 | 30 | 35 | 30 | 100 | 35 | 50 | 35 | 45 | 30 | 25 | 40 | 100 | 95 | 30 | 25 | 20 |
| | 2000 g ai/ha | | | | | | | | | | | | | | | | | | | | | | | | | | |
| | Compounds | | | | | | | | | | | | | | | | | | | | | | | | | | |
| | 280 | 281 | 282 | 283 | 284 | 285 | 286 | 287 | 288 | 289 | 290 | 291 | 300 | 312 | 313 | 314 | 315 | 316 | 317 | 318 | 319 | 329 | 338 | 347 | 348 | 354 | 355 |
| Barnyardgrass | 25 | 65 | 80 | 35 | 60 | 80 | 80 | 90 | 65 | 40 | 70 | 40 | 90 | 0 | 95 | 75 | 20 | 45 | 0 | 90 | 60 | 20 | 35 | 0 | 0 | 70 | 75 |
| Crabgrass, Large | 0 | 10 | 25 | 10 | 20 | 35 | 25 | 60 | 15 | 20 | 10 | 25 | 90 | 0 | 100 | 60 | 20 | 45 | 10 | 70 | 50 | 5 | 45 | 0 | 0 | 20 | 30 |
| Foxtail, Giant | 0 | 25 | 25 | 25 | 35 | 55 | 75 | 90 | 10 | 70 | 70 | 75 | 90 | 0 | 80 | 70 | 0 | 30 | 0 | 90 | 40 | 20 | 30 | 0 | 0 | 50 | 45 |
| Morningglory | 10 | 15 | 15 | 0 | 15 | 50 | 35 | 15 | 20 | 20 | 30 | 10 | 95 | 0 | 55 | 50 | 0 | 30 | 0 | 55 | 25 | 20 | 70 | 0 | 0 | 85 | 75 |
| Pigweed | 70 | 70 | 100 | 100 | 100 | 100 | 85 | 80 | 40 | 50 | 60 | 30 | 100 | 0 | 100 | 100 | 20 | 80 | 10 | 100 | 40 | 45 | 95 | 0 | 0 | 80 | 90 |
| Velvetleaf | 30 | 40 | 50 | 35 | 35 | 75 | 50 | 65 | 20 | 5 | 30 | 10 | 80 | 0 | 50 | 30 | 0 | 20 | 0 | 50 | 30 | 25 | 60 | 0 | 0 | 70 | 65 |
| | 2000 g ai/ha | | | 1000 g ai/ha | | | | | | | | 500 g ai/ha | | | | | | | | | | | | | | | |
| | Compounds | | | Compounds | | | | | | | | Compounds | | | | | | | | | | | | | | | |
| | 356 | 413 | 417 | 31 | 32 | 39 | 131 | 132 | 133 | 134 | 135 | 2 | 3 | 4 | 5 | 6 | 8 | 27 | 28 | 30 | 33 | 34 | 35 | 36 | 38 | 40 | 41 |
| Barnyardgrass | 40 | 50 | 80 | 75 | 0 | 55 | 60 | 30 | 55 | 70 | 50 | 100 | 100 | 80 | 30 | 80 | 0 | 80 | 65 | 70 | 75 | 45 | 70 | 35 | 55 | 0 | 30 |
| Crabgrass, Large | 15 | 15 | 85 | 40 | 0 | 35 | 40 | 25 | 20 | 35 | 30 | 85 | 75 | 20 | 20 | 80 | 0 | 50 | 30 | 25 | 65 | 30 | 55 | 10 | 60 | 15 | 20 |
| Foxtail, Giant | 10 | 35 | 85 | 70 | 0 | 45 | 15 | 20 | 20 | 35 | 30 | 95 | 95 | 20 | 25 | 70 | 0 | 60 | 20 | 20 | 70 | 30 | 50 | 20 | 60 | 0 | 20 |
| Morningglory | 20 | 20 | 85 | 20 | 0 | 0 | 15 | 20 | 20 | 20 | 10 | 45 | 25 | 15 | 10 | 10 | 0 | 35 | 5 | 0 | 10 | 10 | 10 | 10 | 0 | 0 | 10 |
| Pigweed | 70 | 85 | 100 | 80 | 0 | 75 | 75 | 65 | 75 | 100 | 85 | 100 | 100 | 95 | 25 | 100 | 20 | 90 | 70 | 60 | 85 | 90 | 85 | 80 | 70 | 0 | 40 |
| Velvetleaf | 10 | 55 | 90 | 30 | 0 | 10 | 10 | 30 | 15 | 20 | 25 | 40 | 40 | 20 | 10 | 90 | 5 | 25 | 25 | 25 | 30 | 20 | 25 | 15 | 20 | 0 | 25 |
| | 500 g ai/ha | | | | | | | | | | | | | | | | | | | | | | | | | | |
| | Compounds | | | | | | | | | | | | | | | | | | | | | | | | | | |
| | 43 | 44 | 45 | 46 | 47 | 51 | 52 | 53 | 54 | 55 | 58 | 59 | 60 | 61 | 62 | 63 | 64 | 65 | 66 | 67 | 70 | 71 | 78 | 79 | 80 | 82 | 83 |
| Barnyardgrass | 90 | 90 | 100 | 100 | 90 | 75 | 25 | 0 | 10 | 90 | 70 | 20 | 35 | 35 | 65 | 60 | 30 | 45 | 0 | 0 | 25 | 0 | 30 | 40 | 90 | 20 | 5 |
| Crabgrass, Large | 65 | 55 | 80 | 75 | 85 | 30 | 0 | 5 | 10 | 25 | 40 | 15 | 20 | 30 | 25 | 40 | 30 | 30 | 0 | 20 | 25 | 0 | 20 | 20 | 70 | 20 | 30 |
| Foxtail, Giant | 70 | 50 | 90 | 80 | 90 | 40 | 25 | 0 | 10 | 30 | 45 | 15 | 30 | 20 | 55 | 50 | 30 | 30 | 0 | 10 | 20 | 0 | 20 | 30 | 80 | 20 | 20 |
| Morningglory | 20 | 15 | 25 | 40 | 20 | 35 | 0 | 0 | 0 | 20 | 10 | 10 | 10 | 10 | 10 | 15 | 10 | 10 | 0 | 5 | 25 | 0 | 15 | 15 | 40 | 20 | 10 |
| Pigweed | 100 | 100 | 100 | 100 | 100 | 85 | 30 | 0 | 20 | 90 | 70 | 40 | 40 | 85 | 100 | 95 | 65 | 70 | 10 | 50 | 60 | 0 | 85 | 95 | 100 | 50 | 45 |
| Velvetleaf | 15 | 45 | 40 | 45 | 25 | 40 | 10 | 0 | 0 | 35 | 20 | 15 | 20 | 15 | 10 | 40 | 20 | 25 | 0 | 20 | 20 | 0 | 45 | 45 | 60 | 20 | 20 |
| | 500 g ai/ha | | | | | | | | | | | | | | | | | | | | | | | | | | |
| | Compounds | | | | | | | | | | | | | | | | | | | | | | | | | | |
| | 84 | 85 | 86 | 87 | 88 | 89 | 90 | 91 | 92 | 93 | 94 | 95 | 96 | 97 | 98 | 102 | 103 | 107 | 108 | 109 | 110 | 111 | 112 | 114 | 115 | 116 | 117 |
| Barnyardgrass | 15 | 20 | 20 | 15 | 85 | 20 | 20 | 0 | 0 | 20 | 20 | 30 | 60 | 5 | 0 | 55 | 45 | 20 | 85 | 75 | 30 | 0 | 35 | 30 | 70 | 0 | 45 |
| Crabgrass, Large | 20 | 20 | 20 | 20 | 85 | 20 | 15 | 10 | 0 | 10 | 10 | 25 | 30 | 10 | 0 | 60 | 15 | 25 | 65 | 20 | 30 | 10 | 25 | 40 | 75 | 0 | 40 |
| Foxtail, Giant | 15 | 20 | 20 | 20 | 20 | 20 | 20 | 0 | 0 | 10 | 10 | 20 | 60 | 5 | 0 | 45 | 10 | 20 | 90 | 55 | 25 | 0 | 20 | 50 | 60 | 0 | 35 |
| Morningglory | 5 | 20 | 20 | 15 | 35 | 20 | 10 | 0 | 0 | 5 | 20 | 10 | 10 | 0 | 0 | 35 | 25 | 30 | 40 | 25 | 10 | 5 | 20 | 10 | 35 | 0 | 20 |
| Pigweed | 50 | 75 | 55 | 10 | 90 | 20 | 20 | 0 | 0 | 20 | 25 | 85 | 95 | 0 | 5 | 95 | 85 | 95 | 100 | 100 | 50 | 5 | 40 | 85 | 95 | 0 | 80 |
| Velvetleaf | 20 | 30 | 40 | 10 | 40 | 20 | 10 | 5 | 0 | 10 | 20 | 45 | 70 | 5 | 0 | 10 | 10 | 0 | 90 | 25 | 10 | 0 | 25 | 20 | 40 | 0 | 50 |
| | 500 g ai/ha | | | | | | | | | | | | | | | | | | | | | | | | | | |
| | Compounds | | | | | | | | | | | | | | | | | | | | | | | | | | |
| | 120 | 121 | 122 | 123 | 124 | 125 | 127 | 128 | 130 | 136 | 137 | 138 | 139 | 143 | 144 | 145 | 146 | 147 | 148 | 149 | 150 | 151 | 153 | 154 | 164 | 165 | 166 |
| Barnyardgrass | 45 | 10 | 75 | 5 | 0 | 0 | 30 | 70 | 20 | 55 | 20 | 15 | 35 | 10 | 85 | 40 | 50 | 55 | 20 | 45 | 30 | 10 | 75 | 50 | 10 | 0 | 0 |
| Crabgrass, Large | 50 | 10 | 35 | 10 | 10 | 0 | 15 | 60 | 20 | 35 | 30 | 20 | 0 | 20 | 55 | 30 | 15 | 40 | 5 | 40 | 15 | 5 | 10 | 20 | 10 | 0 | 0 |
| Foxtail, Giant | 20 | 15 | 80 | 0 | 0 | 0 | 30 | 45 | 10 | 25 | 20 | 20 | 20 | 10 | 80 | 40 | 15 | 45 | 15 | 40 | 20 | 10 | 20 | 20 | 0 | 0 | 0 |
| Morningglory | 20 | 20 | 30 | 0 | 5 | 0 | 10 | 10 | 20 | 20 | 25 | 15 | 20 | 10 | 25 | 15 | 5 | 20 | 15 | 30 | 0 | 10 | 35 | 20 | 10 | 5 | 0 |
| Pigweed | 50 | 90 | 60 | 0 | 30 | 20 | 40 | 50 | 50 | 65 | 90 | 60 | 75 | 20 | 100 | 90 | 85 | 90 | 0 | 65 | 80 | 35 | 95 | 90 | 0 | 0 | 20 |
| Velvetleaf | 25 | 10 | 45 | 0 | 10 | 0 | 0 | 20 | 20 | 55 | 60 | 20 | 25 | 0 | 55 | 30 | 20 | 30 | 20 | 30 | 25 | 35 | 40 | 10 | 5 | 10 | 20 |
| | 500 g ai/ha | | | | | | | | | | | | | | | | | | | | | | | | | | |
| | Compounds | | | | | | | | | | | | | | | | | | | | | | | | | | |
| | 167 | 172 | 179 | 180 | 189 | 190 | 195 | 196 | 197 | 198 | 199 | 200 | 201 | 202 | 203 | 204 | 205 | 206 | 207 | 209 | 210 | 211 | 212 | 213 | 214 | 215 | 216 |
| Barnyardgrass | 0 | 0 | 5 | 20 | 0 | 0 | 15 | 0 | 10 | 45 | 10 | 10 | 10 | 45 | 10 | 25 | 45 | 5 | 40 | 30 | 80 | 25 | 50 | 25 | 0 | 0 | 0 |
| Crabgrass, Large | 0 | 0 | 10 | 20 | 10 | 10 | 10 | 0 | 5 | 20 | 15 | 10 | 10 | 30 | 10 | 30 | 10 | 0 | 20 | 20 | 40 | 20 | 45 | 20 | 5 | 20 | 20 |
| Foxtail, Giant | 0 | 0 | 0 | 15 | 0 | 0 | 5 | 0 | 0 | 30 | 25 | 0 | 0 | 30 | 10 | 25 | 10 | 0 | 10 | 15 | 40 | 20 | 45 | 20 | 15 | 25 | 20 |
| Morningglory | 0 | 10 | 10 | 10 | 10 | 10 | 10 | 5 | 0 | 60 | 45 | 35 | 25 | 75 | 50 | 20 | 25 | 0 | 20 | 0 | 20 | 20 | 15 | 20 | 0 | 25 | 20 |
| Pigweed | 0 | 25 | 0 | 30 | 30 | 10 | 5 | 0 | 0 | 55 | 80 | 50 | 40 | 50 | 35 | 50 | 50 | 15 | 30 | 40 | 60 | 25 | 75 | 30 | 20 | 70 | 30 |
| Velvetleaf | 10 | 5 | 0 | 10 | 5 | 0 | 0 | 0 | 5 | 30 | 30 | 10 | 10 | 30 | 10 | 40 | 20 | 5 | 20 | 10 | 40 | 15 | 35 | 30 | 15 | 30 | 25 |
| | 500 g ai/ha | | | | | | | | | | | | | | | | | | | | | | | | | | |
| | Compounds | | | | | | | | | | | | | | | | | | | | | | | | | | |
| | 217 | 222 | 223 | 255 | 267 | 268 | 269 | 270 | 271 | 272 | 273 | 277 | 278 | 279 | 280 | 281 | 282 | 283 | 284 | 285 | 286 | 287 | 288 | 289 | 290 | 291 | 300 |
| Barnyardgrass | 0 | 75 | 10 | 40 | 40 | 80 | 70 | 0 | 10 | 55 | 55 | 0 | 30 | 20 | 0 | 30 | 40 | 10 | 30 | 55 | 60 | 70 | 5 | 10 | 55 | 25 | 75 |
| Crabgrass, Large | 0 | 80 | 20 | 20 | 45 | 50 | 35 | 0 | 10 | 55 | 25 | 0 | 10 | 10 | 5 | 5 | 10 | 10 | 20 | 15 | 20 | 10 | 0 | 20 | 10 | 5 | 55 |
| Foxtail, Giant | 0 | 65 | 10 | 30 | 30 | 55 | 35 | 0 | 10 | 45 | 50 | 0 | 5 | 10 | 0 | 0 | 20 | 10 | 20 | 25 | 35 | 40 | 0 | 10 | 15 | 30 | 55 |
| Morningglory | 15 | 80 | 50 | 10 | 30 | 20 | 20 | 20 | 10 | 100 | 70 | 0 | 10 | 5 | 0 | 10 | 15 | 0 | 10 | 40 | 20 | 20 | 20 | 10 | 20 | 5 | 80 |
| Pigweed | 10 | 75 | 50 | 70 | 40 | 35 | 35 | 20 | 45 | 100 | 100 | 10 | 60 | 70 | 55 | 70 | 75 | 80 | 95 | 80 | 55 | 50 | 20 | 20 | 30 | 20 | 90 |
| Velvetleaf | 5 | 80 | 40 | 35 | 30 | 45 | 25 | 15 | 10 | 100 | 90 | 25 | 20 | 20 | 20 | 35 | 50 | 15 | 30 | 60 | 30 | 55 | 20 | 0 | 20 | 5 | 60 |
| | 500 g ai/ha | | | | | | | | | | | | | | | | | | | | | | | | | | |
| | Compounds | | | | | | | | | | | | | | | | | | | | | | | | | | |
| | 311 | 312 | 313 | 314 | 315 | 316 | 317 | 318 | 319 | 329 | 338 | 347 | 348 | 354 | 355 | 356 | 413 | 417 | 446 | 447 | 448 | 449 | 450 | 451 | 452 | 453 | 454 |
| Barnyardgrass | 10 | 0 | 80 | 35 | 20 | 20 | 0 | 65 | 10 | 15 | 20 | 0 | 0 | 20 | 40 | 25 | 20 | 65 | 90 | 70 | 90 | 35 | 80 | 20 | 35 | 15 | 80 |
| Crabgrass, Large | 10 | 0 | 70 | 20 | 20 | 25 | 10 | 30 | 10 | 10 | 20 | 0 | 0 | 10 | 10 | 10 | 10 | 30 | 65 | 25 | 80 | 30 | 70 | 30 | 20 | 10 | 70 |
| Foxtail, Giant | 0 | 0 | 70 | 30 | 0 | 20 | 0 | 50 | 0 | 10 | 20 | 0 | 0 | 10 | 10 | 10 | 10 | 75 | 90 | 40 | 90 | 50 | 75 | 20 | 25 | 10 | 70 |
| Morningglory | 0 | 0 | 30 | 30 | 0 | 20 | 0 | 50 | 15 | 10 | 25 | 0 | 0 | 20 | 15 | 10 | 10 | 70 | 40 | 10 | 55 | 15 | 30 | 20 | 30 | 10 | 50 |
| Pigweed | 0 | 0 | 100 | 80 | 10 | 25 | 0 | 95 | 40 | 10 | 70 | 0 | 0 | 70 | 40 | 65 | 60 | 100 | 80 | 50 | 90 | 40 | 55 | 50 | 70 | 20 | 70 |
| Velvetleaf | 10 | 0 | 40 | 20 | 0 | 15 | 0 | 40 | 20 | 10 | 40 | 0 | 0 | 50 | 15 | 10 | 40 | 60 | 60 | 30 | 80 | 20 | 60 | 30 | 30 | 10 | 70 |

| | 250 g ai/ha | | | | | | | | 125 g ai/ha | | | | | | | | | | | | |
|---|---|---|---|---|---|---|---|---|---|---|---|---|---|---|---|---|---|---|---|---|---|
| | Compounds | | | | | | | | Compounds | | | | | | | | | | | | |
| | 31 | 32 | 39 | 131 | 132 | 133 | 134 | 135 | 38 | 51 | 110 | 311 | 446 | 447 | 448 | 449 | 450 | 451 | 452 | 453 | 454 |
| Barnyardgrass | 65 | 0 | 10 | 25 | 20 | 20 | 30 | 30 | 40 | 50 | 30 | 0 | 80 | 25 | 90 | 20 | 60 | 10 | 10 | 10 | 40 |
| Crabgrass, Large | 30 | 0 | 10 | 20 | 20 | 10 | 20 | 20 | 30 | 30 | 25 | 0 | 45 | 10 | 75 | 20 | 15 | 10 | 10 | 10 | 10 |
| Foxtail, Giant | 45 | 0 | 10 | 10 | 15 | 10 | 15 | 20 | 50 | 20 | 20 | 0 | 75 | 10 | 85 | 20 | 50 | 10 | 10 | 10 | 20 |
| Morningglory | 5 | 0 | 0 | 15 | 10 | 10 | 10 | 10 | 0 | 10 | 0 | 0 | 10 | 5 | 15 | 5 | 25 | 20 | 20 | 10 | 10 |
| Pigweed | 55 | 0 | 70 | 45 | 75 | 30 | 85 | 80 | 50 | 50 | 40 | 0 | 65 | 40 | 70 | 20 | 20 | 10 | 20 | 10 | 40 |
| Velvetleaf | 20 | 0 | 10 | 15 | 20 | 10 | 20 | 15 | 10 | 40 | 10 | 0 | 30 | 10 | 60 | 10 | 30 | 20 | 20 | 5 | 50 |

Preemergence

| | 2000 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 2 | 3 | 4 | 5 | 6 | 8 | 27 | 28 | 30 | 33 | 34 | 35 | 36 | 40 | 41 | 43 | 44 | 45 | 46 | 47 | 52 | 53 | 54 | 55 | 58 | 59 | 60 |
| Barnyardgrass | 100 | 90 | 95 | 95 | 100 | 10 | 90 | 65 | 75 | 80 | 80 | 90 | 75 | 15 | 75 | 90 | 90 | 95 | 100 | 95 | 90 | 10 | 25 | 95 | 75 | 60 | 85 |
| Crabgrass, Large | 100 | 90 | 95 | 95 | 100 | 10 | 90 | 55 | 60 | 95 | 100 | 100 | 70 | 15 | 80 | 90 | 90 | 75 | 90 | 90 | 90 | 40 | 35 | 100 | 95 | 45 | 95 |
| Foxtail, Giant | 100 | 100 | 95 | 80 | 100 | 5 | 100 | 45 | 65 | 95 | 85 | 100 | 85 | 5 | 70 | 100 | 100 | 100 | 100 | 100 | 60 | 0 | 15 | 100 | 95 | 65 | 90 |
| Morningglory | 100 | 90 | 60 | 20 | 80 | — | 80 | 30 | 50 | 70 | 50 | 85 | 70 | 10 | 45 | 40 | 30 | 75 | 65 | 30 | 10 | 0 | 15 | 20 | 40 | 40 | 40 |
| Pigweed | 100 | 100 | 100 | 100 | 100 | 75 | 100 | 90 | 100 | 100 | 100 | 100 | 100 | 15 | 85 | 100 | 100 | 100 | 100 | 100 | 90 | 40 | 65 | 100 | 100 | 100 | 100 |
| Velvetleaf | 100 | 90 | 100 | 75 | 100 | 25 | 100 | 75 | 55 | 80 | 25 | 95 | 90 | 15 | 40 | 60 | 35 | 65 | 85 | 95 | 50 | 0 | 0 | 70 | 65 | 55 | 95 |
| | 2000 g ai/ha | | | | | | | | | | | | | | | | | | | | | | | | | | |
| | Compounds | | | | | | | | | | | | | | | | | | | | | | | | | | |
| | 61 | 62 | 63 | 64 | 65 | 66 | 67 | 70 | 71 | 78 | 79 | 80 | 82 | 83 | 84 | 85 | 86 | 87 | 88 | 89 | 90 | 91 | 92 | 93 | 94 | 95 | 96 |
| Barnyardgrass | 75 | 95 | 100 | 85 | 70 | 20 | 30 | 20 | 0 | 45 | 35 | 35 | 0 | 0 | 0 | 0 | 0 | 90 | 90 | 0 | 50 | 15 | 0 | 40 | 65 | 50 | 90 |
| Crabgrass, Large | 90 | 85 | 100 | 100 | 100 | 0 | 55 | 45 | 0 | 90 | 55 | 30 | 0 | 0 | 0 | 5 | 0 | 95 | 100 | 0 | 75 | 35 | 0 | 70 | 60 | 90 | 100 |
| Foxtail, Giant | 75 | 100 | 100 | 90 | 80 | 0 | 30 | 55 | 0 | 70 | 40 | 70 | 0 | 0 | 0 | 0 | 0 | 90 | 90 | 0 | 60 | 30 | 0 | 60 | 65 | 90 | 95 |
| Morningglory | 50 | 75 | 70 | 5 | 5 | 0 | 30 | 10 | 0 | 40 | 60 | 30 | 15 | 10 | — | 5 | 15 | 15 | 40 | 10 | 30 | 10 | 0 | 20 | 10 | 55 | 70 |
| Pigweed | 65 | 100 | 100 | 100 | 100 | 0 | 90 | 5 | 0 | 90 | 90 | 90 | 35 | 0 | 0 | 5 | 55 | 95 | 100 | 10 | 70 | 55 | 35 | 70 | 85 | 100 | 100 |
| Velvetleaf | 85 | 95 | 70 | 35 | 30 | 0 | 75 | 20 | 0 | 50 | 70 | 10 | 0 | 0 | 0 | 0 | 10 | 65 | 70 | 10 | 35 | 20 | 0 | 25 | 25 | 80 | 100 |
| | 2000 g ai/ha | | | | | | | | | | | | | | | | | | | | | | | | | | |
| | Compounds | | | | | | | | | | | | | | | | | | | | | | | | | | |
| | 97 | 98 | 102 | 103 | 107 | 108 | 109 | 111 | 112 | 114 | 115 | 116 | 117 | 120 | 121 | 122 | 123 | 124 | 125 | 127 | 128 | 130 | 136 | 137 | 138 | 139 | 143 |
| Barnyardgrass | 0 | 0 | 85 | 85 | 35 | 90 | 90 | 10 | 50 | 40 | 85 | 70 | 70 | 85 | 40 | 95 | 50 | 10 | 0 | 30 | 85 | 30 | 85 | 60 | 80 | 60 | 0 |
| Bromegrass, Downy | | | 60 | — | — | — | — | — | — | — | — | — | — | — | — | — | | | | | | | | | | | |
| Crabgrass, Large | 0 | 0 | 100 | 55 | 90 | 100 | 95 | 30 | 55 | 85 | 100 | 80 | 75 | 75 | 75 | 95 | 0 | 15 | 0 | 85 | 100 | 60 | 90 | 90 | 90 | 75 | 15 |
| Foxtail, Giant | 0 | 0 | 85 | 15 | 80 | 100 | 90 | 75 | 50 | 45 | 80 | 35 | 60 | 55 | 75 | 85 | 0 | 0 | 0 | 35 | 65 | 55 | 70 | 80 | 90 | 75 | 0 |
| Morningglory | 0 | 0 | 90 | 10 | 100 | 75 | 45 | 0 | 45 | 70 | 90 | 10 | 75 | 55 | 20 | 30 | 0 | 15 | 0 | 30 | 85 | 15 | 45 | 35 | 45 | 85 | 0 |
| Pigweed | 0 | 0 | 90 | 95 | 95 | 100 | 100 | 10 | 90 | 90 | 100 | 0 | 90 | 50 | 100 | 90 | 0 | 75 | 0 | 50 | 95 | 65 | 80 | 90 | 95 | 100 | 0 |
| Velvetleaf | 0 | 0 | 60 | 20 | 0 | 90 | 25 | 0 | 50 | 60 | 95 | 0 | 65 | 40 | 30 | 70 | 0 | 15 | 0 | 30 | 85 | 30 | 30 | 35 | 50 | 80 | 0 |
| | 2000 g ai/ha | | | | | | | | | | | | | | | | | | | | | | | | | | |
| | Compounds | | | | | | | | | | | | | | | | | | | | | | | | | | |
| | 144 | 145 | 146 | 147 | 148 | 149 | 150 | 151 | 153 | 154 | 164 | 165 | 166 | 167 | 172 | 179 | 180 | 189 | 190 | 195 | 196 | 197 | 198 | 199 | 200 | 201 | 202 |
| Barnyardgrass | 100 | 100 | 80 | 95 | 20 | 90 | 80 | 0 | 90 | 95 | 0 | 0 | 10 | 0 | 0 | 40 | 0 | 65 | 40 | 45 | 55 | 0 | 90 | 95 | 45 | 50 | 80 |
| Crabgrass, Large | 90 | 90 | 80 | 90 | 15 | 100 | 50 | 0 | 25 | 85 | 0 | 0 | 30 | 5 | 0 | 50 | 50 | 75 | 70 | 95 | 50 | 0 | 90 | 100 | 75 | 70 | 100 |
| Foxtail, Giant | 100 | 100 | 45 | 95 | 10 | 100 | 55 | 0 | 60 | 70 | 0 | 0 | 10 | 10 | 0 | 10 | 10 | 20 | 10 | 45 | 20 | 0 | 95 | 100 | 60 | 15 | 80 |
| Morningglory | 75 | 25 | 50 | 45 | 5 | 65 | 35 | 0 | 55 | 30 | 0 | 0 | 5 | 5 | 0 | 5 | 10 | 30 | 25 | 10 | 10 | 5 | 85 | 55 | 60 | 5 | 70 |
| Pigweed | 100 | 100 | 95 | 100 | 25 | 95 | 100 | 0 | 100 | 100 | 0 | 20 | 10 | 50 | 40 | 35 | 90 | 40 | 85 | 0 | 15 | 0 | 100 | 100 | 95 | 95 | 100 |
| Velvetleaf | 90 | 90 | 25 | 85 | 10 | 25 | 50 | 10 | 80 | 60 | 0 | 5 | 5 | 10 | 0 | 0 | 15 | 25 | 15 | 15 | 0 | 10 | 85 | 90 | 35 | 35 | 85 |
| | 2000 g ai/ha | | | | | | | | | | | | | | | | | | | | | | | | | | |
| | Compounds | | | | | | | | | | | | | | | | | | | | | | | | | | |
| | 203 | 204 | 205 | 206 | 207 | 209 | 210 | 211 | 212 | 213 | 214 | 215 | 216 | 217 | 222 | 223 | 255 | 267 | 268 | 269 | 270 | 271 | 272 | 273 | 277 | 278 | 279 |
| Barnyardgrass | 60 | 5 | 90 | 60 | 80 | 80 | 90 | 70 | 85 | 70 | 30 | 35 | 35 | 0 | 100 | 80 | 75 | 80 | 100 | 100 | 90 | 75 | 100 | 95 | 70 | 80 | 55 |
| Crabgrass, Large | 75 | 10 | 95 | 20 | 90 | 95 | 100 | 85 | 95 | 95 | 65 | 55 | 55 | 10 | 95 | 90 | 55 | 90 | 100 | 100 | 35 | 15 | 100 | 100 | 25 | 60 | 70 |
| Foxtail, Giant | 75 | 10 | 100 | 20 | 90 | 90 | 100 | 70 | 95 | 85 | 55 | 75 | 70 | 10 | 100 | 90 | 60 | 85 | 100 | 100 | 70 | 70 | 100 | 100 | 55 | 85 | 60 |
| Morningglory | 15 | 25 | 45 | 25 | 85 | 40 | 100 | 25 | 65 | 60 | 30 | 55 | 15 | 0 | 100 | 55 | 70 | 45 | 25 | 30 | 30 | 0 | 55 | 80 | 0 | 25 | 20 |
| Pigweed | 90 | 10 | 100 | 60 | 100 | 100 | 100 | 40 | 100 | 95 | 95 | 85 | 70 | 40 | 90 | 90 | 100 | 80 | 100 | 100 | 85 | 60 | 100 | 100 | 70 | 95 | 100 |
| Velvetleaf | 35 | 25 | 100 | 10 | 100 | 75 | 95 | 65 | 100 | 35 | 20 | 30 | 30 | 5 | 100 | 85 | 50 | 60 | 100 | 100 | 55 | 5 | 60 | 75 | 45 | 35 | 30 |
| | 2000 g ai/ha | | | | | | | | | | | | | | | | | | | | | | | | | | |
| | Compounds | | | | | | | | | | | | | | | | | | | | | | | | | | |
| | 280 | 281 | 282 | 283 | 284 | 285 | 286 | 287 | 288 | 289 | 290 | 291 | 300 | 312 | 313 | 314 | 315 | 316 | 317 | 318 | 319 | 329 | 338 | 347 | 348 | 354 | 355 |
| Barnyardgrass | 45 | 75 | 75 | 30 | 85 | 90 | 85 | 100 | 75 | 50 | 90 | 25 | 100 | 0 | 90 | 90 | 5 | 10 | 0 | 95 | 80 | 5 | 95 | 0 | 0 | 55 | 80 |
| Crabgrass, Large | 25 | 65 | 65 | 90 | 80 | 85 | 70 | 100 | 10 | 55 | 25 | 10 | 100 | 0 | 95 | 90 | 15 | 35 | 10 | 70 | 100 | 0 | 100 | 0 | 0 | 20 | 50 |
| Foxtail, Giant | 45 | 75 | 40 | 50 | 55 | 90 | 100 | 100 | 75 | 40 | 90 | 0 | 100 | 0 | 90 | 90 | 10 | 0 | 0 | 85 | 80 | 5 | 100 | 0 | 0 | 80 | 60 |
| Morningglory | 15 | 25 | 40 | 10 | 5 | 45 | 40 | 50 | 30 | 15 | 40 | 0 | 100 | 0 | 55 | 30 | 10 | 0 | 0 | 50 | 60 | 10 | 40 | 0 | 0 | 20 | 40 |
| Pigweed | 90 | 95 | 90 | 90 | 100 | 100 | 95 | 100 | 70 | 75 | 95 | 35 | 100 | 0 | 100 | 90 | 45 | 65 | 30 | 100 | 100 | 10 | 90 | 0 | 0 | 70 | 50 |
| Velvetleaf | 30 | 35 | 45 | 15 | 40 | 40 | 70 | 100 | 40 | 20 | 30 | 0 | 100 | 0 | 80 | 80 | 30 | 15 | 30 | 80 | 45 | 10 | 50 | 0 | 0 | 45 | 55 |
| | 2000 g ai/ha | | | 1000 g ai/ha | | | | | | | | 500 g ai/ha | | | | | | | | | | | | | | | |
| | Compounds | | | Compounds | | | | | | | | Compounds | | | | | | | | | | | | | | | |
| | 356 | 413 | 417 | 31 | 32 | 39 | 131 | 132 | 133 | 134 | 135 | 2 | 3 | 4 | 5 | 6 | 8 | 27 | 28 | 30 | 33 | 34 | 35 | 36 | 38 | 40 | 41 |
| Barnyardgrass | 45 | 75 | 85 | 80 | 0 | 25 | 50 | 10 | 45 | 45 | 65 | 100 | 90 | 90 | 50 | 100 | 0 | 75 | 45 | 45 | 55 | 55 | 70 | 45 | 5 | 0 | 40 |
| Crabgrass, Large | 20 | 70 | 95 | 45 | 0 | 85 | 85 | 35 | 80 | 95 | 90 | 100 | 95 | 90 | 80 | 100 | 10 | 85 | 75 | 50 | 90 | 60 | 95 | 10 | 10 | 5 | 40 |
| Foxtail, Giant | 45 | 90 | 100 | 50 | 0 | 40 | 75 | 40 | 30 | 45 | 50 | 100 | 100 | 90 | 40 | 100 | 10 | 90 | 30 | 30 | 80 | 40 | 80 | 5 | 0 | 0 | 30 |
| Morningglory | 15 | 15 | 80 | 55 | 0 | 20 | 35 | 10 | 0 | 30 | 25 | 80 | 55 | 15 | 10 | 45 | 0 | 55 | 45 | 25 | 35 | 35 | 25 | 20 | 10 | 5 | 20 |
| Pigweed | 80 | 60 | 100 | 80 | 0 | 100 | 90 | 40 | 65 | 100 | 85 | 100 | 100 | 100 | 80 | 100 | 40 | 100 | 95 | 90 | 100 | 100 | 100 | 95 | 0 | 0 | 65 |
| Velvetleaf | 20 | 35 | 70 | 35 | 0 | 20 | 60 | 20 | 35 | 40 | 50 | 100 | 95 | 80 | 25 | 100 | 0 | 75 | 60 | 50 | 20 | 20 | 35 | 20 | 5 | 5 | 25 |
| | 500 g ai/ha | | | | | | | | | | | | | | | | | | | | | | | | | | |
| | Compounds | | | | | | | | | | | | | | | | | | | | | | | | | | |
| | 43 | 44 | 45 | 46 | 47 | 51 | 52 | 53 | 54 | 55 | 58 | 59 | 60 | 61 | 62 | 63 | 64 | 65 | 66 | 67 | 70 | 71 | 78 | 79 | 80 | 82 | 83 |
| Barnyardgrass | 60 | 75 | 80 | 90 | 90 | 85 | 25 | 0 | 5 | 80 | 65 | 25 | 50 | 40 | 50 | 70 | 55 | 55 | 0 | 0 | 20 | 0 | 20 | 20 | 10 | 0 | 0 |
| Crabgrass, Large | 75 | 45 | 55 | 65 | 55 | 80 | 70 | 0 | 25 | 75 | 70 | 25 | 75 | 75 | 70 | 90 | 70 | 95 | 0 | 15 | 35 | 0 | 60 | 50 | 10 | 0 | 0 |
| Foxtail, Giant | 55 | 65 | 75 | 90 | 90 | 100 | 20 | 0 | 0 | 60 | 85 | 5 | 55 | 20 | 75 | 95 | 40 | 40 | 0 | 0 | 40 | 0 | 55 | 10 | 20 | 0 | 0 |
| Morningglory | 10 | 10 | 15 | 40 | 20 | 15 | 0 | 0 | 0 | 5 | 20 | 10 | 10 | 20 | 20 | 10 | 0 | 5 | 0 | 0 | 20 | 0 | 25 | 20 | 15 | 0 | 30 |
| Pigweed | 100 | 100 | 95 | 100 | 100 | 100 | 90 | 30 | 10 | 100 | 100 | 90 | 95 | 95 | 100 | 100 | 100 | 100 | 0 | 0 | 0 | 0 | 80 | 45 | 70 | 5 | 0 |
| Velvetleaf | 5 | 20 | 25 | 30 | 20 | 100 | 15 | 0 | 0 | 20 | 70 | 15 | 65 | 55 | 70 | 20 | 10 | 10 | 0 | 30 | 10 | 0 | 30 | 20 | 10 | 0 | 0 |
| | 500 g ai/ha | | | | | | | | | | | | | | | | | | | | | | | | | | |
| | Compounds | | | | | | | | | | | | | | | | | | | | | | | | | | |
| | 84 | 85 | 86 | 87 | 88 | 89 | 90 | 91 | 92 | 93 | 94 | 95 | 96 | 97 | 98 | 102 | 103 | 107 | 108 | 109 | 110 | 111 | 112 | 114 | 115 | 116 | 117 |
| Barnyardgrass | 0 | 0 | 0 | 55 | 75 | 0 | 20 | 0 | 0 | 25 | 20 | 30 | 70 | 0 | 0 | 70 | 55 | 0 | 85 | 85 | 5 | 0 | 25 | 25 | 70 | 35 | 30 |
| Bromegrass, Downy | | | | | | | | | | | | | | — | — | 60 | — | — | — | — | — | — | — | — | — | — | — |
| Crabgrass, Large | 0 | 0 | 0 | 90 | 45 | 0 | 35 | 0 | 0 | 35 | 35 | 50 | 80 | 0 | 0 | 95 | 35 | 65 | 85 | 80 | 20 | 25 | 45 | 40 | 100 | 45 | 45 |
| Foxtail, Giant | 0 | 0 | 0 | 35 | 55 | 0 | 25 | 0 | 0 | 35 | 45 | 70 | 75 | 0 | 0 | 60 | 0 | 10 | 95 | 80 | 0 | 45 | 25 | 30 | 50 | 20 | 25 |
| Morningglory | 0 | 0 | 0 | 15 | 10 | 0 | 0 | 0 | 0 | 5 | 10 | 30 | 25 | 0 | 0 | 65 | 0 | 95 | 30 | 35 | 20 | 0 | 20 | 35 | 70 | 0 | 45 |
| Pigweed | 0 | 0 | 0 | 60 | 90 | 0 | 25 | 0 | 0 | 40 | 35 | 100 | 100 | 0 | 0 | 80 | 80 | 35 | 100 | 75 | 10 | 10 | 65 | 85 | 95 | 0 | 55 |
| Velvetleaf | 0 | 0 | 0 | 25 | 45 | 0 | 20 | 5 | 0 | 10 | 5 | 50 | 95 | 0 | 0 | 30 | 20 | 0 | 55 | 15 | 10 | 0 | 25 | 35 | 85 | 0 | 50 |
| | 500 g ai/ha | | | | | | | | | | | | | | | | | | | | | | | | | | |
| | Compounds | | | | | | | | | | | | | | | | | | | | | | | | | | |
| | 120 | 121 | 122 | 123 | 124 | 125 | 127 | 128 | 130 | 136 | 137 | 138 | 139 | 143 | 144 | 145 | 146 | 147 | 148 | 149 | 150 | 151 | 153 | 154 | 164 | 165 | 166 |
| Barnyardgrass | 30 | 15 | 90 | 5 | 0 | 0 | 20 | 55 | 40 | 65 | 30 | 30 | 40 | 0 | 100 | 95 | 70 | 40 | 0 | 70 | 50 | 0 | 80 | 30 | 0 | 0 | 0 |
| Crabgrass, Large | 45 | 10 | 70 | 15 | 30 | 0 | 75 | 90 | 45 | 85 | 55 | 70 | 45 | 0 | 90 | 70 | 70 | 85 | 0 | 95 | 25 | 0 | 25 | 25 | 0 | 0 | 0 |
| Foxtail, Giant | 25 | 25 | 65 | 0 | 0 | 0 | 20 | 15 | 50 | 60 | 50 | 30 | 60 | 0 | 90 | 85 | 45 | 60 | 0 | 70 | 15 | 0 | 15 | 20 | 0 | 0 | 0 |
| Morningglory | 20 | 5 | 25 | 0 | 5 | 0 | 30 | 45 | 10 | 20 | 20 | 15 | 30 | 0 | 20 | 20 | 5 | 20 | 0 | 30 | 10 | 0 | 10 | 10 | 0 | 0 | 0 |
| Pigweed | 20 | 95 | 85 | 0 | 20 | 0 | 40 | 60 | 35 | 70 | 60 | 80 | 100 | 0 | 100 | 95 | 85 | 85 | 0 | 90 | 75 | 0 | 100 | 85 | 0 | 0 | 0 |
| Velvetleaf | 20 | 10 | 40 | 0 | 0 | 0 | 10 | 75 | 20 | 25 | 20 | 20 | 50 | 0 | 70 | 75 | 20 | 25 | 0 | 0 | 20 | 0 | 15 | 15 | 0 | 0 | 0 |
| | 500 g ai/ha | | | | | | | | | | | | | | | | | | | | | | | | | | |
| | Compounds | | | | | | | | | | | | | | | | | | | | | | | | | | |
| | 167 | 172 | 179 | 180 | 189 | 190 | 195 | 196 | 197 | 198 | 199 | 200 | 201 | 202 | 203 | 204 | 205 | 206 | 207 | 209 | 210 | 211 | 212 | 213 | 214 | 215 | 216 |
| Barnyardgrass | 0 | 0 | 0 | 0 | 35 | 20 | 20 | 15 | 0 | 55 | 55 | 35 | 0 | 45 | 10 | 0 | 80 | 10 | 70 | 60 | 85 | 55 | 80 | 40 | 0 | 15 | 15 |
| Crabgrass, Large | 0 | 0 | 0 | 10 | 55 | 40 | 75 | 25 | 0 | 80 | 70 | 70 | 35 | 85 | 50 | 0 | 80 | 20 | 95 | 75 | 90 | 75 | 90 | 55 | 55 | 30 | 15 |
| Foxtail, Giant | 0 | 0 | 0 | 0 | 5 | 0 | 30 | 0 | 0 | 90 | 80 | 35 | 0 | 55 | 30 | 0 | 85 | 0 | 75 | 50 | 100 | 45 | 70 | 60 | 60 | 50 | 25 |
| Morningglory | 0 | 0 | 0 | 0 | 15 | 10 | 5 | 0 | 0 | 45 | 15 | 15 | 0 | 15 | 10 | 10 | 30 | 5 | 35 | 20 | 55 | 10 | 30 | 25 | 5 | 20 | 0 |
| Pigweed | 0 | 0 | 0 | 0 | 25 | 25 | 0 | 0 | 0 | 95 | 100 | 80 | 40 | 100 | 90 | 0 | 100 | 25 | 100 | 75 | 100 | 30 | 100 | 75 | 20 | 30 | 0 |
| Velvetleaf | 5 | 0 | 0 | 0 | 10 | 0 | 0 | 0 | 0 | 45 | 35 | 20 | 0 | 35 | 15 | 5 | 70 | 15 | 30 | 10 | 70 | 25 | 65 | 15 | 5 | 15 | 10 |
| | 500 g ai/ha | | | | | | | | | | | | | | | | | | | | | | | | | | |
| | Compounds | | | | | | | | | | | | | | | | | | | | | | | | | | |
| | 217 | 222 | 223 | 255 | 267 | 268 | 269 | 270 | 271 | 272 | 273 | 277 | 278 | 279 | 280 | 281 | 282 | 283 | 284 | 285 | 286 | 287 | 288 | 289 | 290 | 291 | 300 |
| Barnyardgrass | 0 | 95 | 55 | 55 | 70 | 100 | 95 | 60 | 5 | 70 | 70 | 0 | 50 | 5 | 0 | 60 | 30 | 0 | 10 | 70 | 75 | 95 | 20 | 0 | 40 | 0 | 90 |
| Crabgrass, Large | 0 | 100 | 90 | 35 | 15 | 85 | 65 | 5 | 0 | 85 | 75 | 0 | 25 | 70 | 0 | 20 | 40 | 15 | 15 | 60 | 15 | 75 | 0 | 0 | 5 | 0 | 90 |
| Foxtail, Giant | 0 | 95 | 75 | 45 | 75 | 100 | 100 | 20 | 0 | 90 | 85 | 0 | 60 | 25 | 5 | 60 | 10 | 10 | 20 | 60 | 60 | 65 | 15 | 10 | 50 | 0 | 95 |
| Morningglory | 0 | 75 | 35 | 30 | 20 | 5 | 20 | 0 | 0 | 20 | 45 | 0 | 10 | 10 | 0 | 5 | 10 | 0 | 0 | 25 | 30 | 10 | 5 | 0 | 10 | 0 | 90 |
| Pigweed | 0 | 90 | 95 | 90 | 5 | 100 | 100 | 15 | 60 | 100 | 100 | 40 | 85 | 95 | 15 | 90 | 90 | 60 | 85 | 100 | 85 | 100 | 25 | 0 | 10 | 0 | 95 |
| Velvetleaf | 0 | 95 | 55 | 30 | 25 | 95 | 40 | 30 | 0 | 15 | 60 | 5 | 20 | 10 | 0 | 10 | 20 | 0 | 10 | 35 | 20 | 50 | 10 | 0 | 15 | 0 | 85 |
| | 500 g ai/ha | | | | | | | | | | | | | | | | | | | | | | | | | | |
| | Compounds | | | | | | | | | | | | | | | | | | | | | | | | | | |
| | 311 | 312 | 313 | 314 | 315 | 316 | 317 | 318 | 319 | 329 | 338 | 347 | 348 | 354 | 355 | 356 | 413 | 417 | 446 | 447 | 448 | 449 | 450 | 451 | 452 | 453 | 454 |
| Barnyardgrass | 0 | 0 | 80 | 50 | 5 | 0 | 0 | 65 | 55 | 0 | 60 | 0 | 0 | 0 | 50 | 5 | 60 | 60 | 85 | 60 | 100 | 90 | 95 | 55 | 70 | 45 | 85 |
| Crabgrass, Large | 0 | 0 | 85 | 55 | 5 | 10 | 0 | 55 | 85 | 0 | 65 | 0 | 0 | 10 | 20 | 10 | 40 | 75 | 40 | 35 | 85 | 80 | 70 | 25 | 45 | 15 | 85 |
| Foxtail, Giant | 0 | 0 | 75 | 60 | 0 | 0 | 0 | 40 | 55 | 0 | 80 | 0 | 0 | 15 | 20 | 20 | 65 | 90 | 95 | 45 | 100 | 90 | 95 | 45 | 45 | 0 | 85 |
| Morningglory | 0 | 0 | 20 | 10 | 5 | 0 | 0 | 20 | 20 | 0 | 10 | 0 | 0 | 15 | 10 | 5 | 5 | 20 | 15 | 0 | 60 | 5 | 15 | 5 | 10 | 0 | 20 |
| Pigweed | 0 | 0 | 95 | 80 | 15 | 0 | 20 | 75 | 100 | 5 | 55 | 0 | 0 | 60 | 10 | 25 | 45 | 95 | 100 | 50 | 100 | 50 | 95 | 0 | 65 | 10 | 90 |
| Velvetleaf | 0 | 0 | 65 | 40 | 10 | 5 | 0 | 30 | 35 | 0 | 20 | 0 | 0 | 5 | 25 | 0 | 35 | 55 | 20 | 0 | 85 | 10 | 35 | 20 | 5 | 0 | 20 |

| | 250 g ai/ha | | | | | | | | 125 g ai/ha | | | | | | | | | | | | |
|---|---|---|---|---|---|---|---|---|---|---|---|---|---|---|---|---|---|---|---|---|---|
| | Compounds | | | | | | | | Compounds | | | | | | | | | | | | |
| | 31 | 32 | 39 | 131 | 132 | 133 | 134 | 135 | 38 | 51 | 110 | 311 | 446 | 447 | 448 | 449 | 450 | 451 | 452 | 453 | 454 |
| Barnyardgrass | 40 | 0 | 15 | 45 | 0 | 0 | 25 | 35 | 0 | 60 | 10 | 0 | 55 | 20 | 95 | 45 | 60 | 15 | 15 | 20 | 70 |
| Crabgrass, Large | 20 | 0 | 35 | 20 | 20 | 25 | 35 | 40 | 0 | 80 | 15 | 0 | 10 | 0 | 45 | 35 | 35 | 5 | 20 | 10 | 50 |
| Foxtail, Giant | 20 | 0 | 20 | 10 | 25 | 0 | 20 | 10 | 0 | 70 | 0 | 0 | 70 | 10 | 80 | 60 | 70 | 5 | 5 | 0 | 10 |
| Morningglory | 0 | 0 | 0 | 15 | 5 | 0 | 5 | 0 | 5 | 10 | 10 | 0 | 10 | 0 | 60 | 0 | 0 | 0 | 0 | 0 | 0 |
| Pigweed | 45 | 0 | 55 | 40 | 30 | 10 | 35 | 55 | 0 | 100 | 10 | 0 | 90 | 10 | 100 | 0 | 25 | 0 | 10 | 10 | 20 |
| Velvetleaf | 10 | 0 | 10 | 10 | 15 | 5 | 15 | 5 | 0 | 20 | 10 | 0 | 10 | 0 | 15 | 0 | 0 | 0 | 0 | 0 | 0 |

Test B

Seeds selected from barnyardgrass (Echinochloa crus-galli (L.) Beauv.), Surinam grass (Urochloa decumbens (Staph) R. D. Webster, previously named Brachiaria decumbens Stapf), cocklebur (Xanthium strumarium L.), corn (Zea mays L.), large crabgrass (Digitaria sanguinalis (L.) Scop.), giant foxtail (Setaria faberi Herim.), lambsquarters (Chenopodium album L.), morningglory (Ipomoea coccinea L.), pigweed (Amaranthus retroflexus L.), rice (Oryza sativa L.) and velvetleaf (Abutilon theophrasti Medik.) were planted and treated preemergence with test chemicals formulated in a non-phytotoxic solvent mixture which included a surfactant.

At the same time, plants selected from these crop and weed species and also blackgrass (Alopecurus myosuroides Huds.), wheat (Triticum aestivum L.) and wild oat (Avena fatua L.) were treated with postemergence applications of test chemicals formulated in the same manner. Plants ranged in height from 2 to 18 cm (1- to 4-leaf stage) for postemergence treatments. Plant species in the flooded paddy test consisted of rice (Oryza sativa L.), small flower umbrella sedge (Cyperus difformis L.), ducksalad (Heteranthera limosa (Sw.) Willd.) and barnyardgrass (Echinochloa crus-galli (L.) Beauv.) grown to the 2-leaf stage for testing. Treated plants and controls were maintained in a greenhouse for 13 to 15 days, after which time all species were compared to controls and visually evaluated. Plant response ratings, summarized in Table B, are based on a scale of 0 to 100 where 0 is no effect and 100 is complete control. A dash (-) response means no test result.

TABLE B
Flooded Paddy

| | 1000 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 |
| Barnyardgrass | 80 | 90 | 80 | 80 | 50 | 90 | 80 | 0 | 70 | 80 | 80 | 10 | 80 | 90 | 60 | 80 | 70 | 80 | 20 | 40 | 70 | 0 | 0 | 0 | 0 | 0 | 80 |
| Ducksalad | 90 | 90 | 80 | 90 | 80 | 90 | 90 | 0 | 80 | 90 | 70 | 80 | 90 | 90 | 90 | 90 | 90 | 90 | 70 | 90 | 90 | 0 | 0 | 0 | 0 | 0 | 90 |
| Rice | 80 | 70 | 80 | 70 | 40 | 80 | 70 | 20 | 80 | 80 | 90 | 50 | 70 | 80 | 60 | 90 | 80 | 80 | 0 | 70 | 60 | 0 | 0 | 0 | 0 | 0 | 80 |
| Sedge, Umbrella | 90 | 90 | 90 | 90 | 80 | 90 | 90 | 0 | 80 | 70 | 70 | 30 | 70 | 50 | 80 | 70 | 70 | 70 | 60 | 80 | 60 | 0 | 0 | 50 | 0 | 0 | 90 |
| | 1000 g ai/ha | | | | | | | | | | | | | | | | | | | | | | | | | | |
| | Compounds | | | | | | | | | | | | | | | | | | | | | | | | | | |
| | 28 | 29 | 30 | 31 | 32 | 33 | 34 | 35 | 36 | 37 | 40 | 41 | 42 | 43 | 44 | 45 | 46 | 47 | 48 | 49 | 50 | 51 | 52 | 54 | 55 | 56 | 57 |
| Barnyardgrass | 80 | 90 | 60 | 80 | 0 | 90 | 80 | 90 | 70 | 30 | 0 | 50 | 0 | 80 | 70 | 80 | 90 | 90 | 80 | 70 | 70 | 90 | 20 | 0 | 90 | 80 | 60 |
| | 80 | 90 | 60 | 80 | 0 | 90 | 80 | 90 | 70 | 30 | 0 | 50 | 0 | 80 | 70 | 80 | 90 | 90 | 80 | 70 | 70 | 90 | 20 | 0 | 90 | 80 | 60 |
| Ducksalad | 80 | 80 | 80 | 80 | 0 | 90 | 80 | 80 | 90 | 60 | 0 | 60 | 50 | 70 | 70 | 80 | 90 | 80 | 80 | 80 | 70 | 80 | 20 | 0 | 80 | 80 | 50 |
| Rice | 80 | 70 | 60 | 80 | 0 | 60 | 50 | 60 | 40 | 50 | 0 | 20 | 0 | 50 | 70 | 70 | 70 | 70 | 80 | 30 | 60 | 70 | 0 | 0 | 70 | 70 | 60 |
| Sedge, Umbrella | 80 | 80 | 70 | 50 | 20 | 90 | 80 | 90 | 60 | 90 | 30 | 70 | 50 | 70 | 70 | 70 | 80 | 80 | 80 | 80 | 80 | 80 | 40 | 0 | 80 | 80 | 60 |
| | 1000 g ai/ha | | | | | | | | | | | | | | | | | | | | | | | | | | |
| | Compounds | | | | | | | | | | | | | | | | | | | | | | | | | | |
| | 58 | 59 | 60 | 61 | 62 | 63 | 64 | 65 | 66 | 67 | 68 | 69 | 70 | 71 | 72 | 73 | 74 | 75 | 76 | 77 | 78 | 79 | 80 | 81 | 82 | 83 | 84 |
| Barnyardgrass | 80 | 0 | 60 | 30 | 70 | 80 | 30 | 80 | 10 | 60 | 0 | 0 | 10 | 0 | 0 | 30 | 0 | 80 | 70 | 40 | 30 | 60 | 80 | 50 | 30 | 10 | 10 |
| Ducksalad | 80 | 0 | 70 | 80 | 80 | 90 | 90 | 90 | 0 | 80 | 0 | 0 | 0 | 0 | 0 | 10 | 0 | 90 | 100 | 40 | 30 | 80 | 90 | 80 | 20 | 30 | 20 |
| Rice | 60 | 0 | 50 | 0 | 30 | 60 | 50 | 50 | 0 | 60 | 0 | 0 | 10 | 0 | 0 | 50 | 0 | 90 | 60 | 70 | 40 | 70 | 60 | 60 | 40 | 10 | 0 |
| Sedge, Umbrella | 80 | 0 | 60 | 60 | 80 | 100 | 90 | 90 | 10 | 80 | 0 | 0 | 30 | 0 | 0 | 80 | 70 | 70 | 60 | 80 | 80 | 90 | 80 | 80 | 40 | 50 | 70 |
| | 1000 g ai/ha | | | | | | | | | | | | | | | | | | | | | | | | | | |
| | Compounds | | | | | | | | | | | | | | | | | | | | | | | | | | |
| | 85 | 86 | 87 | 88 | 89 | 90 | 91 | 92 | 93 | 94 | 95 | 96 | 97 | 98 | 99 | 100 | 101 | 102 | 104 | 105 | 106 | 107 | 108 | 109 | 110 | 112 | 113 |
| Barnyardgrass | 40 | 30 | 20 | 70 | 10 | 10 | 0 | 0 | 0 | 20 | 50 | 80 | 0 | 0 | 0 | 0 | 70 | 80 | 0 | 20 | 0 | 0 | 80 | 80 | 30 | 50 | 70 |
| Ducksalad | 20 | 20 | 20 | 80 | 0 | 30 | 30 | 0 | 10 | 30 | 80 | 90 | 50 | 0 | 0 | 0 | 90 | 90 | 60 | 70 | 80 | 60 | 90 | 90 | 0 | 80 | 90 |
| Rice | 40 | 50 | 10 | 40 | 10 | 10 | 10 | 0 | 20 | 10 | 60 | 70 | 0 | 0 | 0 | 0 | 80 | 50 | 0 | 0 | 0 | 0 | 70 | 70 | 20 | 70 | 60 |
| Sedge, Umbrella | 60 | 70 | 80 | 80 | 60 | 50 | 60 | 0 | 70 | 50 | 80 | 90 | 70 | 0 | 40 | 0 | 80 | 100 | 70 | 70 | 90 | 60 | 90 | 60 | 20 | 80 | 90 |
| | 1000 g ai/ha | | | | | | | | | | | | | | | | | | | | | | | | | | |
| | Compounds | | | | | | | | | | | | | | | | | | | | | | | | | | |
| | 114 | 115 | 117 | 118 | 119 | 120 | 121 | 122 | 123 | 124 | 125 | 126 | 127 | 128 | 129 | 130 | 131 | 132 | 133 | 134 | 135 | 136 | 137 | 138 | 139 | 140 | 141 |
| Barnyardgrass | 70 | 90 | 70 | 50 | 0 | 30 | 0 | 60 | 0 | 0 | 0 | 10 | 50 | 70 | 30 | 40 | 70 | 30 | 20 | 60 | 60 | 60 | 20 | 10 | 70 | 70 | 10 |
| Ducksalad | 80 | 90 | 80 | 80 | 0 | 70 | 0 | 80 | 50 | 70 | 0 | 80 | 90 | 90 | 70 | 10 | 80 | 10 | 50 | 90 | 80 | 90 | 30 | 80 | 80 | 80 | 80 |
| Rice | 50 | 60 | 50 | 80 | 0 | 0 | 0 | 70 | 0 | 0 | 0 | 20 | 50 | 60 | 10 | 0 | 60 | 0 | 0 | 60 | 60 | 30 | 20 | 50 | 50 | 30 | 0 |
| Sedge, Umbrella | 90 | 90 | 90 | 80 | 0 | 10 | 0 | 80 | 30 | 0 | 0 | 80 | 100 | 90 | 80 | 0 | 80 | 40 | 40 | 80 | 70 | 90 | 90 | 80 | 80 | 80 | 90 |
| | 1000 g ai/ha | | | | | | | | | | | | | | | | | | | | | | | | | | |
| | Compounds | | | | | | | | | | | | | | | | | | | | | | | | | | |
| | 142 | 143 | 144 | 145 | 146 | 147 | 148 | 149 | 150 | 151 | 152 | 153 | 154 | 155 | 156 | 157 | 158 | 159 | 160 | 161 | 162 | 163 | 164 | 165 | 166 | 167 | 168 |
| Barnyardgrass | 90 | 0 | 80 | 70 | 60 | 60 | 20 | 70 | 70 | 0 | 80 | 40 | 50 | 70 | 100 | 20 | 70 | 80 | 70 | 80 | 80 | 70 | 0 | 0 | 0 | 0 | 50 |
| Ducksalad | 90 | 0 | 90 | 70 | 80 | 80 | 0 | 90 | 20 | 0 | 80 | 80 | 80 | 100 | 100 | 0 | 90 | 90 | 90 | 80 | 80 | 80 | 0 | 0 | 0 | 0 | 70 |
| Rice | 70 | 0 | 70 | 40 | 40 | 50 | 0 | 50 | 40 | 0 | 70 | 60 | 20 | 20 | 90 | 30 | 60 | 60 | 90 | 40 | 80 | 60 | 0 | 0 | 0 | 0 | 40 |
| Sedge, Umbrella | 80 | 0 | 90 | 90 | 90 | 90 | 70 | 60 | 50 | 60 | 90 | 70 | 20 | 90 | 90 | 40 | 80 | 70 | 80 | 60 | 80 | 70 | 0 | 0 | 30 | 0 | 90 |
| | 1000 g ai/ha | | | | | | | | | | | | | | | | | | | | | | | | | | |
| | Compounds | | | | | | | | | | | | | | | | | | | | | | | | | | |
| | 169 | 170 | 171 | 179 | 180 | 181 | 182 | 183 | 184 | 185 | 186 | 187 | 188 | 189 | 190 | 191 | 192 | 193 | 194 | 195 | 196 | 197 | 198 | 199 | 200 | 201 | 202 |
| Barnyardgrass | 0 | 10 | 0 | 0 | 20 | 80 | 40 | 60 | 0 | 0 | 30 | 60 | 60 | 60 | 20 | 20 | 80 | 80 | 50 | 60 | 20 | 0 | 20 | 10 | 0 | 0 | 40 |
| Ducksalad | 0 | 0 | 0 | 0 | 20 | 90 | 60 | 70 | 0 | 0 | 90 | 90 | 80 | 70 | 60 | 20 | 80 | 90 | 90 | 40 | 30 | 0 | 70 | 0 | 0 | 0 | 60 |
| Rice | 0 | 20 | 0 | 0 | 0 | 80 | 0 | 0 | 0 | 0 | 30 | 30 | 70 | 40 | 0 | 0 | 70 | 80 | 60 | 30 | 0 | 0 | 20 | 20 | 0 | 0 | 30 |
| Sedge, Umbrella | 0 | 20 | 0 | 0 | 0 | 80 | 80 | 80 | 30 | 60 | 90 | 80 | 80 | 70 | 60 | 90 | 80 | 80 | 80 | 50 | 30 | 0 | 70 | 10 | 50 | 0 | 50 |
| | 1000 g ai/ha | | | | | | | | | | | | | | | | | | | | | | | | | | |
| | Compounds | | | | | | | | | | | | | | | | | | | | | | | | | | |
| | 203 | 204 | 205 | 206 | 207 | 209 | 210 | 211 | 212 | 213 | 214 | 215 | 216 | 217 | 218 | 219 | 220 | 221 | 222 | 223 | 224 | 225 | 226 | 227 | 228 | 229 | 230 |
| Barnyardgrass | 0 | 20 | 70 | 10 | 80 | 60 | 80 | 20 | 50 | 60 | 0 | 30 | 20 | 0 | 0 | 0 | 0 | 40 | 90 | 40 | 90 | 90 | 80 | 70 | 90 | 90 | 50 |
| Ducksalad | 0 | 40 | 50 | 40 | 90 | 80 | 80 | 80 | 80 | 70 | 30 | 90 | 80 | 0 | 0 | 80 | 40 | 50 | 80 | 90 | 80 | 90 | 70 | 80 | 90 | 90 | 60 |
| Rice | 0 | 30 | 60 | 30 | 60 | 50 | 70 | 20 | 70 | 40 | 0 | 30 | 30 | 0 | 0 | 0 | 0 | 20 | 90 | 70 | 90 | 90 | 80 | 70 | 90 | 90 | 60 |
| Sedge, Umbrella | 0 | 80 | 60 | 50 | 80 | 90 | 80 | 30 | 70 | 80 | 50 | 90 | 90 | 0 | 20 | 80 | 80 | 80 | 70 | 80 | 80 | 90 | 80 | 50 | 80 | 80 | 70 |
| | 1000 g ai/ha | | | | | | | | | | | | | | | | | | | | | | | | | | |
| | Compounds | | | | | | | | | | | | | | | | | | | | | | | | | | |
| | 231 | 232 | 233 | 234 | 235 | 236 | 237 | 238 | 239 | 240 | 241 | 242 | 243 | 244 | 245 | 246 | 247 | 248 | 249 | 250 | 251 | 252 | 253 | 254 | 255 | 256 | 257 |
| Barnyardgrass | 80 | 40 | 0 | 0 | 40 | 80 | 60 | 10 | 0 | 0 | 80 | 90 | 90 | 90 | 90 | 50 | 70 | 40 | 0 | 0 | 0 | 70 | 0 | 30 | 70 | 30 | 0 |
| Ducksalad | 90 | 80 | 50 | 0 | 90 | 90 | 80 | 0 | 0 | 0 | 70 | 90 | 90 | 90 | 80 | 50 | 80 | 30 | 0 | 40 | 30 | 90 | 60 | 90 | 80 | 90 | 50 |
| Rice | 80 | 50 | 30 | 0 | 40 | 70 | 50 | 30 | 30 | 0 | 70 | 90 | 90 | 90 | 80 | 60 | 80 | 50 | 0 | 50 | 40 | 80 | 50 | 80 | 70 | 50 | 30 |
| Sedge, Umbrella | 90 | 90 | 80 | 0 | 80 | 80 | 40 | 80 | 70 | 60 | 80 | 90 | 80 | 80 | 50 | 50 | 70 | 50 | 30 | 70 | 40 | 80 | 20 | 60 | 80 | 70 | 0 |
| | 1000 g ai/ha | | | | | | | | | | | | | | | | | | | | | | | | | | |
| | Compounds | | | | | | | | | | | | | | | | | | | | | | | | | | |
| | 258 | 259 | 260 | 261 | 262 | 263 | 264 | 265 | 266 | 267 | 268 | 269 | 270 | 271 | 272 | 273 | 274 | 275 | 276 | 277 | 278 | 279 | 280 | 281 | 282 | 283 | 284 |
| Barnyardgrass | 70 | 30 | 60 | 0 | 0 | 60 | 0 | 0 | 80 | 60 | 90 | 90 | 50 | 0 | 50 | 60 | 70 | 20 | 70 | 0 | 60 | 10 | 0 | 70 | 20 | 20 | 30 |
| Ducksalad | 90 | 90 | 90 | 80 | 0 | 90 | 90 | 0 | 90 | 0 | 90 | 80 | 70 | 40 | 80 | 90 | 70 | 60 | 80 | 0 | 80 | 60 | 20 | 80 | 80 | 70 | 70 |
| Rice | 80 | 70 | 70 | 60 | 0 | 70 | 0 | 0 | 80 | 70 | 90 | 80 | 70 | 20 | 40 | 40 | 60 | 20 | 70 | 0 | 60 | 40 | 0 | 70 | 20 | 10 | 10 |
| Sedge, Umbrella | 70 | 70 | 70 | 30 | 0 | 80 | 50 | 0 | 80 | 70 | 80 | 80 | 60 | 40 | 80 | 80 | 80 | 80 | 80 | 0 | 70 | 60 | 30 | 70 | 0 | 80 | 0 |
| | 1000 g ai/ha | | | | | | | | | | | | | | | | | | | | | | | | | | |
| | Compounds | | | | | | | | | | | | | | | | | | | | | | | | | | |
| | 285 | 286 | 287 | 288 | 289 | 290 | 291 | 292 | 293 | 294 | 295 | 296 | 297 | 298 | 299 | 300 | 301 | 302 | 303 | 304 | 305 | 306 | 307 | 308 | 309 | 310 | 311 |
| Barnyardgrass | 90 | 90 | 80 | 0 | 0 | 70 | 0 | 20 | 90 | 90 | 60 | 80 | 30 | 90 | 90 | 90 | 90 | 70 | 90 | 80 | 90 | 80 | 90 | 90 | 90 | 90 | 50 |
| Ducksalad | 90 | 90 | 80 | 0 | 0 | 70 | 0 | 0 | 80 | 90 | 70 | 80 | 30 | 90 | 90 | 90 | 90 | 90 | 90 | 90 | 80 | 80 | 80 | 90 | 90 | 90 | 60 |
| Rice | 80 | 70 | 70 | 0 | 0 | 60 | 0 | 30 | 90 | 90 | 50 | 60 | 20 | 80 | 90 | 90 | 90 | 70 | 90 | 90 | 70 | 60 | 90 | 70 | 80 | 80 | 60 |
| Sedge, Umbrella | 90 | 80 | 80 | 60 | 0 | 70 | 0 | 60 | 80 | 80 | 80 | 80 | 80 | 70 | 90 | 90 | 90 | 80 | 90 | 90 | 90 | 80 | 90 | 90 | 80 | 80 | 30 |
| | 1000 g ai/ha | | | | | | | | | | | | | | | | | | | | | | | | | | |
| | Compounds | | | | | | | | | | | | | | | | | | | | | | | | | | |
| | 312 | 313 | 314 | 315 | 316 | 317 | 318 | 319 | 329 | 330 | 331 | 332 | 333 | 334 | 335 | 336 | 338 | 340 | 341 | 342 | 343 | 344 | 345 | 346 | 347 | 348 | 349 |
| Barnyardgrass | 0 | 70 | 30 | 0 | 0 | 0 | 70 | 10 | 0 | 0 | 80 | 60 | 50 | 90 | 60 | 90 | 40 | 90 | 90 | 90 | 90 | 60 | 30 | 40 | 0 | 0 | 80 |
| Ducksalad | 0 | 80 | 0 | 0 | 0 | 0 | 70 | 90 | 0 | 0 | 90 | 90 | 80 | 90 | 90 | 90 | 60 | 90 | 90 | 80 | 90 | 80 | 70 | 60 | 0 | 0 | 90 |
| Rice | 0 | 80 | 0 | 0 | 0 | 0 | 40 | 70 | 0 | 0 | 60 | 50 | 20 | 80 | 70 | 80 | 30 | 80 | 70 | 70 | 80 | 70 | 40 | 40 | 0 | 0 | 70 |
| Sedge, Umbrella | 0 | 80 | 60 | 0 | 0 | 0 | 80 | 80 | 0 | 0 | 90 | 90 | 80 | 80 | 80 | 80 | 80 | 80 | 80 | 70 | 90 | 80 | 80 | 80 | 0 | 0 | 80 |
| | 1000 g ai/ha | | | | | | | | | | | | | | | | | | | | | | | | | | |
| | Compounds | | | | | | | | | | | | | | | | | | | | | | | | | | |
| | 350 | 351 | 352 | 353 | 354 | 355 | 356 | 357 | 358 | 359 | 360 | 361 | 362 | 363 | 364 | 365 | 366 | 368 | 369 | 370 | 371 | 372 | 373 | 374 | 375 | 376 | 377 |
| Barnyardgrass | 0 | 0 | 90 | 70 | 40 | 50 | 50 | 70 | 80 | 70 | 40 | 80 | 50 | 60 | 90 | 90 | 80 | 60 | 50 | 0 | 30 | 90 | 20 | 40 | 40 | 70 | 30 |
| Ducksalad | 0 | 0 | 90 | 80 | 50 | 70 | 60 | 80 | 90 | 70 | 70 | 90 | 90 | 90 | 100 | 90 | 100 | 90 | 90 | 60 | 90 | 90 | 0 | 50 | 20 | 90 | 40 |
| Rice | 0 | 0 | 70 | 40 | 50 | 50 | 50 | 70 | 70 | 70 | 50 | 90 | 60 | 60 | 90 | 90 | 70 | 50 | 60 | 30 | 40 | 90 | 0 | 20 | 40 | 60 | 40 |
| Sedge, Umbrella | 0 | 0 | 80 | 90 | 50 | 70 | 70 | 80 | 80 | 80 | 90 | 90 | 90 | 80 | 100 | 90 | 90 | 80 | 80 | 40 | 60 | 90 | 20 | 0 | 80 | 80 | 80 |
| | 1000 g ai/ha | | | | | | | | | | | | | | | | | | | | | | | | | | |
| | Compounds | | | | | | | | | | | | | | | | | | | | | | | | | | |
| | 378 | 379 | 380 | 381 | 382 | 383 | 384 | 385 | 386 | 387 | 388 | 389 | 390 | 391 | 392 | 393 | 394 | 395 | 396 | 397 | 398 | 399 | 400 | 401 | 402 | 403 | 404 |
| Barnyardgrass | 80 | 40 | 70 | 10 | 80 | 30 | 90 | 80 | 90 | 0 | 70 | 0 | 0 | 90 | 90 | 90 | 70 | 90 | 90 | 0 | 0 | 90 | 0 | 0 | 10 | 0 | 90 |
| Ducksalad | 90 | 70 | 80 | 70 | 80 | 70 | 80 | 80 | 90 | 0 | 80 | 0 | 40 | 90 | 80 | 90 | 80 | 90 | 70 | 0 | 0 | 90 | 20 | 0 | 20 | 0 | 90 |
| Rice | 70 | 50 | 70 | 60 | 80 | 60 | 90 | 80 | 80 | 0 | 70 | 20 | 30 | 90 | 80 | 80 | 60 | 80 | 70 | 10 | 10 | 80 | 0 | 20 | 10 | 0 | 60 |
| Sedge, Umbrella | 80 | 70 | 70 | 70 | 70 | 80 | 80 | 80 | 80 | 0 | 70 | 30 | 60 | 80 | 80 | 90 | 90 | 90 | 80 | 70 | 80 | 80 | 80 | 0 | 70 | 10 | 90 |
| | 1000 g ai/ha | | | | | | | | | | | | | | | | | | | | | | | | | | |
| | Compounds | | | | | | | | | | | | | | | | | | | | | | | | | | |
| | 405 | 406 | 407 | 408 | 409 | 410 | 411 | 412 | 413 | 414 | 415 | 416 | 417 | 418 | 419 | 420 | 421 | 422 | 423 | 424 | 425 | 426 | 427 | 428 | 429 | 430 | 431 |
| Barnyardgrass | 40 | 90 | 80 | 70 | 90 | 80 | 50 | 40 | 60 | 80 | 60 | 60 | 70 | 80 | 90 | 90 | 0 | 30 | 80 | 40 | 30 | 0 | 0 | 0 | 100 | 80 | 60 |
| Ducksalad | 80 | 90 | 80 | 90 | 70 | 80 | 80 | 80 | 80 | 90 | 80 | 90 | 80 | 70 | 70 | 80 | 0 | 20 | 50 | 70 | 70 | 70 | 0 | 0 | 60 | 80 | 80 |
| Rice | 50 | 60 | 50 | 80 | 70 | 60 | 50 | 40 | 60 | 80 | 50 | 70 | 70 | 60 | 80 | 60 | 0 | 30 | 60 | 20 | 20 | 0 | 0 | 30 | 70 | 60 | 30 |
| Sedge, Umbrella | 90 | 90 | 90 | 90 | 90 | 80 | 80 | 90 | 90 | 90 | 80 | 80 | 70 | 80 | 80 | 70 | 0 | 70 | 70 | 70 | 70 | 70 | 0 | 0 | 90 | 80 | 80 |

| | 500 g ai/ha | | | | | 125 g ai/ha | | | | | | | | | | | | | | | | | | |
|---|---|---|---|---|---|---|---|---|---|---|---|---|---|---|---|---|---|---|---|---|---|---|---|---|
| | Compounds | | | | | Compounds | | | | | | | | | | | | | | | | | | |
| | 446 | 448 | 449 | 450 | 454 | 432 | 433 | 434 | 435 | 436 | 437 | 438 | 439 | 440 | 441 | 442 | 443 | 444 | 445 | 446 | 448 | 449 | 450 | 454 |
| Barnyardgrass | 90 | 100 | 30 | 80 | 90 | 30 | 30 | 60 | 0 | 0 | 0 | 0 | 0 | 0 | 0 | 0 | 80 | 0 | 90 | 70 | 90 | 0 | 50 | 50 |
| Ducksalad | 80 | 80 | 70 | 80 | 70 | 30 | 60 | 70 | 0 | 0 | 0 | 0 | 0 | — | 0 | 10 | 70 | 0 | 80 | 70 | 70 | 20 | 40 | 50 |
| Rice | 90 | 90 | 50 | 80 | 80 | 0 | 0 | 70 | 0 | 0 | 0 | 0 | 0 | 0 | 0 | 30 | 70 | 0 | 80 | 80 | 80 | 10 | 60 | 60 |
| Sedge, Umbrella | 90 | 90 | 80 | 80 | 70 | 80 | 70 | 90 | 0 | 0 | 0 | 0 | 0 | 0 | 0 | 70 | 80 | 0 | 80 | 90 | 90 | 30 | 60 | 60 |

Postemergence

| | 500 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 |
| Barnyardgrass | 30 | 70 | 20 | 30 | 20 | 80 | 0 | 0 | 40 | 10 | 0 | 0 | 10 | 20 | 10 | 60 | 10 | 10 | 0 | 10 | 0 | 0 | 0 | 0 | 0 | 10 | 60 |
| Blackgrass | 40 | 30 | 20 | 10 | 0 | 90 | 0 | 0 | 50 | 50 | 40 | 30 | 30 | 60 | 20 | 70 | 50 | 30 | 40 | 40 | 30 | 30 | 30 | 30 | 40 | 0 | 80 |
| Cocklebur | 90 | 80 | 70 | 70 | 70 | 80 | 0 | 0 | 70 | 80 | 10 | — | 50 | 20 | 20 | 20 | 70 | 20 | 50 | 50 | — | — | 10 | 40 | 0 | 0 | 50 |
| Corn | 20 | 20 | 20 | 20 | 20 | 60 | 10 | 0 | 10 | 0 | 0 | 0 | 0 | 0 | 0 | 30 | 30 | 20 | 20 | 20 | 0 | 0 | 20 | 0 | 0 | 10 | 40 |
| Crabgrass, Large | 0 | 30 | 10 | 30 | 20 | 30 | 0 | 0 | 20 | 20 | 10 | 0 | 30 | 30 | 10 | 10 | 10 | 10 | 10 | 10 | 10 | 10 | 0 | 10 | 0 | 10 | 80 |
| Foxtail, Giant | 30 | 30 | 20 | 30 | 30 | 20 | 0 | 0 | 30 | 20 | 30 | 30 | 30 | 40 | 40 | 30 | 40 | 30 | 20 | 20 | 20 | 20 | 0 | 20 | 0 | 10 | 100 |
| Lambsquarters | 80 | 90 | 60 | 100 | 80 | 100 | 20 | 10 | 80 | 90 | 70 | 70 | 70 | 70 | 70 | 70 | 90 | 50 | 50 | 40 | 40 | 60 | 20 | 10 | 0 | 10 | 100 |
| Morningglory | 60 | 70 | 50 | 50 | 20 | 0 | 0 | 0 | 0 | 20 | 10 | 10 | 10 | 20 | 0 | 20 | 10 | 20 | 10 | 40 | 20 | 30 | 30 | 20 | 20 | 0 | 50 |
| Oat, Wild | 50 | 30 | 20 | 20 | 20 | 70 | 0 | 0 | 50 | 30 | 20 | 10 | 30 | 40 | 30 | 40 | 30 | 30 | 30 | 40 | 30 | 10 | 20 | 30 | 30 | 0 | 60 |
| Pigweed | 70 | 70 | 40 | 80 | 60 | 70 | 0 | 10 | 80 | 90 | 20 | 30 | 70 | 60 | 30 | 60 | 60 | 40 | 60 | 60 | 30 | 20 | 20 | 30 | 10 | 50 | 100 |
| Surinam Grass | 20 | 50 | 10 | 30 | 20 | 60 | 0 | 0 | 0 | 30 | 10 | 20 | 20 | 20 | 10 | 20 | 20 | 10 | 20 | 10 | 10 | 0 | 10 | 20 | 0 | 10 | 80 |
| Velvetleaf | 80 | 50 | 10 | 20 | 20 | 60 | 0 | 0 | 70 | 70 | 20 | 20 | 50 | 50 | 30 | 60 | 60 | 20 | 10 | 30 | 20 | 30 | 0 | 10 | 0 | 0 | 70 |
| Wheat | 10 | 20 | 10 | 10 | 10 | 40 | 0 | 0 | 20 | 20 | 10 | 10 | 10 | 20 | 10 | 20 | 30 | 20 | 10 | 20 | 20 | 0 | 0 | 20 | 10 | 0 | 20 |
| | 500 g ai/ha | | | | | | | | | | | | | | | | | | | | | | | | | | |
| | Compounds | | | | | | | | | | | | | | | | | | | | | | | | | | |
| | 28 | 29 | 30 | 31 | 32 | 33 | 34 | 35 | 36 | 37 | 40 | 41 | 42 | 43 | 44 | 45 | 46 | 47 | 48 | 50 | 51 | 52 | 55 | 56 | 57 | 58 | 59 |
| Barnyardgrass | 40 | 50 | 10 | 50 | 10 | 30 | 30 | 20 | 20 | 20 | 10 | 20 | 20 | 70 | 60 | 30 | 50 | 40 | 60 | 70 | 80 | 0 | 10 | 20 | 20 | 60 | 20 |
| Blackgrass | 30 | 20 | 30 | 50 | 0 | 20 | 20 | 30 | 20 | 10 | 20 | 20 | 10 | 20 | 20 | 20 | 20 | 10 | 10 | 30 | 0 | 0 | 10 | 10 | 10 | 30 | 0 |
| Cocklebur | 80 | 80 | 70 | 90 | 20 | 100 | 90 | 100 | 50 | 80 | 50 | 40 | 20 | 90 | 80 | 80 | 90 | 100 | 70 | 90 | 80 | 40 | 40 | 80 | 60 | 100 | 40 |
| Corn | 20 | 20 | 20 | 30 | 10 | — | — | — | — | — | — | — | 20 | 30 | 30 | 30 | 30 | 30 | 30 | 40 | 0 | 40 | 30 | 30 | 20 | 20 | 20 |
| Crabgrass, Large | 30 | 70 | 10 | 30 | 10 | 30 | 20 | 20 | 20 | 20 | 20 | 30 | 70 | 90 | 90 | 30 | 90 | 70 | 20 | 30 | 50 | 10 | 10 | 20 | 20 | 30 | 20 |
| Foxtail, Giant | 50 | 40 | 20 | 30 | 10 | 30 | 30 | 20 | 30 | 20 | 20 | 20 | 10 | 50 | 30 | 30 | 40 | 30 | 30 | 30 | 0 | 10 | 10 | 20 | 30 | 20 | 20 |
| Lambsquarters | 90 | 100 | 90 | 90 | 90 | 90 | 90 | 90 | 90 | 90 | 70 | 70 | 70 | 90 | 90 | 90 | 90 | 90 | 90 | 90 | 90 | 80 | 70 | 90 | 90 | 90 | 90 |
| Morningglory | 40 | 70 | 60 | 70 | 0 | — | — | — | — | — | — | — | 50 | 100 | 90 | 90 | 90 | 100 | 50 | 70 | 0 | 0 | 10 | 20 | 10 | 30 | 20 |
| Oat, Wild | 30 | 40 | 30 | 30 | 20 | 10 | 10 | 10 | 20 | 20 | 10 | 10 | 20 | 30 | 20 | 20 | 10 | 30 | 30 | 20 | 10 | 10 | 20 | 20 | 30 | 30 | 20 |
| Pigweed | 100 | 100 | 100 | 90 | 50 | 90 | 90 | 100 | 90 | 90 | 60 | 70 | 70 | 100 | 100 | 100 | 90 | 100 | 70 | 100 | 100 | 10 | 40 | 90 | 80 | 90 | 80 |
| Surinam Grass | 30 | 30 | 40 | 60 | 10 | 30 | 50 | 40 | 10 | 20 | 20 | 20 | 20 | 80 | 20 | 30 | 40 | 40 | 50 | 40 | 20 | 0 | 10 | 30 | 20 | 30 | 20 |
| Velvetleaf | 60 | 70 | 50 | 50 | 30 | 60 | 50 | 50 | 40 | 40 | 20 | 20 | 60 | 90 | 80 | 70 | 80 | 70 | 80 | 60 | 70 | 20 | 40 | 50 | 30 | 70 | 20 |
| Wheat | 20 | 10 | 10 | 10 | 10 | 30 | 30 | 20 | 30 | 20 | 30 | 10 | 20 | 30 | 20 | 30 | 30 | 30 | 20 | 30 | 20 | 0 | 10 | 20 | 10 | 20 | 10 |
| | 500 g ai/ha | | | | | | | | | | | | | | | | | | | | | | | | | | |
| | Compounds | | | | | | | | | | | | | | | | | | | | | | | | | | |
| | 60 | 61 | 62 | 63 | 64 | 65 | 66 | 67 | 68 | 69 | 70 | 71 | 75 | 76 | 77 | 78 | 79 | 80 | 81 | 82 | 83 | 84 | 85 | 86 | 87 | 88 | 89 |
| Barnyardgrass | 10 | 30 | 50 | 20 | 10 | 10 | 10 | 10 | 0 | 0 | 20 | 10 | 20 | 20 | 0 | 30 | 40 | 90 | 90 | 20 | 50 | 40 | 80 | 80 | 0 | 60 | 20 |
| Blackgrass | 0 | 0 | 70 | 20 | 10 | 10 | 20 | 10 | 20 | 30 | 20 | 0 | 30 | 50 | 30 | 30 | 30 | 40 | 20 | 10 | 0 | 10 | 20 | 20 | 0 | 0 | 10 |
| Cocklebur | 70 | 100 | 100 | 70 | 50 | 80 | 30 | 70 | 10 | 0 | 100 | 0 | 30 | 90 | 0 | 100 | 80 | 100 | 90 | 100 | 20 | 100 | 100 | 100 | 0 | 50 | 90 |
| Corn | 20 | 40 | 20 | 20 | 20 | 20 | 20 | 10 | 0 | 0 | 20 | 10 | 20 | 30 | 10 | — | — | 50 | 30 | 30 | 30 | 20 | 30 | 30 | 0 | 70 | 20 |
| Crabgrass, Large | 20 | 30 | 20 | 20 | 10 | 20 | 10 | 10 | 0 | 0 | 20 | 10 | 10 | 20 | 40 | 30 | 30 | 100 | 20 | 20 | 30 | 50 | 80 | 90 | 60 | 60 | 20 |
| Foxtail, Giant | 20 | 30 | 30 | 20 | 10 | 20 | 20 | 10 | 0 | 0 | 30 | 10 | 20 | 40 | 40 | 30 | 40 | 90 | 50 | 10 | 10 | 10 | 50 | 60 | 0 | 40 | 20 |
| Lambsquarters | 90 | 100 | 90 | 80 | 70 | 70 | 70 | 60 | 10 | 10 | 80 | 20 | 40 | 60 | 90 | 80 | 80 | 80 | 50 | 50 | 60 | 60 | 60 | 90 | 0 | 100 | 60 |
| Morningglory | 20 | 50 | 60 | 20 | 20 | 20 | 20 | 10 | 0 | 0 | 30 | 0 | 50 | 10 | 70 | — | — | 100 | 30 | 70 | 30 | 60 | 100 | 100 | 0 | 0 | 50 |
| Oat, Wild | 20 | 20 | 30 | 30 | 20 | 20 | 10 | 30 | 20 | 20 | 20 | 0 | 40 | 40 | 40 | 20 | 30 | 50 | 30 | 30 | 20 | 20 | 50 | 60 | 0 | 0 | 30 |
| Pigweed | 70 | 60 | 90 | 70 | 50 | 60 | 30 | 20 | 30 | 20 | 50 | 20 | 70 | 100 | 90 | 90 | 100 | 100 | 90 | 100 | 100 | 100 | 100 | 100 | 0 | 60 | 40 |
| Surinam Grass | 20 | 30 | 70 | 20 | 10 | 20 | 10 | 10 | 0 | 0 | 20 | 10 | 20 | 20 | 20 | 20 | 40 | 90 | 60 | 60 | 40 | 60 | 70 | 100 | 0 | 0 | 20 |
| Velvetleaf | 60 | 70 | 70 | 50 | 10 | 10 | 10 | 10 | 0 | 0 | 60 | 0 | 20 | 40 | 30 | 80 | 80 | 100 | 90 | 90 | 70 | 100 | 80 | 70 | 0 | 70 | 50 |
| Wheat | 0 | 10 | 10 | 10 | 10 | 10 | 10 | 10 | 0 | 0 | 20 | 0 | 20 | 30 | 20 | 30 | 40 | 30 | 40 | 20 | 30 | 30 | 60 | 40 | 0 | 0 | 20 |
| | 500 g ai/ha | | | | | | | | | | | | | | | | | | | | | | | | | | |
| | Compounds | | | | | | | | | | | | | | | | | | | | | | | | | | |
| | 90 | 91 | 92 | 93 | 94 | 95 | 96 | 97 | 98 | 99 | 100 | 101 | 102 | 104 | 105 | 106 | 107 | 108 | 109 | 110 | 112 | 113 | 114 | 115 | 117 | 118 | 119 |
| Barnyardgrass | 30 | 0 | 0 | 10 | 10 | 20 | 50 | 0 | 10 | 0 | 0 | 20 | 40 | 0 | 0 | 0 | 0 | 70 | 20 | 20 | 70 | 80 | 30 | 50 | 30 | 30 | 10 |
| Blackgrass | 10 | 0 | 0 | 10 | 10 | 0 | 20 | 0 | 0 | 40 | 30 | 40 | 20 | 0 | 0 | 0 | 0 | 20 | 20 | 0 | 20 | 20 | 30 | 20 | 10 | 10 | 10 |
| Cocklebur | 60 | 10 | 0 | 50 | 50 | 70 | 100 | 70 | 20 | 30 | 0 | 80 | 50 | 0 | 30 | 50 | 0 | 90 | 70 | 40 | 100 | 100 | 90 | 90 | 90 | 90 | 60 |
| Corn | 10 | 10 | 10 | 20 | 20 | 10 | 20 | 0 | 10 | 20 | 0 | 40 | 40 | 0 | 0 | 10 | 0 | 20 | 20 | 20 | 40 | 30 | — | — | — | 40 | 30 |
| Crabgrass, Large | 10 | 10 | 10 | 10 | 10 | 50 | 50 | 0 | 20 | 10 | 0 | 20 | 70 | 0 | 0 | 10 | 0 | 70 | 10 | 20 | 30 | 80 | 40 | 30 | 30 | 30 | 20 |
| Foxtail, Giant | 10 | 0 | 0 | 10 | 10 | 30 | 50 | 0 | 10 | 30 | 0 | 30 | 30 | 0 | 0 | 0 | 0 | 30 | 20 | 10 | 30 | 40 | 30 | 30 | 20 | 20 | 10 |
| Lambsquarters | 50 | 30 | 20 | 90 | 80 | 100 | 100 | 70 | 30 | 90 | 50 | 90 | 20 | 20 | 80 | 90 | 20 | 100 | 20 | 60 | 100 | 100 | 90 | 100 | 90 | 20 | 60 |
| Morningglory | 40 | 0 | 0 | 40 | 10 | 60 | 0 | 0 | 20 | 20 | 0 | 10 | 30 | 0 | 10 | 10 | 0 | 100 | 10 | 30 | 30 | 100 | — | — | — | 70 | 40 |
| Oat, Wild | 30 | 10 | 0 | 10 | 10 | 20 | 20 | 0 | 10 | 40 | 40 | 50 | 40 | 0 | 10 | 20 | 0 | 50 | 30 | 0 | 40 | 60 | 10 | 30 | 20 | 20 | 20 |
| Pigweed | 40 | 10 | 10 | 30 | 30 | 100 | 100 | 0 | 30 | 70 | 60 | 90 | 100 | 10 | 80 | 20 | 10 | 100 | 70 | 50 | 100 | 100 | 100 | 100 | 90 | 100 | 30 |
| Surinam Grass | 10 | 10 | 10 | 10 | 10 | 20 | 30 | 0 | 20 | 10 | 20 | 20 | 100 | 10 | 10 | 10 | 10 | 10 | 20 | 30 | 20 | 60 | 20 | 60 | 40 | 40 | 10 |
| Velvetleaf | 30 | 10 | 0 | 10 | 20 | 60 | 80 | 60 | 10 | 40 | 0 | 30 | 20 | 0 | 0 | 20 | 0 | 70 | 20 | 40 | 70 | 60 | 60 | 80 | 50 | 40 | 50 |
| Wheat | 10 | 10 | 0 | 10 | 10 | 10 | 20 | 0 | 0 | 20 | 10 | 20 | 20 | 0 | 0 | 0 | 0 | 20 | 20 | 30 | 30 | 30 | 20 | 30 | 20 | 10 | 0 |
| | 500 g ai/ha | | | | | | | | | | | | | | | | | | | | | | | | | | |
| | Compounds | | | | | | | | | | | | | | | | | | | | | | | | | | |
| | 120 | 121 | 122 | 123 | 124 | 125 | 126 | 127 | 128 | 129 | 130 | 131 | 132 | 133 | 134 | 135 | 136 | 137 | 138 | 139 | 140 | 141 | 142 | 143 | 144 | 145 | 146 |
| Barnyardgrass | 10 | 0 | 20 | 0 | 0 | 0 | 30 | 20 | 30 | 0 | 80 | 50 | 60 | 40 | 40 | 40 | 90 | 40 | 20 | 10 | 0 | 10 | 10 | 0 | 80 | 40 | 60 |
| Blackgrass | 10 | 0 | 10 | 0 | 0 | 0 | 0 | 10 | 10 | 0 | 60 | 20 | 40 | 10 | 20 | 0 | 60 | 20 | 20 | 10 | 0 | 20 | 40 | 0 | 60 | 10 | 20 |
| Cocklebur | 60 | 20 | 90 | 0 | 10 | 0 | 100 | 80 | 100 | 60 | 100 | 100 | 90 | 60 | 100 | 90 | 100 | 100 | 90 | 100 | 20 | 20 | 40 | 0 | 100 | 30 | 80 |
| Corn | — | 10 | 10 | 0 | 10 | 0 | 20 | — | — | — | 40 | 20 | 20 | 20 | 30 | 30 | 30 | 20 | 30 | 20 | 10 | 10 | 0 | 10 | 30 | 20 | 30 |
| Crabgrass, Large | 20 | 10 | 10 | 0 | 10 | 0 | 40 | 30 | 20 | 10 | 90 | 30 | 40 | 30 | 30 | 30 | 70 | 30 | 20 | 20 | 0 | 30 | 10 | 0 | 100 | 10 | 20 |
| Foxtail, Giant | 10 | 10 | 20 | 0 | 10 | 0 | 30 | 30 | 20 | 10 | 60 | 30 | 40 | 20 | 40 | 40 | 10 | 20 | 30 | 20 | 0 | 20 | 30 | 0 | 90 | 40 | 20 |
| Lambsquarters | 50 | 80 | 0 | 0 | 70 | 20 | 60 | 80 | 60 | 10 | 100 | 90 | 100 | 70 | 90 | 80 | 100 | 80 | 90 | 90 | 80 | 90 | 80 | 20 | 90 | 70 | 90 |
| Morningglory | — | 0 | 0 | 0 | 10 | 0 | 30 | — | — | — | 100 | 60 | 70 | 20 | 40 | 50 | 100 | 60 | 60 | 100 | 0 | 30 | 50 | 0 | 80 | 20 | 20 |
| Oat, Wild | 10 | 30 | 40 | 0 | 10 | 0 | 50 | 10 | 10 | 0 | 60 | 20 | 20 | 20 | 30 | 20 | 60 | 70 | 30 | 10 | 0 | 20 | 20 | 20 | 50 | 30 | 20 |
| Pigweed | 70 | 50 | 10 | 0 | 70 | 0 | 40 | 60 | 60 | 10 | 100 | 60 | 90 | 50 | 90 | 90 | 90 | 100 | 90 | 90 | 0 | 80 | 60 | 20 | 100 | 100 | 90 |
| Surinam Grass | 50 | 30 | 10 | 0 | 20 | 10 | 20 | 10 | 20 | 20 | 90 | 60 | 60 | 50 | 40 | 40 | 70 | 70 | 30 | 20 | 0 | 20 | 20 | 10 | 90 | 20 | 30 |
| Velvetleaf | 30 | 20 | 20 | 0 | 10 | 0 | 40 | 10 | 60 | 20 | 70 | 40 | 60 | 50 | 40 | 30 | 70 | 60 | 50 | 60 | 0 | 80 | 60 | 20 | 100 | 60 | 60 |
| Wheat | 10 | 0 | 10 | 0 | 0 | 0 | 0 | 10 | 20 | 0 | 40 | 20 | 20 | 0 | 20 | 20 | 40 | 20 | 30 | 10 | 0 | 20 | 10 | 0 | 30 | 20 | 10 |
| | 500 g ai/ha | | | | | | | | | | | | | | | | | | | | | | | | | | |
| | Compounds | | | | | | | | | | | | | | | | | | | | | | | | | | |
| | 147 | 148 | 149 | 150 | 151 | 153 | 154 | 155 | 156 | 157 | 158 | 159 | 160 | 161 | 162 | 163 | 164 | 165 | 166 | 167 | 169 | 170 | 171 | 179 | 180 | 181 | 182 |
| Barnyardgrass | 30 | 20 | 30 | 50 | 10 | 30 | 50 | 20 | 40 | 20 | 0 | 0 | 10 | 10 | 30 | 0 | 0 | 0 | 0 | 0 | 10 | 10 | 10 | 0 | 0 | 10 | 50 |
| Blackgrass | 20 | 0 | 40 | 10 | 30 | 10 | 10 | 50 | 60 | 20 | 30 | — | 50 | 40 | 50 | 50 | 0 | 0 | 0 | 0 | 10 | 0 | 0 | 0 | 0 | 10 | 0 |
| Cocklebur | 80 | 70 | 100 | 80 | 0 | 100 | 70 | 80 | 80 | 90 | 70 | 80 | 90 | 30 | 60 | 60 | 0 | 0 | 0 | 0 | 0 | 0 | 0 | 0 | 0 | 70 | 60 |
| Corn | 30 | 10 | 30 | 50 | 10 | 20 | 20 | 20 | 10 | 0 | 10 | 0 | 0 | 0 | 0 | 0 | 10 | 0 | 0 | 0 | 10 | 10 | 10 | 0 | 0 | 20 | 30 |
| Crabgrass, Large | 70 | 10 | 30 | 10 | 0 | 20 | 20 | 40 | 10 | 20 | 30 | 0 | 0 | 10 | 40 | 40 | 0 | 0 | 0 | 0 | 10 | 10 | 10 | 0 | 0 | 20 | 30 |
| Foxtail, Giant | 40 | 10 | 10 | 10 | 20 | 20 | 20 | 30 | 40 | 40 | 30 | 50 | 30 | 30 | 30 | 40 | 0 | 0 | 0 | 0 | 0 | 10 | 20 | 0 | 0 | 20 | 20 |
| Lambsquarters | 90 | 10 | 60 | 70 | 50 | 80 | 90 | 90 | 80 | 70 | 80 | 90 | 80 | 70 | 90 | 80 | 10 | 0 | 0 | 0 | 60 | 70 | 20 | 0 | 0 | 90 | 70 |
| Morningglory | 30 | 10 | 40 | 40 | 30 | 70 | 70 | 60 | 50 | 30 | 50 | 10 | 70 | 20 | 60 | 0 | 0 | 0 | 0 | 0 | 70 | 80 | 0 | 0 | 0 | 20 | 70 |
| Oat, Wild | 30 | 10 | 30 | 20 | 30 | 50 | 30 | 50 | 40 | 40 | 40 | — | 30 | 40 | 40 | 30 | 0 | 0 | 0 | 0 | 10 | 10 | 0 | 0 | 0 | 30 | 30 |
| Pigweed | 90 | 20 | 70 | 90 | 30 | 70 | 80 | 100 | 60 | 80 | 70 | 80 | 80 | 60 | 80 | 80 | 0 | 0 | 0 | 0 | 40 | 50 | 20 | 0 | 0 | 90 | 70 |
| Surinam Grass | 30 | 10 | 20 | 40 | 10 | 30 | 60 | 30 | 20 | 20 | 0 | 20 | 10 | 20 | 40 | 40 | 10 | 0 | 0 | 0 | 10 | 10 | 10 | 0 | 0 | 20 | 20 |
| Velvetleaf | 60 | 10 | 50 | 60 | 20 | 60 | 60 | 70 | 90 | 80 | 50 | 70 | 70 | 50 | 80 | 80 | 0 | 0 | 0 | 0 | 0 | 20 | 0 | 0 | 0 | 20 | 60 |
| Wheat | 20 | 30 | 10 | 10 | 20 | 10 | 10 | 20 | 20 | 20 | 10 | — | 10 | 10 | 10 | 10 | 0 | 0 | 0 | 0 | 0 | 0 | 0 | 0 | 0 | 0 | 10 |
| | 500 g ai/ha | | | | | | | | | | | | | | | | | | | | | | | | | | |
| | Compounds | | | | | | | | | | | | | | | | | | | | | | | | | | |
| | 183 | 184 | 185 | 186 | 187 | 188 | 189 | 190 | 193 | 194 | 195 | 196 | 197 | 198 | 199 | 200 | 201 | 202 | 203 | 204 | 205 | 206 | 207 | 209 | 210 | 211 | 212 |
| Barnyardgrass | 0 | 0 | 50 | 50 | 10 | 20 | 10 | 10 | 10 | 10 | 90 | 30 | 10 | 30 | 10 | 10 | 0 | 0 | 0 | 10 | 20 | 10 | 20 | 20 | 40 | 10 | 20 |
| Blackgrass | 10 | 0 | 0 | 30 | 0 | 10 | 0 | 0 | 20 | 0 | 10 | 0 | 0 | 10 | 10 | 10 | 0 | 0 | 0 | 20 | 20 | 20 | 10 | 20 | 30 | 10 | 20 |
| Cocklebur | 90 | 60 | 50 | 90 | 70 | 90 | 10 | 10 | 70 | 10 | 60 | 20 | 10 | 90 | 60 | 20 | 10 | 40 | 10 | 90 | 80 | 40 | 80 | 40 | 90 | 60 | 90 |
| Corn | 20 | 30 | 30 | 20 | 20 | 20 | 10 | 10 | 20 | 0 | 30 | 10 | 20 | 70 | 20 | 30 | 20 | 10 | 10 | 20 | 20 | 20 | 20 | 30 | 60 | 10 | 40 |
| Crabgrass, Large | 30 | 30 | 40 | 10 | 10 | 10 | 20 | 20 | 10 | 10 | 50 | 30 | 0 | 20 | 10 | 10 | 10 | 10 | 10 | 10 | 20 | 30 | 20 | 20 | 20 | 10 | 10 |
| Foxtail, Giant | 30 | 40 | 30 | 20 | 10 | 10 | 10 | 10 | 20 | 0 | 20 | 0 | 0 | 30 | 10 | 10 | 0 | 10 | 10 | 20 | 30 | 20 | 20 | 30 | 30 | 10 | 40 |
| Lambsquarters | 80 | 70 | 70 | 30 | 0 | 70 | 50 | 80 | 90 | 30 | 60 | 30 | 0 | 70 | 70 | 60 | 40 | 50 | 50 | 50 | 70 | 80 | 80 | 40 | 80 | 70 | 100 |
| Morningglory | 70 | 60 | 70 | 30 | 20 | 60 | 90 | 90 | 70 | 10 | 60 | 0 | 0 | 80 | 60 | 40 | 30 | 40 | 30 | 40 | 50 | 20 | 20 | 20 | 70 | 30 | 50 |
| Oat, Wild | 20 | 30 | 30 | 20 | 20 | 20 | 10 | 20 | 0 | 0 | 20 | 0 | 30 | 30 | 10 | 10 | 10 | 10 | 10 | 20 | 30 | 20 | 20 | 20 | 20 | 10 | 20 |
| Pigweed | 80 | 60 | 80 | 60 | 20 | 70 | 60 | 70 | 60 | 30 | 80 | 30 | 10 | 50 | 40 | 40 | 40 | 40 | 10 | 70 | 50 | 40 | 60 | 40 | 70 | 30 | 90 |
| Surinam Grass | 20 | 20 | 30 | 30 | 10 | 10 | 20 | 20 | 10 | 10 | 30 | 40 | 20 | 20 | 20 | 20 | 10 | 10 | 20 | 50 | 20 | 20 | 20 | 20 | 30 | 10 | 60 |
| Velvetleaf | 60 | 60 | 50 | 60 | 10 | 20 | 20 | 40 | 70 | 10 | 40 | 0 | 20 | 20 | 30 | 30 | 0 | 30 | 10 | 70 | 40 | 20 | 30 | 20 | 70 | 30 | 70 |
| Wheat | 0 | 0 | 10 | 20 | 0 | 10 | 0 | 0 | 10 | 0 | 20 | 0 | 10 | 10 | 10 | 0 | 0 | 0 | 0 | 30 | 20 | 20 | 20 | 20 | 20 | 10 | 20 |
| | 500 g ai/ha | | | | | | | | | | | | | | | | | | | | | | | | | | |
| | Compounds | | | | | | | | | | | | | | | | | | | | | | | | | | |
| | 213 | 214 | 215 | 216 | 217 | 218 | 219 | 220 | 221 | 222 | 223 | 224 | 225 | 226 | 227 | 228 | 229 | 230 | 231 | 232 | 233 | 234 | 235 | 236 | 237 | 238 | 239 |
| Barnyardgrass | 50 | 0 | 20 | 10 | 0 | 0 | 0 | 0 | 10 | 20 | 20 | 30 | 30 | 20 | — | 70 | 50 | 20 | 40 | — | — | — | — | — | — | 0 | 0 |
| Blackgrass | 40 | 0 | 0 | 0 | 0 | 0 | 0 | 0 | 0 | 20 | 0 | 10 | 40 | 20 | 30 | 40 | 30 | 30 | 30 | 0 | 10 | 0 | 20 | 10 | 10 | 10 | 0 |
| Cocklebur | 70 | 0 | 80 | 70 | 0 | 0 | 20 | 10 | 20 | 60 | 30 | 40 | 20 | 20 | 30 | 80 | 70 | 10 | 40 | 30 | 20 | 20 | 30 | 40 | 40 | 20 | 20 |
| Corn | 20 | 20 | 10 | 10 | 0 | 10 | 10 | 10 | 10 | 20 | 20 | 20 | 20 | 40 | 40 | 30 | 20 | 20 | 20 | 20 | 30 | 20 | 40 | 40 | 40 | 0 | 20 |
| Crabgrass, Large | 30 | 0 | 0 | 0 | 0 | 10 | 10 | 10 | 10 | 20 | 20 | 20 | 20 | 20 | 30 | 40 | 20 | 20 | 20 | 20 | 10 | 20 | 20 | 20 | 20 | 0 | 20 |
| Foxtail, Giant | 30 | 20 | 20 | 20 | 0 | 0 | 10 | 10 | 10 | 30 | 20 | 30 | 20 | 20 | 60 | 50 | 50 | 20 | 30 | 30 | 30 | 30 | 30 | 40 | 20 | 0 | 0 |
| Lambsquarters | 40 | 0 | 0 | 0 | 0 | 10 | 10 | 20 | 20 | 70 | 80 | 70 | 70 | 60 | 70 | 80 | 80 | 60 | 70 | 60 | 70 | 40 | 50 | 70 | 70 | 0 | 30 |
| Morningglory | 30 | 0 | 40 | 0 | 10 | 10 | 60 | 0 | 0 | 80 | 80 | 70 | 40 | 80 | 60 | 80 | 70 | 70 | 70 | 70 | 80 | 70 | 70 | 60 | 60 | 0 | 10 |
| Oat, Wild | 20 | 0 | 20 | 0 | 0 | 0 | 0 | 10 | 10 | 20 | 30 | 30 | 30 | 30 | 0 | 50 | 50 | 40 | 40 | 0 | 20 | 20 | 20 | 20 | 20 | 10 | 0 |
| Pigweed | 20 | 0 | 70 | 20 | 0 | 20 | 20 | 10 | 20 | 60 | 70 | 60 | 70 | 60 | — | 80 | 80 | 60 | 70 | 40 | 70 | 30 | 40 | 70 | 60 | 0 | 50 |
| Surinam Grass | 20 | 10 | 10 | 20 | 10 | 10 | 10 | 10 | 10 | 30 | 10 | 20 | 20 | 20 | 30 | 40 | 20 | 10 | 30 | 20 | 20 | 10 | 10 | 20 | 20 | 0 | 30 |
| Velvetleaf | 60 | 10 | 60 | 60 | 10 | 0 | 10 | 0 | 0 | 60 | 20 | 60 | 40 | 20 | 70 | 80 | 70 | 30 | 50 | 40 | 50 | 30 | 30 | 50 | 50 | 10 | 10 |
| Wheat | 30 | 0 | 20 | 10 | 0 | 0 | 0 | 0 | 10 | 20 | 0 | 30 | 30 | 30 | 10 | 40 | 40 | 20 | 40 | 0 | 10 | 10 | 20 | 20 | 20 | 20 | 0 |
| | 500 g ai/ha | | | | | | | | | | | | | | | | | | | | | | | | | | |
| | Compounds | | | | | | | | | | | | | | | | | | | | | | | | | | |
| | 240 | 241 | 242 | 243 | 244 | 245 | 246 | 247 | 248 | 249 | 250 | 252 | 253 | 254 | 256 | 257 | 258 | 259 | 260 | 261 | 262 | 263 | 264 | 265 | 266 | 267 | 268 |
| Barnyardgrass | 0 | 0 | 60 | 20 | 50 | 70 | 70 | 10 | 40 | 20 | 20 | 10 | 10 | 10 | 60 | 0 | 0 | 0 | 0 | 0 | 10 | 10 | 0 | 0 | 0 | 90 | 30 |
| Blackgrass | 10 | 10 | 20 | 0 | 20 | 0 | 0 | 0 | 30 | 30 | 40 | 20 | 20 | 10 | 20 | 10 | 10 | 20 | 10 | 20 | 20 | 20 | 20 | 10 | 20 | 40 | 50 |
| Cocklebur | 20 | 30 | 90 | 30 | 80 | 80 | 30 | 30 | 50 | 30 | 40 | 40 | 20 | 20 | 0 | 70 | 30 | 20 | 20 | 10 | 10 | 10 | 0 | 0 | 0 | 90 | 50 |
| Corn | 10 | 10 | 40 | 10 | 30 | 30 | 10 | 20 | 30 | 20 | 30 | 10 | 0 | 10 | 10 | 0 | 10 | 20 | 0 | 20 | 20 | 10 | 0 | 10 | 0 | 40 | 30 |
| Crabgrass, Large | 20 | 10 | 50 | 10 | 30 | 50 | 20 | 10 | 20 | 30 | 20 | 10 | 0 | 10 | 20 | 10 | 0 | 20 | 10 | 10 | 20 | 20 | 0 | 0 | 0 | 40 | 20 |
| Foxtail, Giant | 0 | 0 | 30 | 20 | 30 | 60 | 30 | 20 | 30 | 30 | 30 | 30 | 0 | 0 | 50 | 0 | 0 | 0 | 0 | 0 | 20 | 20 | 0 | 0 | 20 | 50 | 20 |
| Lambsquarters | 50 | 50 | 90 | 80 | 70 | 90 | 70 | 70 | 70 | 20 | 40 | 30 | 30 | 20 | 60 | 50 | 70 | 30 | 30 | 60 | 80 | 80 | 0 | 0 | 100 | 50 | 70 |
| Morningglory | 30 | 70 | 40 | 20 | 20 | 60 | 10 | 30 | 50 | 30 | 30 | 20 | 30 | 30 | 10 | 0 | 10 | 0 | 10 | 0 | 0 | 20 | 0 | 10 | 50 | 40 | 20 |
| Oat, Wild | 0 | 20 | 30 | 20 | 50 | 30 | 20 | 10 | 60 | 40 | 40 | 20 | 30 | 20 | 30 | 30 | 30 | 20 | 30 | 20 | 0 | 20 | 20 | 10 | 20 | 40 | 50 |
| Pigweed | 50 | 50 | 80 | 60 | 70 | 80 | 60 | 60 | 80 | 80 | 70 | 30 | 30 | 30 | 60 | 80 | 60 | 90 | 70 | 70 | 80 | 90 | 0 | 20 | 100 | 90 | 50 |
| Surinam Grass | 20 | 20 | 60 | 20 | 30 | 60 | 20 | 20 | 20 | 30 | 20 | 20 | 0 | 10 | 20 | 20 | 10 | 20 | 20 | 20 | 20 | 10 | 0 | 0 | 20 | 50 | 50 |
| Velvetleaf | 10 | 10 | 60 | 60 | 60 | 80 | 60 | 70 | 30 | 20 | 30 | 30 | 10 | 10 | 30 | 10 | 0 | 0 | 20 | 0 | 20 | 20 | 0 | 10 | 0 | 70 | 60 |
| Wheat | 20 | 10 | 10 | 0 | 20 | 20 | 30 | 0 | 40 | 30 | 40 | 20 | 30 | 20 | 30 | 20 | 20 | 20 | 40 | 20 | 10 | 20 | 10 | 10 | 30 | 30 | 30 |
| | 500 g ai/ha | | | | | | | | | | | | | | | | | | | | | | | | | | |
| | Compounds | | | | | | | | | | | | | | | | | | | | | | | | | | |
| | 269 | 270 | 271 | 272 | 273 | 274 | 275 | 276 | 277 | 278 | 279 | 280 | 281 | 282 | 283 | 284 | 285 | 286 | 287 | 288 | 289 | 290 | 291 | 292 | 293 | 294 | 295 |
| Barnyardgrass | 30 | 0 | 0 | 80 | 70 | 10 | 20 | 10 | 0 | 10 | 10 | 0 | 0 | 20 | 0 | 0 | 10 | 0 | 20 | 0 | 0 | 0 | 30 | 0 | 10 | 60 | 50 |
| Blackgrass | 50 | 20 | 20 | 100 | 70 | 30 | 40 | 40 | 20 | 30 | 30 | 20 | 30 | 30 | 40 | 40 | 30 | 20 | 30 | 20 | 10 | 30 | 10 | 20 | 30 | 60 | 40 |
| Cocklebur | 50 | 0 | 0 | 90 | 80 | 10 | 10 | 10 | 0 | 30 | 20 | 30 | 20 | 20 | 0 | 0 | 30 | 20 | 10 | 0 | 0 | 10 | 10 | 10 | 30 | 30 | 90 |
| Corn | 30 | 10 | 20 | 40 | 20 | 20 | 0 | 20 | 10 | 20 | 10 | 10 | 10 | 20 | 10 | 10 | 10 | 10 | 70 | 0 | 0 | 10 | 60 | 20 | 30 | 50 | 20 |
| Crabgrass, Large | 20 | 0 | 0 | 70 | 50 | 0 | 10 | 20 | 0 | 10 | 10 | 0 | 10 | 10 | 10 | 10 | 20 | 10 | 0 | 0 | 0 | 0 | 30 | 10 | 20 | 20 | 60 |
| Foxtail, Giant | 30 | 0 | 0 | 30 | 20 | 50 | 0 | 40 | 0 | 10 | 0 | 0 | 10 | 20 | 0 | 0 | 50 | 50 | 80 | 30 | 60 | 60 | 80 | 20 | 30 | 50 | 70 |
| Lambsquarters | 70 | 10 | 60 | 100 | 90 | 70 | 70 | 60 | 30 | 30 | 20 | 20 | 30 | 60 | 30 | 50 | 70 | 70 | 20 | 30 | 30 | 20 | 60 | 70 | 70 | 80 | 60 |
| Morningglory | 40 | 10 | 0 | 100 | 70 | 60 | 10 | 30 | 20 | 20 | 20 | 10 | 10 | 20 | 30 | 0 | 30 | 0 | 20 | 0 | 0 | 0 | 40 | 20 | 70 | 20 | 100 |
| Oat, Wild | 30 | 0 | 30 | 90 | 50 | 40 | 40 | 40 | 20 | 30 | 20 | 20 | 20 | 20 | 10 | 30 | 50 | 40 | 40 | 40 | 30 | 40 | 20 | 40 | 50 | 50 | 50 |
| Pigweed | 60 | 0 | 80 | 100 | 90 | 60 | 70 | 70 | 10 | 40 | 30 | 20 | 30 | 60 | 20 | 70 | 80 | 100 | 30 | 20 | 20 | 10 | 30 | 30 | 40 | 70 | 100 |
| Surinam Grass | 20 | 10 | 0 | 60 | 40 | 30 | 30 | 20 | 0 | 10 | 10 | 10 | 20 | 10 | 10 | 20 | 30 | 20 | 10 | 0 | 0 | 30 | 60 | 20 | 20 | 40 | 60 |
| Velvetleaf | 20 | 0 | 0 | 100 | 70 | 10 | 30 | 40 | 20 | 10 | 10 | 20 | 20 | 20 | 0 | 0 | 40 | 60 | 60 | 20 | 10 | 10 | 60 | 10 | 30 | 80 | 100 |
| Wheat | 30 | 0 | 30 | 30 | 40 | 20 | 30 | 30 | 20 | 30 | 30 | 30 | 30 | 30 | 20 | 30 | 40 | 30 | 30 | 30 | 20 | 30 | 0 | 20 | 30 | 40 | 30 |
| | 500 g ai/ha | | | | | | | | | | | | | | | | | | | | | | | | | | |
| | Compounds | | | | | | | | | | | | | | | | | | | | | | | | | | |
| | 296 | 297 | 298 | 299 | 300 | 301 | 302 | 303 | 304 | 305 | 306 | 307 | 308 | 309 | 310 | 311 | 312 | 313 | 314 | 315 | 316 | 317 | 318 | 319 | 329 | 330 | 331 |
| Barnyardgrass | 80 | 20 | 50 | 90 | 50 | 60 | 40 | 40 | 20 | 0 | 10 | 10 | 10 | 40 | 20 | 0 | 0 | 50 | 30 | 20 | 40 | 0 | 70 | 20 | 10 | 0 | 20 |
| Blackgrass | 40 | 20 | 50 | 50 | 30 | 40 | 20 | 40 | 40 | 30 | 30 | 30 | 30 | 30 | 30 | 0 | 0 | 10 | 20 | 0 | 0 | 0 | 10 | 0 | 20 | 0 | 20 |
| Cocklebur | 60 | 0 | 50 | 70 | 50 | 50 | 10 | 30 | 100 | 10 | 70 | 30 | 40 | 70 | 20 | 0 | 0 | 90 | 30 | 20 | 20 | 0 | 80 | 40 | 30 | 0 | 30 |
| Corn | 70 | 100 | 10 | 20 | 30 | 50 | 50 | 70 | 80 | 70 | 30 | 10 | 10 | 60 | 20 | 0 | 0 | 20 | 20 | 20 | 20 | 0 | 20 | 20 | 20 | 0 | 0 |
| Crabgrass, Large | 60 | 30 | 70 | 90 | 30 | 60 | 10 | 60 | 10 | 0 | 50 | 30 | 10 | 40 | 30 | 0 | 0 | 20 | 20 | 10 | 10 | 0 | 60 | 20 | 10 | 0 | 10 |
| Foxtail, Giant | 60 | 30 | 50 | 80 | 30 | 20 | 20 | 50 | 30 | 20 | 40 | 30 | 20 | 40 | 40 | 0 | 0 | 50 | 20 | 10 | 20 | 0 | 10 | 30 | 30 | 0 | 30 |
| Lambsquarters | 70 | 20 | 90 | 100 | 80 | 80 | 70 | 90 | 90 | 80 | 80 | 80 | 60 | 80 | 80 | 0 | 20 | 90 | 60 | 60 | 70 | 50 | 80 | 60 | 70 | 10 | 70 |
| Morningglory | 80 | 30 | 70 | 80 | 90 | 90 | 60 | 90 | 100 | 0 | 70 | 0 | 20 | 40 | 50 | 0 | 20 | 80 | 80 | 20 | 10 | 0 | 80 | 20 | 20 | 10 | 10 |
| Oat, Wild | 40 | 20 | 40 | 40 | 30 | 40 | 10 | 40 | 40 | 30 | 30 | 20 | 20 | 40 | 40 | 0 | 0 | 30 | 30 | 10 | 20 | 0 | 30 | 20 | 30 | 20 | 30 |
| Pigweed | 100 | 40 | 90 | 100 | 90 | 90 | 90 | 90 | 90 | 70 | 100 | 60 | 70 | 70 | 70 | 0 | 20 | 70 | 70 | 50 | 50 | 0 | 100 | 40 | 80 | 0 | 50 |
| Surinam Grass | 50 | 30 | 50 | 80 | 10 | 30 | 10 | 20 | 10 | 20 | 30 | 30 | 20 | 40 | 20 | 0 | 0 | 20 | 20 | 20 | 10 | 0 | 20 | 20 | 20 | 0 | 20 |
| Velvetleaf | 100 | 10 | 60 | 100 | 70 | 100 | 10 | 70 | 60 | 20 | 60 | 60 | 20 | 40 | 50 | 0 | 0 | 70 | 30 | 20 | 40 | 0 | 80 | 20 | 30 | 10 | 20 |
| Wheat | 30 | 10 | 30 | 30 | 20 | 20 | 10 | 20 | 20 | 10 | 20 | 20 | 20 | 30 | 20 | 0 | 0 | 10 | 10 | 0 | 0 | 0 | 10 | 30 | 30 | 0 | 20 |
| | 500 g ai/ha | | | | | | | | | | | | | | | | | | | | | | | | | | |
| | Compounds | | | | | | | | | | | | | | | | | | | | | | | | | | |
| | 332 | 333 | 334 | 335 | 336 | 338 | 340 | 341 | 342 | 343 | 344 | 345 | 347 | 348 | 349 | 350 | 351 | 352 | 353 | 354 | 355 | 356 | 357 | 358 | 359 | 360 | 361 |
| Barnyardgrass | 60 | 40 | 60 | 50 | 30 | 40 | 20 | 20 | 20 | 50 | 10 | 0 | 0 | 0 | 10 | 0 | 0 | 10 | 10 | 0 | 10 | 0 | 10 | 20 | 10 | 30 | 10 |
| Blackgrass | 80 | 70 | 70 | 30 | 20 | 40 | 30 | 20 | 30 | 10 | 0 | 0 | 30 | 40 | 80 | 0 | 0 | 30 | 40 | 20 | 20 | 10 | 30 | 40 | 50 | 50 | 40 |
| Cocklebur | 70 | 80 | 70 | 70 | 50 | 10 | 20 | 20 | 20 | 20 | 10 | 0 | 0 | 0 | 20 | 20 | 0 | 10 | 20 | 20 | 10 | 0 | 20 | 20 | 30 | 20 | 20 |
| Corn | 10 | 10 | 20 | 20 | 20 | 20 | 20 | 10 | 30 | 30 | 10 | 0 | 0 | 0 | 10 | 20 | 10 | 10 | 20 | 0 | 0 | 0 | 20 | 0 | 10 | 20 | 0 |
| Crabgrass, Large | 80 | 100 | 50 | 40 | 50 | 60 | 20 | 20 | 20 | 50 | 10 | 0 | 0 | 0 | 20 | 10 | 10 | 20 | 10 | 10 | 10 | 30 | 10 | 10 | 20 | 20 | 10 |
| Foxtail, Giant | 50 | 40 | 60 | 40 | 40 | 30 | 40 | 30 | 50 | 50 | 10 | 0 | 0 | 0 | 30 | 30 | 0 | 30 | 30 | 20 | 30 | 30 | 40 | 30 | 20 | 50 | 30 |
| Lambsquarters | 100 | 100 | 90 | 30 | 90 | 50 | 70 | 100 | 100 | 100 | 40 | 20 | 10 | 0 | 80 | 10 | 10 | 60 | 40 | 50 | 70 | 50 | 60 | 50 | 50 | 20 | 70 |
| Morningglory | 70 | 60 | 80 | 40 | 70 | 100 | 40 | 40 | 80 | 80 | 50 | 10 | 10 | 0 | 50 | — | 0 | 60 | — | 60 | 40 | 10 | 30 | 50 | 10 | 10 | 80 |
| Oat, Wild | 60 | 60 | 30 | 40 | 20 | 40 | 30 | 30 | 30 | 10 | 0 | 10 | 20 | 30 | 40 | 0 | 0 | 20 | 40 | 20 | 30 | 20 | 30 | 30 | 30 | 60 | 20 |
| Pigweed | 100 | 100 | 70 | 70 | 70 | 80 | 80 | 60 | 80 | 90 | 50 | 20 | 10 | 0 | 70 | 60 | 50 | 60 | 70 | 50 | 50 | 70 | 40 | 40 | 50 | 40 | 40 |
| Surinam Grass | 40 | 30 | 50 | 30 | 20 | 40 | 20 | 20 | 20 | 40 | 10 | 0 | 0 | 0 | 20 | 10 | 0 | 40 | 10 | 20 | 20 | 20 | 20 | 10 | 10 | 30 | 10 |
| Velvetleaf | 90 | 60 | 60 | 60 | 30 | 50 | 40 | 30 | 50 | 50 | 30 | 10 | 0 | 0 | 40 | 10 | 0 | 60 | 20 | 10 | 50 | 0 | 20 | 20 | 20 | 50 | 30 |
| Wheat | 30 | 30 | 20 | 20 | 10 | 10 | 10 | 20 | 10 | 0 | 10 | 0 | 0 | 20 | 10 | 0 | 0 | 20 | 10 | 20 | 20 | 10 | 0 | 10 | 20 | 40 | 0 |
| | 500 g ai/ha | | | | | | | | | | | | | | | | | | | | | | | | | | |
| | Compounds | | | | | | | | | | | | | | | | | | | | | | | | | | |
| | 362 | 363 | 364 | 365 | 366 | 368 | 369 | 370 | 371 | 372 | 373 | 374 | 375 | 376 | 377 | 378 | 379 | 380 | 381 | 382 | 383 | 384 | 385 | 386 | 387 | 388 | 389 |
| Barnyardgrass | 0 | 0 | 70 | 10 | 10 | 10 | 20 | 0 | 0 | 60 | 20 | 20 | 70 | 40 | 0 | 0 | 0 | 0 | 0 | 0 | 0 | 30 | 10 | 10 | 0 | 10 | 0 |
| Blackgrass | 20 | 70 | 80 | 90 | 50 | 10 | 30 | 10 | 20 | 30 | 10 | 30 | 40 | 40 | 0 | 0 | 0 | 40 | 30 | 30 | 20 | 50 | 30 | 40 | 20 | 20 | 10 |
| Cocklebur | 30 | 30 | 50 | 60 | 50 | 30 | 70 | 0 | 30 | 30 | 30 | 30 | 60 | 30 | 0 | 0 | 10 | 30 | 20 | 10 | 10 | 40 | 50 | 50 | 20 | 20 | 0 |
| Corn | 0 | 0 | 40 | 20 | 20 | 10 | 10 | 10 | 10 | 60 | 20 | 20 | 40 | 30 | 10 | 10 | 0 | 20 | 10 | 0 | 0 | 10 | 10 | 10 | 0 | 20 | 0 |
| Crabgrass, Large | 0 | 10 | 70 | 80 | 20 | 10 | 20 | 10 | 10 | 20 | 10 | 20 | 40 | 50 | 0 | 10 | 10 | 10 | 20 | 10 | 10 | 40 | 20 | 20 | 0 | 20 | 10 |
| Foxtail, Giant | 10 | 10 | 50 | 70 | 40 | 10 | 30 | 20 | 20 | 50 | 30 | 20 | 70 | 60 | 10 | 30 | 10 | 20 | 20 | 10 | 0 | 30 | 20 | 20 | 20 | 30 | 10 |
| Lambsquarters | 30 | 70 | 90 | 100 | 90 | 60 | 70 | 20 | 70 | 90 | 30 | 70 | 40 | 90 | 70 | 70 | 50 | 70 | 80 | 70 | 30 | 80 | 60 | 60 | 20 | 70 | 30 |
| Morningglory | 80 | 70 | 80 | — | 100 | 30 | 20 | 20 | 70 | 60 | 20 | 20 | 40 | 60 | 60 | 30 | 40 | 60 | 80 | 60 | 10 | 40 | 80 | 20 | 10 | 50 | 0 |
| Oat, Wild | 40 | 40 | 50 | 50 | 50 | 0 | 30 | 0 | 10 | 30 | 30 | 40 | 50 | 40 | 0 | 20 | 0 | 30 | 20 | 20 | 20 | 30 | 20 | 0 | 30 | 10 | 0 |
| Pigweed | 50 | 30 | 90 | — | 80 | 50 | 60 | 30 | 30 | 80 | 70 | 70 | 80 | 90 | — | 70 | 50 | 80 | 30 | 40 | 30 | 70 | 60 | 60 | 40 | 50 | 40 |
| Surinam Grass | 10 | 20 | 70 | 20 | 20 | 0 | 10 | 10 | 10 | 10 | 20 | 20 | 30 | 20 | 10 | 20 | 20 | 30 | 40 | 10 | 20 | 60 | 20 | 10 | 0 | 20 | 10 |
| Velvetleaf | 0 | 20 | 50 | 40 | 60 | 20 | 60 | 20 | 20 | 30 | 30 | 20 | 90 | 40 | 40 | 40 | 10 | 30 | 40 | 20 | 40 | 70 | 20 | 50 | 10 | 40 | 20 |
| Wheat | 10 | 20 | 30 | 30 | 20 | 0 | 0 | 0 | 10 | 10 | 10 | 20 | 40 | 20 | 0 | 0 | 0 | 10 | 20 | 10 | 0 | 20 | 10 | 0 | 10 | 10 | 0 |
| | 500 g ai/ha | | | | | | | | | | | | | | | | | | | | | | | | | | |
| | Compounds | | | | | | | | | | | | | | | | | | | | | | | | | | |
| | 390 | 391 | 392 | 393 | 394 | 395 | 396 | 397 | 398 | 399 | 400 | 401 | 402 | 403 | 404 | 405 | 406 | 407 | 408 | 409 | 410 | 411 | 412 | 413 | 414 | 415 | 416 |
| Barnyardgrass | 10 | 30 | 20 | 20 | 10 | 70 | 20 | 0 | 0 | 40 | 0 | 0 | 0 | 0 | 10 | 10 | 20 | 10 | 40 | 0 | 0 | 30 | 0 | 20 | 30 | 20 | 30 |
| Blackgrass | 10 | 70 | 60 | 40 | 20 | 40 | 30 | 0 | 10 | 70 | 0 | 0 | 0 | 0 | 30 | 10 | 20 | 20 | 80 | 40 | 30 | 60 | 40 | 20 | 50 | 40 | 30 |
| Cocklebur | 20 | 30 | 40 | 60 | 20 | 80 | 60 | 0 | 10 | 80 | 0 | 0 | 0 | 0 | 30 | 20 | 50 | 20 | 70 | 10 | 10 | 60 | 30 | 90 | 70 | 50 | 60 |
| Corn | 20 | 30 | 20 | 20 | 10 | 60 | 10 | 10 | 20 | 40 | 0 | 0 | 0 | 0 | 10 | 10 | 10 | 20 | 10 | 10 | 10 | 10 | 0 | 20 | 40 | 20 | 30 |
| Crabgrass, Large | 20 | 30 | 20 | 20 | 20 | 50 | 20 | 10 | 10 | 30 | 0 | 0 | 10 | 0 | 10 | 20 | 20 | 20 | 50 | 10 | 20 | 30 | 30 | 20 | 70 | 50 | 40 |
| Foxtail, Giant | 30 | 30 | 30 | 30 | 30 | 50 | 30 | 40 | 30 | 50 | 0 | 0 | 20 | 0 | 40 | 20 | 40 | 30 | 50 | 20 | 20 | 30 | 30 | 20 | 40 | 40 | 30 |
| Lambsquarters | 60 | 90 | 90 | 80 | 60 | 100 | 100 | 0 | 10 | 100 | 10 | 0 | 40 | 10 | 80 | 70 | 60 | 60 | 100 | 70 | 70 | 90 | 100 | 90 | 100 | 80 | 80 |
| Morningglory | 40 | 70 | 70 | 50 | 20 | 80 | 60 | 10 | 20 | 40 | 0 | 0 | 0 | 0 | 70 | 60 | 60 | 60 | 30 | 70 | 80 | 0 | 0 | 70 | 70 | 40 | 70 |
| Oat, Wild | 10 | 20 | 30 | 30 | 0 | 40 | 30 | 0 | 0 | 40 | 0 | 0 | 0 | 0 | 10 | 10 | 10 | 0 | 50 | 10 | 10 | 50 | 40 | 10 | 40 | 40 | 40 |
| Pigweed | 60 | 70 | 60 | 50 | 60 | 60 | 70 | 0 | 10 | 80 | 20 | 0 | 10 | 0 | 30 | 50 | 50 | 40 | 100 | 70 | 70 | 90 | 100 | 70 | 100 | 90 | 80 |
| Surinam Grass | 10 | 30 | 20 | 10 | 20 | 60 | 30 | 10 | 10 | 40 | 0 | 0 | 20 | 10 | 10 | 10 | 20 | 20 | 30 | 30 | 0 | 10 | 30 | 20 | 60 | 30 | 30 |
| Velvetleaf | 20 | 60 | 40 | 60 | 30 | 60 | 50 | 50 | 60 | 80 | 30 | 0 | 30 | 10 | 30 | 30 | 30 | 20 | 70 | 0 | 0 | 10 | 10 | 50 | 70 | 50 | 50 |
| Wheat | 0 | 10 | 10 | 10 | 0 | 30 | 20 | 10 | 10 | 30 | 0 | 0 | 0 | 0 | 0 | 0 | 0 | 0 | 30 | 10 | 10 | 30 | 20 | 10 | 20 | 30 | 30 |
| | 500 g ai/ha | | | | | | | | | | | | | | | | | | | | | | | | | | |
| | Compounds | | | | | | | | | | | | | | | | | | | | | | | | | | |
| | 417 | 418 | 419 | 420 | 421 | 422 | 423 | 424 | 425 | 426 | 427 | 428 | 429 | 430 | 431 | 432 | 433 | 434 | 435 | 436 | 437 | 438 | 439 | 440 | 441 | 442 | 443 |
| Barnyardgrass | 20 | 20 | 50 | 50 | 0 | 0 | 30 | 30 | 20 | 20 | 0 | 10 | 60 | 50 | 20 | 60 | 20 | 30 | 10 | 0 | 30 | 0 | 0 | 0 | 0 | 20 | 30 |
| Blackgrass | 40 | 30 | 30 | 40 | 20 | 20 | 60 | 50 | 20 | 0 | 20 | 0 | 50 | 70 | 50 | 40 | 40 | 50 | 30 | 0 | 30 | 30 | 10 | 20 | 40 | 30 | 40 |
| Cocklebur | 40 | 30 | 40 | 40 | 0 | 0 | 30 | 30 | 20 | 20 | 10 | 10 | 70 | 90 | 60 | 70 | 90 | 70 | 20 | 10 | 30 | 20 | 0 | 0 | 10 | 100 | 80 |
| Corn | 30 | 20 | 30 | 20 | 0 | 0 | 20 | 10 | 20 | 10 | 0 | 10 | 40 | 40 | 20 | 30 | 30 | 40 | 30 | 10 | 30 | 40 | 0 | 20 | 30 | 30 | 20 |
| Crabgrass, Large | 40 | 30 | 40 | 40 | 0 | 0 | 50 | 30 | 30 | 20 | 30 | 20 | 40 | 50 | 30 | 30 | 30 | 30 | 30 | 0 | 20 | 0 | 0 | 10 | 0 | 20 | 30 |
| Foxtail, Giant | 40 | 40 | 30 | 30 | 0 | 0 | 30 | 30 | 30 | 30 | 10 | 10 | 40 | 40 | 30 | 40 | 40 | 30 | 30 | 0 | 30 | 20 | 0 | 0 | 0 | 20 | 40 |
| Lambsquarters | 80 | 90 | 90 | 90 | 0 | 0 | 70 | 70 | 70 | 50 | 40 | 40 | 70 | 80 | 60 | 30 | 80 | 70 | 60 | 0 | 40 | 10 | 0 | 10 | 20 | 70 | 80 |
| Morningglory | 60 | 80 | 90 | 90 | 0 | 0 | 80 | 70 | 80 | 50 | 30 | 30 | 80 | 70 | 70 | 30 | 80 | 70 | 90 | 0 | 30 | 50 | 30 | 20 | 0 | 30 | 60 |
| Oat, Wild | 40 | 10 | 20 | 30 | 0 | 0 | 30 | 0 | 0 | 0 | 0 | 0 | 40 | 50 | 50 | 60 | 60 | 60 | 60 | 30 | 30 | 40 | 10 | 30 | 40 | 60 | 60 |
| Pigweed | 80 | 80 | 80 | 70 | 0 | 0 | 60 | 60 | 70 | 50 | 40 | 30 | 50 | 80 | 70 | 10 | 70 | 60 | 60 | 10 | 30 | 30 | 10 | 10 | 20 | 70 | 80 |
| Surinam Grass | 30 | 20 | 30 | 30 | 0 | 0 | 30 | 30 | 20 | 20 | 10 | 20 | 30 | 40 | 30 | 70 | 30 | 20 | 30 | 0 | 20 | 0 | 0 | 10 | 0 | 20 | 20 |
| Velvetleaf | 40 | 40 | 50 | 30 | 0 | 0 | 50 | 30 | 30 | 40 | 30 | 20 | 60 | 80 | 40 | 60 | 50 | 20 | 20 | 30 | 20 | 30 | 0 | 0 | 0 | 30 | 50 |
| Wheat | 20 | 20 | 20 | 20 | 0 | 0 | 0 | 0 | 0 | 0 | 0 | 0 | 40 | 40 | 30 | 60 | 50 | 50 | 60 | 20 | 20 | 30 | 10 | 0 | 30 | 40 | 50 |
| | 500 g ai/ha | | 250 g ai/ha | | | | | | | 125 g ai/ha | | | | | | | | | | | | | | | | | |
| | Compounds | | Compounds | | | | | | | Compounds | | | | | | | | | | | | | | | | | |
| | 444 | 445 | 54 | 72 | 73 | 74 | 152 | 191 | 192 | 1 | 2 | 3 | 4 | 5 | 6 | 7 | 8 | 9 | 10 | 11 | 12 | 13 | 14 | 15 | 16 | 17 | 18 |
| Barnyardgrass | 0 | 40 | 0 | 0 | 10 | 0 | 40 | 10 | 10 | 0 | 30 | 10 | 10 | 10 | 0 | 0 | 0 | 0 | 0 | 0 | 0 | 10 | 10 | 0 | 10 | 0 | 10 |
| Blackgrass | 0 | 50 | 0 | 50 | 30 | 50 | 20 | 10 | 0 | 20 | 10 | 0 | 0 | 0 | 20 | 0 | 0 | 30 | 40 | 40 | 10 | 10 | 20 | 10 | 50 | 20 | 20 |
| Cocklebur | 10 | 80 | 10 | 0 | 20 | 0 | 90 | 10 | 60 | 60 | 50 | 30 | 30 | 20 | 0 | 0 | 0 | 50 | 20 | 10 | 10 | 10 | 10 | 0 | — | 10 | 10 |
| Corn | 10 | 40 | — | 0 | 30 | 0 | 30 | 10 | 10 | 0 | 20 | 20 | 20 | 20 | 0 | 0 | 0 | 0 | 0 | 0 | 0 | 0 | 0 | 0 | 10 | 10 | 0 |
| Crabgrass, Large | 10 | 40 | 0 | 0 | 10 | 10 | 20 | 20 | 20 | 0 | 20 | 0 | 20 | 20 | 20 | 0 | 0 | 0 | 10 | 0 | 0 | 20 | 10 | 10 | 10 | 0 | 0 |
| Foxtail, Giant | 10 | 30 | 0 | 0 | 30 | 10 | 20 | 20 | 20 | 20 | 30 | 20 | 20 | 20 | 10 | 0 | 0 | 0 | 20 | 20 | 0 | 30 | 20 | 20 | 10 | 10 | 10 |
| Lambsquarters | 20 | 80 | 40 | 10 | 10 | 10 | 80 | 80 | 80 | 60 | 80 | 60 | 80 | 60 | 30 | 0 | 0 | 70 | 80 | 60 | 20 | 50 | 50 | 20 | 60 | 60 | 10 |
| Morningglory | 20 | 80 | — | 0 | 10 | 10 | 60 | 90 | 100 | 0 | 30 | 20 | 40 | 0 | 0 | 0 | 0 | 0 | 20 | 0 | 0 | 10 | 0 | 0 | 0 | 0 | 10 |
| Oat, Wild | 0 | 60 | 0 | 30 | 40 | 40 | 20 | 20 | 20 | 40 | 20 | 20 | 20 | 20 | 10 | 0 | 0 | 40 | 20 | 20 | 10 | 10 | 10 | 10 | 30 | 30 | 20 |
| Pigweed | 20 | 60 | 10 | 0 | 20 | 20 | 90 | 70 | 60 | 50 | 60 | 30 | 40 | 30 | 20 | 0 | 0 | 70 | 80 | 20 | 0 | 30 | 30 | 20 | 40 | 30 | 10 |
| Surinam Grass | 10 | 30 | — | 0 | 10 | 10 | 40 | 10 | 10 | 0 | 20 | 10 | 10 | 20 | 10 | 0 | 0 | 0 | 20 | 10 | 0 | 10 | 10 | 10 | 10 | 10 | 10 |
| Velvetleaf | 0 | 50 | 0 | 0 | 50 | 30 | 50 | 20 | 50 | 40 | 20 | 10 | 20 | 20 | 0 | 0 | 0 | 20 | 70 | 10 | 0 | 20 | 40 | 10 | 40 | 10 | 20 |
| Wheat | 0 | 50 | 0 | 10 | 30 | 20 | 10 | 0 | 0 | 10 | 20 | 0 | 10 | 0 | 0 | 0 | 0 | 10 | 10 | 10 | 10 | 0 | 0 | 10 | 20 | 20 | 0 |
| | 125 g ai/ha | | | | | | | | | | | | | | | | | | | | | | | | | | |
| | Compounds | | | | | | | | | | | | | | | | | | | | | | | | | | |
| | 19 | 20 | 21 | 22 | 23 | 24 | 25 | 26 | 27 | 28 | 29 | 30 | 31 | 32 | 33 | 34 | 35 | 36 | 37 | 40 | 41 | 43 | 44 | 45 | 46 | 47 | 48 |
| Barnyardgrass | 0 | 0 | 0 | 0 | 0 | 0 | 0 | 0 | 0 | 10 | 0 | 10 | 10 | 0 | 20 | 20 | 10 | 20 | 20 | 10 | 10 | 20 | 20 | 20 | 10 | 20 | 40 |
| Blackgrass | 10 | 30 | 20 | 10 | 20 | 30 | 30 | 0 | 60 | 10 | 0 | 10 | 0 | 0 | 20 | 10 | 20 | 10 | 10 | 10 | 10 | 20 | 10 | 10 | 10 | 10 | 0 |
| Cocklebur | 0 | 0 | 0 | 0 | 0 | 10 | 0 | 0 | 0 | 30 | 50 | 70 | 80 | 10 | 80 | 40 | 50 | 30 | 60 | 10 | 10 | 10 | 40 | 20 | 30 | 10 | 0 |
| Corn | 0 | 0 | 0 | 0 | 0 | 0 | 0 | 0 | 30 | 0 | 10 | 10 | 30 | 10 | — | — | — | — | — | — | — | 20 | 20 | 20 | 20 | 10 | 0 |
| Crabgrass, Large | 10 | 10 | 10 | 0 | 0 | 0 | 0 | 0 | 70 | 10 | 20 | 10 | 20 | 0 | 20 | 20 | 20 | 20 | 10 | 10 | 20 | 90 | 20 | 30 | 30 | 10 | 10 |
| Foxtail, Giant | 0 | 10 | 10 | 0 | 0 | 0 | 0 | 0 | 30 | 20 | 10 | 10 | 20 | 0 | 30 | 30 | 20 | 20 | 20 | 0 | 10 | 30 | 20 | 20 | 30 | 30 | 20 |
| Lambsquarters | 10 | 30 | 10 | 0 | 0 | 10 | 0 | 0 | 90 | 80 | 70 | 80 | 90 | 70 | 90 | 80 | 80 | 80 | 80 | 40 | 60 | 90 | 80 | 80 | 80 | 80 | 60 |
| Morningglory | 10 | 10 | 0 | 10 | 0 | 10 | 0 | 0 | 20 | 0 | 0 | 10 | 30 | 0 | — | — | — | — | — | — | — | 80 | 30 | 40 | 0 | 30 | 0 |
| Oat, Wild | 10 | 30 | 10 | 10 | 0 | 20 | 30 | 0 | 50 | 20 | 10 | 20 | 20 | 10 | 10 | 10 | 0 | 20 | 10 | 0 | 10 | 10 | 20 | 0 | 0 | 10 | 10 |
| Pigweed | 10 | 10 | 30 | 10 | 10 | 20 | 0 | 0 | 90 | 90 | 100 | 30 | 60 | 40 | 80 | 90 | 80 | 80 | 60 | 20 | 50 | 100 | 90 | 90 | 90 | 90 | 50 |
| Surinam Grass | 10 | 10 | 10 | 0 | 0 | 0 | 0 | 0 | 40 | 20 | 20 | 20 | 30 | 10 | 10 | 20 | 20 | 10 | 10 | 10 | 10 | 40 | 20 | 20 | 30 | 20 | 30 |
| Velvetleaf | 0 | 20 | 10 | 0 | 0 | 10 | 0 | 0 | 30 | 30 | 60 | 20 | 30 | 10 | 50 | 40 | 30 | 20 | 20 | 10 | 10 | 80 | 50 | 60 | 60 | 60 | 10 |
| Wheat | 0 | 20 | 10 | 0 | 0 | 20 | 10 | 0 | 10 | 10 | 0 | 0 | 10 | 0 | 20 | 30 | 20 | 10 | 20 | 10 | 0 | 20 | 20 | 20 | 20 | 20 | 0 |
| | 125 g ai/ha | | | | | | | | | | | | | | | | | | | | | | | | | | |
| | Compounds | | | | | | | | | | | | | | | | | | | | | | | | | | |
| | 49 | 50 | 51 | 52 | 55 | 56 | 57 | 58 | 59 | 60 | 61 | 62 | 63 | 64 | 65 | 66 | 67 | 68 | 69 | 70 | 71 | 75 | 76 | 77 | 78 | 79 | 80 |
| Barnyardgrass | 30 | 50 | 60 | 0 | 10 | 20 | 10 | 30 | 0 | 10 | 20 | 20 | 10 | 10 | 10 | 0 | 10 | 0 | 0 | 20 | 0 | 10 | 10 | 0 | 30 | 30 | 80 |
| Blackgrass | 0 | 20 | 0 | 0 | — | 0 | 0 | 10 | 0 | 0 | 0 | 10 | 10 | 0 | 10 | 0 | 10 | 10 | 10 | 10 | 0 | 10 | 10 | 20 | 20 | 20 | 20 |
| Cocklebur | 20 | 90 | 70 | 20 | 10 | 80 | 30 | 80 | 10 | 40 | 60 | 70 | 60 | 30 | 60 | 20 | 0 | 10 | 0 | 50 | 0 | 10 | 40 | 0 | 80 | 80 | 70 |
| Corn | 20 | 30 | 0 | 10 | 20 | 20 | 20 | 10 | 10 | 10 | 20 | 10 | 20 | 10 | 20 | 10 | 10 | 0 | 0 | 20 | 0 | 10 | 10 | 10 | — | — | 20 |
| Crabgrass, Large | 10 | 10 | 20 | 0 | 10 | 10 | 10 | 20 | 10 | 10 | 20 | 10 | 10 | 10 | 10 | 0 | 10 | 0 | 0 | 20 | 0 | 10 | 10 | 0 | 20 | 20 | 100 |
| Foxtail, Giant | 10 | 30 | 0 | 10 | 0 | 20 | 20 | 20 | 0 | 10 | 20 | 10 | 10 | 10 | 10 | 0 | 10 | 0 | 0 | 20 | 0 | 20 | 20 | 0 | 30 | 30 | 50 |
| Lambsquarters | 50 | 80 | 80 | 0 | 70 | 80 | 70 | 90 | 80 | 80 | 90 | 80 | 80 | 70 | 70 | 60 | 40 | 0 | 0 | 80 | 0 | 40 | 30 | 50 | 70 | 70 | 70 |
| Morningglory | 0 | 70 | 0 | 0 | 10 | 0 | 10 | 20 | 20 | 10 | 20 | 20 | 20 | 0 | 10 | 20 | 0 | 0 | 0 | 20 | 0 | 20 | 10 | 0 | — | — | 100 |
| Oat, Wild | 10 | 20 | 0 | 0 | 10 | 20 | 30 | 20 | 10 | 20 | 20 | 10 | 20 | 20 | 10 | 10 | 10 | 10 | 10 | 20 | 0 | 10 | 30 | 40 | 10 | 20 | 30 |
| Pigweed | 30 | 80 | 80 | 0 | 10 | 60 | 70 | 60 | 70 | 70 | 20 | 80 | 40 | 20 | 40 | 20 | 0 | 10 | 10 | 30 | 0 | 50 | 100 | 70 | 80 | 70 | 100 |
| Surinam Grass | 10 | 30 | 10 | 0 | 10 | 20 | 10 | 10 | 10 | 10 | 20 | 50 | 10 | 10 | 20 | 10 | 10 | 0 | 0 | 20 | 0 | 10 | 20 | 0 | 20 | 30 | 90 |
| Velvetleaf | 30 | 40 | 60 | 10 | 20 | 40 | 20 | 50 | 20 | 20 | 30 | 60 | 40 | 10 | 10 | 10 | 10 | 0 | 0 | 20 | 0 | 20 | 40 | 20 | 70 | 70 | 90 |
| Wheat | 0 | 30 | 20 | 0 | 10 | 10 | 10 | 0 | 0 | 0 | 10 | 0 | 10 | 0 | 0 | 0 | 10 | 0 | 0 | 10 | 0 | 10 | 20 | 10 | 30 | 30 | 20 |
| | 125 g ai/ha | | | | | | | | | | | | | | | | | | | | | | | | | | |
| | Compounds | | | | | | | | | | | | | | | | | | | | | | | | | | |
| | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 104 | 105 | 106 | 107 | 108 |
| Barnyardgrass | 70 | 10 | 10 | 20 | 60 | 50 | 0 | 30 | 10 | 0 | 0 | 0 | 10 | 10 | 0 | 0 | 0 | 10 | 0 | 0 | 10 | 0 | 0 | 0 | 0 | 0 | 10 |
| Blackgrass | 0 | 0 | 0 | 0 | 10 | 10 | 0 | 0 | 0 | 0 | 0 | 0 | 10 | 0 | 0 | 0 | 0 | 0 | 20 | 10 | 20 | 0 | 0 | 0 | 0 | 0 | 0 |
| Cocklebur | 70 | 80 | 0 | 20 | 90 | 20 | 0 | 10 | 70 | 10 | 0 | 0 | 50 | 20 | 60 | 90 | 0 | 0 | 0 | 0 | 20 | 30 | 0 | 30 | 20 | 0 | 70 |
| Corn | 10 | 20 | 10 | 10 | 20 | 20 | 0 | 10 | 10 | 10 | 10 | 10 | 10 | 20 | 10 | 10 | 0 | 10 | 10 | 0 | 10 | 20 | 0 | 0 | 10 | 0 | 10 |
| Crabgrass, Large | 10 | 10 | 30 | 20 | 50 | 60 | 0 | 30 | 10 | 10 | 0 | 0 | 10 | 10 | 0 | 40 | 0 | 20 | 10 | 0 | 10 | 10 | 0 | 0 | 0 | 0 | 20 |
| Foxtail, Giant | 20 | 10 | 0 | 10 | 20 | 40 | 0 | 30 | 20 | 10 | 0 | 0 | 10 | 10 | 10 | 30 | 0 | 10 | 10 | 0 | 20 | 10 | 0 | 0 | 0 | 0 | 10 |
| Lambsquarters | 0 | 20 | 0 | 0 | 50 | 80 | 0 | 70 | 30 | 50 | 20 | 20 | 60 | 50 | 100 | 100 | 50 | 20 | 70 | 0 | 80 | 10 | 0 | 50 | 50 | 10 | 90 |
| Morningglory | 0 | 10 | 0 | 30 | 50 | 60 | 0 | 0 | 20 | 10 | 0 | 0 | 10 | 10 | 0 | 0 | 0 | 0 | 0 | 0 | 10 | 10 | 0 | 0 | 10 | 0 | 30 |
| Oat, Wild | 20 | 10 | 10 | 20 | 20 | 30 | 0 | 0 | 20 | 10 | 0 | 0 | 10 | 10 | 10 | 20 | 0 | 0 | 20 | 20 | 30 | 20 | 0 | 0 | 10 | 0 | 20 |
| Pigweed | 50 | 80 | 70 | 80 | 100 | 100 | 0 | 50 | 20 | 30 | 10 | 0 | 10 | 10 | 90 | 70 | 0 | 10 | 20 | 0 | 80 | 80 | 10 | 0 | 10 | 0 | 80 |
| Surinam Grass | 40 | 30 | 20 | 50 | 60 | 70 | 0 | 0 | 10 | 10 | 10 | 0 | 10 | 10 | 20 | 20 | 0 | 10 | 10 | 0 | 10 | 70 | 0 | 0 | 10 | 10 | 10 |
| Velvetleaf | 80 | 80 | 50 | 60 | 60 | 60 | 0 | 30 | 30 | 10 | 0 | 0 | 10 | 10 | 50 | 70 | 0 | 10 | 20 | 0 | 20 | 0 | 0 | 0 | 0 | 0 | 50 |
| Wheat | 20 | 20 | 20 | 20 | 50 | 30 | 0 | 0 | 20 | 10 | 0 | 0 | 10 | 0 | 10 | 10 | 0 | 0 | 20 | 10 | 10 | 0 | 0 | 0 | 0 | 0 | 10 |
| | 125 g ai/ha | | | | | | | | | | | | | | | | | | | | | | | | | | |
| | Compounds | | | | | | | | | | | | | | | | | | | | | | | | | | |
| | 109 | 110 | 112 | 113 | 114 | 115 | 117 | 118 | 119 | 120 | 121 | 122 | 123 | 124 | 125 | 126 | 127 | 128 | 130 | 131 | 132 | 133 | 134 | 135 | 136 | 137 | 138 |
| Barnyardgrass | 10 | 10 | 50 | 30 | 20 | 10 | 20 | 10 | 0 | 10 | 0 | 0 | 0 | 0 | 0 | 0 | 10 | 20 | 50 | 10 | 50 | 0 | 20 | 20 | 50 | 30 | 10 |
| Blackgrass | 0 | 0 | 20 | 10 | 10 | 10 | 0 | 0 | 0 | 0 | 0 | 0 | 0 | 0 | 0 | 0 | 10 | 10 | 20 | 10 | 10 | 0 | 0 | 0 | 30 | 10 | 0 |
| Cocklebur | 30 | 10 | 60 | 100 | 80 | 80 | 30 | 70 | 0 | 30 | 10 | 70 | 0 | 0 | 0 | 50 | 10 | 90 | 100 | 70 | 90 | 10 | 80 | 40 | 100 | 50 | 40 |
| Corn | 10 | 10 | 40 | 30 | — | — | — | 20 | 10 | — | 0 | 10 | 0 | 0 | 0 | 0 | — | — | 20 | 20 | 20 | 10 | 20 | 20 | 10 | 10 | 20 |
| Crabgrass, Large | 10 | 10 | 20 | 30 | 30 | 10 | 10 | 20 | 20 | 10 | 10 | 10 | 0 | 10 | 0 | 40 | 20 | 20 | 70 | 20 | 30 | 10 | 30 | 20 | 10 | 10 | 20 |
| Foxtail, Giant | 20 | 10 | 20 | 30 | 30 | 20 | 10 | 10 | 10 | 10 | 0 | 10 | 0 | 0 | 0 | 20 | 20 | 20 | 20 | 20 | 30 | 0 | 30 | 30 | 10 | 10 | 20 |
| Lambsquarters | 10 | 40 | 90 | 90 | 80 | 80 | 60 | 0 | 0 | 0 | 80 | 0 | 0 | 30 | 0 | 0 | 70 | 0 | 90 | 80 | 100 | 60 | 80 | 70 | 100 | 80 | 80 |
| Morningglory | 0 | 20 | 20 | 20 | — | — | — | 20 | 0 | — | 0 | 0 | 0 | 0 | 0 | 0 | — | — | 100 | 20 | 60 | 10 | 20 | 30 | 20 | 0 | 20 |
| Oat, Wild | 20 | 0 | 40 | 40 | 10 | 10 | 10 | 20 | 10 | 10 | 10 | 10 | 0 | 0 | 0 | 30 | 10 | 10 | 40 | 20 | 20 | 20 | 30 | 20 | 50 | 30 | 20 |
| Pigweed | 20 | 20 | 90 | 90 | 90 | 80 | 50 | 60 | 0 | 10 | 30 | 0 | 0 | 20 | 0 | 40 | 30 | 20 | 100 | 30 | 70 | 20 | 80 | 80 | 60 | 90 | 60 |
| Surinam Grass | 10 | 20 | 20 | 40 | 10 | 60 | 30 | 40 | 10 | 20 | 10 | 10 | 0 | 10 | 0 | 20 | 10 | 10 | 50 | 20 | 40 | 0 | 20 | 30 | 20 | 20 | 20 |
| Velvetleaf | 10 | 30 | 30 | 20 | 50 | 60 | 20 | 20 | 0 | 10 | 10 | 10 | 0 | 0 | 0 | 0 | 0 | 50 | 70 | 10 | 60 | 10 | 30 | 20 | 60 | 40 | 20 |
| Wheat | 10 | 10 | 20 | 10 | 20 | 30 | 10 | 10 | 0 | 10 | 0 | 0 | 0 | 0 | 0 | 0 | 0 | 0 | 20 | 10 | 10 | 0 | 10 | 20 | 20 | 10 | 20 |
| | 125 g ai/ha | | | | | | | | | | | | | | | | | | | | | | | | | | |
| | Compounds | | | | | | | | | | | | | | | | | | | | | | | | | | |
| | 139 | 140 | 141 | 142 | 143 | 144 | 145 | 146 | 147 | 148 | 149 | 150 | 151 | 153 | 154 | 155 | 156 | 157 | 158 | 159 | 160 | 161 | 162 | 163 | 164 | 165 | 166 |
| Barnyardgrass | 10 | 0 | 0 | 0 | 0 | 70 | 0 | 20 | 20 | 0 | 0 | 10 | 0 | 30 | 20 | 10 | 10 | 0 | 0 | 0 | 0 | 10 | 0 | 0 | 0 | 0 | 0 |
| Blackgrass | 0 | 0 | 20 | 40 | 0 | 20 | 10 | 10 | 10 | 0 | 10 | 0 | 10 | 10 | 10 | 40 | 30 | 10 | 20 | — | — | 40 | 20 | 10 | 0 | 0 | 0 |
| Cocklebur | 20 | 0 | 0 | 40 | 0 | 30 | 0 | 20 | 0 | 20 | 20 | 60 | 0 | 50 | 60 | 60 | 40 | 10 | 20 | 80 | 20 | 20 | 10 | 10 | 0 | 0 | 0 |
| Corn | 10 | 0 | 0 | 0 | 0 | 20 | 10 | 10 | 10 | 10 | 10 | 10 | 10 | 10 | 10 | 10 | 0 | 0 | 0 | 0 | 0 | 0 | 0 | 0 | 0 | 0 | 0 |
| Crabgrass, Large | 10 | 0 | 10 | 0 | 0 | 30 | 10 | 10 | 20 | 10 | 10 | 10 | 0 | 10 | 10 | 20 | 10 | 0 | 30 | 0 | 0 | 0 | 20 | 20 | 0 | 0 | 0 |
| Foxtail, Giant | 10 | 0 | 20 | 30 | 0 | 40 | 30 | 20 | 30 | 10 | 10 | 0 | 10 | 10 | 10 | 20 | 20 | 30 | 0 | 40 | 30 | 20 | 20 | 0 | 0 | 0 | 0 |
| Lambsquarters | 80 | 20 | 60 | 60 | 0 | 90 | 60 | 80 | 80 | 10 | 20 | 50 | 0 | 80 | 80 | 70 | 60 | 20 | 80 | 90 | 70 | 70 | 80 | 50 | 0 | 0 | 0 |
| Morningglory | 10 | 0 | 20 | 0 | 0 | 50 | 0 | 0 | 0 | 10 | 20 | 20 | 10 | 10 | 60 | 30 | 20 | 0 | 0 | 10 | 0 | 20 | 0 | 0 | 0 | 0 | 0 |
| Oat, Wild | 10 | 0 | 10 | 20 | 0 | 20 | 10 | 10 | 0 | 10 | 10 | 20 | 10 | 20 | 10 | 40 | 40 | 10 | 30 | — | 10 | 30 | 20 | 10 | 0 | 0 | 0 |
| Pigweed | 60 | 0 | 30 | 50 | 0 | 100 | 90 | 80 | 80 | 10 | 50 | 70 | 10 | 20 | 20 | 60 | 30 | 40 | 50 | 80 | 40 | 20 | 40 | 60 | 0 | 0 | 0 |
| Surinam Grass | 10 | 0 | 10 | 10 | 10 | 60 | 20 | 10 | 20 | 10 | 10 | 10 | 10 | 30 | 30 | 20 | 10 | 20 | 0 | 0 | 10 | 20 | 0 | 0 | 0 | 0 | 0 |
| Velvetleaf | 20 | 0 | 20 | 10 | 0 | 80 | 50 | 50 | 60 | 10 | 10 | 20 | 10 | 50 | 20 | 20 | 60 | 50 | 50 | 20 | 70 | 20 | 60 | 40 | 0 | 0 | 0 |
| Wheat | 10 | 0 | 20 | 10 | 0 | 20 | 0 | 0 | 0 | 10 | 10 | 10 | 10 | 10 | 10 | 20 | 20 | 10 | 10 | — | — | 10 | 10 | 0 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 167 | 168 | 169 | 170 | 171 | 179 | 180 | 181 | 182 | 183 | 184 | 185 | 186 | 187 | 188 | 189 | 190 | 193 | 194 | 195 | 196 | 197 | 198 | 199 | 200 | 201 | 202 |
| Barnyardgrass | 0 | 50 | 0 | 0 | 0 | 0 | 0 | 0 | 30 | 0 | 0 | 30 | 10 | 10 | 10 | 0 | 0 | 0 | 0 | 40 | 0 | 0 | 0 | 0 | 0 | 0 | 0 |
| Blackgrass | 0 | 10 | 0 | 0 | 0 | 0 | 0 | 0 | 0 | 0 | 0 | 0 | 10 | 0 | 10 | 0 | 0 | 0 | 0 | 0 | 0 | 0 | 0 | 0 | 0 | 0 | 0 |
| Cocklebur | 0 | 70 | 0 | 0 | 0 | 0 | 0 | 20 | 50 | 50 | 30 | 20 | 60 | 30 | 20 | 0 | 0 | 20 | 10 | 30 | 0 | 0 | 60 | 10 | 10 | 10 | 30 |
| Corn | 0 | 50 | 10 | 0 | 0 | 0 | 0 | 20 | 30 | 20 | 20 | 20 | 20 | 0 | 10 | 10 | 10 | 10 | 0 | 20 | 0 | 10 | 20 | 10 | 10 | 10 | 10 |
| Crabgrass, Large | 0 | 20 | 10 | 10 | 0 | 0 | 0 | 10 | 20 | 20 | 20 | 20 | 10 | 10 | 10 | 10 | 10 | 10 | 10 | 20 | 30 | 0 | 10 | 10 | 0 | 0 | 0 |
| Foxtail, Giant | 0 | 20 | 0 | 0 | 10 | 0 | 0 | 20 | 0 | 20 | 30 | 20 | 10 | 10 | 10 | 0 | 10 | 10 | 0 | 20 | 0 | 0 | 20 | 10 | 10 | 0 | 10 |
| Lambsquarters | 0 | 70 | 40 | 60 | 10 | 0 | 0 | 70 | 60 | 70 | 60 | 60 | 10 | 0 | 60 | 50 | 30 | 90 | — | 0 | 0 | 0 | 70 | 60 | 60 | 40 | 50 |
| Morningglory | 0 | 70 | 20 | 30 | 0 | 0 | 0 | 20 | 50 | 60 | 0 | 40 | 20 | 20 | 20 | 80 | 50 | 30 | 10 | 0 | 0 | 0 | 60 | 40 | 0 | 0 | 20 |
| Oat, Wild | 0 | 20 | 10 | 10 | 0 | 0 | 0 | 20 | 20 | 10 | 20 | 10 | 10 | 10 | 20 | 10 | 10 | 0 | 0 | 10 | 0 | 10 | 20 | 10 | 0 | 10 | 10 |
| Pigweed | 0 | 70 | 20 | 30 | 10 | 0 | 0 | 40 | 50 | 70 | 20 | 60 | 60 | 20 | 50 | 30 | 30 | 20 | 20 | 70 | 10 | 0 | 50 | 10 | 10 | 10 | 30 |
| Surinam Grass | 0 | 20 | 0 | 10 | 10 | 0 | 0 | 20 | 10 | 0 | 10 | 20 | 10 | 10 | 10 | 10 | 10 | 10 | 10 | 10 | 0 | 10 | 20 | 10 | 10 | 0 | 10 |
| Velvetleaf | 0 | 20 | 0 | 10 | 0 | 0 | 0 | 10 | 0 | 40 | 30 | 30 | 60 | 0 | 10 | 20 | 20 | 50 | 10 | 20 | 0 | 0 | 20 | 20 | 10 | 0 | 10 |
| Wheat | 0 | 20 | 0 | 0 | 0 | 0 | 0 | 0 | 0 | 0 | 0 | 0 | 10 | 0 | 0 | 0 | 0 | 10 | 0 | 10 | 0 | 0 | 0 | 0 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 203 | 204 | 205 | 206 | 207 | 209 | 210 | 211 | 212 | 213 | 214 | 215 | 216 | 217 | 218 | 219 | 220 | 221 | 222 | 223 | 224 | 225 | 226 | 227 | 228 | 229 | 230 |
| Barnyardgrass | 0 | 10 | 10 | 0 | 20 | 10 | 20 | 10 | 10 | 0 | 0 | 0 | 0 | 0 | 0 | 0 | 0 | 0 | 10 | 10 | 10 | 10 | 10 | — | 30 | 20 | 0 |
| Blackgrass | 0 | 20 | 0 | 0 | 0 | 0 | 10 | 10 | 10 | 20 | 0 | 0 | 0 | 0 | 0 | 0 | 0 | 0 | 0 | 0 | 0 | 20 | 20 | 20 | 30 | 30 | 30 |
| Cocklebur | 0 | 90 | 40 | 20 | 40 | 30 | 60 | 30 | 80 | 0 | 0 | 20 | 0 | 0 | 0 | 0 | 0 | 0 | 20 | 10 | 10 | 20 | 10 | 20 | 30 | 20 | 0 |
| Corn | 0 | 20 | 20 | 20 | 20 | 20 | 20 | 10 | 20 | 0 | 0 | 0 | 0 | 0 | 0 | 0 | 10 | 10 | 0 | 10 | 10 | 10 | 10 | 30 | 10 | 10 | 10 |
| Crabgrass, Large | 0 | 10 | 10 | 10 | 20 | 20 | 20 | 10 | 10 | 0 | 0 | 0 | 0 | 0 | 0 | 0 | 10 | 0 | 10 | 10 | 10 | 10 | 20 | 30 | 20 | 10 | 10 |
| Foxtail, Giant | 0 | 10 | 20 | 20 | 20 | 20 | 20 | 10 | 10 | 0 | 0 | 0 | 0 | 0 | 0 | 0 | 0 | 10 | 10 | 10 | 20 | 10 | 20 | 10 | 30 | 30 | 10 |
| Lambsquarters | 10 | 30 | 60 | 30 | 50 | 40 | 70 | 60 | 90 | 0 | 0 | 0 | 0 | 0 | 0 | 0 | 10 | 0 | 50 | 60 | 60 | 50 | 40 | 50 | 70 | 70 | 50 |
| Morningglory | 0 | 20 | 30 | 20 | 10 | 20 | 30 | 10 | 10 | 10 | 0 | 0 | 0 | 0 | 0 | 0 | 0 | 0 | 40 | 40 | 30 | 10 | 30 | 50 | 70 | 10 | 20 |
| Oat, Wild | 0 | 10 | 20 | 20 | 20 | 20 | 20 | 10 | 20 | 10 | 0 | 10 | 0 | 0 | 0 | 0 | 0 | 0 | 0 | 0 | 0 | 0 | 30 | 0 | 40 | 30 | 20 |
| Pigweed | 0 | 50 | 40 | 30 | 30 | 30 | 40 | 10 | 60 | 0 | 0 | 20 | 0 | 0 | 0 | 10 | 10 | 10 | 40 | 60 | 40 | 40 | 30 | 20 | 50 | 60 | 40 |
| Surinam Grass | 10 | 20 | 20 | 10 | 20 | 20 | 20 | 10 | 30 | 10 | 0 | 0 | 0 | 0 | 0 | 0 | 0 | 0 | 10 | 10 | 10 | 10 | 10 | 10 | 20 | 10 | 10 |
| Velvetleaf | 0 | 60 | 20 | 20 | 20 | 20 | 30 | 30 | 50 | 0 | 0 | 40 | 0 | 0 | 0 | 0 | 0 | 0 | 50 | 20 | 20 | 30 | 20 | 20 | 60 | 60 | 20 |
| Wheat | 0 | 10 | 10 | 20 | 20 | 10 | 20 | 10 | 10 | 0 | 0 | 0 | 0 | 0 | 0 | 0 | 0 | 0 | 0 | 0 | 0 | 10 | 20 | 0 | 30 | 30 | 10 |
| | 125 g ai/ha | | | | | | | | | | | | | | | | | | | | | | | | | | |
| | Compounds | | | | | | | | | | | | | | | | | | | | | | | | | | |
| | 231 | 232 | 233 | 234 | 235 | 236 | 237 | 238 | 239 | 240 | 241 | 242 | 243 | 244 | 245 | 246 | 247 | 248 | 249 | 250 | 252 | 253 | 254 | 256 | 257 | 258 | 259 |
| Barnyardgrass | 10 | — | — | — | — | — | — | 0 | 0 | 0 | 0 | 50 | 10 | 10 | 40 | 30 | 0 | 10 | 10 | 20 | 0 | 0 | 0 | 0 | 0 | 0 | 0 |
| Blackgrass | 20 | 0 | 0 | 0 | 0 | 0 | 0 | 0 | 0 | 0 | 0 | 10 | 0 | 10 | 0 | 0 | 0 | 20 | 20 | 10 | 10 | 20 | 0 | 0 | 0 | 10 | 10 |
| Cocklebur | 10 | 10 | 20 | 10 | 0 | 20 | 10 | 0 | 0 | 0 | 20 | 40 | 10 | 30 | 40 | 20 | 0 | 30 | 30 | 30 | 20 | 10 | 20 | 0 | 0 | 20 | 0 |
| Corn | 10 | 10 | 10 | 0 | 20 | 20 | 20 | 0 | 0 | 0 | 0 | 10 | 10 | 10 | 10 | 0 | 0 | 20 | 10 | 20 | 10 | 0 | 0 | 0 | 0 | 0 | 10 |
| Crabgrass, Large | 20 | 10 | 10 | 10 | 10 | 0 | 10 | 0 | 0 | 0 | 0 | 30 | 10 | 10 | 10 | 10 | 10 | 10 | 10 | 20 | 0 | 0 | 10 | 0 | 0 | 0 | 0 |
| Foxtail, Giant | 20 | 10 | 0 | 0 | 10 | 10 | 10 | 0 | 0 | 0 | 0 | 20 | 10 | 20 | 30 | 30 | 10 | 20 | 20 | 20 | 20 | 0 | 0 | 0 | 0 | 0 | 0 |
| Lambsquarters | 60 | 50 | 50 | 20 | 40 | 60 | 60 | 0 | 0 | 20 | 30 | 90 | 70 | 40 | 80 | 50 | 60 | 60 | 10 | 0 | 30 | 20 | 10 | 30 | 20 | 30 | 10 |
| Morningglory | 20 | 30 | 50 | 20 | — | 10 | 20 | 0 | 0 | 20 | 20 | 20 | 0 | 10 | 10 | 0 | 20 | 20 | 20 | 20 | 20 | 0 | 20 | 0 | 0 | 0 | 0 |
| Oat, Wild | 20 | 0 | 0 | 20 | 10 | 20 | 0 | 0 | 0 | 0 | 0 | 10 | 0 | 30 | 20 | 10 | 0 | 30 | 30 | 20 | 20 | 20 | 20 | 10 | 10 | 20 | 10 |
| Pigweed | 50 | — | 60 | 30 | 30 | 60 | 30 | 0 | 20 | 0 | 30 | 60 | 20 | 50 | 60 | 50 | 60 | 60 | 20 | 30 | 20 | 20 | 10 | 30 | 60 | 50 | 30 |
| Surinam Grass | 10 | 10 | 10 | 0 | 10 | 0 | 20 | 0 | 0 | 0 | 10 | 30 | 10 | 20 | 30 | 20 | 10 | 10 | 10 | 10 | 10 | 0 | 10 | 0 | 0 | 0 | 10 |
| Velvetleaf | 30 | 30 | 30 | 20 | 20 | 20 | 30 | 0 | 0 | 0 | 10 | 50 | 50 | 40 | 70 | 30 | 30 | 20 | 10 | 20 | 30 | 10 | 10 | 10 | 0 | 0 | 0 |
| Wheat | 20 | 0 | 0 | 0 | 0 | 10 | 10 | 0 | 0 | 0 | 0 | 10 | 0 | 10 | 10 | 0 | 0 | 30 | 30 | 20 | 20 | 20 | 10 | 10 | 0 | 20 | 10 |
| | 125 g ai/ha | | | | | | | | | | | | | | | | | | | | | | | | | | |
| | Compounds | | | | | | | | | | | | | | | | | | | | | | | | | | |
| | 260 | 261 | 262 | 263 | 264 | 265 | 266 | 267 | 268 | 269 | 270 | 271 | 272 | 273 | 274 | 275 | 276 | 277 | 278 | 279 | 280 | 281 | 282 | 283 | 284 | 285 | 286 |
| Barnyardgrass | 0 | 0 | 0 | 0 | 0 | 0 | 0 | 40 | 0 | 10 | 0 | 0 | 20 | 20 | 0 | 0 | 0 | 0 | 0 | 0 | 0 | 0 | 0 | 0 | 0 | 0 | 0 |
| Blackgrass | 10 | 0 | 0 | 10 | 0 | 0 | 20 | 20 | 30 | 20 | 0 | 10 | 60 | 50 | 30 | 40 | 30 | 10 | 20 | 10 | 20 | 20 | 20 | 20 | 20 | 30 | 10 |
| Cocklebur | 10 | 0 | 0 | 0 | 0 | 0 | 0 | 30 | 20 | 20 | 0 | 0 | 40 | 20 | 0 | 0 | 10 | 0 | 10 | 0 | 0 | 20 | 10 | 0 | 0 | 10 | 10 |
| Corn | 0 | 10 | 0 | 0 | 0 | 0 | 0 | 20 | 20 | 10 | 0 | 0 | 30 | 10 | 0 | 0 | 0 | 0 | 10 | 10 | 0 | 0 | 10 | 0 | 0 | 10 | 0 |
| Crabgrass, Large | 0 | 0 | 0 | 10 | 0 | 0 | 0 | 20 | 0 | 10 | 0 | 0 | 10 | 30 | 0 | 0 | 10 | 0 | 0 | 0 | 0 | 10 | 10 | 0 | 0 | 0 | 0 |
| Foxtail, Giant | 0 | 0 | 10 | 0 | 0 | 0 | 0 | 40 | 10 | 30 | 0 | 0 | 10 | 10 | 20 | 0 | 10 | 0 | 0 | 0 | 0 | 10 | 20 | 0 | 0 | 30 | 20 |
| Lambsquarters | 10 | 10 | 50 | 70 | 0 | 0 | 60 | 30 | 60 | 50 | 0 | 10 | 100 | 60 | 40 | 50 | 20 | 20 | 20 | 10 | 10 | 20 | 10 | 0 | 50 | 40 | 10 |
| Morningglory | 0 | 0 | 0 | 0 | 0 | 0 | 30 | 30 | 10 | 20 | 0 | 0 | 90 | 20 | 0 | 0 | 10 | 0 | 0 | 0 | 0 | 0 | 10 | 0 | 0 | 20 | 0 |
| Oat, Wild | 20 | 0 | 0 | 10 | 0 | 0 | 10 | 30 | 30 | 20 | 0 | 20 | 60 | 30 | 20 | 30 | 30 | 10 | 20 | 20 | 10 | 10 | 0 | 0 | 10 | 40 | 30 |
| Pigweed | 20 | 20 | 70 | 60 | 0 | 0 | 20 | 60 | 20 | 60 | 0 | 50 | 100 | 80 | 40 | 60 | 40 | 0 | 10 | 30 | 10 | 20 | 20 | 0 | 60 | 70 | 60 |
| Surinam Grass | 0 | 0 | 0 | 10 | 0 | 0 | 10 | 30 | 10 | 10 | 0 | 0 | 10 | 20 | 20 | 10 | 10 | 0 | 10 | 10 | 0 | 10 | 10 | 0 | 0 | 10 | 10 |
| Velvetleaf | 0 | 0 | 20 | 20 | 0 | 0 | 0 | 50 | 20 | 10 | 0 | 0 | 50 | 40 | 10 | 0 | 10 | 20 | 10 | 10 | 10 | 0 | 10 | 0 | 0 | 10 | 10 |
| Wheat | 20 | 0 | 0 | 10 | 0 | 0 | 20 | 20 | 30 | 20 | 0 | 30 | 20 | 30 | 20 | 20 | 20 | 20 | 30 | 30 | 20 | 30 | 30 | 20 | 20 | 30 | 20 |
| | 125 g ai/ha | | | | | | | | | | | | | | | | | | | | | | | | | | |
| | Compounds | | | | | | | | | | | | | | | | | | | | | | | | | | |
| | 287 | 288 | 289 | 290 | 291 | 292 | 293 | 294 | 295 | 296 | 297 | 298 | 299 | 300 | 301 | 302 | 303 | 304 | 305 | 306 | 307 | 308 | 309 | 310 | 311 | 312 | 313 |
| Barnyardgrass | 0 | 0 | 0 | 0 | 0 | 0 | 0 | 20 | 30 | 30 | 10 | 10 | 10 | 30 | 20 | 10 | 40 | 20 | 0 | 10 | 0 | 0 | 20 | 0 | 0 | 0 | 30 |
| Blackgrass | 10 | 20 | 10 | 20 | 0 | 10 | 30 | 20 | 30 | 30 | 10 | 20 | 20 | 10 | 10 | 10 | 20 | 10 | 20 | 10 | 10 | 10 | 10 | 10 | 0 | 0 | 0 |
| Cocklebur | 10 | 0 | 0 | 0 | 0 | 0 | 20 | 30 | 40 | 50 | 0 | 20 | 20 | 10 | 10 | 10 | 10 | 0 | 10 | 30 | 20 | 0 | 40 | 0 | 0 | 0 | 0 |
| Corn | 0 | 0 | 0 | 0 | 0 | 10 | 10 | 10 | 10 | 30 | 10 | 0 | 0 | 30 | 50 | 10 | 70 | 40 | 70 | 30 | — | 0 | 20 | 0 | 0 | 0 | 10 |
| Crabgrass, Large | 0 | 0 | 0 | 0 | 0 | 0 | 0 | 10 | 30 | 30 | 20 | 20 | 10 | 10 | 20 | 0 | 10 | 10 | 0 | 10 | 10 | 0 | 0 | 10 | 0 | 0 | 10 |
| Foxtail, Giant | 20 | 0 | 20 | 20 | 10 | 0 | 20 | 30 | 60 | 60 | 20 | 10 | 10 | 30 | 10 | 0 | 50 | 30 | 20 | 10 | 20 | 10 | 20 | 10 | 0 | 0 | 20 |
| Lambsquarters | 10 | 0 | 0 | 10 | 20 | 30 | 50 | 70 | 40 | 50 | 10 | 40 | 40 | 70 | 70 | 60 | 80 | 80 | 50 | 70 | 60 | 50 | 40 | 60 | 0 | 10 | 60 |
| Morningglory | 0 | 0 | 0 | 0 | 0 | 0 | 30 | 10 | 50 | 50 | 0 | 60 | 70 | 90 | 60 | 30 | 80 | 100 | 0 | — | 0 | 0 | 0 | 0 | 0 | 0 | 0 |
| Oat, Wild | 20 | 20 | 20 | 20 | 10 | 10 | 30 | 40 | 40 | 30 | 0 | 30 | 20 | 30 | 20 | 0 | 30 | 30 | 10 | 20 | 0 | 10 | 20 | 30 | 0 | 0 | 20 |
| Pigweed | 20 | 10 | 10 | 10 | 0 | 20 | 10 | 30 | 100 | 80 | 0 | 80 | 90 | 80 | 80 | 80 | 70 | 90 | 0 | 70 | 50 | 60 | 50 | 50 | 0 | 0 | 60 |
| Surinam Grass | 0 | 0 | 0 | 0 | 0 | 0 | 10 | 20 | 50 | 40 | 20 | 20 | 20 | 10 | 10 | 0 | 10 | 0 | 0 | 10 | 10 | 10 | 10 | 10 | 0 | 0 | 10 |
| Velvetleaf | 0 | 10 | 0 | 0 | 0 | 10 | 10 | 40 | 70 | 90 | 0 | 60 | 60 | 40 | 30 | 10 | 30 | 10 | 20 | 40 | 10 | 10 | 20 | 20 | 0 | 0 | 60 |
| Wheat | 10 | 20 | 20 | 20 | 0 | 0 | 20 | 30 | 20 | 20 | 0 | 20 | 20 | 10 | 10 | 0 | 10 | 10 | 0 | 10 | 0 | 10 | 20 | 10 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 314 | 315 | 316 | 317 | 318 | 319 | 329 | 330 | 331 | 332 | 333 | 334 | 335 | 336 | 338 | 340 | 341 | 342 | 343 | 344 | 345 | 346 | 347 | 348 | 349 | 350 | 351 |
| Barnyardgrass | 10 | 10 | 0 | 0 | 40 | 0 | 0 | 0 | 10 | 20 | 10 | 0 | 0 | 10 | 20 | 10 | 10 | 10 | 10 | 0 | 0 | 20 | 0 | 0 | 0 | 0 | 0 |
| Blackgrass | 10 | 0 | 0 | 0 | 10 | 0 | 20 | 0 | 10 | 40 | 30 | 30 | 10 | 10 | 20 | 20 | 10 | 20 | 0 | 0 | 0 | 40 | 30 | 20 | 20 | 0 | 0 |
| Cocklebur | 10 | 20 | 0 | 0 | 10 | 20 | 0 | 0 | 10 | 30 | 20 | 20 | 10 | 0 | 10 | 0 | 10 | 10 | 0 | 0 | 0 | 30 | 0 | 0 | 0 | 0 | 0 |
| Corn | 10 | 10 | 10 | 0 | 20 | 20 | 0 | 0 | 0 | 0 | 10 | 0 | 10 | 10 | 10 | 0 | 0 | 10 | 10 | 10 | 0 | 0 | 0 | 0 | 0 | 0 | 0 |
| Crabgrass, Large | 10 | 0 | 10 | 0 | 10 | 20 | 0 | 0 | 10 | 50 | 40 | 10 | 10 | 20 | 20 | 10 | 10 | 10 | 20 | 0 | 0 | 0 | 0 | 0 | 20 | 0 | 0 |
| Foxtail, Giant | 20 | 0 | 0 | 0 | 10 | 20 | 10 | 0 | 0 | 30 | 30 | 10 | 20 | 20 | 20 | 20 | 30 | 20 | 30 | 10 | 0 | 20 | 0 | 0 | 0 | 0 | 0 |
| Lambsquarters | 30 | 50 | 40 | 0 | 80 | 50 | 30 | 0 | 60 | 80 | 90 | 70 | 10 | 60 | 10 | 30 | 30 | 60 | 40 | 20 | 20 | 30 | 10 | 0 | 0 | 0 | 0 |
| Morningglory | 20 | 0 | 10 | 0 | 0 | 10 | 0 | 10 | 0 | 40 | 30 | 50 | 10 | 40 | 60 | 10 | 20 | 30 | 30 | 10 | 10 | 10 | 0 | 0 | 0 | 0 | 0 |
| Oat, Wild | 10 | 0 | 0 | 0 | 0 | 20 | 20 | 0 | 10 | 40 | 40 | 20 | 20 | 10 | 0 | 20 | 0 | 20 | 10 | 0 | 0 | 40 | 0 | 20 | 10 | 0 | 0 |
| Pigweed | 60 | 10 | 10 | 0 | 70 | 30 | 50 | 0 | 10 | 100 | 90 | 40 | 20 | 60 | 60 | 50 | 30 | 40 | 40 | 20 | 20 | 70 | 0 | 0 | 0 | 0 | 0 |
| Surinam Grass | 20 | 20 | 10 | 0 | 10 | 10 | 10 | 0 | 10 | 20 | 20 | 20 | 10 | 20 | 20 | 10 | 10 | 10 | 10 | 0 | 0 | 10 | 0 | 0 | 0 | 0 | 0 |
| Velvetleaf | 20 | 20 | 0 | 0 | 40 | 10 | 10 | 0 | 10 | 60 | 50 | 50 | 30 | 20 | 20 | 20 | 20 | 20 | 20 | 20 | 10 | 10 | 0 | 0 | 0 | 0 | 0 |
| Wheat | 0 | 0 | 0 | 0 | 0 | 10 | 20 | 0 | 20 | 20 | 20 | 10 | 10 | 0 | 0 | 0 | 10 | 0 | 0 | 0 | 0 | 20 | 0 | 20 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 352 | 353 | 354 | 355 | 356 | 357 | 358 | 359 | 360 | 361 | 362 | 363 | 364 | 365 | 366 | 368 | 369 | 370 | 371 | 372 | 373 | 374 | 375 | 376 | 377 | 378 | 379 |
| Barnyardgrass | 0 | 10 | 0 | 0 | 0 | 0 | 10 | 10 | 0 | 0 | 0 | 0 | 10 | 0 | 10 | 0 | 10 | 0 | 0 | 10 | 10 | 10 | 30 | 10 | 0 | 0 | 0 |
| Blackgrass | 10 | 10 | 10 | 0 | 0 | 0 | 10 | 20 | 30 | 10 | 10 | 20 | 30 | 40 | 40 | 0 | 0 | 0 | 0 | 10 | 0 | 10 | 30 | 10 | 0 | 0 | 0 |
| Cocklebur | 0 | 10 | 10 | 0 | — | 10 | 0 | 10 | 10 | 10 | 10 | 20 | 40 | 20 | 30 | 20 | 20 | 0 | 20 | 20 | 10 | 0 | 30 | 20 | 0 | 0 | 0 |
| Corn | 0 | 10 | 0 | 0 | 0 | 0 | 0 | 10 | 10 | 0 | 0 | 0 | 10 | 0 | 10 | 0 | 10 | 10 | 10 | 20 | 0 | 10 | 20 | 10 | 0 | 0 | 0 |
| Crabgrass, Large | 20 | 10 | 0 | 10 | 10 | 0 | 10 | 10 | 10 | 10 | 0 | 10 | 30 | 0 | 10 | 10 | 10 | 10 | 0 | 10 | 10 | 20 | 20 | 10 | 0 | 0 | 10 |
| Foxtail, Giant | 10 | 20 | 10 | 20 | 30 | 30 | 10 | 20 | 30 | 20 | 10 | 10 | 10 | 20 | 20 | 0 | 20 | 0 | 10 | 30 | 20 | 20 | 40 | 30 | 0 | 0 | 10 |
| Lambsquarters | 10 | 20 | 10 | 10 | 10 | 30 | 30 | 40 | 0 | 70 | 10 | 30 | 70 | 90 | 80 | 50 | 20 | 0 | 40 | 40 | 20 | 10 | 20 | 70 | 60 | 40 | 20 |
| Morningglory | 10 | 10 | 30 | 10 | 0 | 10 | 20 | 10 | 0 | 10 | 10 | 50 | 20 | 80 | — | 0 | 0 | 10 | 40 | 20 | 20 | 20 | 20 | 30 | 0 | 20 | 20 |
| Oat, Wild | 0 | 10 | 0 | 0 | 0 | 0 | 20 | 20 | 40 | 0 | 20 | 20 | 20 | 30 | 40 | 0 | 20 | 0 | 0 | 0 | 10 | 20 | 40 | 20 | 0 | 0 | 0 |
| Pigweed | 10 | 30 | 40 | 20 | 30 | 20 | 20 | 20 | 30 | 20 | 20 | 20 | 40 | 40 | 50 | 10 | 30 | 0 | 10 | 40 | 50 | 30 | 40 | 70 | 30 | 40 | 20 |
| Surinam Grass | 10 | 0 | 0 | 10 | 10 | 10 | 10 | 10 | 10 | 10 | 10 | 10 | 30 | 10 | 10 | 0 | 10 | 10 | 0 | 10 | 10 | 10 | 20 | 10 | 0 | 10 | 10 |
| Velvetleaf | 10 | 20 | — | 30 | — | 20 | 10 | 20 | 40 | 10 | 0 | 10 | 20 | 10 | 10 | 10 | 10 | 10 | 10 | 10 | 20 | 10 | 70 | 20 | 20 | 10 | 0 |
| Wheat | 10 | 0 | 0 | 0 | 0 | 0 | 0 | 0 | 30 | 0 | 0 | 10 | 10 | 20 | 20 | 0 | 0 | 0 | 0 | 0 | 0 | 0 | 30 | 10 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 380 | 381 | 382 | 383 | 384 | 385 | 386 | 387 | 388 | 389 | 390 | 391 | 392 | 393 | 394 | 395 | 396 | 397 | 398 | 399 | 400 | 401 | 402 | 403 | 404 | 405 | 406 |
| Barnyardgrass | 0 | 0 | 0 | 0 | 0 | 0 | 0 | 0 | 0 | 0 | 0 | 10 | 10 | 10 | 0 | 20 | 10 | 0 | 0 | 10 | 0 | 0 | 0 | 0 | 10 | 10 | 10 |
| Blackgrass | 20 | 20 | 10 | 0 | 20 | 10 | 0 | 0 | 0 | 0 | 0 | 30 | 20 | 20 | 0 | 20 | 10 | 0 | 0 | 30 | 0 | 0 | 0 | 0 | 10 | 0 | 10 |
| Cocklebur | 20 | 20 | 10 | 0 | 30 | 20 | 40 | 0 | 10 | 0 | 20 | 30 | 20 | 30 | 10 | 70 | 20 | 0 | 0 | 60 | 0 | 0 | 0 | 0 | 10 | 10 | 20 |
| Corn | 0 | 0 | 0 | 0 | 0 | 10 | 0 | 0 | 10 | 0 | 0 | 10 | 0 | 0 | 0 | 30 | 10 | 0 | 0 | 10 | 0 | 0 | 0 | 0 | 0 | 10 | 0 |
| Crabgrass, Large | 0 | 10 | 10 | 0 | 10 | 0 | 10 | 0 | 20 | 0 | 10 | 10 | 10 | 10 | 10 | 20 | 20 | 0 | 0 | 20 | 0 | 0 | 0 | 0 | 0 | 10 | 10 |
| Foxtail, Giant | 0 | 10 | 0 | 0 | 10 | 10 | 20 | 0 | 10 | 0 | 10 | 20 | 20 | 20 | 20 | 30 | 20 | 10 | 20 | 30 | 0 | 0 | 0 | 0 | 10 | 0 | 30 |
| Lambsquarters | 60 | 70 | 50 | 20 | 70 | 50 | 30 | 20 | 60 | 10 | 60 | 70 | 70 | 60 | 30 | 80 | 80 | 0 | 10 | 80 | 0 | 0 | 30 | 0 | 60 | 50 | 50 |
| Morningglory | 20 | 20 | 20 | 10 | 30 | 30 | 20 | 0 | 10 | 0 | 0 | 30 | 20 | 30 | 20 | 40 | 20 | 0 | 0 | 20 | 0 | 0 | 0 | 0 | 20 | 20 | 20 |
| Oat, Wild | 10 | 10 | 10 | 0 | 10 | 0 | 0 | 0 | 0 | 0 | 0 | 0 | 0 | 10 | 0 | 40 | 0 | 0 | 0 | 20 | 0 | 0 | 0 | 0 | 0 | 0 | 0 |
| Pigweed | 50 | 20 | 30 | 20 | 30 | 50 | 50 | 20 | 40 | 20 | 50 | 50 | 60 | 40 | 30 | 40 | 40 | 0 | 0 | 60 | 0 | 0 | 0 | 0 | 10 | 30 | 40 |
| Surinam Grass | 10 | 10 | 10 | 10 | 10 | 0 | 10 | 0 | 10 | 0 | 0 | 10 | 10 | 10 | 10 | 20 | 20 | 10 | 10 | 20 | 0 | 0 | 0 | 0 | 0 | 0 | 0 |
| Velvetleaf | 10 | 10 | 10 | 10 | 30 | 20 | 40 | 0 | 20 | 0 | 0 | 40 | 30 | 40 | 10 | 30 | 20 | 10 | 20 | 20 | 0 | 0 | 20 | 0 | 30 | 10 | 20 |
| Wheat | 10 | 10 | 0 | 0 | 10 | 0 | 0 | 0 | 0 | 0 | 0 | 0 | 0 | 0 | 0 | 20 | 10 | 0 | 0 | 10 | 0 | 0 | 0 | 0 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 407 | 408 | 409 | 410 | 411 | 412 | 413 | 414 | 415 | 416 | 417 | 418 | 419 | 420 | 421 | 422 | 423 | 424 | 425 | 426 | 427 | 428 | 429 | 430 | 431 | 432 | 433 |
| Barnyardgrass | 0 | 20 | 0 | 0 | 0 | 0 | 10 | 30 | 20 | 20 | 10 | 20 | 20 | 20 | 0 | 0 | 10 | 10 | 10 | 0 | 0 | 0 | 30 | 30 | 10 | 20 | 10 |
| Blackgrass | 0 | 40 | 10 | 10 | 40 | 40 | 10 | 40 | 30 | 20 | 10 | 10 | 10 | 10 | 20 | 20 | 30 | 0 | 0 | 0 | 0 | 0 | 20 | 40 | 30 | 40 | 30 |
| Cocklebur | 10 | 60 | 0 | 0 | 0 | 0 | 30 | 60 | 30 | 50 | 20 | 20 | 20 | 10 | 0 | 0 | 10 | 20 | 10 | 10 | 10 | 0 | 60 | 60 | 30 | 40 | 40 |
| Corn | 10 | 10 | 0 | 0 | 10 | 0 | 10 | 30 | 20 | 20 | 10 | 20 | 20 | 20 | 0 | 0 | 10 | 0 | 10 | 10 | 0 | 0 | 30 | 20 | 20 | 30 | 20 |
| Crabgrass, Large | 10 | 30 | 0 | 0 | 0 | 0 | 10 | 30 | 20 | 30 | 20 | 20 | 30 | 30 | 0 | 0 | 10 | 10 | 20 | 10 | 0 | 10 | 30 | 30 | 20 | 10 | 20 |
| Foxtail, Giant | 10 | 30 | 0 | 10 | 20 | 30 | 20 | 30 | 20 | 20 | 20 | 20 | 30 | 20 | 0 | 0 | 20 | 20 | 20 | 20 | 0 | 10 | 30 | 30 | 20 | 0 | 30 |
| Lambsquarters | — | 90 | 30 | 20 | 20 | 60 | 70 | 80 | 70 | 70 | 70 | 60 | 70 | 70 | 0 | 0 | 50 | 40 | 60 | 20 | 10 | 10 | 50 | 60 | 40 | 0 | 40 |
| Morningglory | 20 | 10 | 0 | 0 | 0 | 0 | 30 | 20 | 10 | 30 | 50 | 80 | 80 | 80 | 0 | 0 | 40 | 30 | 70 | 20 | 20 | 20 | 70 | 60 | 40 | 20 | 50 |
| Oat, Wild | 0 | 30 | 0 | 0 | 40 | 30 | 0 | 30 | 30 | 30 | 10 | 0 | 0 | 20 | 0 | 0 | 0 | 0 | 0 | 0 | 0 | 0 | 30 | 40 | 30 | 50 | 50 |
| Pigweed | 30 | 90 | 30 | 0 | 80 | 90 | 50 | 90 | 70 | 60 | 50 | 70 | 60 | 60 | 0 | 0 | 40 | 30 | 50 | 30 | 20 | 20 | 40 | 60 | 50 | 0 | 50 |
| Surinam Grass | 0 | 20 | 10 | 0 | 0 | 0 | 20 | 20 | 10 | 20 | 10 | 20 | 20 | 20 | 0 | 0 | 10 | 20 | 10 | 10 | 0 | 10 | 20 | 30 | 20 | 10 | 20 |
| Velvetleaf | 20 | 30 | 0 | 0 | 0 | 0 | 30 | 50 | 30 | 40 | 20 | 20 | 20 | 20 | 0 | 0 | 40 | 30 | 20 | 30 | 10 | 10 | 50 | 60 | 20 | 50 | 10 |
| Wheat | 0 | 20 | 0 | 0 | 20 | 10 | 0 | 10 | 20 | 20 | 10 | 10 | 10 | 10 | 0 | 0 | 0 | 0 | 0 | 0 | 0 | 0 | 20 | 30 | 30 | 50 | 40 |

| | 125 g ai/ha | | | | | | | | | | | | 62 g ai/ha | | | | | | | | | 31 g ai/ha | | | 16 g ai/ha |
|---|---|---|---|---|---|---|---|---|---|---|---|---|---|---|---|---|---|---|---|---|---|---|---|---|---|
| | Compounds | | | | | | | | | | | | Compounds | | | | | | | | | Compounds | | | Compound |
| | 434 | 435 | 436 | 437 | 438 | 439 | 440 | 441 | 442 | 443 | 444 | 445 | 54 | 72 | 73 | 74 | 129 | 152 | 191 | 192 | 251 | 49 | 168 | 346 | 251 |
| Barnyardgrass | 10 | 10 | 0 | 0 | 0 | 0 | 0 | 0 | 10 | 20 | 0 | 30 | 0 | 0 | 0 | 0 | 0 | 30 | 0 | 0 | 0 | 10 | 0 | 0 | 0 |
| Blackgrass | 30 | 20 | 0 | 0 | 20 | 0 | 20 | 20 | 30 | 30 | 0 | 40 | 0 | 40 | 30 | 30 | 0 | 20 | 0 | 0 | 50 | 0 | 0 | 30 | 10 |
| Cocklebur | 50 | 20 | 0 | 10 | 20 | 0 | 0 | 10 | 80 | 50 | 0 | 40 | 10 | 0 | 20 | 0 | 60 | 60 | 0 | 0 | 0 | 10 | 30 | 10 | 0 |
| Corn | 40 | 20 | 0 | 0 | 30 | 0 | 0 | 0 | 30 | 20 | 0 | 20 | 10 | 0 | 0 | 0 | — | 20 | 10 | 10 | 0 | 10 | 10 | 0 | 0 |
| Crabgrass, Large | 20 | 10 | 0 | 0 | 0 | 0 | 0 | 0 | 10 | 20 | 0 | 30 | 0 | 0 | 0 | 0 | 0 | 10 | 20 | 10 | 0 | 10 | 10 | 0 | 0 |
| Foxtail, Giant | 20 | 20 | 0 | 0 | 0 | 0 | 0 | 0 | 20 | 30 | 0 | 30 | 0 | 0 | 10 | 10 | 10 | 10 | 10 | 10 | 0 | 0 | 0 | 0 | 0 |
| Lambsquarters | 40 | 50 | 0 | 20 | 10 | 0 | 0 | 20 | 40 | 70 | 0 | 60 | 40 | 0 | 0 | 0 | 0 | 70 | 60 | 70 | 10 | 20 | 50 | 0 | 0 |
| Morningglory | 60 | 70 | 0 | 0 | 40 | 0 | 0 | 0 | 20 | 50 | 0 | 60 | 10 | 0 | 10 | 0 | — | 50 | 40 | 60 | 0 | 0 | 20 | 0 | 0 |
| Oat, Wild | 50 | 50 | 10 | — | 30 | 0 | 20 | 30 | 50 | 40 | 0 | 40 | 0 | 20 | 20 | 30 | 0 | 10 | 10 | 10 | 20 | 0 | 10 | 30 | 20 |
| Pigweed | 40 | 40 | 0 | 20 | 10 | 0 | 0 | 20 | 50 | 60 | 0 | 50 | 0 | 0 | 10 | 20 | 0 | 50 | 30 | 30 | 10 | 10 | 50 | 60 | 0 |
| Surinam Grass | 10 | 20 | 0 | 0 | 0 | 0 | 0 | 0 | 20 | 20 | 0 | 20 | 0 | 0 | 10 | 0 | 0 | 30 | 10 | 10 | 0 | 10 | 10 | 0 | 0 |
| Velvetleaf | 10 | 10 | 0 | 0 | 0 | 0 | 0 | 0 | 30 | 50 | 0 | 50 | 0 | 0 | 30 | 0 | 0 | 50 | 10 | 20 | 10 | 10 | 10 | 0 | 0 |
| Wheat | 40 | 20 | 10 | — | 20 | 0 | 0 | 20 | 20 | 40 | 0 | 30 | 0 | 10 | 20 | 20 | 0 | 10 | 0 | 0 | 20 | 0 | 0 | 10 | 20 |

Preemergence

| | 500 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 |
| Barnyardgrass | 100 | 100 | 100 | 100 | 90 | 100 | 90 | 0 | 100 | 100 | 100 | 40 | 100 | 100 | 100 | 100 | 100 | 90 | 70 | 70 | 80 | 0 | 0 | 30 | 0 | 0 | 100 |
| Cocklebur | 100 | 90 | 50 | 0 | 0 | 100 | 70 | 0 | 80 | 100 | 50 | 0 | — | 80 | 0 | 100 | 100 | 60 | 100 | 20 | 0 | 0 | 0 | 0 | — | 0 | 60 |
| Corn | 70 | 80 | 60 | 60 | 0 | 90 | 70 | 0 | 60 | 50 | 80 | 0 | 70 | 90 | 30 | 70 | 60 | 50 | 0 | 10 | 0 | 0 | 0 | 0 | 0 | 0 | 90 |
| Crabgrass, Large | 100 | 100 | 100 | 90 | 100 | 100 | 100 | 0 | 100 | 100 | 100 | 10 | 100 | 100 | 100 | 100 | 90 | 100 | 100 | 100 | 100 | 10 | 10 | 60 | 0 | 0 | 100 |
| Foxtail, Giant | 100 | 100 | 100 | 100 | 100 | 100 | 100 | 0 | 100 | 100 | 100 | 20 | 100 | 100 | 100 | 100 | 90 | 100 | 90 | 70 | 40 | 10 | 0 | 80 | 0 | 0 | 100 |
| Lambsquarters | 100 | 100 | 100 | 100 | 90 | 100 | 100 | 80 | 100 | 100 | 100 | 0 | 100 | 100 | 100 | 100 | 100 | 100 | 90 | 90 | 80 | 0 | 0 | 0 | 0 | — | 100 |
| Morningglory | 70 | 90 | 60 | 30 | 0 | — | 90 | 0 | 30 | 30 | 10 | 30 | 100 | 100 | 10 | 50 | 10 | 100 | 60 | 80 | 20 | 10 | 0 | 0 | 0 | 0 | 40 |
| Pigweed | 100 | 100 | 100 | 100 | 100 | 100 | 100 | 30 | 100 | 100 | 100 | 0 | 100 | 100 | 100 | 100 | 100 | 100 | 100 | 100 | 80 | 0 | 0 | 100 | 0 | 0 | 100 |
| Rice | 80 | 90 | 90 | 60 | 20 | 100 | 80 | 10 | 90 | 80 | 80 | 0 | 90 | 100 | 50 | 90 | 70 | 90 | 40 | 50 | 30 | 0 | 0 | 0 | 0 | 0 | — |
| Surinam Grass | 90 | 100 | 90 | 90 | 50 | 100 | 60 | 0 | 90 | 90 | 100 | 10 | 100 | 100 | 90 | 90 | 70 | 70 | 70 | 60 | 50 | 0 | 0 | 10 | 0 | 0 | 90 |
| Velvetleaf | 100 | 100 | 100 | 100 | 60 | 100 | 70 | 0 | 100 | 100 | 80 | 10 | 100 | 100 | 80 | 100 | 100 | 100 | 100 | 30 | 30 | 0 | 0 | 0 | 0 | 0 | 100 |
| Wheat | — | — | — | — | — | — | — | — | — | — | — | — | — | — | | | | | | | | | | | | | |
| | 500 g ai/ha | | | | | | | | | | | | | | | | | | | | | | | | | | |
| | Compounds | | | | | | | | | | | | | | | | | | | | | | | | | | |
| | 28 | 29 | 30 | 31 | 32 | 33 | 34 | 35 | 36 | 37 | 40 | 41 | 42 | 43 | 44 | 45 | 46 | 47 | 48 | 50 | 51 | 52 | 55 | 56 | 57 | 58 | 59 |
| Barnyardgrass | 90 | 100 | 90 | 90 | 60 | 90 | 80 | 100 | 80 | 60 | 40 | 80 | 30 | 100 | 100 | 100 | 100 | 90 | 90 | 80 | 100 | 60 | 100 | 80 | 70 | 100 | 70 |
| Cocklebur | 0 | 90 | 10 | 40 | 0 | 30 | 10 | 30 | 0 | 0 | 0 | 0 | 0 | 10 | 20 | 80 | 30 | 40 | 50 | 0 | 50 | 10 | 10 | 20 | 10 | 100 | 0 |
| Corn | 10 | 50 | 10 | 20 | 0 | 30 | 0 | 40 | 10 | 10 | 0 | 0 | 0 | 10 | 0 | 30 | 10 | 10 | 0 | 10 | 50 | 0 | 20 | 20 | 0 | 30 | 0 |
| Crabgrass, Large | 50 | 100 | 60 | 50 | 60 | 100 | 100 | 90 | 20 | 90 | 70 | 100 | 70 | 100 | 100 | 90 | 100 | 100 | 80 | 90 | 100 | 90 | 100 | 100 | 100 | 100 | 20 |
| Foxtail, Giant | 60 | 100 | 80 | 90 | 10 | 100 | 90 | 100 | 30 | 60 | 10 | 70 | 10 | 80 | 70 | 100 | 100 | 90 | 90 | 100 | 100 | 60 | 100 | 100 | 100 | 100 | 40 |
| Lambsquarters | 100 | 90 | 100 | 100 | 100 | 100 | 100 | 100 | 100 | 100 | 80 | 100 | 80 | 100 | 100 | 100 | 100 | 100 | 90 | 100 | 100 | 90 | 100 | 100 | 100 | 100 | 100 |
| Morningglory | 30 | 100 | 60 | 80 | 60 | — | 20 | 10 | 10 | 0 | 10 | 10 | 10 | 10 | 10 | 30 | 20 | 30 | 40 | 40 | 0 | 0 | 10 | 20 | 0 | 30 | 10 |
| Pigweed | 100 | 100 | 100 | 100 | 70 | 100 | 100 | 100 | 100 | 100 | 60 | 70 | 40 | 100 | 100 | 100 | 100 | 100 | 100 | 100 | 100 | 90 | 100 | 100 | 100 | 100 | 100 |
| Rice | — | | | | | | | | | | | | | | | — | — | — | 60 | 50 | 50 | 80 | 70 | 70 | — | 80 | 0 |
| Surinam Grass | 70 | 80 | 20 | 60 | 30 | 80 | 80 | 80 | 50 | 40 | 10 | 60 | 20 | 50 | 40 | 50 | 70 | 70 | 80 | 50 | 80 | 30 | 80 | 60 | 40 | 60 | 10 |
| Velvetleaf | 100 | 90 | 70 | 90 | 70 | 80 | 60 | 60 | 50 | 20 | 10 | 10 | 10 | 100 | 100 | 100 | 100 | 100 | 100 | 50 | 100 | 20 | 30 | 100 | 0 | 100 | 40 |
| | 500 g ai/ha | | | | | | | | | | | | | | | | | | | | | | | | | | |
| | Compounds | | | | | | | | | | | | | | | | | | | | | | | | | | |
| | 60 | 61 | 62 | 63 | 64 | 65 | 66 | 67 | 68 | 69 | 70 | 71 | 75 | 76 | 77 | 78 | 79 | 80 | 81 | 82 | 83 | 84 | 85 | 86 | 87 | 88 | 89 |
| Barnyardgrass | 100 | 60 | 80 | 100 | 90 | 90 | 30 | 80 | 10 | 0 | 50 | 0 | 90 | 100 | 70 | 0 | 10 | 20 | 60 | 0 | 0 | 0 | 0 | 0 | 70 | 90 | 0 |
| Cocklebur | 0 | 0 | 10 | 10 | 0 | 0 | 0 | 0 | 0 | — | 0 | 0 | 10 | 40 | 0 | 0 | 0 | 0 | 0 | 0 | 0 | — | 0 | 0 | 0 | 0 | 0 |
| Corn | 0 | 10 | 10 | 30 | 0 | 10 | 0 | — | 0 | 0 | 20 | 0 | 30 | 50 | 40 | 0 | 0 | 10 | 0 | 0 | 0 | 0 | 0 | 0 | 0 | 60 | 0 |
| Crabgrass, Large | 100 | 100 | 100 | 100 | 100 | 80 | 10 | 20 | 0 | 0 | 100 | 0 | 70 | 90 | 80 | 10 | 0 | 0 | 10 | 0 | 0 | 0 | 0 | 0 | 100 | 100 | 80 |
| Foxtail, Giant | 100 | 80 | 90 | 100 | 100 | 80 | 10 | 10 | 0 | 0 | 100 | 0 | 90 | 100 | 60 | 30 | 10 | 70 | 60 | 0 | 0 | 0 | 0 | 0 | 80 | 90 | 0 |
| Lambsquarters | 90 | 100 | 100 | 100 | 100 | 100 | 50 | 10 | 90 | 40 | 0 | 0 | 80 | 90 | 90 | 0 | 0 | 0 | 0 | 0 | 0 | 0 | 0 | 0 | 100 | 100 | 0 |
| Morningglory | 20 | 20 | 10 | 10 | 0 | 10 | 0 | 0 | 30 | 20 | 20 | 0 | 10 | 80 | 0 | 10 | 0 | 20 | 60 | 0 | 0 | 0 | 0 | — | 0 | 0 | 0 |
| Pigweed | 100 | 100 | 100 | 100 | 100 | 100 | 30 | 60 | 90 | 0 | 50 | 0 | 90 | 100 | 100 | 30 | 0 | 0 | 80 | 0 | 0 | 0 | 0 | 0 | 90 | 100 | 0 |
| Rice | 50 | 0 | 10 | 80 | 60 | 50 | 0 | 50 | 0 | 0 | 10 | 0 | 70 | 60 | 60 | — | — | — | 0 | 0 | 0 | 0 | 0 | 0 | 60 | 60 | 0 |
| Surinam Grass | 50 | 30 | 40 | 80 | 50 | 50 | 10 | 50 | 0 | 0 | 40 | 0 | 70 | 90 | 40 | 10 | 10 | 10 | 10 | 0 | 0 | 0 | 0 | 0 | 20 | 80 | 0 |
| Velvetleaf | 90 | 90 | 100 | 70 | 40 | 60 | 0 | 20 | 10 | 0 | 70 | 0 | 70 | 90 | 30 | 10 | 10 | 20 | 80 | 0 | 0 | 0 | 0 | 0 | 40 | 60 | 0 |
| | 500 g ai/ha | | | | | | | | | | | | | | | | | | | | | | | | | | |
| | Compounds | | | | | | | | | | | | | | | | | | | | | | | | | | |
| | 90 | 91 | 92 | 93 | 94 | 95 | 96 | 97 | 98 | 99 | 100 | 101 | 102 | 104 | 105 | 106 | 107 | 108 | 109 | 110 | 112 | 113 | 114 | 115 | 117 | 118 | 119 |
| Barnyardgrass | 20 | 10 | 0 | 20 | 20 | 80 | 90 | 0 | 0 | 30 | 0 | 70 | 90 | 80 | 30 | 70 | 0 | 90 | 100 | 10 | 70 | 90 | 80 | 100 | 90 | 90 | 10 |
| Cocklebur | 0 | — | 0 | 0 | 0 | 0 | 0 | 0 | 0 | 0 | 0 | 90 | 0 | 0 | 0 | 0 | 0 | 30 | 20 | 10 | 0 | 20 | 10 | 40 | 30 | 10 | 0 |
| Corn | 20 | 10 | 0 | 10 | 0 | 60 | 70 | 0 | 0 | 0 | 0 | 40 | 50 | 0 | 0 | 0 | 0 | 30 | 10 | 0 | 0 | 0 | 10 | 30 | 30 | 10 | 0 |
| Crabgrass, Large | 70 | — | 10 | 20 | 20 | 90 | 100 | 0 | 0 | 70 | 0 | 90 | 100 | 90 | 90 | 100 | 10 | 100 | 100 | 100 | 90 | 100 | 100 | 100 | 100 | 100 | 80 |
| Foxtail, Giant | 60 | 10 | 10 | 30 | 30 | 100 | 90 | 0 | 0 | 20 | 0 | 60 | 80 | 20 | 10 | 40 | 0 | 100 | 100 | 20 | 20 | 30 | 90 | 100 | 80 | 70 | 0 |
| Lambsquarters | 90 | 30 | 0 | 70 | 0 | 100 | 100 | 0 | 0 | 20 | 0 | 100 | 100 | 80 | 80 | 100 | 30 | 100 | 90 | 90 | 90 | 100 | 100 | 100 | 100 | 0 | 0 |
| Morningglory | 10 | 10 | 0 | 10 | 0 | — | 10 | 0 | 0 | 0 | 0 | 10 | 100 | 20 | 10 | 10 | 10 | 70 | 100 | 60 | 60 | 50 | — | 90 | 60 | 60 | 10 |
| Pigweed | 80 | 0 | 0 | 70 | 20 | 100 | 100 | 0 | 0 | — | 0 | 100 | 100 | 70 | 80 | 70 | 0 | 100 | 100 | 10 | 90 | 100 | 100 | 100 | 90 | 90 | 0 |
| Rice | 0 | 10 | 0 | 0 | 0 | 50 | 70 | 0 | 0 | 0 | 0 | 40 | — | — | — | — | — | | | | | | | | | | |
| Surinam Grass | 20 | — | 0 | 0 | 0 | 70 | 80 | 0 | 0 | 30 | 0 | 30 | 40 | 20 | 10 | 20 | 10 | 80 | 80 | 10 | 20 | 10 | 30 | 70 | 60 | 50 | 10 |
| Velvetleaf | 20 | — | 0 | 10 | 0 | 60 | 90 | 0 | 0 | 20 | 0 | 50 | 70 | 0 | 0 | 0 | 0 | 100 | 60 | 10 | 50 | 20 | 60 | 100 | 70 | 10 | 10 |
| | 500 g ai/ha | | | | | | | | | | | | | | | | | | | | | | | | | | |
| | Compounds | | | | | | | | | | | | | | | | | | | | | | | | | | |
| | 120 | 121 | 122 | 123 | 124 | 125 | 126 | 127 | 128 | 129 | 130 | 131 | 132 | 133 | 134 | 135 | 136 | 137 | 138 | 139 | 140 | 141 | 142 | 143 | 144 | 145 | 146 |
| Barnyardgrass | 90 | 80 | 90 | 0 | 10 | 0 | 60 | 70 | 90 | 60 | 70 | 90 | 50 | 60 | 80 | 50 | 100 | 70 | 70 | 90 | 80 | 0 | 100 | 0 | 100 | 100 | 90 |
| Cocklebur | 0 | 0 | 70 | 0 | 0 | 0 | 0 | 20 | 20 | 20 | 0 | 10 | 0 | 0 | 0 | 0 | 10 | 0 | 10 | 10 | 80 | 10 | 40 | 0 | 20 | 20 | 0 |
| Corn | 0 | 0 | 30 | 0 | 0 | 0 | 0 | 0 | 20 | 0 | 20 | 0 | 10 | 0 | 10 | 0 | 30 | 10 | 10 | 40 | 0 | 0 | 0 | 0 | 10 | 10 | 0 |
| Crabgrass, Large | 90 | 50 | 100 | 0 | 0 | 0 | 30 | 100 | 100 | 90 | 100 | 100 | 100 | 80 | 100 | 90 | 100 | 100 | 100 | 100 | 100 | 20 | 100 | 0 | 100 | 100 | 100 |
| Foxtail, Giant | 70 | 70 | 80 | 0 | 10 | 0 | 0 | 70 | 60 | 20 | 70 | 70 | 80 | 40 | 70 | 20 | 100 | 80 | 70 | 100 | 80 | 20 | 100 | 0 | 100 | 100 | 90 |
| Lambsquarters | 20 | 100 | 100 | 0 | 80 | 0 | 90 | 70 | 70 | 20 | 70 | 100 | 90 | 100 | 100 | 100 | 100 | 80 | 100 | 100 | 100 | 0 | 100 | 0 | 100 | 100 | 100 |
| Morningglory | — | 20 | 70 | 0 | 0 | — | 20 | — | 30 | 0 | 10 | 30 | 20 | 10 | 20 | — | 70 | 20 | 20 | 30 | — | 30 | 30 | 0 | 100 | 40 | 10 |
| Pigweed | 30 | 100 | 80 | 0 | 90 | 0 | 70 | 70 | 80 | 30 | 100 | 100 | 100 | 100 | 100 | 100 | 100 | 100 | 100 | 100 | 80 | 30 | 100 | 0 | 100 | 100 | 100 |
| Rice | | | | | — | — | — | — | — | — | 0 | 50 | 0 | 40 | 50 | 30 | 50 | 10 | 40 | 60 | 70 | 0 | 80 | — | 30 | 20 | 40 |
| Surinam Grass | 0 | 20 | 70 | 0 | 10 | 0 | 20 | 60 | 60 | 10 | 80 | 60 | 70 | 20 | 50 | 30 | 90 | 80 | 30 | 90 | 60 | 10 | 60 | 0 | 100 | 80 | 30 |
| Velvetleaf | 40 | 20 | 60 | 0 | 0 | 0 | 20 | 20 | 80 | 10 | 90 | 100 | 70 | 70 | 90 | 50 | 100 | 60 | 40 | 80 | 20 | 20 | 100 | 0 | 100 | 100 | 90 |
| | 500 g ai/ha | | | | | | | | | | | | | | | | | | | | | | | | | | |
| | Compounds | | | | | | | | | | | | | | | | | | | | | | | | | | |
| | 147 | 148 | 149 | 150 | 151 | 153 | 154 | 155 | 156 | 157 | 158 | 159 | 160 | 161 | 162 | 163 | 164 | 165 | 166 | 167 | 169 | 170 | 171 | 179 | 180 | 181 | 182 |
| Barnyardgrass | 90 | 10 | 90 | 80 | 10 | 90 | 80 | 90 | 100 | 0 | 100 | 90 | 100 | 100 | 100 | 90 | 0 | 0 | 0 | 0 | 10 | 30 | 0 | 0 | 0 | 90 | 80 |
| Cocklebur | 10 | 0 | 0 | 0 | 0 | 0 | 10 | 30 | 100 | 20 | 40 | 100 | 70 | 0 | 90 | 70 | 0 | 0 | 0 | — | 0 | 0 | 0 | 0 | 0 | 20 | 0 |
| Corn | 10 | 0 | 50 | 20 | 0 | 20 | 0 | 30 | 80 | 0 | 60 | 0 | 20 | 10 | 80 | 40 | 0 | 0 | 0 | 0 | 0 | 0 | 0 | 0 | 0 | 40 | 0 |
| Crabgrass, Large | 100 | 0 | 100 | 10 | 10 | 80 | 80 | 100 | 100 | 20 | 100 | 70 | 80 | 100 | 100 | 90 | 0 | 0 | 0 | 0 | 40 | 80 | 30 | 0 | 0 | 100 | 100 |
| Foxtail, Giant | 100 | 20 | 100 | 60 | 10 | 50 | 30 | 90 | 100 | 0 | 100 | 60 | 70 | 100 | 100 | 80 | 0 | 0 | 0 | 0 | 10 | 20 | 0 | 0 | 0 | 100 | 100 |
| Lambsquarters | 100 | — | 100 | 100 | 0 | 100 | 90 | 100 | 100 | 30 | 100 | 100 | 100 | 100 | 100 | 100 | 0 | 0 | 0 | 0 | 60 | 80 | 0 | 0 | 0 | 100 | 100 |
| Morningglory | 30 | 0 | 40 | 20 | 0 | 10 | 0 | 40 | 100 | 40 | 50 | 40 | 90 | 40 | 80 | 70 | 0 | 0 | 0 | 0 | 10 | 10 | — | 0 | 0 | 20 | 30 |
| Pigweed | 100 | 0 | 100 | 100 | 0 | 100 | 100 | 100 | 100 | 40 | 100 | 100 | 100 | 100 | 100 | 100 | 0 | 0 | 0 | 0 | 100 | 90 | 0 | 0 | 0 | 100 | 100 |
| Rice | 10 | 10 | 30 | 40 | 0 | 20 | 0 | 50 | 100 | 0 | 90 | 70 | 80 | 40 | 90 | 80 | — | 0 | 0 | 0 | 0 | 0 | 0 | — | — | 70 | 50 |
| Surinam Grass | 80 | 0 | 80 | 10 | 10 | 70 | 10 | 50 | 100 | 0 | 90 | 70 | 70 | 60 | 100 | 70 | 0 | 0 | 0 | 0 | 10 | 30 | 0 | 0 | 0 | 70 | 60 |
| Velvetleaf | 80 | 0 | 40 | 10 | 0 | 100 | 70 | 70 | 100 | 0 | 80 | 90 | 80 | 70 | 100 | 90 | 0 | 0 | 0 | 0 | 0 | 20 | 20 | 0 | 0 | 10 | 70 |
| | 500 g ai/ha | | | | | | | | | | | | | | | | | | | | | | | | | | |
| | Compounds | | | | | | | | | | | | | | | | | | | | | | | | | | |
| | 183 | 184 | 185 | 186 | 187 | 188 | 189 | 190 | 193 | 194 | 195 | 196 | 197 | 198 | 199 | 200 | 201 | 202 | 203 | 204 | 205 | 206 | 207 | 209 | 210 | 211 | 212 |
| Barnyardgrass | 70 | 60 | 70 | 10 | 90 | 100 | 30 | 30 | 100 | 90 | 90 | 40 | 10 | 90 | 90 | 20 | 10 | 90 | 10 | 0 | 90 | 70 | 90 | 90 | 90 | 50 | 90 |
| Cocklebur | 0 | 0 | 0 | 0 | 0 | 0 | 0 | 0 | 70 | 20 | 0 | 0 | 0 | 20 | 0 | 0 | 0 | 10 | 10 | 0 | 20 | 0 | 20 | 30 | 40 | 0 | 50 |
| Corn | 0 | 0 | 0 | 10 | 60 | 50 | 0 | 0 | 100 | 50 | 0 | 0 | 0 | 40 | 10 | 0 | 0 | 20 | 0 | 0 | 40 | 40 | 50 | 50 | 70 | 10 | 30 |
| Crabgrass, Large | 90 | 100 | 50 | 10 | 100 | 100 | 60 | 80 | 100 | 100 | 100 | 100 | 0 | 100 | 100 | 90 | 60 | 100 | 80 | 0 | 90 | 60 | 100 | 60 | 90 | 70 | 100 |
| Foxtail, Giant | 80 | 70 | 70 | 10 | 70 | 100 | 10 | 10 | 100 | 80 | 60 | 0 | 0 | 100 | 100 | 10 | 0 | 90 | 10 | 0 | 100 | 20 | 90 | 60 | 100 | 50 | 100 |
| Lambsquarters | 100 | 80 | 80 | 0 | 40 | 100 | 30 | 50 | 100 | — | 80 | 0 | 0 | 100 | 90 | 80 | 30 | 100 | 30 | 0 | 100 | 30 | 100 | 100 | 100 | 80 | 100 |
| Morningglory | 10 | 50 | 50 | 0 | 30 | — | 20 | 0 | 100 | 50 | 40 | 0 | 0 | 80 | 60 | 10 | 10 | 30 | 10 | 10 | 30 | 20 | 40 | 20 | 30 | 10 | 60 |
| Pigweed | 100 | 100 | 100 | 0 | 60 | 70 | 100 | 90 | 100 | 100 | 90 | 0 | 0 | 100 | 100 | 90 | 20 | 90 | 90 | 10 | 100 | 80 | 100 | 100 | 100 | 30 | 100 |
| Rice | 80 | 0 | 0 | 20 | 80 | 70 | 50 | 30 | 100 | 70 | 20 | — | 0 | 80 | 20 | 10 | 0 | 30 | 0 | 0 | 70 | 30 | 70 | 70 | 80 | 10 | 20 |
| Surinam Grass | 50 | 30 | 20 | 10 | 70 | 70 | 20 | 10 | 100 | 60 | 30 | 0 | 20 | 50 | 70 | 10 | 0 | 60 | 0 | 0 | 80 | 20 | 60 | 50 | 70 | 30 | 70 |
| Velvetleaf | 50 | 50 | 50 | 20 | 20 | 100 | 20 | 10 | 100 | 60 | 20 | 20 | 0 | 60 | 60 | 20 | 10 | 20 | 20 | 0 | 80 | 10 | 60 | 50 | 70 | 10 | 80 |
| | 500 g ai/ha | | | | | | | | | | | | | | | | | | | | | | | | | | |
| | Compounds | | | | | | | | | | | | | | | | | | | | | | | | | | |
| | 213 | 214 | 215 | 216 | 217 | 218 | 219 | 220 | 221 | 222 | 223 | 224 | 225 | 226 | 227 | 228 | 229 | 230 | 231 | 232 | 233 | 234 | 235 | 236 | 237 | 238 | 239 |
| Barnyardgrass | 80 | 20 | 60 | 40 | 0 | 0 | 20 | 0 | 90 | 100 | 80 | 90 | 90 | 60 | 60 | 100 | 90 | 50 | 80 | 60 | 10 | 0 | 50 | 90 | 50 | 0 | 60 |
| Cocklebur | 0 | 0 | 0 | 0 | 0 | 0 | 0 | 0 | 0 | 80 | 20 | 50 | 40 | 0 | 0 | 80 | 40 | 0 | 30 | 0 | 0 | 0 | 0 | 20 | 0 | 0 | 0 |
| Corn | 10 | 0 | 0 | 0 | 0 | 0 | 0 | 0 | 0 | 90 | 80 | 70 | 70 | 20 | 50 | 80 | 40 | 10 | 50 | 0 | 0 | — | 20 | 10 | 20 | 0 | 0 |
| Crabgrass, Large | 70 | 70 | 80 | 40 | 0 | 20 | 100 | 100 | 100 | 100 | 100 | 100 | 100 | 100 | 100 | 100 | 100 | 70 | 90 | 100 | 90 | 40 | 90 | 70 | 90 | 0 | 100 |
| Foxtail, Giant | 90 | 90 | 80 | 60 | 0 | 0 | 0 | 10 | 70 | 90 | 80 | 70 | 100 | 70 | 70 | 100 | 80 | 80 | 90 | 30 | 0 | 10 | 50 | 50 | 40 | 0 | 0 |
| Lambsquarters | 100 | 80 | 100 | 100 | 0 | 0 | 0 | 70 | 100 | 100 | 100 | 100 | — | 90 | 90 | 100 | 100 | 90 | 100 | 90 | 30 | 50 | 90 | 100 | 90 | 0 | 70 |
| Morningglory | 20 | 0 | 20 | 0 | 0 | 0 | — | 0 | 0 | 100 | 30 | 30 | 10 | 10 | 20 | 40 | 20 | 10 | 10 | 20 | 0 | 0 | 10 | 20 | 20 | 0 | 0 |
| Pigweed | 100 | 100 | 100 | 90 | 0 | 0 | 80 | 100 | 100 | 100 | 100 | 100 | 100 | 70 | 100 | 100 | 100 | 80 | 100 | 90 | 60 | 40 | 100 | 90 | 70 | 0 | 70 |
| Rice | 40 | 0 | 0 | 70 | 0 | 0 | 0 | 0 | 20 | 100 | 30 | 80 | 90 | 30 | 70 | 90 | 70 | 0 | 50 | 50 | 0 | 0 | 20 | 60 | 30 | 0 | 0 |
| Surinam Grass | 40 | 30 | 70 | 70 | 0 | 0 | 0 | 10 | 30 | 100 | 90 | 100 | 90 | 40 | 70 | 100 | 80 | 10 | 60 | 30 | 20 | 0 | 60 | 60 | 30 | 0 | 0 |
| Velvetleaf | 80 | 0 | 60 | 0 | 0 | 0 | 10 | 0 | 50 | 100 | 100 | 100 | 100 | 50 | 100 | 100 | 100 | 30 | 90 | 40 | 10 | 0 | 30 | 80 | 60 | 0 | 0 |
| | 500 g ai/ha | | | | | | | | | | | | | | | | | | | | | | | | | | |
| | Compounds | | | | | | | | | | | | | | | | | | | | | | | | | | |
| | 240 | 241 | 242 | 243 | 244 | 245 | 246 | 247 | 248 | 249 | 250 | 252 | 253 | 254 | 256 | 257 | 258 | 259 | 260 | 261 | 262 | 263 | 264 | 265 | 266 | 267 | 268 |
| Barnyardgrass | 70 | 80 | 100 | 90 | 90 | 100 | 90 | 80 | 20 | 0 | 10 | 70 | 10 | 40 | 70 | 20 | 90 | 70 | 70 | 20 | 10 | 40 | 10 | 0 | 80 | 70 | 100 |
| Cocklebur | 0 | 0 | 40 | 20 | 20 | 0 | 0 | 0 | 0 | 0 | 0 | 0 | 0 | 0 | 0 | 0 | 0 | 0 | 0 | 0 | — | — | 0 | 0 | 10 | 0 | 90 |
| Corn | 0 | 30 | 60 | 50 | 60 | 10 | 20 | 30 | 0 | 0 | 0 | 60 | 0 | 0 | 0 | 0 | 0 | 0 | 0 | 0 | 0 | 0 | 0 | 0 | 90 | 20 | 90 |
| Crabgrass, Large | 100 | 100 | 100 | 100 | 100 | 100 | 100 | 80 | 40 | 0 | 10 | 100 | — | 100 | 100 | — | 50 | 50 | — | 50 | 30 | 60 | 50 | — | 100 | 100 | 100 |
| Foxtail, Giant | 80 | 90 | 90 | 80 | 90 | 90 | 90 | 70 | 60 | 0 | 10 | 90 | 30 | 30 | 60 | 50 | 20 | 70 | 30 | 0 | 0 | 20 | 10 | 50 | 100 | 80 | 100 |
| Lambsquarters | 80 | 100 | 100 | 100 | 100 | 100 | 100 | 100 | — | — | — | 100 | — | 100 | 100 | — | 100 | — | 100 | — | 90 | 100 | 100 | 0 | 100 | — | 100 |
| Morningglory | 0 | 0 | 10 | 0 | 20 | 20 | 10 | 10 | 0 | 10 | 0 | 0 | 0 | 10 | 0 | 0 | 10 | 90 | 0 | 0 | 0 | 0 | 0 | 0 | 10 | 30 | 20 |
| Pigweed | 100 | 100 | 100 | 100 | 100 | 100 | 100 | 100 | — | 0 | — | 100 | — | 100 | 100 | — | 100 | 100 | 100 | — | 100 | 100 | 100 | 0 | 100 | — | 100 |
| Rice | 0 | 80 | 80 | 80 | 80 | 50 | 70 | 40 | 0 | 0 | 10 | 50 | 0 | 20 | 80 | 0 | 90 | 0 | 30 | 60 | 0 | 10 | 0 | 0 | 80 | 40 | 90 |
| Surinam Grass | 20 | 90 | 90 | 80 | 90 | 80 | 80 | 50 | 60 | 20 | 30 | 70 | 30 | 40 | 60 | 0 | 30 | 30 | 10 | 0 | 10 | 30 | 10 | 40 | 90 | 50 | 100 |
| Velvetleaf | — | 100 | 100 | 100 | 100 | 90 | 90 | 100 | 20 | 0 | 20 | 60 | 20 | 20 | 80 | 0 | 60 | 0 | 10 | 20 | 80 | 90 | 10 | 10 | 100 | 50 | 100 |
| | 500 g ai/ha | | | | | | | | | | | | | | | | | | | | | | | | | | |
| | Compounds | | | | | | | | | | | | | | | | | | | | | | | | | | |
| | 269 | 270 | 271 | 272 | 273 | 274 | 275 | 276 | 277 | 278 | 279 | 280 | 281 | 282 | 283 | 284 | 285 | 286 | 287 | 288 | 289 | 290 | 291 | 292 | 293 | 294 | 295 |
| Barnyardgrass | 100 | 50 | 0 | 90 | 90 | 100 | 60 | 80 | 0 | 80 | 40 | 10 | 90 | 50 | 10 | 80 | 90 | 90 | 100 | 70 | 0 | 80 | 0 | 90 | 100 | 100 | 20 |
| Cocklebur | 70 | 0 | 0 | 0 | 0 | 0 | 0 | 30 | 10 | 0 | 0 | 0 | 0 | 0 | 0 | 0 | 0 | 0 | 60 | 0 | 0 | 0 | 0 | 40 | 90 | 100 | 0 |
| Corn | 80 | 50 | 10 | 10 | 30 | 80 | 20 | 60 | 0 | 20 | 0 | 0 | 0 | 0 | 0 | 0 | 40 | 50 | 30 | 0 | 0 | 0 | 0 | 80 | 100 | 100 | 10 |
| Crabgrass, Large | 100 | 20 | 0 | 100 | 100 | 100 | 0 | 70 | 0 | 80 | 80 | 10 | 70 | 80 | 50 | 90 | 70 | 100 | 100 | 0 | 20 | 90 | 0 | 100 | 100 | 100 | 80 |
| Foxtail, Giant | 100 | 50 | 0 | 90 | 90 | 90 | 20 | 90 | 0 | 80 | 30 | 10 | 60 | 20 | 10 | 10 | 80 | 90 | 90 | 10 | 0 | 80 | 0 | 70 | 100 | 100 | 70 |
| Lambsquarters | 100 | — | 60 | 100 | 100 | 100 | 100 | 100 | 80 | 100 | 100 | 80 | 100 | 100 | 100 | 100 | 100 | 100 | 100 | 90 | 100 | 100 | 0 | 100 | 100 | 100 | — |
| Morningglory | 20 | 0 | 0 | 30 | 10 | 10 | 40 | 10 | 0 | 10 | 0 | 0 | 20 | 0 | 0 | 0 | 10 | 0 | 10 | 0 | 0 | 0 | 0 | 30 | 100 | 20 | 20 |
| Pigweed | 100 | — | 20 | 100 | 100 | 100 | 40 | 100 | 100 | 100 | 100 | 100 | 100 | 100 | 100 | 100 | 100 | 100 | 100 | 90 | 100 | 100 | 0 | 100 | 100 | 100 | — |
| Rice | 90 | 60 | 0 | 20 | 40 | 80 | 60 | 80 | 20 | 80 | 50 | 10 | 60 | 40 | 0 | 20 | 80 | 80 | 90 | 70 | 0 | 80 | 0 | 90 | 100 | 100 | 20 |
| Surinam Grass | 100 | 20 | 0 | 70 | 50 | 80 | 10 | 60 | 0 | 60 | 40 | 10 | 40 | 20 | 0 | 10 | 70 | 60 | 80 | 0 | 0 | 10 | 0 | 100 | 100 | 100 | 20 |
| Velvetleaf | 100 | 70 | 0 | 50 | 40 | 70 | 0 | 50 | 40 | 60 | 20 | 20 | 70 | 30 | 0 | 60 | 100 | 60 | 100 | 0 | 0 | 0 | 0 | 70 | 100 | 100 | 60 |
| | 500 g ai/ha | | | | | | | | | | | | | | | | | | | | | | | | | | |
| | Compounds | | | | | | | | | | | | | | | | | | | | | | | | | | |
| | 296 | 297 | 298 | 299 | 300 | 301 | 302 | 303 | 304 | 305 | 306 | 307 | 308 | 309 | 310 | 311 | 312 | 313 | 314 | 315 | 316 | 317 | 318 | 319 | 329 | 330 | 331 |
| Barnyardgrass | 60 | 20 | 90 | 100 | 90 | 100 | 60 | 90 | 70 | 90 | 90 | 100 | 80 | 100 | 90 | 0 | 0 | 90 | 90 | 20 | 0 | 0 | 90 | 80 | 0 | 0 | 70 |
| Cocklebur | 0 | 0 | 0 | 60 | 20 | 0 | 0 | 20 | 0 | 70 | 50 | 100 | 0 | 80 | 50 | 0 | 0 | 10 | 0 | 0 | 0 | 0 | 0 | 10 | 0 | 0 | 0 |
| Corn | 40 | 0 | 70 | 90 | 70 | 10 | 10 | 70 | — | 60 | 100 | 80 | 70 | 90 | 70 | 0 | 0 | 30 | 10 | 0 | 0 | 0 | 0 | 40 | 0 | 0 | 40 |
| Crabgrass, Large | 100 | 80 | 100 | 100 | 100 | 100 | 100 | 100 | 100 | 100 | 100 | 100 | 70 | 100 | 100 | 0 | 0 | 90 | 30 | 20 | 20 | 0 | 90 | 100 | 0 | 0 | 80 |
| Foxtail, Giant | 90 | 60 | 100 | 100 | 100 | 90 | 30 | 100 | 90 | 100 | 100 | 90 | 90 | 100 | 100 | 0 | 0 | 100 | 100 | 20 | 0 | 0 | 80 | 50 | 0 | 0 | 80 |
| Lambsquarters | 100 | 80 | 100 | 100 | 100 | 100 | 100 | 100 | 100 | 100 | 100 | 100 | 100 | 90 | 100 | 0 | 0 | 100 | 80 | 80 | 60 | 0 | 100 | 100 | — | 0 | 100 |
| Morningglory | 20 | 10 | 40 | 100 | 60 | 80 | 10 | 50 | 10 | 0 | 10 | 10 | 10 | 0 | 10 | 0 | 0 | 40 | 20 | 0 | 0 | 0 | 30 | 20 | 0 | 0 | 0 |
| Pigweed | 80 | 40 | 100 | 100 | 100 | 100 | 100 | 100 | 100 | 100 | 100 | 100 | 100 | 100 | 100 | 0 | 0 | 100 | 100 | 100 | 90 | 0 | 100 | 100 | 0 | 0 | 50 |
| Rice | 40 | 10 | 60 | 90 | 70 | 70 | 10 | 80 | 20 | 80 | 80 | 90 | 50 | 90 | 80 | — | — | 40 | 10 | 0 | 0 | 0 | 50 | 70 | 0 | 0 | 40 |
| Surinam Grass | 60 | 20 | 100 | 100 | 100 | 100 | 30 | 100 | 70 | 100 | 90 | 90 | 60 | 100 | 70 | 0 | 0 | 80 | 80 | 10 | 0 | 0 | 40 | 60 | 0 | 0 | 60 |
| Velvetleaf | 80 | 10 | 60 | 100 | 100 | 100 | 80 | 100 | 100 | 100 | 100 | 100 | 100 | 100 | 100 | 0 | 0 | 100 | 100 | 20 | 20 | 0 | — | 30 | 0 | 0 | 80 |
| Wheat | | | | | | | | | | | | | | | | | | | | | | | | | — | — | — |
| | 500 g ai/ha | | | | | | | | | | | | | | | | | | | | | | | | | | |
| | Compounds | | | | | | | | | | | | | | | | | | | | | | | | | | |
| | 332 | 333 | 334 | 335 | 336 | 338 | 340 | 341 | 342 | 343 | 344 | 345 | 347 | 348 | 349 | 350 | 351 | 352 | 353 | 354 | 355 | 356 | 357 | 358 | 359 | 360 | 361 |
| Barnyardgrass | 60 | 20 | 100 | 60 | 100 | 80 | 100 | 100 | 100 | 100 | 70 | 30 | 0 | 0 | 100 | 0 | 0 | 90 | 90 | 60 | 90 | 80 | 80 | 100 | 90 | 40 | 100 |
| Cocklebur | 0 | 0 | 100 | 10 | 40 | 0 | 50 | 100 | 90 | 90 | 70 | 10 | 0 | 0 | 80 | 0 | 0 | 0 | 20 | 0 | 10 | 20 | 30 | 90 | 30 | 0 | 50 |
| Corn | 0 | 0 | 70 | 50 | 100 | 10 | — | — | — | — | 90 | 10 | 0 | 0 | 80 | 0 | 0 | 80 | — | 20 | 60 | 50 | 80 | 100 | 60 | 40 | 70 |
| Crabgrass, Large | 100 | 70 | 100 | 100 | 100 | 100 | 100 | 100 | 100 | 100 | — | 90 | 0 | 0 | 100 | 0 | 0 | 100 | 80 | 80 | 100 | 90 | 100 | 100 | 100 | 80 | 100 |
| Foxtail, Giant | 60 | 20 | 100 | 90 | 100 | 100 | 100 | 90 | 90 | 100 | 60 | 30 | 0 | 0 | 100 | 0 | 0 | 100 | 50 | 70 | 90 | 80 | 100 | 100 | 100 | 90 | 100 |
| Lambsquarters | 100 | 100 | 100 | 10 | 100 | 100 | 100 | 100 | 100 | 100 | 100 | 100 | 0 | 0 | 100 | 0 | 0 | 100 | — | 100 | 100 | 100 | 100 | 100 | 100 | 40 | 100 |
| Morningglory | 30 | 10 | 70 | 10 | 70 | 10 | — | 30 | 70 | 100 | 80 | 50 | 0 | 0 | 30 | 0 | 0 | 0 | 20 | 0 | 10 | 10 | 70 | 100 | 50 | 10 | 60 |
| Pigweed | 100 | 100 | 100 | 10 | 100 | 100 | 100 | 100 | 100 | 100 | 90 | 100 | 0 | 0 | 100 | 0 | 0 | 70 | — | 90 | 100 | 100 | 100 | 100 | 100 | 50 | 100 |
| Rice | 40 | 0 | — | — | — | — | — | — | — | — | — | — | — | — | — | — | 0 | — | — | — | — | — | — | — | — | | |
| Surinam Grass | 20 | 20 | 100 | 40 | 100 | 60 | 100 | 100 | 100 | 100 | 100 | 70 | 0 | 0 | 90 | 0 | 0 | 100 | 30 | 20 | 80 | 60 | 80 | 100 | 70 | 50 | 90 |
| Velvetleaf | 80 | 80 | 100 | 70 | 100 | 80 | 100 | 100 | 100 | 100 | 100 | 40 | 0 | 0 | 100 | 0 | 0 | 100 | 60 | 50 | 0 | 70 | 60 | 100 | 50 | 40 | 80 |
| Wheat | — | — | 90 | 30 | 100 | 20 | 100 | 100 | 100 | 100 | 80 | 20 | 0 | 0 | 90 | 0 | — | 70 | 20 | 10 | 30 | 20 | 90 | 90 | 70 | 40 | 80 |
| | 500 g ai/ha | | | | | | | | | | | | | | | | | | | | | | | | | | |
| | Compounds | | | | | | | | | | | | | | | | | | | | | | | | | | |
| | 362 | 363 | 364 | 365 | 366 | 368 | 369 | 370 | 371 | 372 | 373 | 374 | 375 | 376 | 377 | 378 | 379 | 380 | 381 | 382 | 383 | 384 | 385 | 386 | 387 | 388 | 389 |
| Barnyardgrass | 70 | 50 | 100 | 100 | 90 | 70 | 60 | 40 | 20 | 100 | 10 | 10 | 70 | 100 | 60 | 100 | 50 | 90 | 30 | 90 | 70 | 100 | 100 | 100 | 0 | 100 | 0 |
| Cocklebur | 10 | 10 | 100 | 60 | 20 | 0 | 0 | 0 | 0 | 50 | 0 | 0 | 0 | 0 | — | 0 | 0 | 10 | 0 | 20 | 0 | 70 | 0 | 90 | 0 | 0 | 0 |
| Corn | 60 | 30 | 100 | 100 | 70 | 40 | 60 | 20 | 0 | 80 | 0 | 10 | 10 | 70 | 30 | 70 | 0 | 80 | 10 | 90 | 50 | 90 | 80 | 90 | 0 | 80 | 0 |
| Crabgrass, Large | 100 | 90 | 100 | 100 | 100 | 80 | 70 | 50 | 50 | 100 | 10 | 30 | 100 | 100 | 90 | 80 | 80 | 100 | 70 | 100 | 90 | 100 | 90 | 100 | 20 | 100 | 20 |
| Foxtail, Giant | 80 | 90 | 100 | 100 | 90 | 50 | 50 | 20 | 20 | 100 | 20 | 20 | 80 | 90 | 40 | 70 | 20 | 100 | 50 | 100 | 70 | 100 | 80 | 100 | 10 | 90 | 0 |
| Lambsquarters | 70 | 80 | 100 | 100 | 90 | 100 | 100 | 100 | — | 100 | 20 | 70 | 80 | 100 | 90 | 100 | 90 | 100 | 90 | 100 | 100 | 100 | 100 | 100 | 0 | 90 | 0 |
| Morningglory | 10 | 0 | 100 | 90 | 50 | 0 | 10 | 10 | 0 | 10 | 0 | 0 | 10 | 20 | 40 | 80 | 20 | 60 | 20 | 90 | 10 | 100 | 60 | 100 | 0 | 20 | 0 |
| Pigweed | 100 | 60 | 100 | 100 | 100 | 100 | 100 | 80 | 50 | 100 | 30 | 100 | 100 | 100 | 90 | 100 | 60 | 100 | 70 | 100 | 100 | 100 | 90 | 100 | 0 | 100 | 0 |
| Surinam Grass | 50 | 60 | 100 | 100 | 80 | 30 | 20 | 10 | 10 | 80 | 20 | 30 | 50 | 80 | 60 | 80 | 30 | 80 | 60 | 90 | 50 | 100 | 90 | 100 | 10 | 100 | 0 |
| Velvetleaf | 20 | 30 | 100 | 100 | 60 | 20 | 0 | 0 | 0 | 80 | 0 | 30 | 100 | 50 | 50 | 100 | 20 | 70 | 10 | 70 | 10 | 100 | 70 | 100 | 0 | 70 | 0 |
| Wheat | 30 | 30 | 100 | 100 | 90 | 10 | 0 | 0 | 0 | 90 | 0 | 0 | 30 | 90 | 20 | 100 | 20 | 100 | 20 | 80 | 10 | 100 | 80 | 100 | 0 | 90 | 0 |
| | 500 g ai/ha | | | | | | | | | | | | | | | | | | | | | | | | | | |
| | Compounds | | | | | | | | | | | | | | | | | | | | | | | | | | |
| | 390 | 391 | 392 | 393 | 394 | 395 | 396 | 397 | 398 | 399 | 400 | 401 | 402 | 403 | 404 | 405 | 406 | 407 | 408 | 409 | 410 | 411 | 412 | 413 | 414 | 415 | 416 |
| Barnyardgrass | 50 | 100 | 100 | 100 | 90 | 100 | 100 | 0 | 0 | 100 | 10 | 0 | 10 | 0 | 100 | 90 | 90 | 100 | 80 | 100 | 100 | 80 | 80 | 70 | 100 | 80 | 80 |
| Cocklebur | 0 | 100 | 80 | 80 | 60 | 100 | 80 | 0 | 0 | 90 | 0 | 0 | 0 | 0 | 50 | 30 | 30 | 40 | 20 | 70 | 50 | 0 | 0 | 0 | 40 | 0 | 10 |
| Corn | 0 | 100 | 100 | 90 | 40 | 100 | 90 | 0 | 0 | 90 | 0 | 0 | 0 | 0 | 90 | 40 | 60 | 70 | 30 | 90 | 80 | 60 | 30 | — | 90 | 40 | 60 |
| Crabgrass, Large | 70 | 100 | 100 | 100 | 100 | 100 | 100 | 0 | 10 | 100 | 80 | 10 | 70 | 40 | 100 | 90 | 100 | 100 | 100 | 100 | 100 | 100 | 90 | 80 | 100 | 100 | 100 |
| Foxtail, Giant | 20 | 100 | 100 | 100 | 80 | 100 | 100 | 0 | 10 | 100 | 0 | 0 | 10 | 0 | 100 | 40 | 100 | 90 | 80 | 100 | 100 | 80 | 80 | 80 | 100 | 90 | 90 |
| Lambsquarters | — | 100 | 100 | 100 | 100 | 100 | 100 | 0 | 0 | 100 | 80 | 0 | — | 50 | 100 | 100 | 100 | 100 | 100 | 100 | 100 | 100 | 100 | 100 | 100 | 100 | 100 |
| Morningglory | 20 | 100 | 100 | 100 | 50 | 100 | 100 | 0 | 0 | 40 | 10 | 0 | 0 | 0 | 100 | 90 | 100 | 80 | 30 | 100 | 90 | 20 | 0 | 10 | 90 | 20 | 10 |
| Pigweed | 50 | 100 | 100 | 100 | 100 | 100 | 100 | 0 | 0 | 100 | 80 | 0 | 70 | 60 | 100 | 90 | 100 | 100 | 100 | 100 | 100 | 100 | 100 | 100 | 100 | 80 | 100 |
| Surinam Grass | 20 | 100 | 90 | 100 | 70 | 100 | 100 | 0 | 0 | 100 | 20 | 0 | 0 | 0 | 90 | 40 | 60 | 90 | 60 | 100 | 100 | 60 | 50 | 70 | 100 | 90 | 100 |
| Velvetleaf | 10 | 100 | 90 | 100 | 60 | 100 | 100 | 20 | 30 | 100 | 60 | 0 | 0 | 30 | 70 | 40 | 50 | 70 | 80 | 100 | 70 | 10 | 50 | 20 | 100 | 50 | 70 |
| Wheat | 10 | 100 | 100 | 100 | 20 | 100 | 100 | 0 | 0 | 100 | 0 | 0 | 10 | 0 | 100 | 20 | 40 | 70 | 80 | 100 | 90 | 60 | 20 | 80 | 100 | 30 | 60 |
| | 500 g ai/ha | | | | | | | | | | | | | | | | | | | | | | | | | | |
| | Compounds | | | | | | | | | | | | | | | | | | | | | | | | | | |
| | 417 | 418 | 419 | 420 | 421 | 422 | 423 | 424 | 425 | 426 | 427 | 428 | 429 | 430 | 431 | 432 | 433 | 434 | 435 | 436 | 437 | 438 | 439 | 440 | 441 | 442 | 443 |
| Barnyardgrass | 100 | 100 | 100 | 100 | 0 | 0 | 100 | 70 | 60 | 80 | 0 | 0 | 100 | 100 | 80 | 30 | 90 | 100 | 70 | 70 | 10 | 10 | 0 | 10 | 40 | 100 | 100 |
| Cocklebur | 10 | 40 | 70 | 40 | 0 | 0 | 50 | 0 | 0 | 0 | 0 | 0 | 90 | 70 | 0 | 40 | 40 | 70 | 10 | 0 | 0 | 0 | 0 | 0 | 0 | 10 | 30 |
| Corn | 90 | 100 | 90 | 90 | 0 | 0 | 100 | 30 | 70 | 10 | 0 | — | 100 | 80 | 0 | 0 | 30 | 70 | 0 | 30 | 0 | 10 | 0 | 0 | 0 | 50 | 80 |
| Crabgrass, Large | 100 | 100 | 100 | 100 | 0 | 0 | 100 | 100 | 100 | 100 | 30 | 40 | 100 | 100 | 90 | 90 | 100 | 100 | 100 | 90 | 90 | 40 | 0 | 0 | 100 | 100 | 100 |
| Foxtail, Giant | 100 | 100 | 100 | 100 | 0 | 0 | 100 | 100 | 90 | 20 | 0 | 10 | 100 | 100 | 70 | 80 | 100 | 100 | 60 | 70 | 20 | 30 | 0 | 20 | 60 | 70 | 100 |
| Lambsquarters | 100 | 100 | 100 | 100 | 0 | 0 | 100 | 100 | 90 | 90 | 40 | 50 | 100 | 100 | 100 | 0 | 100 | 100 | 100 | 90 | 60 | 0 | 0 | — | 100 | 100 | 100 |
| Morningglory | 50 | 100 | 100 | 100 | 0 | 0 | 100 | 60 | 40 | 40 | 0 | 0 | 100 | 70 | 20 | 30 | 100 | 100 | 40 | 20 | 10 | 0 | 0 | 20 | 30 | 20 | 30 |
| Pigweed | 100 | 100 | 100 | 100 | 0 | 0 | 100 | 100 | 100 | 100 | 50 | 70 | 100 | 100 | 100 | 0 | 100 | 100 | 100 | 90 | 80 | 0 | 0 | 90 | 100 | 100 | 100 |
| Surinam Grass | 90 | 100 | 100 | 100 | 0 | 0 | 100 | 100 | 100 | 90 | 0 | 10 | 100 | 100 | 50 | 60 | 60 | 60 | 70 | 70 | 30 | 10 | 0 | 10 | 30 | 50 | 80 |
| Velvetleaf | 100 | 80 | 100 | 100 | 0 | 0 | 100 | 40 | 70 | 70 | 10 | 10 | 100 | 100 | 60 | 80 | 70 | 70 | 20 | 50 | 0 | 0 | 0 | 0 | 20 | 50 | 90 |
| Wheat | 90 | 100 | 100 | 100 | 0 | 0 | 100 | 50 | 30 | 10 | 0 | 0 | 100 | 100 | 10 | 0 | 50 | 90 | 10 | 20 | 0 | 0 | 0 | 0 | 30 | 70 | 100 |
| | 500 g ai/ha | | 250 g ai/ha | | | | | | | 125 g ai/ha | | | | | | | | | | | | | | | | | |
| | Compounds | | Compounds | | | | | | | Compounds | | | | | | | | | | | | | | | | | |
| | 444 | 445 | 54 | 72 | 73 | 74 | 152 | 191 | 192 | 1 | 2 | 3 | 4 | 5 | 6 | 7 | 8 | 9 | 10 | 11 | 12 | 13 | 14 | 15 | 16 | 17 | 18 |
| Barnyardgrass | 0 | 100 | 10 | 0 | 10 | 0 | 90 | 10 | 70 | 80 | 90 | 90 | 70 | 30 | 100 | 60 | 0 | 90 | 100 | 80 | 10 | 80 | 100 | 30 | 100 | 80 | 50 |
| Cocklebur | 0 | 90 | 0 | — | 0 | 10 | 10 | 0 | 0 | 30 | — | 20 | 0 | 0 | 90 | 0 | 0 | 40 | 60 | 0 | 0 | 0 | — | — | 100 | 100 | — |
| Corn | 0 | 80 | 0 | 0 | 20 | 0 | 50 | 0 | 40 | 50 | 80 | 20 | 0 | 0 | 70 | 0 | 0 | 20 | 0 | 0 | 0 | 0 | 30 | 0 | 50 | 30 | 0 |
| Crabgrass, Large | 0 | 100 | 40 | 0 | 10 | 0 | 100 | 40 | 90 | 100 | 100 | 100 | 80 | 70 | 100 | 90 | 0 | 70 | 80 | 100 | 0 | 90 | 100 | 70 | 100 | 60 | 100 |
| Foxtail, Giant | 0 | 100 | 10 | 0 | 20 | 10 | 100 | 10 | 60 | 100 | 100 | 100 | 60 | 20 | 100 | 80 | 0 | 80 | 70 | 90 | 10 | 60 | 100 | 10 | 90 | 70 | 20 |
| Lambsquarters | 0 | 100 | 0 | 0 | 10 | 0 | 100 | 80 | 100 | 100 | 100 | 90 | 100 | 60 | 100 | 100 | 0 | 100 | 100 | 100 | 0 | 90 | 100 | 70 | 100 | 100 | 80 |
| Morningglory | 0 | 100 | 10 | 0 | 10 | 10 | 60 | 10 | 20 | 60 | 30 | 20 | 0 | 0 | 40 | 10 | 0 | — | 10 | 0 | 0 | 20 | 20 | 0 | 0 | 0 | 100 |
| Pigweed | 0 | 100 | 0 | 0 | 0 | 0 | 100 | 90 | 100 | 100 | 100 | 100 | 100 | 90 | 100 | 100 | 0 | 100 | 100 | 90 | 0 | 100 | 100 | 90 | 100 | 80 | 80 |
| Surinam Grass | 0 | 100 | 0 | 0 | 20 | 0 | 60 | 0 | 30 | 70 | 70 | 40 | 30 | 0 | 90 | 70 | 0 | 70 | 60 | 50 | 0 | 50 | 90 | 10 | 70 | 40 | 30 |
| Velvetleaf | 0 | 100 | 0 | 0 | 10 | 0 | 90 | 20 | 50 | 70 | 90 | 80 | 50 | 20 | 100 | 30 | 0 | 70 | 50 | 50 | 0 | 70 | 100 | 10 | 90 | 60 | 30 |
| Wheat | 0 | 100 | 0 | 0 | 20 | 0 | 100 | 0 | 10 | 60 | 100 | 90 | 90 | 50 | 100 | 60 | 0 | 70 | 100 | 10 | 0 | 50 | 90 | 20 | 100 | 30 | 40 |
| | 125 g ai/ha | | | | | | | | | | | | | | | | | | | | | | | | | | |
| | Compounds | | | | | | | | | | | | | | | | | | | | | | | | | | |
| | 19 | 20 | 21 | 22 | 23 | 24 | 25 | 26 | 27 | 28 | 29 | 30 | 31 | 32 | 33 | 34 | 35 | 36 | 37 | 40 | 41 | 43 | 44 | 45 | 46 | 47 | 48 |
| Barnyardgrass | 10 | 10 | 10 | 0 | 0 | 0 | 0 | 0 | 100 | 80 | 70 | 80 | 80 | 20 | 60 | 50 | 40 | 30 | 10 | 0 | 40 | 100 | 100 | 100 | 60 | 70 | 70 |
| Cocklebur | 0 | 0 | — | 0 | 0 | 0 | 0 | 0 | 0 | — | — | 0 | 10 | 0 | 20 | 0 | 20 | 0 | 0 | 0 | 0 | 0 | 0 | 0 | 20 | 0 | 10 |
| Corn | 0 | 0 | 0 | 0 | 0 | 0 | 0 | 0 | 60 | 0 | 0 | 10 | 0 | 0 | 0 | 0 | 0 | 0 | 0 | 0 | 0 | 0 | 0 | 0 | 10 | 0 | 0 |
| Crabgrass, Large | 0 | 20 | 20 | 0 | 0 | 10 | 0 | 0 | 100 | — | 100 | 20 | 10 | 30 | 90 | 80 | 70 | 10 | 30 | 0 | 70 | 40 | 0 | 40 | 90 | 80 | 20 |
| Foxtail, Giant | 0 | 0 | 0 | 0 | 0 | 0 | 0 | 0 | 100 | 10 | 80 | 30 | 60 | 0 | 80 | 30 | 80 | 0 | 20 | 0 | 10 | 10 | 0 | 10 | 60 | 40 | 70 |
| Lambsquarters | 10 | 20 | 0 | 0 | 0 | 0 | 0 | 0 | 100 | 90 | 90 | 90 | 90 | 100 | 100 | 80 | 90 | 80 | 10 | 40 | 80 | 90 | 80 | 100 | 100 | 100 | 90 |
| Morningglory | 0 | 10 | 10 | 0 | 0 | 0 | 0 | 0 | — | 20 | 10 | 0 | — | 50 | 10 | 0 | 10 | 10 | 0 | — | 0 | 0 | 0 | 10 | 10 | 20 | 10 |
| Pigweed | 0 | 30 | 0 | 0 | 0 | 0 | 0 | 0 | 100 | 90 | 100 | 80 | 70 | 0 | 100 | 100 | 100 | 90 | 80 | 30 | 70 | 100 | 100 | 100 | 100 | 100 | 100 |
| Rice | 0 | 10 | 10 | 0 | 0 | 0 | 0 | 0 | — | — | | | | | | | | | | | | | | | — | — | 10 |
| Surinam Grass | 0 | 10 | 0 | 0 | 0 | 0 | 0 | 0 | 60 | 30 | 60 | 0 | 30 | 0 | 30 | 20 | 40 | 10 | 10 | 0 | 10 | 30 | 0 | 20 | 20 | 20 | 10 |
| Velvetleaf | 20 | 0 | 0 | 0 | 0 | 0 | 0 | 0 | 100 | 60 | 50 | 10 | 80 | 0 | 40 | 20 | 50 | 20 | 20 | 0 | 0 | 30 | 20 | 20 | 50 | 30 | 90 |
| | 125 g ai/ha | | | | | | | | | | | | | | | | | | | | | | | | | | |
| | Compounds | | | | | | | | | | | | | | | | | | | | | | | | | | |
| | 49 | 50 | 51 | 52 | 55 | 56 | 57 | 58 | 59 | 60 | 61 | 62 | 63 | 64 | 65 | 66 | 67 | 68 | 69 | 70 | 71 | 75 | 76 | 77 | 78 | 79 | 80 |
| Barnyardgrass | 30 | 40 | 80 | 10 | 80 | 50 | 50 | 90 | 10 | 50 | 20 | 20 | 60 | 50 | 50 | 0 | 0 | 0 | 0 | 40 | 0 | 50 | 80 | 0 | 0 | 0 | 0 |
| Cocklebur | 0 | 0 | 0 | 0 | 0 | 0 | 0 | 0 | 0 | 0 | 0 | 0 | 0 | 0 | 0 | 0 | 0 | 0 | 0 | 0 | 0 | 0 | — | 0 | 0 | 0 | 0 |
| Corn | 0 | 0 | 0 | 0 | 0 | 0 | 0 | 20 | 0 | 0 | 0 | 0 | 0 | 0 | 0 | 0 | 0 | 0 | 0 | 10 | 0 | 0 | 30 | 0 | 0 | 0 | 0 |
| Crabgrass, Large | 20 | 30 | 90 | 20 | 90 | 70 | 40 | 100 | 10 | 90 | 50 | 80 | 70 | 20 | 20 | 0 | 0 | 0 | 0 | 100 | 0 | 10 | 80 | 0 | 0 | 0 | 0 |
| Foxtail, Giant | 0 | 20 | 100 | 10 | 60 | 60 | 20 | 100 | 0 | 40 | 20 | 70 | 60 | 10 | 20 | 0 | 0 | 0 | 0 | 90 | 0 | 30 | 90 | 0 | 10 | 10 | 20 |
| Lambsquarters | 70 | 90 | 100 | 0 | 70 | 100 | 100 | 100 | 90 | 90 | 100 | 100 | 90 | 60 | 80 | 0 | 0 | 20 | 0 | — | 0 | 20 | 50 | 60 | 0 | 0 | 0 |
| Morningglory | 0 | 10 | 0 | 0 | 0 | 20 | 0 | — | 0 | 10 | — | 10 | 0 | 0 | 0 | 0 | 0 | 20 | 0 | 10 | 0 | 0 | 10 | 0 | 0 | 0 | 0 |
| Pigweed | 90 | 100 | 100 | 0 | 80 | 30 | 90 | 100 | 100 | 90 | 80 | 100 | 100 | 100 | 90 | 0 | 0 | 70 | 0 | 30 | 0 | 50 | 100 | 90 | 0 | 0 | 0 |
| Rice | 40 | 10 | 0 | 0 | 50 | 40 | 30 | 60 | 0 | 30 | 0 | 0 | 50 | 0 | 10 | 0 | 0 | 0 | 0 | 0 | 0 | 40 | 50 | 0 | — | — | — |
| Surinam Grass | 10 | 20 | 30 | 0 | 50 | 30 | 20 | 30 | 0 | 10 | 10 | 10 | 20 | 10 | 10 | 0 | 0 | 0 | 0 | 40 | 0 | 30 | 70 | 0 | 0 | 0 | 0 |
| Velvetleaf | 0 | 20 | 50 | 0 | 0 | 0 | 0 | 60 | 10 | 10 | 70 | 80 | 10 | 0 | 10 | 0 | 0 | 0 | 0 | 30 | 0 | 20 | 30 | 0 | 10 | 10 | 20 |
| | 125 g ai/ha | | | | | | | | | | | | | | | | | | | | | | | | | | |
| | Compounds | | | | | | | | | | | | | | | | | | | | | | | | | | |
| | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 104 | 105 | 106 | 107 | 108 |
| Barnyardgrass | 10 | 0 | 0 | 0 | 0 | 0 | 20 | 70 | 0 | 10 | 0 | 0 | 0 | 10 | 50 | 70 | 0 | 0 | 0 | 0 | 40 | 80 | 0 | 0 | — | 0 | 80 |
| Cocklebur | 0 | 0 | — | 0 | 0 | 0 | 0 | 0 | 0 | 0 | — | 0 | 0 | 0 | 0 | 0 | 0 | 0 | 0 | 0 | 0 | 0 | 0 | 0 | 0 | 0 | 10 |
| Corn | 0 | 0 | 0 | 0 | 0 | 0 | 0 | 0 | 0 | 0 | 0 | 0 | 10 | 0 | 60 | 40 | 0 | 0 | 0 | 0 | 20 | 0 | 0 | 0 | 0 | 0 | 10 |
| Crabgrass, Large | 0 | 0 | 0 | 0 | 0 | 0 | 90 | 90 | 0 | 10 | — | 0 | 10 | 10 | 60 | 20 | 0 | 0 | 20 | 0 | 50 | 100 | 80 | 0 | 90 | 0 | 100 |
| Foxtail, Giant | 10 | 0 | 0 | 0 | 0 | 0 | 10 | 70 | 0 | 10 | 10 | 0 | 10 | 10 | 50 | 60 | 0 | 0 | 10 | 0 | 20 | 30 | 0 | 0 | 10 | 0 | 90 |
| Lambsquarters | 0 | 0 | 0 | 0 | 0 | 0 | 70 | 100 | 0 | 0 | 0 | 0 | 0 | 0 | 100 | 100 | 0 | 0 | 0 | 0 | 100 | 100 | 60 | 0 | 0 | 0 | 100 |
| Morningglory | 10 | 0 | 0 | 0 | 0 | 0 | 0 | 0 | 0 | 0 | 0 | 0 | 0 | 0 | 10 | 0 | 0 | 0 | 0 | 0 | 10 | 50 | 0 | 0 | — | 0 | 50 |
| Pigweed | 20 | 0 | 0 | 0 | 0 | 0 | 40 | 100 | 0 | 10 | 0 | 0 | 0 | 0 | 100 | 100 | 0 | 0 | 0 | 0 | 100 | 90 | 60 | 0 | 0 | 0 | 90 |
| Rice | 0 | 0 | 0 | 0 | 0 | 0 | 0 | 20 | 0 | 0 | 0 | 0 | 0 | 0 | 30 | 60 | 0 | 0 | 0 | 0 | 30 | — | — | — | — | — | |
| Surinam Grass | 0 | 0 | 0 | 0 | 0 | 0 | 0 | 20 | 0 | 0 | — | 0 | 0 | 0 | 20 | 20 | 0 | 0 | 10 | 0 | 10 | 20 | 0 | 0 | 0 | 0 | 50 |
| Velvetleaf | 0 | 0 | 0 | 0 | 0 | 0 | 0 | 40 | 0 | 0 | — | 0 | 0 | 0 | 0 | 50 | 0 | 0 | 10 | 0 | 20 | 0 | 0 | 0 | 0 | 0 | 80 |
| | 125 g ai/ha | | | | | | | | | | | | | | | | | | | | | | | | | | |
| | Compounds | | | | | | | | | | | | | | | | | | | | | | | | | | |
| | 109 | 110 | 112 | 113 | 114 | 115 | 117 | 118 | 119 | 120 | 121 | 122 | 123 | 124 | 125 | 126 | 127 | 128 | 130 | 131 | 132 | 133 | 134 | 135 | 136 | 137 | 138 |
| Barnyardgrass | 80 | 10 | 50 | 60 | 50 | 80 | 60 | 10 | 0 | 0 | 0 | 80 | 0 | 0 | 0 | 40 | 20 | 80 | 10 | 70 | 10 | 10 | 50 | 40 | 90 | 10 | 10 |
| Cocklebur | 0 | 0 | 0 | 0 | 10 | 20 | 20 | 0 | 0 | 0 | 0 | 20 | 0 | 0 | 0 | 0 | 10 | 20 | 0 | 0 | 0 | 0 | 0 | 0 | 0 | 0 | 0 |
| Corn | 0 | 0 | 0 | 0 | 0 | 0 | 0 | 0 | 0 | 0 | 0 | 0 | 0 | 0 | 0 | 0 | 0 | 0 | 10 | 0 | 10 | 0 | 0 | 0 | 10 | 10 | 0 |
| Crabgrass, Large | 80 | 80 | 20 | 100 | 90 | 90 | 90 | 90 | 20 | 70 | 0 | 90 | 0 | 0 | 0 | 0 | 90 | 90 | 90 | 50 | 90 | 20 | 80 | 50 | 100 | 80 | 40 |
| Foxtail, Giant | 70 | 10 | 10 | 10 | 50 | 80 | 20 | 10 | 0 | 0 | 0 | 50 | 0 | 0 | 0 | 0 | 30 | 40 | 20 | 10 | 30 | 0 | 20 | 10 | 80 | 40 | 30 |
| Lambsquarters | 20 | 20 | 0 | 80 | 100 | 100 | 90 | 0 | 0 | 0 | 70 | 80 | 0 | 0 | 0 | 0 | 0 | 0 | 0 | 100 | 60 | 50 | 100 | 30 | 100 | 10 | 0 |
| Morningglory | 10 | 20 | 0 | 0 | 10 | 30 | — | 10 | 0 | 10 | 0 | 20 | 0 | 0 | 0 | 0 | 0 | 20 | 10 | 10 | 10 | 10 | 10 | 10 | 50 | 0 | 0 |
| Pigweed | 30 | 0 | 0 | 0 | 80 | 80 | 60 | 60 | 0 | 0 | 80 | 70 | 0 | 30 | 0 | 0 | 40 | 50 | 20 | 100 | 100 | 100 | 100 | 60 | 100 | 80 | 60 |
| Rice | — | — | — | — | — | 0 | 20 | 0 | 0 | 0 | 0 | 20 | 0 | 0 | | | | | | | | | | | | | |
| Surinam Grass | 60 | 10 | 20 | 10 | 20 | 70 | 30 | 10 | 0 | 0 | 0 | 20 | 0 | 0 | 0 | 0 | 50 | 40 | 60 | 10 | 40 | 0 | 30 | 10 | 40 | 20 | 0 |
| Velvetleaf | 20 | 0 | 0 | 20 | 40 | 80 | 60 | 0 | 0 | 20 | 0 | 40 | 0 | 0 | 0 | 0 | 10 | 70 | 10 | 50 | 30 | 20 | 30 | 30 | 50 | 10 | 0 |
| | 125 g ai/ha | | | | | | | | | | | | | | | | | | | | | | | | | | |
| | Compounds | | | | | | | | | | | | | | | | | | | | | | | | | | |
| | 139 | 140 | 141 | 142 | 143 | 144 | 145 | 146 | 147 | 148 | 149 | 150 | 151 | 153 | 154 | 155 | 156 | 157 | 158 | 159 | 160 | 161 | 162 | 163 | 164 | 165 | 166 |
| Barnyardgrass | 80 | 20 | 0 | 70 | 0 | 90 | 70 | 40 | 70 | 0 | 80 | 30 | 0 | 60 | 10 | 60 | 100 | 0 | 70 | 70 | 70 | 50 | 100 | 60 | 0 | 0 | 0 |
| Cocklebur | 0 | — | 0 | 30 | 0 | 10 | 0 | 0 | 0 | 0 | 0 | 0 | 0 | 0 | 0 | 0 | 100 | 0 | 40 | 90 | 40 | 0 | 30 | 70 | 0 | 0 | 0 |
| Corn | 10 | 0 | 0 | 0 | 0 | 10 | 0 | 0 | 10 | 0 | 10 | 0 | 0 | 0 | 0 | 0 | 40 | 0 | 0 | 0 | 0 | 0 | 30 | 0 | 0 | 0 | 0 |
| Crabgrass, Large | 60 | 0 | 10 | 80 | 0 | 80 | — | 10 | 90 | 0 | 90 | 0 | 0 | 50 | 50 | 60 | 90 | 0 | 80 | 40 | 20 | 80 | 100 | 60 | 0 | 0 | 0 |
| Foxtail, Giant | 70 | 0 | 10 | 60 | 0 | 100 | 80 | 10 | 70 | 0 | 60 | 0 | 0 | 0 | 0 | 20 | 100 | 0 | 50 | 50 | 10 | 20 | 100 | 20 | 0 | 0 | 0 |
| Lambsquarters | 100 | 0 | 0 | 100 | 0 | 100 | 80 | 80 | 90 | 0 | 60 | 0 | 0 | 100 | 70 | 100 | 100 | 0 | 70 | 100 | 100 | 100 | 100 | 80 | 0 | 0 | 0 |
| Morningglory | 10 | — | 20 | — | 0 | 100 | 30 | 0 | 0 | 0 | 20 | 0 | 0 | 0 | 0 | 20 | 60 | 0 | 50 | 30 | 20 | 10 | 20 | 60 | 0 | 0 | 0 |
| Pigweed | 90 | 0 | 0 | 100 | 0 | 100 | 100 | 100 | 100 | 0 | 100 | 70 | 0 | 100 | 90 | 100 | 100 | 0 | 100 | 100 | 100 | 100 | 100 | 90 | 0 | 0 | 0 |
| Rice | 30 | 0 | 0 | 30 | — | 20 | 0 | 10 | 0 | 0 | 20 | 0 | 0 | 10 | 0 | 20 | 90 | 0 | 80 | — | — | 0 | 90 | 80 | — | 0 | 0 |
| Surinam Grass | 30 | 0 | 0 | 10 | 0 | 60 | 30 | 10 | 20 | 0 | 40 | 10 | 0 | 10 | 0 | 10 | 90 | 0 | 30 | 60 | 20 | 10 | 90 | 50 | 0 | 0 | 0 |
| Velvetleaf | 50 | 0 | 0 | 80 | 0 | 100 | 80 | 20 | 30 | 0 | 0 | 0 | 0 | 40 | 70 | 20 | 100 | 0 | 50 | 80 | 60 | 20 | 90 | 70 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 167 | 168 | 169 | 170 | 171 | 179 | 180 | 181 | 182 | 183 | 184 | 185 | 186 | 187 | 188 | 189 | 190 | 193 | 194 | 195 | 196 | 197 | 198 | 199 | 200 | 201 | 202 |
| Barnyardgrass | 0 | 70 | 0 | 0 | 0 | 0 | 0 | 30 | 20 | 20 | 0 | 0 | 0 | 70 | 80 | 0 | 0 | 90 | 0 | 40 | 0 | 0 | 40 | 20 | 0 | 0 | 20 |
| Cocklebur | — | 0 | 0 | 0 | 0 | 0 | 0 | 10 | 0 | 0 | 0 | 0 | 0 | 0 | 0 | 0 | 0 | 10 | 0 | 0 | 0 | 0 | 0 | 0 | 0 | 0 | 0 |
| Corn | — | 0 | 0 | 0 | 0 | 0 | 0 | 0 | 0 | 0 | 0 | 0 | 0 | 20 | 10 | 0 | 0 | 70 | 0 | 0 | 0 | 0 | 0 | 0 | 0 | 0 | 0 |
| Crabgrass, Large | 0 | 100 | 0 | 20 | 0 | 0 | 0 | 50 | 70 | 10 | 30 | 0 | 0 | 70 | 100 | 30 | 30 | 100 | 100 | 80 | 0 | 0 | 80 | 90 | 50 | 0 | 60 |
| Foxtail, Giant | 0 | 90 | 0 | 0 | — | 0 | 0 | 20 | 30 | 0 | 0 | 0 | 0 | 10 | 50 | 0 | 0 | 100 | 10 | 20 | 0 | 0 | 20 | 10 | 0 | 0 | 10 |
| Lambsquarters | 0 | 90 | 0 | 0 | 0 | 0 | 0 | 50 | 20 | 50 | 50 | 20 | 0 | 20 | 90 | 0 | 0 | 100 | 30 | 0 | 0 | 0 | 100 | 80 | 20 | 0 | 30 |
| Morningglory | 0 | 0 | 0 | 0 | 0 | 0 | 0 | 0 | 10 | 10 | 10 | 0 | 0 | 20 | 10 | — | 0 | 40 | 20 | 10 | 0 | 0 | 10 | 0 | 0 | 0 | 0 |
| Pigweed | 0 | 100 | 0 | 0 | 0 | 0 | 0 | 30 | 100 | 100 | 100 | 0 | 0 | 20 | 40 | 90 | 30 | 100 | 0 | 90 | 0 | 0 | 90 | 90 | 10 | 0 | 10 |
| Rice | 0 | 0 | 0 | 0 | 0 | — | — | 40 | 0 | 0 | 0 | 0 | 0 | 30 | 50 | 0 | 0 | 80 | 0 | 10 | — | 0 | 30 | 0 | 0 | 0 | 10 |
| Surinam Grass | 0 | 50 | 0 | 0 | — | 0 | 0 | 30 | 20 | 20 | 0 | 10 | 0 | 30 | 30 | 0 | 0 | 80 | 0 | 10 | 0 | 0 | 10 | 10 | 0 | 0 | 0 |
| Velvetleaf | 0 | 60 | 0 | 0 | 0 | 0 | 0 | 0 | 0 | 10 | 10 | 10 | 10 | 10 | 70 | 0 | 0 | 70 | 20 | 10 | 0 | 0 | 0 | 0 | 0 | 0 | 10 |
| | 125 g ai/ha | | | | | | | | | | | | | | | | | | | | | | | | | | |
| | Compounds | | | | | | | | | | | | | | | | | | | | | | | | | | |
| | 203 | 204 | 205 | 206 | 207 | 209 | 210 | 211 | 212 | 213 | 214 | 215 | 216 | 217 | 218 | 219 | 220 | 221 | 222 | 223 | 224 | 225 | 226 | 227 | 228 | 229 | 230 |
| Barnyardgrass | 0 | 0 | 50 | 10 | 80 | 50 | 70 | 10 | 80 | 10 | 0 | 10 | 0 | 0 | 0 | 0 | 0 | 0 | 90 | 20 | 30 | 60 | 10 | 0 | 80 | 50 | 10 |
| Cocklebur | 0 | 0 | 10 | 0 | 10 | 10 | 10 | 0 | 0 | 0 | 0 | 0 | 0 | 0 | 0 | 0 | 0 | 0 | 30 | 0 | 20 | 0 | 0 | 0 | 30 | 0 | 0 |
| Corn | 0 | 0 | 10 | 0 | 10 | 0 | 20 | 0 | 10 | 0 | 0 | 0 | 0 | 0 | 0 | 0 | 0 | 0 | 60 | 10 | 20 | 30 | 0 | 0 | 60 | 10 | 0 |
| Crabgrass, Large | 30 | 0 | 60 | 10 | 70 | 40 | 90 | 10 | 60 | 10 | 0 | 0 | 0 | 0 | 0 | 0 | 20 | 80 | 90 | 50 | 30 | 10 | 20 | 40 | 60 | 40 | 20 |
| Foxtail, Giant | 0 | 0 | 50 | 0 | 50 | 30 | 80 | 10 | 70 | 20 | 0 | 0 | 0 | 0 | 0 | 0 | 0 | 0 | 60 | 10 | 10 | 60 | 20 | 10 | 70 | 50 | 10 |
| Lambsquarters | 10 | 0 | 80 | 0 | 80 | 60 | 100 | 10 | 100 | 30 | 0 | 90 | 90 | 0 | 0 | 0 | 0 | 20 | 100 | 80 | 100 | 80 | — | 0 | 100 | 90 | 20 |
| Morningglory | 0 | — | 0 | 0 | 20 | 10 | 20 | 0 | 0 | 0 | 0 | 10 | 0 | 0 | 0 | 0 | 0 | 0 | — | 10 | — | 0 | 0 | 0 | 10 | 10 | 10 |
| Pigweed | 10 | 0 | 100 | 0 | 90 | 70 | 100 | 0 | 100 | 30 | 0 | 80 | 0 | 0 | 0 | 0 | 0 | 50 | 90 | 90 | 80 | 70 | 40 | 0 | 90 | 100 | 40 |
| Rice | 0 | 0 | 10 | 0 | 50 | 30 | 60 | 0 | 10 | 0 | 0 | 0 | 0 | 0 | 0 | 0 | 0 | 0 | 90 | 0 | 30 | 60 | 20 | 10 | 80 | 30 | 0 |
| Surinam Grass | 0 | 0 | 20 | 0 | 40 | 20 | 60 | 0 | 50 | 0 | 0 | 0 | 0 | 0 | 0 | 0 | 0 | 0 | 90 | 20 | 60 | 20 | 10 | 30 | 60 | 40 | 0 |
| Velvetleaf | 0 | 0 | 30 | 0 | 20 | 10 | 10 | 0 | 10 | 0 | 0 | 0 | 0 | 0 | 0 | 0 | 0 | 0 | 100 | 30 | 70 | 30 | 10 | 30 | 100 | 40 | 10 |
| | 125 g ai/ha | | | | | | | | | | | | | | | | | | | | | | | | | | |
| | Compounds | | | | | | | | | | | | | | | | | | | | | | | | | | |
| | 231 | 232 | 233 | 234 | 235 | 236 | 237 | 238 | 239 | 240 | 241 | 242 | 243 | 244 | 245 | 246 | 247 | 248 | 249 | 250 | 252 | 253 | 254 | 256 | 257 | 258 | 259 |
| Barnyardgrass | 50 | 20 | 10 | 0 | 10 | 50 | 20 | 0 | 0 | 0 | 0 | 80 | 70 | 80 | 70 | 60 | 30 | 0 | 0 | 0 | 0 | 0 | 0 | 0 | 0 | 70 | 0 |
| Cocklebur | 0 | 0 | 0 | 0 | 0 | 0 | 0 | 0 | 0 | 0 | 0 | 10 | 0 | 0 | 0 | 0 | 0 | 0 | 0 | 0 | 0 | 0 | 0 | 0 | 0 | 0 | 0 |
| Corn | 0 | 0 | 0 | 0 | 0 | 0 | 0 | 0 | 0 | 0 | 0 | 30 | 20 | 30 | 0 | 0 | 0 | 0 | 0 | 0 | 0 | 0 | 0 | 0 | 0 | 0 | 0 |
| Crabgrass, Large | 30 | 60 | 20 | 0 | 40 | 30 | 30 | 0 | 0 | 20 | 90 | 90 | 60 | 90 | 60 | 70 | 20 | 20 | 0 | 0 | 80 | 20 | 10 | 10 | 0 | 10 | 0 |
| Foxtail, Giant | 60 | 0 | 0 | 0 | 0 | 10 | 0 | 0 | 0 | 0 | 10 | 80 | 50 | 80 | 60 | 30 | 10 | 20 | 0 | 0 | 10 | 0 | 0 | 0 | 0 | 0 | 0 |
| Lambsquarters | 80 | 80 | 0 | 0 | 50 | 50 | 70 | 0 | 20 | 0 | 80 | 100 | 90 | 90 | 90 | 70 | 80 | 0 | 0 | 0 | — | 0 | — | — | 0 | — | 0 |
| Morningglory | 10 | 10 | 0 | 0 | 10 | 0 | 0 | 0 | 0 | 0 | 0 | 0 | 0 | 0 | 10 | 0 | 0 | 0 | 0 | 0 | 0 | 0 | 0 | 0 | 0 | 0 | 0 |
| Pigweed | 60 | 90 | 0 | 0 | 30 | 30 | 20 | 0 | 0 | 0 | 100 | 100 | 60 | 90 | 100 | 100 | 100 | 0 | 0 | 0 | — | — | 0 | — | 0 | 0 | 0 |
| Rice | 20 | 10 | 0 | 0 | 0 | 40 | 0 | 0 | 0 | 0 | 0 | 50 | 20 | 60 | 20 | 40 | 10 | 0 | 0 | 0 | 30 | 0 | 0 | 30 | 0 | 20 | 0 |
| Surinam Grass | 20 | 0 | 0 | 0 | 10 | 20 | 10 | 0 | 0 | 0 | 10 | 70 | 60 | 70 | 40 | 30 | 20 | 20 | 0 | 10 | 10 | 10 | 0 | 60 | 0 | 0 | 0 |
| Velvetleaf | 30 | 20 | 10 | 0 | 10 | 20 | 30 | 0 | 0 | 0 | 20 | 50 | 40 | 70 | 40 | 60 | 20 | 0 | 0 | 0 | 0 | 0 | 0 | 0 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 260 | 261 | 262 | 263 | 264 | 265 | 266 | 267 | 268 | 269 | 270 | 271 | 272 | 273 | 274 | 275 | 276 | 277 | 278 | 279 | 280 | 281 | 282 | 283 | 284 | 285 | 286 |
| Barnyardgrass | 0 | 0 | 0 | 10 | 0 | 0 | 60 | 20 | 100 | 80 | 0 | 0 | 10 | 30 | 60 | 0 | 60 | 0 | 30 | 20 | 0 | 20 | 30 | 0 | 0 | 30 | 30 |
| Cocklebur | 0 | 0 | 0 | — | 0 | 0 | 0 | 0 | 80 | 20 | 0 | 0 | 0 | 0 | 0 | 0 | 0 | 0 | 0 | 0 | 0 | 0 | 0 | 0 | 0 | 0 | 0 |
| Corn | 0 | — | 0 | 0 | 0 | 0 | 0 | 0 | 80 | 60 | 0 | 0 | 0 | 0 | 0 | 0 | 20 | 0 | 0 | 0 | 0 | 0 | 0 | — | 0 | 0 | 0 |
| Crabgrass, Large | 0 | 50 | 0 | 10 | 10 | — | 50 | 40 | 100 | 100 | 0 | 0 | 80 | 80 | 10 | 0 | 10 | 0 | 20 | 10 | 0 | 10 | 10 | 0 | 0 | 20 | 0 |
| Foxtail, Giant | 0 | 0 | 0 | 0 | 0 | 0 | 80 | 30 | 100 | 80 | 0 | 0 | 30 | 50 | 10 | 0 | 30 | 0 | 20 | 10 | 0 | 10 | 10 | 0 | 0 | 10 | 10 |
| Lambsquarters | — | — | 40 | 70 | 0 | — | 100 | 0 | 100 | 100 | — | 0 | 100 | 70 | 70 | — | 40 | 40 | 100 | 100 | — | 90 | 100 | 0 | 90 | 100 | 100 |
| Morningglory | 0 | 0 | 0 | 0 | 0 | 0 | 0 | 20 | 0 | 10 | 0 | 0 | 0 | 0 | 0 | 0 | 0 | 0 | 0 | 0 | 0 | 0 | 0 | 0 | 0 | 0 | 0 |
| Pigweed | — | 0 | 50 | 80 | 0 | 0 | 100 | — | 100 | 100 | 0 | 0 | 90 | 70 | 30 | 0 | 30 | 60 | 100 | 90 | 70 | 100 | 100 | 0 | 90 | 100 | 100 |
| Rice | 0 | 0 | 0 | 0 | 0 | 0 | 70 | 20 | 90 | 70 | 0 | 0 | 0 | 0 | 30 | 0 | 50 | 0 | 30 | 20 | 0 | 20 | 10 | 0 | 0 | 40 | 30 |
| Surinam Grass | 0 | 0 | 0 | 10 | 0 | 0 | 60 | 10 | 80 | 50 | 0 | 0 | 10 | 20 | 50 | 0 | 10 | 0 | 20 | 0 | 0 | 10 | 0 | 0 | 0 | 10 | 10 |
| Velvetleaf | 0 | 0 | 0 | 10 | 0 | 0 | 20 | 20 | 100 | 60 | 50 | 0 | 10 | 10 | 20 | 0 | 0 | 0 | 0 | 0 | 10 | 10 | 10 | 0 | 0 | 30 | 0 |
| | 125 g ai/ha | | | | | | | | | | | | | | | | | | | | | | | | | | |
| | Compounds | | | | | | | | | | | | | | | | | | | | | | | | | | |
| | 287 | 288 | 289 | 290 | 291 | 292 | 293 | 294 | 295 | 296 | 297 | 298 | 299 | 300 | 301 | 302 | 303 | 304 | 305 | 306 | 307 | 308 | 309 | 310 | 311 | 312 | 313 |
| Barnyardgrass | 80 | 0 | 0 | 0 | 0 | 10 | 90 | 100 | 10 | 30 | 0 | 70 | 90 | 70 | 60 | 0 | 70 | 10 | 80 | 60 | 80 | 20 | 80 | 60 | 0 | 0 | 80 |
| Cocklebur | 0 | 0 | 0 | 0 | 0 | 0 | 60 | 90 | 0 | 0 | 0 | 0 | 20 | 10 | 0 | 0 | 0 | 0 | 50 | 0 | 80 | 0 | 30 | 10 | 0 | 0 | 0 |
| Corn | 0 | 0 | 0 | 0 | 0 | 0 | 70 | 80 | 0 | 30 | 0 | 30 | 50 | 0 | 0 | 0 | — | 0 | 10 | 70 | 70 | 20 | 60 | 40 | 0 | 0 | 10 |
| Crabgrass, Large | 70 | 0 | 0 | 0 | 0 | 80 | 100 | 100 | 10 | 40 | 20 | 100 | 100 | 100 | 100 | 70 | 100 | 10 | 90 | 100 | 90 | 40 | 60 | 90 | 0 | 0 | 30 |
| Foxtail, Giant | 10 | 0 | 0 | 10 | 0 | 0 | 60 | 90 | 10 | 70 | 10 | 80 | 100 | 80 | 70 | 0 | 50 | 10 | 80 | 40 | 90 | 10 | 80 | 50 | 0 | 0 | 40 |
| Lambsquarters | 100 | 0 | 0 | 100 | 0 | 70 | 80 | 100 | 0 | 90 | 0 | 100 | 100 | 100 | 100 | 0 | 100 | 100 | 100 | 100 | 100 | 100 | 90 | 100 | 0 | 0 | 100 |
| Morningglory | 0 | 0 | 0 | 0 | 0 | 0 | 40 | 0 | 0 | 10 | 10 | 10 | 40 | 10 | 10 | 0 | 10 | 10 | 0 | 10 | 0 | 0 | 0 | 0 | 0 | 0 | 10 |
| Pigweed | 90 | 0 | 0 | 0 | 0 | 40 | 10 | 100 | 0 | 70 | 0 | 100 | 100 | 100 | 100 | 100 | 100 | 100 | 100 | 100 | 100 | 80 | 70 | 80 | 0 | 0 | 100 |
| Rice | 80 | 0 | 0 | 0 | 0 | 0 | 90 | 90 | 10 | — | 0 | 40 | 80 | 40 | 10 | 0 | 70 | 0 | 70 | 60 | 80 | 20 | 70 | 40 | — | — | 20 |
| Surinam Grass | 50 | 0 | 0 | 0 | 0 | 20 | 90 | 100 | 0 | 20 | 10 | 80 | 100 | 80 | 90 | 10 | 50 | 10 | 80 | 30 | 70 | 20 | 60 | 30 | 0 | 0 | 30 |
| Velvetleaf | 70 | 0 | 0 | 0 | 0 | 30 | 100 | 100 | 50 | 70 | 0 | 10 | 100 | 90 | 100 | 0 | 10 | 90 | 80 | 30 | 100 | 50 | 80 | 60 | 0 | 0 | 30 |
| | 125 g ai/ha | | | | | | | | | | | | | | | | | | | | | | | | | | |
| | Compounds | | | | | | | | | | | | | | | | | | | | | | | | | | |
| | 314 | 315 | 316 | 317 | 318 | 319 | 329 | 330 | 331 | 332 | 333 | 334 | 335 | 336 | 338 | 340 | 341 | 342 | 343 | 344 | 345 | 346 | 347 | 348 | 349 | 350 | 351 |
| Barnyardgrass | 10 | 0 | 0 | 0 | 40 | 50 | 0 | 0 | 60 | 10 | 0 | 90 | 50 | 100 | 20 | 80 | 80 | 70 | 70 | 20 | 20 | 0 | 0 | 0 | 80 | 0 | 0 |
| Cocklebur | 0 | 0 | 0 | 0 | 0 | 0 | 0 | 0 | 0 | 0 | 0 | 60 | 0 | 10 | 0 | 20 | 40 | 40 | — | 20 | 0 | 0 | 0 | 0 | 0 | 0 | 0 |
| Corn | 0 | 0 | 0 | 0 | 0 | 10 | 0 | 0 | 20 | 0 | 0 | 20 | 30 | 50 | 10 | — | — | — | — | 10 | 0 | 0 | 0 | 0 | 70 | 0 | 0 |
| Crabgrass, Large | 20 | 0 | 0 | 0 | 10 | 50 | 0 | 0 | 60 | 40 | 40 | 90 | 50 | 90 | 80 | 80 | 90 | 40 | 40 | 60 | 30 | 60 | 0 | 0 | 100 | 0 | 0 |
| Foxtail, Giant | 30 | 0 | 0 | 0 | 0 | 20 | 0 | 0 | 20 | 20 | 10 | 90 | 60 | 60 | 30 | 60 | 50 | 40 | 30 | 10 | 10 | 50 | 0 | 0 | 70 | 0 | 0 |
| Lambsquarters | 20 | 0 | 0 | 0 | 90 | 20 | 0 | 0 | — | 90 | 40 | 100 | 0 | 90 | 60 | 90 | 100 | 80 | 90 | 90 | 70 | 0 | 0 | 0 | 100 | 0 | 0 |
| Morningglory | 0 | 0 | 0 | 0 | 0 | 10 | 0 | 0 | 0 | 10 | 0 | 0 | 0 | 20 | 0 | 30 | — | 10 | 50 | 10 | 20 | 0 | 0 | 0 | 0 | 0 | 0 |
| Pigweed | 90 | 20 | 0 | 0 | 90 | 60 | 0 | 0 | 40 | 100 | 90 | 60 | 0 | 100 | 70 | 100 | 90 | 80 | 90 | 90 | 60 | 0 | 0 | 0 | 100 | 0 | 0 |
| Rice | 0 | 0 | 0 | 0 | 0 | 30 | 0 | 0 | 30 | 20 | 0 | — | — | — | — | — | — | — | — | — | — | — | — | — | — | — | 0 |
| Surinam Grass | 20 | 0 | 0 | 0 | 0 | 20 | 0 | 0 | 40 | 10 | 10 | 80 | 30 | 90 | 20 | 80 | 100 | 50 | 50 | 30 | 20 | 10 | 0 | 0 | 50 | 0 | 0 |
| Velvetleaf | 20 | 10 | 0 | 0 | 20 | 0 | 0 | 0 | 20 | 30 | 10 | 100 | 40 | 30 | — | 70 | 60 | 70 | 100 | 40 | 30 | 0 | 0 | 0 | 30 | 0 | 0 |
| Wheat | | | | | | | — | — | — | — | — | 90 | 0 | 40 | 0 | 90 | 100 | 80 | 100 | 10 | 0 | — | 0 | 0 | 80 | 0 | — |
| | 125 g ai/ha | | | | | | | | | | | | | | | | | | | | | | | | | | |
| | Compounds | | | | | | | | | | | | | | | | | | | | | | | | | | |
| | 352 | 353 | 354 | 355 | 356 | 357 | 358 | 359 | 360 | 361 | 362 | 363 | 364 | 365 | 366 | 368 | 369 | 370 | 371 | 372 | 373 | 374 | 375 | 376 | 377 | 378 | 379 |
| Barnyardgrass | 70 | 40 | 0 | 70 | 60 | 30 | 70 | 0 | 10 | 80 | 10 | 0 | 100 | 90 | 70 | 40 | 30 | 10 | 0 | 80 | 0 | 0 | 20 | 80 | 0 | 30 | 0 |
| Cocklebur | 0 | 0 | 0 | 0 | 0 | 0 | 40 | 0 | 0 | 0 | 0 | 0 | 70 | 10 | 0 | 0 | 0 | 0 | 0 | 10 | 0 | 0 | 0 | 0 | 0 | 0 | 0 |
| Corn | 60 | — | 20 | 20 | 10 | 0 | 60 | 0 | 10 | 30 | 0 | 0 | 80 | 90 | 40 | 0 | 20 | 0 | 0 | 60 | 0 | 0 | 0 | 40 | 0 | 30 | 0 |
| Crabgrass, Large | 90 | 20 | 20 | 70 | 40 | 70 | 100 | 90 | 40 | 80 | 20 | 20 | 100 | 100 | 40 | 10 | 20 | 10 | 10 | 80 | 0 | 0 | 20 | 80 | 20 | — | 20 |
| Foxtail, Giant | 70 | 0 | 10 | 10 | 10 | 20 | 60 | 60 | 50 | 50 | 10 | 20 | 100 | 100 | 50 | 20 | 20 | 0 | 10 | 60 | 0 | 10 | 50 | 70 | 0 | 30 | 0 |
| Lambsquarters | 90 | — | — | 80 | 40 | 90 | 100 | 0 | 0 | 80 | 0 | 0 | 100 | 100 | — | — | 100 | 80 | — | 100 | 0 | — | 50 | 100 | 0 | 90 | 0 |
| Morningglory | 0 | — | 0 | 10 | 0 | 10 | 50 | 0 | 0 | 10 | 0 | 0 | 70 | 50 | 30 | 0 | 0 | 0 | 0 | 10 | 0 | 0 | 10 | 10 | 0 | 0 | 10 |
| Pigweed | 50 | 0 | 0 | 50 | 70 | 100 | 70 | 0 | 20 | 80 | 0 | 0 | 100 | 100 | 70 | 30 | 80 | 40 | 0 | — | 0 | 30 | 70 | 100 | 60 | 90 | 20 |
| Rice | — | — | — | — | — | — | — | | | | | | | | | | | | | | | | | | | | |
| Surinam Grass | 90 | 0 | 0 | 40 | 10 | 30 | 50 | 20 | 20 | 50 | 10 | 0 | 100 | 70 | 30 | 10 | 0 | 0 | 10 | 50 | 0 | 10 | 40 | 40 | 0 | 0 | 0 |
| Velvetleaf | 30 | 10 | 0 | 0 | 0 | 0 | 50 | 0 | 20 | 20 | 0 | 0 | 100 | 50 | 0 | 0 | 0 | 0 | 0 | 10 | 0 | 0 | 30 | 10 | 0 | 50 | 10 |
| Wheat | 10 | 0 | 0 | 10 | 0 | 40 | 40 | 10 | 30 | 10 | 0 | 10 | 100 | 70 | 30 | 0 | 0 | 0 | 0 | 30 | 0 | 0 | 10 | 50 | 0 | 0 | 0 |
| | 125 g ai/ha | | | | | | | | | | | | | | | | | | | | | | | | | | |
| | Compounds | | | | | | | | | | | | | | | | | | | | | | | | | | |
| | 380 | 381 | 382 | 383 | 384 | 385 | 386 | 387 | 388 | 389 | 390 | 391 | 392 | 393 | 394 | 395 | 396 | 397 | 398 | 399 | 400 | 401 | 402 | 403 | 404 | 405 | 406 |
| Barnyardgrass | 70 | 10 | 80 | 10 | 90 | 80 | 90 | 0 | 50 | 0 | 0 | 100 | 90 | 90 | 30 | 90 | 90 | 0 | 0 | 90 | 0 | 0 | 0 | 0 | 70 | 10 | 40 |
| Cocklebur | 0 | 0 | 0 | 0 | 0 | 0 | 20 | 0 | 0 | 0 | 0 | 70 | 40 | 30 | 20 | 60 | 40 | 0 | 0 | 50 | 0 | 0 | 0 | 0 | 40 | 0 | 0 |
| Corn | 0 | 0 | 0 | 0 | 80 | 30 | 40 | 0 | 30 | 0 | 0 | 90 | 70 | 40 | 0 | 90 | 80 | 0 | 0 | 90 | 0 | 0 | 0 | 0 | 0 | 0 | 0 |
| Crabgrass, Large | 90 | 70 | 80 | 50 | 90 | 90 | 100 | 0 | 80 | 0 | 10 | 100 | 100 | 90 | 40 | 100 | 100 | 0 | 0 | 100 | 0 | 0 | 0 | 0 | 70 | 30 | 90 |
| Foxtail, Giant | 70 | 10 | 80 | 10 | 90 | 30 | 90 | 0 | 20 | 0 | 0 | 90 | 80 | 80 | 10 | 100 | 100 | 0 | 0 | 100 | 0 | 0 | 0 | 0 | 30 | 0 | 20 |
| Lambsquarters | 90 | 40 | 100 | 40 | 100 | 90 | 100 | 0 | 90 | 0 | 30 | 100 | 100 | 90 | 10 | 100 | 100 | — | 0 | 100 | 0 | 0 | 0 | 0 | 90 | 70 | 90 |
| Morningglory | 10 | 0 | 10 | 0 | 10 | 20 | 50 | 0 | 0 | 0 | 0 | 90 | 90 | 20 | 0 | 80 | 60 | 0 | 0 | 10 | 0 | 0 | 0 | 0 | 60 | 30 | 10 |
| Pigweed | 100 | 0 | 50 | 0 | 90 | 70 | 100 | 0 | 80 | 0 | 0 | 100 | 100 | 100 | 10 | 100 | 90 | 0 | 0 | 100 | 0 | 0 | 0 | 0 | 70 | 10 | 50 |
| Surinam Grass | 40 | 20 | 50 | 10 | 100 | 80 | 80 | 0 | 20 | 0 | 0 | 100 | 70 | 80 | 10 | 90 | 90 | 0 | 0 | 90 | 0 | 0 | 0 | 0 | 40 | 0 | 20 |
| Velvetleaf | 10 | 10 | 10 | 10 | 70 | 60 | 60 | 0 | 10 | 0 | 0 | 80 | 70 | 50 | 0 | 80 | 80 | 0 | 0 | 80 | 0 | 0 | 0 | 0 | 20 | 0 | 10 |
| Wheat | 20 | 0 | 10 | 0 | 90 | 20 | 40 | 0 | 10 | 0 | 0 | 100 | 30 | 90 | 0 | 100 | 100 | 0 | 0 | 100 | 0 | 0 | 0 | 0 | 10 | 0 | 10 |
| | 125 g ai/ha | | | | | | | | | | | | | | | | | | | | | | | | | | |
| | Compounds | | | | | | | | | | | | | | | | | | | | | | | | | | |
| | 407 | 408 | 409 | 410 | 411 | 412 | 413 | 414 | 415 | 416 | 417 | 418 | 419 | 420 | 421 | 422 | 423 | 424 | 425 | 426 | 427 | 428 | 429 | 430 | 431 | 432 | 433 |
| Barnyardgrass | 40 | 70 | 90 | 100 | 60 | 40 | 40 | 90 | 10 | 70 | 80 | 70 | 100 | 90 | 0 | 0 | 90 | 20 | 10 | 10 | 0 | 0 | 100 | 90 | 10 | 10 | 50 |
| Cocklebur | 10 | 0 | 10 | 0 | 0 | 0 | — | 20 | 0 | 0 | 0 | 0 | 30 | 0 | 0 | 0 | 20 | 0 | 0 | 0 | 0 | 0 | 60 | 10 | 0 | 0 | 10 |
| Corn | 0 | 10 | 50 | 0 | 0 | 0 | 0 | 70 | 0 | 20 | 40 | 10 | 70 | 70 | 0 | 0 | 30 | 0 | 0 | 0 | 0 | 0 | 40 | 50 | 0 | 0 | 10 |
| Crabgrass, Large | 80 | 90 | 90 | 90 | 80 | 50 | 20 | 100 | 70 | 100 | 100 | 100 | 100 | 100 | 0 | 0 | 100 | 90 | 80 | 70 | 0 | 0 | 100 | 100 | 60 | 30 | 90 |
| Foxtail, Giant | 30 | 40 | 70 | 30 | 10 | 30 | 10 | 90 | 20 | 70 | 70 | 80 | 90 | 80 | 0 | 0 | 90 | 20 | 20 | 0 | 0 | 0 | 90 | 90 | 50 | 40 | 60 |
| Lambsquarters | 80 | 100 | 100 | 100 | 50 | 0 | — | 100 | 50 | — | 90 | 100 | 100 | 100 | 0 | 0 | 100 | 70 | 80 | 40 | 10 | 0 | 100 | 100 | 60 | 0 | 70 |
| Morningglory | 30 | 20 | 60 | 30 | 0 | 0 | 0 | 10 | 0 | 0 | 0 | 40 | 50 | 50 | 0 | 0 | 30 | 10 | 0 | 0 | 0 | 0 | 90 | 20 | 0 | 0 | 40 |
| Pigweed | 50 | 100 | 90 | 60 | 30 | 50 | 20 | 100 | 60 | 70 | 90 | 100 | 100 | 100 | 0 | 0 | 100 | 90 | 70 | 50 | 30 | 30 | 100 | 100 | 40 | 0 | 100 |
| Surinam Grass | 30 | 30 | 90 | 60 | 10 | 0 | 10 | 90 | 20 | 60 | 80 | 90 | 90 | 70 | 0 | 0 | 90 | 30 | 40 | 10 | 0 | 0 | 70 | 80 | 10 | 10 | 20 |
| Velvetleaf | 10 | 50 | 30 | 30 | 0 | 0 | 0 | 70 | 20 | 10 | 50 | 50 | 70 | 50 | 0 | 0 | 90 | 20 | 20 | 10 | 0 | 0 | 80 | 40 | 20 | 0 | 40 |
| Wheat | 10 | 40 | 80 | 10 | 0 | 0 | 0 | 80 | 0 | 20 | 30 | 40 | 90 | 50 | 0 | 0 | 80 | 10 | 0 | 0 | 0 | 0 | 60 | 70 | 0 | 0 | 0 |

| | 125 g ai/ha | | | | | | | | | | | | 62 g ai/ha | | | | | | | | | 31 g ai/ha | | | 16 g ai/ha |
|---|---|---|---|---|---|---|---|---|---|---|---|---|---|---|---|---|---|---|---|---|---|---|---|---|---|
| | Compounds | | | | | | | | | | | | Compounds | | | | | | | | | Compounds | | | Compound |
| | 434 | 435 | 436 | 437 | 438 | 439 | 440 | 441 | 442 | 443 | 444 | 445 | 54 | 72 | 73 | 74 | 129 | 152 | 191 | 192 | 251 | 49 | 168 | 346 | 251 |
| Barnyardgrass | 90 | 10 | 0 | 0 | 0 | 0 | 0 | 10 | 50 | 90 | 0 | 90 | 0 | 0 | 0 | 0 | 10 | 50 | 0 | 20 | 0 | 0 | 0 | 0 | 0 |
| Cocklebur | 10 | 0 | 0 | 0 | 0 | 0 | 0 | 0 | 0 | 0 | 0 | 10 | 0 | 0 | 0 | 0 | 0 | 0 | 0 | 0 | 0 | 0 | 0 | 0 | 0 |
| Corn | 60 | 0 | 0 | 0 | 0 | 0 | 0 | 0 | 10 | 30 | 0 | 40 | 0 | 0 | 0 | 0 | 0 | 0 | 0 | 0 | 0 | 0 | 0 | 0 | 0 |
| Crabgrass, Large | 100 | 40 | 80 | 80 | 0 | 0 | 0 | 80 | 80 | 100 | 0 | 100 | 0 | 0 | 0 | 0 | 40 | 90 | 10 | 30 | 0 | — | 90 | 0 | 0 |
| Foxtail, Giant | 90 | 10 | 0 | 0 | 0 | 0 | 0 | 30 | 40 | 80 | 0 | 90 | 0 | 0 | 10 | 0 | 0 | 90 | 0 | 10 | 0 | 0 | 0 | 0 | 0 |
| Lambsquarters | 100 | — | 30 | — | — | — | — | — | 100 | 100 | 0 | 100 | 0 | 0 | 0 | 0 | 0 | 100 | 50 | 60 | — | 0 | 50 | 0 | 0 |
| Morningglory | 80 | 0 | 0 | 0 | 0 | — | 0 | 0 | 0 | 10 | 0 | 70 | 0 | 0 | 0 | 0 | — | — | 0 | 0 | 0 | 0 | 0 | 0 | 0 |
| Pigweed | 100 | — | 50 | 30 | 0 | 0 | 0 | 60 | 70 | 100 | 0 | 100 | 0 | 0 | 0 | 0 | 0 | 100 | 10 | 30 | — | 60 | 90 | 0 | 0 |
| Rice | | | | | | | | | | | | | | | | | | | | | | 10 | 0 | — | 0 |
| Surinam Grass | 50 | 10 | 40 | 0 | 0 | 0 | 0 | — | 40 | 50 | 0 | 80 | 0 | 0 | 20 | 0 | — | 10 | 0 | 0 | 0 | 0 | 0 | 0 | 0 |
| Velvetleaf | 60 | 0 | 50 | 0 | 0 | 0 | 0 | 0 | 30 | 30 | 0 | 60 | 0 | 0 | 0 | 0 | 10 | 40 | 0 | 10 | 0 | 0 | 10 | 0 | 0 |
| Wheat | 70 | 0 | 0 | 0 | 0 | 0 | 0 | 0 | 10 | 80 | 0 | 80 | 0 | 0 | 0 | 0 | 0 | 80 | 0 | 0 | 0 | — | — | 0 | |

Test C

Seeds of plant species selected from bermudagrass (Cynodon dactylon (L.) Pers.), Surinam grass (Urochloa decumbens (Staph) R. D. Webster, previously named Brachiaria decumbens Stapf), cocklebur (Xanthium strumarium L.), corn (Zea mays L.), large crabgrass (Digitaria sanguinalis (L.) Scop.), woolly cupgrass (Eriochloa villosa (Thunb.) Kunth), giant foxtail (Setaria faberi Herrm.), goosegrass (Eleusine indica (L.) Gaertn.), johnsongrass (Sorghum halepense (L.) Pers.), kochia (Kochia scoparia (L.) Schrad.), lambsquarters (Chenopodium album L.), morningglory (Ipomoea coccinea L.), eastern black nightshade (Solanum ptycanthum Dunal), yellow nutsedge (Cyperus esculentus L.), pigweed (Amaranthus retroflexus L.), common ragweed (Ambrosia elatior L.), soybean (Glycine max (L.) Merr.), common (oilseed) sunflower (Helianthus annuus L.) and velvetleaf (Abutilon theophrasti Medik.) were planted and treated preemergence with test chemicals formulated in a non-phytotoxic solvent mixture which included a surfactant.

At the same time, plants selected from these crop and weed species and also winter barley (Hordeum vulgare L.), blackgrass (Alopecurus myosuroides Huds.), canarygrass (Phalaris minor Retz.), chickweed (Stellaria media (L.) Vill.), downy bromegrass (Bromus tectorum L.), green foxtail (Setaria viridis (L.) Beauv.), Italian ryegrass (Lolium multiflorum Lam.), wheat (Triticum aestivum L.), wild oat (Avenafatua L.) and windgrass (Apera spica-venti (L.) Beauv.) were treated with postemergence applications of some of the test chemicals formulated in the same manner. Plants ranged in height from 2 to 18 cm (1- to 4-leaf stage) for postemergence treatments. Plant species in the flooded paddy test consisted of rice (Oryza sativa), smallflower umbrella sedge (Cyperus difformis L.), ducksalad (Heteranthera limosa (Sw.) Willd.) and barnyardgrass (Echinochloa crus-galli (L.) Beauv.) grown to the 2-leaf stage for testing. Treated plants and controls were maintained in a greenhouse for 12 to 14 days, after which time all species were compared to controls and visually evaluated. Plant response ratings, summarized in Table C, are based on a scale of 0 to 100 where 0 is no effect and 100 is complete control. A dash (-) response means no test result.

TABLE C
Flooded Paddy
500 g ai/ha
Compounds

| | 1 | 2 | 3 | 4 | 5 | 6 | 7 | 9 | 10 | 11 | 13 | 14 | 15 | 16 | 17 | 18 | 20 | 21 | 27 | 28 | 29 | 31 | 34 | 43 | 45 | 47 | 49 |
|---|---|---|---|---|---|---|---|---|---|---|---|---|---|---|---|---|---|---|---|---|---|---|---|---|---|---|---|
| Barnyardgrass | 75 | 90 | 60 | 80 | 10 | 90 | 90 | 70 | 80 | 90 | 90 | 95 | 75 | 95 | 80 | 85 | 15 | 70 | 85 | 60 | 85 | 75 | 65 | 50 | 80 | 40 | 80 |
| Ducksalad | 95 | 95 | 90 | 90 | 70 | 100 | 80 | 95 | 90 | 80 | 85 | 85 | 30 | 90 | 85 | 70 | 25 | 65 | 80 | 70 | 85 | 60 | 75 | 70 | 60 | 80 | 80 |
| Rice | 95 | 85 | 70 | 75 | 0 | 90 | 75 | 80 | 90 | 80 | 70 | 85 | 50 | 90 | 80 | 80 | 30 | 45 | 80 | 35 | 80 | 50 | 55 | 20 | 65 | 50 | 90 |
| Sedge, Umbrella | — | 95 | 90 | 95 | 70 | 90 | 80 | — | 85 | 80 | 90 | 95 | 70 | 90 | 85 | 85 | 80 | 50 | 80 | 25 | 90 | 55 | 90 | 80 | 80 | 80 | 80 |
| | 500 g ai/ha | | | | | | | | | | | | | | | | | | | | | | | | | | |
| | Compounds | | | | | | | | | | | | | | | | | | | | | | | | | | |
| | 51 | 55 | 56 | 58 | 62 | 63 | 64 | 65 | 67 | 75 | 76 | 79 | 80 | 81 | 88 | 95 | 96 | 101 | 102 | 106 | 108 | 112 | 115 | 117 | 118 | 122 | 126 |
| Barnyardgrass | 75 | 75 | 80 | 85 | 55 | 50 | 35 | 60 | 25 | 70 | 90 | 80 | 60 | 30 | 45 | 60 | 80 | 50 | 70 | 0 | 70 | 30 | 75 | 50 | 50 | 80 | 25 |
| Ducksalad | 85 | 80 | 75 | 60 | 90 | 85 | 40 | 85 | 40 | 70 | 80 | 55 | 40 | 20 | 75 | 90 | 90 | 75 | 90 | 0 | 90 | 100 | 90 | 60 | 50 | 80 | 30 |
| Rice | 50 | 60 | 35 | 75 | 65 | 50 | 15 | 30 | 30 | 65 | 90 | 30 | 40 | 40 | 45 | 65 | 75 | 25 | 60 | 20 | 50 | 30 | 75 | 65 | 50 | 50 | 0 |
| Sedge, Umbrella | 80 | 80 | 90 | 65 | 85 | 90 | 75 | 80 | 75 | 85 | 90 | 85 | 70 | 80 | 80 | 95 | 95 | 90 | 80 | 80 | 80 | 50 | 95 | 80 | 50 | 80 | 50 |
| | 500 g ai/ha | | | | | | | | | | | | | | | | | | | | | | | | | | |
| | Compounds | | | | | | | | | | | | | | | | | | | | | | | | | | |
| | 127 | 128 | 131 | 134 | 136 | 138 | 139 | 140 | 141 | 142 | 144 | 146 | 152 | 155 | 158 | 159 | 160 | 161 | 162 | 181 | 183 | 186 | 187 | 188 | 192 | 193 | 194 |
| Barnyardgrass | 55 | 65 | 30 | 50 | 80 | 30 | 65 | 15 | 10 | 90 | 60 | 20 | 85 | 65 | 35 | 80 | 85 | 75 | 90 | 0 | 0 | 70 | 75 | 85 | 70 | 90 | 25 |
| Ducksalad | 80 | 95 | 80 | 90 | 90 | 70 | 90 | 20 | 0 | 100 | 80 | 20 | 85 | 90 | 50 | 100 | 90 | 90 | 100 | 80 | 20 | 80 | 90 | 85 | 60 | 90 | 40 |
| Rice | 25 | 70 | 10 | 60 | 70 | 40 | 60 | 0 | 0 | 70 | 30 | 20 | 80 | 35 | 60 | 90 | 95 | 65 | 75 | 0 | 0 | 65 | 65 | 75 | 70 | 75 | 45 |
| Sedge, Umbrella | 85 | 95 | 80 | 85 | 85 | 95 | 85 | 65 | 45 | 90 | 90 | 90 | 85 | 75 | 80 | 80 | 85 | 40 | — | 85 | 50 | 85 | 85 | 85 | 90 | 85 | 75 |
| | 500 g ai/ha | | | | | | | | | | | | | | | | | | | | | | | | | | |
| | Compounds | | | | | | | | | | | | | | | | | | | | | | | | | | |
| | 207 | 209 | 210 | 215 | 216 | 219 | 222 | 223 | 225 | 226 | 227 | 228 | 229 | 231 | 232 | 235 | 236 | 241 | 242 | 243 | 244 | 245 | 247 | 252 | 255 | 256 | 258 |
| Barnyardgrass | 85 | 80 | 85 | 50 | 30 | 0 | 95 | 45 | 90 | 60 | 0 | 95 | 80 | 80 | 15 | 0 | 40 | 30 | 95 | 85 | 80 | 70 | 0 | 45 | 35 | 0 | 45 |
| Ducksalad | 85 | 80 | 90 | 80 | 40 | 0 | 95 | 55 | 85 | 65 | 55 | 90 | 85 | 80 | 80 | 75 | 80 | 15 | 95 | 90 | 95 | 75 | 80 | 80 | 40 | 65 | 90 |
| Rice | 60 | 65 | 75 | 20 | 30 | 0 | 85 | 75 | 80 | 70 | 55 | 85 | 90 | 80 | 60 | 35 | 70 | 65 | 90 | 90 | 75 | 70 | 75 | 60 | 50 | 15 | 70 |
| Sedge, Umbrella | 90 | 80 | 90 | 90 | 50 | 20 | 90 | 85 | 80 | 70 | 15 | 85 | 80 | 75 | 85 | 80 | 55 | 85 | 85 | 80 | 85 | 70 | 75 | 75 | 45 | 60 | 80 |
| | 500 g ai/ha | | | | | | | | | | | | | | | | | | | | | | | | | | |
| | Compounds | | | | | | | | | | | | | | | | | | | | | | | | | | |
| | 259 | 260 | 263 | 266 | 268 | 269 | 272 | 273 | 274 | 276 | 278 | 281 | 283 | 285 | 286 | 287 | 290 | 293 | 294 | 295 | 296 | 298 | 299 | 300 | 301 | 302 | 303 |
| Barnyardgrass | 0 | 35 | 0 | 70 | 95 | 90 | 30 | 70 | 60 | 70 | 25 | 65 | 10 | 75 | 80 | 25 | 40 | 80 | 100 | 40 | 65 | 90 | 95 | 95 | 95 | 35 | 95 |
| Ducksalad | 75 | 85 | 90 | 95 | 90 | 85 | 85 | 75 | 20 | 85 | 20 | 85 | 0 | 90 | 90 | 45 | 70 | 95 | 95 | 30 | 70 | 90 | 95 | 90 | 95 | 85 | 85 |
| Rice | 0 | 15 | 25 | 80 | 90 | 80 | 20 | 60 | 45 | 70 | 30 | 50 | 0 | 65 | 75 | 55 | 45 | 80 | 90 | 40 | 60 | 75 | 85 | 75 | 85 | 55 | 85 |
| Sedge, Umbrella | 70 | 65 | 50 | 80 | 95 | 85 | 75 | 75 | 75 | 85 | 75 | 90 | 0 | 65 | 90 | 75 | 70 | 90 | 90 | 80 | 85 | 80 | 95 | 90 | 95 | 85 | 90 |
| | 500 g ai/ha | | | | | | | | | | | | | | | | | | | | | | | | | | |
| | Compounds | | | | | | | | | | | | | | | | | | | | | | | | | | |
| | 304 | 305 | 306 | 307 | 308 | 309 | 310 | 319 | 331 | 332 | 333 | 334 | 335 | 336 | 340 | 341 | 342 | 343 | 344 | 349 | 352 | 353 | 357 | 358 | 359 | 368 | 369 |
| Barnyardgrass | 95 | 100 | 70 | 95 | 65 | 90 | 90 | 65 | 65 | 60 | 20 | 95 | 65 | 80 | 70 | 60 | 60 | 80 | 0 | 95 | 90 | 20 | 55 | 85 | 75 | 30 | 65 |
| Ducksalad | 95 | 0 | 90 | 90 | 75 | 90 | 90 | 85 | 85 | 95 | 25 | 90 | 70 | 90 | 90 | 60 | 80 | 90 | 45 | 95 | 95 | 30 | 95 | 95 | 90 | — | 80 |
| Rice | 75 | 85 | 70 | 80 | 70 | 75 | 80 | 70 | 65 | 70 | 20 | 85 | 55 | 75 | 80 | 70 | 80 | 80 | 15 | 70 | 70 | 40 | 70 | 85 | 80 | 70 | 65 |
| Sedge, Umbrella | 95 | 90 | 90 | 85 | 80 | 80 | 85 | 80 | 65 | 85 | 80 | 95 | 95 | 90 | 90 | 60 | 80 | 90 | 85 | 95 | 95 | 50 | 95 | 85 | 95 | — | — |
| | 500 g ai/ha | | | | | | | | | | | | | | | | | | | | | | | | | | |
| | Compounds | | | | | | | | | | | | | | | | | | | | | | | | | | |
| | 372 | 376 | 378 | 380 | 382 | 383 | 384 | 385 | 386 | 388 | 391 | 392 | 393 | 394 | 395 | 396 | 399 | 404 | 405 | 406 | 407 | 408 | 409 | 410 | 411 | 412 | 413 |
| Barnyardgrass | 80 | 75 | 50 | 70 | 75 | 40 | 95 | 85 | 95 | 65 | 95 | 90 | 95 | 20 | 90 | 80 | 90 | 30 | 20 | 40 | 20 | 70 | 60 | 65 | 45 | 45 | 80 |
| Ducksalad | 85 | 85 | 75 | 85 | 85 | 95 | 85 | 75 | 85 | 80 | 95 | 85 | 90 | 45 | 55 | 75 | 85 | 30 | 30 | 75 | 40 | 95 | 85 | 60 | 80 | 90 | 70 |
| Rice | 90 | 80 | 60 | 75 | 75 | 50 | 80 | 80 | 75 | 70 | 80 | 75 | 85 | 60 | 75 | 80 | 75 | 50 | 20 | 45 | 40 | 55 | 60 | 60 | 35 | 50 | 70 |
| Sedge, Umbrella | 85 | 85 | 80 | 90 | 90 | 90 | 80 | 85 | 80 | 80 | 95 | 85 | 85 | 70 | 85 | 90 | 85 | 85 | 70 | 75 | 75 | 85 | 90 | 85 | 85 | 95 | 75 |
| | 250 g ai/ha | | | | | | | | | | | | | | | | | | | | | | | | | | |
| | Compounds | | | | | | | | | | | | | | | | | | | | | | | | | | |
| | 1 | 2 | 3 | 4 | 5 | 6 | 7 | 9 | 10 | 11 | 13 | 14 | 15 | 16 | 17 | 18 | 20 | 21 | 27 | 28 | 29 | 31 | 34 | 43 | 45 | 47 | 49 |
| Barnyardgrass | 65 | 75 | 20 | 65 | 0 | 90 | 75 | 65 | 70 | 70 | 80 | 90 | 25 | 90 | 75 | 65 | 0 | 15 | 60 | 35 | 60 | 70 | 45 | 40 | 60 | 40 | 70 |
| Ducksalad | 95 | 85 | 80 | 90 | 50 | 90 | 80 | 95 | 90 | 80 | 75 | 80 | 20 | 85 | 80 | 65 | 20 | 40 | 80 | 40 | 75 | 40 | 25 | 60 | 60 | 80 | 80 |
| Rice | 75 | 65 | 10 | 65 | 0 | 80 | 65 | 60 | 70 | 80 | 65 | 75 | 25 | 80 | 80 | 50 | 0 | 20 | 65 | 0 | 60 | 40 | 50 | 20 | 35 | 20 | 70 |
| Sedge, Umbrella | — | 85 | 80 | 85 | 60 | 90 | 80 | — | 75 | 70 | 80 | 90 | 55 | 90 | 80 | 80 | 50 | 30 | 80 | 20 | 60 | 40 | 80 | 70 | 80 | 80 | 80 |
| | 250 g ai/ha | | | | | | | | | | | | | | | | | | | | | | | | | | |
| | Compounds | | | | | | | | | | | | | | | | | | | | | | | | | | |
| | 51 | 55 | 56 | 58 | 62 | 63 | 64 | 65 | 67 | 75 | 76 | 79 | 80 | 81 | 88 | 95 | 96 | 101 | 102 | 106 | 108 | 112 | 115 | 117 | 118 | 122 | 126 |
| Barnyardgrass | 20 | 25 | 65 | 35 | 30 | 20 | 0 | 30 | 10 | 65 | 80 | 25 | 40 | 0 | 15 | 55 | 70 | 30 | 50 | 0 | 60 | 10 | 65 | 50 | 20 | 60 | 20 |
| Ducksalad | 75 | 55 | 70 | 20 | 90 | 70 | 25 | 75 | 40 | 65 | 75 | 15 | 20 | 20 | 40 | 75 | 75 | 40 | 90 | 0 | 80 | 0 | 85 | 0 | 50 | 80 | 25 |
| Rice | 15 | 15 | 25 | 35 | 45 | 15 | 0 | 25 | 0 | 55 | 85 | 20 | 40 | 30 | 20 | 55 | 70 | 0 | 40 | 0 | 20 | 10 | 75 | 45 | 40 | 10 | 0 |
| Sedge, Umbrella | 65 | 75 | 90 | 50 | 80 | 80 | 35 | 75 | 75 | 80 | 85 | 80 | 70 | 80 | 80 | 95 | 90 | 80 | 70 | 30 | 80 | 25 | 90 | 75 | 50 | 70 | 20 |
| | 250 g ai/ha | | | | | | | | | | | | | | | | | | | | | | | | | | |
| | Compounds | | | | | | | | | | | | | | | | | | | | | | | | | | |
| | 127 | 128 | 131 | 134 | 136 | 138 | 139 | 140 | 141 | 142 | 144 | 146 | 152 | 155 | 158 | 159 | 160 | 161 | 162 | 181 | 183 | 186 | 187 | 188 | 192 | 193 | 194 |
| Barnyardgrass | 30 | 60 | 10 | 50 | 40 | 15 | 55 | 0 | 0 | 80 | 50 | 0 | 75 | 50 | 15 | 70 | 70 | 30 | 75 | 0 | 0 | 45 | 60 | 75 | 0 | 85 | 0 |
| Ducksalad | 20 | 95 | 45 | 90 | 45 | 55 | 85 | 0 | 0 | 95 | 70 | 20 | 85 | 85 | 20 | 90 | 90 | 80 | 95 | 0 | 10 | 65 | 85 | 75 | 25 | 90 | 0 |
| Rice | 25 | 70 | 0 | 55 | 50 | 5 | 45 | 0 | 0 | 70 | 30 | 0 | 75 | 0 | 35 | 80 | 75 | 60 | 70 | 0 | 0 | 60 | 50 | 65 | 45 | 70 | 25 |
| Sedge, Umbrella | 80 | 95 | 70 | 85 | 85 | 75 | 85 | 45 | 30 | 80 | 80 | 90 | 85 | 65 | 70 | 60 | 80 | 25 | 75 | 80 | 10 | 80 | 80 | 80 | 85 | 80 | 35 |
| | 250 g ai/ha | | | | | | | | | | | | | | | | | | | | | | | | | | |
| | Compounds | | | | | | | | | | | | | | | | | | | | | | | | | | |
| | 207 | 209 | 210 | 215 | 216 | 219 | 222 | 223 | 225 | 226 | 227 | 228 | 229 | 231 | 232 | 235 | 236 | 241 | 242 | 243 | 244 | 245 | 247 | 252 | 255 | 256 | 258 |
| Barnyardgrass | 75 | 70 | 85 | 40 | 0 | 0 | 90 | 0 | 80 | 50 | 0 | 95 | 75 | 75 | 0 | 0 | 15 | 0 | 90 | 75 | 75 | 45 | 0 | 15 | 25 | 0 | 15 |
| Ducksalad | 80 | 65 | 85 | 60 | 30 | 0 | 85 | 25 | 85 | 40 | 0 | 90 | 80 | 70 | 30 | 0 | 55 | 0 | 95 | 90 | 85 | 75 | 0 | 15 | 35 | 15 | 70 |
| Rice | 50 | 45 | 75 | 0 | 0 | 0 | 85 | 45 | 75 | 45 | 25 | 80 | 80 | 75 | 35 | 25 | 60 | 30 | 90 | 80 | 75 | 70 | 20 | 55 | 45 | 0 | 5 |
| Sedge, Umbrella | 75 | 65 | 85 | 70 | 30 | 0 | 85 | 65 | 75 | 40 | 0 | 80 | 80 | 65 | 55 | 55 | 45 | 55 | 85 | 80 | 70 | 70 | 15 | 50 | 35 | 60 | 70 |
| | 250 g ai/ha | | | | | | | | | | | | | | | | | | | | | | | | | | |
| | Compounds | | | | | | | | | | | | | | | | | | | | | | | | | | |
| | 259 | 260 | 263 | 266 | 268 | 269 | 272 | 273 | 274 | 276 | 278 | 281 | 283 | 285 | 286 | 287 | 290 | 293 | 294 | 295 | 296 | 298 | 299 | 300 | 301 | 302 | 303 |
| Barnyardgrass | 0 | 20 | 0 | 50 | 95 | 75 | 10 | 60 | 30 | 50 | 0 | 30 | 0 | 55 | 70 | 0 | 25 | 70 | 95 | 25 | 50 | 65 | 95 | 90 | 90 | 10 | 80 |
| Ducksalad | 65 | 75 | 75 | 75 | 85 | 80 | 40 | 75 | 0 | 70 | 0 | 70 | 0 | 90 | 90 | 40 | 45 | 80 | 95 | 25 | 40 | 80 | 95 | 85 | 90 | 30 | 80 |
| Rice | 0 | 0 | 0 | 65 | 90 | 75 | 15 | 50 | 15 | 50 | 15 | 15 | 0 | 45 | 65 | 25 | 15 | 65 | 85 | 40 | 40 | 65 | 80 | 70 | 75 | 0 | 80 |
| Sedge, Umbrella | 55 | 65 | 30 | 55 | 80 | 80 | 75 | 75 | 70 | 80 | 65 | 40 | 0 | 50 | 90 | 70 | 65 | 80 | 90 | 80 | 75 | 70 | 95 | 85 | 90 | 75 | 85 |
| | 250 g ai/ha | | | | | | | | | | | | | | | | | | | | | | | | | | |
| | Compounds | | | | | | | | | | | | | | | | | | | | | | | | | | |
| | 304 | 305 | 306 | 307 | 308 | 309 | 310 | 319 | 331 | 332 | 333 | 334 | 335 | 336 | 340 | 341 | 342 | 343 | 344 | 349 | 352 | 353 | 357 | 358 | 359 | 368 | 369 |
| Barnyardgrass | 85 | 95 | 55 | 80 | 25 | 85 | 70 | 50 | 40 | 50 | 10 | 95 | 40 | 60 | 60 | 50 | 50 | 60 | 0 | 90 | 65 | 20 | 30 | 75 | 75 | 0 | 50 |
| Ducksalad | 95 | 0 | 80 | 90 | 15 | 90 | 80 | 45 | 65 | 95 | 0 | 85 | 50 | 75 | 70 | 60 | 80 | 90 | 20 | 85 | 85 | 30 | 45 | 90 | 85 | 90 | 80 |
| Rice | 70 | 85 | 55 | 65 | 65 | 75 | 70 | 35 | 50 | 45 | 15 | 80 | 55 | 75 | 70 | 60 | 60 | 70 | 0 | 60 | 60 | 20 | 45 | 70 | 65 | 60 | 60 |
| Sedge, Umbrella | 95 | 90 | 90 | 80 | 70 | 80 | 85 | 70 | 25 | 85 | 30 | 90 | 90 | 85 | 70 | 60 | 70 | 80 | 75 | 90 | 90 | 50 | 90 | 85 | 90 | 75 | 85 |
| | 250 g ai/ha | | | | | | | | | | | | | | | | | | | | | | | | | | |
| | Compounds | | | | | | | | | | | | | | | | | | | | | | | | | | |
| | 372 | 376 | 378 | 380 | 382 | 383 | 384 | 385 | 386 | 388 | 391 | 392 | 393 | 394 | 395 | 396 | 399 | 404 | 405 | 406 | 407 | 408 | 409 | 410 | 411 | 412 | 413 |
| Barnyardgrass | 75 | 70 | 0 | 65 | 65 | 15 | 90 | 80 | 80 | 35 | 90 | 80 | 75 | 0 | 80 | 75 | 85 | 20 | 0 | 30 | 0 | 60 | 40 | 60 | 40 | 35 | 70 |
| Ducksalad | 85 | 85 | 20 | 75 | 80 | 45 | 80 | 70 | 85 | 45 | 85 | 85 | 80 | 10 | 55 | 65 | 85 | 20 | 20 | 50 | 40 | 95 | 80 | 60 | 70 | 60 | 70 |
| Rice | 90 | 75 | 30 | 70 | 70 | 35 | 80 | 65 | 70 | 65 | 75 | 75 | 80 | 45 | 70 | 75 | 75 | 45 | 0 | 40 | 20 | 45 | 50 | 60 | 35 | 20 | 55 |
| Sedge, Umbrella | 85 | 85 | 55 | 85 | 85 | 65 | 80 | 80 | 80 | 75 | 85 | 85 | 80 | 60 | 85 | 85 | 85 | 80 | 70 | 75 | 75 | 85 | 85 | 80 | 80 | 80 | 75 |
| | 125 g ai/ha | | | | | | | | | | | | | | | | | | | | | | | | | | |
| | Compounds | | | | | | | | | | | | | | | | | | | | | | | | | | |
| | 1 | 2 | 3 | 4 | 5 | 6 | 7 | 9 | 10 | 11 | 13 | 14 | 15 | 16 | 17 | 18 | 20 | 21 | 27 | 28 | 29 | 31 | 34 | 43 | 45 | 47 | 48 |
| Barnyardgrass | 60 | 75 | 0 | 50 | 0 | 90 | 50 | 50 | 65 | 20 | 55 | 80 | 0 | 80 | 65 | 20 | 0 | 0 | 35 | 0 | 25 | 30 | 35 | 0 | 30 | 0 | 0 |
| Ducksalad | 90 | 80 | 65 | 75 | 15 | 90 | 60 | 95 | 90 | 80 | 35 | 75 | 0 | 80 | 80 | 40 | 10 | 40 | 60 | 0 | 45 | 20 | 25 | 0 | 0 | 70 | 40 |
| Rice | 70 | 50 | 5 | 50 | 0 | 80 | 55 | 55 | 60 | 40 | 50 | 70 | 0 | 75 | 70 | 40 | 0 | 0 | 20 | 0 | 25 | 10 | 0 | 0 | 30 | 20 | 20 |
| Sedge, Umbrella | 80 | 75 | 65 | 75 | 15 | 90 | 80 | — | 70 | 60 | 75 | 80 | 10 | 85 | 45 | 60 | 15 | 25 | 70 | 15 | 60 | 35 | 80 | 50 | 60 | 70 | 60 |
| | 125 g ai/ha | | | | | | | | | | | | | | | | | | | | | | | | | | |
| | Compounds | | | | | | | | | | | | | | | | | | | | | | | | | | |
| | 49 | 51 | 55 | 56 | 58 | 62 | 63 | 64 | 65 | 67 | 75 | 76 | 79 | 80 | 81 | 88 | 95 | 96 | 101 | 102 | 106 | 108 | 112 | 115 | 117 | 118 | 122 |
| Barnyardgrass | 60 | 0 | 0 | 20 | 10 | 10 | 0 | 0 | 0 | 0 | 25 | 70 | 25 | 20 | 0 | 0 | 45 | 45 | 20 | 50 | 0 | 40 | 0 | 45 | 20 | 20 | 50 |
| Ducksalad | 50 | 55 | 0 | 55 | 15 | 35 | 50 | 10 | 45 | 30 | 30 | 50 | 0 | 20 | 0 | 15 | 25 | 45 | 0 | 80 | 0 | 70 | 0 | 75 | 0 | 50 | 80 |
| Rice | 25 | 0 | 0 | 15 | 35 | 25 | 0 | 0 | 0 | 0 | 25 | 80 | 20 | 40 | 20 | 0 | 10 | 45 | 0 | 30 | 0 | 0 | 0 | 65 | 15 | 30 | 10 |
| Sedge, Umbrella | 45 | 50 | 20 | 85 | 20 | 60 | 70 | 25 | 20 | 50 | 75 | 85 | 75 | 70 | 70 | 50 | 90 | 90 | 75 | 70 | 0 | 80 | 0 | 85 | 55 | 50 | 60 |
| | 125 g ai/ha | | | | | | | | | | | | | | | | | | | | | | | | | | |
| | Compounds | | | | | | | | | | | | | | | | | | | | | | | | | | |
| | 126 | 127 | 128 | 131 | 134 | 136 | 138 | 139 | 140 | 141 | 142 | 144 | 146 | 147 | 152 | 155 | 158 | 159 | 160 | 161 | 162 | 181 | 183 | 186 | 187 | 188 | 192 |
| Barnyardgrass | 0 | 30 | 35 | 0 | 25 | 25 | 0 | 40 | 0 | 0 | 65 | 20 | 0 | 0 | 75 | 25 | 0 | 50 | 60 | 0 | 65 | 0 | 0 | 30 | 25 | 55 | 0 |
| Ducksalad | 10 | 15 | 95 | 20 | 70 | 0 | 20 | 70 | 0 | 0 | 90 | 50 | 0 | 0 | 85 | 80 | 0 | 90 | 85 | 65 | 85 | 0 | 0 | 40 | 40 | 65 | 0 |
| Rice | 0 | 25 | 40 | 0 | 45 | 35 | 5 | 35 | 0 | 0 | 70 | 20 | 0 | 0 | 75 | 0 | 0 | 60 | 70 | 10 | 60 | 0 | 0 | 55 | 30 | 45 | 15 |
| Sedge, Umbrella | 20 | 80 | 90 | 65 | 75 | 80 | 40 | 80 | 20 | 0 | 80 | 80 | 80 | 50 | 80 | 30 | 45 | 40 | 70 | 0 | — | 20 | 0 | 80 | 65 | 75 | 75 |
| | 125 g ai/ha | | | | | | | | | | | | | | | | | | | | | | | | | | |
| | Compounds | | | | | | | | | | | | | | | | | | | | | | | | | | |
| | 193 | 194 | 207 | 209 | 210 | 215 | 216 | 219 | 222 | 223 | 225 | 226 | 227 | 228 | 229 | 231 | 232 | 235 | 236 | 241 | 242 | 243 | 244 | 245 | 247 | 252 | 255 |
| Barnyardgrass | 55 | 0 | 65 | 55 | 70 | 0 | 0 | 0 | 75 | 0 | 60 | 0 | 0 | 85 | 45 | 45 | 0 | 0 | 0 | 0 | 75 | 65 | 70 | 35 | 0 | 0 | 10 |
| Ducksalad | 70 | 0 | 80 | 65 | 55 | 40 | 20 | 0 | 75 | 0 | 75 | 0 | 0 | 85 | 0 | 60 | 0 | 0 | 40 | 0 | 95 | 85 | 85 | 75 | 0 | 0 | 10 |
| Rice | 50 | 25 | 45 | 45 | 55 | 0 | 0 | 0 | 70 | 0 | 70 | 25 | 0 | 80 | 35 | 55 | 20 | 0 | 30 | 0 | 85 | 75 | 70 | 65 | 0 | 45 | 0 |
| Sedge, Umbrella | 75 | 0 | 75 | 55 | 80 | 40 | 30 | 0 | 75 | 0 | 75 | 10 | 0 | 80 | 40 | 55 | 35 | 50 | 20 | 0 | 75 | 80 | 65 | 70 | 0 | 40 | 20 |
| | 125 g ai/ha | | | | | | | | | | | | | | | | | | | | | | | | | | |
| | Compounds | | | | | | | | | | | | | | | | | | | | | | | | | | |
| | 256 | 258 | 259 | 260 | 263 | 266 | 268 | 269 | 272 | 273 | 274 | 276 | 278 | 281 | 283 | 285 | 286 | 287 | 290 | 293 | 294 | 295 | 296 | 298 | 299 | 300 | 301 |
| Barnyardgrass | 0 | 0 | 0 | 0 | 0 | 25 | 90 | 65 | 0 | 40 | 10 | 15 | 0 | 20 | 0 | 25 | 55 | 0 | 0 | 20 | 90 | 15 | 25 | 50 | 85 | 50 | 75 |
| Ducksalad | 0 | 45 | 60 | 70 | 0 | 15 | 70 | 80 | 20 | 50 | 0 | 20 | 0 | 0 | 0 | 80 | 75 | 30 | 0 | 65 | 95 | 0 | 0 | 40 | 95 | 80 | 90 |
| Rice | 0 | 0 | 0 | 0 | 0 | 10 | 85 | 50 | 10 | 40 | 0 | 15 | 0 | 0 | 0 | 40 | 55 | 20 | 0 | 30 | 75 | 10 | 20 | 60 | 80 | 55 | 55 |
| Sedge, Umbrella | 0 | 20 | 35 | 40 | 0 | 20 | 70 | 80 | 65 | 75 | 50 | 75 | 25 | 20 | 0 | 35 | 70 | 35 | 25 | 75 | 85 | 80 | 75 | 0 | 95 | 80 | 90 |
| | 125 g ai/ha | | | | | | | | | | | | | | | | | | | | | | | | | | |
| | Compounds | | | | | | | | | | | | | | | | | | | | | | | | | | |
| | 302 | 303 | 304 | 305 | 306 | 307 | 308 | 309 | 310 | 313 | 319 | 331 | 332 | 333 | 334 | 335 | 336 | 340 | 341 | 342 | 343 | 344 | 349 | 352 | 353 | 357 | 358 |
| Barnyardgrass | 0 | 45 | 50 | 85 | 45 | 65 | 0 | 60 | 50 | 0 | 25 | 10 | 25 | 0 | 90 | 25 | 0 | 50 | 30 | 20 | 20 | 0 | 70 | 45 | 0 | 0 | 60 |
| Ducksalad | 25 | 75 | 85 | 0 | 80 | 70 | 0 | 90 | 75 | 30 | 0 | 50 | 90 | 0 | 85 | 35 | 55 | 70 | 50 | 50 | 80 | 0 | 85 | 65 | 0 | 30 | 80 |
| Rice | 0 | 65 | 60 | 80 | 40 | 60 | 25 | 70 | 60 | 20 | 35 | 15 | 0 | 0 | 75 | 25 | 45 | 40 | 50 | 30 | 50 | 0 | 55 | 45 | 0 | 0 | 65 |
| Sedge, Umbrella | 50 | 85 | 90 | 90 | 90 | 70 | 25 | 80 | 85 | 70 | 35 | 20 | 80 | 15 | 90 | 85 | 80 | 70 | 50 | 50 | 40 | 75 | 85 | 75 | 0 | 85 | 85 |
| | 125 g ai/ha | | | | | | | | | | | | | | | | | | | | | | | | | | |
| | Compounds | | | | | | | | | | | | | | | | | | | | | | | | | | |
| | 359 | 368 | 369 | 372 | 376 | 378 | 380 | 382 | 383 | 384 | 385 | 386 | 388 | 391 | 392 | 393 | 394 | 395 | 396 | 399 | 404 | 405 | 406 | 407 | 408 | 409 | 410 |
| Barnyardgrass | 55 | 0 | 45 | 70 | 65 | 0 | 45 | 25 | 0 | 75 | 55 | 50 | 10 | 85 | 45 | 65 | 0 | 80 | 75 | 65 | 0 | 0 | 25 | 0 | 45 | 20 | 20 |
| Ducksalad | 80 | 80 | 80 | 85 | 85 | 0 | 50 | 65 | 45 | 80 | — | 60 | 0 | 85 | 75 | 75 | 0 | 20 | 0 | 75 | 10 | 0 | 20 | 40 | 85 | 30 | 40 |
| Rice | 45 | 45 | 60 | 85 | 75 | 10 | 65 | 55 | 5 | 65 | 50 | 65 | 55 | 65 | 60 | 75 | 0 | 70 | 70 | 65 | 0 | 0 | 35 | 0 | 35 | 40 | 40 |
| Sedge, Umbrella | 90 | 75 | 75 | 85 | 85 | 15 | 85 | 80 | 45 | 80 | 75 | 75 | 70 | 80 | 75 | 75 | 35 | 80 | 75 | 70 | 75 | 50 | 75 | 60 | 80 | 75 | 75 |
| | 125 g ai/ha | | | 62 g ai/ha | | | | | | | | | | | | | | | | | | | | | | | |
| | Compounds | | | Compounds | | | | | | | | | | | | | | | | | | | | | | | |
| | 411 | 412 | 413 | 1 | 2 | 3 | 4 | 5 | 6 | 7 | 9 | 10 | 11 | 13 | 14 | 15 | 16 | 17 | 18 | 20 | 21 | 27 | 28 | 29 | 31 | 34 | 43 |
| Barnyardgrass | 25 | 20 | 25 | 50 | 15 | 0 | 20 | 0 | 60 | 10 | 35 | 25 | 0 | 25 | 65 | 0 | 80 | 50 | 0 | 0 | 0 | 30 | 0 | 10 | 0 | 0 | 0 |
| Ducksalad | 50 | 30 | 45 | 90 | 80 | 15 | 50 | 15 | 80 | 20 | 95 | 80 | 50 | 25 | 60 | 0 | 75 | 75 | 20 | 0 | 0 | 45 | 0 | 0 | 10 | 15 | 0 |
| Rice | 25 | 20 | 45 | 65 | 50 | 0 | 25 | 0 | 40 | 15 | 50 | 60 | 15 | 10 | 25 | 0 | 75 | 60 | 0 | 0 | 0 | 0 | 0 | 0 | 0 | 0 | 0 |
| Sedge, Umbrella | 75 | 75 | 50 | 60 | 75 | 45 | 75 | 0 | 70 | 55 | — | 45 | 20 | 50 | 65 | 0 | 80 | 40 | 15 | 0 | 0 | 50 | 0 | 20 | 0 | 50 | 30 |
| | 62 g ai/ha | | | | | | | | | | | | | | | | | | | | | | | | | | |
| | Compounds | | | | | | | | | | | | | | | | | | | | | | | | | | |
| | 45 | 47 | 48 | 49 | 51 | 55 | 56 | 58 | 62 | 63 | 64 | 65 | 67 | 75 | 76 | 79 | 80 | 81 | 88 | 95 | 96 | 101 | 102 | 106 | 108 | 112 | 115 |
| Barnyardgrass | 25 | 0 | 0 | 45 | 0 | 0 | 0 | 0 | 5 | 0 | 0 | 0 | 0 | 0 | 50 | 10 | 0 | 0 | 0 | 20 | 45 | 0 | 10 | 0 | 30 | 0 | 30 |
| Ducksalad | 0 | 0 | 20 | 0 | 0 | 0 | 15 | 0 | 25 | 0 | 0 | 15 | 30 | 0 | 45 | 0 | 20 | 0 | 0 | 10 | 45 | 0 | 20 | 0 | 50 | 0 | 30 |
| Rice | 30 | 0 | 0 | 15 | 0 | 0 | 0 | 0 | 15 | 0 | 0 | 0 | 0 | 0 | 75 | 20 | 20 | 20 | 0 | 10 | 40 | 0 | 20 | 0 | 0 | 0 | 55 |
| Sedge, Umbrella | 55 | 30 | 60 | 45 | 45 | 0 | 45 | 0 | 45 | 0 | 0 | 0 | 50 | 70 | 80 | 75 | 70 | 70 | 0 | 85 | 90 | 20 | 50 | 0 | 60 | 0 | 80 |
| | 62 g ai/ha | | | | | | | | | | | | | | | | | | | | | | | | | | |
| | Compounds | | | | | | | | | | | | | | | | | | | | | | | | | | |
| | 117 | 118 | 122 | 126 | 127 | 128 | 131 | 134 | 136 | 138 | 139 | 140 | 141 | 142 | 144 | 146 | 147 | 152 | 155 | 158 | 159 | 160 | 161 | 162 | 181 | 183 | 186 |
| Barnyardgrass | 10 | 0 | 20 | 0 | 20 | 30 | 0 | 10 | 20 | 0 | 15 | 0 | 0 | 50 | 20 | 0 | 0 | 40 | 0 | 0 | 30 | 20 | 0 | 45 | 0 | 0 | 20 |
| Ducksalad | 0 | 0 | 50 | 0 | 0 | 80 | 0 | 45 | 0 | 0 | 0 | 0 | 0 | 75 | 20 | 0 | 0 | 85 | 45 | 0 | 80 | 75 | 30 | 70 | 0 | 0 | 15 |
| Rice | 0 | 20 | 0 | 0 | 20 | 25 | 0 | 35 | 30 | 5 | 0 | 0 | 0 | 70 | 0 | 0 | 0 | 50 | 0 | 0 | 60 | 60 | 0 | 55 | 0 | 0 | 40 |
| Sedge, Umbrella | 40 | 0 | 50 | 0 | 80 | 80 | 55 | 55 | 65 | 0 | 75 | 0 | 0 | 75 | 80 | 60 | 30 | 80 | 0 | 15 | 10 | 50 | 0 | 70 | 10 | 0 | 80 |
| | 62 g ai/ha | | | | | | | | | | | | | | | | | | | | | | | | | | |
| | Compounds | | | | | | | | | | | | | | | | | | | | | | | | | | |
| | 187 | 188 | 192 | 193 | 194 | 207 | 209 | 210 | 215 | 216 | 219 | 222 | 223 | 225 | 226 | 227 | 228 | 229 | 231 | 232 | 235 | 236 | 241 | 242 | 243 | 244 | 245 |
| Barnyardgrass | 0 | 35 | 0 | 30 | 0 | 45 | 0 | 50 | 0 | 0 | 0 | 15 | 0 | 35 | 0 | 0 | 80 | 40 | 40 | 0 | 0 | 0 | 0 | 15 | 0 | 0 | 0 |
| Ducksalad | 20 | 20 | 0 | 55 | 0 | 55 | 65 | 40 | 40 | 0 | — | 70 | 0 | 10 | 0 | 0 | 80 | 0 | 10 | 0 | 0 | 0 | 0 | 65 | 75 | 25 | 60 |
| Rice | 15 | 35 | 0 | 25 | 0 | 30 | 0 | 35 | 0 | 0 | 0 | 30 | 0 | 55 | 0 | 0 | 75 | 35 | 30 | 0 | 0 | 0 | 0 | 50 | 15 | 40 | 25 |
| Sedge, Umbrella | 20 | 30 | 70 | 75 | 0 | 70 | 55 | 75 | 40 | 20 | 0 | 65 | 0 | 65 | 10 | 0 | 80 | 30 | 10 | 0 | 0 | 0 | 0 | 45 | 75 | 20 | 55 |
| | 62 g ai/ha | | | | | | | | | | | | | | | | | | | | | | | | | | |
| | Compounds | | | | | | | | | | | | | | | | | | | | | | | | | | |
| | 247 | 252 | 255 | 256 | 258 | 259 | 260 | 263 | 266 | 268 | 269 | 272 | 273 | 274 | 276 | 278 | 281 | 283 | 285 | 286 | 287 | 290 | 293 | 294 | 295 | 296 | 298 |
| Barnyardgrass | 0 | 0 | 0 | 0 | 0 | 0 | 0 | 0 | 0 | 65 | 45 | 0 | 20 | 0 | 0 | 0 | 0 | 0 | 10 | 40 | 0 | 0 | 0 | 75 | 0 | 15 | 15 |
| Ducksalad | 0 | 0 | 0 | 0 | 15 | 0 | 0 | 0 | 0 | 60 | 55 | 10 | 20 | 0 | 0 | 0 | 0 | 0 | 45 | 50 | 0 | 0 | 0 | 90 | 0 | 0 | 35 |
| Rice | 0 | 0 | 0 | 0 | 0 | 0 | 0 | 0 | 0 | 75 | 45 | 0 | 10 | 0 | 0 | 0 | 0 | 0 | 25 | 45 | 0 | 0 | 0 | 70 | 5 | 20 | 45 |
| Sedge, Umbrella | 0 | 0 | 0 | 0 | 0 | 0 | 0 | 0 | 0 | 55 | 45 | 30 | 70 | 0 | 45 | 0 | 15 | 0 | 25 | 30 | 0 | 0 | 20 | 80 | 80 | 75 | 0 |
| | 62 g ai/ha | | | | | | | | | | | | | | | | | | | | | | | | | | |
| | Compounds | | | | | | | | | | | | | | | | | | | | | | | | | | |
| | 299 | 300 | 301 | 302 | 303 | 304 | 305 | 306 | 307 | 308 | 309 | 310 | 313 | 319 | 331 | 332 | 333 | 334 | 335 | 336 | 340 | 341 | 342 | 343 | 344 | 349 | 352 |
| Barnyardgrass | 85 | 35 | 50 | 0 | 30 | 40 | 70 | 0 | 30 | 0 | 50 | 35 | 0 | 0 | 0 | 5 | 0 | 70 | 10 | 0 | 20 | 30 | 20 | 20 | 0 | 45 | 25 |
| Ducksalad | 95 | 75 | 85 | 0 | 55 | 65 | 0 | 50 | 60 | 0 | 85 | 45 | 0 | 0 | 0 | 20 | 0 | 75 | 0 | 45 | 50 | 20 | 50 | 40 | 0 | 80 | 20 |
| Rice | 70 | 40 | 50 | 0 | 30 | 45 | 75 | 30 | 55 | 15 | 60 | 35 | 0 | 0 | 0 | 0 | 0 | 70 | 25 | 10 | 20 | 20 | 20 | 20 | 0 | 50 | 20 |
| Sedge, Umbrella | 85 | 80 | 80 | 15 | 75 | 75 | 85 | 85 | 40 | 0 | 80 | 35 | 30 | 20 | 0 | 65 | 0 | 80 | 45 | 25 | 50 | 20 | 40 | 20 | 0 | 80 | 50 |
| | 62 g ai/ha | | | | | | | | | | | | | | | | | | | | | | | | | | |
| | Compounds | | | | | | | | | | | | | | | | | | | | | | | | | | |
| | 353 | 357 | 358 | 359 | 368 | 369 | 372 | 376 | 378 | 380 | 382 | 383 | 384 | 385 | 386 | 388 | 391 | 392 | 393 | 394 | 395 | 396 | 399 | 404 | 405 | 406 | 407 |
| Barnyardgrass | 0 | 0 | 0 | 15 | 0 | 10 | 65 | 45 | 0 | 35 | 10 | 0 | 65 | 20 | 30 | 0 | 55 | 15 | 45 | 0 | 75 | 65 | 50 | 0 | 0 | 10 | 0 |
| Ducksalad | 0 | 30 | 0 | 40 | 0 | 80 | 65 | 25 | 0 | 25 | 25 | 0 | 80 | 0 | 25 | 0 | 85 | 45 | 75 | 0 | 15 | 0 | 75 | 10 | 0 | 20 | 20 |
| Rice | 0 | 0 | 20 | 20 | 20 | 55 | 80 | 70 | 0 | 45 | 20 | 0 | 60 | 40 | 25 | 15 | 65 | 50 | 45 | 0 | 70 | 60 | 65 | 0 | 0 | 20 | 0 |
| Sedge, Umbrella | 0 | 40 | 15 | 80 | 20 | 75 | 80 | 80 | 0 | 80 | 75 | 0 | 80 | 75 | 65 | 65 | 80 | 70 | 70 | 0 | 70 | 70 | 65 | 70 | 40 | 75 | 20 |
| | | | | | | | | | | | | | | | | 62 g ai/ha | | | | | | 31 g ai/ha | | | 16 g ai/ha | | |
| | | | | | | | | | | | | | | | | Compounds | | | | | | Compounds | | | Compounds | | |
| | | | | | | | | | | | | | | | | 408 | 409 | 410 | 411 | 412 | 413 | 48 | 147 | 313 | 48 | 147 | 313 |
| | | | | | | | | | | | | | Barnyardgrass | | | 20 | 20 | 0 | 15 | 0 | 0 | 0 | 0 | 0 | 0 | 0 | 0 |
| | | | | | | | | | | | | | Ducksalad | | | 65 | 30 | 30 | 25 | 0 | 15 | 0 | 0 | 0 | 0 | 0 | 0 |
| | | | | | | | | | | | | | Rice | | | 25 | 20 | 20 | 0 | 15 | 35 | 0 | 0 | 0 | 0 | 0 | 0 |
| | | | | | | | | | | | | | Sedge, Umbrella | | | 60 | 70 | 70 | 60 | 70 | 10 | 0 | 20 | 0 | 0 | 0 | 0 |
| | | | | | | | | | | | | | Postemergence | | | | | | | | | | | | | | |
| | 500 g ai/ha | | | | | | | | | | | | | | | | | | | | | | | | | | |
| | Compounds | | | | | | | | | | | | | | | | | | | | | | | | | | |
| | 28 | 31 | 34 | 46 | 47 | 50 | 58 | 82 | 84 | 85 | 86 | 96 | 108 | 112 | 115 | 130 | 137 | 144 | 146 | 147 | 212 | 272 | 301 | 304 | 318 | 332 | 333 |
| Barley, Winter | | | | | | | | | | | | | | | — | — | 30 | 40 | — | — | — | 35 | 40 | 45 | — | 40 | — |
| Bermudagrass | 40 | 40 | 50 | — | 60 | 40 | 10 | 10 | — | 70 | 100 | 5 | 80 | 30 | 100 | 90 | — | — | — | — | 50 | 100 | — | — | 80 | 70 | 5 |
| Blackgrass | | | | | | | | | | | | | | | — | — | 60 | 40 | — | — | — | 65 | 45 | 95 | — | 70 | 55 |
| Bromegrass, Downy | | | | | | | | | | | | | | | — | — | 40 | 40 | — | — | — | 65 | 40 | 50 | — | 65 | 45 |
| Canarygrass | | | | | | | | | | | | | | | — | — | 40 | 50 | — | — | — | 55 | 50 | 45 | — | 70 | 45 |
| Chickweed | 100 | — | 100 | 100 | 100 | 0 | 85 | 100 | 70 | 100 | 100 | 100 | — | 100 | 100 | 100 | — | 100 | — | 100 | 100 | 100 | — | — | 80 | 100 | — |
| Cocklebur | 80 | 60 | 70 | 90 | 90 | 10 | 50 | 20 | 0 | 100 | 100 | 95 | 100 | 70 | 100 | 100 | — | 70 | 20 | 20 | 70 | 95 | — | — | 100 | 15 | — |
| Corn | 60 | 50 | 70 | 80 | 80 | 85 | 15 | 45 | 0 | 45 | 30 | 45 | 60 | 30 | 40 | 50 | — | 30 | 30 | 15 | 40 | 20 | — | — | 25 | 5 | 5 |
| Crabgrass, Large | 60 | 70 | 60 | 90 | 90 | 0 | 30 | 60 | 50 | 40 | 40 | 5 | 100 | 30 | 90 | 90 | — | 100 | 85 | 60 | 20 | 95 | — | — | 50 | 70 | 10 |
| Cupgrass, Woolly | — | 60 | 90 | 70 | 80 | 0 | 20 | 80 | 0 | 85 | 85 | 50 | 40 | 70 | 70 | 80 | — | 50 | 30 | 30 | 40 | 65 | — | — | 20 | 10 | 10 |
| Foxtail, Giant | 40 | 40 | 40 | 70 | 70 | 0 | 40 | 30 | 0 | 70 | 70 | 60 | 70 | 50 | 80 | 30 | — | 70 | 10 | 45 | 60 | 75 | — | — | 10 | 20 | 10 |
| Foxtail, Green | | | | | | | | | | | | | | | — | — | 50 | 60 | — | — | — | 60 | 65 | 60 | — | 70 | 65 |
| Goosegrass | 50 | 40 | 40 | 80 | 80 | — | 30 | 10 | 10 | 60 | 85 | 5 | 70 | 30 | 70 | 60 | — | 20 | 0 | 5 | 30 | 80 | — | — | 5 | 60 | 15 |
| Johnsongrass | 70 | 30 | 80 | 90 | 90 | 0 | 10 | 10 | 0 | 80 | 30 | 50 | 90 | 60 | 100 | 40 | — | — | 0 | 10 | 35 | 55 | — | — | 0 | 5 | 0 |
| Kochia | 80 | 70 | 90 | 100 | 100 | 10 | 85 | 70 | 80 | 90 | 95 | 90 | 90 | 80 | 90 | 85 | — | 85 | — | 80 | 90 | 100 | — | — | 60 | 100 | 100 |
| Lambsquarters | 100 | 90 | 100 | 100 | 100 | 0 | 90 | 100 | 50 | 100 | 100 | 100 | 100 | 100 | 100 | 100 | — | 100 | 100 | 100 | 100 | 100 | — | — | 100 | 100 | 100 |
| Morningglory | 60 | 70 | 40 | 80 | 90 | 10 | 20 | 50 | 0 | 80 | 85 | 50 | — | 100 | 100 | 100 | — | 80 | 0 | 0 | 70 | 100 | — | — | 0 | — | 10 |
| Nutsedge, Yellow | 20 | 30 | 20 | 40 | 30 | — | 5 | 5 | 0 | 40 | 30 | 5 | 30 | 20 | 30 | 0 | — | 0 | 0 | 0 | 20 | 0 | — | — | 0 | 0 | 0 |
| Oat, Wild | | | | | | | | | | | | | | | — | — | 50 | 50 | — | — | — | 65 | 60 | 70 | — | 70 | 55 |
| Pigweed | 100 | 100 | 100 | 100 | 100 | 100 | 85 | 100 | 100 | 100 | 100 | 100 | 100 | 100 | 100 | 100 | — | 100 | 100 | 100 | 90 | 100 | — | — | 100 | 95 | 100 |
| Ragweed | 50 | 70 | 40 | 80 | 90 | 85 | 85 | 30 | 0 | 60 | 70 | 85 | — | 70 | 90 | 85 | — | 80 | 0 | 10 | — | 95 | — | — | 0 | 60 | 50 |
| Ryegrass, Italian | | | | | | | | | | | | | | | — | — | 60 | 50 | — | — | — | 60 | 65 | 50 | — | 70 | 55 |
| Soybean | 80 | 80 | 80 | 100 | 100 | 100 | 90 | 40 | 20 | 95 | 95 | 100 | 60 | 60 | 90 | 70 | — | 90 | 80 | 70 | 85 | 95 | — | — | 90 | 90 | 45 |
| Surinam Grass | 60 | 50 | 70 | 50 | 70 | — | 25 | 40 | 10 | 80 | 85 | 60 | 50 | 60 | 60 | 60 | — | 40 | 20 | 60 | 50 | 60 | — | — | 10 | 30 | 10 |
| Velvetleaf | 80 | 70 | 70 | 80 | 90 | 100 | 60 | 75 | 0 | 85 | 85 | 95 | 90 | 90 | 90 | 85 | — | 50 | 40 | 40 | 70 | 100 | — | — | 60 | 90 | 80 |
| Wheat | | | | | | | | | | | | | | | — | — | 40 | 30 | — | — | — | 35 | 40 | 40 | — | 35 | 55 |
| Windgrass | | | | | | | | | | | | | | | — | — | 60 | 70 | — | — | — | 85 | 40 | 45 | — | 75 | 80 |
| | 500 g ai/ha | | | | | | | | | | | | | 250 g ai/ha | | | | | | | | | | | | | |
| | Compounds | | | | | | | | | | | | | Compounds | | | | | | | | | | | | | |
| | 338 | 353 | 376 | 391 | 392 | 408 | 411 | 412 | 414 | 415 | 417 | 429 | 431 | 27 | 28 | 31 | 34 | 44 | 46 | 47 | 50 | 58 | 60 | 62 | 80 | 81 | 82 |
| Barley, Winter | 40 | 25 | 40 | 30 | 35 | 40 | 40 | 50 | 45 | 45 | 35 | 40 | 60 | 10 | — | — | — | — | — | — | — | — | — | 10 | — | — | — |
| Bermudagrass | 5 | | | | | | | | | | | | | 30 | 30 | 20 | 50 | — | — | — | 30 | 10 | 5 | — | 100 | 20 | 0 |
| Blackgrass | 65 | 70 | 65 | 65 | 65 | 70 | 70 | 65 | 60 | 65 | 60 | 65 | 60 | 20 | — | — | — | — | — | — | — | — | — | 40 | — | — | — |
| Bromegrass, Downy | 45 | 35 | 50 | 65 | 60 | 55 | 45 | 60 | 60 | 65 | 65 | 55 | 60 | 20 | — | — | — | — | — | — | — | — | — | 30 | — | — | — |
| Canarygrass | 50 | 20 | 65 | 65 | 65 | 45 | 65 | 60 | 60 | 65 | 55 | 60 | 60 | 30 | — | — | — | — | — | — | — | — | — | 40 | — | — | — |
| Chickweed | — | | | | | | | | | | | | | 100 | 100 | 100 | 60 | 90 | 80 | 90 | 0 | 50 | 10 | — | 100 | 100 | 100 |
| Cocklebur | 5 | | | | | | | | | | | | | 80 | 60 | 40 | 50 | 70 | 70 | 70 | 10 | 20 | 10 | — | 100 | 60 | 5 |
| Corn | 10 | | | | | | | | | | | | | 50 | 40 | 40 | 50 | 80 | 80 | 80 | 85 | 5 | 0 | — | 70 | 40 | 10 |
| Crabgrass, Large | 15 | | | | | | | | | | | | | 40 | 40 | 60 | 60 | 90 | 90 | — | 0 | 15 | 5 | — | 90 | 30 | 0 |
| Cupgrass, Woolly | 0 | | | | | | | | | | | | | 70 | 40 | 40 | 80 | 60 | 70 | 70 | 0 | 20 | 5 | — | 90 | 5 | 80 |
| Foxtail, Giant | 10 | | | | | | | | | | | | | 40 | 30 | 30 | 40 | 50 | 60 | 60 | 0 | 20 | 5 | — | 80 | 20 | 10 |
| Foxtail, Green | 50 | 30 | 65 | 65 | 70 | 80 | 70 | 65 | 65 | 60 | 65 | 65 | 65 | 40 | — | — | — | — | — | — | — | — | — | 50 | — | — | — |
| Goosegrass | 10 | | | | | | | | | | | | | 50 | 50 | 40 | 40 | 50 | 50 | 60 | 90 | 10 | 5 | — | 80 | 10 | 10 |
| Johnsongrass | 0 | | | | | | | | | | | | | 40 | 60 | 30 | 70 | 40 | — | 90 | 0 | 5 | 0 | — | 90 | 5 | 5 |
| Kochia | 90 | | | | | | | | | | | | | 100 | 80 | 70 | 90 | 90 | 100 | 100 | 10 | 80 | 20 | — | 100 | 70 | 50 |
| Lambsquarters | 75 | | | | | | | | | | | | | 100 | 90 | 90 | 100 | 90 | 100 | 100 | 0 | 90 | 70 | — | 100 | 5 | 80 |
| Morningglory | 50 | | | | | | | | | | | | | 40 | 40 | 60 | 10 | 80 | 60 | 70 | 0 | 10 | 10 | — | 90 | 10 | 50 |
| Nutsedge, Yellow | 0 | | | | | | | | | | | | | 20 | 20 | 30 | 0 | 20 | 20 | 30 | — | 5 | 0 | — | 40 | 0 | 5 |
| Oat, Wild | 60 | 60 | 60 | 65 | 60 | 70 | 65 | 70 | 55 | 55 | 60 | 65 | 65 | 20 | — | — | — | — | — | — | — | — | — | 50 | — | — | — |
| Pigweed | 80 | | | | | | | | | | | | | 100 | 100 | 100 | 100 | 100 | 100 | 100 | 100 | 70 | 60 | — | 100 | 40 | 100 |
| Ragweed | 10 | | | | | | | | | | | | | 60 | 40 | 40 | 40 | 90 | 80 | 80 | 85 | 5 | 5 | — | 70 | 60 | 10 |
| Ryegrass, Italian | 60 | 20 | 65 | 65 | 65 | 70 | 65 | 60 | 60 | 45 | 60 | 60 | 55 | 20 | — | — | — | — | — | — | — | — | — | 40 | — | — | — |
| Soybean | 60 | | | | | | | | | | | | | 90 | 80 | 70 | 80 | 100 | 100 | 100 | 100 | 80 | 50 | — | 100 | 30 | 40 |
| Surinam Grass | 30 | | | | | | | | | | | | | 50 | 60 | 50 | 50 | 40 | 50 | 60 | 50 | — | 5 | — | 80 | 80 | 20 |
| Velvetleaf | 80 | | | | | | | | | | | | | 70 | 70 | 70 | 70 | — | 80 | 80 | 100 | 35 | 30 | — | 100 | 60 | 70 |
| Wheat | 45 | 35 | 25 | 35 | 35 | 40 | 55 | 50 | 30 | 30 | 35 | 45 | 45 | 20 | — | — | — | — | — | — | — | — | — | 10 | — | — | — |
| Windgrass | — | 10 | 65 | 70 | 65 | 65 | 65 | 65 | 65 | 65 | 55 | 60 | 60 | 30 | — | — | — | — | — | — | — | — | — | 50 | — | — | — |
| | 250 g ai/ha | | | | | | | | | | | | | | | | | | | | | | | | | | |
| | Compounds | | | | | | | | | | | | | | | | | | | | | | | | | | |
| | 84 | 85 | 86 | 96 | 108 | 112 | 114 | 115 | 130 | 137 | 139 | 144 | 146 | 147 | 212 | 268 | 272 | 295 | 296 | 301 | 304 | 318 | 332 | 333 | 334 | 338 | 353 |
| Barley, Winter | — | — | — | — | — | — | — | — | — | 20 | — | 30 | — | — | — | 30 | 35 | 35 | 45 | 30 | 35 | — | 40 | 55 | 30 | 35 | 20 |
| Bermudagrass | 0 | — | 30 | 5 | 60 | 30 | 90 | 60 | 90 | — | 0 | — | 0 | 70 | 10 | — | 100 | 0 | — | — | — | — | 50 | 5 | — | 0 | — |
| Blackgrass | — | — | — | — | — | — | — | — | — | 40 | — | 40 | — | — | — | 60 | 45 | 65 | 75 | 90 | 70 | — | 100 | 50 | 95 | 50 | 60 |
| Bromegrass, Downy | — | — | — | — | — | — | — | — | — | 20 | — | 40 | — | — | — | 40 | 60 | 45 | 45 | 40 | 40 | — | 50 | 45 | 45 | 40 | 30 |
| Canarygrass | — | — | — | — | — | — | — | — | — | 40 | — | 40 | — | — | — | 50 | 40 | 35 | 55 | 50 | 45 | — | 60 | 40 | 45 | 40 | 10 |
| Chickweed | 70 | 100 | 100 | 100 | — | 100 | 100 | 100 | 100 | — | 80 | 100 | 100 | 90 | 80 | — | 100 | 100 | — | — | — | 10 | 100 | — | — | — | — |
| Cocklebur | 0 | 100 | 100 | 90 | 100 | 60 | 30 | 100 | 90 | — | 10 | 70 | 10 | 5 | 70 | — | 60 | 10 | — | — | — | 10 | 10 | 80 | — | 5 | — |
| Corn | 0 | 35 | 20 | 35 | 50 | — | 40 | — | 45 | — | 10 | 20 | 10 | 10 | 40 | — | 15 | 15 | — | — | — | 10 | 5 | 5 | — | 10 | — |
| Crabgrass, Large | 0 | 10 | 10 | 5 | 70 | 30 | 100 | 70 | 20 | — | 0 | 100 | 50 | 10 | 20 | — | 80 | 35 | — | — | — | 30 | 50 | 5 | — | 10 | — |
| Cupgrass, Woolly | 0 | 80 | 80 | 30 | 40 | 60 | 70 | 70 | 60 | — | 5 | 50 | 30 | 10 | 30 | — | 60 | 15 | — | — | — | 10 | 10 | 5 | — | 0 | — |
| Foxtail, Giant | 0 | 50 | 50 | 45 | — | 50 | 10 | 60 | 15 | — | 10 | 50 | 0 | 0 | 50 | — | 60 | 40 | — | — | — | 5 | 15 | 10 | — | 10 | — |
| Foxtail, Green | — | — | — | — | — | — | — | — | — | 50 | — | 60 | — | — | — | 50 | 45 | 60 | 60 | 60 | 50 | — | 50 | 50 | 45 | 40 | 20 |
| Goosegrass | 5 | 60 | 80 | 5 | 70 | 30 | 70 | 70 | 60 | — | 5 | 10 | 0 | 5 | 25 | — | 75 | 50 | — | — | — | 5 | 15 | 10 | — | 10 | — |
| Johnsongrass | 0 | 20 | 10 | 50 | 70 | 50 | 70 | 80 | 30 | — | 0 | 10 | 0 | 10 | 35 | — | 45 | 5 | — | — | — | 0 | 5 | 0 | — | 0 | — |
| Kochia | 10 | 90 | 95 | 85 | 90 | — | 100 | 90 | 70 | — | 50 | 70 | 20 | 50 | 90 | — | 100 | 90 | — | — | — | 60 | 95 | 100 | — | 90 | — |
| Lambsquarters | 0 | 50 | 100 | 95 | 100 | 100 | 100 | 100 | 100 | — | — | 100 | 100 | 100 | 90 | — | 100 | 50 | — | — | — | 100 | 100 | 80 | — | 75 | — |
| Morningglory | 0 | 60 | 85 | 30 | — | 70 | — | — | 80 | — | 60 | 20 | 0 | 0 | 70 | — | 100 | 30 | — | — | — | 0 | 80 | 10 | — | 40 | — |
| Nutsedge, Yellow | 0 | 20 | 10 | 5 | 30 | 20 | 10 | 30 | 0 | — | 0 | 0 | 0 | 0 | 20 | — | 0 | 5 | — | — | — | 0 | 0 | 0 | — | 0 | — |
| Oat, Wild | — | — | — | — | — | — | — | — | — | 40 | — | 50 | — | — | — | 65 | 55 | 50 | 60 | 75 | 60 | — | 70 | 60 | 95 | 50 | 40 |
| Pigweed | 100 | 100 | 100 | 100 | 100 | 100 | 100 | 100 | 100 | — | 80 | 100 | 100 | 100 | 70 | — | 100 | 95 | — | — | — | 100 | 95 | 100 | — | 80 | — |
| Ragweed | 0 | 40 | 70 | 80 | — | 70 | 90 | 70 | 85 | — | 45 | 50 | 0 | 10 | 80 | — | 80 | 40 | — | — | — | 0 | 40 | 50 | — | 5 | — |
| Ryegrass, Italian | — | — | — | — | — | — | — | — | — | 40 | — | 40 | — | — | — | 40 | 45 | 65 | 65 | 45 | 50 | — | 70 | 50 | 45 | 50 | 15 |
| Soybean | 20 | 80 | 90 | 100 | 60 | 50 | 70 | 80 | 40 | — | 35 | 90 | 30 | 40 | 70 | — | 85 | 55 | — | — | — | 80 | 60 | 40 | — | 35 | — |
| Surinam Grass | 0 | 80 | 70 | 40 | 40 | 40 | 20 | 60 | 30 | — | 0 | 40 | 0 | 30 | 20 | — | 35 | 15 | — | — | — | 0 | 10 | 5 | — | 0 | — |
| Velvetleaf | 0 | 80 | 80 | 70 | 80 | 80 | 80 | 90 | 40 | — | 50 | 50 | 40 | — | 50 | — | 100 | 70 | — | — | — | 40 | 85 | 50 | — | 40 | — |
| Wheat | — | — | — | — | — | — | — | — | — | 30 | — | 30 | — | — | — | 25 | 35 | 45 | 40 | 55 | 40 | — | 35 | 50 | 75 | 35 | 30 |
| Windgrass | — | — | — | — | — | — | — | — | — | 50 | — | 60 | — | — | — | 100 | — | 65 | 65 | 35 | 40 | — | 65 | 50 | 75 | — | 10 |
| | 250 g ai/ha | | | | | | | | | | | | | | | | 125 g ai/ha | | | | | | | | | | |
| | Compounds | | | | | | | | | | | | | | | | Compounds | | | | | | | | | | |
| | 375 | 376 | 380 | 384 | 391 | 392 | 393 | 408 | 411 | 412 | 414 | 415 | 417 | 429 | 430 | 431 | 27 | 28 | 31 | 33 | 34 | 35 | 44 | 46 | 47 | 50 | 58 |
| Barley, Winter | 40 | 30 | 30 | 35 | 30 | 35 | 35 | 35 | 35 | 40 | 45 | 35 | 30 | 35 | 45 | 45 | 10 | — | — | — | — | — | — | — | — | — | — |
| Bermudagrass | 60 | — | — | — | — | — | — | — | — | — | — | — | — | — | | | 30 | 10 | 20 | 30 | 40 | 20 | 90 | 100 | — | 30 | 5 |
| Blackgrass | 55 | 50 | 55 | 45 | 55 | 45 | 65 | 65 | 65 | 65 | 45 | 60 | 60 | 60 | 65 | 60 | 20 | — | — | — | — | — | — | — | — | — | — |
| Bromegrass, Downy | 60 | 40 | 65 | 55 | 55 | 55 | 55 | 55 | 45 | 55 | 55 | 60 | 50 | 45 | 55 | 55 | 20 | — | — | — | — | — | — | — | — | — | — |
| Canarygrass | 35 | 55 | 45 | 40 | 60 | 50 | 65 | 40 | 65 | 60 | 50 | 55 | 45 | 55 | 60 | 60 | 30 | — | — | — | — | — | — | — | — | — | — |
| Chickweed | 100 | — | — | — | — | — | — | — | — | — | — | — | — | — | | | 100 | 100 | 90 | 100 | 50 | 90 | 90 | 70 | 90 | 0 | 20 |
| Cocklebur | 15 | — | — | — | — | — | — | — | — | — | — | — | — | — | | | 70 | 60 | 40 | 70 | 20 | 70 | 70 | 70 | 70 | 10 | 20 |
| Corn | 20 | — | — | — | — | — | — | — | — | — | — | — | — | — | | | 40 | 40 | 40 | 40 | 30 | 40 | 70 | 80 | 70 | 80 | 5 |
| Crabgrass, Large | 20 | — | — | — | — | — | — | — | — | — | — | — | — | — | | | 30 | 30 | 60 | 50 | 40 | 40 | 90 | 90 | — | 0 | 5 |
| Cupgrass, Woolly | 45 | — | — | — | — | — | — | — | — | — | — | — | — | — | | | 60 | 30 | 30 | 60 | 60 | 60 | 60 | 60 | 60 | 0 | 5 |
| Foxtail, Giant | 60 | — | — | — | — | — | — | — | — | — | — | — | — | — | | | 40 | 20 | 30 | 30 | 40 | 40 | 30 | 60 | 60 | 0 | 5 |
| Foxtail, Green | 65 | 60 | 60 | 65 | 60 | 65 | 65 | 70 | 65 | 65 | 65 | 50 | 60 | 55 | 60 | 60 | 40 | — | — | — | — | — | — | — | — | — | — |
| Goosegrass | 50 | — | — | — | — | — | — | — | — | — | — | — | — | — | | | 30 | 30 | 30 | 40 | 40 | 50 | 40 | 50 | 60 | 30 | 5 |
| Johnsongrass | 10 | — | — | — | — | — | — | — | — | — | — | — | — | — | | | 40 | 60 | 20 | 90 | 70 | 70 | 30 | 60 | 30 | 0 | 0 |
| Kochia | 90 | — | — | — | — | — | — | — | — | — | — | — | — | — | | | 60 | 40 | 70 | 90 | 90 | 90 | 90 | — | 100 | 10 | 80 |
| Lambsquarters | 40 | — | — | — | — | — | — | — | — | — | — | — | — | — | | | 80 | 70 | 80 | 90 | 80 | 90 | 90 | 100 | 100 | 0 | 70 |
| Morningglory | 20 | — | — | — | — | — | — | — | — | — | — | — | — | — | | | 40 | 0 | 50 | 30 | 10 | 40 | 50 | 60 | 50 | 0 | 10 |
| Nutsedge, Yellow | | | | | | | | | | | | | | | | | 20 | 0 | 0 | 10 | 0 | 0 | 20 | 20 | 20 | — | 5 |
| Oat, Wild | 65 | 55 | 65 | 55 | 45 | 60 | 65 | 65 | 60 | 65 | 50 | 45 | 60 | 60 | 65 | 60 | 20 | — | — | — | — | — | — | — | — | — | — |
| Pigweed | 85 | — | — | — | — | — | — | — | — | — | — | — | — | — | | | 100 | 90 | 90 | 100 | 100 | 100 | 100 | 100 | 100 | 100 | 60 |
| Ragweed | 50 | — | — | — | — | — | — | — | — | — | — | — | — | — | | | 50 | 40 | 40 | 70 | 30 | 50 | 50 | 70 | 80 | 85 | 5 |
| Ryegrass, Italian | 75 | 60 | 65 | 60 | 55 | 60 | 65 | 60 | 65 | 55 | 60 | 40 | 55 | 55 | 60 | 45 | 10 | — | — | — | — | — | — | — | — | — | — |
| Soybean | | | | | | | | | | | | | | | | | 80 | 80 | 50 | 90 | 80 | 90 | 100 | 100 | — | 100 | 75 |
| Surinam Grass | 50 | — | — | — | — | — | — | — | — | — | — | — | — | — | | | 30 | 20 | 40 | 50 | 30 | 60 | 40 | 40 | 30 | 50 | — |
| Velvetleaf | 90 | — | — | — | — | — | — | — | — | — | — | — | — | — | | | 60 | 60 | 60 | — | 70 | 80 | 80 | 80 | 80 | 100 | 10 |
| Wheat | 40 | 20 | 30 | 30 | 30 | 35 | 40 | 35 | 45 | 45 | 25 | 30 | 35 | 40 | 55 | 40 | 20 | — | — | — | — | — | — | — | — | — | — |
| Windgrass | 70 | 40 | 65 | 60 | 65 | 55 | 60 | 55 | 65 | 55 | 60 | 55 | 55 | 45 | 60 | 55 | 30 | — | — | — | — | — | — | — | — | — | — |
| | 125 g ai/ha | | | | | | | | | | | | | | | | | | | | | | | | | | |
| | Compounds | | | | | | | | | | | | | | | | | | | | | | | | | | |
| | 60 | 62 | 80 | 81 | 82 | 84 | 85 | 86 | 96 | 108 | 112 | 114 | 115 | 130 | 137 | 139 | 144 | 146 | 147 | 212 | 268 | 272 | 295 | 296 | 301 | 304 | 318 |
| Barley, Winter | — | 10 | — | — | — | — | — | — | — | — | — | — | — | — | 20 | — | 20 | — | — | — | 15 | 35 | 35 | 40 | 30 | 35 | — |
| Bermudagrass | 0 | — | 100 | 0 | 0 | — | 65 | — | 0 | 30 | 0 | 90 | 60 | 85 | — | 0 | 0 | — | — | 0 | — | 80 | 0 | — | — | — | — |
| Blackgrass | — | 40 | — | — | — | — | — | — | — | — | — | — | — | — | 40 | — | 30 | — | — | — | 45 | 45 | 65 | 65 | 75 | 50 | — |
| Bromegrass, Downy | — | 20 | — | — | — | — | — | — | — | — | — | — | — | — | 20 | — | 40 | — | — | — | 30 | 55 | 45 | 35 | 40 | 40 | — |
| Canarygrass | — | 30 | — | — | — | — | — | — | — | — | — | — | — | — | 30 | — | 40 | — | — | — | 30 | 35 | 35 | 40 | 45 | 40 | — |
| Chickweed | 10 | — | 100 | 95 | 95 | 50 | 100 | 100 | 100 | — | 10 | — | 100 | 100 | — | 70 | 100 | 80 | 30 | 80 | — | 100 | 100 | — | — | — | 0 |
| Cocklebur | 10 | — | 90 | 55 | 5 | 0 | 80 | 95 | 10 | 80 | 30 | 30 | 90 | 30 | — | 10 | 40 | 5 | 5 | 60 | — | 60 | 5 | — | — | — | 5 |
| Corn | 0 | — | 70 | 40 | 10 | 0 | 30 | 20 | 30 | 50 | 20 | 30 | 40 | 45 | — | 10 | 20 | 5 | 5 | 35 | — | 15 | 10 | — | — | — | 10 |
| Crabgrass, Large | 0 | — | — | 10 | 0 | 0 | 5 | — | 0 | 70 | 10 | 100 | 70 | — | — | 0 | 10 | 0 | 0 | 10 | — | 80 | 30 | — | — | — | 0 |
| Cupgrass, Woolly | 5 | — | 90 | 5 | 60 | 0 | 50 | 70 | 30 | 40 | 30 | 70 | 70 | 50 | — | 0 | 30 | 10 | 0 | 5 | — | 40 | 15 | — | — | — | 0 |
| Foxtail, Giant | 5 | — | 80 | 10 | 0 | 0 | 20 | 20 | 20 | 50 | 50 | — | 40 | 10 | — | 0 | 10 | 0 | 0 | 20 | — | 45 | 35 | — | — | — | 0 |
| Foxtail, Green | — | 50 | — | — | — | — | — | — | — | — | — | — | — | — | 50 | — | 60 | — | — | — | 45 | 40 | 50 | 50 | 45 | 45 | — |
| Goosegrass | 0 | — | 80 | 5 | 10 | 0 | 40 | 60 | 5 | 40 | 20 | 60 | 60 | 20 | — | 0 | 5 | 0 | 0 | 10 | — | 65 | 20 | — | — | — | 0 |
| Johnsongrass | 0 | — | 90 | 0 | 0 | 0 | 10 | — | 50 | 50 | 20 | 70 | 50 | 10 | — | 0 | 0 | 0 | — | 30 | — | 40 | 5 | — | — | — | 0 |
| Kochia | 20 | — | 100 | 20 | 40 | 0 | 60 | 85 | 80 | 80 | 20 | 80 | 90 | 30 | — | 50 | 65 | — | 10 | 80 | — | 100 | 85 | — | — | — | 10 |
| Lambsquarters | 30 | — | 100 | 5 | 50 | 0 | 30 | 100 | 80 | 100 | 20 | 90 | 100 | 100 | — | — | 100 | 80 | 100 | 90 | — | 100 | 5 | — | — | — | 70 |
| Morningglory | 10 | — | 90 | 10 | 10 | 0 | 40 | 75 | 5 | 60 | 20 | 30 | 80 | 40 | — | 50 | 0 | 0 | 0 | 10 | — | 100 | 10 | — | — | — | 0 |
| Nutsedge, Yellow | 0 | — | 40 | 0 | 5 | 0 | 20 | 5 | 0 | 30 | 0 | 0 | 10 | 0 | — | 0 | 0 | 0 | 0 | 10 | — | 0 | 5 | — | — | — | 0 |
| Oat, Wild | — | 40 | — | — | — | — | — | — | — | — | — | — | — | — | 40 | — | 50 | — | — | — | 35 | 45 | 50 | 60 | 65 | 60 | — |
| Pigweed | 40 | — | 100 | 10 | 100 | 100 | 100 | 100 | 100 | 100 | 60 | 100 | 100 | 100 | — | 65 | 100 | 90 | 90 | — | — | 100 | — | — | — | — | 100 |
| Ragweed | 5 | — | 70 | 60 | 0 | 0 | 10 | 65 | 50 | — | 0 | 80 | 70 | 40 | — | 45 | 0 | 0 | 0 | 60 | — | 65 | 30 | — | — | — | 0 |
| Ryegrass, Italian | — | 20 | — | — | — | — | — | — | — | — | — | — | — | — | 40 | — | 20 | — | — | — | 30 | 45 | 40 | 45 | 45 | 50 | — |
| Soybean | 40 | — | 90 | 30 | 30 | 0 | 60 | 70 | 100 | 60 | 0 | 70 | 70 | 40 | — | 30 | 85 | 20 | 35 | 60 | — | 80 | 50 | — | — | — | 70 |
| Surinam Grass | 5 | — | 80 | 50 | 10 | 0 | 30 | 60 | 40 | 40 | 40 | 10 | 40 | 10 | — | 0 | 0 | 0 | 0 | 5 | — | 10 | 5 | — | — | — | 0 |
| Velvetleaf | 20 | — | 100 | 55 | 10 | 0 | 75 | 60 | 70 | 60 | 0 | 70 | 90 | 30 | — | 50 | 20 | 35 | 30 | — | — | 95 | 65 | — | — | — | 35 |
| Wheat | — | 10 | — | — | — | — | — | — | — | — | — | — | — | — | 30 | — | 30 | — | — | — | 0 | 35 | 45 | 35 | 45 | 40 | — |
| Windgrass | — | 40 | — | — | — | — | — | — | — | — | — | — | — | — | 50 | — | 60 | — | — | — | 60 | 70 | 60 | 50 | 35 | 35 | — |
| | 125 g ai/ha | | | | | | | | | | | | | | | | | | | | | 62 g ai/ha | | | | | |
| | Compounds | | | | | | | | | | | | | | | | | | | | | Compounds | | | | | |
| | 332 | 333 | 334 | 338 | 353 | 375 | 376 | 380 | 384 | 391 | 392 | 393 | 408 | 411 | 412 | 414 | 415 | 417 | 429 | 430 | 431 | 27 | 28 | 31 | 33 | 34 | 35 |
| Barley, Winter | 35 | 45 | 30 | 30 | 15 | 30 | 20 | 20 | 35 | 10 | 35 | 35 | 30 | 35 | 35 | 30 | 35 | 20 | 35 | 35 | 40 | 10 | — | — | — | — | — |
| Bermudagrass | 10 | 0 | — | 0 | | 30 | — | — | — | — | — | — | — | — | — | — | — | — | — | | | 30 | 10 | 10 | 20 | 10 | 0 |
| Blackgrass | 70 | 40 | 95 | 45 | 20 | 45 | 40 | 45 | 45 | 45 | 45 | 60 | 50 | 65 | 45 | 40 | 60 | 50 | 60 | 55 | 55 | 10 | — | — | — | — | — |
| Bromegrass, Downy | 40 | — | 35 | 35 | 20 | 60 | 35 | 60 | 45 | 45 | 55 | 45 | 45 | 40 | 45 | 45 | 60 | 40 | 40 | 50 | 45 | 10 | — | — | — | — | — |
| Canarygrass | 45 | 35 | 35 | 25 | 10 | 35 | 50 | 40 | 35 | 40 | 45 | 65 | 30 | 60 | 45 | 45 | 50 | 40 | 40 | 50 | 45 | 20 | — | — | — | — | — |
| Chickweed | 100 | — | — | — | | 100 | — | — | — | — | — | — | — | — | — | — | — | — | — | | | 70 | 90 | 90 | 90 | — | 80 |
| Cocklebur | 10 | 5 | — | 0 | | 10 | — | — | — | — | — | — | — | — | — | — | — | — | — | | | 50 | 40 | 40 | 50 | 20 | 60 |
| Corn | 0 | 5 | — | 0 | | 15 | — | — | — | — | — | — | — | — | — | — | — | — | — | | | 30 | 20 | 40 | 40 | 20 | 30 |
| Crabgrass, Large | — | 5 | — | 5 | | 20 | — | — | — | — | — | — | — | — | — | — | — | — | — | | | 20 | 30 | 50 | — | 20 | 40 |
| Cupgrass, Woolly | 5 | 0 | — | 0 | | 30 | — | — | — | — | — | — | — | — | — | — | — | — | — | | | 60 | 30 | 30 | 60 | 40 | 40 |
| Foxtail, Giant | 10 | 5 | — | 5 | | 45 | — | — | — | — | — | — | — | — | — | — | — | — | — | | | 20 | 20 | 20 | 30 | 20 | 40 |
| Foxtail, Green | 40 | 40 | 35 | 40 | 20 | 65 | 60 | 60 | 60 | 55 | 60 | 65 | 60 | 65 | 60 | 60 | 50 | 55 | 50 | 50 | 60 | 30 | — | — | — | — | — |
| Goosegrass | 15 | 10 | — | 5 | | 15 | — | — | — | — | — | — | — | — | — | — | — | — | — | | | 30 | 20 | 30 | 40 | 30 | 40 |
| Johnsongrass | 5 | 0 | — | 0 | | 5 | — | — | — | — | — | — | — | — | — | — | — | — | — | | | 40 | 20 | 20 | 90 | 70 | 70 |
| Kochia | 90 | 100 | — | 60 | | 85 | — | — | — | — | — | — | — | — | — | — | — | — | — | | | 60 | 40 | 70 | 90 | 80 | 90 |
| Lambsquarters | 65 | 80 | — | 5 | | 40 | — | — | — | — | — | — | — | — | — | — | — | — | — | | | 70 | 70 | 80 | 90 | 40 | 90 |
| Morningglory | 10 | 0 | — | 0 | | | | | | | | | | | | | | | | | | 40 | 0 | 50 | 20 | 10 | 20 |
| Nutsedge, Yellow | 0 | 0 | — | 0 | | | | | | | | | | | | | | | | | | 20 | 0 | 0 | 0 | 0 | 0 |
| Oat, Wild | 65 | 60 | 75 | 45 | 30 | 55 | 45 | 60 | 45 | 40 | 40 | 65 | 55 | 60 | 65 | 45 | 45 | 40 | 60 | 60 | 55 | 20 | — | — | — | — | — |
| Pigweed | 90 | 100 | — | 5 | | | | | | | | | | | | | | | | | | 90 | 90 | 90 | 100 | 100 | 100 |
| Ragweed | 30 | 30 | — | 5 | | 10 | — | — | — | — | — | — | — | — | — | — | — | — | — | | | 30 | 30 | 30 | 60 | 20 | 50 |
| Ryegrass, Italian | 60 | 35 | 35 | 30 | 15 | 65 | 55 | 50 | 55 | 40 | 50 | 60 | 45 | 60 | 45 | 55 | 40 | 40 | 50 | 60 | 45 | 10 | — | — | — | — | — |
| Soybean | 50 | 30 | — | 30 | | | | | | | | | | | | | | | | | | 80 | 70 | 30 | 80 | 70 | 90 |
| Surinam Grass | 5 | 5 | — | 0 | | | | | | | | | | | | | | | | | | 20 | — | 30 | 50 | 20 | 40 |
| Velvetleaf | 60 | 50 | — | 0 | | 85 | — | — | — | — | — | — | — | — | — | — | — | — | — | | | 60 | 50 | 50 | 90 | 20 | 70 |
| Wheat | 30 | 50 | 75 | 25 | 15 | 30 | 20 | 25 | 20 | 20 | 30 | 40 | 30 | 35 | 35 | 10 | 25 | 20 | 35 | 50 | 40 | 10 | — | — | — | — | — |
| Windgrass | 60 | 25 | 60 | 15 | 10 | 60 | 40 | 60 | 45 | 65 | 55 | 55 | 45 | 55 | 45 | 60 | 50 | 40 | 40 | 45 | 40 | 20 | — | — | — | — | — |
| | 62 g ai/ha | | | | | | | | | | | | | | | | | | | | | | | | | | |
| | Compounds | | | | | | | | | | | | | | | | | | | | | | | | | | |
| | 44 | 46 | 47 | 50 | 58 | 60 | 62 | 80 | 81 | 82 | 84 | 85 | 86 | 96 | 108 | 112 | 114 | 115 | 137 | 139 | 144 | 146 | 147 | 212 | 268 | 272 | 295 |
| Barley, Winter | — | — | — | — | — | — | 10 | — | — | — | — | — | — | — | — | — | — | — | 10 | — | 0 | — | — | — | 0 | 35 | 35 |
| Bermudagrass | — | — | 60 | 0 | 0 | 0 | — | — | 0 | 0 | — | 0 | 10 | 0 | 30 | 0 | 60 | 10 | — | 0 | 0 | — | — | 0 | — | 50 | 0 |
| Blackgrass | — | — | — | — | — | — | 40 | — | — | — | — | — | — | — | — | — | — | — | 40 | — | 30 | — | — | — | 30 | 35 | 45 |
| Bromegrass, Downy | — | — | — | — | — | — | 20 | — | — | — | — | — | — | — | — | — | — | — | 10 | — | 20 | — | — | — | 10 | 30 | 45 |
| Canarygrass | — | — | — | — | — | — | 0 | — | — | — | — | — | — | — | — | — | — | — | 20 | — | 40 | — | — | — | 10 | 35 | 30 |
| Chickweed | 90 | 70 | 90 | 0 | 20 | 5 | — | 100 | 95 | 70 | 0 | 100 | 100 | 100 | — | 0 | 100 | 100 | — | 0 | 100 | 10 | 5 | 60 | — | 95 | 100 |
| Cocklebur | 50 | 60 | 50 | 10 | 15 | 10 | — | 90 | 55 | 5 | 0 | 20 | 85 | 5 | 70 | 0 | 30 | 90 | — | 5 | 10 | 5 | 0 | 10 | — | 15 | 5 |
| Corn | 70 | 80 | 70 | 20 | 0 | 0 | — | 70 | 40 | 10 | 0 | 30 | 15 | 30 | 50 | 10 | 30 | 40 | — | 0 | 10 | 5 | 0 | 15 | — | 10 | 5 |
| Crabgrass, Large | 60 | — | 90 | 0 | 5 | 0 | — | 90 | 5 | 0 | 0 | 0 | 10 | 0 | 10 | 0 | 20 | 60 | — | 0 | 0 | 0 | 0 | 0 | — | 40 | — |
| Cupgrass, Woolly | 50 | 40 | 60 | 0 | 5 | 0 | — | 80 | 5 | — | 0 | 40 | 50 | 5 | 20 | 0 | 70 | 50 | — | 0 | 0 | 10 | 0 | 5 | — | 25 | 10 |
| Foxtail, Giant | 30 | 40 | 60 | 0 | 0 | 0 | — | 70 | 10 | 0 | 0 | 10 | 10 | 10 | 30 | 30 | 10 | 40 | — | 0 | 0 | 0 | 0 | 10 | — | 15 | 30 |
| Foxtail, Green | — | — | — | — | — | — | 40 | — | — | — | — | — | — | — | — | — | — | — | 30 | — | 40 | — | — | — | 40 | 35 | 45 |
| Goosegrass | 40 | 40 | 50 | 30 | 0 | 0 | — | 80 | 0 | 0 | 0 | 35 | 50 | 5 | 20 | 20 | 60 | 40 | — | 0 | 5 | 0 | 0 | 10 | — | 20 | 20 |
| Johnsongrass | 30 | 60 | 30 | 0 | 0 | 0 | — | 80 | 0 | 0 | 0 | 10 | 10 | 0 | 20 | 20 | 70 | 30 | — | 0 | 0 | 0 | 0 | 0 | — | 10 | 5 |
| Kochia | 90 | 90 | 100 | 0 | 75 | 10 | — | 100 | 0 | 10 | 0 | 20 | 80 | 80 | 60 | 0 | 80 | 80 | — | 50 | — | 5 | 5 | 80 | — | 95 | 80 |
| Lambsquarters | 90 | 80 | 90 | 0 | 60 | 10 | — | 90 | 5 | 30 | 0 | 10 | 100 | 60 | 70 | 20 | 90 | 100 | — | — | 100 | 40 | 90 | 80 | — | 100 | 0 |
| Morningglory | 50 | 60 | 50 | 0 | 10 | 10 | — | 90 | 10 | 10 | 0 | 10 | 10 | 0 | 30 | 0 | 10 | 50 | — | 0 | 0 | 0 | 0 | 10 | — | 80 | 10 |
| Nutsedge, Yellow | 20 | 20 | 20 | — | 0 | 0 | — | 40 | 0 | 0 | 0 | 10 | 0 | 0 | 20 | 0 | 0 | 0 | — | 0 | 0 | 0 | 0 | 0 | — | 0 | 5 |
| Oat, Wild | — | — | — | — | — | — | 30 | — | — | — | — | — | — | — | — | — | — | — | 20 | — | 30 | — | — | — | 20 | 40 | 50 |
| Pigweed | 100 | 100 | 100 | 50 | 60 | 40 | — | 100 | 5 | 85 | 100 | 100 | 100 | 70 | 60 | 10 | 100 | 100 | — | 50 | 100 | 80 | 90 | 50 | — | 100 | 80 |
| Ragweed | 50 | 70 | 70 | 80 | 0 | 0 | — | 70 | 40 | 0 | 0 | 10 | 10 | 50 | — | 0 | 60 | 70 | — | 0 | 0 | 0 | 0 | 60 | — | 60 | 25 |
| Ryegrass, Italian | — | — | — | — | — | — | 20 | — | — | — | — | — | — | — | — | — | — | — | 20 | — | 20 | — | — | — | 10 | 30 | 40 |
| Soybean | 100 | 100 | 100 | 100 | 15 | 20 | — | 90 | 10 | 20 | 0 | 55 | 45 | 100 | 50 | 0 | 70 | 60 | — | 20 | 70 | 20 | 10 | 50 | — | 60 | 50 |
| Surinam Grass | 40 | 40 | 30 | 10 | — | 5 | — | 80 | 50 | 10 | 0 | 25 | 10 | 0 | 20 | 20 | 10 | 30 | — | 0 | 0 | 0 | 0 | 0 | — | 5 | 5 |
| Velvetleaf | 60 | 50 | 80 | 100 | 10 | 20 | — | 100 | 55 | 5 | 0 | 60 | 40 | 40 | 40 | 0 | 60 | 60 | — | 30 | 0 | 30 | 0 | 0 | — | 85 | 60 |
| Wheat | — | — | — | — | — | — | 10 | — | — | — | — | — | — | — | — | — | — | — | 30 | — | 20 | — | — | — | 0 | 30 | 45 |
| Windgrass | — | — | — | — | — | — | 30 | — | — | — | — | — | — | — | — | — | — | — | 30 | — | 50 | — | — | — | 30 | 60 | 50 |
| | 62 g ai/ha | | | | | | | | | | | | | | | | | | | | | | | | | 31 g ai/ha | |
| | Compounds | | | | | | | | | | | | | | | | | | | | | | | | | Compounds | |
| | 296 | 301 | 304 | 318 | 332 | 333 | 334 | 338 | 353 | 375 | 376 | 380 | 384 | 391 | 392 | 393 | 408 | 411 | 412 | 414 | 415 | 417 | 429 | 430 | 431 | 27 | 33 |
| Barley, Winter | 35 | 30 | 35 | — | 35 | 40 | 20 | 20 | 5 | 20 | 20 | 20 | 10 | 10 | 35 | 35 | 30 | 30 | 30 | 10 | 35 | 20 | 25 | 30 | 40 | 0 | — |
| Bermudagrass | — | — | — | — | 5 | 0 | — | 0 | — | 20 | — | — | — | — | — | — | — | — | — | — | — | — | — | | | 20 | 20 |
| Blackgrass | 60 | 55 | 60 | — | 60 | 30 | 90 | 40 | 0 | 35 | 40 | 40 | 45 | 40 | 35 | 60 | 40 | 50 | 45 | 40 | 50 | 35 | 45 | 50 | 45 | 10 | — |
| Bromegrass, Downy | 35 | 40 | 40 | — | 35 | 40 | 30 | 30 | 10 | 60 | 35 | 55 | 40 | 45 | 35 | 40 | 40 | 40 | 40 | 40 | 60 | 35 | 40 | 45 | 40 | 10 | — |
| Canarygrass | 30 | 35 | 40 | — | 35 | 35 | 25 | 20 | 0 | 30 | 45 | 40 | 30 | 40 | 40 | 65 | 20 | 60 | 45 | 45 | 45 | 30 | 20 | 40 | 40 | 10 | — |
| Chickweed | — | — | — | 0 | 60 | — | — | — | — | 100 | — | — | — | — | — | — | — | — | — | — | — | — | — | | | 70 | 40 |
| Cocklebur | — | — | — | 0 | 5 | 0 | — | 0 | — | 10 | — | — | — | — | — | — | — | — | — | — | — | — | — | | | 40 | — |
| Corn | — | — | — | 0 | 0 | 5 | — | 0 | — | 10 | — | — | — | — | — | — | — | — | — | — | — | — | — | | | 30 | 40 |
| Crabgrass, Large | — | — | — | 0 | 30 | 0 | — | 0 | — | 5 | — | — | — | — | — | — | — | — | — | — | — | — | — | | | 20 | 40 |
| Cupgrass, Woolly | — | — | — | 0 | 5 | 0 | — | 0 | — | 30 | — | — | — | — | — | — | — | — | — | — | — | — | — | | | 0 | 50 |
| Foxtail, Giant | — | — | — | 0 | 10 | 5 | — | 5 | — | 40 | — | — | — | — | — | — | — | — | — | — | — | — | — | | | 20 | 30 |
| Foxtail, Green | 40 | 40 | 40 | — | 35 | 30 | 35 | 30 | 15 | 50 | 40 | 60 | 60 | 55 | 60 | 60 | 40 | 60 | 45 | 60 | 45 | 50 | 40 | 45 | 45 | 30 | — |
| Goosegrass | — | — | — | 0 | 10 | 5 | — | 0 | — | 15 | — | — | — | — | — | — | — | — | — | — | — | — | — | | | 30 | 40 |
| Johnsongrass | — | — | — | 0 | 0 | 0 | — | 0 | — | 5 | — | — | — | — | — | — | — | — | — | — | — | — | — | | | 30 | 70 |
| Kochia | — | — | — | 0 | 80 | 90 | — | 50 | — | 70 | — | — | — | — | — | — | — | — | — | — | — | — | — | | | 50 | 60 |
| Lambsquarters | — | — | — | 60 | 50 | 60 | — | 0 | — | 10 | — | — | — | — | — | — | — | — | — | — | — | — | — | | | 60 | 90 |
| Morningglory | — | — | — | 0 | 5 | 0 | — | 0 | — | 10 | — | — | — | — | — | — | — | — | — | — | — | — | — | | | 30 | 20 |
| Nutsedge, Yellow | — | — | — | 0 | 0 | 0 | — | 0 | — | | | | | | | | | | | | | | | | | 20 | 0 |
| Oat, Wild | 35 | 45 | 60 | — | 50 | 50 | 55 | 40 | 10 | 45 | 40 | 55 | 35 | 30 | 30 | 55 | 45 | 60 | 60 | 35 | 40 | 35 | 45 | 55 | 45 | 20 | — |
| Pigweed | — | — | — | 100 | 85 | 65 | — | 0 | — | 80 | — | — | — | — | — | — | — | — | — | — | — | — | — | | | 80 | 90 |
| Ragweed | — | — | — | 0 | 5 | 5 | — | 0 | — | 5 | — | — | — | — | — | — | — | — | — | — | — | — | — | | | 30 | — |
| Ryegrass, Italian | 35 | 35 | 50 | — | 60 | 35 | 30 | 25 | 10 | 65 | 45 | 45 | 45 | 35 | 35 | 55 | 40 | 45 | 40 | 50 | 30 | 35 | 45 | 55 | 40 | 10 | — |
| Soybean | — | — | — | 65 | 45 | 30 | — | 10 | — | | | | | | | | | | | | | | | | | 80 | 80 |
| Surinam Grass | — | — | — | 0 | 5 | 5 | — | 0 | — | 20 | — | — | — | — | — | — | — | — | — | — | — | — | — | | | 20 | 50 |
| Velvetleaf | — | — | — | 0 | 40 | — | — | 0 | — | 60 | — | — | — | — | — | — | — | — | — | — | — | — | — | | | 50 | 60 |
| Wheat | 35 | 40 | 35 | — | 30 | 40 | 65 | 25 | 10 | 25 | 20 | 20 | 10 | 0 | 20 | 35 | 30 | 35 | 35 | 10 | 20 | 20 | 25 | 45 | 40 | 10 | — |
| Windgrass | 45 | 35 | 35 | — | 35 | 20 | 60 | 10 | 0 | 40 | 40 | 45 | 45 | 60 | 35 | 45 | 40 | 40 | 40 | 55 | 50 | 40 | 40 | 35 | 40 | 20 | — |

| | 31 g ai/ha | | | | | | | | | | | | | | | | 16 g ai/ha | |
|---|---|---|---|---|---|---|---|---|---|---|---|---|---|---|---|---|---|---|
| | Compounds | | | | | | | | | | | | | | | | Compounds | |
| | 35 | 60 | 62 | 80 | 81 | 114 | 139 | 268 | 295 | 296 | 334 | 375 | 380 | 384 | 393 | 430 | 33 | 35 |
| Barley, Winter | — | — | 0 | — | — | — | — | 0 | 35 | 30 | 20 | 10 | 10 | 0 | 20 | 25 | | |
| Bermudagrass | — | 0 | — | — | 0 | 20 | 0 | — | 0 | — | — | 5 | — | — | — | — | 0 | 0 |
| Blackgrass | — | — | 20 | — | — | — | — | 0 | 45 | 45 | 75 | 30 | 40 | 45 | 55 | 45 | | |
| Bromegrass, Downy | — | — | 10 | — | — | — | — | 0 | 45 | 25 | 20 | 40 | 45 | 30 | 40 | 45 | | |
| Canarygrass | — | — | 0 | — | — | — | — | 10 | 25 | 30 | 20 | 30 | 30 | 20 | 60 | 30 | | |
| Chickweed | 70 | 5 | — | 90 | 90 | 80 | 0 | — | 85 | — | — | 100 | — | — | — | — | 30 | 70 |
| Cocklebur | 20 | 10 | — | 70 | 50 | 30 | 5 | — | 5 | — | — | 0 | — | — | — | — | 20 | 20 |
| Corn | 30 | 0 | — | 70 | 15 | 30 | 0 | — | 5 | — | — | 5 | — | — | — | — | 40 | 30 |
| Crabgrass, Large | 0 | 0 | — | 90 | 5 | 20 | 0 | — | 20 | — | — | 5 | — | — | — | — | — | 0 |
| Cupgrass, Woolly | 30 | 0 | — | 80 | 5 | 40 | 0 | — | 10 | — | — | 5 | — | — | — | — | 30 | 20 |
| Foxtail, Giant | 10 | 0 | — | 70 | 10 | 0 | 0 | — | 30 | — | — | 10 | — | — | — | — | 30 | 0 |
| Foxtail, Green | — | — | 40 | — | — | — | — | 35 | 40 | 40 | 30 | 40 | 45 | 60 | 60 | 40 | | |
| Goosegrass | 30 | 0 | — | 80 | 0 | 50 | 0 | — | 5 | — | — | 10 | — | — | — | — | 30 | 0 |
| Johnsongrass | 60 | 0 | — | 80 | 0 | 70 | 0 | — | 0 | — | — | 0 | — | — | — | — | 70 | 50 |
| Kochia | — | 5 | — | 100 | 0 | 30 | 20 | — | 50 | — | — | 60 | — | — | — | — | 60 | 20 |
| Lambsquarters | 70 | 10 | — | 90 | 0 | 80 | — | — | 0 | — | — | 5 | — | — | — | — | 70 | 50 |
| Morningglory | 10 | 10 | — | 80 | 5 | 0 | 0 | — | 5 | — | — | 5 | — | — | — | — | 0 | 0 |
| Nutsedge, Yellow | 0 | 0 | — | 40 | 0 | 0 | 0 | — | 0 | — | — | | | | | | 0 | 0 |
| Oat, Wild | — | — | 20 | — | — | — | — | 20 | 45 | 35 | 40 | 40 | 40 | 30 | 45 | 45 | | |
| Pigweed | 100 | 20 | — | 100 | 0 | 90 | 50 | — | 60 | — | — | 5 | — | — | — | — | 90 | 70 |
| Ragweed | 20 | 0 | — | 70 | 0 | 60 | 0 | — | 20 | — | — | 0 | — | — | — | — | 50 | 20 |
| Ryegrass, Italian | — | — | 0 | — | — | — | — | 0 | 40 | 25 | 20 | 60 | 30 | 35 | 50 | 45 | | |
| Soybean | 90 | 20 | — | 90 | 5 | 20 | 20 | — | 35 | — | — | | | | | | 70 | 60 |
| Surinam Grass | 10 | 0 | — | 80 | 50 | 10 | 0 | — | 5 | — | — | 10 | — | — | — | — | 20 | 0 |
| Velvetleaf | 50 | 15 | — | 100 | 5 | 30 | 0 | — | 60 | — | — | 0 | — | — | — | — | 40 | 40 |
| Wheat | — | — | 0 | — | — | — | — | 0 | 40 | 30 | 35 | 20 | 10 | 0 | 35 | 35 | | |
| Windgrass | — | — | 0 | — | — | — | — | 20 | 50 | 45 | 60 | 40 | 40 | 40 | 40 | 30 | | |

Preemergence

| | 500 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 1 | 2 | 3 | 6 | 7 | 9 | 10 | 11 | 13 | 14 | 16 | 28 | 30 | 31 | 34 | 46 | 47 | 48 | 50 | 55 | 56 | 57 | 58 | 61 | 63 | 64 | 65 |
| Bermudagrass | 100 | 95 | 95 | 90 | 100 | 90 | 100 | 90 | 100 | 100 | 100 | 100 | 90 | 100 | 100 | 100 | 100 | 95 | 10 | 100 | 95 | 100 | 100 | 95 | 100 | 90 | 95 |
| Cocklebur | 100 | 80 | 40 | 100 | — | 100 | — | — | 20 | 90 | 75 | 30 | 20 | — | 0 | 90 | 100 | 10 | 80 | 0 | 0 | 0 | 30 | 0 | 0 | 0 | 0 |
| Corn | 80 | 60 | 35 | 80 | 80 | 65 | 60 | 80 | 75 | 95 | 80 | 0 | — | — | 0 | 10 | 10 | 0 | 10 | 0 | 0 | 0 | 0 | 0 | 30 | 30 | 20 |
| Crabgrass, Large | 100 | 100 | 95 | 100 | 100 | 95 | 100 | 100 | 100 | 100 | 100 | 100 | 100 | 100 | 100 | 100 | 100 | 90 | 80 | 100 | 100 | 100 | 95 | 100 | 100 | 90 | 100 |
| Cupgrass, Woolly | 65 | 80 | 70 | 75 | — | 30 | 85 | 70 | 70 | 100 | 85 | 20 | 60 | 60 | 70 | 80 | 80 | 20 | 70 | 65 | 40 | 70 | 80 | 10 | 5 | 10 | 10 |
| Foxtail, Giant | 80 | 100 | 100 | 100 | 100 | 75 | 100 | 90 | 100 | 100 | 100 | 100 | 90 | 100 | 100 | 100 | 90 | 50 | 10 | 100 | 100 | 100 | 100 | 60 | 80 | 80 | 60 |
| Goosegrass | 95 | 100 | 95 | 100 | 90 | 85 | 85 | 90 | 100 | 100 | 95 | 100 | 80 | 90 | 100 | 100 | 100 | 90 | 100 | 95 | 95 | 90 | 95 | 95 | 90 | 70 | 80 |
| Johnsongrass | 90 | 100 | 100 | 100 | 100 | 80 | 100 | 95 | 100 | 100 | 100 | 100 | 90 | 90 | 100 | 100 | 100 | 85 | 0 | 100 | 90 | 85 | 100 | 60 | 100 | 60 | 100 |
| Kochia | 100 | 100 | 100 | 100 | 100 | 100 | 100 | 100 | 100 | 100 | 100 | 100 | 100 | 100 | 100 | 100 | 40 | 100 | 100 | 100 | 95 | 100 | 100 | 100 | 90 | 60 | 90 |
| Lambsquarters | 100 | 100 | 100 | 100 | 100 | 100 | 100 | 100 | 100 | 100 | 100 | 100 | 100 | 100 | 100 | 100 | 100 | 95 | 100 | 100 | 100 | 100 | 100 | 100 | 100 | 90 | 100 |
| Morningglory | 100 | 100 | 60 | 70 | 0 | 35 | 60 | 15 | 100 | 100 | 70 | 60 | 80 | 40 | 100 | 90 | 90 | 20 | 0 | 75 | 55 | 25 | 80 | 100 | 40 | 0 | 10 |
| Nightshade | 100 | 100 | 95 | 100 | 100 | 100 | 100 | 100 | 100 | 100 | 100 | 100 | 100 | 100 | 100 | 100 | 100 | 100 | 95 | 95 | 95 | 100 | 100 | 90 | 95 | 95 | 100 |
| Nutsedge, Yellow | 80 | — | 70 | 85 | 60 | 60 | 60 | 60 | 75 | 90 | 90 | 20 | — | 0 | 0 | 0 | 0 | 30 | 80 | 10 | 20 | 20 | 50 | 30 | 40 | 0 | 0 |
| Pigweed | 100 | 100 | 95 | 100 | 100 | 100 | 100 | 100 | 100 | 100 | 100 | 100 | 100 | 100 | 100 | 100 | 100 | 100 | 95 | 95 | 100 | 100 | 100 | 95 | 100 | 100 | 100 |
| Ragweed | 100 | 100 | 100 | 100 | 100 | 100 | 100 | 100 | 100 | 100 | 100 | 90 | 20 | 80 | 40 | 90 | 100 | 100 | 100 | 80 | 95 | 100 | 100 | 80 | 95 | 20 | 60 |
| Soybean | 65 | 100 | 85 | 100 | 80 | 55 | 70 | 90 | 75 | 95 | 95 | 80 | 30 | 20 | 0 | 10 | 20 | 30 | 0 | 20 | 15 | 60 | 40 | 10 | 70 | 60 | 20 |
| Sunflower | 35 | 0 | 0 | 100 | 0 | 20 | 40 | 10 | 80 | 100 | 90 | 0 | 30 | 20 | 0 | 10 | 40 | 0 | 0 | 0 | 0 | 0 | 0 | 0 | 30 | 0 | 0 |
| Surinam Grass | 100 | 100 | 100 | 100 | 80 | 80 | 90 | 100 | 100 | 100 | 100 | 90 | 50 | 60 | 70 | 80 | 80 | 60 | 85 | 100 | 75 | 75 | 100 | 5 | 60 | 50 | 50 |
| Velvetleaf | 100 | 100 | 100 | 100 | 100 | 100 | 80 | 100 | 100 | 100 | 100 | 100 | 90 | 100 | 100 | 100 | 100 | 70 | 5 | 100 | 100 | 100 | 100 | 100 | 100 | 60 | 60 |
| | 500 g ai/ha | | | | | | | | | | | | | | | | | | | | | | | | | | |
| | Compounds | | | | | | | | | | | | | | | | | | | | | | | | | | |
| | 88 | 96 | 102 | 108 | 115 | 130 | 134 | 137 | 140 | 144 | 145 | 146 | 147 | 149 | 152 | 153 | 156 | 162 | 182 | 188 | 193 | 195 | 198 | 199 | 202 | 205 | 207 |
| Bermudagrass | 95 | 100 | 100 | 100 | 100 | 100 | 100 | 100 | 85 | 90 | 90 | 90 | 95 | 95 | 100 | 100 | 95 | 40 | 95 | 100 | 100 | 65 | 100 | 100 | 100 | 100 | 100 |
| Cocklebur | 0 | 90 | 70 | 50 | 100 | 100 | 0 | 0 | 0 | 80 | 0 | 0 | 0 | 0 | 100 | 0 | 100 | 75 | 0 | 0 | 100 | 0 | 10 | 0 | 20 | 0 | 0 |
| Corn | 60 | 40 | 30 | 50 | 30 | 10 | 60 | 5 | 0 | 50 | 20 | 30 | 60 | 10 | 70 | 0 | 50 | 75 | 0 | 5 | 100 | 0 | 0 | 0 | 10 | 40 | 20 |
| Crabgrass, Large | 100 | 100 | 100 | 100 | 100 | 95 | 100 | 100 | 100 | 100 | 80 | 70 | 100 | 100 | 100 | 80 | 100 | 100 | 100 | 100 | 100 | 70 | 95 | 95 | 95 | 100 | 100 |
| Cupgrass, Woolly | 90 | 70 | 40 | 80 | 90 | 70 | 45 | 40 | 45 | 70 | 40 | 0 | 60 | 70 | 100 | 60 | 90 | 100 | 0 | 75 | 70 | 10 | 70 | 70 | 60 | 50 | 40 |
| Foxtail, Giant | 100 | 100 | 80 | 100 | 100 | 95 | 85 | 85 | 85 | 100 | 70 | 80 | 100 | 100 | 100 | 0 | 95 | 75 | 60 | 100 | 100 | 20 | 100 | 100 | 100 | 95 | 100 |
| Goosegrass | 100 | 95 | 100 | 100 | 100 | 50 | 95 | 100 | 75 | 80 | 80 | 60 | 80 | 90 | 100 | 95 | 95 | 80 | 80 | 90 | 95 | 100 | 100 | 95 | 95 | 95 | 100 |
| Johnsongrass | 70 | 95 | 100 | 100 | 100 | 80 | 65 | 40 | 75 | 90 | 90 | 70 | 80 | 100 | 100 | 85 | 100 | 95 | 75 | 75 | 100 | 0 | 95 | 90 | 75 | 85 | 50 |
| Kochia | 100 | 100 | 60 | 100 | 100 | 100 | 100 | 100 | 100 | 100 | 80 | 100 | 100 | 90 | 100 | 100 | 100 | 100 | 90 | 95 | 100 | 85 | 100 | 90 | 95 | 90 | 95 |
| Lambsquarters | 100 | — | 100 | 100 | 100 | 80 | 100 | 100 | 100 | 100 | 100 | 100 | 100 | 95 | 100 | 100 | 100 | 100 | 95 | 100 | 100 | 95 | 100 | 100 | 100 | 100 | 95 |
| Morningglory | 20 | 100 | 80 | 90 | 80 | 30 | 75 | 60 | 0 | 90 | 30 | 50 | 0 | 60 | 100 | 15 | 100 | 100 | 100 | 75 | 100 | 0 | 85 | 70 | 85 | 50 | 0 |
| Nightshade | 95 | 100 | 100 | 100 | 100 | 60 | 100 | 90 | 95 | 100 | 90 | 100 | 100 | 95 | 100 | 100 | 100 | 100 | 95 | 100 | 100 | 0 | 100 | 100 | 100 | 100 | 100 |
| Nutsedge, Yellow | 0 | 10 | 30 | 80 | 20 | 0 | 0 | 0 | 0 | 40 | 0 | 0 | 0 | 0 | 70 | 45 | 85 | 60 | 0 | 0 | 60 | 70 | 5 | 0 | 0 | 10 | 20 |
| Pigweed | 100 | 100 | 100 | 100 | 100 | 100 | 100 | 100 | 100 | 100 | 95 | 100 | 100 | 100 | 100 | 100 | 100 | 100 | 95 | 100 | 100 | 90 | 100 | 100 | 100 | 100 | 100 |
| Ragweed | 20 | 100 | 100 | 90 | 100 | 80 | 100 | 100 | 95 | 80 | 20 | 70 | 70 | 5 | 100 | 100 | 100 | 100 | 65 | 100 | 100 | 90 | 100 | 80 | 95 | 100 | 100 |
| Soybean | 10 | 50 | 20 | 10 | 0 | 10 | 35 | 10 | 0 | 80 | 60 | 0 | 5 | 0 | 100 | 15 | 100 | 100 | 15 | 0 | 100 | 0 | 25 | 30 | 30 | 60 | 0 |
| Sunflower | 0 | 35 | 20 | 20 | 10 | 0 | 0 | 0 | 0 | 40 | 30 | 0 | 0 | 0 | 70 | 0 | 85 | 90 | 0 | 0 | 50 | 10 | 20 | 0 | 10 | 0 | 0 |
| Surinam Grass | 95 | 65 | 50 | 90 | 90 | 60 | 75 | 60 | 70 | 70 | 70 | 50 | 60 | 85 | 100 | 100 | 95 | 95 | 85 | 80 | 100 | 85 | 100 | 75 | 60 | 85 | 10 |
| Velvetleaf | 50 | 100 | 60 | 100 | 100 | 100 | 100 | 100 | 20 | 100 | 100 | 100 | 90 | 60 | 100 | 100 | 100 | 100 | 100 | 75 | 100 | 100 | 100 | 85 | 100 | 100 | 60 |
| | 500 g ai/ha | | | | | | | | | | | | | | | | | | | | | | | | | | |
| | Compounds | | | | | | | | | | | | | | | | | | | | | | | | | | |
| | 210 | 212 | 213 | 215 | 221 | 222 | 223 | 242 | 244 | 268 | 269 | 287 | 293 | 294 | 298 | 299 | 301 | 304 | 305 | 306 | 307 | 309 | 310 | 314 | 318 | 332 | 333 |
| Bermudagrass | 100 | 100 | 90 | 100 | 85 | 100 | 100 | 100 | 100 | 100 | 100 | 95 | 100 | 90 | 95 | 100 | 95 | 100 | 100 | 100 | 100 | 100 | 100 | 90 | 95 | 100 | 100 |
| Cocklebur | 5 | 95 | 100 | 60 | — | 100 | 0 | 50 | 0 | 85 | 50 | 60 | 60 | 85 | 0 | 95 | 60 | 0 | 90 | 5 | 75 | 60 | 30 | 20 | 0 | 0 | 5 |
| Corn | 40 | 40 | 40 | 50 | 10 | 100 | 50 | 25 | 45 | 70 | 55 | — | 95 | 80 | 30 | 60 | 45 | 30 | 45 | 50 | 45 | 60 | 10 | 0 | 40 | 5 | 0 |
| Crabgrass, Large | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 60 | 100 | 100 | 100 |
| Cupgrass, Woolly | 10 | 30 | 30 | 50 | 10 | 100 | 80 | 100 | 95 | 95 | 85 | 35 | 100 | 85 | 60 | 100 | 90 | 80 | 70 | 50 | 85 | 100 | — | 20 | 10 | 10 | 30 |
| Foxtail, Giant | 60 | 95 | 100 | 100 | 90 | 100 | 100 | 100 | 100 | 100 | 100 | 100 | 100 | 90 | 100 | 100 | 100 | 90 | 100 | 100 | 100 | 100 | 100 | 70 | 80 | 80 | 75 |
| Goosegrass | 95 | 100 | 90 | 100 | 50 | 100 | 100 | 100 | 100 | 100 | 100 | 100 | 100 | 100 | 100 | 100 | 100 | 100 | 95 | 100 | 100 | 100 | 95 | 85 | 90 | 100 | 100 |
| Johnsongrass | 70 | 60 | 80 | 85 | 80 | 100 | — | 100 | 100 | 100 | 100 | 95 | 100 | 100 | 90 | 100 | 95 | 80 | 95 | — | 100 | 100 | 80 | 90 | 95 | 80 | 5 |
| Kochia | 95 | 95 | 100 | 100 | 100 | 100 | 100 | — | — | 100 | 100 | 100 | 100 | 100 | 100 | 100 | 100 | 100 | 100 | 100 | 100 | 100 | 100 | 70 | 100 | 100 | 100 |
| Lambsquarters | 100 | 100 | 100 | 100 | 100 | 100 | 100 | — | — | 100 | 100 | 100 | 100 | 100 | 100 | 100 | 100 | 100 | 100 | 100 | 100 | 100 | 100 | 85 | 100 | 100 | 100 |
| Morningglory | 10 | 30 | 0 | 40 | 0 | 100 | 100 | 15 | 55 | 5 | 40 | — | — | 50 | 45 | 100 | 60 | 10 | 10 | 20 | 15 | 40 | 10 | 40 | 85 | 0 | 10 |
| Nightshade | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 95 | 100 | 100 |
| Nutsedge, Yellow | 30 | 0 | 0 | 0 | 0 | 60 | 0 | 25 | 0 | 70 | 60 | 85 | 20 | 90 | 30 | 80 | 20 | 5 | 50 | 40 | 50 | 60 | 15 | 0 | 0 | 0 | 0 |
| Pigweed | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 90 | 100 | 100 | 100 |
| Ragweed | 90 | 95 | 0 | 100 | 60 | 100 | 100 | 100 | 100 | 100 | 90 | 100 | 100 | 100 | 0 | 100 | 70 | 70 | 100 | 100 | 100 | 100 | 80 | 60 | 70 | 70 | 65 |
| Soybean | 0 | 60 | 0 | 0 | 20 | 100 | 90 | 75 | 15 | 95 | 60 | 95 | 100 | 100 | 60 | 95 | 45 | 20 | 100 | 40 | 100 | 80 | — | 40 | 40 | 10 | 0 |
| Sunflower | 0 | 0 | 0 | 0 | 30 | 30 | 30 | 95 | 20 | 90 | 15 | 100 | — | 100 | 0 | 100 | 0 | 5 | 50 | 0 | 60 | 60 | 0 | 45 | 0 | 0 | 0 |
| Surinam Grass | 50 | 20 | 60 | 50 | 45 | 100 | 85 | 100 | 100 | 100 | 95 | 95 | 100 | 100 | 70 | 100 | 90 | 85 | 60 | 95 | 90 | 100 | 50 | 20 | 60 | 15 | 20 |
| Velvetleaf | 80 | 100 | 0 | 100 | 80 | 100 | 100 | 100 | 100 | 100 | 80 | 100 | 100 | 100 | 100 | 100 | 100 | 60 | 100 | 10 | 100 | 100 | 100 | 85 | 100 | 70 | 55 |
| | 500 g ai/ha | | | | | | | | | | | | | | | | | | | | | | | 250 g ai/ha | | | |
| | Compounds | | | | | | | | | | | | | | | | | | | | | | | Compounds | | | |
| | 336 | 340 | 341 | 342 | 343 | 352 | 372 | 376 | 382 | 385 | 386 | 391 | 392 | 393 | 395 | 396 | 399 | 409 | 414 | 417 | 418 | 419 | 420 | 1 | 2 | 3 | 6 |
| Bermudagrass | 100 | 100 | 100 | 90 | 90 | 100 | 100 | 100 | 90 | 100 | 95 | 95 | 95 | 100 | 100 | 100 | 100 | 85 | 100 | 100 | 100 | 100 | 100 | 90 | 95 | 90 | 90 |
| Cocklebur | 50 | 40 | 100 | 100 | 100 | 30 | 90 | 5 | 45 | 100 | 70 | 80 | 80 | 100 | 85 | 0 | 80 | 80 | 30 | 0 | 35 | 70 | 70 | — | 65 | 10 | 100 |
| Corn | 85 | 50 | 90 | 90 | 80 | 50 | 65 | 50 | 60 | — | 85 | 85 | 90 | 100 | 90 | 80 | 95 | 80 | 80 | 65 | 80 | 40 | 40 | 65 | 35 | 35 | 80 |
| Crabgrass, Large | 100 | 100 | 100 | 85 | 100 | 100 | 95 | 100 | 100 | 100 | 100 | 100 | 100 | 100 | 100 | 100 | 100 | 95 | 100 | 100 | 100 | 100 | 100 | 95 | 100 | 95 | 95 |
| Cupgrass, Woolly | 85 | 75 | 100 | 70 | 100 | 85 | 100 | 85 | 40 | 95 | 95 | 100 | 85 | 100 | 100 | 100 | 95 | 80 | 100 | 85 | 95 | 65 | 65 | — | 60 | 65 | 75 |
| Foxtail, Giant | 90 | 85 | 80 | 70 | 80 | 100 | 90 | 100 | 100 | 100 | 100 | 100 | 100 | 100 | 100 | 100 | 100 | 95 | 100 | 100 | 100 | 100 | 100 | 75 | 100 | 100 | 85 |
| Goosegrass | 100 | 90 | 90 | 90 | 85 | 100 | 90 | 100 | 100 | 100 | 95 | 95 | 98 | 100 | 100 | 100 | 100 | 95 | 100 | 100 | 100 | 100 | 100 | 90 | 95 | 90 | 100 |
| Johnsongrass | 100 | 90 | 100 | 100 | 100 | 100 | 90 | 100 | 100 | 100 | 100 | 100 | 100 | 100 | 100 | 100 | 100 | 95 | 100 | 100 | 95 | 100 | 100 | 80 | 100 | 90 | 90 |
| Kochia | 100 | 100 | 100 | 100 | 100 | 100 | 100 | 100 | 100 | 100 | 100 | 100 | 100 | 100 | 100 | 100 | 100 | 95 | 100 | 100 | 95 | 100 | 100 | 100 | 100 | 100 | 100 |
| Lambsquarters | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 90 | 100 |
| Morningglory | 100 | 50 | 50 | 100 | 100 | 60 | 80 | 5 | 100 | 100 | 100 | 100 | 100 | — | 100 | 70 | 75 | 100 | 95 | 95 | 100 | 100 | 100 | 15 | 45 | 25 | 30 |
| Nightshade | 100 | 100 | 100 | 85 | 95 | 100 | 100 | 100 | 100 | — | 100 | 95 | 100 | — | 100 | 100 | 100 | 95 | 100 | 95 | 100 | 100 | 100 | 100 | 100 | 95 | 100 |
| Nutsedge, Yellow | 60 | 60 | 60 | 60 | 90 | 50 | 85 | 60 | 60 | 100 | 60 | 90 | 90 | — | 90 | 80 | 95 | 55 | 20 | 0 | 0 | 0 | 25 | 60 | 90 | 65 | 80 |
| Pigweed | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 95 | 95 | 100 |
| Ragweed | 100 | 75 | 100 | 100 | 100 | 60 | 100 | 50 | 100 | 95 | 100 | 100 | 100 | 100 | 100 | 95 | 100 | 95 | 65 | 90 | 100 | 100 | 85 | 100 | 100 | 100 | 100 |
| Soybean | 100 | 10 | 100 | 100 | 100 | 70 | 90 | 10 | 100 | 100 | 95 | 100 | 95 | — | 100 | 100 | 100 | 100 | 65 | 40 | 90 | 95 | 85 | 25 | 100 | 40 | 100 |
| Sunflower | 45 | 5 | 100 | 100 | 100 | 0 | 60 | 0 | 20 | — | 85 | 100 | 100 | — | 55 | 15 | 35 | 55 | 0 | 0 | 0 | 95 | 65 | 25 | 0 | 0 | 100 |
| Surinam Grass | 100 | 85 | 100 | 100 | 100 | 100 | 100 | 100 | 100 | — | 95 | 95 | 90 | — | 100 | 100 | 100 | 95 | 100 | 95 | 100 | 100 | 95 | 100 | 100 | 100 | 100 |
| Velvetleaf | 100 | 100 | 100 | 100 | 100 | 100 | 100 | 50 | 100 | 100 | 100 | 100 | 100 | 100 | 100 | 100 | 100 | 100 | 100 | 100 | 100 | 100 | 100 | 85 | 100 | 85 | 100 |
| | 250 g ai/ha | | | | | | | | | | | | | | | | | | | | | | | | | | |
| | Compounds | | | | | | | | | | | | | | | | | | | | | | | | | | |
| | 7 | 9 | 10 | 11 | 13 | 14 | 16 | 27 | 28 | 29 | 30 | 31 | 34 | 41 | 44 | 46 | 47 | 48 | 50 | 55 | 56 | 57 | 58 | 60 | 61 | 62 | 63 |
| Bermudagrass | 85 | 85 | 100 | 50 | 90 | 85 | 95 | 100 | 100 | 100 | 90 | 100 | 90 | 100 | 20 | 100 | 100 | 90 | 0 | 100 | 95 | 95 | 100 | 100 | 80 | 95 | 90 |
| Cocklebur | 0 | 100 | — | 20 | 0 | 70 | 40 | 20 | 0 | 40 | 20 | 40 | 0 | 10 | 0 | 90 | 0 | 0 | 80 | 0 | — | 0 | 10 | 5 | — | 0 | 0 |
| Corn | 40 | 55 | 60 | 80 | 60 | 75 | 70 | 20 | 0 | 0 | 60 | — | 0 | 0 | 0 | 10 | 0 | 0 | 0 | 0 | 0 | 0 | 0 | 0 | 0 | 0 | 5 |
| Crabgrass, Large | 100 | 95 | 100 | 85 | 100 | 100 | 100 | 100 | 100 | 100 | 100 | 100 | 100 | 100 | 20 | 100 | 100 | 70 | 20 | 95 | 100 | 100 | 95 | 95 | 100 | 90 | 90 |
| Cupgrass, Woolly | 80 | 25 | 85 | 55 | 55 | 95 | 65 | 40 | 20 | 60 | 30 | 30 | 70 | 30 | 10 | 30 | 60 | 5 | 30 | 55 | 35 | 25 | 60 | 70 | 0 | 20 | 0 |
| Foxtail, Giant | 80 | 70 | 80 | 80 | 95 | 95 | 90 | 100 | 50 | 100 | 70 | 70 | 90 | 30 | 10 | 100 | 80 | 10 | 0 | 75 | 100 | 100 | 100 | 80 | 40 | 85 | 10 |
| Goosegrass | 90 | 85 | — | 80 | 100 | 100 | 90 | 100 | 90 | 90 | 50 | 40 | 100 | 40 | 50 | 100 | 100 | 90 | 100 | 75 | 90 | 90 | 95 | 95 | 70 | 85 | 60 |
| Johnsongrass | 80 | 75 | 100 | 90 | 75 | 100 | 100 | 100 | 90 | 90 | 60 | 70 | 100 | 20 | 50 | 90 | 100 | 50 | 0 | 95 | 85 | 80 | 90 | 60 | 50 | 70 | 75 |
| Kochia | 100 | 100 | 100 | 100 | 95 | 100 | 100 | 100 | 90 | 100 | 90 | 100 | — | 30 | 10 | 100 | 10 | 80 | 100 | 100 | 95 | 100 | 95 | 90 | 100 | 100 | 90 |
| Lambsquarters | 100 | 100 | 100 | 100 | 100 | 100 | 95 | 100 | 100 | 100 | 100 | 100 | 100 | 100 | 100 | 100 | 100 | 95 | 100 | 100 | 100 | 100 | 100 | 100 | 100 | 100 | 100 |
| Morningglory | 0 | 15 | 60 | 0 | 100 | 70 | 0 | 100 | 20 | — | 70 | — | 0 | 20 | 0 | 90 | 90 | 0 | 0 | 20 | 55 | 25 | 50 | 10 | 5 | 10 | 40 |
| Nightshade | 95 | 85 | 100 | 85 | 100 | 95 | 100 | 100 | 100 | 100 | 100 | 100 | 90 | 90 | 100 | 100 | 100 | 95 | 80 | 90 | 95 | 100 | 100 | 90 | 40 | 95 | 95 |
| Nutsedge, Yellow | 40 | 55 | 50 | 50 | 65 | 70 | 80 | 20 | 20 | 0 | 20 | 0 | 0 | 0 | 0 | 0 | 0 | 10 | 40 | 0 | 0 | 0 | 50 | 5 | 10 | 40 | 0 |
| Pigweed | 100 | 100 | 100 | 100 | 100 | 100 | 100 | 100 | 100 | 100 | 100 | 100 | 100 | 100 | 90 | 100 | 100 | 100 | 95 | 95 | 100 | 100 | 100 | 100 | 95 | 95 | 100 |
| Ragweed | 100 | 100 | 100 | 100 | 100 | 100 | 100 | 0 | 60 | 100 | 0 | 60 | 0 | 10 | 70 | 90 | 100 | 70 | 80 | 0 | 95 | 100 | 100 | 95 | 80 | 85 | 10 |
| Soybean | 10 | 40 | 40 | 0 | 15 | 85 | 95 | 30 | 30 | 30 | 10 | 10 | 0 | 10 | 10 | 10 | 10 | 0 | 0 | 0 | 10 | 0 | 25 | 0 | 0 | 30 | 10 |
| Sunflower | 0 | 0 | 0 | 0 | 65 | 100 | 75 | 20 | 0 | 60 | 30 | 20 | 0 | 0 | 0 | 10 | 10 | 0 | 0 | 0 | 0 | 0 | 0 | 0 | 0 | 0 | 0 |
| Surinam Grass | 80 | 65 | 90 | 100 | 95 | 95 | 90 | 60 | 50 | 60 | 20 | 30 | 60 | 20 | 10 | 60 | 60 | 30 | 80 | 65 | 60 | 35 | 80 | 50 | 5 | 50 | 45 |
| Velvetleaf | 50 | 100 | — | 50 | 100 | 100 | 100 | 100 | 100 | 80 | 80 | 100 | 90 | 100 | 70 | 100 | 100 | 10 | 5 | 15 | 50 | 100 | 100 | 85 | 80 | 70 | 30 |
| | 250 g ai/ha | | | | | | | | | | | | | | | | | | | | | | | | | | |
| | Compounds | | | | | | | | | | | | | | | | | | | | | | | | | | |
| | 64 | 65 | 80 | 88 | 95 | 96 | 102 | 108 | 114 | 115 | 130 | 131 | 134 | 137 | 139 | 140 | 144 | 145 | 146 | 147 | 149 | 152 | 153 | 156 | 162 | 182 | 188 |
| Bermudagrass | 85 | 95 | 10 | 95 | 100 | 85 | 100 | 100 | 100 | 100 | 100 | 95 | 95 | 100 | 95 | 60 | 80 | 80 | 70 | 90 | 90 | 100 | 95 | 95 | 20 | 90 | 100 |
| Cocklebur | 0 | 0 | 10 | 0 | 10 | 20 | 20 | 30 | 30 | 50 | 70 | 25 | 0 | 0 | 0 | 0 | 0 | 0 | 0 | — | 0 | 65 | 0 | 100 | 65 | 0 | 0 |
| Corn | 0 | 10 | 20 | 5 | 20 | 30 | 10 | 50 | 10 | 20 | 5 | 0 | 0 | 5 | 15 | 0 | 40 | 5 | 10 | 0 | 10 | 50 | 0 | 40 | 75 | 0 | 0 |
| Crabgrass, Large | 70 | 80 | 10 | 95 | 100 | 100 | 100 | 100 | 100 | 100 | 95 | 90 | 95 | 100 | 100 | 75 | 85 | 80 | — | 90 | 100 | 100 | 0 | 90 | 80 | 75 | 95 |
| Cupgrass, Woolly | 0 | 10 | 10 | 80 | 30 | 20 | 20 | 50 | 30 | 80 | 40 | 15 | 20 | 5 | 55 | 0 | 5 | 0 | 0 | 10 | 20 | 100 | 0 | 80 | 0 | 0 | 70 |
| Foxtail, Giant | 10 | 20 | 50 | 85 | 100 | 100 | 80 | 100 | 40 | 100 | 65 | 45 | 0 | 60 | 95 | 65 | 100 | 50 | 60 | 30 | 60 | 100 | 0 | 85 | 60 | 60 | 70 |
| Goosegrass | 50 | 60 | 10 | 95 | 85 | 95 | 100 | 100 | 100 | 100 | 30 | 80 | 85 | 100 | 90 | 70 | 75 | 75 | 30 | 60 | 90 | 100 | 85 | 90 | 75 | 50 | 85 |
| Johnsongrass | 40 | 65 | 10 | 50 | 60 | 85 | 90 | 100 | 70 | 100 | 30 | 45 | 50 | 35 | 50 | 60 | 80 | 80 | 40 | 60 | 80 | 100 | 45 | 100 | 80 | 50 | 35 |
| Kochia | 60 | 85 | 10 | 90 | 100 | 100 | 40 | 100 | 100 | 100 | 20 | 100 | 100 | 100 | 100 | 95 | 100 | 60 | 85 | 60 | 60 | 100 | 100 | 100 | 100 | 65 | 0 |
| Lambsquarters | 85 | 100 | 60 | 100 | — | — | 100 | 100 | 100 | 100 | 80 | 100 | 100 | 100 | 100 | 95 | 100 | 90 | 90 | 100 | 95 | 100 | 100 | 100 | 100 | 95 | 100 |
| Morningglory | 0 | 0 | 50 | 0 | 70 | 80 | 80 | 50 | — | 80 | — | 0 | 0 | 0 | 60 | 0 | — | 0 | 40 | 0 | 5 | 100 | 15 | 100 | 100 | 65 | 60 |
| Nightshade | 95 | 100 | 10 | 95 | 95 | 95 | 100 | 100 | 90 | 100 | — | 100 | 95 | 90 | 100 | 65 | 100 | 80 | 100 | 90 | 80 | 100 | 100 | 100 | 100 | 85 | 100 |
| Nutsedge, Yellow | 0 | 0 | 0 | 0 | 0 | 0 | 30 | 80 | 0 | 0 | 0 | 0 | 0 | 0 | 5 | 0 | 10 | 0 | 0 | 0 | 0 | 40 | 0 | 85 | 55 | 0 | 0 |
| Pigweed | 100 | 100 | 100 | 100 | 100 | 100 | 100 | 100 | 100 | 100 | 100 | 100 | 100 | 100 | 100 | 100 | 100 | 90 | 95 | 90 | 100 | 100 | 100 | 100 | 100 | 95 | 95 |
| Ragweed | 0 | 0 | 10 | 5 | 100 | 100 | 90 | 20 | 30 | 100 | 60 | 90 | 90 | 70 | 95 | 70 | 50 | 0 | 50 | 0 | 5 | 100 | 15 | 100 | 100 | 60 | 75 |
| Soybean | 10 | 20 | 0 | 0 | 10 | 45 | 0 | 0 | — | 0 | 5 | 15 | 0 | 0 | 30 | 0 | 60 | 30 | 0 | 0 | 0 | 50 | 0 | 85 | 90 | 0 | 0 |
| Sunflower | 0 | 0 | 10 | 0 | 0 | 20 | 20 | 20 | 30 | 10 | 0 | 0 | 0 | 0 | 20 | 0 | 0 | 0 | 0 | 0 | 0 | 60 | 0 | 80 | 70 | 0 | 0 |
| Surinam Grass | 20 | 0 | 10 | 50 | 60 | 40 | 40 | 90 | 40 | 70 | 20 | 15 | 50 | 30 | 30 | 65 | 60 | 65 | 40 | 10 | 40 | 100 | 75 | 85 | 80 | 25 | 75 |
| Velvetleaf | 50 | 5 | 100 | 10 | 80 | 100 | 20 | 100 | 30 | 100 | 100 | 20 | 100 | 70 | 100 | 0 | 100 | 100 | 100 | 70 | 0 | 100 | 75 | 100 | 100 | 20 | 45 |
| | 250 g ai/ha | | | | | | | | | | | | | | | | | | | | | | | | | | |
| | Compounds | | | | | | | | | | | | | | | | | | | | | | | | | | |
| | 193 | 195 | 198 | 199 | 202 | 205 | 207 | 210 | 212 | 213 | 215 | 221 | 222 | 223 | 242 | 244 | 268 | 269 | 287 | 293 | 294 | 298 | 299 | 300 | 301 | 304 | 305 |
| Bermudagrass | 85 | 30 | 100 | 100 | 95 | 100 | 100 | 100 | 80 | 80 | 85 | 85 | 100 | 100 | 100 | 100 | 100 | 100 | 95 | 100 | 90 | 80 | 100 | 100 | 90 | 100 | 100 |
| Cocklebur | — | 0 | 0 | 0 | 10 | 0 | 0 | 0 | 5 | 100 | 0 | 0 | 70 | 0 | 35 | 0 | 70 | 5 | 10 | 20 | 80 | 0 | 80 | 5 | 0 | 0 | 65 |
| Corn | 95 | 0 | 0 | 0 | 0 | 0 | 5 | 20 | 30 | 30 | 20 | 0 | 60 | 5 | 20 | 10 | 60 | 40 | 50 | 80 | 60 | 15 | 60 | 35 | 40 | 15 | 45 |
| Crabgrass, Large | 85 | 5 | 95 | 90 | 95 | 100 | 100 | 95 | 85 | 100 | 85 | 100 | 100 | 100 | 100 | 100 | 100 | 100 | 95 | 100 | 100 | 100 | 100 | 100 | 100 | 100 | 100 |
| Cupgrass, Woolly | 60 | 10 | 10 | 40 | 20 | 10 | 5 | 5 | 20 | 0 | 40 | 0 | 100 | — | 95 | 85 | 95 | 60 | 10 | 95 | 80 | 30 | 90 | 60 | 70 | 60 | 60 |
| Foxtail, Giant | 80 | 0 | 60 | 80 | 80 | 80 | 10 | 10 | 20 | 95 | 70 | 40 | 95 | 100 | 100 | 95 | 95 | 100 | 85 | 100 | 90 | 70 | 100 | 100 | 95 | 60 | 100 |
| Goosegrass | 80 | 100 | 95 | 90 | 90 | 80 | 80 | 95 | 95 | 80 | 80 | 0 | 100 | 80 | 100 | 100 | 100 | 95 | 100 | 95 | 85 | 100 | 100 | 100 | 100 | 90 | 95 |
| Johnsongrass | 90 | 0 | 60 | 60 | 70 | 60 | 40 | 50 | 50 | 60 | 50 | 10 | 100 | — | 100 | 95 | 100 | 100 | 95 | 100 | 100 | 60 | 100 | 95 | 85 | 70 | 95 |
| Kochia | 100 | 10 | 100 | 90 | 85 | 90 | 90 | 90 | 90 | 100 | 100 | 65 | 100 | 100 | — | — | 100 | 100 | 100 | 100 | 100 | 100 | 100 | 100 | 100 | 85 | 100 |
| Lambsquarters | 100 | 10 | 100 | 100 | 100 | 90 | 95 | 100 | 100 | 100 | 100 | 90 | 100 | 100 | — | — | 100 | 100 | 100 | 100 | 100 | 100 | 100 | 100 | 100 | 100 | 100 |
| Morningglory | 100 | 0 | 85 | 70 | 80 | 10 | 0 | 5 | 5 | 0 | 0 | 0 | 30 | 5 | 0 | 20 | 5 | 10 | — | 100 | 50 | 20 | 100 | 40 | 10 | 10 | 5 |
| Nightshade | 85 | 0 | 100 | 100 | 100 | 100 | 100 | 100 | 100 | 100 | 100 | 70 | 100 | 100 | 100 | 100 | 100 | 100 | 100 | 100 | 100 | 100 | 100 | 100 | 100 | 100 | 100 |
| Nutsedge, Yellow | 10 | 0 | 0 | 0 | 0 | 0 | 20 | 5 | 0 | 0 | 0 | 0 | 20 | 0 | 15 | 0 | 60 | 40 | 60 | 0 | 80 | 20 | 60 | 60 | 10 | 0 | 25 |
| Pigweed | 100 | 90 | 100 | 100 | 100 | 95 | 100 | 100 | 100 | 100 | 100 | 100 | 100 | 100 | 100 | 100 | 100 | 100 | 100 | 95 | 100 | 100 | 100 | 100 | 100 | 100 | 100 |
| Ragweed | 100 | 80 | 95 | 80 | 95 | 50 | 60 | 90 | 30 | 0 | 100 | — | 100 | 100 | 100 | 100 | 100 | 90 | 100 | 100 | 100 | 0 | 100 | 10 | 65 | 70 | 85 |
| Soybean | 50 | 0 | 0 | 5 | 0 | 20 | 0 | 0 | 0 | 0 | 0 | 0 | 100 | 80 | 65 | 10 | 90 | 50 | 85 | 90 | 100 | 5 | 90 | 70 | 20 | 20 | 90 |
| Sunflower | 0 | 0 | 10 | 0 | 10 | 0 | 0 | 0 | 0 | 0 | 0 | 0 | 30 | 0 | 50 | 15 | 60 | 5 | 100 | 65 | 100 | 0 | 100 | 5 | 0 | 0 | 0 |
| Surinam Grass | 95 | 85 | 30 | 30 | 40 | 50 | 0 | 10 | 5 | 20 | 30 | 10 | 100 | 70 | 95 | 100 | 95 | 85 | 65 | 100 | 100 | 40 | 100 | 100 | 80 | 40 | 60 |
| Velvetleaf | 70 | 55 | 80 | 85 | 100 | 60 | 40 | 60 | 100 | 0 | 40 | 60 | 100 | 100 | 100 | 100 | 100 | 75 | 100 | 100 | 100 | 100 | 100 | 5 | 100 | — | 100 |
| | 250 g ai/ha | | | | | | | | | | | | | | | | | | | | | | | | | | |
| | Compounds | | | | | | | | | | | | | | | | | | | | | | | | | | |
| | 306 | 307 | 309 | 310 | 314 | 318 | 332 | 333 | 334 | 336 | 340 | 341 | 342 | 343 | 352 | 372 | 375 | 376 | 382 | 384 | 385 | 386 | 391 | 392 | 393 | 395 | 396 |
| Bermudagrass | 90 | 100 | 95 | 100 | 60 | 90 | 100 | 100 | 100 | 100 | 85 | 100 | 90 | 90 | 100 | 98 | 100 | 100 | 85 | 100 | 100 | 95 | 95 | 95 | 100 | 100 | 100 |
| Cocklebur | 0 | 35 | 60 | 5 | 0 | 0 | 0 | 0 | 70 | 30 | 0 | 50 | 60 | 80 | 0 | 60 | 0 | 0 | 20 | — | 100 | 30 | 80 | 20 | 100 | 80 | 0 |
| Corn | 20 | 30 | 40 | 5 | 0 | 10 | 0 | 0 | 50 | 85 | 45 | 85 | 70 | 70 | 30 | 65 | 5 | 45 | 55 | — | — | 80 | 85 | 80 | 75 | 85 | 60 |
| Crabgrass, Large | 100 | 100 | 100 | 100 | 40 | 60 | 100 | 100 | 100 | 100 | 100 | 90 | 70 | 80 | 100 | 95 | 100 | 100 | 100 | 100 | 100 | 100 | 100 | 100 | 100 | 100 | 100 |
| Cupgrass, Woolly | 30 | 70 | 90 | 70 | 0 | 0 | 5 | 0 | 85 | 60 | 45 | 100 | 50 | 70 | 60 | 70 | 70 | 75 | 20 | 95 | 80 | 95 | 80 | 60 | 90 | 100 | 100 |
| Foxtail, Giant | 90 | 100 | 90 | 85 | 20 | 50 | 60 | 50 | 100 | 90 | 60 | 55 | 55 | 70 | 100 | 90 | — | 95 | 100 | 100 | 95 | 90 | 100 | 100 | 95 | 100 | 100 |
| Goosegrass | 100 | 100 | 100 | 95 | 65 | 80 | 100 | 85 | 100 | 100 | 85 | 85 | 65 | 80 | 100 | 90 | 100 | 100 | 95 | 100 | 95 | 95 | 90 | 95 | 100 | 100 | 100 |
| Johnsongrass | 80 | 90 | 95 | 20 | 70 | 60 | 50 | 5 | 100 | 100 | 50 | 100 | 100 | 100 | 90 | 90 | 5 | 100 | 100 | 100 | 100 | 100 | 100 | 100 | 100 | 100 | 100 |
| Kochia | 100 | 100 | 100 | 100 | 30 | 85 | 100 | 100 | 100 | 100 | 90 | 100 | 100 | 100 | 100 | 100 | 100 | 100 | 95 | 100 | 85 | 100 | 100 | 100 | 100 | 100 | 100 |
| Lambsquarters | 100 | 100 | 100 | 100 | 30 | 100 | 100 | 100 | 100 | 100 | 100 | 100 | 100 | 100 | 100 | 100 | 90 | 100 | 100 | 100 | 100 | 100 | 100 | 100 | 100 | 100 | 100 |
| Morningglory | 10 | 10 | 5 | 5 | 30 | 0 | 0 | 0 | 40 | 100 | 10 | 5 | 70 | 100 | 40 | 40 | 5 | 5 | 65 | — | 65 | 100 | 100 | 100 | — | 100 | 25 |
| Nightshade | 100 | 100 | 100 | 100 | 90 | 95 | 95 | 98 | 100 | 100 | 100 | 100 | 80 | 90 | 50 | 100 | 55 | 100 | 95 | — | — | 95 | 95 | 100 | — | 100 | 100 |
| Nutsedge, Yellow | 10 | 50 | 45 | 0 | 0 | 0 | 0 | 0 | 55 | 20 | 0 | 30 | 40 | 60 | 10 | 40 | 0 | 40 | 25 | — | 100 | 40 | 90 | 30 | 50 | 80 | 45 |
| Pigweed | 100 | 100 | 100 | 100 | 90 | 100 | 100 | 100 | 100 | 100 | 100 | 100 | 100 | 100 | 100 | 100 | 100 | 100 | 100 | 100 | 100 | 100 | 100 | 100 | 100 | 100 | 100 |
| Ragweed | — | 100 | 70 | 50 | 40 | 70 | 5 | 50 | 100 | 80 | 20 | 100 | 100 | 90 | 50 | 100 | 10 | 50 | 90 | 100 | 90 | 100 | 100 | 100 | 100 | 100 | 80 |
| Soybean | 15 | 80 | 80 | 30 | 40 | 10 | 0 | 0 | 80 | 50 | 0 | 70 | 100 | 100 | 50 | 20 | 0 | 10 | 100 | — | 100 | 95 | 100 | 90 | — | 100 | 100 |
| Sunflower | 0 | 60 | 5 | 0 | 20 | 0 | 0 | 0 | 55 | 30 | 0 | 50 | 75 | 80 | 0 | 0 | 0 | 0 | 0 | — | — | 85 | 100 | — | — | 50 | 0 |
| Surinam Grass | 40 | 90 | 90 | 30 | 0 | 50 | 10 | 5 | 95 | 100 | 65 | 100 | 100 | 100 | 100 | 85 | 80 | 90 | 100 | — | — | 95 | 95 | 85 | — | 100 | 100 |
| Velvetleaf | — | 100 | 75 | 10 | 80 | 100 | 45 | 50 | 85 | 100 | 95 | 100 | 100 | 100 | 70 | 100 | 80 | — | 100 | 100 | 100 | 100 | 100 | 100 | 100 | 100 | 100 |
| | 250 g ai/ha | | | | | | | 125 g ai/ha | | | | | | | | | | | | | | | | | | | |
| | Compounds | | | | | | | Compounds | | | | | | | | | | | | | | | | | | | |
| | 399 | 409 | 414 | 417 | 418 | 419 | 420 | 1 | 2 | 3 | 4 | 5 | 6 | 7 | 9 | 10 | 11 | 13 | 14 | 16 | 27 | 28 | 29 | 30 | 31 | 33 | 34 |
| Bermudagrass | 100 | 85 | 100 | 100 | 100 | 100 | 100 | 85 | 90 | 90 | 95 | 10 | 90 | 70 | 65 | 50 | 30 | 80 | 55 | 75 | 100 | 90 | 100 | 90 | 90 | 90 | 50 |
| Cocklebur | 70 | 70 | 20 | 0 | 30 | 45 | 15 | — | 5 | 5 | 60 | 0 | 100 | 0 | 0 | 0 | 10 | 0 | 65 | 0 | 10 | 0 | — | 0 | 20 | 30 | 0 |
| Corn | 80 | 75 | 50 | 10 | 40 | 0 | 10 | 45 | 30 | 0 | 0 | 0 | 60 | 30 | 25 | 40 | 70 | 50 | 70 | 55 | 0 | 0 | 0 | 0 | 20 | 0 | 0 |
| Crabgrass, Large | 100 | 95 | 100 | 100 | 100 | 100 | 100 | 80 | 95 | 80 | 80 | 70 | 95 | 85 | 80 | 90 | 80 | 100 | 100 | 95 | 100 | 100 | 100 | 90 | 100 | 100 | 100 |
| Cupgrass, Woolly | 80 | 80 | 95 | 65 | 10 | 45 | 20 | 35 | 60 | 30 | 20 | 0 | 55 | 30 | 15 | 0 | 55 | 20 | 60 | 45 | 20 | 20 | 20 | 10 | 0 | 50 | 20 |
| Foxtail, Giant | 100 | 95 | 100 | 95 | 100 | 95 | 100 | 65 | 100 | 90 | 70 | 5 | 80 | 70 | 60 | 70 | 60 | 20 | 95 | 70 | 100 | 20 | 80 | 40 | 30 | 60 | 70 |
| Goosegrass | 100 | 95 | 100 | 100 | 100 | 100 | 100 | 85 | 95 | 90 | 85 | 50 | 100 | 70 | 80 | 80 | 70 | 90 | 95 | 80 | 90 | 70 | 60 | 30 | 30 | 90 | 80 |
| Johnsongrass | 100 | 95 | 100 | 100 | 95 | 100 | 100 | 75 | 90 | 70 | 65 | 20 | 90 | 50 | 55 | 90 | 65 | 60 | 85 | 80 | 90 | 60 | — | — | 30 | 80 | 80 |
| Kochia | 100 | 85 | 80 | 100 | 85 | 100 | 100 | 100 | 100 | 80 | — | 10 | 100 | 80 | 100 | 100 | 100 | 90 | 100 | 100 | 100 | 0 | 80 | 70 | 80 | 100 | 90 |
| Lambsquarters | 100 | 100 | 95 | 100 | 100 | 100 | 100 | 100 | 100 | 90 | 100 | 60 | 100 | 85 | 100 | 100 | 100 | 100 | 100 | 95 | 100 | 100 | 90 | 100 | 100 | 100 | 100 |
| Morningglory | 45 | 100 | 90 | 80 | 95 | 95 | 95 | 0 | 20 | 20 | 20 | 40 | 0 | 0 | 0 | 10 | 0 | 90 | 65 | 0 | 100 | 20 | 100 | — | 40 | 0 | 0 |
| Nightshade | 100 | 95 | 100 | 95 | 95 | 100 | 95 | 100 | 100 | 95 | 95 | 100 | 100 | 60 | 75 | 100 | 80 | 90 | 90 | 100 | 100 | 100 | 100 | 90 | 90 | 100 | 90 |
| Nutsedge, Yellow | 85 | 30 | 0 | 0 | 0 | 0 | 0 | 25 | 65 | 30 | 25 | 0 | 70 | 20 | 25 | 30 | 0 | 25 | 55 | 75 | 20 | 0 | — | 0 | 0 | 0 | 0 |
| Pigweed | 100 | 100 | 100 | 100 | 100 | 100 | 100 | 100 | 95 | 95 | 95 | 95 | 100 | 100 | 95 | 100 | 100 | 100 | 100 | 100 | 100 | 100 | 100 | 100 | 100 | 100 | 100 |
| Ragweed | 100 | 90 | 55 | 80 | 75 | 100 | 80 | 100 | 100 | 95 | 80 | 95 | 100 | 50 | 100 | 100 | 100 | 100 | 100 | 100 | 0 | 20 | 0 | 0 | 0 | 10 | 0 |
| Soybean | 90 | 95 | 55 | 0 | 75 | 95 | 15 | 0 | 40 | 10 | 35 | 0 | 90 | 0 | 20 | 0 | 0 | 0 | 35 | 75 | 10 | 0 | 30 | 0 | 0 | 0 | 0 |
| Sunflower | 25 | 25 | 0 | 0 | 0 | 85 | 0 | 0 | 0 | 0 | 0 | 0 | 50 | 0 | 0 | 0 | 0 | 0 | 75 | 20 | 20 | 0 | 60 | 10 | 20 | 0 | 0 |
| Surinam Grass | 100 | 95 | 95 | 80 | 100 | 100 | 95 | 75 | 95 | 70 | 50 | 55 | 100 | 50 | 45 | 40 | 95 | 85 | 95 | 75 | 30 | 20 | 30 | 20 | 20 | 30 | 20 |
| Velvetleaf | 100 | 100 | 100 | 100 | 100 | 100 | 100 | 85 | 100 | 85 | 90 | 30 | 100 | 0 | 50 | — | 10 | 100 | 100 | 100 | 100 | 70 | 80 | 30 | 70 | 70 | 60 |
| | 125 g ai/ha | | | | | | | | | | | | | | | | | | | | | | | | | | |
| | Compounds | | | | | | | | | | | | | | | | | | | | | | | | | | |
| | 35 | 41 | 44 | 46 | 47 | 48 | 50 | 55 | 56 | 57 | 58 | 60 | 61 | 62 | 63 | 64 | 65 | 80 | 88 | 95 | 96 | 102 | 108 | 114 | 115 | 118 | 128 |
| Bermudagrass | 100 | 50 | — | 100 | 100 | 90 | 0 | 100 | 90 | 95 | 100 | 100 | 70 | 90 | 90 | 30 | 85 | 10 | 95 | 100 | 80 | 100 | 100 | 90 | 100 | 100 | 100 |
| Cocklebur | 20 | 0 | — | 40 | 0 | 0 | 10 | 0 | 0 | 0 | 5 | 0 | 0 | 0 | 0 | 0 | 0 | 10 | 0 | 5 | 5 | 20 | 30 | 20 | 40 | 20 | 10 |
| Corn | 0 | 0 | 0 | 0 | 0 | 0 | 0 | 0 | 0 | 0 | 0 | 0 | 0 | 0 | 0 | 0 | 0 | 10 | 5 | 10 | 15 | 0 | 40 | 10 | 10 | 10 | 20 |
| Crabgrass, Large | 50 | 100 | 20 | 100 | 100 | 30 | 0 | 90 | 100 | 100 | 95 | 90 | 75 | 90 | 85 | 40 | 50 | 10 | 95 | 100 | 100 | 100 | 100 | 100 | 100 | 100 | 100 |
| Cupgrass, Woolly | 20 | 0 | 10 | 10 | 10 | 0 | 10 | 25 | 0 | 0 | 40 | 10 | 0 | 0 | 0 | 0 | 0 | 10 | 30 | 10 | 10 | 10 | 50 | 10 | 30 | 0 | 20 |
| Foxtail, Giant | 30 | 20 | 10 | 60 | 70 | 0 | 0 | 50 | 85 | 95 | 100 | 15 | 20 | 65 | 5 | 0 | 0 | 40 | 50 | 100 | 100 | 50 | 100 | 30 | 90 | 10 | 10 |
| Goosegrass | 70 | 30 | 30 | 90 | 100 | 70 | 80 | 50 | 85 | 75 | 95 | 85 | 40 | 70 | 45 | 10 | 30 | 10 | 90 | 50 | 80 | 100 | 100 | 80 | 80 | 80 | 90 |
| Johnsongrass | 70 | — | 30 | 80 | 100 | 20 | — | 75 | 70 | 65 | 80 | 50 | 30 | 40 | 60 | 20 | 40 | 10 | 20 | 40 | 60 | 80 | 100 | 30 | 100 | 100 | 90 |
| Kochia | 10 | 20 | 10 | 90 | 10 | 60 | 85 | 80 | 90 | 100 | 95 | 90 | 100 | 100 | 80 | 20 | 70 | 10 | 85 | 100 | 95 | 30 | 100 | 90 | 100 | 20 | 0 |
| Lambsquarters | 100 | 90 | 100 | 100 | 100 | 95 | 85 | 95 | 100 | 100 | 100 | 100 | 100 | 100 | 95 | 0 | 95 | 10 | 95 | — | — | 70 | 100 | 100 | 100 | 60 | 30 |
| Morningglory | — | 0 | 0 | 0 | 0 | — | 0 | 0 | 0 | 0 | 20 | 5 | 0 | 0 | 40 | 0 | 0 | — | 0 | 10 | 50 | 70 | — | 60 | 30 | 30 | 30 |
| Nightshade | 90 | 10 | 90 | 100 | 100 | 80 | 5 | 60 | 95 | 95 | 100 | 90 | 20 | 90 | 90 | 50 | 90 | 10 | 90 | 95 | 95 | 100 | 100 | 90 | 100 | 100 | 90 |
| Nutsedge, Yellow | 0 | 0 | 0 | 0 | 0 | 0 | 10 | 0 | 0 | 0 | 20 | 0 | 0 | 10 | 0 | 0 | 0 | 0 | 0 | 0 | 0 | 0 | 70 | 0 | 0 | 0 | 70 |
| Pigweed | 100 | 90 | 70 | 100 | 100 | 100 | 90 | 95 | 100 | 100 | 100 | 100 | 95 | 95 | 100 | 70 | 100 | 100 | 100 | 100 | 100 | 100 | 100 | 100 | 100 | 90 | 60 |
| Ragweed | 80 | 0 | 30 | 40 | 80 | 5 | 40 | 0 | 95 | 100 | 85 | 80 | 0 | 20 | 0 | 0 | 0 | 0 | 0 | 80 | 90 | 20 | 20 | 10 | 80 | 10 | 20 |
| Soybean | 0 | 0 | 10 | 0 | 0 | 0 | 0 | 0 | 0 | 0 | 5 | 0 | 0 | 0 | 0 | 0 | 0 | 0 | 0 | 0 | 5 | 0 | 0 | — | 0 | 10 | 20 |
| Sunflower | 10 | 0 | 0 | 10 | 0 | 0 | 0 | 0 | 0 | 0 | 0 | 0 | 0 | 0 | 0 | 0 | 0 | 0 | 0 | 0 | 0 | 20 | 20 | 30 | — | 20 | 50 |
| Surinam Grass | 20 | 20 | 10 | 30 | 10 | 5 | 10 | 25 | 15 | 15 | 55 | 0 | 0 | 10 | 5 | 5 | 0 | 10 | 20 | 10 | 10 | — | 40 | 10 | 40 | 20 | 60 |
| Velvetleaf | 100 | 40 | 70 | 100 | 40 | 0 | 5 | 0 | 35 | 0 | 100 | 70 | 45 | — | 10 | 30 | 0 | 100 | 0 | 60 | 50 | 20 | 90 | 30 | 100 | 40 | 100 |
| | 125 g ai/ha | | | | | | | | | | | | | | | | | | | | | | | | | | |
| | Compounds | | | | | | | | | | | | | | | | | | | | | | | | | | |
| | 130 | 131 | 134 | 137 | 139 | 140 | 144 | 145 | 146 | 147 | 149 | 152 | 153 | 156 | 162 | 182 | 188 | 193 | 195 | 198 | 199 | 202 | 205 | 207 | 210 | 212 | 213 |
| Bermudagrass | 90 | 90 | 95 | 100 | 90 | 0 | 80 | 60 | 60 | 70 | 90 | 90 | 95 | 93 | 0 | 75 | 100 | 80 | 0 | 85 | 85 | 85 | 100 | 80 | 80 | 75 | 60 |
| Cocklebur | — | 20 | 0 | 0 | 0 | 0 | 0 | 0 | 0 | — | 0 | 65 | 0 | 98 | — | 0 | 0 | — | 0 | 0 | 0 | 0 | 0 | 0 | 0 | 0 | — |
| Corn | 5 | 0 | 0 | 5 | 0 | 0 | 30 | 0 | 0 | 0 | 0 | 45 | 0 | 38 | 70 | 0 | 0 | 60 | 0 | 0 | 0 | 0 | 0 | 0 | 10 | 0 | 0 |
| Crabgrass, Large | 90 | 90 | 95 | 70 | 100 | 65 | 70 | 50 | — | 50 | 90 | 100 | 0 | 85 | 40 | 25 | 95 | 85 | 0 | 85 | 90 | 95 | 80 | 90 | 80 | 80 | 50 |
| Cupgrass, Woolly | 10 | 0 | 0 | 0 | 10 | 0 | 5 | 0 | 0 | 10 | 5 | 50 | 0 | 70 | 0 | 0 | 60 | 40 | 0 | 5 | 0 | 10 | 10 | 0 | 0 | 0 | 0 |
| Foxtail, Giant | 45 | 20 | 0 | 50 | 80 | 25 | 70 | 0 | 10 | 0 | 50 | 100 | 0 | 83 | 60 | 0 | 55 | 50 | 0 | 10 | 10 | 40 | 10 | 0 | 10 | 20 | 50 |
| Goosegrass | 20 | 65 | 65 | 85 | 85 | 0 | 60 | 70 | 0 | 20 | 70 | 100 | 75 | 90 | 60 | 25 | 70 | 80 | 100 | 80 | 70 | 85 | 70 | 60 | 80 | 60 | 60 |
| Johnsongrass | — | 40 | 40 | 5 | 30 | 45 | 0 | 65 | 30 | 40 | 60 | 100 | 15 | 85 | 80 | 35 | 20 | 85 | 0 | 30 | 30 | 40 | 10 | 10 | 20 | 0 | 55 |
| Kochia | 0 | 85 | 100 | 90 | 100 | 75 | 80 | 60 | 50 | 40 | 10 | 100 | 95 | 95 | 75 | 60 | 0 | 85 | 0 | 80 | 85 | 85 | 90 | 90 | 90 | 85 | 85 |
| Lambsquarters | 30 | 100 | 100 | 100 | 100 | 85 | 95 | 60 | 90 | 90 | 95 | 100 | 100 | 100 | 95 | 95 | 100 | 100 | 0 | 100 | 95 | 90 | 90 | 95 | 100 | 100 | 95 |
| Morningglory | 0 | 0 | 0 | 0 | 10 | 0 | 30 | 0 | 5 | 0 | 5 | 80 | 0 | 60 | 100 | 15 | — | 20 | 0 | 70 | 50 | 55 | 0 | 0 | 0 | 0 | 0 |
| Nightshade | — | 95 | 95 | 85 | 100 | 0 | 100 | 70 | 60 | 70 | 80 | 90 | 100 | 93 | 100 | 80 | 95 | 50 | 0 | 95 | 95 | 95 | 100 | 75 | 100 | 100 | 100 |
| Nutsedge, Yellow | 0 | 0 | 0 | 0 | 0 | 0 | 0 | 0 | 0 | 0 | 0 | 30 | 0 | 60 | 20 | 0 | 0 | 0 | 0 | 0 | 0 | 0 | 0 | 0 | 0 | 0 | 0 |
| Pigweed | 90 | 100 | 95 | 100 | 100 | 100 | 90 | 90 | 95 | 90 | 100 | 100 | 100 | 98 | 100 | 95 | 95 | 100 | 60 | 100 | 100 | 100 | 95 | 100 | 100 | 100 | 100 |
| Ragweed | 10 | 50 | 25 | 0 | 50 | 0 | 30 | 0 | 0 | 0 | 0 | 85 | 0 | 100 | 95 | 20 | 65 | 100 | 40 | 80 | 60 | 80 | 20 | 50 | 0 | 0 | 0 |
| Soybean | 0 | 0 | 0 | 0 | 10 | — | 40 | 10 | 0 | 0 | 0 | 10 | 0 | 50 | 75 | 0 | 0 | 30 | 0 | 0 | 0 | 0 | 20 | 0 | 0 | 0 | 0 |
| Sunflower | 0 | 0 | 0 | 0 | 10 | 0 | 0 | 0 | 0 | 0 | 0 | 10 | 0 | 45 | 15 | 0 | 0 | 0 | 0 | 0 | 0 | 0 | 0 | 0 | 0 | 0 | 0 |
| Surinam Grass | 5 | 0 | 15 | 10 | 20 | 0 | 40 | 50 | 0 | 10 | 5 | 60 | 0 | 88 | 75 | 20 | 30 | 80 | 0 | 10 | 0 | 20 | 20 | 0 | 5 | 0 | 0 |
| Velvetleaf | 10 | 15 | 90 | 10 | 30 | 0 | 100 | 20 | 40 | 20 | 0 | 100 | 20 | 63 | 75 | 0 | 0 | 0 | 50 | 80 | 70 | 10 | 40 | 10 | 0 | 100 | 0 |
| | 125 g ai/ha | | | | | | | | | | | | | | | | | | | | | | | | | | |
| | Compounds | | | | | | | | | | | | | | | | | | | | | | | | | | |
| | 215 | 221 | 222 | 223 | 242 | 244 | 268 | 269 | 287 | 293 | 294 | 298 | 299 | 300 | 301 | 304 | 305 | 306 | 307 | 309 | 310 | 314 | 318 | 332 | 333 | 334 | 336 |
| Bermudagrass | 70 | 10 | 100 | 80 | 100 | 100 | 100 | 100 | 95 | 95 | 80 | 55 | 100 | 100 | 90 | 70 | 85 | 85 | 100 | 90 | 85 | 60 | 70 | 100 | 85 | 100 | 100 |
| Cocklebur | 0 | 0 | 20 | 0 | 0 | 0 | 60 | 0 | 0 | — | 40 | 0 | 40 | 5 | 0 | 0 | 15 | 0 | 30 | 10 | 0 | 0 | 0 | 0 | 0 | 60 | 5 |
| Corn | 20 | 0 | 20 | 0 | 0 | 10 | 45 | 20 | — | 10 | 60 | 10 | 25 | 30 | 10 | 0 | 40 | 10 | 20 | 20 | 0 | 0 | 0 | 0 | 0 | 20 | 35 |
| Crabgrass, Large | 70 | 100 | 100 | 100 | 100 | 100 | 95 | 100 | 95 | 95 | 95 | 95 | 100 | 100 | 100 | 100 | 100 | 100 | 100 | 80 | 70 | 0 | 50 | 100 | 70 | 100 | 100 |
| Cupgrass, Woolly | 10 | 0 | 100 | 10 | 80 | 65 | 70 | 40 | 0 | 70 | 75 | 10 | 70 | 50 | 50 | 10 | 60 | 15 | 40 | 70 | 10 | 0 | 0 | 0 | 0 | 60 | 10 |
| Foxtail, Giant | 50 | 0 | 85 | 70 | 100 | 95 | 95 | 90 | 70 | 70 | 80 | 70 | 100 | 65 | 60 | 50 | 85 | 40 | 85 | 80 | 40 | 0 | 10 | 20 | 10 | 80 | 5 |
| Goosegrass | 40 | 0 | 100 | 80 | 100 | 95 | 90 | 85 | 100 | 85 | 80 | 95 | 100 | 100 | 90 | 85 | 85 | 70 | 95 | 85 | 80 | 40 | 10 | 85 | 30 | 100 | 100 |
| Johnsongrass | 20 | 0 | 100 | 80 | 95 | 95 | 90 | 60 | 85 | 95 | 100 | 40 | 100 | 85 | — | 60 | 90 | 60 | 80 | 80 | 10 | 20 | 0 | 10 | 0 | 100 | 95 |
| Kochia | 100 | 0 | 100 | 90 | — | — | 100 | 100 | 100 | 100 | 100 | 95 | 100 | 100 | 100 | 80 | 100 | 80 | 100 | 100 | 100 | 0 | 60 | 100 | 100 | 100 | 70 |
| Lambsquarters | 100 | 20 | 100 | 100 | — | — | 100 | 100 | 100 | 100 | 100 | 100 | 100 | 100 | 100 | 100 | 100 | 100 | 100 | 100 | 100 | 10 | 95 | 100 | 100 | 100 | 100 |
| Morningglory | 0 | 0 | 20 | 0 | 0 | 15 | 0 | 5 | — | — | 30 | 5 | 75 | 40 | 5 | 10 | 0 | 5 | 10 | 5 | 5 | 10 | 0 | 0 | 0 | 20 | 70 |
| Nightshade | 100 | 50 | 100 | 100 | 100 | 100 | 100 | 100 | 100 | 100 | 100 | 100 | 100 | 100 | 95 | 100 | 90 | 95 | 100 | 80 | 35 | 85 | 90 | 90 | 20 | 100 | 95 |
| Nutsedge, Yellow | 0 | 0 | 0 | 0 | 0 | 0 | 50 | 10 | 15 | 0 | 55 | 0 | 50 | 20 | 5 | 0 | 20 | 0 | 20 | 30 | 0 | 0 | 0 | 0 | 0 | 40 | 5 |
| Pigweed | 100 | 100 | 100 | 100 | 100 | 100 | 100 | 100 | 100 | 85 | 100 | 100 | 100 | 100 | 100 | 100 | 100 | 100 | 100 | 100 | 100 | 60 | 95 | 100 | 100 | 100 | 100 |
| Ragweed | 0 | 0 | 100 | 80 | 100 | 100 | 90 | 10 | 100 | 100 | 100 | 0 | 100 | 5 | 60 | 0 | 85 | 70 | 100 | — | 0 | 0 | 40 | 5 | 0 | 100 | 55 |
| Soybean | 0 | 0 | 70 | 30 | 65 | 0 | 75 | 10 | 60 | 85 | 95 | 0 | 80 | 30 | 20 | 0 | 60 | 10 | 60 | 40 | 0 | 10 | 0 | 0 | 0 | 80 | 25 |
| Sunflower | 0 | 0 | 0 | 0 | 0 | 0 | 5 | 0 | 100 | — | 50 | 0 | 85 | 0 | 0 | 0 | 0 | 0 | 10 | 0 | 0 | 0 | 0 | 0 | 0 | 35 | 10 |
| Surinam Grass | 0 | 0 | 100 | 65 | 95 | 100 | 70 | 70 | 50 | 80 | 95 | 5 | 90 | 65 | 30 | 5 | 55 | 10 | 85 | 70 | 10 | 0 | 20 | 5 | 0 | 70 | 85 |
| Velvetleaf | 0 | 0 | 50 | 0 | 100 | 100 | 100 | 70 | 95 | 100 | 100 | 60 | 80 | 5 | 70 | 0 | 100 | 5 | 100 | 70 | 10 | 0 | 70 | 45 | 0 | 50 | 30 |
| | 125 g ai/ha | | | | | | | | | | | | | | | | | | | | | | | | 62 g ai/ha | | |
| | Compounds | | | | | | | | | | | | | | | | | | | | | | | | Compounds | | |
| | 340 | 341 | 342 | 343 | 352 | 372 | 375 | 376 | 382 | 384 | 385 | 386 | 391 | 392 | 393 | 395 | 396 | 399 | 409 | 414 | 417 | 418 | 419 | 420 | 1 | 2 | 3 |
| Bermudagrass | 70 | 80 | 60 | 70 | 100 | 98 | 100 | 90 | 80 | 100 | 85 | 95 | 95 | 90 | 100 | 100 | 100 | 100 | 50 | 100 | 100 | 100 | 100 | 100 | 80 | 85 | 60 |
| Cocklebur | 0 | 10 | 30 | 80 | 0 | 10 | 0 | 0 | 15 | — | 0 | 20 | 45 | 5 | 100 | 15 | 0 | 65 | 30 | 15 | 0 | 0 | 25 | 0 | — | 5 | 0 |
| Corn | 45 | 60 | 50 | 70 | 15 | 60 | 5 | 40 | 50 | — | — | 60 | 80 | 50 | — | 45 | 30 | 70 | 5 | 0 | 0 | 10 | 0 | 0 | 25 | 0 | 0 |
| Crabgrass, Large | 70 | 80 | 60 | 80 | 85 | 85 | 70 | 100 | 100 | 100 | 95 | 100 | 100 | 100 | 100 | 100 | 100 | 100 | 95 | 100 | 100 | 100 | 100 | 100 | 65 | 95 | 70 |
| Cupgrass, Woolly | 20 | 80 | 40 | 65 | 50 | 60 | 70 | 30 | 0 | 75 | 60 | 90 | 75 | 40 | 70 | 95 | 90 | 65 | 55 | 80 | 20 | 0 | 15 | 10 | — | 40 | 0 |
| Foxtail, Giant | 10 | 50 | 40 | 50 | 60 | 85 | 80 | 85 | 90 | 100 | 80 | 90 | 100 | 90 | 90 | 100 | 100 | 100 | 85 | 95 | 90 | 95 | 85 | 90 | 55 | 80 | 10 |
| Goosegrass | 60 | 85 | 65 | 75 | 85 | 80 | 100 | 100 | 90 | 95 | 80 | 95 | 85 | 95 | 100 | 95 | 100 | 100 | 90 | 100 | 100 | 100 | 95 | 95 | 80 | 90 | 60 |
| Johnsongrass | 50 | 85 | 100 | 80 | 90 | 80 | 5 | 80 | 95 | 100 | 95 | 100 | 100 | 100 | 100 | 100 | 100 | 100 | 80 | 95 | 100 | 95 | 100 | 100 | 70 | 70 | 50 |
| Kochia | 20 | 100 | 100 | 100 | 85 | 100 | 100 | 100 | 20 | 100 | 70 | 100 | 100 | 80 | 100 | 100 | 45 | 80 | 70 | 75 | 85 | 80 | 95 | 85 | 95 | 100 | 50 |
| Lambsquarters | 80 | 100 | 90 | 100 | 100 | 100 | 10 | 100 | 100 | 100 | 100 | 100 | 100 | 100 | 100 | 100 | 100 | 100 | — | 95 | 100 | 100 | 95 | 100 | 100 | 100 | 90 |
| Morningglory | 5 | 5 | 5 | 60 | 5 | 5 | 0 | 0 | 25 | — | — | 95 | 100 | 95 | — | 80 | 0 | 25 | 40 | 55 | 20 | 70 | 25 | 55 | 0 | 5 | — |
| Nightshade | 90 | 55 | 80 | 60 | 0 | 95 | 50 | 100 | — | — | — | 95 | 95 | 95 | — | 100 | 100 | 100 | 85 | 90 | 60 | 95 | 95 | 90 | 100 | 100 | 90 |
| Nutsedge, Yellow | 0 | 0 | 0 | 10 | 0 | 30 | 0 | 30 | 0 | — | 100 | 10 | 70 | 10 | — | 55 | 25 | 75 | 0 | 0 | 0 | 0 | 0 | 0 | 15 | 30 | 0 |
| Pigweed | 85 | 95 | 90 | 90 | 85 | 95 | 50 | 100 | 95 | 100 | 100 | 100 | 100 | 100 | 100 | 100 | 100 | 100 | — | 100 | 100 | 100 | 100 | 100 | 100 | 95 | 90 |
| Ragweed | 0 | 100 | 95 | 90 | 40 | 90 | 5 | 0 | 65 | 100 | 20 | 98 | 100 | 100 | 100 | 95 | 0 | 100 | 85 | 45 | 20 | 70 | 15 | 55 | 100 | 80 | 85 |
| Soybean | 0 | 20 | 60 | 80 | 15 | 5 | 0 | 10 | 100 | — | 100 | 90 | 95 | 80 | — | 85 | 75 | 85 | 80 | 20 | 0 | 20 | 85 | 10 | 0 | 30 | 10 |
| Sunflower | 0 | 0 | 20 | 75 | 0 | 0 | 0 | 0 | — | — | — | 40 | 95 | 50 | — | 0 | 0 | 0 | 5 | 0 | 0 | 0 | 0 | 0 | 0 | 0 | 0 |
| Surinam Grass | 60 | 95 | 90 | 85 | 50 | 85 | 75 | 60 | 95 | — | — | 80 | 95 | 85 | — | 100 | 95 | 95 | 85 | 80 | 75 | 85 | 45 | 20 | 50 | 90 | 60 |
| Velvetleaf | 60 | 60 | 60 | 80 | 5 | 100 | 30 | 5 | 100 | 100 | 85 | 100 | 100 | 100 | 100 | 100 | 45 | 100 | 30 | 100 | 100 | 95 | 100 | 100 | 70 | 60 | 80 |
| | 62 g ai/ha | | | | | | | | | | | | | | | | | | | | | | | | | | |
| | Compounds | | | | | | | | | | | | | | | | | | | | | | | | | | |
| | 4 | 5 | 6 | 7 | 9 | 10 | 11 | 13 | 14 | 16 | 27 | 28 | 29 | 30 | 31 | 33 | 34 | 35 | 41 | 44 | 46 | 47 | 48 | 50 | 55 | 56 | 57 |
| Bermudagrass | 85 | 0 | 80 | 60 | 65 | 50 | 0 | 0 | 45 | 70 | 90 | 90 | 90 | 10 | 20 | 80 | 20 | 90 | 30 | 0 | 90 | 90 | 80 | 0 | 60 | 85 | 55 |
| Cocklebur | 50 | 0 | 60 | 0 | 0 | — | — | 0 | 0 | 0 | 0 | 0 | 30 | 0 | 0 | 0 | 0 | 0 | 0 | 0 | 0 | 0 | 0 | 0 | 0 | 0 | 0 |
| Corn | 0 | 0 | 60 | 0 | 15 | 35 | 25 | 0 | 60 | 20 | 0 | 0 | 0 | 0 | 0 | 0 | 0 | 0 | 0 | 0 | 0 | 0 | 0 | 0 | 0 | 0 | 0 |
| Crabgrass, Large | 60 | 30 | 75 | 60 | 40 | 10 | 80 | 100 | 95 | 80 | 100 | 100 | 100 | 90 | 90 | 100 | 80 | 10 | 90 | 0 | 100 | 100 | 0 | 0 | 15 | 95 | 95 |
| Cupgrass, Woolly | 0 | 0 | 50 | 0 | 0 | 0 | 10 | 0 | 20 | 15 | 10 | 20 | 0 | 10 | 0 | 20 | 10 | 10 | 0 | 0 | 10 | 10 | 0 | 0 | 0 | 0 | 0 |
| Foxtail, Giant | 20 | 0 | 60 | 0 | 45 | 40 | 5 | 0 | 65 | 25 | 50 | 0 | 20 | 20 | 20 | 20 | 10 | 20 | 20 | 10 | 10 | 40 | 0 | 0 | 0 | 45 | 20 |
| Goosegrass | 60 | 0 | 85 | 0 | 65 | 60 | 50 | 85 | 95 | 75 | 30 | 30 | 30 | 20 | 30 | 60 | 20 | 30 | 20 | 10 | 60 | 80 | 20 | 40 | 0 | 60 | 45 |
| Johnsongrass | 30 | 0 | 70 | 10 | 45 | 60 | 50 | 0 | 70 | 50 | 60 | 20 | 90 | 0 | 0 | 20 | 50 | 50 | 0 | 30 | 50 | 20 | 10 | 0 | 0 | 55 | 20 |
| Kochia | — | 0 | 100 | 30 | 95 | 80 | 85 | 50 | 100 | 95 | 90 | 0 | 20 | 0 | 0 | 100 | 90 | 10 | 20 | 0 | 0 | 10 | 10 | 30 | 25 | 75 | 100 |
| Lambsquarters | 90 | 40 | 100 | 20 | 95 | 95 | 95 | 95 | 100 | 95 | 100 | 90 | 90 | 90 | 90 | 100 | 90 | 90 | 50 | 90 | 100 | 100 | 85 | 85 | 80 | 85 | 100 |
| Morningglory | — | 0 | 0 | 0 | 0 | 0 | 0 | 75 | 45 | 0 | 100 | 20 | 100 | 0 | — | 0 | 0 | 100 | 0 | 0 | 0 | 0 | 0 | 0 | 0 | 0 | 0 |
| Nightshade | 95 | 90 | 100 | 50 | 0 | 80 | 50 | 55 | 75 | 95 | 100 | 100 | 100 | 80 | 90 | 60 | 10 | — | 0 | 80 | 90 | 100 | 50 | 0 | 15 | 90 | 85 |
| Nutsedge, Yellow | 10 | 0 | 50 | 0 | 0 | 10 | 0 | 0 | 20 | 40 | 0 | 0 | 0 | 0 | 0 | 0 | 0 | 0 | 0 | 0 | 0 | 0 | 0 | 0 | 0 | 0 | 0 |
| Pigweed | 95 | 80 | 100 | 30 | 95 | 100 | 80 | 100 | 100 | 100 | 100 | 100 | 100 | 90 | 100 | 50 | 100 | 90 | 0 | 10 | 20 | 100 | 100 | 90 | 95 | 100 | 100 |
| Ragweed | 60 | 0 | 100 | 50 | 0 | 100 | 0 | 95 | 100 | 85 | 0 | 0 | — | 0 | 0 | 0 | 0 | 10 | 0 | 0 | 10 | 80 | 0 | 10 | 0 | 60 | 65 |
| Soybean | 20 | 0 | 70 | 0 | 0 | 0 | 0 | 0 | 15 | 0 | 0 | 0 | 30 | 0 | 0 | 0 | 0 | 0 | 0 | 0 | 0 | 0 | 0 | 0 | 0 | 0 | 0 |
| Sunflower | 0 | 0 | 0 | 0 | 0 | 0 | 0 | 0 | 60 | 0 | 10 | 0 | 50 | 10 | 0 | 0 | 0 | 10 | 0 | 0 | 0 | 0 | 0 | 0 | 0 | 0 | 0 |
| Surinam Grass | 50 | 0 | 85 | 0 | 0 | — | — | 45 | 90 | 65 | 20 | 20 | 10 | 0 | 10 | 20 | 10 | 10 | 20 | 10 | 10 | 0 | 0 | 0 | 0 | 10 | 0 |
| Velvetleaf | 80 | 30 | 40 | 0 | 25 | 0 | 10 | 85 | 90 | 100 | 40 | 20 | 80 | 0 | 20 | — | 0 | 100 | 40 | 70 | 90 | 40 | 0 | 0 | 0 | 20 | 0 |
| | 62 g ai/ha | | | | | | | | | | | | | | | | | | | | | | | | | | |
| | Compounds | | | | | | | | | | | | | | | | | | | | | | | | | | |
| | 58 | 60 | 61 | 62 | 63 | 64 | 65 | 80 | 88 | 95 | 96 | 102 | 108 | 114 | 115 | 118 | 128 | 130 | 131 | 134 | 137 | 139 | 140 | 144 | 145 | 146 | 147 |
| Bermudagrass | 90 | 80 | 10 | 10 | 50 | 0 | 40 | 10 | 90 | 70 | 40 | 100 | 100 | 50 | 100 | 90 | 100 | 60 | 15 | 80 | 50 | 60 | 0 | 70 | 20 | 10 | 40 |
| Cocklebur | 0 | 0 | 0 | 0 | 0 | 0 | 0 | 0 | 0 | 0 | 0 | 10 | 20 | 20 | 20 | 20 | 10 | 0 | 0 | 0 | 0 | 0 | 0 | 0 | 0 | 0 | 0 |
| Corn | 0 | 0 | 0 | 0 | 0 | 0 | 0 | 0 | 0 | 0 | 15 | 0 | 30 | 10 | 10 | 0 | 20 | 0 | 0 | 0 | 0 | 0 | 0 | 0 | 0 | 0 | 0 |
| Crabgrass, Large | 90 | 90 | 50 | 40 | 10 | 0 | 50 | 0 | 90 | 80 | 85 | 100 | 100 | 100 | 100 | 70 | 100 | — | 45 | 65 | 40 | 40 | 0 | 50 | — | 20 | 0 |
| Cupgrass, Woolly | 0 | 0 | 0 | 0 | 0 | 0 | 0 | 0 | 20 | 5 | 5 | 0 | 30 | — | 20 | 0 | 10 | 5 | 0 | 0 | 0 | 0 | 0 | 0 | 0 | 0 | 0 |
| Foxtail, Giant | 70 | 0 | 0 | 15 | 0 | 0 | 0 | 10 | 0 | 60 | 70 | 10 | 100 | 20 | 40 | 0 | 10 | 40 | 20 | 0 | 10 | 40 | 0 | 40 | 0 | 0 | 0 |
| Goosegrass | 90 | 55 | 0 | 10 | 10 | 0 | 0 | 10 | 60 | 0 | 30 | 90 | 90 | 40 | 70 | 20 | 80 | 10 | 15 | 20 | 40 | 60 | 0 | 30 | 50 | 0 | 0 |
| Johnsongrass | 70 | 10 | 10 | 40 | 30 | 0 | 10 | 10 | 0 | 0 | 40 | 50 | 60 | 20 | 70 | 40 | 90 | — | 15 | 0 | 0 | 20 | 0 | 0 | 50 | — | 0 |
| Kochia | 95 | 80 | 10 | 100 | 30 | 0 | 10 | 10 | 0 | 100 | 90 | 0 | 100 | 90 | 100 | 20 | 0 | 0 | 85 | 85 | 80 | 100 | 70 | 75 | 20 | 50 | — |
| Lambsquarters | 100 | 100 | 100 | 100 | 30 | 0 | 20 | 0 | 95 | — | — | 0 | 100 | 80 | 100 | 50 | — | 10 | 95 | 100 | 40 | 100 | 75 | 95 | 20 | 85 | 70 |
| Morningglory | 10 | 0 | 0 | — | 0 | 0 | 0 | 20 | 0 | 5 | 0 | 30 | 40 | — | — | 20 | 30 | 0 | 0 | 0 | 0 | 0 | 0 | 0 | 0 | 0 | 0 |
| Nightshade | 95 | 30 | 0 | 80 | 70 | 0 | 50 | 10 | 40 | 95 | 90 | 90 | 100 | 70 | 100 | 100 | 90 | 30 | 55 | 90 | 60 | 95 | 0 | 90 | 60 | 50 | 40 |
| Nutsedge, Yellow | 10 | 0 | 0 | 0 | 0 | 0 | 0 | 0 | 0 | 0 | 0 | 0 | 50 | 0 | 0 | 0 | — | 0 | 0 | 0 | 0 | 0 | 0 | 0 | 0 | 0 | 0 |
| Pigweed | 100 | 100 | 0 | 95 | 100 | 30 | 70 | 40 | 100 | 100 | 100 | 90 | 100 | 100 | 100 | 70 | — | 80 | 95 | 95 | 100 | 100 | 100 | 90 | 85 | 80 | 20 |
| Ragweed | 60 | 10 | 0 | 10 | 0 | 0 | 0 | 0 | 0 | 50 | 50 | 10 | 20 | 10 | 70 | 0 | 10 | 0 | 40 | 0 | 0 | 50 | 0 | 0 | 0 | 0 | 0 |
| Soybean | 0 | 0 | 0 | 0 | 0 | 0 | 0 | 0 | 0 | 0 | 0 | 0 | — | 70 | 0 | 0 | 20 | 0 | 0 | 0 | 0 | — | 0 | 0 | — | 0 | 0 |
| Sunflower | 0 | 0 | 0 | 0 | 0 | 0 | 0 | — | 0 | 0 | 0 | 0 | 20 | 30 | 0 | 0 | — | 0 | 0 | 0 | 0 | 0 | 0 | 0 | 0 | 0 | 0 |
| Surinam Grass | 10 | 0 | 0 | 5 | 0 | 0 | 0 | 0 | 10 | 0 | 0 | 0 | 30 | 10 | 20 | — | 30 | 5 | 0 | 0 | 0 | 0 | 0 | 20 | 0 | 0 | 0 |
| Velvetleaf | 80 | 5 | 0 | 40 | 10 | 0 | 0 | 20 | 0 | 0 | 50 | 0 | 60 | 30 | 20 | 0 | 30 | 0 | 0 | 20 | 0 | — | 0 | 20 | 0 | 0 | 5 |
| | 62 g ai/ha | | | | | | | | | | | | | | | | | | | | | | | | | | |
| | Compounds | | | | | | | | | | | | | | | | | | | | | | | | | | |
| | 149 | 152 | 153 | 156 | 162 | 182 | 188 | 193 | 195 | 198 | 199 | 202 | 205 | 207 | 210 | 212 | 213 | 215 | 221 | 222 | 223 | 242 | 244 | 268 | 269 | 287 | 293 |
| Bermudagrass | 30 | 85 | 70 | 88 | 0 | 20 | 75 | 60 | 0 | 60 | 55 | 20 | 90 | 50 | 80 | 60 | 50 | 40 | 0 | 85 | 80 | 95 | 95 | 100 | 100 | 85 | 90 |
| Cocklebur | 0 | — | 0 | 63 | — | 0 | 0 | 0 | 0 | 0 | 0 | 0 | 0 | 0 | 0 | 0 | — | 0 | 0 | 0 | 0 | 0 | 0 | 10 | 0 | 0 | 0 |
| Corn | 0 | 40 | 0 | 15 | 20 | 0 | 0 | 10 | 0 | 0 | 0 | 0 | 0 | 0 | 0 | 0 | 0 | 0 | 0 | 0 | 0 | 0 | 0 | 10 | — | — | — |
| Crabgrass, Large | 20 | 100 | 0 | 60 | 35 | 0 | 95 | 40 | 0 | 80 | 10 | 80 | 60 | 80 | 80 | 60 | 0 | 0 | 0 | 100 | 100 | 100 | 100 | 90 | 95 | 65 | 95 |
| Cupgrass, Woolly | 0 | 40 | 0 | 33 | 0 | 0 | 0 | 10 | 0 | 0 | 0 | 0 | 0 | 0 | 0 | 0 | 0 | 0 | 0 | 20 | 0 | 75 | 65 | 50 | 10 | 0 | 45 |
| Foxtail, Giant | 0 | 70 | 0 | 75 | 55 | 0 | 0 | 40 | 0 | 0 | 0 | 0 | 0 | 0 | 0 | 0 | 30 | 0 | 0 | 20 | 40 | 85 | 80 | 80 | 50 | 0 | 10 |
| Goosegrass | 10 | 80 | 55 | 83 | 60 | 0 | 15 | 60 | 100 | 20 | 5 | 20 | 50 | 10 | 50 | 10 | 0 | 0 | 0 | 80 | 60 | 100 | 70 | 85 | 70 | 70 | 65 |
| Johnsongrass | 10 | 80 | 0 | 80 | 65 | 0 | 0 | 60 | 0 | 0 | 0 | 0 | 0 | 0 | 5 | 0 | 0 | 0 | 0 | 85 | 80 | 65 | 60 | 70 | 40 | 55 | 65 |
| Kochia | 0 | 100 | 65 | 95 | 65 | 0 | 0 | 85 | 0 | 50 | 80 | 60 | 90 | 90 | 0 | 85 | — | 100 | 0 | 90 | 85 | — | — | 100 | 70 | 85 | 100 |
| Lambsquarters | 75 | 100 | 95 | 100 | 80 | 25 | 95 | 50 | 0 | 85 | 80 | 40 | 85 | 90 | 100 | 100 | 80 | 100 | 0 | 100 | 95 | — | — | 100 | 95 | 100 | 100 |
| Morningglory | 0 | 20 | 0 | 8 | 45 | 0 | 0 | 20 | 0 | 10 | — | 0 | 0 | 0 | 0 | 0 | 0 | 0 | 0 | 0 | 0 | 0 | 0 | 0 | 0 | — | — |
| Nightshade | 80 | 80 | 90 | 75 | 80 | 70 | 80 | 0 | 0 | 90 | 85 | 20 | 80 | 60 | 80 | 70 | 100 | 100 | 30 | 85 | 100 | 100 | 100 | 100 | 90 | 95 | 65 |
| Nutsedge, Yellow | 0 | 10 | 0 | 35 | 0 | 0 | 0 | 0 | 0 | 0 | 0 | 0 | 0 | 0 | 0 | 0 | 0 | 0 | 0 | 0 | 0 | 0 | 0 | 30 | 5 | 0 | 0 |
| Pigweed | 20 | 85 | 95 | 95 | 100 | 95 | 95 | 100 | 55 | 100 | 100 | 95 | 90 | 100 | 100 | 100 | 80 | 80 | 90 | 100 | 100 | 100 | 100 | 100 | 100 | 100 | 20 |
| Ragweed | 0 | 85 | 0 | 90 | 95 | 20 | 0 | 0 | 20 | 50 | 55 | 0 | 0 | 40 | 0 | 0 | 0 | 0 | 0 | 80 | — | 95 | 95 | 70 | 5 | 100 | 85 |
| Soybean | 0 | 0 | 0 | 33 | 15 | 0 | 0 | 30 | 0 | 0 | 0 | 0 | — | 0 | 0 | 0 | 0 | 0 | 0 | 40 | 0 | 0 | 0 | 60 | 0 | 0 | 0 |
| Sunflower | 0 | 0 | 0 | 25 | 0 | 0 | 0 | 0 | 0 | 0 | 0 | 0 | 0 | 0 | 0 | 0 | 0 | 0 | 0 | 0 | 0 | 0 | 0 | 0 | 0 | 90 | 10 |
| Surinam Grass | 0 | 40 | 0 | 75 | 0 | 0 | 20 | 50 | 0 | 0 | 0 | 10 | 0 | 0 | 0 | 0 | 0 | 0 | 0 | 80 | 50 | 85 | 100 | 70 | 5 | — | — |
| Velvetleaf | 0 | 0 | 0 | 43 | 70 | 0 | 0 | 0 | 0 | 5 | 10 | 5 | 10 | 0 | 0 | 20 | 0 | 0 | 0 | 50 | 0 | 100 | 15 | 60 | 5 | 40 | 80 |
| | 62 g ai/ha | | | | | | | | | | | | | | | | | | | | | | | | | | |
| | Compounds | | | | | | | | | | | | | | | | | | | | | | | | | | |
| | 294 | 298 | 299 | 300 | 301 | 304 | 305 | 306 | 307 | 309 | 310 | 314 | 318 | 332 | 333 | 334 | 336 | 340 | 341 | 342 | 343 | 352 | 372 | 375 | 376 | 382 | 384 |
| Bermudagrass | 70 | 50 | 100 | 95 | 80 | 5 | 80 | 40 | 90 | 90 | 50 | 10 | 10 | 80 | 40 | 100 | 95 | 20 | 80 | 45 | 60 | 95 | 75 | 100 | 80 | 60 | 100 |
| Cocklebur | 30 | 0 | 0 | 5 | 0 | 0 | 10 | 0 | 0 | 0 | 0 | 0 | 0 | 0 | 0 | 30 | 0 | 0 | 0 | 10 | 30 | 0 | 0 | 0 | 0 | 0 | — |
| Corn | 55 | 0 | — | 30 | 10 | 0 | 0 | 5 | 0 | 5 | — | 0 | 0 | 0 | 0 | 0 | 10 | 5 | 10 | 10 | 40 | 0 | 60 | 0 | 20 | 0 | — |
| Crabgrass, Large | 70 | 85 | 100 | 100 | 90 | 35 | 70 | 100 | 80 | — | 30 | 0 | — | 75 | 20 | 85 | 100 | 50 | 50 | 60 | 50 | 55 | 70 | 60 | 80 | 95 | 100 |
| Cupgrass, Woolly | 55 | 5 | 50 | 20 | 10 | 5 | 20 | 5 | 5 | 40 | 5 | 0 | 0 | 0 | 0 | 25 | 5 | 0 | 80 | 10 | 40 | 5 | 50 | 60 | 5 | 0 | 55 |
| Foxtail, Giant | — | 20 | 100 | 30 | 35 | 5 | 35 | 5 | 70 | 70 | 5 | 0 | 0 | 0 | 0 | 60 | 0 | 0 | 0 | 0 | 40 | 5 | 70 | 70 | 10 | 60 | 95 |
| Goosegrass | 80 | 85 | 100 | 85 | 50 | 45 | 80 | 50 | 90 | 85 | 70 | 0 | 0 | 80 | 0 | 100 | 95 | 5 | 70 | 60 | 60 | 80 | 70 | 60 | 80 | 75 | 85 |
| Johnsongrass | 80 | 20 | 85 | 60 | 30 | 0 | 65 | 40 | 60 | 75 | 0 | 0 | 0 | 0 | 0 | 90 | 80 | 15 | 15 | 55 | 70 | 50 | 80 | 0 | 60 | 70 | 100 |
| Kochia | 100 | 80 | 100 | 60 | 100 | — | 100 | 0 | 100 | 100 | 95 | 0 | 10 | 100 | 80 | 100 | 5 | 10 | 80 | 50 | 90 | 80 | 100 | 80 | 95 | 0 | 90 |
| Lambsquarters | 100 | 100 | 100 | 100 | 100 | 0 | 100 | 100 | 100 | 100 | 95 | 0 | 80 | 100 | 95 | 100 | 100 | 70 | 100 | 70 | 100 | 85 | 100 | 5 | 100 | 90 | 100 |
| Morningglory | 30 | — | 5 | 5 | 5 | 0 | 0 | 5 | 0 | 0 | 0 | 0 | 0 | 0 | 0 | 5 | 5 | 0 | 0 | 0 | 5 | 5 | 0 | 0 | 0 | 0 | — |
| Nightshade | 100 | 100 | 100 | 100 | 90 | 80 | 60 | 90 | 100 | 70 | 30 | 60 | 80 | 20 | — | 100 | 85 | 60 | 50 | 60 | 60 | 0 | 85 | 50 | 95 | 95 | — |
| Nutsedge, Yellow | 50 | 0 | 40 | 0 | 0 | 0 | 10 | 0 | 5 | 5 | 0 | 0 | 0 | 0 | 0 | 10 | 0 | 0 | 0 | 0 | 5 | 0 | 5 | 0 | 5 | 0 | — |
| Pigweed | 100 | 100 | 100 | 100 | 100 | 100 | 100 | 100 | 100 | 80 | 100 | 30 | 80 | 100 | 100 | 100 | 100 | 70 | 95 | 80 | 80 | 40 | 85 | 50 | 100 | 95 | 100 |
| Ragweed | 100 | 0 | 100 | 5 | 10 | 0 | 10 | 0 | 80 | 50 | 0 | 0 | 0 | 0 | 0 | 75 | 30 | 0 | 50 | 55 | 70 | 0 | 70 | 0 | 0 | 20 | 100 |
| Soybean | 70 | 0 | 55 | 0 | 15 | 0 | 0 | 5 | 40 | 20 | 0 | 0 | 0 | 0 | 0 | 15 | 0 | 0 | 0 | 10 | 40 | 0 | 0 | 0 | 0 | 100 | — |
| Sunflower | 30 | 0 | 10 | 0 | 0 | 0 | 0 | 0 | 0 | 0 | 0 | 0 | 0 | 0 | 0 | 5 | 0 | 0 | 0 | 5 | 10 | 0 | 0 | 0 | 0 | 0 | — |
| Surinam Grass | 85 | 5 | 75 | 30 | 0 | 0 | 50 | — | 70 | 50 | 5 | 0 | 10 | 5 | — | 55 | 50 | 0 | 60 | 40 | 60 | 10 | 50 | 55 | 40 | — | — |
| Velvetleaf | 100 | 60 | 5 | 5 | 20 | 0 | 40 | 5 | 70 | 5 | 0 | 0 | 60 | 40 | 0 | 5 | 5 | 0 | 30 | 50 | 60 | 0 | 50 | 0 | 0 | 100 | 100 |
| | 62 g ai/ha | | | | | | | | | | | | | | 31 g ai/ha | | | | | | | | | | | | |
| | Compounds | | | | | | | | | | | | | | Compounds | | | | | | | | | | | | |
| | 385 | 386 | 391 | 392 | 393 | 395 | 396 | 399 | 409 | 414 | 417 | 418 | 419 | 420 | 4 | 5 | 27 | 29 | 33 | 35 | 41 | 60 | 62 | 80 | 95 | 114 | 118 |
| Bermudagrass | 55 | 90 | 95 | 90 | 60 | 95 | 95 | 100 | 40 | 100 | 100 | 100 | 100 | 100 | 30 | 0 | 20 | 40 | 50 | 30 | 30 | 30 | 0 | 0 | 10 | 10 | 30 |
| Cocklebur | 0 | 0 | 20 | 0 | 0 | 0 | 0 | 15 | 0 | 0 | 0 | 0 | 0 | 0 | 0 | 0 | 0 | 30 | 0 | 0 | 0 | 0 | 0 | — | 0 | 0 | 0 |
| Corn | 0 | 10 | 80 | 30 | 55 | 0 | 0 | 60 | 0 | 0 | 0 | 0 | 0 | 0 | 0 | 0 | 0 | 0 | 0 | 0 | — | 0 | 0 | 0 | 0 | 10 | 0 |
| Crabgrass, Large | 90 | 98 | 98 | 98 | 95 | 100 | 100 | 100 | 85 | 100 | 100 | 95 | 100 | 100 | 40 | 0 | 100 | 90 | 90 | 0 | 0 | 80 | 5 | 0 | 10 | 100 | 10 |
| Cupgrass, Woolly | 0 | 50 | 70 | 30 | 30 | 85 | 60 | 45 | 40 | 0 | 0 | 0 | 0 | 0 | 0 | 0 | 0 | 0 | 20 | 10 | 0 | 0 | 0 | 0 | 0 | 0 | 0 |
| Foxtail, Giant | 40 | 80 | 85 | 65 | 70 | 95 | 95 | 95 | 30 | 75 | 65 | 65 | 75 | 20 | 0 | 0 | 10 | 0 | 10 | 0 | 0 | 0 | 5 | 10 | 0 | 20 | 0 |
| Goosegrass | 70 | 85 | 80 | 85 | 85 | 95 | 95 | 95 | 80 | 95 | 95 | 55 | 45 | 95 | 10 | 0 | 20 | 20 | 20 | 20 | 10 | 10 | 0 | 10 | 0 | 20 | 20 |
| Johnsongrass | 80 | 85 | 100 | 85 | 95 | 95 | 95 | 95 | 35 | 95 | 100 | 95 | 100 | 100 | 5 | 0 | 20 | 20 | 20 | 10 | 0 | 5 | 0 | 0 | 0 | 0 | 30 |
| Kochia | 0 | 30 | 100 | 75 | 0 | 100 | 0 | — | — | 60 | 0 | 65 | 90 | 70 | 10 | 0 | 20 | 0 | 80 | 0 | 0 | 80 | 100 | 10 | 80 | 90 | 10 |
| Lambsquarters | 75 | 100 | 100 | 100 | 100 | 100 | 100 | 100 | 100 | 95 | 95 | 95 | 95 | 95 | 90 | 0 | 90 | 80 | 80 | 0 | 0 | 90 | 100 | 0 | — | 40 | 50 |
| Morningglory | 15 | 55 | 100 | 70 | 60 | 15 | 0 | 15 | 10 | 10 | 0 | 20 | 15 | 10 | — | 0 | 90 | 90 | 0 | 0 | 0 | 0 | — | 0 | 0 | 40 | 0 |
| Nightshade | — | 90 | 90 | 90 | — | 100 | 95 | 95 | 45 | 85 | 55 | 95 | 85 | 80 | 90 | 80 | 90 | 90 | 10 | 70 | 0 | 10 | 80 | 0 | 90 | 20 | 60 |
| Nutsedge, Yellow | 100 | 0 | 5 | 0 | — | 15 | 0 | 45 | 0 | 0 | 0 | 0 | 0 | 0 | 0 | 0 | 0 | 0 | 0 | 0 | 0 | 0 | 0 | 0 | 0 | 0 | 0 |
| Pigweed | 80 | 100 | 95 | 100 | 90 | 100 | 100 | 100 | 100 | 100 | 100 | 100 | 100 | 100 | 90 | 40 | 100 | 80 | 30 | 0 | 0 | 100 | 90 | 30 | 100 | 0 | 20 |
| Ragweed | 0 | 5 | 85 | 60 | 20 | 65 | 0 | 95 | 45 | 15 | 0 | 50 | 10 | 0 | 50 | 0 | 0 | 0 | 0 | 0 | 0 | 0 | 0 | 0 | 10 | 10 | 0 |
| Soybean | 25 | 90 | 95 | 5 | — | 75 | 0 | 75 | 0 | 0 | 0 | 0 | 40 | 0 | 0 | 0 | 0 | 30 | 0 | 0 | 0 | 0 | 0 | 0 | 0 | 60 | 0 |
| Sunflower | — | — | 50 | 10 | — | 0 | 0 | 0 | 0 | 0 | 0 | 0 | 0 | 0 | 0 | 0 | 0 | 30 | 0 | 10 | 0 | 0 | 0 | 0 | 0 | 30 | 0 |
| Surinam Grass | — | 80 | 85 | 50 | — | 95 | 65 | 95 | 50 | 0 | 0 | 50 | 0 | 10 | 0 | 0 | 20 | 0 | 20 | 0 | 10 | 0 | 0 | 0 | 0 | 10 | 20 |
| Velvetleaf | 50 | 70 | 100 | 70 | 65 | 95 | 0 | 95 | 5 | 90 | 75 | 90 | 100 | 80 | 5 | 0 | 0 | 10 | 0 | 0 | 0 | 5 | 0 | 20 | 0 | 0 | 0 |

| | 31 g ai/ha | | | | | | | | 16 g ai/ha | | | | | | |
|---|---|---|---|---|---|---|---|---|---|---|---|---|---|---|---|
| | Compounds | | | | | | | | Compounds | | | | | | |
| | 128 | 131 | 139 | 156 | 300 | 334 | 375 | 384 | 4 | 5 | 33 | 35 | 118 | 128 | 156 |
| Bermudagrass | 90 | 0 | 10 | 60 | 80 | 100 | 95 | 65 | 0 | 0 | 30 | 0 | 0 | 30 | 0 |
| Cocklebur | 10 | 0 | 0 | 40 | 0 | 5 | 0 | — | 0 | 0 | 0 | 0 | 0 | 10 | — |
| Corn | 20 | 0 | 0 | 0 | 0 | 0 | | | 0 | 0 | 0 | 0 | 0 | 20 | 0 |
| Crabgrass, Large | 10 | 0 | 20 | 60 | 100 | 70 | 30 | 80 | 0 | 0 | 0 | 0 | 0 | 10 | 30 |
| Cupgrass, Woolly | 10 | 0 | 0 | 20 | 5 | 20 | 30 | 0 | 0 | 0 | 20 | 10 | 0 | 0 | 0 |
| Foxtail, Giant | 0 | 0 | 0 | 40 | 10 | 30 | 60 | 20 | 0 | 0 | 0 | 0 | 0 | 0 | 0 |
| Goosegrass | 20 | 0 | 0 | 70 | 70 | 85 | 40 | 20 | 5 | 0 | 10 | 0 | 20 | 10 | 50 |
| Johnsongrass | 80 | 0 | 0 | 70 | 40 | 80 | 0 | 95 | 0 | 0 | 20 | 10 | 30 | 0 | 5 |
| Kochia | 0 | 85 | 70 | 90 | 60 | 100 | 20 | 75 | 0 | 0 | 70 | 0 | 10 | 0 | 80 |
| Lambsquarters | 10 | 95 | 100 | 100 | 80 | 100 | 0 | 100 | 50 | 0 | 0 | 0 | 40 | 0 | 85 |
| Morningglory | 30 | 0 | 0 | 0 | 5 | 0 | 0 | — | — | — | 0 | 0 | 0 | 20 | 0 |
| Nightshade | 70 | 0 | 70 | — | 90 | 100 | 5 | — | — | 80 | 10 | 20 | 20 | 50 | 30 |
| Nutsedge, Yellow | 70 | 0 | 0 | 10 | 0 | 5 | 0 | — | 0 | 0 | 0 | 0 | 0 | 10 | 0 |
| Pigweed | 50 | 95 | 100 | 80 | 100 | 85 | — | 95 | 90 | 0 | 10 | 0 | 20 | 0 | 80 |
| Ragweed | 0 | 0 | 0 | — | 5 | 75 | 0 | 100 | 0 | 0 | 0 | 0 | 0 | 0 | 0 |
| Soybean | 20 | 0 | 0 | 20 | 0 | 10 | 0 | — | 0 | 0 | 0 | 0 | 0 | 20 | 0 |
| Sunflower | 50 | 0 | 0 | 0 | 0 | 0 | 0 | — | 0 | 0 | 0 | 10 | 0 | 50 | 0 |
| Surinam Grass | 20 | 0 | 0 | 40 | 5 | 50 | 20 | — | 0 | 0 | 20 | 0 | 20 | 0 | 0 |
| Velvetleaf | 30 | 0 | 0 | 20 | 5 | 0 | 0 | 65 | 0 | 0 | 0 | 0 | 0 | 20 | 0 |

Test D

Three plastic pots (ca. 16-cm diameter) per rate were partially filled with sterilized Tama silt loam soil comprising a 35:50:15 ratio of sand, silt and clay and 2.6% organic matter. Separate plantings for each of the three pots were as follows. Seeds from the U.S. of ducksalad (Heteranthera limosa (Sw.) Willd.), smallflower umbrella sedge (Cyperus difformis L.) and purple redstem (Ammannia coccinea Rottb.), were planted into one 16-cm pot for each rate. Seeds from the U.S. of rice flatsedge (Cyperus iria L.), bearded sprangletop (Leptochloa fascicularis (Lam.) Gray), one stand of 9 or 10 water seeded rice seedlings (Oryza sativa L. cv. ‘Japonica—M202’), and one stand of 6 transplanted rice seedlings (Oryza sativa L. cv. ‘Japonica—M202’) were planted into one 16-cm pot for each rate. Seeds from the U.S. of barnyardgrass (Echinochloa crus-galli (L.) Beauv.), late watergrass (Echinochloa oryzicola Vasinger), early watergrass (Echinochloa oryzoides (Ard.) Fritsch) and junglerice (Echinochloa colona (L.) Link) were planted into one 16-cm pot for each rate. Plantings were sequential so that crop and weed species were at the 2.0 to 2.5-leaf stage at time of treatment.

Potted plants were grown in a greenhouse with day/night temperature settings of 29.5/26.7° C., and supplemental balanced lighting was provided to maintain a 16-hour photoperiod. Test pots were maintained in the greenhouse until test completion.

At time of treatment, test pots were flooded to 3 cm above the soil surface, treated by application of test compounds directly to the paddy water, and then maintained at that water depth for the duration of the test. Effects of treatments on rice and weeds were visually evaluated by comparison to untreated controls after 21 days. Plant response ratings are reported on a 0 to 100 scale; where 0 is no effect and 100 is complete control. A dash (-) response means no test result.

TABLE D
Flooded Paddy

| | Rate 1000 | | Rate 750 | | Rate | | | Rate | | | Rate | | | |
|---|---|---|---|---|---|---|---|---|---|---|---|---|---|---|
| | g ai/ha | | g ai/ha | | 500 g ai/ha | | | 250 g ai/ha | | | 125 g ai/ha | | | |
| | Compounds | | Compounds | | Compounds | | | Compounds | | | Compounds | | | |
| | 3 | 215 | 3 | 215 | 3 | 155 | 215 | 3 | 155 | 215 | 3 | 152 | 155 | 215 |
| Barnyardgrass | 90 | 40 | 60 | 30 | 20 | 80 | 0 | 0 | 35 | 0 | 0 | 35 | 20 | 0 |
| Ducksalad | 100 | 75 | 100 | 20 | 95 | 60 | 20 | 85 | 60 | 0 | 30 | 90 | 0 | 0 |
| Flatsedge, Rice | 100 | 90 | 100 | 80 | 100 | 70 | 60 | 100 | 60 | 60 | 70 | 95 | 30 | 0 |
| Junglerice | 100 | 25 | 100 | 25 | 70 | 65 | 20 | 30 | 40 | 0 | 20 | 30 | 40 | 0 |
| Redstem | 100 | 100 | 100 | 100 | 100 | 95 | 100 | 85 | 95 | 100 | 40 | 90 | 0 | 0 |
| Rice, Transplanted | 60 | 35 | 60 | 20 | 50 | 30 | 20 | 30 | 30 | 10 | 20 | 45 | 20 | 0 |
| Rice, Water Seeded | 80 | 45 | 70 | 45 | 60 | 40 | 45 | 45 | 35 | 35 | 40 | 45 | 25 | 20 |
| Sedge, Umbrella | 100 | 100 | 100 | 95 | 100 | 80 | 95 | 95 | 75 | 95 | 80 | 95 | 75 | 20 |
| Sprangletop, Bearded | 85 | 20 | 85 | 20 | 60 | 75 | 20 | 20 | 40 | 20 | 0 | 40 | 30 | 20 |
| Watergrass, Early | 60 | 40 | 45 | 25 | 40 | 70 | 20 | 20 | 35 | 0 | 20 | 45 | 25 | 0 |
| Watergrass, Late | 80 | 20 | 60 | 20 | 60 | 60 | 20 | 20 | 30 | 0 | 0 | 40 | 20 | 0 |

| | Rate | | Rate | | Rate 16 | Rate |
|---|---|---|---|---|---|---|
| | 64 g ai/ha | | 32 g ai/ha | | g ai/ha | 8 g ai/ha |
| | Compound | | Compound | | Compound | Compound |
| | 152 | 155 | 152 | 155 | 152 | 152 |
| Barnyardgrass | 10 | 0 | 0 | 0 | 0 | 0 |
| Ducksalad | 80 | 0 | 75 | 0 | 70 | 0 |
| Flatsedge, Rice | 90 | 0 | 75 | 0 | 75 | 75 |
| Junglerice | 0 | 40 | 0 | 20 | 0 | 0 |
| Redstem | 85 | 0 | 60 | 0 | 0 | 0 |
| Rice, Transplanted | 40 | 0 | 25 | 0 | 20 | 0 |
| Rice, Water Seeded | 40 | 0 | 30 | 0 | 20 | 0 |
| Sedge, Umbrella | 85 | 65 | 75 | 30 | 70 | 0 |
| Sprangletop, Bearded | 35 | 30 | 30 | 30 | 30 | 30 |
| Watergrass, Early | 0 | 0 | 0 | 0 | 0 | 0 |
| Watergrass, Late | 20 | 0 | 15 | 0 | 0 | 0 |

Test E

Seeds of plant species selected from bipinnate beggarticks (Bidens bipinnata L.), hairy beggarticks (Bidens radiata Thuill.), bermudagrass (Cynodon dactylon (L.) Pers.), Surinam grass (Urochloa decumbens (Staph) R. D. Webster, previously named Brachiaria decumbens Stapf), large crabgrass (Digitaria sanguinalis (L.) Scop.), green foxtail (Setaria viridis (L.) P. Beauv.), goosegrass (Eleusine indica (L.) Gaertn.), johnsongrass (Sorghum halepense (L.) Pers.), kochia (Kochia scoparia (L.) Schrad.), pitted morningglory (Ipomoea lacunosa L.), purple nutsedge (Cyperus rotundus L.), common ragweed (Ambrosia elatior L.), mustard (Brassica nigra (L.) W. D. J. Koch), guineagrass (Panicum maximum Jacq.), dallisgrass (Paspalum dilatatum Poir.), barnyardgrass (Echinochloa crus-galli (L). P. Beauv.), southern sandbur (Cenchrus echinatus L.), common sowthistle (Sonchus oleraceous L.), prickly sida (Sida spinosa L.), Italian ryegrass (Lolium multiflorum Lam.), common purslane (Portulaca oleracea L.), broadleaf signalgrass (Brachiaria platyphylla (Griseb.) Nash), common groundsel (Senecio vulgaris L.), common chickweed (Stellaria media (L.) Vill/Cyr.), tropical spiderwort (Commelina benghalensis L.), annual bluegrass (Poa annua L.), downy bromegrass (Bromus tectorum L.), itchgrass (Rottboellia cochinchinensis (L.) L.f.), quackgrass (Elytrigia repens (L.) Nevski), Canada horseweed (Erigeron Canadensis L.), field bindweed (Convolvulus arvensis L.), spotted spurge (Euphorbia maculata L.), common mallow (Malva sylvestris (=s silvestris) L.), and Russian thistle (Salsola kali L. ssp. Ruthenica (Iljin) Soo) were planted and treated preemergence with test chemicals formulated in a non-phytotoxic solvent mixture which included a surfactant.

At the same time, plants selected from these weed species were treated with postemergence applications of some of the test chemicals formulated in the same manner. Plants ranged in height from 2 to 18 cm (1- to 4-leaf stage) for postemergence treatments. Treated plants and controls were maintained in a greenhouse for 12 to 14 days, after which time all species were compared to controls and visually evaluated. Plant response ratings, summarized in Table E, are based on a scale of 0 to 100 where 0 is no effect and 100 is complete control. A dash (-) response means no test result.

TABLE E
Postemergence

| | 500 g ai/ha | | | | | | | | | | 250 g ai/ha | | | | | | | | | | | | 125 g ai/ha | | | | |
|---|---|---|---|---|---|---|---|---|---|---|---|---|---|---|---|---|---|---|---|---|---|---|---|---|---|---|---|
| | Compounds | | | | | | | | | | Compounds | | | | | | | | | | | | Compounds | | | | |
| | 6 | 14 | 16 | 152 | 156 | 162 | 193 | 222 | 294 | 299 | 2 | 6 | 13 | 14 | 16 | 152 | 156 | 162 | 193 | 222 | 294 | 299 | 2 | 6 | 13 | 14 | 16 |
| Barnyardgrass | 98 | 75 | 80 | 75 | 98 | 95 | 90 | — | 80 | 80 | 95 | 95 | 70 | 50 | 70 | 75 | 98 | 90 | 80 | 70 | 75 | 70 | 90 | 90 | 70 | 30 | 60 |
| Beggarticks, Bip. | — | — | — | — | — | — | — | — | 75 | 80 | — | — | — | — | — | — | — | — | — | — | 70 | 60 | — | — | — | — | — |
| Bermudagrass | 98 | 85 | 80 | 75 | 98 | 80 | 95 | 80 | 75 | 80 | 90 | 90 | 90 | 50 | 80 | 75 | 95 | 70 | 90 | 80 | 75 | 70 | 85 | 80 | 50 | 30 | 40 |
| Bindweed, Field | 100 | 60 | 40 | 50 | 100 | 98 | 50 | — | 70 | 95 | 70 | 100 | 30 | 50 | 30 | 30 | 80 | 95 | 50 | 95 | 50 | 80 | 70 | 95 | 20 | 30 | 10 |
| Black Mustard | 100 | 100 | 100 | 100 | 100 | 95 | 100 | 95 | 95 | 100 | 100 | 100 | 90 | 80 | 100 | 100 | 100 | 90 | 95 | 95 | 90 | 100 | 95 | 100 | 80 | — | 100 |
| Bluegrass | 98 | 90 | 100 | 85 | 98 | 98 | 85 | 90 | 95 | 100 | 100 | 98 | 60 | 75 | 80 | 85 | 98 | 98 | 70 | 90 | 95 | 80 | 98 | 98 | 30 | 50 | 20 |
| Bromegrass, Downy | 100 | — | 100 | 80 | 100 | 100 | 80 | — | 95 | 95 | 98 | 100 | 30 | 30 | 100 | 75 | 100 | 98 | 70 | 80 | 85 | 80 | 75 | 98 | 20 | 10 | — |
| Chickweed | 100 | — | 100 | 100 | 100 | — | 100 | 100 | — | — | — | 100 | 95 | 75 | 95 | — | 100 | 100 | 95 | 100 | — | — | 100 | 100 | 95 | — | — |
| Crabgrass, Large | 98 | 80 | 75 | 80 | 98 | 95 | 100 | 100 | 80 | 95 | 98 | 98 | 90 | 70 | 60 | 75 | 95 | 90 | 100 | 90 | 75 | 95 | 95 | 90 | 70 | 30 | 50 |
| Dallisgrass | 90 | 70 | 85 | 75 | 90 | 85 | 90 | 85 | 70 | 85 | 90 | 80 | 70 | 50 | 60 | 75 | 90 | 70 | 90 | 85 | 60 | 75 | 80 | 75 | 40 | 10 | 50 |
| Foxtail, Green | 98 | 80 | 85 | 85 | 100 | 95 | 90 | — | 85 | 100 | 95 | 98 | 60 | 50 | 60 | 85 | 100 | 85 | 50 | 80 | 80 | 85 | 90 | 90 | 20 | 20 | 40 |
| Goosegrass | 98 | 80 | 80 | 75 | 98 | 98 | 85 | 90 | 85 | 85 | 98 | 98 | 50 | 60 | 70 | 60 | 98 | 95 | 80 | 90 | 80 | 80 | 95 | 98 | — | 40 | 50 |
| Groundsel | 100 | 100 | 75 | 95 | 100 | 100 | 100 | 100 | — | — | 80 | 100 | 85 | — | 75 | 95 | 100 | 80 | 70 | 95 | 90 | 100 | — | 95 | 50 | — | 75 |
| Guineagrass | 98 | 100 | 70 | 80 | 98 | 98 | 100 | — | 85 | 85 | 95 | 95 | 70 | 85 | 50 | 70 | 95 | 90 | 70 | 100 | 80 | 75 | 75 | 90 | 40 | 30 | 50 |
| Horseweed | 100 | — | — | — | 100 | 100 | 90 | 100 | — | — | — | 100 | 100 | 95 | — | — | 100 | 100 | 70 | 90 | — | — | — | 100 | 40 | — | — |
| Itchgrass | 95 | 70 | 95 | 75 | 98 | 95 | 75 | — | 80 | 80 | 90 | 95 | 75 | 50 | 60 | 75 | 95 | 95 | 60 | 70 | 75 | 75 | 90 | 90 | 50 | 20 | 50 |
| Johnsongrass | 98 | 70 | 100 | 95 | 100 | 100 | 80 | — | 90 | 85 | 100 | 98 | 30 | 50 | 70 | 80 | 100 | 100 | 70 | 60 | 80 | 85 | 90 | 95 | 20 | 20 | 50 |
| Kochia | 100 | — | — | — | 100 | — | — | — | — | — | — | 100 | — | — | — | — | 100 | — | — | — | — | — | 100 | 100 | — | — | — |
| Mallow | 100 | — | 70 | 50 | 100 | 100 | 90 | — | 100 | 100 | 80 | 100 | 50 | 70 | 40 | 50 | 95 | 80 | 90 | 60 | 50 | 50 | 75 | 95 | 20 | — | — |
| Morningglory | 80 | 40 | 30 | 30 | 90 | 90 | 75 | 90 | 20 | 60 | 90 | 60 | 60 | 30 | 10 | 20 | 80 | 80 | 60 | 60 | 20 | 40 | 60 | 10 | 50 | 10 | 10 |
| Nutsedge, Purple | — | 70 | 75 | 75 | — | — | 70 | — | 90 | 85 | — | — | 40 | 40 | 75 | 60 | 90 | 75 | 40 | 30 | 80 | 80 | — | 95 | 20 | 30 | 50 |
| Prickly Sida | — | 75 | 100 | 85 | — | — | 95 | 100 | 100 | 95 | — | — | 85 | 60 | 70 | 60 | — | — | 75 | 100 | 80 | 95 | — | — | 70 | 60 | 60 |
| Purslane | 100 | — | — | — | 100 | — | 85 | — | 90 | 100 | — | 100 | 70 | — | — | — | 100 | — | 60 | 60 | 90 | 80 | — | 90 | 70 | — | — |
| Quackgrass | 98 | 60 | 100 | 80 | 98 | 95 | 80 | 85 | 85 | 100 | 95 | 95 | 60 | 50 | 95 | 80 | 95 | 90 | 80 | 80 | 85 | 85 | 90 | 90 | 30 | 20 | 50 |
| Ragweed | 95 | 70 | 80 | 75 | 98 | 90 | 75 | — | 80 | 90 | — | 95 | 60 | 60 | 60 | 60 | 90 | 80 | 70 | 70 | 70 | 70 | 80 | 90 | 30 | 20 | 20 |
| Ryegrass, Italian | 98 | 100 | 100 | 90 | 98 | 100 | 95 | 85 | 95 | 85 | 98 | 98 | 75 | 60 | 95 | 90 | 98 | 98 | 95 | 85 | 95 | 85 | 90 | 95 | 40 | 50 | 80 |
| Sandbur | 100 | 50 | 75 | 100 | 98 | 90 | 20 | 80 | 80 | 80 | 95 | 100 | 10 | 10 | 60 | 40 | 80 | 80 | 10 | 50 | 75 | 70 | 75 | 95 | 10 | 10 | 30 |
| Signalgrass | 98 | 75 | 70 | 70 | 98 | 90 | 80 | 85 | 70 | 80 | 85 | 95 | 50 | 50 | 40 | 60 | 95 | 80 | 75 | 80 | 50 | 70 | 80 | 90 | 10 | 10 | 20 |
| Sowthistle | 100 | 100 | 95 | 100 | 100 | 100 | 95 | 100 | 80 | — | 95 | 100 | 100 | 90 | 95 | 90 | 100 | 75 | 95 | 100 | — | 95 | 90 | 95 | 60 | 90 | 70 |
| Spiderwort | 95 | 80 | 30 | 80 | 65 | 65 | 70 | — | 90 | 90 | 70 | 90 | 30 | 30 | — | 10 | 50 | 60 | 50 | 60 | 70 | 80 | — | 75 | 30 | — | — |
| Spurge, Spotted | — | 100 | — | 95 | — | — | — | — | — | — | — | — | — | — | 95 | — | — | — | — | — | — | — | — | — | — | 20 | 95 |
| Surinam Grass | 98 | 80 | 75 | 80 | 100 | 100 | 90 | — | 85 | 90 | 100 | 95 | 80 | 20 | 60 | 60 | 98 | 98 | 85 | 80 | 80 | 75 | 90 | 90 | 30 | 20 | 60 |
| Thistle | — | 60 | 75 | 80 | — | — | — | — | 80 | 90 | — | — | — | — | 70 | 80 | 80 | — | — | — | 80 | 70 | — | — | — | 10 | 60 |

| | | |
|---|---|---|
| 125 g ai/ha | 62 g ai/ha | 31 g ai/ha |
| Compounds | Compounds | Compounds |

| | 152 | 156 | 162 | 193 | 222 | 294 | 299 | 2 | 6 | 13 | 156 | 162 | 193 | 222 | 294 | 299 | 13 |
|---|---|---|---|---|---|---|---|---|---|---|---|---|---|---|---|---|---|
| Barnyardgrass | 50 | 95 | 80 | 75 | 50 | 60 | 70 | 80 | 80 | 10 | 90 | 60 | 10 | 50 | 40 | 40 | 0 |
| Beggarticks, Bip. | — | — | — | — | — | 60 | 60 | — | — | — | — | — | — | — | 40 | 40 | |
| Bermudagrass | 40 | 95 | 30 | 90 | 80 | 50 | 50 | 20 | 35 | 50 | 65 | 0 | 60 | 60 | 20 | 40 | 20 |
| Bindweed, Field | 30 | 65 | 80 | — | 75 | 50 | 70 | 50 | 10 | 10 | 65 | 50 | 20 | 60 | 10 | 30 | 10 |
| Black Mustard | — | 100 | 75 | 95 | 90 | 90 | 100 | 90 | 95 | 70 | 98 | 60 | 60 | 90 | 70 | 100 | 70 |
| Bluegrass | 60 | 95 | 95 | 50 | 80 | 95 | 70 | 90 | 98 | 10 | 90 | 90 | 20 | 60 | 95 | 60 | 10 |
| Bromegrass, Downy | — | 100 | 95 | 30 | 50 | 75 | 70 | 65 | 95 | 10 | 90 | 50 | 20 | 10 | 60 | 50 | 0 |
| Chickweed | — | 100 | 100 | — | 100 | — | — | — | 100 | 50 | 100 | 80 | 40 | 80 | — | — | |
| Crabgrass, Large | 60 | 80 | 50 | 80 | 80 | 70 | 85 | 90 | 75 | 50 | 35 | 30 | 10 | 60 | 40 | 75 | 30 |
| Dallisgrass | 30 | 85 | 40 | 40 | 60 | 40 | 70 | 75 | 60 | 10 | 75 | 20 | 10 | 30 | 10 | 50 | 0 |
| Foxtail, Green | 40 | 95 | 50 | 20 | 40 | 60 | 70 | 60 | 80 | 10 | 75 | 30 | 10 | 10 | 40 | 30 | 10 |
| Goosegrass | 50 | 98 | 80 | 70 | 75 | 60 | 80 | 95 | 90 | 50 | 95 | 50 | 20 | 60 | 50 | 60 | 10 |
| Groundsel | 90 | 100 | 30 | 40 | 70 | — | 70 | — | 20 | 30 | 80 | 20 | 20 | 40 | 50 | — | 20 |
| Guineagrass | — | 90 | 75 | 60 | 70 | 70 | 70 | 30 | 80 | 40 | 60 | 35 | 20 | 70 | 50 | 60 | 40 |
| Horseweed | — | — | 70 | 50 | 40 | — | — | — | 75 | — | — | 70 | 50 | 20 | — | — | 10 |
| Itchgrass | 60 | 95 | 85 | 60 | 60 | 70 | 70 | 70 | 90 | 50 | 80 | 75 | 30 | 60 | 50 | 30 | 30 |
| Johnsongrass | 70 | 95 | 80 | 70 | 60 | 70 | 80 | 80 | 90 | 0 | 85 | 65 | 40 | 10 | 40 | 60 | 0 |
| Kochia | — | 100 | — | — | — | — | — | — | 100 | — | — | — | — | — | — | — | |
| Mallow | — | 90 | 60 | 60 | 60 | 40 | 30 | 50 | 65 | 10 | 80 | 40 | 20 | 50 | 20 | 10 | 0 |
| Morningglory | 10 | 65 | 70 | 60 | 40 | 10 | 20 | — | 0 | 20 | 10 | 30 | 30 | 20 | 0 | 10 | 10 |
| Nutsedge, Purple | 30 | 75 | — | 20 | 10 | 70 | 75 | — | 75 | 0 | — | — | 0 | 10 | 50 | 40 | 0 |
| Prickly Sida | — | — | — | 60 | 100 | 70 | 95 | — | — | 60 | — | — | 60 | 90 | 50 | 60 | 40 |
| Purslane | — | 100 | — | 60 | 30 | 60 | 75 | — | 75 | 30 | 75 | — | 20 | 10 | 10 | 70 | 20 |
| Quackgrass | 70 | 95 | 70 | 70 | 75 | 80 | 75 | 70 | 80 | 0 | 75 | 50 | 0 | 30 | 60 | 20 | 0 |
| Ragweed | 50 | 90 | 75 | 30 | 60 | 60 | 60 | 60 | 75 | 20 | 80 | 50 | 10 | 20 | 30 | 30 | 20 |
| Ryegrass, Italian | 90 | 95 | 95 | 60 | 80 | 80 | 80 | 80 | 80 | 10 | 90 | 50 | 30 | 50 | 70 | 60 | 0 |
| Sandbur | 20 | 75 | 50 | 0 | 10 | 50 | 40 | 65 | 85 | 0 | 0 | 20 | 0 | 10 | 10 | 30 | 0 |
| Signalgrass | 20 | 85 | 70 | 50 | 40 | 30 | 60 | 30 | 35 | 10 | 40 | 30 | 10 | 10 | 30 | 20 | 10 |
| Sowthistle | — | 90 | 75 | 60 | 60 | 60 | 85 | 90 | 75 | 50 | 90 | 75 | 10 | 20 | 60 | 30 | 10 |
| Spiderwort | — | 30 | 10 | 20 | 40 | 30 | 80 | 20 | 60 | — | 20 | 0 | — | — | 20 | 10 | 10 |
| Spurge, Spotted | — | — | — | — | — | — | — | | | | | | | | | | |
| Surinam Grass | 30 | 95 | 90 | 70 | 80 | 70 | 75 | 75 | 75 | 20 | 80 | 50 | 10 | 50 | 30 | 70 | 20 |
| Thistle | 70 | 80 | — | — | — | 60 | 70 | — | — | — | — | — | — | — | 30 | 40 | |

Preemergence

| | 500 g ai/ha | | | | | | | | | | | | | | | | | | | | | 375 g ai/ha | | 250 g ai/ha | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | Compounds | | Compounds | | | |
| | 2 | 6 | 7 | 14 | 16 | 144 | 152 | 156 | 162 | 193 | 222 | 268 | 293 | 294 | 299 | 372 | 384 | 391 | 392 | 393 | 395 | 293 | 294 | 2 | 6 | 7 | 13 |
| Barnyardgrass | 100 | 100 | 70 | 95 | 95 | 90 | 90 | 100 | 100 | 95 | 100 | 100 | 100 | 100 | 100 | 95 | 100 | 100 | 100 | 80 | 95 | 100 | 100 | 100 | 95 | 60 | 90 |
| Beggarticks, Hairy | — | — | 40 | — | — | — | — | — | — | — | — | 100 | 100 | 100 | 100 | | | | | | | 100 | 100 | — | — | 20 | — |
| Beggarticks, Bip. | | | | | | | | | | | | | | | | 60 | — | 100 | 100 | 100 | 100 | | | | | | |
| Bermudagrass | 100 | 100 | 80 | 95 | 100 | 90 | 95 | 100 | 100 | 100 | 100 | 100 | 95 | 95 | 95 | 90 | 95 | 95 | 90 | 100 | 95 | 95 | 95 | 100 | 95 | 80 | 100 |
| Bindweed, Field | 80 | 100 | 30 | 100 | 75 | 80 | 90 | 100 | 100 | 85 | 95 | 70 | 95 | 100 | 100 | 60 | 95 | 95 | 100 | 70 | 70 | 95 | 95 | 70 | — | 10 | 60 |
| Black Mustard | 100 | 100 | 100 | 100 | 100 | 100 | 100 | 100 | 100 | 100 | 100 | 100 | 100 | 100 | 95 | 100 | 100 | 100 | 100 | 95 | 95 | 100 | 100 | 100 | 100 | 50 | 100 |
| Bluegrass | 100 | 100 | 80 | 100 | 100 | 100 | 95 | 100 | 100 | 100 | 100 | 95 | 100 | 100 | 100 | 95 | 95 | 95 | 100 | 95 | 95 | 95 | 95 | 100 | — | 75 | 90 |
| Bromegrass, Downy | 90 | 100 | 60 | 90 | 95 | 80 | 90 | 100 | 100 | 90 | 100 | 100 | 80 | 100 | 100 | 85 | 90 | 95 | 30 | 80 | 95 | — | 100 | 90 | 95 | 60 | 50 |
| Chickweed | 95 | 100 | — | 95 | 100 | 95 | 100 | 100 | 95 | 100 | 100 | — | — | — | | 95 | 100 | 100 | 95 | 100 | 100 | | | 95 | 95 | — | 95 |
| Crabgrass, Large | 100 | 100 | 100 | 100 | 100 | 60 | 100 | 95 | 100 | 100 | 100 | 95 | 100 | 100 | 100 | 100 | 100 | 95 | 95 | 100 | 100 | 100 | 100 | 100 | — | 75 | 100 |
| Dallisgrass | 95 | 100 | 85 | 95 | 95 | 80 | 95 | 100 | 95 | 100 | 100 | 85 | 100 | 95 | 100 | 85 | 90 | 95 | 100 | 85 | 90 | 95 | 95 | 95 | 95 | 80 | 80 |
| Foxtail, Green | 100 | 100 | 50 | 100 | 100 | 90 | 100 | 100 | 100 | 100 | 100 | 100 | 100 | 100 | 100 | 70 | 95 | 100 | 95 | 90 | 90 | 95 | 100 | 100 | — | 30 | 95 |
| Goosegrass | 95 | 100 | 70 | 95 | 85 | 80 | 90 | 100 | 95 | 95 | 95 | 95 | 100 | 100 | 100 | 75 | 95 | 95 | 95 | 95 | 95 | 95 | 100 | 95 | — | 40 | 90 |
| Groundsel | 100 | 100 | — | 100 | 100 | 100 | 100 | 100 | 100 | 100 | 100 | — | — | — | | 95 | — | 100 | 95 | — | — | | | 100 | — | — | 100 |
| Guineagrass | 100 | 100 | 100 | 95 | 80 | 80 | 100 | 100 | 100 | 100 | 100 | 100 | 100 | 100 | 100 | 95 | 100 | 100 | 100 | 95 | 100 | 100 | 100 | 100 | 95 | 60 | 90 |
| Horseweed | 100 | 100 | — | — | — | 100 | — | 100 | 100 | 100 | 100 | — | — | — | | 100 | 100 | 100 | 85 | 100 | 100 | | | 100 | 100 | — | 100 |
| Itchgrass | 95 | 95 | 70 | 95 | 85 | 75 | 80 | 100 | 100 | 100 | 95 | 95 | 100 | 100 | 100 | 80 | 85 | 95 | 90 | 60 | 80 | 100 | 95 | 90 | — | 40 | 60 |
| Johnsongrass | 100 | 100 | 70 | 95 | 80 | 60 | 85 | 100 | 100 | 95 | 100 | 80 | 85 | 85 | 100 | 80 | 85 | 100 | 95 | 80 | 90 | 70 | 85 | 100 | 90 | 20 | 80 |
| Kochia | 100 | 100 | 85 | 100 | 100 | 90 | 95 | 100 | 95 | 100 | 100 | — | — | — | | | | | | | | | | 100 | 100 | 80 | 75 |
| Mallow | 80 | 100 | 70 | 95 | 95 | 95 | 90 | 100 | 100 | 95 | 95 | 90 | 85 | 80 | 100 | 90 | 95 | 100 | 95 | 95 | 60 | 80 | — | 50 | — | 60 | 50 |
| Morningglory | 80 | 100 | 10 | 70 | 40 | 100 | 40 | 100 | 100 | 70 | 95 | 60 | 90 | 70 | | 60 | 90 | 80 | 75 | 95 | 75 | 50 | 40 | 60 | — | 10 | 50 |
| Nutsedge, Purple | 95 | 100 | 100 | 75 | 80 | 50 | 60 | 95 | — | 80 | 80 | 85 | 60 | 90 | | 100 | — | 75 | — | 100 | 80 | 50 | 80 | — | 100 | 30 | 60 |
| Prickly Sida | — | — | 20 | 100 | 95 | 95 | 95 | — | — | 95 | 100 | 100 | 100 | 100 | 100 | 95 | 95 | 100 | 100 | 100 | 95 | 100 | 100 | — | — | 0 | — |
| Purslane | 100 | 100 | 20 | 100 | 95 | 100 | 100 | 100 | 100 | 100 | 100 | — | — | — | | 100 | — | 100 | 100 | — | — | | | 100 | 100 | 0 | 100 |
| Quackgrass | 80 | 100 | 50 | 95 | 90 | 80 | 60 | 100 | 90 | 95 | 100 | 95 | 95 | 95 | 100 | 95 | 85 | 95 | 95 | 60 | 90 | 95 | 95 | 70 | — | 30 | 60 |
| Ragweed | 85 | 95 | 50 | 95 | 95 | 40 | 80 | 100 | 95 | 95 | 95 | 100 | 100 | 100 | 100 | 70 | 40 | 100 | 100 | 80 | 60 | 100 | 100 | 70 | — | 20 | 80 |
| Ryegrass, Italian | 95 | 100 | 40 | 95 | 95 | 95 | 95 | 100 | 95 | 100 | 100 | 95 | 100 | 100 | 100 | 95 | 95 | 95 | 80 | 85 | 95 | 95 | 100 | 95 | — | 40 | 60 |
| Sandbur | 100 | 100 | 40 | 80 | 85 | 60 | 80 | 80 | 80 | 60 | 95 | 100 | 100 | 100 | 100 | 70 | 100 | 80 | 70 | 60 | 80 | 100 | 95 | 80 | — | 30 | 20 |
| Signalgrass | 90 | 95 | 40 | 95 | 70 | 50 | 80 | 95 | 100 | 95 | 95 | 80 | 100 | 80 | 100 | 60 | 80 | 90 | 85 | 85 | 90 | 95 | 80 | 75 | — | 30 | 70 |
| Sowthistle | 95 | 100 | 95 | 95 | 95 | 95 | 95 | 100 | 95 | 95 | 95 | 100 | 100 | 100 | 95 | 95 | — | 100 | 95 | — | — | 95 | 100 | 95 | 100 | 95 | 95 |
| Spiderwort | 100 | 95 | 60 | 90 | 70 | 100 | 80 | 80 | 75 | 95 | 95 | 90 | 90 | 95 | 100 | 80 | 95 | 95 | 100 | 100 | 90 | 80 | 90 | 70 | 80 | 20 | 50 |
| Spurge, Spotted | — | — | — | 100 | 100 | — | 100 | — | — | — | — | — | — | — | | | | | | | | | | — | — | — | — |
| Surinam Grass | 90 | 100 | 60 | 100 | 90 | 70 | 80 | 95 | 100 | 100 | 100 | 100 | 100 | 100 | 100 | 75 | 95 | 100 | 95 | 70 | 90 | 100 | 100 | 80 | — | 30 | 90 |
| Thistle | 80 | 80 | 10 | 100 | 80 | — | 100 | 100 | 70 | — | — | 100 | 100 | 100 | | 80 | — | 100 | 100 | — | 60 | 95 | 100 | 60 | 70 | 10 | — |
| | 250 g ai/ha | | | | | | | | | | | | | | | | | | | | 125 g ai/ha | | | | | | |
| | Compounds | | | | | | | | | | | | | | | | | | | | Compounds | | | | | | |
| | 14 | 16 | 144 | 152 | 156 | 162 | 193 | 222 | 268 | 293 | 294 | 299 | 372 | 384 | 385 | 386 | 391 | 392 | 393 | 395 | 2 | 6 | 7 | 13 | 14 | 16 | 144 |
| Barnyardgrass | 85 | 80 | 80 | 85 | 100 | 100 | 85 | 100 | 100 | 100 | 95 | 95 | 80 | 85 | 70 | 100 | 100 | 90 | 80 | 95 | 80 | 95 | 30 | 80 | 80 | 80 | 60 |
| Beggarticks, Hairy | — | — | — | — | — | — | — | — | 100 | 100 | 100 | 100 | | | | | | | | | — | — | 10 | — | — | — | — |
| Beggarticks, Bip. | | | | | | | | | | | | | 50 | 100 | 30 | 40 | 100 | 80 | 100 | 100 | | | | | | | |
| Bermudagrass | 95 | 90 | 85 | 95 | 100 | 90 | 100 | 100 | 95 | 95 | 95 | 100 | 60 | 95 | 60 | 95 | 95 | 85 | 95 | 95 | 95 | 95 | 50 | 95 | 95 | 90 | 80 |
| Bindweed, Field | 75 | 50 | 50 | 60 | 100 | 100 | 80 | 95 | 40 | 60 | 90 | 95 | 50 | 80 | 20 | 60 | 90 | 100 | 60 | 60 | — | 60 | 10 | 30 | 75 | 50 | — |
| Black Mustard | 100 | 85 | 75 | 95 | 100 | 80 | 100 | 100 | 100 | 100 | 100 | 100 | 100 | 85 | 30 | 70 | 100 | 85 | 95 | 90 | 100 | 100 | 30 | 100 | 75 | 85 | 70 |
| Bluegrass | 95 | 95 | 100 | 95 | 100 | 100 | 100 | 100 | 95 | 95 | 95 | 95 | 95 | 80 | 30 | 95 | 95 | 80 | 75 | 95 | 100 | 100 | 60 | 60 | 95 | 95 | 90 |
| Bromegrass, Downy | 80 | 85 | 60 | 60 | 100 | 90 | 80 | 90 | 80 | 80 | 100 | 100 | 80 | 40 | 20 | 40 | 95 | 30 | 60 | 95 | 90 | 95 | 10 | 30 | 30 | 70 | 50 |
| Chickweed | 95 | 95 | 95 | 100 | 100 | 95 | 100 | 100 | — | — | — | — | 95 | 95 | 60 | 95 | 100 | 95 | 100 | 100 | 90 | 95 | — | 90 | 95 | 95 | 90 |
| Crabgrass, Large | 100 | 90 | 50 | 100 | 95 | 95 | 100 | 100 | 95 | 95 | 95 | 100 | 70 | 100 | 80 | 95 | 95 | 95 | 95 | 100 | 80 | 90 | 60 | 80 | 95 | 50 | 40 |
| Dallisgrass | 95 | 90 | 60 | 85 | 100 | 95 | 95 | 100 | 85 | 95 | 80 | 100 | 80 | 75 | 50 | 75 | 95 | 75 | 75 | 90 | 90 | 95 | 40 | 75 | 85 | 80 | 30 |
| Foxtail, Green | 100 | 80 | 80 | 85 | 100 | 100 | 80 | 100 | 100 | 95 | 100 | 100 | 60 | 40 | 60 | 95 | 95 | 70 | 70 | 80 | 80 | 95 | 10 | 30 | 75 | 60 | 50 |
| Goosegrass | 90 | 85 | 80 | 80 | 100 | 95 | 90 | 90 | 95 | 90 | 95 | 100 | 50 | 85 | 50 | 95 | 90 | 85 | 95 | 90 | 90 | 95 | 40 | 80 | 90 | 80 | 50 |
| Groundsel | 100 | 100 | 100 | 100 | 100 | 80 | 100 | 100 | — | — | — | — | 70 | — | 60 | 50 | 100 | 60 | — | — | 70 | 100 | — | 100 | 80 | 20 | 100 |
| Guineagrass | 95 | 80 | 60 | 80 | 100 | 100 | 100 | 100 | 100 | 100 | 100 | 100 | 80 | 95 | 100 | 100 | 100 | 95 | 90 | 100 | 80 | 95 | 40 | 60 | 90 | 60 | 20 |
| Horseweed | — | — | 100 | — | 100 | 100 | 100 | 100 | — | — | — | — | 95 | 95 | 0 | 95 | 100 | 75 | 100 | 100 | 100 | 100 | — | 100 | — | — | 100 |
| Itchgrass | 85 | 75 | 70 | 75 | 100 | 95 | 80 | 95 | 75 | 90 | 75 | 90 | 60 | 70 | 20 | 70 | 90 | 85 | 30 | 60 | 70 | 95 | 30 | 50 | 70 | 50 | 70 |
| Johnsongrass | 95 | 75 | 60 | 80 | 100 | 100 | 85 | 90 | 50 | 60 | 75 | 80 | 70 | 80 | 30 | 80 | 85 | 90 | 60 | 90 | 90 | 90 | 10 | 50 | 70 | 70 | 50 |
| Kochia | 95 | 70 | 90 | 85 | 100 | 95 | 100 | 100 | — | — | — | — | | | | | | | | | 100 | 100 | 0 | 70 | 95 | 70 | 60 |
| Mallow | 95 | 85 | 70 | 90 | 100 | 100 | 95 | 95 | 80 | 50 | 40 | 95 | 80 | 70 | 20 | 70 | 100 | 95 | 95 | — | — | 100 | 60 | 50 | 85 | 60 | 60 |
| Morningglory | 60 | 30 | 20 | 40 | 100 | 85 | 50 | 40 | 40 | 40 | 10 | 90 | 50 | 80 | 10 | 40 | 80 | 40 | 95 | 75 | 50 | 70 | 10 | 50 | 30 | 20 | 20 |
| Nutsedge, Purple | 60 | 70 | 30 | 50 | — | — | 50 | 70 | 85 | 40 | 80 | 80 | 50 | — | — | — | 75 | — | 20 | 70 | — | 100 | 0 | 20 | 50 | 70 | 30 |
| Prickly Sida | 85 | 90 | 90 | 60 | — | — | 70 | 100 | 100 | 80 | 100 | 100 | 95 | 60 | 100 | 100 | 100 | 75 | 75 | 95 | — | — | 0 | — | 75 | 90 | 90 |
| Purslane | 100 | 95 | 90 | 50 | 100 | 100 | 100 | 100 | — | — | — | — | 50 | — | 100 | 100 | 100 | 100 | — | — | 100 | 100 | 0 | 60 | 100 | 95 | 20 |
| Quackgrass | 90 | 80 | 70 | 50 | 95 | 75 | 70 | 100 | 95 | 80 | 80 | 95 | 95 | 85 | 30 | 90 | 95 | 70 | 60 | 85 | 60 | 100 | 20 | 20 | 50 | 70 | 30 |
| Ragweed | 95 | 70 | 40 | 80 | 95 | 75 | 80 | 95 | 100 | 70 | 85 | 90 | 40 | 40 | 0 | 60 | 95 | 70 | 20 | 60 | 60 | 80 | 20 | 20 | 60 | 50 | 30 |
| Ryegrass, Italian | 85 | 95 | 80 | 80 | 100 | 95 | 100 | 100 | 95 | 95 | 100 | 100 | 95 | 80 | 60 | 95 | 95 | 70 | 85 | 90 | 80 | 95 | 40 | 40 | 70 | 90 | 60 |
| Sandbur | 40 | 80 | 30 | 70 | 75 | 80 | 20 | 70 | 100 | 100 | 80 | 100 | 60 | 70 | 40 | 40 | 80 | 20 | 50 | 80 | 70 | 100 | 20 | 0 | 40 | 70 | 30 |
| Signalgrass | 95 | 60 | 30 | 70 | 90 | 100 | 70 | 95 | 80 | 95 | 70 | 100 | 40 | 80 | 20 | 85 | 90 | 70 | 75 | 85 | 70 | 75 | 20 | 30 | 60 | 60 | 20 |
| Sowthistle | 95 | 90 | 90 | 95 | 100 | 80 | 95 | 90 | 100 | 95 | 95 | 100 | 40 | — | 60 | 100 | 100 | 95 | — | — | 95 | 95 | 60 | 90 | 90 | 60 | 50 |
| Spiderwort | 80 | 60 | 60 | 75 | 50 | 75 | 90 | — | 85 | 80 | 85 | 95 | 70 | 60 | 0 | 70 | 90 | 95 | 100 | 60 | 60 | 75 | 10 | 50 | 60 | 10 | 60 |
| Spurge, Spotted | 100 | 100 | — | 100 | — | — | — | — | — | — | — | — | | | | | | | | | — | — | — | — | 100 | 100 | — |
| Surinam Grass | 95 | 80 | 50 | 70 | 95 | 95 | 90 | 100 | 100 | 100 | 100 | 95 | 60 | 80 | 40 | 80 | 90 | 60 | 30 | 80 | 80 | 90 | — | 60 | 90 | 75 | 50 |
| Thistle | 60 | 50 | — | 40 | 100 | 60 | — | — | 100 | 80 | 100 | 100 | — | — | 95 | 40 | 100 | 50 | — | — | 60 | 70 | 10 | — | 60 | 50 | — |
| | 125 g ai/ha | | | | | | | | | | | | | | | | | 62 g ai/ha | | | | | | | | | |
| | Compounds | | | | | | | | | | | | | | | | | Compounds | | | | | | | | | |
| | 152 | 156 | 162 | 193 | 222 | 268 | 293 | 294 | 299 | 372 | 384 | 385 | 386 | 391 | 392 | 393 | 395 | 2 | 6 | 7 | 13 | 14 | 16 | 144 | 152 | 156 | 162 |
| Barnyardgrass | 75 | 100 | 100 | 75 | 90 | 85 | 60 | 95 | 85 | 70 | 80 | 50 | 80 | 100 | 75 | 60 | 60 | 60 | 90 | 0 | 30 | 60 | 70 | 30 | 60 | 100 | 60 |
| Beggarticks, Hairy | — | — | — | — | — | 95 | 100 | 100 | 100 | | | | | | | | | — | — | 0 | — | — | — | — | — | — | — |
| Beggarticks, Bip. | | | | | | | | | | 40 | — | 30 | — | 80 | 10 | 100 | 100 | | | | | | | | | | |
| Bermudagrass | 90 | 100 | 80 | 100 | 100 | 90 | 90 | 90 | 100 | 60 | 95 | 60 | 80 | 90 | 60 | 90 | 95 | 90 | 90 | 10 | 80 | 90 | 80 | 50 | 70 | 95 | 70 |
| Bindweed, Field | 20 | 90 | 80 | 50 | 40 | 20 | 40 | 10 | 60 | 10 | — | 10 | 20 | 90 | 30 | 20 | 40 | 10 | 30 | 0 | 30 | 60 | 0 | 0 | 10 | 60 | 30 |
| Black Mustard | 90 | 100 | 50 | 100 | 100 | 100 | 95 | 100 | 100 | 100 | 80 | — | — | 80 | — | 80 | 60 | 60 | 100 | 10 | 70 | 60 | 80 | 20 | 90 | 100 | 20 |
| Bluegrass | 80 | 95 | 100 | 100 | 100 | 95 | 95 | 95 | 95 | 80 | 75 | 10 | 60 | 95 | 80 | 30 | 80 | 95 | 100 | 0 | 50 | 50 | 90 | 80 | 60 | 95 | 100 |
| Bromegrass, Downy | 30 | 80 | 80 | 30 | 90 | 80 | — | 80 | — | 70 | 40 | 10 | 10 | 30 | 20 | 10 | 40 | 50 | 90 | 0 | 0 | 20 | 10 | 20 | 0 | 60 | 40 |
| Chickweed | 95 | 100 | 95 | 100 | 100 | — | — | — | — | 90 | 95 | 20 | 95 | 90 | 95 | 60 | 90 | 90 | 90 | — | 90 | 90 | 70 | 50 | 90 | 80 | — |
| Crabgrass, Large | 70 | 90 | 90 | 80 | 100 | 80 | 95 | 75 | 100 | 50 | 100 | 80 | 95 | 95 | 95 | 80 | 100 | 70 | 80 | 20 | 60 | 95 | 20 | 0 | 40 | — | 50 |
| Dallisgrass | 80 | 95 | 95 | 95 | 80 | 80 | 60 | 75 | 85 | 50 | 70 | 30 | 70 | 80 | 60 | 10 | 70 | 60 | 90 | 40 | 50 | 40 | 50 | 0 | 50 | 90 | 70 |
| Foxtail, Green | 70 | 100 | 85 | 30 | 50 | 95 | 50 | 80 | 100 | 50 | — | 30 | 60 | 80 | — | 20 | 40 | 40 | 95 | 0 | 10 | 30 | 60 | 10 | 20 | 100 | 30 |
| Goosegrass | 70 | 95 | 90 | 80 | 75 | 80 | 90 | 95 | 100 | 40 | 80 | 20 | 80 | 80 | 70 | 50 | 80 | 80 | 85 | 0 | 50 | 70 | 60 | 20 | 30 | 95 | 60 |
| Groundsel | 100 | 100 | 40 | 100 | 100 | — | — | — | — | 70 | — | 0 | 30 | 95 | 60 | — | — | 60 | 100 | — | 70 | 80 | — | 10 | 95 | 100 | 10 |
| Guineagrass | 50 | 100 | 100 | 70 | 95 | 85 | 100 | 90 | 85 | 80 | 95 | 60 | 80 | 80 | 80 | 70 | 100 | 70 | 95 | 0 | 20 | 60 | 20 | 10 | 20 | 90 | 50 |
| Horseweed | — | 100 | 100 | 100 | 100 | — | — | — | — | 80 | 75 | — | 75 | 95 | 60 | 95 | 100 | 80 | 100 | — | 80 | — | — | 70 | — | 95 | 100 |
| Itchgrass | 60 | 100 | 95 | 70 | 95 | 60 | 10 | — | 75 | 60 | 60 | 20 | 70 | 80 | 85 | 10 | 60 | 60 | 90 | 0 | 20 | 10 | 50 | 20 | 20 | 100 | 40 |
| Johnsongrass | 50 | 100 | 100 | 75 | 80 | 50 | 10 | 30 | 60 | 50 | 75 | 10 | 75 | 60 | 30 | 60 | 80 | 90 | 90 | 0 | 0 | 30 | 40 | 30 | 30 | 95 | 60 |
| Kochia | 85 | 100 | 70 | 100 | 100 | — | — | — | — | | | | | | | | | 90 | 100 | 0 | 30 | 10 | 60 | 0 | 0 | 100 | 60 |
| Mallow | 80 | 100 | 70 | 95 | 80 | 50 | 40 | 40 | 75 | 50 | 70 | 10 | 60 | 80 | 80 | 60 | 50 | 30 | 70 | 0 | 50 | 60 | 60 | 60 | 40 | 70 | 50 |
| Morningglory | 10 | 100 | 60 | 20 | 30 | 10 | 10 | 10 | 75 | 20 | 60 | 10 | 20 | 60 | 40 | 60 | 40 | 30 | 20 | 0 | 0 | 10 | 0 | 0 | 10 | 30 | 30 |
| Nutsedge, Purple | 20 | — | — | 30 | 40 | 70 | — | 80 | — | — | — | — | — | — | — | — | 50 | — | 60 | 0 | 0 | 20 | 60 | 0 | 0 | — | 0 |
| Prickly Sida | 20 | — | — | 70 | 70 | 80 | — | 100 | 85 | — | — | 100 | 100 | 100 | — | 70 | 95 | — | — | 0 | — | 30 | 40 | 10 | 20 | — | — |
| Purslane | 0 | 100 | 100 | 60 | 100 | — | — | — | — | 40 | — | 100 | 100 | 95 | 90 | — | — | — | 100 | 0 | 40 | 100 | 0 | 0 | 0 | 100 | 100 |
| Quackgrass | 20 | 90 | 60 | 60 | 80 | 85 | 70 | 80 | 80 | 60 | 70 | 20 | 80 | 90 | 60 | 50 | 80 | 30 | 95 | 20 | 0 | 30 | 40 | 10 | 10 | 50 | 20 |
| Ragweed | 60 | 90 | 60 | 50 | 85 | 60 | 50 | 75 | 85 | 20 | 10 | 0 | 20 | 75 | 30 | 10 | 20 | 20 | 50 | 0 | 10 | 30 | 20 | 0 | 20 | 50 | 20 |
| Ryegrass, Italian | 50 | 95 | 95 | 90 | 100 | 95 | 60 | 80 | 95 | 70 | 75 | 30 | 60 | 80 | 60 | 20 | 80 | 60 | 90 | 0 | 10 | 30 | 60 | 10 | 20 | 60 | 50 |
| Sandbur | 30 | 60 | 70 | 10 | 40 | 70 | 10 | 70 | 60 | 30 | 60 | 10 | 40 | 70 | 10 | 50 | 60 | 60 | 85 | 0 | 0 | 10 | 10 | 10 | 10 | 50 | 20 |
| Signalgrass | 40 | — | 70 | 60 | 60 | 60 | 50 | 50 | 80 | 10 | 70 | 20 | 30 | 70 | 30 | 30 | 75 | 50 | 75 | 0 | 10 | 20 | 10 | 10 | 10 | 85 | — |
| Sowthistle | 80 | 100 | 60 | 95 | 90 | 100 | 75 | 80 | 100 | 40 | — | 0 | 60 | 75 | 95 | — | — | 90 | 95 | 0 | 80 | 30 | 60 | 10 | 80 | 60 | 40 |
| Spiderwort | 20 | 50 | 40 | 80 | — | 70 | 60 | — | 85 | 70 | 30 | 0 | 10 | 90 | 50 | 100 | — | 20 | 70 | 0 | 10 | 60 | 10 | 60 | 10 | 30 | 20 |
| Spurge, Spotted | 100 | — | — | — | — | — | — | — | — | | | | | | | | | — | — | — | — | 90 | 100 | — | — | — | — |
| Surinam Grass | 50 | 75 | 75 | 80 | 90 | 90 | 80 | 95 | 95 | 40 | 60 | 20 | 80 | 70 | 60 | 20 | 75 | 60 | 85 | — | 20 | 40 | 60 | 10 | 20 | 50 | 60 |
| Thistle | 20 | 100 | 50 | — | — | 60 | 60 | 50 | 100 | 40 | — | 50 | — | 100 | 10 | — | — | 50 | 50 | 0 | — | 10 | 10 | — | 20 | 80 | 50 |

| | 62 g ai/ha | | | | | | | | | | | 31 g ai/ha |
|---|---|---|---|---|---|---|---|---|---|---|---|---|
| | Compounds | | | | | | | | | | | Compound |
| | 193 | 222 | 268 | 299 | 372 | 384 | 385 | 391 | 392 | 393 | 395 | 13 |
| Barnyardgrass | 20 | 50 | 60 | 60 | 50 | 40 | 20 | 50 | 75 | 0 | 50 | 0 |
| Beggarticks, Hairy | — | — | 40 | 50 | | | | | | | | |
| Beggarticks, Bip. | | | | | 10 | 10 | 0 | 40 | — | 30 | 40 | |
| Bermudagrass | 85 | 80 | 60 | 80 | 10 | 80 | 20 | 85 | 60 | 40 | 90 | 40 |
| Bindweed, Field | 50 | — | 0 | 10 | 10 | 80 | 10 | 20 | 30 | 0 | 10 | 0 |
| Black Mustard | 80 | 90 | 90 | 100 | 100 | 60 | 0 | 50 | 60 | 10 | 40 | 30 |
| Bluegrass | 80 | 90 | 80 | 85 | 30 | 50 | 0 | 95 | 10 | 10 | 30 | 0 |
| Bromegrass, Downy | 10 | 30 | — | — | 20 | 20 | 10 | 10 | 10 | 0 | 40 | 0 |
| Chickweed | 80 | 80 | — | — | 60 | 75 | 20 | 90 | 60 | — | — | 40 |
| Crabgrass, Large | 60 | 70 | 70 | 90 | 10 | 90 | 80 | 60 | 75 | 10 | 80 | 0 |
| Dallisgrass | 40 | 50 | 70 | 75 | 10 | 10 | 10 | 60 | 10 | 0 | 50 | 0 |
| Foxtail, Green | 10 | 10 | 70 | 50 | 10 | 10 | 10 | 60 | 10 | 10 | 10 | 0 |
| Goosegrass | 30 | 60 | 70 | 100 | 40 | 60 | 20 | 30 | 20 | 10 | 80 | 10 |
| Groundsel | 20 | 30 | — | — | 20 | — | 0 | — | 10 | — | — | |
| Guineagrass | 60 | 90 | 70 | 70 | 40 | 95 | — | 60 | 75 | 10 | 80 | 0 |
| Horseweed | 80 | 100 | — | — | 80 | — | 0 | 80 | 60 | 90 | 90 | |
| Itchgrass | 20 | 75 | 50 | 60 | 30 | 40 | 20 | 50 | 50 | 0 | 20 | 0 |
| Johnsongrass | 20 | 70 | 30 | 40 | 30 | 40 | 10 | 40 | 10 | 30 | 30 | 0 |
| Kochia | 100 | 30 | — | — | | | | | | | | 10 |
| Mallow | 60 | 70 | 50 | 50 | 50 | 50 | 10 | 80 | 10 | 10 | 20 | 40 |
| Morningglory | 0 | — | 10 | 10 | 10 | 10 | 10 | 10 | 10 | 0 | 20 | 0 |
| Nutsedge, Purple | 0 | 10 | 50 | 50 | 30 | 60 | — | — | — | — | — | 0 |
| Prickly Sida | 20 | 20 | 60 | 85 | 20 | 60 | 10 | 50 | — | 30 | 60 | |
| Purslane | — | 70 | — | — | 10 | — | 100 | 50 | — | — | — | 40 |
| Quackgrass | 10 | 10 | 40 | 80 | 30 | 50 | 10 | 40 | 10 | 50 | 60 | 0 |
| Ragweed | 50 | 30 | 50 | 20 | 10 | 0 | 0 | 50 | 30 | 0 | 10 | 0 |
| Ryegrass, Italian | 20 | 50 | 95 | 70 | 60 | 20 | 10 | — | 10 | 10 | 20 | 0 |
| Sandbur | 0 | 0 | 10 | 10 | 20 | 10 | 0 | 10 | 0 | 0 | 50 | 0 |
| Signalgrass | 10 | 10 | 20 | 60 | 0 | 10 | 10 | 40 | 10 | 0 | 10 | 10 |
| Sowthistle | 80 | 75 | 80 | 100 | 10 | — | — | 50 | 20 | — | — | 30 |
| Spiderwort | 10 | 60 | 50 | 75 | 10 | — | — | 70 | 10 | 0 | 10 | 0 |
| Spurge, Spotted | — | — | — | — | | | | | | | | |
| Surinam Grass | 10 | 50 | 60 | 50 | 20 | 30 | 10 | 20 | 20 | 0 | 10 | 0 |
| Thistle | — | — | 60 | 100 | 0 | 10 | 0 | 50 | — | — | — | |

Test F

Seeds of plant species selected from annual blugrass (Poa annua L.), blackgrass (Alopecurus myosuroides Huds.), catchweed bedstraw (Gallium aparine L.), common chickweed (Stellaria media (L.) Vill./Cyr.), downy bromegrass (Bromus tectorum L.), green foxtail (Setaria viridis (L.) Beauv.), Italian ryegrass (Lolium multiflorum Lam.), kochia (Kochia scoparia (L.). Schrad.), lambsquarters (Chenopodium album L.), littleseed canarygrass (Phalaris minor Retz.), pigweed (Amaranthus retroflexus L.), Russian thistle (Salsola kali L. ssp. Ruthenica (Iljin) Soo), wild buckwheat (Polygonum convolvulus L.), wild mustard (Sinapis arvensis L.), wild oat (Avena fatua L.), windgrass (Apera spica-venti (L.) Beauv.), winter barley (Hordeum vulgare L.), and wheat (Triticum aestivum L.) were planted and treated preemergence with test chemicals formulated in a non-phytotoxic solvent mixture which included a surfactant.

At the same time, plants selected from these crop and weed species were treated with postemergence applications of some of the test chemicals formulated in the same manner. Plants ranged in height from 2 to 18 cm (1- to 4-leaf stage) for postemergence treatments. Treated plants and controls were maintained in a greenhouse for 12 to 14 days, after which time all species were compared to controls and visually evaluated. Plant response ratings, summarized in Table F, are based on a scale of 0 to 100 where 0 is no effect and 100 is complete control. A dash (-) response means no test result.

TABLE F
Postemergence

| | 500 g ai/ha | 250 g ai/ha | | | 125 g ai/ha | | | | 62 g ai/ha | | | 31 g ai/ha |
|---|---|---|---|---|---|---|---|---|---|---|---|---|
| | Compound | Compounds | | | Compounds | | | | Compounds | | | Compounds |
| | 162 | 9 | 162 | 248 | 9 | 162 | 223 | 248 | 9 | 162 | 248 | 248 |
| Barley, Winter | 80 | 75 | 80 | 40 | 30 | 50 | 0 | 30 | 20 | 40 | 30 | 25 |
| Blackgrass | 100 | 85 | 100 | 60 | 70 | 80 | 60 | 60 | 40 | 70 | 50 | 45 |
| Bluegrass | 100 | 80 | 90 | 70 | 70 | 60 | 50 | 60 | 50 | 60 | 50 | 50 |
| Bromegrass, Downy | 90 | 65 | 80 | 40 | 60 | 50 | 20 | 40 | 30 | 50 | 40 | 40 |
| Buckwheat, Wild | | 60 | — | 60 | 60 | — | 40 | 50 | 60 | — | 50 | 50 |
| Canarygrass | 100 | 65 | 100 | 45 | 40 | 70 | 40 | 40 | 0 | 70 | 40 | 35 |
| Chickweed | 100 | 60 | 100 | 100 | 60 | 80 | 60 | 98 | 30 | 70 | 65 | 65 |
| Foxtail, Green | 100 | 50 | 60 | 65 | 40 | 50 | 60 | 60 | 40 | 50 | 60 | 50 |
| Galium | | 65 | — | 60 | 45 | — | 60 | 60 | 40 | — | 50 | 50 |
| Kochia | 100 | 90 | 100 | 65 | 65 | 100 | 75 | 65 | 65 | 100 | 60 | 60 |
| Lambsquarters | 100 | 90 | 100 | 60 | 90 | 100 | 65 | 60 | 80 | 100 | 60 | 35 |
| Mustard, Wild | 100 | 90 | 100 | 60 | 90 | 80 | 65 | 60 | 65 | 60 | 40 | 30 |
| Oat, Wild | 100 | 65 | 100 | 65 | 45 | 80 | 35 | 60 | 35 | 70 | 45 | 45 |
| Pigweed | 100 | 95 | 100 | 70 | 90 | 90 | 65 | 70 | 65 | 70 | 65 | 65 |
| Ryegrass, Italian | 100 | 80 | 90 | 65 | 65 | 80 | 45 | 60 | 60 | 70 | 55 | 45 |
| Thistle | | 65 | — | 40 | 60 | — | 60 | 30 | 30 | — | 30 | 25 |
| Wheat | 70 | 65 | 50 | 20 | 0 | 40 | 0 | 5 | 0 | 20 | 5 | 0 |
| Windgrass | | 80 | — | 50 | 50 | — | 50 | 50 | 30 | — | 50 | 50 |

Preemergence

| | 500 g ai/ha | 250 g ai/ha | | | 125 g ai/ha | | | | 62 g ai/ha | | | 31 g ai/ha |
|---|---|---|---|---|---|---|---|---|---|---|---|---|
| | Compound | Compounds | | | Compounds | | | | Compounds | | | Compounds |
| | 162 | 9 | 162 | 248 | 9 | 162 | 223 | 248 | 9 | 162 | 248 | 248 |
| Barley, Winter | 100 | 75 | 100 | 35 | 65 | 100 | 25 | 0 | 0 | 70 | 0 | 0 |
| Blackgrass | 100 | 100 | 100 | 65 | 100 | 100 | 70 | 65 | 70 | 90 | 50 | 50 |
| Bluegrass | 100 | 100 | 100 | 100 | 100 | 100 | 100 | 100 | 85 | 100 | 70 | 70 |
| Bromegrass, Downy | 100 | 75 | 100 | 65 | 70 | 100 | 50 | 50 | 60 | 70 | 45 | 35 |
| Buckwheat, Wild | | 100 | — | 60 | 100 | — | 55 | 45 | 60 | — | 40 | 30 |
| Canarygrass | 100 | 75 | 100 | 75 | 60 | 100 | 50 | — | 50 | 100 | 45 | 40 |
| Chickweed | 100 | 100 | 100 | 100 | 100 | 100 | 100 | 100 | 100 | 100 | 100 | 100 |
| Foxtail, Green | 100 | 100 | 100 | 100 | 100 | 100 | 55 | 100 | 70 | 70 | 65 | 65 |
| Galium | 100 | 30 | 100 | 60 | 30 | 70 | 50 | 50 | 30 | 60 | 50 | 30 |
| Kochia | 100 | 100 | 100 | 100 | 100 | 100 | 75 | 65 | 60 | 60 | 60 | 50 |
| Lambsquarters | 100 | 100 | 100 | 100 | 100 | 100 | 95 | 100 | 100 | 100 | 40 | 40 |
| Mustard, Wild | 100 | 100 | 100 | 50 | 100 | 80 | 100 | 50 | 100 | 80 | 50 | 40 |
| Oat, Wild | 100 | 75 | 100 | 65 | 65 | 100 | 70 | 50 | 45 | 100 | 45 | 40 |
| Pigweed | 100 | 100 | 100 | 95 | 100 | 100 | 100 | 95 | 100 | 90 | 75 | 70 |
| Ryegrass, Italian | 100 | 100 | 100 | 60 | 95 | 100 | 65 | 50 | 65 | 100 | 50 | 30 |
| Thistle | | 100 | — | 45 | 65 | — | 60 | 30 | 45 | — | 30 | 30 |
| Wheat | 100 | 65 | 100 | 10 | 60 | 100 | 0 | 0 | 0 | 60 | 0 | 0 |
| Windgrass | | 100 | — | 100 | 100 | — | 100 | 100 | 80 | — | 70 | 70 |

Test G

This test evaluated the safening of compounds of the invention on corn (maize; Zea mays L.) cv. ‘Pioneer 33G26’ by seed treatment with naphthalic anhydride (1,8-naphthalic anhydride). All corn seed had been first treated with fludioxonil and metaxyl applied at the manufacturer's recommended rate as per the Pioneer 33G26 label. Some of the corn seed were subsequently also treated with naphthalic anhydride as a 1% by weight seed dressing. The corn seed were planted in pots containing pasteurized Sassafras sandy loam soil, and then treatments were applied preemergence the same day. Treatments were applied by spraying the test compounds formulated in a non-phytotoxic solvent mixture, using a flat fan nozzle and a spray volume of 280 L/ha. The treatments were triply replicated and the results subsequently averaged. The pots were placed on a greenhouse bench using a complete randomized block design except for the first replicate, which was unrandomized. The plants were grown in the greenhouse and watered as needed with a dilute nutrient solution containing 200 ppm of N. Illumination was daylight supplemented by artificial sources to maintaining a photoperiod of 16 hours. The temperature was maintained at 28±2° C. during the day and 23±2° C. at night. The plant response was visually rated 25 days after treatment in comparison to untreated controls using a scale of 0 to 100, with 0 representing no effect and 100 representing complete plant death. The results are listed in Table G.

TABLE G
Results from using naphthalic anhydride to safen
compounds of the invention on corn

| | | Without | With 1% |
|---|---|---|---|
| | | naphthalic | naphthalic |
| | Application | anhydride seed | anhydride seed |
| Treatment | Rate (g a.i./ha) | treatment | treatment |
| Only spray solvent | 0 | 0 | 0 |
| Compound 2 | 62 | 65 | 50 |
| | 125 | 72 | 55 |
| | 250 | 78 | 68 |
| | 500 | 85 | 72 |
| Compound 6 | 62 | 65 | 60 |
| | 125 | 68 | 62 |
| | 250 | 88 | 78 |
| | 500 | 92 | 70 |
| Compound 156 | 62 | 2 | 0 |
| | 125 | 30 | 0 |
| | 250 | 65 | 17 |
| | 500 | 88 | 17 |
| Compound 162 | 62 | 15 | 0 |
| | 125 | 30 | 25 |
| | 250 | 68 | 60 |
| | 500 | 85 | 63 |

Test H

This test evaluated the safening of compounds of the invention on wheat (Triticum aestivumL.) cv. ‘Retical’ by seed treatment with naphthalic anhydride. Some of the wheat seed was treated with napthalic anhydride as a 1% by weight seed dressing. The wheat seed were planted in pots containing pasteurized Sassafras sandy loam soil. For postemergence testing the plants were grown 8 days to the 2-leaf stage at time of treatment. Preemergence treatments were applied the same day that the seeds were planted. Treatments were applied by spraying the test compounds formulated in a non-phytotoxic solvent mixture, using a flat fan nozzle and a spray volume of 280 L/ha. The preemergence treatments were triply replicated and the results subsequently averaged. The pots were placed on a growth chamber bench using a complete randomized block design for the preemergence test except for the first replicate, which was unrandomized. The plants were grown in the growth chamber and watered as needed with a dilute nutrient solution containing 200 ppm of N. Illumination was provided by fluorescent lamps giving 200-300 μE/m²/S of photosynthetically active radiation over a 14-hour photoperiod. The temperature was maintained at 23±2° C. during the day and 17±2° C. at night. The effects of the treatments were rated 25 days after preemergence treatment and 14 days after postemergence treatment. The plant response was visually rated in comparison to untreated controls using a scale of 0 to 100, with 0 representing no effect and 100 representing complete plant death. The results for the compounds tested preemergence are listed in Table H1, and the results for the compounds tested postemergence are listed in Table H2.

TABLE H1
Results from using naphthalic anhydride to safen
compounds of the invention applied preemergence to wheat

| | | Without | With 1% |
|---|---|---|---|
| | | naphthalic | naphthalic |
| | Application | anhydride seed | anhydride seed |
| Treatment | Rate (g a.i./ha) | treatment | treatment |
| Only spray solvent | 0 | 0 | 0 |
| Compound 2 | 62 | 62 | 50 |
| | 125 | 82 | 72 |
| Compound 6 | 62 | 99 | 77 |
| | 125 | 100 | 100 |
| Compound 156 | 62 | 67 | 7 |
| | 125 | 100 | 70 |
| | 250 | 100 | 85 |
| | 500 | 100 | 99 |
| Compound 162 | 62 | 60 | 47 |
| | 125 | 98 | 62 |
| | 250 | 98 | 96 |

TABLE H2
Results from using naphthalic anhydride to safen
compounds of the invention applied postemergence to wheat

| | | Without | With 1% |
|---|---|---|---|
| | | naphthalic | naphthalic |
| | Application | anhydride seed | anhydride seed |
| Treatment | Rate (g a.i./ha) | treatment | treatment |
| Only spray solvent | 0 | 0 | 0 |
| Compound 2 | 62 | 60 | 30 |
| | 125 | 75 | 65 |
| Compound 6 | 62 | 75 | 65 |
| | 125 | 75 | 75 |
| Compound 162 | 62 | 5 | 5 |
| | 125 | 70 | 30 |

Test I

This test evaluated the safening of Compound 6 on barley (Hordeum vulgare L.) cv. ‘Boone’ and wheat (Triticum aestivum L.) cv. ‘Recital’ by Harmony® Extra Herbicide, which comprises 50 wt % thifensulfuron-methyl and 25 wt % tribenuron-methyl. Barley and wheat seeds were planted in pots containing a pasteurized blend of Matapeake soil and sand. For postemergence testing the plants were grown 10 days so the barley seedlings were at the 2-leaf stage and the wheat seedlings were at the 2-3-leaf stage at time of treatment. Preemergence treatments were applied the day after the seeds were planted. Treatments were applied by spraying Compound 6 and/or Harmony® Express in a non-phytotoxic solvent mixture, using flat fan nozzle and a spray volume of 280 L/ha. The treatments were triply replicated and the results subsequently averaged. The pots were placed on a greenhouse bench using a complete randomized block design except for the first replicate, which was unrandomized. The plants were grown in the greenhouse and watered as needed with a dilute nutrient solution containing 200 ppm of N. Illumination was daylight supplemented by artificial sources to maintain a photoperiod of 14 hours. The temperature was maintained at 23±2° C. during the day and 17±2° C. at night. The effects of the treatments were rated 25 days after preemergence treatment and 15 days after postemergence treatment. The plant response was visually rated in comparison to untreated controls using a scale of 0 to 100, with 0 representing no effect and 100 representing complete plant death.

Colby's Equation was used to calculate the expected additive herbicidal effect of the mixtures of Compound 6 with Harmony® Extra (i.e. a 2:1 mixture by weight of thifensulfuron-methyl and tribenuron-methyl). Colby's Equation (Colby, S. R. “Calculating Synergistic and Antagonistic Responses of Herbicide Combinations,” Weeds, 15(1), pp 20-22 (1967)) calculates the expected additive effect of herbicidal mixtures, and for two active ingredients is of the form:
P_{a+b}=Pₐ+P_{b}−(PₐP_{b}/100)
wherein
P_{a+b} is the percentage effect of the mixture expected from additive contribution of the individual components,
Pₐ is the observed percentage effect of the first active ingredient at the same use rate as in the mixture, and
P_{b} is the observed percentage effect of the second active ingredient at the same use rate as in the mixture.

The results and additive effects expected from Colby's Equation for the preemergence test are listed in Table I1, and the results and additive effects expected from Colby's Equation for the postemergence test are listed in Table I2.

TABLE I1
Results from using Harmony ® Extra Herbicide to safen
Compound 6 applied preemergence to barley and wheat

| Compound 6 | Thifensulfuronmethyl | Tribenuronmethyl | Barley | | Wheat | |
|---|---|---|---|---|---|---|
| (g a.i./ha) | (g a.i./ha) | (g a.i./ha) | Observed | Expected* | Observed | Expected* |
| 0 | 5.3 | 2.7 | 0 | — | 0 | — |
| 0 | 10.7 | 5.3 | 5 | — | 5 | — |
| 16 | 0 | 0 | 35 | — | 45 | — |
| 16 | 5.3 | 2.7 | 35 | 35 | 13 | 45 |
| 16 | 10.7 | 5.3 | 13 | 38 | 7 | 48 |
| 31 | 0 | 0 | 68 | — | 67 | — |
| 31 | 5.3 | 2.7 | 65 | 68 | 55 | 67 |
| 31 | 10.7 | 5.3 | 48 | 70 | 40 | 68 |

*Effects expected from Colby's Equation.

TABLE I2
Results from using Harmony ® Extra Herbicide to safen
Compound 6 applied postemergence to barley and wheat

| Compound 6 | Thifensulfuronmethyl | Tribenuronmethyl | Barley | | Wheat | |
|---|---|---|---|---|---|---|
| (g a.i./ha) | (g a.i./ha) | (g a.i./ha) | Observed | Expected* | Observed | Expected* |
| 0 | 5.3 | 2.7 | 0 | — | 0 | — |
| 0 | 10.7 | 5.3 | 7 | — | 8 | — |
| 16 | 0 | 0 | 47 | — | 58 | — |
| 16 | 5.3 | 2.7 | 40 | 47 | 45 | 58 |
| 16 | 10.7 | 5.3 | 33 | 50 | 43 | 62 |
| 31 | 0 | 0 | 73 | — | 65 | — |
| 31 | 5.3 | 2.7 | 68 | 73 | 58 | 65 |
| 31 | 10.7 | 5.3 | 63 | 75 | 43 | 68 |

*Effects expected from Colby's Equation.

Test J

Seeds of test plants consisting of barnyardgrass (ECHCG; Echinochloa crus-galli (L.) Beauv.), blackgrass (ALOMY; Alopecurus myosuroides Huds.), Surinam grass (BRADC; Urochloa decumbens (Staph) R. D. Webster, previously named Brachiaria decumbens Stapf), cocklebur (XANST, Xanthium strumarium L.), corn (ZEAMD, Zea mays L. cv. ‘Pioneer 33G26’), large crabgrass (DIGSA, Digitaria sanguinalis (L.) Scop.), giant foxtail (SETFA, Setaria faberi Herrm.), lambsquarters (CHEAL, Chenopodium album L.), morningglory (IPOCO, Ipomoea coccinea L.), pigweed (AMARE, Amaranthus retroflexus L.), velvetleaf (ABUTH, Abutilon theophrasti Medik.) wheat (TRZAS, Triticum aestivum L. cv. ‘Recital’) and wild oat (AVEFA, Avena fatua L.) were planted in Redi-Earth® planting medium (Scotts Company, 14111 Scottslawn Road, Marysville, Ohio 43041) comprising spaghnum peat moss and vermiculite. Seeds of small-seeded species were planted about 1 cm deep; larger seeds were planted about 2.5 cm deep. Plants were grown in a greenhouse using supplemental lighting to maintain a photoperiod of about 14 hours; daytime and nighttime temperatures were about 24-30° C. and 22-25° C., respectively. Balanced fertilizer was applied through the watering system. The plants were grown for 7 to 11 days so that at time of treatment the plants ranged in height from 2 to 18 cm (1- to 4-leaf stage). Treatments consisted of Compounds 2 and 6 (technical material), atrazine (90DF), terbacil (Sinbar® 80DF), hexazinone (Velpar® 75WG), diuron (Karmex® 80WP) and paraquat (Gramoxone® Extra, 37%) alone and in combination, suspended or dissolved in an aqueous solvent comprising a nonionic surfactant and applied as a foliage spray using a volume of 541 L/ha. Each treatment was triply replicated. The application solvent was observed to have no effect compared to untreated check plants. Treated plants and controls were maintained in the greenhouse and watered as needed with care to not wet the foliage for the first 24 hours after treatment. The effects on the plants 15 days after treatment were visually compared to untreated controls. Plant response ratings, listed in Table J as the means of the thee replicates, are based on a scale of 0 to 100 where 0 is no effect and 100 is complete control. Also listed in Table J are the expected effects for the mixtures calculated using Colby's Equation.

TABLE J
Observed and Expected Results from Compounds 2 and 6 Alone and in
Combination with Atrazine, Diuron, Hexazinone, Terbacil and Paraquat*

| Application Rate | | | DIGSA | | BRADC | | CHEAL | | AMARE | | SETFA | |
|---|---|---|---|---|---|---|---|---|---|---|---|---|
| (g a.i./ha) | | | Obsd. | Exp. | Obsd. | Exp. | Obsd. | Exp. | Obsd. | Exp. | Obsd. | Exp. |
| — | Atrazine | 125 | 5 | — | 15 | — | 67 | — | 38 | — | 0 | — |
| — | Atrazine | 250 | 5 | — | 5 | — | 95 | — | 75 | — | 18 | — |
| — | Diuron | 250 | 68 | — | 68 | — | 98 | — | 95 | — | 35 | — |
| — | Diuron | 500 | 63 | — | 85 | — | 100 | — | 100 | — | 67 | — |
| — | Hexazinone | 100 | 38 | — | 62 | — | 78 | — | 88 | — | 28 | — |
| — | Hexazinone | 200 | 53 | — | 70 | — | 87 | — | 80 | — | 45 | — |
| — | Terbacil | 250 | 45 | — | 75 | — | 93 | — | 63 | — | 18 | — |
| — | Terbacil | 500 | 48 | — | 73 | — | 100 | — | 97 | — | 55 | — |
| — | Paraquat | 31 | 78 | — | 67 | — | 95 | — | 90 | — | 72 | — |
| — | Paraquat | 62 | 85 | — | 95 | — | 100 | — | 100 | — | 88 | — |
| Cmpd 2 | | | | | | | | | | | | |
| 62 | — | | 5 | — | 8 | — | 40 | — | 47 | — | 28 | — |
| 125 | — | | 15 | — | 17 | — | 55 | — | 68 | — | 32 | — |
| 62 | Atrazine | 125 | 8 | 13 | 20 | 40 | 95 | 92 | 95 | 89 | 28 | 42 |
| 62 | Atrazine | 250 | 13 | 19 | 22 | 29 | 93 | 99 | 95 | 99 | 23 | 58 |
| 62 | Diuron | 250 | 45 | 24 | 85 | 93 | 98 | 100 | 98 | 100 | 63 | 70 |
| 62 | Diuron | 500 | 75 | 57 | 87 | 98 | 100 | 100 | 100 | 100 | 70 | 83 |
| 62 | Hexazinone | 100 | 48 | 17 | 73 | 98 | 87 | 98 | 87 | 95 | 37 | 55 |
| 62 | Hexazinone | 200 | 60 | 65 | 85 | 93 | 90 | 99 | 98 | 99 | 55 | 70 |
| 62 | Terbacil | 250 | 98 | 27 | 62 | 95 | 100 | 99 | 100 | 99 | 35 | 50 |
| 62 | Terbacil | 500 | 98 | 67 | 93 | 100 | 100 | 100 | 100 | 99 | 75 | 59 |
| 62 | Paraquat | 31 | 83 | 79 | 88 | 93 | 80 | 93 | 90 | 100 | 78 | 88 |
| 62 | Paraquat | 62 | 83 | 76 | 100 | 97 | 100 | 100 | 97 | 100 | 92 | 95 |
| Cmpd 6 | | | | | | | | | | | | |
| 62 | — | | 8 | — | 15 | — | 43 | — | 28 | — | 15 | — |
| 125 | — | | 13 | — | 40 | — | 68 | — | 60 | — | 33 | — |
| 62 | Atrazine | 125 | 8 | 13 | 35 | 28 | 87 | 81 | 80 | 55 | 20 | 15 |
| 62 | Atrazine | 250 | 15 | 13 | 23 | 19 | 98 | 97 | 98 | 82 | 42 | 30 |
| 62 | Diuron | 250 | 20 | 71 | 92 | 73 | 100 | 99 | 100 | 96 | 58 | 45 |
| 62 | Diuron | 500 | 55 | 66 | 98 | 87 | 100 | 100 | 100 | 100 | 77 | 72 |
| 62 | Hexazinone | 100 | 13 | 43 | 98 | 68 | 97 | 87 | 90 | 91 | 37 | 39 |
| 62 | Hexazinone | 200 | 63 | 57 | 92 | 75 | 98 | 93 | 98 | 86 | 58 | 53 |
| 62 | Terbacil | 250 | 23 | 49 | 95 | 79 | 98 | 96 | 98 | 73 | 30 | 30 |
| 62 | Terbacil | 500 | 65 | 52 | 100 | 77 | 100 | 100 | 98 | 98 | 43 | 62 |
| 62 | Paraquat | 31 | 78 | 80 | 92 | 72 | 88 | 97 | 100 | 93 | 83 | 76 |
| 62 | Paraquat | 62 | 75 | 86 | 97 | 96 | 100 | 100 | 100 | 100 | 93 | 90 |
| Application Rate | | | ABUTH | | XANST | | ECHCG | | ZEAMD | | IPOCO | |
| (g a.i./ha) | | | Obsd. | Exp. | Obsd. | Exp. | Obsd. | Exp. | Obsd. | Exp. | Obsd. | Exp. |
| — | Atrazine | 125 | 15 | — | 45 | — | 20 | — | 3 | — | 52 | — |
| — | Atrazine | 250 | 25 | — | 72 | — | 23 | — | 12 | — | 75 | — |
| — | Diuron | 250 | 77 | — | 93 | — | 62 | — | 20 | — | 92 | — |
| — | Diuron | 500 | 100 | — | 100 | — | 98 | — | 28 | — | 100 | — |
| — | Hexazinone | 100 | 82 | — | 100 | — | 15 | — | 45 | — | 92 | — |
| — | Hexazinone | 200 | 77 | — | 100 | — | 53 | — | 53 | — | 100 | — |
| — | Terbacil | 250 | 92 | — | 68 | — | 15 | — | 30 | — | 78 | — |
| — | Terbacil | 500 | 100 | — | 97 | — | 63 | — | 75 | — | 100 | — |
| — | Paraquat | 31 | 70 | — | 100 | — | 45 | — | 43 | — | 58 | — |
| — | Paraquat | 62 | 92 | — | 87 | — | 77 | — | 63 | — | 90 | — |
| Cmpd 2 | | | | | | | | | | | | |
| 62 | — | | 17 | — | 50 | — | 13 | — | 25 | — | 17 | — |
| 125 | — | | 18 | — | 58 | — | 8 | — | 65 | — | 27 | — |
| 62 | Atrazine | 125 | 48 | 42 | 88 | 92 | 28 | 37 | 23 | 42 | 60 | 78 |
| 62 | Atrazine | 250 | 62 | 69 | 90 | 96 | 57 | 78 | 20 | 39 | 83 | 82 |
| 62 | Diuron | 250 | 75 | 100 | 98 | 100 | 67 | 83 | 25 | 45 | 80 | 89 |
| 62 | Diuron | 500 | 100 | 92 | 98 | 100 | 90 | 94 | 55 | 55 | 100 | 98 |
| 62 | Hexazinone | 100 | 60 | 100 | 98 | 100 | 25 | 37 | 30 | 40 | 83 | 78 |
| 62 | Hexazinone | 200 | 88 | 98 | 100 | 100 | 45 | 52 | 30 | 46 | 98 | 96 |
| 62 | Terbacil | 250 | 100 | 100 | 98 | 100 | 90 | 67 | 25 | 51 | 90 | 82 |
| 62 | Terbacil | 500 | 100 | 100 | 98 | 100 | 100 | 97 | 60 | 61 | 95 | 89 |
| 62 | Paraquat | 31 | 43 | 82 | 88 | 100 | 48 | 89 | 47 | 42 | 50 | 89 |
| 62 | Paraquat | 62 | 88 | 98 | 100 | 100 | 77 | 89 | 42 | 42 | 100 | 98 |
| Cmpd 6 | | | | | | | | | | | | |
| 62 | — | | 27 | — | 67 | — | 17 | — | 22 | — | 22 | — |
| 125 | — | | 47 | — | 63 | — | 63 | — | 28 | — | 28 | — |
| 62 | Atrazine | 125 | 30 | 38 | 83 | 82 | 28 | 34 | 23 | 24 | 73 | 63 |
| 62 | Atrazine | 250 | 63 | 45 | 92 | 91 | 75 | 36 | 18 | 31 | 78 | 81 |
| 62 | Diuron | 250 | 100 | 83 | 100 | 98 | 80 | 68 | 27 | 38 | 87 | 94 |
| 62 | Diuron | 500 | 90 | 100 | 100 | 100 | 93 | 98 | 40 | 44 | 98 | 100 |
| 62 | Hexazinone | 100 | 100 | 87 | 100 | 100 | 28 | 29 | 20 | 57 | 73 | 94 |
| 62 | Hexazinone | 200 | 97 | 83 | 100 | 100 | 45 | 61 | 28 | 63 | 95 | 100 |
| 62 | Terbacil | 250 | 100 | 94 | 100 | 89 | 62 | 29 | 35 | 45 | 78 | 83 |
| 62 | Terbacil | 500 | 100 | 100 | 100 | 99 | 97 | 69 | 48 | 81 | 87 | 100 |
| 62 | Paraquat | 31 | 78 | 78 | 100 | 100 | 87 | 54 | 23 | 56 | 87 | 67 |
| 62 | Paraquat | 62 | 97 | 94 | 100 | 96 | 87 | 81 | 23 | 71 | 98 | 92 |

| | | | |
|---|---|---|---|
| Application Rate | AVEFA | TRZAS | ALOMY |

| (g a.i/ha) | | | Obsd. | Exp. | Obsd. | Exp. | Obsd. | Exp. |
|---|---|---|---|---|---|---|---|---|
| — | Atrazine | 125 | 13 | — | 3 | — | 3 | — |
| — | Atrazine | 250 | 28 | — | 8 | — | 28 | — |
| — | Diuron | 250 | 15 | — | 30 | — | 25 | — |
| — | Diuron | 500 | 47 | — | 40 | — | 43 | — |
| — | Hexazinone | 100 | 53 | — | 35 | — | 40 | — |
| — | Hexazinone | 200 | 62 | — | 60 | — | 78 | — |
| — | Terbacil | 250 | 68 | — | 55 | — | 62 | — |
| — | Terbacil | 500 | 97 | — | 90 | — | 97 | — |
| — | Paraquat | 31 | 42 | — | 35 | — | 60 | — |
| — | Paraquat | 62 | 63 | — | 83 | — | 73 | — |
| Cmpd 2 | | | | | | | | |
| 62 | — | | 8 | — | 8 | — | 15 | — |
| 125 | — | | 15 | — | 10 | — | 13 | — |
| 62 | Atrazine | 125 | 18 | 29 | 25 | 34 | 20 | 34 |
| 62 | Atrazine | 250 | 55 | 36 | 47 | 37 | 52 | 43 |
| 62 | Diuron | 250 | 30 | 51 | 33 | 61 | 32 | 58 |
| 62 | Diuron | 500 | 48 | 72 | 52 | 66 | 33 | 72 |
| 62 | Hexazinone | 100 | 52 | 74 | 47 | 68 | 58 | 70 |
| 62 | Hexazinone | 200 | 70 | 94 | 60 | 93 | 93 | 93 |
| 62 | Terbacil | 250 | 87 | 54 | 87 | 61 | 90 | 70 |
| 62 | Terbacil | 500 | 92 | 91 | 95 | 94 | 97 | 92 |
| 62 | Paraquat | 31 | 65 | 31 | 22 | 48 | 47 | 47 |
| 62 | Paraquat | 62 | 50 | 51 | 57 | 61 | 50 | 93 |
| Cmpd 6 | | | | | | | | |
| 62 | — | | 10 | — | 15 | — | 18 | — |
| 125 | — | | 25 | — | 30 | — | 33 | — |
| 62 | Atrazine | 125 | 23 | 22 | 28 | 18 | 22 | 20 |
| 62 | Atrazine | 250 | 30 | 35 | 32 | 22 | 33 | 41 |
| 62 | Diuron | 250 | 47 | 24 | 58 | 41 | 50 | 39 |
| 62 | Diuron | 500 | 70 | 52 | 63 | 49 | 67 | 53 |
| 62 | Hexazinone | 100 | 72 | 58 | 65 | 45 | 65 | 51 |
| 62 | Hexazinone | 200 | 93 | 66 | 92 | 66 | 92 | 82 |
| 62 | Terbacil | 250 | 50 | 71 | 58 | 62 | 65 | 69 |
| 62 | Terbacil | 500 | 90 | 97 | 93 | 92 | 90 | 98 |
| 62 | Paraquat | 31 | 25 | 48 | 43 | 45 | 38 | 67 |
| 62 | Paraquat | 62 | 47 | 67 | 58 | 86 | 92 | 78 |

*Application rates are grams of active ingredient per hectare (g a.i./ha).
“Obsd.” is observed effect.
“Exp.” is expected effect calculated from Colby's Equation.

As can be seen from the results listed in Table J, many of the effects observed were close to additive, but some combinations showed considerably greater than additive (i.e. synergistic) effects or less than additive (i.e. safening) on certain plant species. Particularly noteworthy greater than additive effects were observed for mixtures of Compound 2 with diuron and particularly terbacil on crabgrass, mixtures of Compound 6 with diuron, hexazinone and terbacil on Surinam grass, mixtures of Compound 6 with atrazine on pigweed, and mixtures of Compound 6 with terbacil on barnyardgrass. Some of the mixtures also showed a less than additive effect on wheat and particularly corn.

What is claimed is:

1. A compound selected from Formula I, an N-oxide or an agriculturally suitable salt thereof, 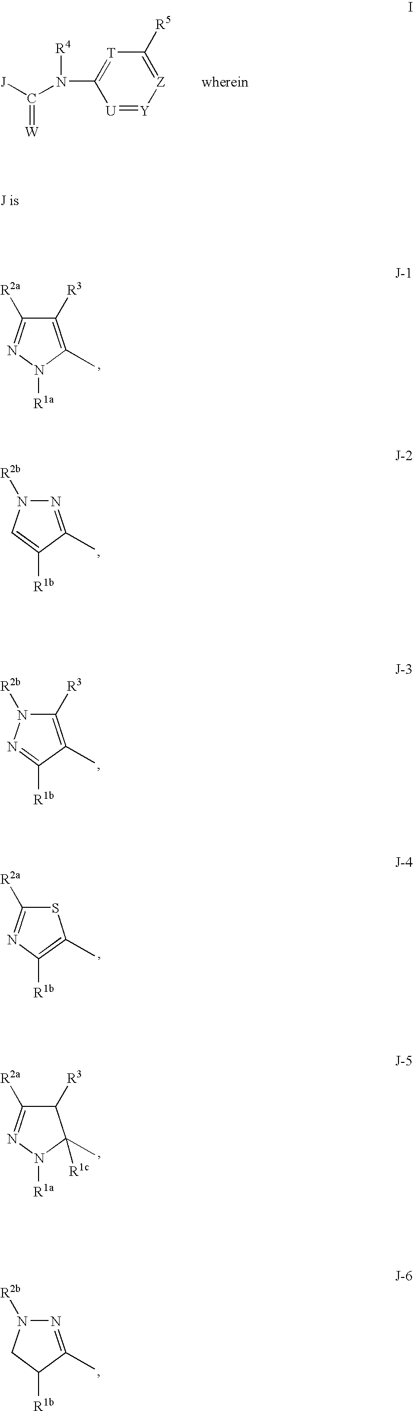 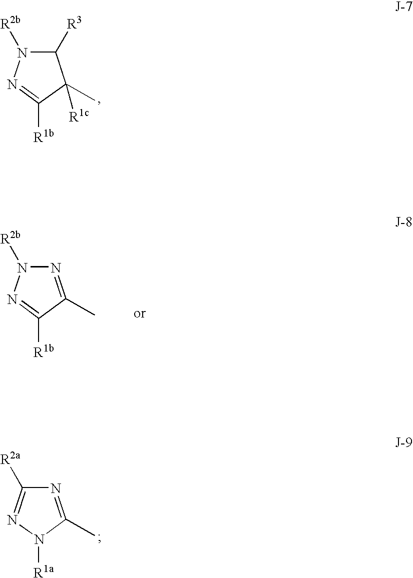
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂₋₄ alkenyl, C₂-C₄ fluoroalkenyl, C₂-C₄ alkynyl or C₂-C₄ fluoroalkynyl;
R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or
R^{2a} or R^{2b} is taken together with R³ as —C(R^{26a})(R^{26b})—(Y¹)ₛ—(CH₂)ₜ—(Y²)ᵤ— or —C(R^{26a})(R^{26b})—(Y¹)ₙ—CH═CH—(Y²)ₙ— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹ C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H, F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C₁-C₄ haloalkyl, C₂-C₄ alkenyl, C₃₋₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-c₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1;
provided that
(a) when J is J-1, R^{1a} is CH₃ and R⁵ is C(W¹)NR¹⁰R¹¹, C(O)OR¹², COR¹³, OR¹⁶ or S(O)ₘR¹⁷, then at least one of T, U, Y and Z is N or C—F;
(b) when J is J-1, R^{1a} is CH₃, R⁵ is C(W¹)NR¹⁰R¹¹, C(O)OR¹², COR¹³, OR¹⁶ or S(O)ₘR¹⁷ and T is N, then at least one of U, Y and Z is N or C—F;
(c) when J is J-1 and R^{2a} is CF₃, then R⁵ is C(W¹)NR¹⁰R¹¹ or C(NOR¹⁴)R¹⁵, or R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
(d) when J is J-3 and R^{1b} is CF₃, then R⁵ is C(W¹)NR¹⁰R¹¹ or C(NOR¹⁴)R¹⁵, or R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
(e) when J is J-1 or J-4 and R⁵ is OR¹⁶, then R^{2a} is other than CH₃;
(f) when J is J-3 and R⁵ is OR¹⁶ then R^{2b} is other than CH₃;
(g) when R⁵ is C(W¹)NR¹⁰R¹¹ or C(NOR¹⁴)R¹⁵, then R⁹ is other than alkoxy or alkylthio;
(h) when R⁵ is C(W)NR¹⁰R¹¹; then R⁶ is other than alkyl or alkoxy;
(i) when R⁵ is COR¹³, then R^{1a} or R^{1b} is selected from the radicals of the group consisting of C₁-C₃-alkyl, C₁-C₃ fluoroalkyl, C₂-C₃-alkenyl, C₂-C₃ fluoroalkenyl, C₂-C₃ alkynyl or C₂-C₃ fluoroalkynyl, each radical unbranched and connected through a terminal end carbon atom to the azole ring;
(j) when R⁵ and R⁶ are taken together as —C(W¹)N(R¹¹)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(k) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(l) when J is J-2 or J-6, then R⁷ and R⁹ are H;
(m) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(n) when R^{1a} is CH₃ and R⁵ is C(NOR¹⁴)R¹⁵, then R⁷ is other than alkyl;
(o) when T is N, then Z is CR⁹;
(p) when T is N and R⁷ is alkoxy, then R¹¹ is H;
(q) when R⁷ and R⁹ are F, and one of R¹⁰ and R¹¹ is H, then the other of R¹⁰ and R¹ is other than H;
(r) when Z is N and one of R¹⁰ and R¹¹ is H, then the other of R¹⁰ and R¹¹ is other than trifluoroethyl;
(s) when J is J-8 and R^{2b} is C₅-C₆ cycloalkyl, then R⁵ is C(O)NR¹⁰R¹¹;
(t) when J is J-8 and R⁷ is other than H, then R^{2b} is tert-butyl and R⁵ is C(O)NR¹⁰R¹¹; and
(u) when J is J-8, then R¹¹ is other than 1-ethylpropyl.

2. The compound of claim 1 wherein J is J-1, J-2, J-3, J-4, J-5, J-8 or J-9.

3. The compound of claim 2 wherein:
R^{1a} is CH₂CH₃, CH₂CH₂F, CH₂CHF₂, CH₂CF₃ or CH═CH₂;
R^{1b} is CH₃, CH₂CH₃, CH₂CH₂F, CH₂CHF₂, CH₂CF₃ or CH═CH₂;
R^{2a} or R^{2b} is tert-butyl, isopropyl or cyclopropyl;
R³ is H;
R⁴ is H; and
W is O.

4. The compound of claim 3 wherein at most one of T, U, Y and Z is N.

5. The compound of claim 4 wherein
R⁵ is CONR¹⁰R¹¹ or C(O)OR¹²;
R⁶ is H or F; and R⁷ is H or F;
R¹⁰ is H or C₁-C₄ alkyl;
R¹¹ is C₁-C₄ alkyl; or
R¹⁰ and R¹¹ are taken together as —CH₂CH═CHCH₂—; and
R¹² is C₁-C₃ alkyl.

6. The compound of claim 5 wherein J is J-1, J-3, J-5 or J-8.

7. The compound of claim 6 wherein
R⁵ is CONR¹⁰R¹¹;
R¹⁰ is H or C₁-C₂ alkyl; and
R¹¹ is C₁-C₃ alkyl; or
R¹⁰ and R¹¹ are taken together as —CH₂CH═CHCH₂—.

8. The compound of claim 7 wherein
R^{2a} is tert-butyl or isopropyl; and
R⁸ and R⁹ are H or F.

9. The compound of claim 8 which is selected from the group consisting of:
3-(1,1-dimethylethyl)-1-ethyl-N-[5-[(ethylamino)carbonyl)-2-fluorophenyl]-1H-pyrazole-5-carboxamide;
N-[5-[(dimethylamino)carbonyl]-2-fluorophenyl]-3-(1,1-dimethylethyl)-1-ethyl-1H-pyrazole-5-carboxamide;
2-[[(3-(1,1-dimethylethyl)-1-ethyl-1H-pyrazol-5-yl]carbonyl]amino]-N,N-dimethyl-4-pyridinecarboxamide;
2-[[[3-(1,1-dimethylethyl)-1-ethyl-1H-pyrazol-5-yl]carbonyl]amino]-N-ethyl-4-pyridinecarboxamide;
N-[5-[(dimethylamino)carbonyl]-2-fluorophenyl]-1-ethyl-3-(1-methylethyl)-1H-pyrazole-5-carboxamide;
N-[5-[(dimethylamino)carbonyl]-2-fluorophenyl]-1-(1,1-dimethylethyl)-3-ethyl-1H-pyrazole-4-carboxamide;
3-(1,1-dimethylethyl)-1-(2-fluoroethyl)-N-[3-[(1E)-1-(hydroxyimino)ethyl]phenyl]-1H-pyrazole-5-carboxamide;
3-(1,1-dimethylethyl)-1-ethyl-N-[5-[(ethylmethylamino)carbonyl]-2-fluorophenyl]-1H-pyrazole-5-carboxamide;
3-(1,1-dimethylethyl)-1-ethyl-N-[3-[(ethylamino)carbonyl]-4-fluorophenyl]-1H-pyrazole-5-carboxamide;
N-[5-[(2,5-dihydro-1H-pyrrol-0.1-yl)carbonyl]-2-fluorophenyl]-3-(1,1-dimethylethyl)-1-ethyl-1H-pyrazole-5-carboxamide;
3-(1,1-dimethylethyl)-1-ethyl-N-[3-(trifluoromethoxy)phenyl]-1H-pyrazole-5-carboxamide;
N-[3-[(dimethylamino)carbonyl]phenyl]-2-(1,1-dimethylethyl)-5-methyl-2H-1,2,3-triazole-4-carboxamide; and
N-[3-[(dimethylamino)carbonyl]phenyl]-2-(1,1-dimethylethyl)-5-ethyl-2H-1,2,3-triazole-4-carboxamide.

10. A herbicidal composition comprising a herbicidally effective amount of a compound of claim 1 and at least one of a surfactant, a solid diluent or a liquid diluent.

11. A method for controlling the growth of undesired vegetation comprising contacting the vegetation or its environment with a herbicidally effective amount of a compound of claim 1.

12. A method for controlling the growth of undesired vegetation comprising Urochloa decumbens (Staph) R. D. Webster comprising contacting the vegetation or its environment with herbicidally effective amounts of the compound of claim 1 which is N-[5-[(dimethylamino)carbonyl]-2-fluorophenyl]-3-(1,1-dimethylethyl)-1-ethyl-1H-pyrazole-5-carboxamide and at least one other herbicide selected from the group consisting of diuron and hexazinone.

13. A herbicidal mixture comprising a herbicidally effective amount of a compound of Formula Iz, an N-oxide or an agriculturally suitable salt thereof, 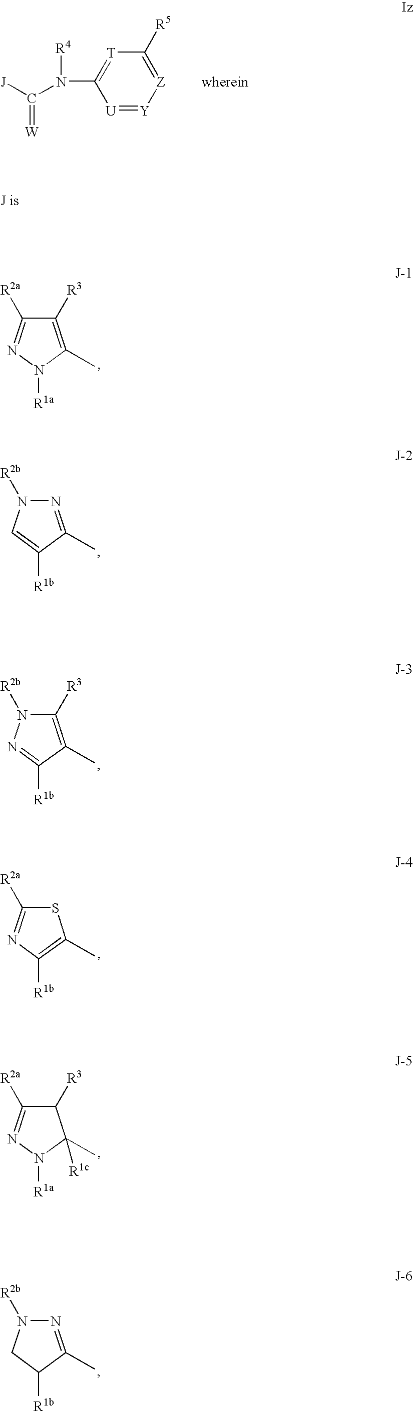 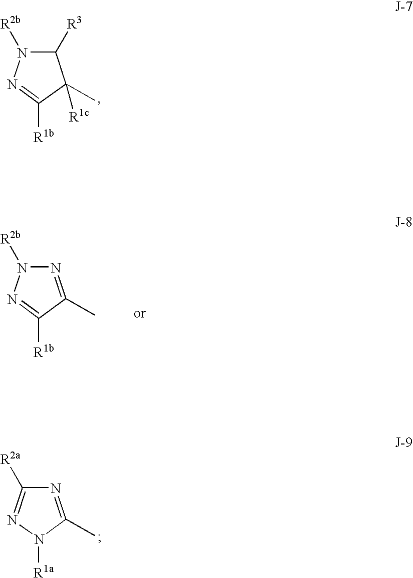
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or
R^{2a} or R^{2b} is taken together with R³ as —C(R^{26a})(R^{26b})—(Y¹)ₛ—(CH₂)ₜ—(Y₂)ᵤ— or —C(R^{26a})(R^{26b})—(Y¹)ᵥ—CH═CH—(Y²)_{w}— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹, C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H, F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C₁-C₄ haloalkyl, C₂-C₄ alkenyl, C₃-C₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1;
provided that
(a) when R⁵ is C(W¹)NR¹⁰R¹¹ or C(NOR¹⁴)R¹⁵, then R⁹ is other than alkoxy or alkylthio;
(b) when R⁵ is C(W¹)NR¹⁰R¹¹, then R⁶ is other than alkyl or alkoxy;
(c) when R⁵ is COR¹³, then R^{1a} or R^{1b} is selected from the radicals of the group consisting of C₁-C₃ alkyl, C₁-C₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring;
(d) when R⁵ and R⁶ are taken together as —C(W¹)N(R¹¹)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(e) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(f) when J is J-2 or J-6, then R⁷ and R⁹ are H;
(g) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(h) when R^{1a} is CH₃ and R⁵ is C(NOR¹⁴)R¹⁵, then R⁷ is other than alkyl;
(i) when T is N, then Z is CR⁹;
(j) when T is N and R⁷ is alkoxy, then R¹¹ is H;
(k) when R⁷ and R⁹ are F, and one of R¹⁰ and R¹¹ is H, then the other of R¹⁰ and R¹¹ is other than H;
(l) when Z is N and one of R¹⁰ and R¹¹ is H, then the other of R¹⁰ and R¹¹ is other than trifluoroethyl;
(m) when J is J-8 and R^{2b} is C₅-C₆ cycloalkyl, then R⁵ is C(O)NR¹⁰R¹¹;
(n) when J is J-8 and R⁷ is other than H, then R^{2b} is tert-butyl and R⁵ is C(O)NR¹⁰R¹¹; and
(o) when J is J-8, then R¹¹ is other than 1-ethylpropyl;
and an effective amount of at least one additional active ingredient selected from the group consisting of an other herbicide and a herbicide safener.

14. A herbicidal composition comprising the herbicidal mixture of claim 13 and at least one of a surfactant, a solid diluent or a liquid diluent.

15. A herbicidal mixture of claim 13 wherein the other herbicide is selected from atrazine, bromacil, diuron, hexazinone, terbacil, glyphosate, glufosinate, rimsulfuron, metsulfuron-methyl, sulfometuron-methyl, ametryn and paraquat.

16. A herbicidal mixture of claim 13 wherein the safener is selected from 1-bromo-4-[(chloromethyl)sulfonyl]benzene, cloquintocet-mexyl, cyometrinil, dichlormid, 2-(dichloromethyl)-2-methyl-1,3-dioxolane, fenchlorazole-ethyl, fenclorim, flurazole, fluxofenim, furilazole, isoxadifen-ethyl, mefenpyr-ethyl, (4-methoxy-3-methylphenyl)-(3-methylphenyl)methanone, 1,8-naphthalic anhydride and oxabetrinil.

17. A herbicidal mixture of claim 13 wherein the compound of Formula Iz is a compound of claim 1.

18. A method for selectively controlling the growth of undesired vegetation in a crop comprising contacting the locus of the crop with a herbicidally effective amount of a compound of Formula Iz, an N-oxide or an agriculturally suitable salt thereof, 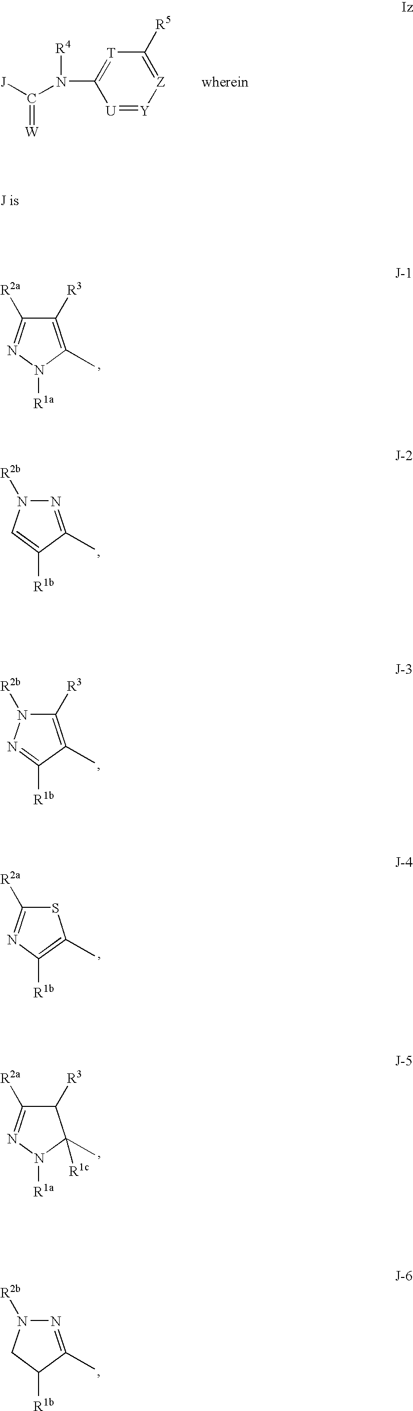 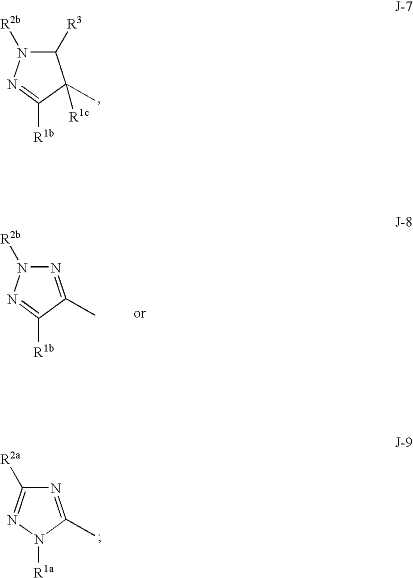
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or
R^{2a} or R^{2b} is taken together with R³ as —C(R^{26a})(R^{26b})—(Y¹)ₛ—(CH₂)ₜ—(Y²)ᵤ— or —C(R^{26a})(R^{26b})—(Y¹)ₙ—CH═CH—(Y²)_{w}— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹, C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H, F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C₁-C₄ haloalkyl, C₂-C₄ alkenyl, C₃-C₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1;
provided that
(a) when R⁵ is C(W¹)NR¹⁰R¹¹ or C(NOR¹⁴)R¹⁵, then R⁹ is other than alkoxy or alkylthio;
(b) when R⁵ is C(W¹)NR¹⁰R¹¹, then R⁶ is other than alkyl or alkoxy;
(c) when R⁵ is COR¹³, then R^{1a} or R^{1b} is selected from the radicals of the group consisting of C₁-C₃ alkyl, C₁-C₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring;
(d) when R⁵ and R⁶ are taken together as —C(W¹)N(R¹⁰)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(e) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(f) when J is J-2 or J-6, then R⁷ and R⁹ are H;
(g) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(h) when R^{1a} is CH₃ and R⁵ is C(NOR¹⁴)R¹⁵, then R⁷ is other than alkyl;
(i) when T is N, then Z is CR⁹;
(o) when T is N and R⁷ is alkoxy, then R¹¹ is H;
(k) when R⁷ and R⁹ are F, and one of R¹⁰ and R¹¹ is H, then the other of R¹⁰ and R¹ I is other than H;
(l) when Z is N and one of R¹⁰ and R¹¹ is H, then the other of R¹⁰ and R¹¹ is other than trifluoroethyl;
(m) when J is J-8 and R^{2b} is C₅-C₆ cycloalkyl, then R⁵ is C(O)NR¹⁰R¹¹;
(n) when J is J-8 and R⁷ is other than H, then R^{2b} is tert-butyl and R⁵ is C(O)NR¹⁰R¹¹; and
(O) when J is J-8, then R¹¹ is other than 1-ethylpropyl;
and an antidotally effective amount of a safener.

19. A method for selectively controlling the growth of undesired vegetation in a crop comprising contacting the locus of the crop with a herbicidally effective amount of a compound of Formula Iz, an N-oxide or an agriculturally suitable salt thereof, 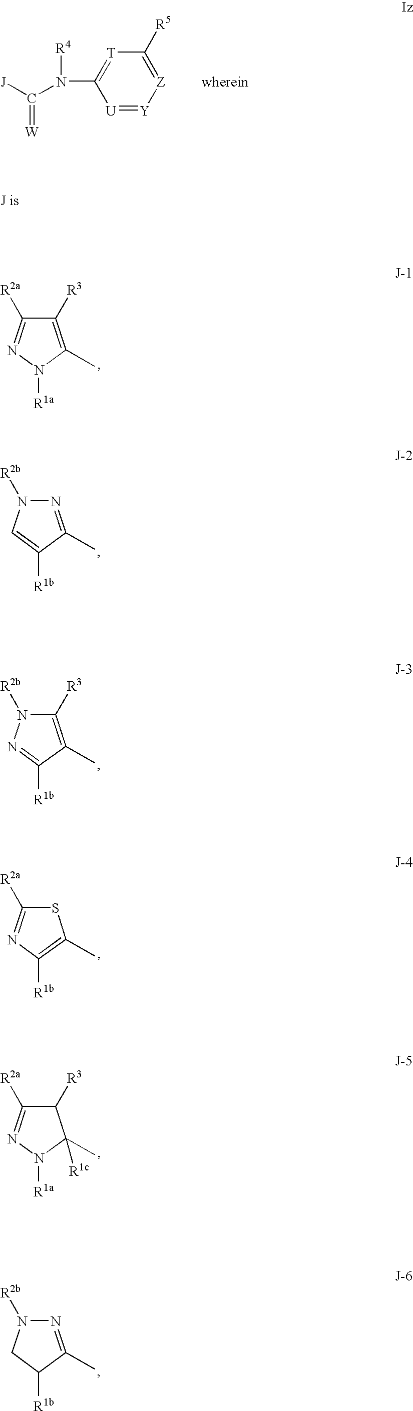 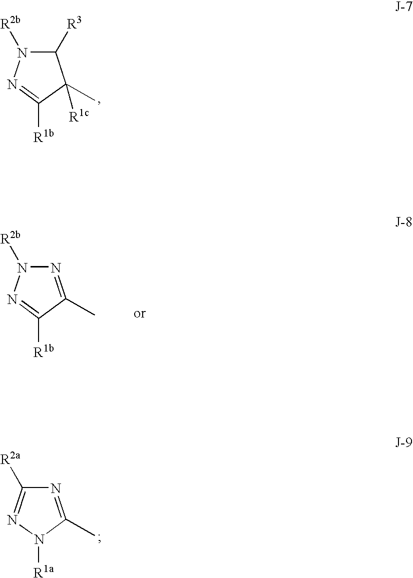
T is CR⁶ or N;
U is CR⁷ or N;
Y is CR⁸ or N;
Z is CR⁹ or N;
R^{1a} is H, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1b} is halogen, C₁-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
R^{1c} is H;
R^{2a} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl, C₅-C₆ alkylcycloalkylalkyl, —CR²⁰(OR²¹)(OR²²) or SiR²³R²⁴R²⁵;
R^{2b} is C₁-C₆ alkyl, C₁-C₆ haloalkyl, C₂-C₆ alkoxyalkyl, C₂-C₆ alkylthioalkyl, C₂-C₆ alkenyl, C₂-C₆ haloalkenyl, C₂-C₆ alkynyl, C₂-C₆ haloalkynyl, C₃-C₆ cycloalkyl, C₄-C₆ alkylcycloalkyl, C₃-C₆ halocycloalkyl, C₄-C₆ cycloalkylalkyl or C₅-C₆ alkylcycloalkylalkyl;
R³ is H, F or C₁-C₂ alkyl; or
R^{2a} or R^{2b} is taken together with R³ as —C(R^{26a})(R^{26b})—(Y¹)ₛ—(CH₂)ₜ—(Y²)ᵤ— or —C(R^{26a})(R^{26b})—(Y¹)ₙ—CH═CH—(Y²)_{w}— wherein the left end of the radical is connected as R^{2a} or R^{2b}, and the right end of the radical is connected as R³;
R⁴ is H, C₁-C₂ alkyl, C₂-C₆ alkylcarbonyl, C₂-C₆ alkoxycarbonyl, C₂-C₆ alkoxyalkyl or C₂-C₆ alkylthioalkyl;
R⁵ is C(W¹)NR¹⁰R¹¹, C(O)OR¹², COR¹³, C(NOR¹⁴)R¹⁵, —CN, OR¹⁶, S(O)ₘR¹⁷ S(O)₂NR¹⁸R¹⁹, OS(O)₂R²⁷ or OP(O)R^{28a}R^{28b};
R⁶ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio; or
R⁵ and R⁶ are taken together as a radical selected from —C(W¹)N(R¹¹)(CH₂)ₙ— and —C(NOR¹⁴)CH₂(CH₂)ₙ— wherein the right end of the radical is connected to the ring at T;
R⁷ is H, F, C₁-C₂ alkyl, C₁-C₂ fluoroalkyl, C₁-C₂ alkoxy, C₁-C₂ fluoroalkoxy, C₁-C₂ alkylthio or C₁-C₂ fluoroalkylthio;
R⁸ and R⁹ are independently selected from H. F, C₁-C₂ alkyl, C₁-C₂ fluoroalkyl, C₁-C₂ alkoxy, C₁-C₂ fluoroalkoxy, C₁-C₂ alkylthio and C₁-C₂ fluoroalkylthio;
R¹⁰ is H, C₁-C₄ alkyl, C₁-C₄ haloalkyl, C₃-C₄ alkenyl, C₂-C₄ alkoxymethyl or C₂-C₄ alkylthiomethyl;
R¹¹ is H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl, C₂-C₅ alkylthioalkyl, C₂-C₆ alkylcarbonyl or C₂-C₆ alkoxycarbonyl; or
R¹⁰ and R¹¹ are taken together as —(CH₂)₄—, —(CH₂)₅—, —CH₂CH═CHCH₂— or —(CH₂)₂O(CH₂)₂—, each optionally substituted with 1-2 C₁-C₂ alkyl;
each R¹² is independently C₁-C₅ alkyl, C₁-C₅ haloalkyl, C₂-C₄ alkoxyalkyl, C₂-C₄ alkylthioalkyl, C₂-C₅ alkenyl, C₃-C₅ haloalkenyl, C₃-C₅ alkynyl, C₃-C₅ cycloalkyl or C₄-C₅ cycloalkylalkyl;
R¹³ is C₁-C₃ alkyl, C₁-C₃ haloalkyl or cyclopropyl;
R¹⁴ is H, C₁-C₄ alkyl, C₁-C₄ haloalkyl, C₃-C₄ alkenyl, C₂-C₆ alkylcarbonyl or C₂-C₆ alkoxycarbonyl;
R¹⁵ is C₁-C₃ alkyl, C₁-C₃ haloalkyl or cyclopropyl;
R¹⁶ is C₁-C₄ alkyl, C₁-C₄ haloalkyl, C₂-C₃ alkoxyalkyl, C₂-C₃ alkylthioalkyl, C₂-C₄ alkenyl, C₃-C₄ haloalkenyl, C₃-C₄ alkynyl, C₃-C₅ cycloalkyl or cyclopropylmethyl;
R¹⁷ is C₁-C₄ alkyl, C—C₄ haloalkyl, C₂-C₄ alkenyl, C₃-C₄ haloalkenyl, C₃-C₄ alkynyl, C₃-C₄ cycloalkyl or cyclopropylmethyl;
each R¹⁸ is independently H, C₁-C₄ alkyl, C₁-C₄ haloalkyl, C₃-C₄ alkenyl, C₂-C₄ alkoxymethyl or C₂-C₄ alkylthiomethyl;
each R¹⁹ is independently H, C₁-C₅ alkyl, C₁-C₅ haloalkyl, C₂-C₅ alkenyl, C₃-C₅ haloalkenyl, C₃-C₅ alkynyl, C₃-C₅ cycloalkyl, C₄-C₅ cycloalkylalkyl, C₁-C₃ alkoxy, C₂-C₅ alkoxyalkyl or C₂-C₅ alkylthioalkyl;
R²⁰ is C₁-C₄ alkyl, cyclopropyl, cyclopropylmethyl or methylcyclopropyl;
R²¹ is C₁-C₃ alkyl;
R²² is C₁-C₃ alkyl; or
R²¹ and R²² are taken together as —CH₂CH₂— or —CH₂CH₂CH₂—, each optionally substituted with 1-2 methyl;
R²³ is C₁-C₂ alkyl or C₁-C₂ haloalkyl;
R²⁴ is C₁-C₂ alkyl or C₁-C₂ haloalkyl;
R²⁵ is C₁-C₂ alkyl or C₁-C₂ haloalkyl;
R^{26a} and R^{26b} are independently H or C₁-C₂ alkyl;
R²⁷ is C₁-C₃ alkyl, C₁-C₃ haloalkyl or cyclopropyl;
R^{28a} and R^{28b} are independently C₁-C₂ alkyl or C₁-C₂ alkoxy;
W is O or S;.
W¹ is O or S;
Y¹ and Y² are independently CH₂, O, S, NH or NCH₃;
m is 0, 1 or 2;
n is 1 or 2;
s is 0 or 1; t is 1 or 2; and u is 0 or 1; provided that the sum of s, t and u is 2 or 3; and
v is 0 or 1; w is 0 or 1; provided that the sum of v and w is 0 or 1;
provided that
(a) when R⁵ is C(W¹)NR¹⁰R¹¹ or C(NOR¹⁴)R¹⁵, then R⁹ is other than alkoxy or alkylthio;
(b) when R⁵ is C(W¹)NR¹⁰R¹¹, then R⁶ is other than alkyl or alkoxy;
(c) when R⁵ is COR¹³, then R^{1a} or R^{1b} is selected from the radicals of the group consisting of C₁-C₃ alkyl, C₁-C₃ fluoroalkyl, C₂-C₃ alkenyl, C₂-C₃ fluoroalkenyl, C₂-C₃ alkynyl or C₂-C₃ fluoroalkynyl, each radical unbranched and connected through a terminal end carbon atom to the azole ring;
(d) when R⁵ and R⁶ are taken together as —C(W¹)N(R¹⁰)(CH₂)ₙ— and n is 1, then R¹⁰ is C₁-C₄ alkyl, C₁-C₄ haloalkyl, C₃-C₄ alkenyl, C₂-C₄ alkoxymethyl or C₂-C₄ alkylthiomethyl;
(e) when at least one of R¹⁰ and R¹¹ is haloalkyl, then R^{1a} or R^{1b} is CH₂CH₃ or CH₂CF₃ and R^{2a} or R^{2b} is tert-butyl, isopropyl or cyclopropyl;
(f) when J is J-2 or J-6, then R⁷ and R⁹ are H;
(g) when J is J-2 or J-6, and R^{2b} is C₁-C₂ alkyl, then R^{1b} is halogen, C₂-C₄ alkyl, C₁-C₄ fluoroalkyl, C₂-C₄ alkenyl, C₂-C₄ fluoroalkenyl, C₂-C₄ alkynyl or C₂-C₄ fluoroalkynyl;
(h) when R^{1a} is CH₃ and R⁵ is C(NOR¹⁴)R¹⁵, then R⁷ is other than alkyl;
(i) when T is N, then Z is CR⁹;
(j) when T is N and R⁷ is alkoxy, then R¹¹ is H;
(k) when R⁷ and R⁹ are F, and one of R¹⁰ and R¹¹ is H, then the other of R¹⁰ and R¹¹ is other than H;
(l) when Z is N and one of R¹⁰ and R¹¹ is H, then the other of R¹⁰ and R¹¹ is other than trifluoroethyl;
(m) when J is J-8 and R^{2b} is C₅-C₆ cycloalkyl, then R⁵ is C(O)NR¹⁰R¹¹;
(n) when J is J-8 and R⁷ is other than H, then R^{2b} is tert-butyl and R⁵ is C(O)NR¹⁰R¹¹; and
(o) when J is J-8, then R¹¹ is other than 1-ethylpropyl;
wherein seed from which the crop is grown is treated with an antidotally effective amount of a safener.

20. The method of claim 19 wherein the safener comprises 1,8-naphthalic anhydride.

21. The method of claim 19 wherein the compound of Formula Iz is a compound of claim 1.
